(19) United States
(12) Patent Application Publication
(10) Pub. No.: US 2024/0150362 A1
(43) Pub. Date: May 9, 2024

(54) Substituted Pyrrolotriazines

(71) Applicant: Molecure S.A., Warsaw (PL)

(72) Inventors: Jacek Chrzanowski, Lodz (PL); Anna Gzik, Wartkowice (PL); Joanna Brzezinska, Konstantynow Lodzki (PL); Robert Koralewski, Lodz (PL); Lukasz Joachimiak, Lodz (PL); Karolina Rozga, Warszawa (PL); Szymon Kapuscinski, Lodz (PL); Bartlomiej Borek, Lodz (PL); Mateusz Urban, Katowice (PL); Sylwia Ciastek-Iskrzycka, Lodz (PL); Julita Nowicka, Wola Grzymkowa (PL); Kamil Lisiecki, Warszawa (PL); Roman Blaszczyk, Lodz (PL); Adam Golebiowski, Madison, CT (US); Jacek Olczak, Lodz (PL)

(21) Appl. No.: 18/455,978

(22) Filed: Aug. 25, 2023

Related U.S. Application Data
(60) Provisional application No. 63/400,902, filed on Aug. 25, 2022.

(30) Foreign Application Priority Data
Aug. 25, 2022 (PL) P.442089

(51) Int. Cl. C07D 487/04; A61K 45/06; C07D 519/00

(52) U.S. Cl. CPC C07D 487/04; A61K 45/06; C07D 519/00

(57) ABSTRACT

The inventive compounds are small molecule therapeutics that are potent inhibitors of USP7 activity. The invention also provides pharmaceutical compositions comprising the compounds, and methods for using the compounds for treating or preventing a disease, disorder, or condition associated with USP7 activity.

### RELATED APPLICATIONS

This application claims the benefit of priority to Polish Patent application number P.442089, filed Aug. 25, 2022, and U.S. Provisional Patent application No. 63/400,902, filed Aug. 25, 2022.

### BACKGROUND OF THE INVENTION

### Field of the Invention

This disclosure relates to small molecule therapeutic inhibitors of ubiquitin specific protease 7 (USP7), and methods of use thereof.

### Description of Related Art

Ubiquitin system is a major post-translational regulator of protein levels and regulates essential cellular processes in a cell. Ubiquitination is a covalent modification of a given protein that directs it towards proteasomal degradation. Ubiquitination as well as deubiquitination is tightly controlled by E1, E2 or E3 ligases and deubiquitinases (DUBs) respectively. Ubiquitination is usually done by formation of isopeptide bond on a protein's lysine side-chain. This process is further regulated by polyubiquitination during which a ubiquitin molecule's C-terminal Gly is conjugated with one of the seven Lys residues on another ubiquitin (Lys6, Lys11, Lys27, Lys29, Lys33, Lys48, or Lys63) or with the N-terminus to form linear chains. It is called “the ubiquitin code”. The ubiquitination code is related to diverse aspects of cellular biology and therefore disruption of this balance contributes to many diseases.

The ubiquitination is a reversible process and the deubiquitination is catalyzed by enzymes called deubiquitinases (DUBs). There are approximately 100 DUBs known in human genome, any of which could be playing a key role in the ubiquitin proteasome system as well as other biological processes. The DUBs are subdivided into five families: UCHs, USPs, OTUs, Josephin, and JAM/MPN+. Except the last one (which is metalloprotease), all DUBs are cysteine proteases consisting catalytic triad with cysteine residue as nucleophile. It is prone to covalent modifications by electrophilic fragments in small molecules and this has interfered with screening methodology and lead to identification of many low quality DUBs inhibitors in the past. However, recent years brought increasing appreciation of this family of targets once more thorough characterization of small molecule inhibitors of DUBs was performed. Deubiquitinases generally prevent protein degradation by cleaving the ubiquitin molecule or editing the polyubiquitin chains. Inhibition of DUBs therefore increases the rate of degradation of DUB-targeted protein. Regulation of DUBs enzymes with their inhibitors allow to indirectly affect the levels of protein of interest. Moreover the protein degradation is getting increasing attention as it might be an alternative strategy to overcome protein mutations and resistance to direct inhibitors.

Among USPs, a ubiquitin specific protease 7—USP7 has been intensively studied in recent years and numerous inhibitors of USP7 have been identified. Widely known function of USP7 is genetically validated interaction with an E3 ligase MDM2, which ubiquitinates and thus directs for degradation the known oncosupressor p53. MDM2 is deubiquitinated by USP7 resulting in its stabilization. Inhibition of USP7 has proven to restore ubiquitinylation of MDM2 and subsequent proteasomal degradation. This results in accumulation of p53 and promotes cell cycle arrest and apoptosis. On the other hand USP7 can stabilize p53 itself. [WO2020068600A1] Highly potent, specific, reversible, orally bioavailable USP7 inhibitors demonstrate marked tumor growth inhibition in both p53-wild type and p53-mutant tumors, indicating that USP7 inhibition can suppress tumor growth in vivo through both p53 dependent and independent mechanisms [Leger, P. L. et al. J. Med. Chem. 2020, 63, 5398-5420].

The further studies implicate numerous other partners for USP7 that are linked to DNA-damage response, cancer, immunotherapy, diabetes and viral infections [Nicholson B., Suresh Kumar K. G. The multifaceted roles of USP7; new therapeutic opportunities. Cell Biochemistry and Biophysics 2011, 60: 61-68.]. PTEN is thought to be a tumor suppressor in the nucleus. Its location is regulated by mono-ubiquitination. USP7 catalyzes the deubiquitylation nuclear PTEN which results in nuclear exclusion, blocking apoptosis in prostate cancer cells. That suggest that inhibition of USP7 triggers accumulation PTEN in nucleus and apoptosis. Other studies have uncovered a link between USP7 and Polycomb mediated silencing of genes. In particular, USP7 was shown to regulate the function of Ring1B and BMI1, which are essential core components of Polycomb Repressive Complex 1 and 2 [Gagarina, V. et al. J. Mol. Biol. 2020, 432, 4, 897-912.]. PRC 1 and 2 complexes catalyze the mono-, di- and trimethylation of lysine 27 of histone H3 (H3K27me1, H3K27me2 and H3K27me3) and is bound to CpG islands. H3K27me3 is a hallmark of PcG-associated transcriptional silencing and is thought to result in gene repression. The histone methyl transferase activity of PRC2 is mediated by one of the two catalytic subunits, enhancer of zeste homologue 1 (EZH1) or EZH2. Interaction between USP7 and PRC complexes, including EZH2 in particular but also other PRC components has been reported by several groups [De Bie, P. et al. Biochemical and Biophysical Research Communications 2010, 400, 3, 389-395.; Lecona, E. et al. Molecular and Cellular Biology 2015, 35, 7, 1157-1168.; Gagarina, V. et al. J. Mol. Biol. 2020, 432, 4, 897-912.]. Another epigenetic regulator—LSD1—was also reported to interact with USP7. Overexpression of LSD1 has been proved in numerous cancers, and high level of LSD1 aggressiveness and poor prognosis in lung, prostate, colon and breast cancers. The study shows that another oncogene—CARM1-dependendent methylation of LSD1 promotes deubiquitylation of LSD1 by USP7 [Liu, J. et al. EMBO Rep. 2020, 21(2), e48597.]. Wnt/beta-catenin/axin pathway plays important roles in many important biological processes and aberrant Wnt/β-catenin signaling has been associated with many human diseases, such as degenerative diseases and cancer. It was previously postulated that USP7 activates Wnt signaling and therefore inhibition of USP7 triggers Wnt attenuation. It was shown that USP7 stabilizes beta-catenin and once inhibited, beta-catenin is degraded and the canonical wnt pathway is inhibited [Novellasdemunt, L et al. Cell Reports 2017, 21(3), 612-627.]. However, recent study published by NIBR in Nat. Comm. shows that USP7 actually inhibits Wnt-induced beta-catenin accumulation [Ji, B. et al. Nature Communications 2019, 10, 4184.]. Both, genetic inhibition and pharmacological intervention on USP7 enhanced Wnt/B-catenin signaling with downstream effects on osteoblasts and adipocyte differentiation.

Importantly, USP7 plays a key role in determining half-life of crucial proteins involved in the regulation of immune response, namely FOXP3 and PD-L1 resulting in preservation of immunosuppressive functions of Tregs and causing escape of cancer cells from cytotoxic immune cells respectively. Evidence that USP7 by de-ubiquitylating of FOXP3 increases T Treg numbers and mediates suppression of tumor-infiltrating T effector cells has been elegantly demonstrated [Van Loosdregt, J. et al. Immunity 2013, 22, 39 (2), 259-271.]. This presented USP7 as an attractive immunoregulatory target. Indeed, USP7 deletion resulted in improved clinical outcome for many solid tumors. The observation that the accumulation of FOXP3+ Treg cells at the tumor or in draining lymph nodes signals poor prognosis highlights the significance of oncogenic mechanism of USP7.

As a part of immunotherapy, checkpoint blockades (PD-1/PD-L1) have acquired clinical success, antibody treatment has several limitations and need for small molecule is evident. Therefore the fact that USP7 has been demonstrated to be responsible for PD-L1 protein stabilization has serious therapeutic implications [Wang, Z. et al. Acta Pharm. Sin. B. 2021, 11, 3, 694-707.].

Overall it has been demonstrated that USP7 plays a critical role in affecting the tumour microenvironment. The effects of USP7 inhibition demonstrated promoting remodelling of the extracellular matrix (ECM), thereby promoting tumor invasion and metastasis. USP7 also affects angiogenesis and VEGF levels both systemically and in the tumor microenvironment (TME). Moreover, USP7 inhibition, particularly in the fibroblast compartment of the TME, leads to a significant decrease in both cell invasion and angiogenesis. USP7 inhibition also results in modulation of the tumor immune environment (e.g., by promoting infiltration of CD8 T cells). In vivo USP7 inhibition inhibits tumor growth in cell models that are not affected by direct inhibition by USP7 inhibitors in vitro. [WO2021161047A1]

Overall USP7 was found to be involved in many pathways that are aberrant in cancer and immune-oncology. Thus, inhibitors of USP7 can exert in vivo antitumor activity by: 1) directly inhibiting tumor cell proliferation via Hdm2 and other targets; 2) suppressing T regulatory cells via FOXP3, thereby facilitating the antitumor function of T effector cells; 3) inhibition of PD-L1 expression which sensitizes tumor cells towards cytotoxic effect of immune cells. First generation of USP7 inhibitors was often covalent (i.e., P5091 or HBX19818) or not selective. Next generation of allosteric and non-covalent inhibitors of USP7 was reported ([U.S. Ser. No. 11/084,829B2], [WO2023139241A1]) which exhibited in vivo efficacy.

Over the last years a potential of modulating immune system for the treatment of cancer has been described. A clinical success of immune checkpoint inhibitors, particularly those targeting the PD-1 axis, has set new trends in cancer therapy. Unfortunately, there is still unmet need and many patients do not improve after checkpoint blockade or other existing immunotherapies. T cell activation is crucial for the efficacy of immune checkpoint inhibitor therapies. The instant inventors believe that the disclosed compounds affect T cell activation, IFN-gamma production in particular. IFN-gamma is known to have anti-cancer properties, such as antiproliferative effect and induction of necroptosis in apoptosis-resistant cancer cells, regression of the tumor vasculature, activation antigen-presenting cells, as well as enhancement Th1 differentiation and cytotoxic T lymphocyte function.

Recently, a series of small-molecule inhibitors of USP7 activity was disclosed in the following patent applications: [WO2022048498A1], [WO2022170198A1], [WO2023003973A1], [WO2023139241A1].

Enhanced USP7 inhibitors are required, including those with better inhibitory activity, selectivity over other DUBs, increased solubility, enhanced in vitro and in vivo stability, improved pharmacokinetic profile, better safety profile, or all of these properties.

### SUMMARY OF THE INVENTION

In one aspect, the invention provides compounds of structural Formula (I):

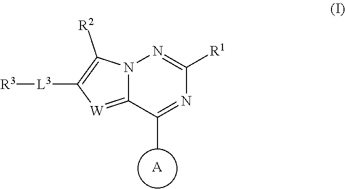

wherein:
   R¹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
   R² represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
   R³ represents N-containing heterocyclyl, (N-containing heterocyclyl)-NH—, or (N-containing heterocyclyl)-O—;
   or R³ represents —OH, —O—C₁-C₆ alkyl, —NH₂, —NH(—C₁-C₆ alkyl), or —N(—C₁-C₆ alkyl)₂;
   L³ represents a single bond, C₁-C₃ alkylene, or —C(═O)—;
   W represents C(—R⁴) or N;
   if present, R⁴ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
   and

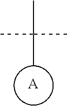

represents:
a)

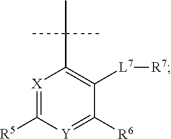

each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁶ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁷ represents N-containing heterocyclyl;
or R⁷ represents C₁-C₆ alkyl or H₂N—C₁-C₆ alkyl;
L⁷ represents a single bond, C₁-C₃ alkylene, —O—C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
b)

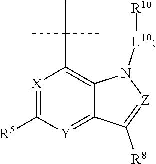

each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁸ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
Z represents C(—R⁹) or N;
if present, R⁹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R¹⁰ represents N-containing heterocyclyl;
or R¹⁰ represents —H or C₁-C₆ alkyl;
L¹⁰ represents a single bond, C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
c)

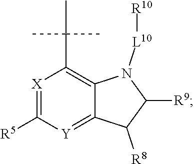

each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁸ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R¹⁰ represents N-containing heterocyclyl;
or R¹⁰ represents —H or C₁-C₆ alkyl;
L¹⁰ represents a single bond, C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
d)

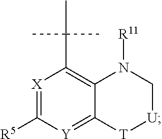

wherein each X and Y, independently, represents C(—R^{C});
each R^{C} represents —H;
R⁵ represents —Cl;
R¹¹ represents pyrrolidin-3-yl, piperidin-4-yl, morpholin-2-yl, or azetidin-3-yl; and
either T represents CH₂ or O, and U represents —CH₂— or CH(—CH₃),
or -T-U— represents

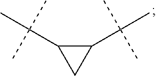

wherein each alkyl substituent independently represents a C₁-C₆ straight-chain alkyl or C₃-C₆ branched alkyl or C₃-C₆ cycloalkyl, as applicable, and is unsubstituted or substituted with one or more substituents, as valence permits, selected independently from the group consisting of deuterium, —F, —Cl, —Br, —I, —OH, —NH₂, —C(═O)—OH, heterocyclyl, and C₆-C₁₀ aryl;
wherein each N-containing heterocyclyl substituent independently represents a monocyclic or bicyclic non-aromatic ring system containing 4 to 9 ring member atoms including one or two nitrogen atoms and optionally one oxygen atom, and is optionally substituted with one or more substituents, as valence permits, selected independently from the group consisting of C₁-C₆ alkyl, —C≡N, —F, —Cl, —Br, —I, —CF₃, —OH, —NH₂, —C(═O)—OH, heterocyclyl, and C₆-C₁₀ aryl, and optionally one or two —CH₂— groups in the ring structure being replaced with —C(═O)— groups;
wherein each C₆-C₁₀ aryl substituent independently represents a monocyclic or bicyclic aromatic hydrocarbon ring system containing 6 to 10 ring member carbon atoms, and is unsubstituted or substituted with one or more substituents, as valence permits, selected independently from the group consisting of C₁-C₆ alkyl, —C≡N, —F, —Cl, —Br, —I, —OH, —NH₂, —C(═O)—OH, heterocyclyl, and C₆-C₁₀ aryl;
   and tautomers, stereoisomers, racemic or scalemic mixture of stereoisomers, pharmaceutically acceptable salts, esters, solvates, and polymorphs thereof.

Also provided herein are pharmaceutical compositions comprising (i) a therapeutically effective amount of at least one compound of the invention, or a pharmaceutically acceptable salt, solvate, or polymorph thereof; and (ii) a pharmaceutically acceptable carrier, vehicle or excipient therefor.

In another aspect, the invention provides methods for inhibiting USP7 in a cell or a tissue, comprising contacting the cell or the tissue with at least one compound according to the invention, or a pharmaceutically acceptable salt, solvate, or polymorph thereof, or with a pharmaceutical composition according to the invention.

In another aspect, the invention provides methods for the treatment or prevention of a disease, disorder, or condition associated with aberrant expression or activity of USP7, comprising administering to the subject in need thereof a therapeutically effective amount of at least one compound according to the invention, or a pharmaceutically acceptable salt, solvate, or polymorph thereof, or a pharmaceutical composition according to the invention.

In another aspect, the invention provides a compound according to the invention, or a pharmaceutically acceptable salt, solvate, or polymorph thereof, for use in a method for inhibiting USP7 in a cell or a tissue, comprising contacting the cell or the tissue with at least one compound according to the invention, or a pharmaceutically acceptable salt, solvate, or polymorph thereof.

In another aspect, the invention provides use of at least one compound according to the invention, or a pharmaceutically acceptable salt, solvate, or polymorph thereof, in the manufacturing of a medicament for the treatment of a disease, disorder, or condition associated with expression of USP7.

In further aspect, the invention provides a compound according to the invention, or a pharmaceutically acceptable salt, solvate, or polymorph thereof, for use in a method for the treatment or prevention of a disease, disorder, or condition associated with aberrant expression or activity of USP7, comprising administering to the subject in need thereof a therapeutically effective amount of at least one compound according to the invention, or a pharmaceutically acceptable salt, solvate, or polymorph thereof, or a pharmaceutical composition according to the invention.

In another aspect, the invention provides at least one compound according to the invention, or a pharmaceutically acceptable salt, solvate, or polymorph thereof, for use in the treatment or prevention of a disease, disorder, or condition selected from the group consisting of cardiovascular disorders, pulmonary disorders, autoimmune disorders, immune disorders, immunoregulatory disorders, neurodegenerative disorders, metabolic disorders, hemolytic disorders, gastrointestinal disorders, sexual disorders, infections, wound healing disorders, and cancers.

In another aspect, the invention provides methods for activating cytokine release in T lymphocyte cell, comprising contacting the cell with at least one compound according to the invention, or a pharmaceutically acceptable salt, solvate, or polymorph thereof, or with a pharmaceutical composition according to the invention.

In further aspect, the invention provides a compound according to the invention for use in a method for activating cytokine release in T lymphocyte cell in vitro or ex vivo, comprising contacting the cell with at least one compound according to the invention, or a pharmaceutically acceptable salt, solvate, or polymorph thereof.

In another aspect, the invention provides use of a compound according to the invention, or a pharmaceutically acceptable salt, solvate, or polymorph thereof, for protecting an organ during transport.

### DETAILED DESCRIPTION

The present invention is based on a surprising finding that some small molecule USP7 inhibitors possess very high activity accompanied by superior pharmacokinetics.

### Definitions

The articles “a” and “an” are used herein to refer to one or to more than one (i.e., to at least one) of the grammatical object of the article. By way of example, “an element” means one element or more than one element.

The terms used herein may be preceded and/or followed by a single dash “-”, or a double dash “=”, to indicate the bond order of the bond between the named substituent and its parent moiety; a single dash indicates a single bond and a double dash indicates a double bond. In the absence of a single or double dash, it is understood that a single bond is formed between the substituent and its parent moiety; further, substituents are intended to be read “from left to right,” unless a dash indicates otherwise. For example, C₁-C₆ alkoxycarbonyloxy and —OC(O)O—C₁-C₆ alkyl indicate the same functionality; similarly arylalkyl and -alkylaryl indicate the same functionality.

The terms “hydrogen”, “hydrogen atom”, and symbol “—H”, as used herein in the context of substituents to Markush formulas, such as Formula (I), denote a hydrogen atom attached to the remaining part of the molecule or group in question. For the sake of simplicity, hydrogen atoms attached to carbon atoms are not shown in the structural formulas; each carbon atom is understood to be associated with enough hydrogen atoms to give the carbon atom four bonds.

The term “alkyl” as used herein is a term of art and refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight-chain or branched-chain alkyl has about 30 or fewer carbon atoms in its backbone (e.g., C₁-C₃₀ for straight chain, C₃-C₃₀ for branched chain), and alternatively, about 20 or fewer, 10 or fewer. Preferred alkyl groups have 1-6 carbons. Representative examples of alkyl include, but are not limited to, methyl, ethyl, n-propyl, isopropyl, n-butyl, sec-butyl, isobutyl, tert-butyl, n-pentyl, isopentyl, neopentyl, and n-hexyl. Alkyl groups are independently optionally substituted with at least one substituent independently selected from the group consisting of oxo, fluoro, chloro, bromo, iodo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —CO₂H, —CONH₂, —NO₂, —SH, —SO₃H, —SO₄H, —SO₂NH₂, —NHNH₂, —ONH₂, —NHC(═O)—NHNH₂, —NHC(═O)—NH₂, —NHSO₂H, —NHC(═O)H, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ alkoxy, C₁-C₆ haloalkoxy, C₃-C₈ cycloalkyl, and C₆-C₁₀ aryl. Thus, —CF₃, —CH₂—CN, and —CH₂—OCF₃, are all examples of substituted C₁ alkyl.

The term “cycloalkyl” means monocyclic saturated or partially saturated carbocyclic rings, having from 3 to 8 carbon atoms, preferably from 3 to 6 carbon atoms, in their ring structure. Examples of monocyclic cycloalkyls include cyclopropyl, cyclobutyl, cyclopentyl, cyclopentenyl, cyclohexyl, and cyclohexenyl. Cycloalkyl groups are independently optionally substituted by at least one substituent independently selected from the group consisting of oxo, fluoro, chloro, bromo, iodo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —CO₂H, —CONH₂, —NO₂, —SH, —SO₃H, —SO₄H, —SO₂NH₂, —NHNH₂, —ONH₂, —NHC(═O)—NHNH₂, —NHC(═O)—NH₂, —NHSO₂H, —NHC(═O)H, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ alkyl, C₁-C₆ alkoxy, C₁-C₆ haloalkoxy, C₃-C₈ cycloalkyl, and C₆-C₁₀ aryl.

The term “heterocyclyl” as used herein refers to a radical of a non-aromatic ring system, including monocyclic rings, which can be completely saturated or which can contain one or more units of unsaturation, for the avoidance of doubt, the degree of unsaturation does not result in an aromatic ring system, and having 3 to 7 atoms including at least one heteroatom, such as nitrogen, oxygen, or sulfur. More preferred heterocycloalkyl groups have from 4-6 ring members where from 1-4 of the ring members are heteroatoms selected from the group comprising O, N, and S, the remaining ring atoms being C. For purposes of exemplification, which should not be construed as limiting the scope of this invention, the following are examples of heterocyclic rings: aziridinyl, azirinyl, oxiranyl, thiiranyl, thiirenyl, dioxiranyl, diazirinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, azetyl, oxetanyl, oxetyl, thietanyl, thietyl, diazetidinyl, dioxetanyl, dioxetenyl, dithietanyl, dithietyl, furyl, dioxalanyl, pyrrolyl, oxazolyl, thiazolyl, imidazolyl, oxadiazolyl, thiadiazolyl, triazolyl, triazinyl, isothiazolyl, isoxazolyl, thiophenyl, pyrazolyl, tetrazolyl, pyridyl, pyridazinyl, pyrimidinyl, pyrazinyl, triazinyl, tetrazinyl, azepinyl, azepanyl, azetidinyl, morpholinyl, oxadiazolinyl, oxadiazolidinyl, oxazolinyl, oxazolidinyl, oxopiperidinyl, oxopyrrolidinyl, piperazinyl, piperidinyl, pyranyl, pyrazolinyl, pyrazolidinyl, pyrrolinyl, pyrrolidinyl, thiomorpholinyl, tetrahydropyranyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. A heterocyclyl group is optionally substituted by one or more substituents independently selected from the group consisting of oxo, fluoro, chloro, bromo, iodo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —CO₂H, —CONH₂, —NO₂, —SH, —SO₃H, —SO₄H, —SO₂NH₂, —NHNH₂, —ONH₂, —NHC(═O)—NHNH₂, —NHC(═O)—NH₂, —NHSO₂H, —NHC(═O)H, —NHC(═O)—C₁-C₆ alkyl, —NH—C₁-C₆ alkyl, —N(—C₁-C₆ alkyl)₂, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ alkyl, C₁-C₆ alkoxy, C₁-C₆ haloalkoxy, C₃-C₈ cycloalkyl, and C₆-C₁₀ aryl.

A subset of heterocyclyl groups particularly preferred as substituents in the compounds of Formula (I) according to the invention has been identified and termed N-containing heterocyclyl substituents herein. Each N-containing heterocyclyl substituent independently represents a monocyclic or bicyclic non-aromatic ring system containing from 4 to 9 ring member atoms, preferably 4 or 5 or 6 ring member atoms, including one or two nitrogen atoms and optionally one oxygen atom, and is optionally substituted with one or more substituents, as valence permits, being selected independently from the group consisting of C₁-C₆ alkyl, —F, —Cl, —Br, —I, —CF₃, —OH, —NH₂, —C(═O)—OH, heterocyclyl, and C₆-C₁₀ aryl, and optionally one or two —CH₂— groups in the ring structure being replaced with —C(═O)— groups.

The term “heteroatom” is art-recognized, and includes an atom of any element other than carbon or hydrogen. Illustrative heteroatoms include oxygen, nitrogen and sulfur.

The term “cycloalkylalkyl” as used herein refers to an alkyl group substituted with one or more cycloalkyl groups.

The term “heterocycloalkylalkyl” as used herein refers to an alkyl group substituted with one or more heterocycloalkyl (i.e., heterocyclyl) groups.

The term “alkenyl” as used herein means a straight-chain or branched chain hydrocarbon radical containing from 2 to 6 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, and 5-hexenyl. The unsaturated bond(s) of the alkenyl group can be located anywhere in the moiety and can have either the (Z) or the (E) configuration about the double bond(s).

The term “alkylene” as used herein means a straight-chain or branched chain hydrocarbon diradical containing preferably from 1 to 3 carbon atoms and connected to the rest of molecule by two single bonds. Representative examples of C₁-C₃ alkylene include, but are not limited to, methylene (—CH₂—), ethane-1,2-diyl (—CH₂CH₂—), propane-1,1-diyl (>CHCH₂CH₃), and propane-1,3-diyl (—CH₂CH₂CH₂—).

The term “amino” is a term of art and as used herein refers to both unsubstituted and substituted amines, e.g., a moiety that may be represented by the general formulas:

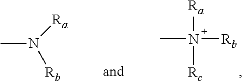

wherein Rₐ, R_{b}, and R_{c} each independently represent a hydrogen, an alkyl, an alkenyl, —(CH₂)ₓ—R_{d}, or Rₐ and R_{b}, taken together with the N atom to which they are attached complete a heterocycle having from 4 to 8 atoms in the ring structure; R_{d} represents an aryl, a cycloalkyl, a cycloalkenyl, or a heterocyclyl; and x is zero or an integer in the range of 1 to 8. In certain embodiments, only one of Rₐ or R_{b} may be a carbonyl, e.g., Rₐ, R_{b}, and the nitrogen together do not form an imide. In other embodiments, Rₐ and R_{b} (and optionally R_{c}) each independently represent a hydrogen, an alkyl, an alkenyl, or —(CH₂)ₓ—R_{d}. In certain embodiments, the term “amino” refers to —NH₂.

The term “carboxamido”, as used herein, means —NHC(═O)—, wherein the carboxamido group is bound to the parent molecular moiety through the nitrogen. Examples of carboxamido include alkylcarboxamido such as CH₃C(═O)—N(H)— and CH₃CH₂C(═O)—N(H)—.

The term “acyl” is a term of art and as used herein refers to any group or radical of the form RCO— where R is any organic group, e.g., alkyl, aryl, heteroaryl, aralkyl, and heteroaralkyl. Representative acyl groups include acetyl, benzoyl, and malonyl.

The term “aminoalkyl” as used herein refers to an alkyl group substituted with one or more one amino groups. In one embodiment, the term “aminoalkyl” refers to an aminomethyl group.

The term “azide” or “azido”, as used herein, means an —N₃ group.

The term “oxo” refers to the ═O radical.

The term “carbonyl” as used herein refers to —C(═O)—.

The term “alkylthio” as used herein refers to alkyl-S—.

The term “carboxy”, as used herein, means a —C(═O)—OH group.

The term “aryl” is a term of art and as used herein refers to includes monocyclic, bicyclic and polycyclic aromatic hydrocarbon groups, for example, benzene, naphthalene, anthracene, 1,2,3,4-tetrahydronaphthalene, indene, 2,3-dihydroindene, and pyrene. The aromatic ring may be substituted at one or more ring positions with one or more substituents, such as halogen, azide, alkyl, aralkyl, alkenyl, alkynyl, cycloalkyl, cycloalkylalkyl, (cycloalkyl)alkoxyl, hydroxyl, alkoxyl, amino, nitro, sulfhydryl, imino, carboxamido, phosphonate, phosphinate, carbonyl, carboxyl, silyl, ether, alkylthio, sulfonyl, aminosulfonyl, sulfonamido, ketone, aldehyde, ester, heterocyclyl, heterocyclylalkyl, aromatic or heteroaromatic moieties, aminoalkyl, haloalkyl, fluoroalkyl (such as trifluoromethyl), haloalkoxyl, cyano, or the like. The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, and/or heterocyclyls. Representative examples of the polycyclic aryl ring systems include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-6-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl, or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted. Preferred alkyl groups have 6-10 carbons in their aromatic ring structure, and are denoted as C₆-C₁₀ aryl groups. In certain embodiments, the term “aryl” refers to a phenyl group.

Aryl groups are optionally substituted by one or more substituents independently selected from the group consisting of fluoro, chloro, bromo, iodo, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —CO₂H, —CONH₂, —NO₂, —SH, —SO₃H, —SO₄H, —SO₂NH₂, —NHNH₂, —ONH₂, —NHC(═O)—NHNH₂, —NHC(═O)—NH₂, —NHSO₂H, —NHC(═O)H, —NHC(═O)—C₁-C₆ alkyl, —NH—C₁-C₆ alkyl, —N(—C₁-C₆ alkyl)₂, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ alkyl, C₁-C₆ alkoxy, C₁-C₆ haloalkoxy, C₃-C₈ cycloalkyl, and C₆-C₁₀ aryl.

The term “heteroaryl” is a term of art and as used herein refers to a monocyclic, bicyclic, and polycyclic aromatic group having 3 to 14, 5 to 14, or 3 to 12 total atoms including one or more heteroatoms such as nitrogen, oxygen, or sulfur in the ring structure. More preferred heteroaryl groups have from 5-10 ring members where from 1-4 of the ring members are heteroatoms selected from the group comprising O, N, and S. Exemplary heteroaryl groups include, for example, azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl, and the like. The “heteroaryl” may be substituted at one or more ring positions with one or more substituents such as halogen, azido, alkyl, aralkyl, alkenyl, alkynyl, cycloalkyl, hydroxyl, alkoxyl, amino, nitro, sulfhydryl, imino, carboxamido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, and/or heterocyclyls. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, and 6,7-dihydrobenzo[c][1,2,5]oxadiazol-4(5H)-onyl. Any bicyclic heteroaryl can be optionally substituted as detailed for “heteroaryl” above.

The term “aralkyl”, “arylalkyl”, or “aryl-C₁-C₆ alkyl” is a term of art and as used herein refers to an alkyl group, for example a C₁-C₆ alkyl group, substituted with an aryl group, wherein the moiety is appended to the parent molecule through the alkyl group.

The term “heteroaralkyl”, “heteroarylalkyl”, or “heteroaryl-C₁-C₆ alkyl” is a term of art and as used herein refers to an alkyl group, for example a C₁-C₆ alkyl group, substituted with a heteroaryl group, appended to the parent molecular moiety through the alkyl group.

The term “alkoxy” or “alkoxyl” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxycarbonyl” means an alkoxy group, as defined herein, appended to the parent molecular moiety through a carbonyl group, represented by —C(═O)—, as defined herein. Representative examples of alkoxycarbonyl include, but are not limited to, methoxycarbonyl, ethoxycarbonyl, and tert-butoxycarbonyl.

The term “alkylcarbonyl”, as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “arylcarbonyl”, as used herein, means an aryl group, as defined herein, appended to the parent molecular moiety through a carbonyl group, as defined herein. Representative examples of arylcarbonyl include, but are not limited to, benzoyl and (2-pyridinyl)carbonyl.

The term “alkylcarbonyloxy” and “arylcarbonyloxy”, as used herein, means an alkylcarbonyl or arylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. Representative examples of arylcarbonyloxy include, but are not limited to phenylcarbonyloxy.

The term “aryloxy” as used herein means an aryl group, as defined herein, appended to the parent molecular moiety through an oxygen atom.

The term “heteroaryloxy” as used herein means a heteroaryl group, as defined herein, appended to the parent molecular moiety through an oxygen atom.

The terms “cyano” and “nitrile” are terms of art and as used herein refer to —C≡N, denoted also as —CN.

The term “nitro”, as used herein, means —NO₂.

The terms “halo” and “halogen” are terms of art and as used herein refer to —F, —Cl, —Br, or —I.

The term “haloalkyl” as used herein refers to an alkyl group, as defined herein, wherein some or all of the hydrogens are replaced with halogen atoms. The term “haloalkoxyl” refers to an alkoxy group, as defined herein, wherein some or all of the hydrogens are replaced with halogen atoms. An exemplary haloalkyl group is trifluoromethyl.

The terms “hydroxy” and “hydroxyl” are a term of art and as used herein refer to —OH.

The term “hydroxyalkyl”, as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

Certain compounds contained in compositions of the present invention may exist in particular geometrical isomer or stereoisomeric forms. In addition, compounds of the present invention may also be optically active. The present invention contemplates all such compounds, including cis- and trans-isomers, (R)- and (S)-enantiomers, diastereoisomers, D-isomers, L-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, halogen, azide, alkyl, aralkyl, alkenyl, alkynyl, cycloalkyl, cycloalkylalkyl, (cycloalkyl)alkoxyl, hydroxyl, alkoxyl, amino, nitro, sulfhydryl, imino, carboxamido, phosphonate, phosphinate, carbonyl, carboxyl, silyl, ether, alkylthio, sulfonyl, aminosulfonyl, sulfonamido, ketone, aldehyde, ester, heterocyclyl, heterocyclylalkyl, aromatic or heteroaromatic moieties, aminoalkyl, haloalkyl, fluoroalkyl (such as trifluoromethyl), haloalkoxyl, cyano, or other substituents described above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group”, as used herein, means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

A “saturated” or “fully saturated” compound means that the referenced chemical structure does not contain any multiple carbon-carbon bonds. For example, a saturated cycloalkyl group as defined herein includes cyclohexyl, cyclopropyl, and the like.

An “unsaturated” or “partially saturated” compound means that the referenced chemical structure may contains on or more multiple carbon-carbon bonds, but is not aromatic. For example, an unsaturated cycloalkyl group as defined herein includes cyclohexenyl, cyclopentenyl, cyclohexadienyl, and the like.

For purposes of the invention, the chemical elements are identified in accordance with the Periodic Table of the Elements, CAS version, Handbook of Chemistry and Physics, 67th ed., 1986-87, inside cover.

It will be apparent to one skilled in the art that certain compounds of this disclosure may exist in tautomeric forms, all such tautomeric forms of the compounds being within the scope of the disclosure. A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of tautomers will exist.

The deuterated derivatives of the present compounds are also within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. Both the R and the S stereochemical isomers, as well as all mixtures thereof, are included within the scope of the disclosure. A “racemic mixture” or “racemate” denotes equimolar mixture of two enantiomers, whereas a “scalemic mixture” denotes non-racemic mixture of two enantiomers.

The chemical structure of examples that are a mixture of diastereoisomers or a single diastereoisomer but with unknown relative configuration are drawn and named without defined stereochemical configuration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, pamoic (embonic), succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula (I). As another example, the salt may comprise less than one inorganic or organic acid molecule per molecule of base, such as two molecules of compound of Formula (I) per molecule of tartaric acid.

The compounds according to the invention, e.g., the compounds of Formula (I), can form solvates with a stoichiometric or non-stoichiometric amount of one or more solvents, such as water, ethanol, diethyl ether, or ethyl acetate. The solvates formed with water ale called hydrates.

As used herein, a protic solvent is a solvent that has a hydrogen atom bound to an oxygen atom (as in a hydroxyl group) or a nitrogen atom (as in an amine group). In general terms, any solvent that contains labile H⁺ is called a protic solvent. The molecules of such solvents readily donate protons (H⁺) to reagents. In contrast, an aprotic solvent is a solvent that does not have a hydrogen atom bound to an oxygen (as in a hydroxyl group) or a nitrogen (as in an amine group), and it cannot donate hydrogen.

As used herein, a polar protic solvent is a protic solvent that will dissolve many salts. In general, these solvents have high dielectric constants and high polarity. Non-limiting examples of polar protic solvents include acetic acid, ammonia, ethanol, formic acid, isopropanol, methanol, n-butanol, nitromethane, n-propanol, tert-butanol, and water.

As used herein, a polar aprotic solvent is a solvent that will dissolve many salts, but lacks an acidic hydrogen; these solvents generally have intermediate to high dielectric constants and polarity. Non-limiting examples of polar aprotic solvents include acetone, acetonitrile, dichloromethane (DCM), dimethyl sulfoxide (DMSO), ethyl acetate, hexamethylphosphoric triamide (HMPT), N,N-dimethylformamide (DMF), and tetrahydrofuran (THF).

As used herein, a nonpolar aprotic solvent is a solvent that will dissolve many salts, but lacks an acidic hydrogen; these solvents generally have low dielectric constants and polarity. Non-limiting examples of nonpolar aprotic solvents include benzene, chloroform, cyclohexane, diethyl ether, hexane, pentane, and toluene.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, the mode of administration, the bioavailability of the particular compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

“Modulating” or “modulate” refers to the treating, prevention, suppression, enhancement or induction of a function, condition or disorder.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, in other words—preventive, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). Thus, in certain aspects, the disclosure encompasses non-prophylactic treatment, i.e., therapeutic treatment.

As used herein, “subject” refers to a warm blooded animal such as a mammal, preferably a human, or a human child, which is afflicted with, or has the potential to be afflicted with one or more diseases and disorders described herein.

“EC₅₀” refers to a dosage, concentration or amount of a particular test compound that elicits a dose-dependent response at 50% of maximal expression of a particular response that is induced, provoked or potentiated by the particular test compound.

“IC₅₀” refers to an amount, concentration or dosage of a particular test compound that achieves a 50% inhibition of a maximal response in an assay that measures such response.

### Compounds of the Invention

In one aspect, the invention provides a compound of structural Formula (I):

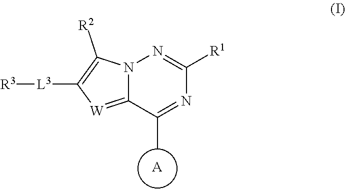

wherein:
   R¹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
   R² represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
   R³ represents N-containing heterocyclyl, (N-containing heterocyclyl)-NH—, or (N-containing heterocyclyl)-O—;
   or R³ represents —OH, —O—C₁-C₆ alkyl, —NH₂, —NH(—C₁-C₆ alkyl), or —N(—C₁-C₆ alkyl)₂;
   L³ represents a single bond, C₁-C₃ alkylene, or —C(═O)—;
   W represents C(—R⁴) or N;
   if present, R⁴ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
   and

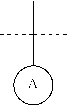

represents:
a)

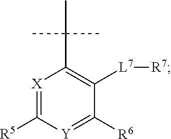

each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁶ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁷ represents N-containing heterocyclyl;
or R⁷ represents C₁-C₆ alkyl or H₂N—C₁-C₆ alkyl;
L⁷ represents a single bond, C₁-C₃ alkylene, —O—C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
b)

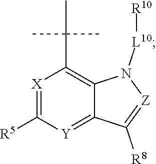

each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁸ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
Z represents C(—R⁹) or N;
if present, R⁹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R¹⁰ represents N-containing heterocyclyl;
or R¹⁰ represents —H or C₁-C₆ alkyl;
L¹⁰ represents a single bond, C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
c)

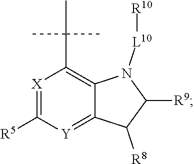

each X an Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁸ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R¹⁰ represents N-containing heterocyclyl;
or R¹⁰ represents —H or C₁-C₆ alkyl;
L¹⁰ represents a single bond, C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
d)

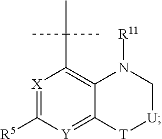

wherein each X and Y, independently, represents C(—R^{C});
each R^{C} represents —H;
R⁵ represents —Cl;
R¹¹ represents pyrrolidin-3-yl, piperidin-4-yl, morpholin-2-yl, or azetidin-3-yl; and
either T represents CH₂ or O, and U represents —CH₂— or CH(—CH₃),
or -T-U— represents

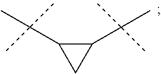

wherein each alkyl substituent independently represents a C₁-C₆ straight-chain alkyl or C₃-C₆ branched alkyl or C₃-C₆ cycloalkyl, as applicable, and is unsubstituted or substituted with one or more substituents, as valence permits, selected independently from the group consisting of deuterium, —F, —Cl, —Br, —I, —OH, —NH₂, —C(═O)—OH, heterocyclyl, and C₆-C₁₀ aryl;
wherein each N-containing heterocyclyl substituent independently represents a monocyclic or bicyclic non-aromatic ring system containing 4 to 9 ring member atoms including one or two nitrogen atoms and optionally one oxygen atom, and is optionally substituted with one or more substituents, as valence permits, selected independently from the group consisting of C₁-C₆ alkyl, —C≡N, —F, —Cl, —Br, —I, —CF₃, —OH, —NH₂, —C(═O)—OH, heterocyclyl, and C₆-C₁₀ aryl, and optionally one or two —CH₂— groups in the ring structure being replaced with —C(═O)— groups;
wherein each C₆-C₁₀ aryl substituent independently represents a monocyclic or bicyclic aromatic hydrocarbon ring system containing 6 to 10 ring member carbon atoms, and is unsubstituted or substituted with one or more substituents, as valence permits, selected independently from the group consisting of C₁-C₆ alkyl, —C≡N, —F, —Cl, —Br, —I, —OH, —NH₂, —C(═O)—OH, heterocyclyl, and C₆-C₁₀ aryl;
   or a tautomer, stereoisomer, a racemic or scalemic mixture of stereoisomers, a pharmaceutically acceptable salt, ester, solvate, or polymorph thereof.

In some preferred embodiments, the invention provides a compound of structural Formula (I):

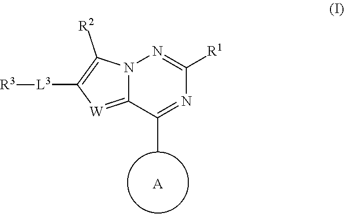

wherein:
   R¹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
   R² represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
   R³ represents N-containing heterocyclyl, (N-containing heterocyclyl)-NH—, or (N-containing heterocyclyl)-O—;
   or R³ represents —OH, —O—C₁-C₆ alkyl, —NH₂, —NH(—C₁-C₆ alkyl), or —N(—C₁-C₆ alkyl)₂; and
   L³ represents a single bond, C₁-C₃ alkylene, or —C(═O)—;
   W represents C(—R⁴) or N;
   if present, R⁴ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
   and

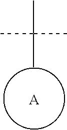

represents:
a)

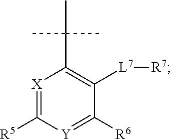

each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁶ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁷ represents N-containing heterocyclyl;
or R⁷ represents C₁-C₆ alkyl;
L⁷ represents a single bond, C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
b)

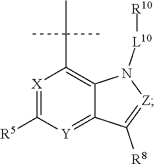

each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁸ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
Z represents C(—R⁹) or N;
if present, R⁹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R¹⁰ represents N-containing heterocyclyl;
or R¹⁰ represents —H or C₁-C₆ alkyl;
L¹⁰ represents a single bond, C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
c)

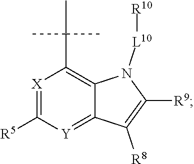

each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁸ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R¹⁰ represents N-containing heterocyclyl;
or R¹⁰ represents —H or C₁-C₆ alkyl;
L¹⁰ represents a single bond, C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
wherein each alkyl substituent independently represents a C₁-C₆ straight-chain alkyl or C₃-C₆ branched alkyl or C₃-C₆ cycloalkyl, as applicable, and is unsubstituted or substituted with one or more substituents, as valence permits, selected independently from the group consisting of —F, —Cl, —Br, —I, —OH, —NH₂, —C(═O)—OH, heterocyclyl, and C₆-C₁₀ aryl;
wherein each N-containing heterocyclyl substituent independently represents a monocyclic or bicyclic non-aromatic ring system containing 4 or 5 or 6 ring member atoms including one or two nitrogen atoms and optionally one oxygen atom, and is optionally substituted with one or more substituents, as valence permits, selected independently from the group consisting of C₁-C₆ alkyl, —C≡N, —F, —Cl, —Br, —I, —CF₃, —OH, —NH₂, —C(═O)—OH, heterocyclyl, and C₆-C₁₀ aryl, and optionally one or two —CH₂— groups in the ring structure being replaced with —C(═O)— groups;
wherein each C₆-C₁₀ aryl substituent independently represents a monocyclic or bicyclic aromatic hydrocarbon ring system containing 6 to 10 ring member carbon atoms, and is unsubstituted or substituted with one or more substituents, as valence permits, selected independently from the group consisting of C₁-C₆ alkyl, —C≡N, —F, —Cl, —Br, —I, —OH, —NH₂, —C(═O)—OH, heterocyclyl, and C₆-C₁₀ aryl;
   or a tautomer, stereoisomer, a racemic or scalemic mixture of stereoisomers, a pharmaceutically acceptable salt, ester, solvate, or polymorph thereof.

In some preferred embodiments, the invention provides a compound of Formula (I), as defined above, or a tautomer, stereoisomer, a racemic or scalemic mixture of stereoisomers, a pharmaceutically acceptable salt, ester, solvate, or polymorph thereof, wherein:

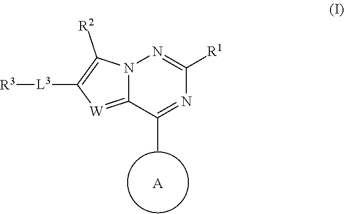

wherein:
   R¹ represents —H or C₁-C₆ alkyl;
   R² and R⁴, each independently, represent —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
   R³ represents N-containing heterocyclyl;
   or R³ represents —OH, —O—C₁-C₆ alkyl, —NH₂, —NH(—C₁-C₆ alkyl), or —N(—C₁-C₆ alkyl)₂;
   W represents C(—R⁴);
   R⁴ represents —H or C₁-C₆ alkyl;
   and

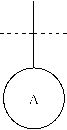

represents:
a)

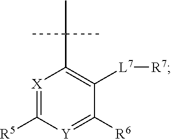

each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents C₁-C₆ alkyl, —C≡N, —N₃, —NO₂, —F, —Cl, —Br, or —CF₃;
R⁶ represents C₁-C₆ alkyl, —C≡N, —N₃, —NO₂, —F, —Cl, —Br, or —CF₃;
R⁷ represents N-containing heterocyclyl;
or R⁷ represents C₁-C₆ alkyl; and
L⁷ represents a single bond, C₁-C₃ alkylene, —O—, —S—, —S(═O)—, —S(═O)₂—, —C(═O)—, or —CH(—OH)—;
or:
b)

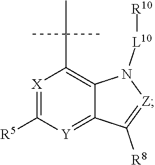

each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents C₁-C₆ alkyl, —C≡N, —N₃, —NO₂, —F, —Cl, —Br, —I, or —CF₃;
R⁸ represents —H, C₁-C₆ alkyl, —C≡N, —N₃, —NO₂, —F, —Cl, —Br, —I, or —CF₃;
Z represents C(—R⁹) or N;
if present, R⁹ represents —H, C₁-C₆ alkyl, —C≡N, —N₃, —NO₂, —F, —Cl, —Br, —I, or —CF₃;
R¹⁰ represents N-containing heterocyclyl;
or R¹⁰ represents —H or C₁-C₆ alkyl; and
L¹⁰ represents a single bond, C₁-C₃ alkylene, —O—, —S—, —S(═O)—, —S(═O)₂—, —C(═O)—, or —CH(—OH)—;
or:
c)

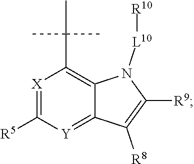

each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents C₁-C₆ alkyl, —C≡N, —N₃, —NO₂, —F, —Cl, —Br, —I, or —CF₃;
R⁸ represents —H, C₁-C₆ alkyl, —C≡N, —N₃, —NO₂, —F, —Cl, —Br, —I, or —CF₃;
R⁹ represents —H, C₁-C₆ alkyl, —C≡N, —N₃, —NO₂, —F, —Cl, —Br, —I, or —CF₃;
R¹⁰ represents N-containing heterocyclyl;
or R¹⁰ represents —H or C₁-C₆ alkyl; and
L¹⁰ represents a single bond, C₁-C₃ alkylene, —O—, —S—, —S(═O)—, —S(═O)₂—, —C(═O)—, or —CH(—OH)—;
wherein each alkyl substituent independently represents C₁-C₆ straight-chain alkyl or C₃-C₆ branched alkyl or C₃-C₆ cycloalkyl, as applicable, and is unsubstituted or substituted with one or more substituents, as valence permits, selected independently from the group consisting of —F, —Cl, —Br, —I, —OH, —NH₂, —C(═O)—OH, heterocyclyl, and C₆-C₁₀ aryl;
wherein each N-containing heterocyclyl substituent independently represents a monocyclic or bicyclic non-aromatic ring system containing 4 or 5 or 6 ring member atoms including one or two nitrogen atoms and optionally one oxygen atom, and is optionally substituted with one or more substituents, as valence permits, selected independently from the group consisting of C₁-C₆ alkyl, —C≡N, —F, —Cl, —Br, —I, —CF₃, —OH, —NH₂, —C(═O)—OH, heterocyclyl, and C₆-C₁₀ aryl, and optionally one or two —CH₂— groups in the ring structure being replaced with —C(═O)— groups.

In some preferred embodiments, the invention provides a compound of Formula (I), as defined above, or a tautomer, stereoisomer, a racemic or scalemic mixture of stereoisomers, a pharmaceutically acceptable salt, ester, solvate, or polymorph thereof, wherein:

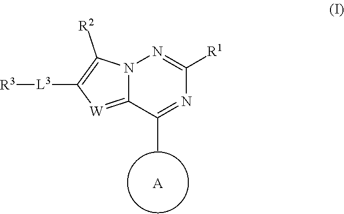

wherein:
   R¹ represents —H;
   R² represents —H;
   R³ represents 6,6-dimethyl-2,4-dioxo-3-azabicyclo[3.1.0]hexan-3-yl, —OH, ethoxy, or —NH₂;
   L³ represents —CH₂— or —C(═O)—;
   W represents C(—R⁴);
   R⁴ represents —H or —CH₃;
   and

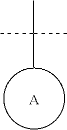

represents:
a)

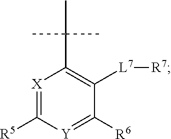

each X and Y, independently, represents C(—R^{C});
R^{C} represents —H;
R⁵ represents —Cl;
R⁶ represents methyl;
R⁷ represents piperidin-3-yl, morpholin-2-yl, 6-methylpiperidin-3-yl, piperidin-4-yl, 2-methylpiperazin-1-yl, 2-carboxypyrrolidin-4-yl, 3,3-difluoroazetidin-1-yl, 2-aminoethyl, 2,6-dimethylpiperazin-1-yl, 4-fluoropyrrolidin-3-yl, 5-methylpyrrolidin-3-yl, or 3-amino-4-methylpyrrolidin-1-yl; and
L⁷ represents —CH₂—, —O—, or —C(═O)—:
or:
b)

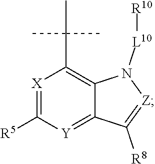

each X and Y, independently, represents C(—R^{C});
R^{C} represents —H;
R⁵ represents —Cl or —CF₃;
R⁸ represents —H;
Z represents C(—R⁹);
R⁹ represents —H;
R¹⁰ represents pyrrolidin-3-yl, morpholin-2-yl, 5,5-difluoropiperidin-3-yl, 4-fluoropiperidin-4-yl, 4-cyanopiperidin-4-yl, or 4-hydroxypiperidin-4-yl; and
L¹⁰ represents a single bond or —CH₂—.

In some preferred embodiments, the invention provides a compound of Formula (I), as defined above, or a pharmaceutically acceptable salt, ester, solvate, or polymorph thereof, wherein:
either:
   a) R⁷ represents (S)-piperidin-3-yl, (R)-morpholin-2-yl, (3R,6R)-6-methylpiperidin-3-yl, piperidin-4-yl, (S)-2-methylpiperazin-1-yl, (2S,4S)-2-carboxypyrrolidin-4-yl, 3,3-difluoroazetidin-1-yl, 2-aminoethyl, (2S,6S)-2,6-dimethylpiperazin-1-yl, (3R,4S)-4-fluoropyrrolidin-3-yl, (3R,5R)-5-methylpyrrolidin-3-yl, or (3S,4S)-3-amino-4-methylpyrrolidin-1-yl; and
      L⁷ represents —CH₂— or —O—;
or:
   b) R¹⁰ represents (R)-pyrrolidin-3-yl, (S)-pyrrolidin-3-yl, 5,5-difluoropiperidin-3-yl, 4-fluoropiperidin-4-yl, 4-cyanopiperidin-4-yl, or 4-hydroxypiperidin-4-yl; and
      L¹⁰ represents —CH₂—.

In some preferred embodiments, the invention provides a compound of Formula (I), as defined above, or a pharmaceutically acceptable salt, ester, solvate, or polymorph thereof, wherein:
either:
   a) R⁷ represents (S)-piperidin-3-yl, (R)-morpholin-2-yl, (S)-2-methylpiperazin-1-yl, (2S,6S)-2,6-dimethylpiperazin-1-yl, or (3R,4S)-4-fluoropyrrolidin-3-yl;
or:
   b) R¹⁰ represents 4-fluoropiperidin-4-yl.

In some preferred embodiments, the invention provides a compound of Formula (I), as defined above, or a pharmaceutically acceptable salt, ester, solvate, or polymorph thereof, wherein:
R³ represents 6,6-dimethyl-2,4-dioxo-3-azabicyclo[3.1.0]hexan-3-yl; and
L³ represents —CH₂—.

In some preferred embodiments, the invention provides a compound of Formula (I), as defined above, or a pharmaceutically acceptable salt, ester, solvate, or polymorph thereof, wherein:
W represents C(—R⁴); and
R⁴ represents —H.

In some preferred embodiments, the invention provides a compound of Formula (I), as defined above, or a pharmaceutically acceptable salt, ester, solvate, or polymorph thereof, wherein:
R⁵ represents —Cl.

In some preferred embodiments, the invention provides a compound of Formula (I), as defined above, or a pharmaceutically acceptable salt, ester, solvate, or polymorph thereof, wherein:
R⁶ represents methyl.

In some preferred embodiments, the compound according to the invention is:
3-((4-(5-chloro-3-methyl-2-(((S)-piperidin-3-yl)oxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((R)-morpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((3R,6R)-6-methylpiperidin-3-yl)oxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(piperidin-4-yloxy)phenyl)pyrrolo[2,1-f][1,2,4]triazin-6-yl)methyl)-6,6-dimethyl-3-azabicyclo[3.1.0]hexane-2,4-dione;
6,6-dimethyl-3-((4-(1-(pyrrolidin-3-yl)-5-(trifluoromethyl)-1H-indol-7-yl)pyrrolo[2,1-f][1,2,4]triazin-6-yl)methyl)-3-azabicyclo[3.1.0]hexane-2,4-dione;
3-((4-(5-chloro-3-methyl-2-(((S)-2-methylpiperazin-1-yl)methyl)phenyl)pyrrolo[2,1-f][1,2,4]triazin-6-yl)methyl)-6,6-dimethyl-3-azabicyclo[3.1.0]hexane-2,4-dione;
(2S,4S)-4-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenoxy)pyrrolidine-2-carboxylic acid;
3-((4-(5-chloro-2-(3,3-difluoroazetidine-1-carbonyl)-3-methylphenyl)pyrrolo[2,1-f][1,2,4]triazin-6-yl)methyl)-6,6-dimethyl-3-azabicyclo[3.1.0]hexane-2,4-dione;
3-((4-(2-(2-aminoethoxy)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-1-((4-fluoropiperidin-4-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(5-chloro-2-(((2S,6S)-2,6-dimethylpiperazin-1-yl)methyl)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((S)-piperidin-3-yl)oxy)phenyl)-5-methylpyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((R)-morpholin-2-yl)methyl)phenyl)-5-methylpyrrolo[2,1-f][1,2,4]triazin-6-yl)methyl)-6,6-dimethyl-3-azabicyclo[3.1.0]hexane-2,4-dione;
3-((4-(5-chloro-2-(((3R,4S)-4-fluoropyrrolidin-3-yl)oxy)-3-methylphenyl)pyrrolo[2,1-f][1,2,4]triazin-6-yl)methyl)-6,6-dimethyl-3-azabicyclo[3.1.0]hexane-2,4-dione;
ethyl (S)-4-(5-chloro-3-methyl-2-(piperidin-3-yloxy)phenyl)pyrrolo[2,1-f][1,2,4]triazine-6-carboxylate;
(S)-4-(5-chloro-3-methyl-2-(piperidin-3-yloxy)phenyl)pyrrolo[2,1-f][1,2,4]triazine-6-carboxylic acid;
(S)-(4-(5-chloro-3-methyl-2-(piperidin-3-yloxy)phenyl)pyrrolo[2,1-f][1,2,4]triazin-6-yl)methanol;
(S)-4-(5-chloro-3-methyl-2-(piperidin-3-yloxy)phenyl)pyrrolo[2,1-f][1,2,4]triazine-6-carboxamide;
3-((4-(1-((4-hydroxypiperidin-4-yl)methyl)-5-(trifluoromethyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((3R,5R)-5-methylpyrrolidin-3-yl)oxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-1-((4-fluoropiperidin-4-yl)methyl)-1H-indol-7-yl)-5-methylpyrrolo[2,1-f][1,2,4]triazin-6-yl)methyl)-6,6-dimethyl-3-azabicyclo[3.1.0]hexane-2,4-dione;
3-((4-(1-((4-fluoropiperidin-4-yl)methyl)-5-(trifluoromethyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(2-((3S,4S)-3-amino-4-methylpyrrolidine-1-carbonyl)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-1-(morpholin-2-ylmethyl)-1H-indol-7-yl)pyrrolo[2,1-f][1,2,4]triazin-6-yl)methyl)-6,6-dimethyl-3-azabicyclo[3.1.0]hexane-2,4-dione;
4-((5-chloro-7-(6-((6,6-dimethyl-2,4-dioxo-3-azabicyclo[3.1.0]hexan-3-yl)methyl)pyrrolo[2,1-f][1,2,4]triazin-4-yl)-1H-indol-1-yl)methyl)piperidine-4-carbonitrile; or
3-((4-(5-chloro-1-((5,5-difluoropiperidin-3-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
   or a tautomer, stereoisomer, a racemic or scalemic mixture of stereoisomers, a pharmaceutically acceptable salt, ester, solvate, or polymorph thereof.

In some preferred embodiments, the invention provides a compound of Formula (I), as defined above, or a tautomer, stereoisomer, a racemic or scalemic mixture of stereoisomers, a pharmaceutically acceptable salt, ester, solvate, or polymorph thereof, wherein:

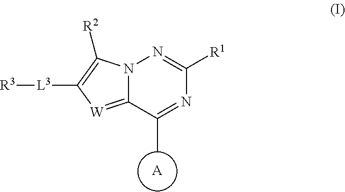

wherein:
   R¹ represents —H;
   R² represents —H;
   R³ represents 6,6-dimethyl-2,4-dioxo-3-azabicyclo[3.1.0]hexan-3-yl, ethoxy, —OH, —NH₂, 2,4-dioxo-dihydropyrimidin-3-yl, 1-methyl-2,4-dioxo-dihydropyrimidin-3-yl, 1-methyl-2,4-dioxo-pyrimidin-3-yl, 1-(methyl-d₃)-2,4-dioxo-pyrimidin-3-yl, 1-ethyl-2,4-dioxo-pyrimidin-3-yl, 1-(2-fluoroethyl)-2,4-dioxo-pyrimidin-3-yl, 1-(2,2-difluoroethyl)-2,4-dioxo-pyrimidin-3-yl, 1-(2,2,2-trifluoroethyl)-2,4-dioxo-pyrimidin-3-yl, 1-isopropyl-2,4-dioxo-pyrimidin-3-yl, 1-cyclopropyl-2,4-dioxo-pyrimidin-3-yl, 5-fluoro-1-methyl-2,4-dioxo-pyrimidin-3-yl, 1,5-dimethyl-2,4-dioxo-pyrimidin-3-yl, 1-carboxymethyl-2,4-dioxo-dihydropyrimidin-3-yl, 1-methyl-2,4-dioxo-imidazolidin-3-yl, or 4-methyl-2,6-dioxo-piperazin-1-yl;
   L³ represents —CH₂— or —C(═O)—;
   W represents C(—R⁴);
   R⁴ represents —H or —CH₃;
   and

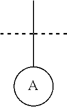

represents:

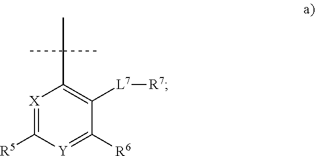

X represents C(—R^{C});
Y represents C(—R^{C}) or N;
R^{C} represents —H;
R⁵ represents —F, —Cl, or —CF₃;
R⁶ represents —H, —F, —Cl, or —CH₃;
R⁷ represents piperidin-3-yl, morpholin-2-yl, 6-methylpiperidin-3-yl, piperidin-4-yl, 2-methylpiperazin-1-yl, 2-carboxypyrrolidin-4-yl, 3,3-difluoroazetidin-1-yl, 2-aminoethyl, 2,6-dimethylpiperazin-1-yl, 4-fluoropyrrolidin-3-yl, 5-methylpyrrolidin-3-yl, 3-amino-4-methylpyrrolidin-1-yl, 6-methylmorpholin-2-yl, 5,5-difluoropiperidin-3-yl, 6,6-difluoro-1,4-oxepan-2-yl, 5-methylmorpholin-2-yl, 6-fluoromethylmorpholin-2-yl, 4-oxa-7-azaspiro[2.5]octan-5-yl, 6,6-dimethylmorpholin-2-yl, 4-methylpiperidin-4-yl, 7-oxa-4-azaspiro[2.5]octan-6-yl, 2,6,6-trimethylmorpholin-2-yl, 3-methylpiperidin-3-yl, 4-fluoropiperidin-4-yl, 1,2,3,6-tetrahydropyridin-4-yl, piperazin-1-yl, 3-aminopyrrolidin-1-yl, hexahydropyrrolo[3,4-b]pyrrol-5(1H)-yl, 1,7-diazaspiro[4,4]nonan-7-yl, hexahydropyrrolo[3,4-c]pyrrol-2(1H)-yl, 2,7-diazaspiro[3.5]nonan-7-yl, 4-methylmorpholin-2-yl, or 3-(methylamino)piperidin-1-yl; and
L⁷ represents a single bond, —CH₂—, —O—, —O—CH₂—, —O—CH(—CH₃)— or —C(═O)—.

In some preferred embodiments, the compound according to the invention is:
3-((4-(5-chloro-3-methyl-2-(((S)-piperidin-3-yl)oxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((R)-morpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((3R,6R)-6-methylpiperidin-3-yl)oxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(piperidin-4-yloxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((S)-2-methylpiperazin-1-yl)methyl)phenyl)pyrrolo[2,1-f][1,2,4]triazin-6-yl)methyl)-6,6-dimethyl-3-azabicyclo[3.1.0]hexane-2,4-dione;
(2S,4S)-4-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenoxy)pyrrolidine-2-carboxylic acid;
3-((4-(5-chloro-2-(3,3-difluoroazetidine-1-carbonyl)-3-methylphenyl)pyrrolo[2,1-f][1,2,4]triazin-6-yl)methyl)-6,6-dimethyl-3-azabicyclo[3.1.0]hexane-2,4-dione;
3-((4-(2-(2-aminoethoxy)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-2-(((2S,6S)-2,6-dimethylpiperazin-1-yl)methyl)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((S)-piperidin-3-yl)oxy)phenyl)-5-methylpyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((R)-morpholin-2-yl)methyl)phenyl)-5-methylpyrrolo[2,1-f][1,2,4]triazin-6-yl)methyl)-6,6-dimethyl-3-azabicyclo[3.1.0]hexane-2,4-dione;
3-((4-(5-chloro-2-(((3R,4S)-4-fluoropyrrolidin-3-yl)oxy)-3-methylphenyl)pyrrolo[2,1-f][1,2,4]triazin-6-yl)methyl)-6,6-dimethyl-3-azabicyclo[3.1.0]hexane-2,4-dione;
ethyl (S)-4-(5-chloro-3-methyl-2-(piperidin-3-yloxy)phenyl)pyrrolo[2,1-f][1,2,4]triazine-6-carboxylate;
(S)-4-(5-chloro-3-methyl-2-(piperidin-3-yloxy)phenyl)pyrrolo[2,1-f][1,2,4]triazine-6-carboxylic acid;
(S)-(4-(5-chloro-3-methyl-2-(piperidin-3-yloxy)phenyl)pyrrolo[2,1-f][1,2,4]triazin-6-yl)methanol;
(S)-4-(5-chloro-3-methyl-2-(piperidin-3-yloxy)phenyl)pyrrolo[2,1-f][1,2,4]triazine-6-carboxamide;
3-((4-(5-chloro-3-methyl-2-(((3S,5S)-5-methylpyrrolidin-3-yl)oxy)phenyl)pyrrolo[2,1-f][1,2,4]triazin-6-yl)methyl)-6,6-dimethyl-3-azabicyclo[3.1.0]hexane-2,4-dione;
3-((4-(2-((3S,4S)-3-amino-4-methylpyrrolidine-1-carbonyl)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((6S)-6-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-(((S)-5,5-difluoropiperidin-3-yl)oxy)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-2-((6,6-difluoro-1,4-oxazepan-2-yl)methyl)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((5R)-5-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((5R)-5-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-((6-(fluoromethyl)morpholin-2-yl)methyl)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-2-((6-(fluoromethyl)morpholin-2-yl)methyl)-3-methylphenyl)pyrrolo[2,1-f][1,2,4]triazin-6-yl)methyl)-6,6-dimethyl-3-azabicyclo[3.1.0]hexane-2,4-dione;
3-((4-(2-((4-oxa-7-azaspiro[2.5]octan-5-yl)methyl)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(piperidin-4-ylmethoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((5S)-5-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-((6,6-dimethylmorpholin-2-yl)methyl)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((R)-morpholin-2-yl)methoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((R)-piperidin-3-yl)methoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(1-(piperidin-4-yl)ethoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(1-(piperidin-4-yl)ethoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((4-methylpiperidin-4-yl)methoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione;
3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-(2,2,2-trifluoroethyl)pyrimidine-2,4(1H,3H)-dione;
3-((4-(5-chloro-3-methyl-2-(morpholin-2-ylmethyl)phenyl)pyrrolo[2,1-f][1,2,4]triazin-6-yl)methyl)-1-(2,2,2-trifluoroethyl)pyrimidine-2,4(1H,3H)-dione;
3-((4-(2-((7-oxa-4-azaspiro[2.5]octan-6-yl)methyl)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((2,6,6-trimethylmorpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((piperidin-4-ylmethyl)amino)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((3-methylpiperidin-3-yl)methoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((3-methylpiperidin-3-yl)methoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((R)-1-((S)-morpholin-2-yl)ethoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((R)-1-((S)-morpholin-2-yl)ethoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((4-methylpiperidin-4-yl)methyl)amino)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((piperidin-3-ylmethyl)amino)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-(((4-fluoropiperidin-4-yl)methyl)amino)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((((S)-morpholin-2-yl)methyl)amino)phenyl)pyrrolo[2,1-f][1,2,4]triazin-6-yl)methyl)-6,6-dimethyl-3-azabicyclo[3.1.0]hexane-2,4-dione;
(R)-3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-(2,2,2-trifluoroethyl)pyrimidine-2,4(1H,3H)-dione;
3-((4-(5-chloro-3-methyl-2-(1,2,3,6-tetrahydropyridin-4-yl)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-(piperazin-1-yl)phenyl)pyrrolo[2,1-f][1,2,4]triazin-6-yl)methyl)-6,6-dimethyl-3-azabicyclo[3.1.0]hexane-2,4-dione;
3-((4-(2-((S)-3-aminopyrrolidin-1-yl)-5-chlorophenyl)pyrrolo[2,1-f][1,2,4]triazin-6-yl)methyl)-6,6-dimethyl-3-azabicyclo[3.1.0]hexane-2,4-dione;
3-((4-(2-((R)-3-aminopyrrolidin-1-yl)-5-chlorophenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(1,2,3,6-tetrahydropyridin-4-yl)phenyl)pyrrolo[2,1-f][1,2,4]triazin-6-yl)methyl)-1-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-isopropylpyrimidine-2,4(1H,3H)-dione;
3-((4-(5-chloro-2-((3aR,6aR)-hexahydropyrrolo[3,4-b]pyrrol-5(1H)-yl)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-(1,7-diazaspiro[4.4]nonan-7-yl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-(2,2-difluoroethyl)pyrimidine-2,4(1H,3H)-dione;
3-((4-(5-chloro-2-((3aS,6aS)-hexahydropyrrolo[3,4-b]pyrrol-5(1H)-yl)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-(hexahydropyrrolo[3,4-c]pyrrol-2(1H)-yl)phenyl)pyrrolo[2,1-f][1,2,4]triazin-6-yl)methyl)-6,6-dimethyl-3-azabicyclo[3.1.0]hexane-2,4-dione;
(R)-1-((4-(5-chloro-3-methyl-2-(morpholin-2-ylmethyl)phenyl)pyrrolo[2,1-f][1,2,4]triazin-6-yl)methyl)-5-fluoro-3-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-methylimidazolidine-2,4-dione;
(S)-3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-(2-fluoroethyl)pyrimidine-2,4(1H,3H)-dione;
3-((4-(2-((2,7-diazaspiro[3.5]nonan-7-yl)methyl)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-fluoro-2-((4-methylmorpholin-2-yl)methyl)phenyl)pyrrolo[2,1-f][1,2,4]triazin-6-yl)methyl)-1-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-ethylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-(methyl-d₃)pyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-cyclopropylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-cyclopropylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-5-fluoro-1-methylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-methyldihydropyrimidine-2,4(1H,3H)-dione;
3-((4-(5-chloro-3-methyl-2-(((5S)-5-methylmorpholin-2-yl)methyl)phenyl)pyrrolo[2,1-f][1,2,4]triazin-6-yl)methyl)-1-methylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-isopropylpyrimidine-2,4(1H,3H)-dione;
3-((4-(5-chloro-2-((S)-3-(methylamino)piperidin-1-yl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((5S)-5-methylmorpholin-2-yl)methyl)phenyl)pyrrolo[2,1-f][1,2,4]triazin-6-yl)methyl)-1-isopropylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-ethylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1,5-dimethylpyrimidine-2,4(1H,3H)-dione;
3-((4-(5-chloro-3-fluoro-2-(morpholin-2-ylmethyl)phenyl)pyrrolo[2,1-f][1,2,4]triazin-6-yl)methyl)-1-methylpyrimidine-2,4(1H,3H)-dione;
1-((4-(5-chloro-3-methyl-2-(((6S)-6-methylmorpholin-2-yl)methyl)phenyl)pyrrolo[2,1-f][1,2,4]triazin-6-yl)methyl)-4-methylpiperazine-2,6-dione;
3-((4-(3,5-dichloro-2-(morpholin-2-ylmethyl)phenyl)pyrrolo[2,1-f][1,2,4]triazin-6-yl)methyl)-1-methylpyrimidine-2,4(1H,3H)-dione;
1-((4-(5-chloro-3-fluoro-2-(((6S)-6-methylmorpholin-2-yl)methyl)phenyl)pyrrolo[2,1-f][1,2,4]triazin-6-yl)methyl)-4-methylpiperazine-2,6-dione;
3-((4-(5-chloro-3-fluoro-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)pyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)pyrimidine-2,4(1H,3H)-dione;
(R)-2-(3-((4-(5-chloro-3-methyl-2-(morpholin-2-ylmethyl)phenyl)pyrrolo[2,1-f][1,2,4]triazin-6-yl)methyl)-2,4-dioxo-3,4-dihydropyrimidin-1(2H)-yl)acetic acid;
3-((4-(3,5-dichloro-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione;
3-((4-(5-fluoro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione; or
1-methyl-3-((4-(2-methyl-3-(piperidin-3-yloxy)-6-(trifluoromethyl)pyridin-4-yl)pyrrolo[2,1-f][1,2,4]triazin-6-yl)methyl)pyrimidine-2,4(1H,3H)-dione;
   or a tautomer, stereoisomer, a racemic or scalemic mixture of stereoisomers, a pharmaceutically acceptable salt, ester, solvate, or polymorph thereof.

In some more preferred embodiments, the compound according to the invention is:
3-((4-(5-chloro-3-methyl-2-(((R)-morpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-isopropylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-(methyl-d₃)pyrimidine-2,4(1H,3H)-dione; or
3-((4-(5-chloro-3-methyl-2-(((6S)-6-methylmorpholin-2-yl)methyl)phenyl)pyrrolo[2,1-f][1,2,4]triazin-6-yl)methyl)-1-isopropylpyrimidine-2,4(1H,3H)-dione;
   or a tautomer, stereoisomer, a racemic or scalemic mixture of stereoisomers, a pharmaceutically acceptable salt, ester, solvate, or polymorph thereof.

In some preferred embodiments, the invention provides a compound of Formula (I), as defined above, or a tautomer, stereoisomer, a racemic or scalemic mixture of stereoisomers, a pharmaceutically acceptable salt, ester, solvate, or polymorph thereof, wherein:

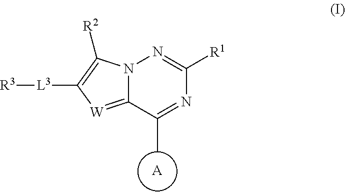

wherein:
   R¹ represents —H or —Cl;
   R² represents —H or —F;
   R³ represents 6,6-dimethyl-2,4-dioxo-3-azabicyclo[3.1.0]hexan-3-yl, 1,3-dioxo-tetrahydropyrrolo[1,2-a]pyrazin-2-yl, 5,5-dimethyl-2,4-dioxo-imidazolidin-3-yl, 2,4-dioxo-imidazolidin-3-yl, 1-methyl-2,4-dioxo-pyrimidin-3-yl, 1-(2,2,2-trifluoroethyl)-2,4-dioxo-pyrimidin-3-yl, 2,4-dioxo-pyrimidin-3-yl, 1-isopropyl-2,4-dioxo-pyrimidin-3-yl, or 1-(methyl-d₃)-2,4-dioxo-pyrimidin-3-yl;
   L³ represents —CH₂—;
   W represents C(—R⁴);
   R⁴ represents —H or —CH₃;
   and

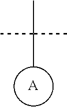

represents:
b)

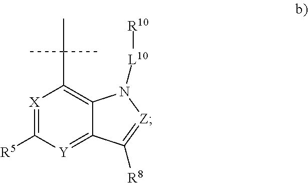

each X and Y, independently, represents C(—R^{C});
R^{C} represents —H;
R⁵ represents —F, —Cl, —CF₃, —NO₂, carboxy, or methoxycarbonyl;
R⁸ represents —H or —F;
Z represents C(—R⁹) or N;
if exists, R⁹ represents —H;
R¹⁰ represents pyrrolidin-3-yl, 4-fluoropiperidin-4-yl, 4-hydroxypiperidin-4-yl, morpholin-2-yl, 4-cyanopiperidin-4-yl, 5,5-difluoropiperidin-3-yl, or 5-fluoropiperidin-3-yl; and
L¹⁰ represents a single bond or —CH₂—.

In some preferred embodiments, the compound according to the invention is:
6,6-dimethyl-3-((4-(1-(pyrrolidin-3-yl)-5-(trifluoromethyl)-1H-indol-7-yl)pyrrolo[2,1-f][1,2,4]triazin-6-yl)methyl)-3-azabicyclo[3.1.0]hexane-2,4-dione;
3-((4-(5-chloro-1-((4-fluoropiperidin-4-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(1-((4-hydroxypiperidin-4-yl)methyl)-5-(trifluoromethyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(5-chloro-1-((4-fluoropiperidin-4-yl)methyl)-1H-indol-7-yl)-5-methylpyrrolo[2,1-f][1,2,4]triazin-6-yl)methyl)-6,6-dimethyl-3-azabicyclo[3.1.0]hexane-2,4-dione;
3-((4-(1-((4-fluoropiperidin-4-yl)methyl)-5-(trifluoromethyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(5-chloro-1-(morpholin-2-ylmethyl)-1H-indol-7-yl)pyrrolo[2,1-f][1,2,4]triazin-6-yl)methyl)-6,6-dimethyl-3-azabicyclo[3.1.0]hexane-2,4-dione;
4-((5-chloro-7-(6-((6,6-dimethyl-2,4-dioxo-3-azabicyclo[3.1.0]hexan-3-yl)methyl)pyrrolo[2,1-f][1,2,4]triazin-4-yl)-1H-indol-1-yl)methyl)piperidine-4-carbonitrile;
3-((4-(5-chloro-1-((5,5-difluoropiperidin-3-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(5-chloro-1-((3-fluoropiperidin-3-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(5-chloro-1-(((S)-morpholin-2-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
(S)-2-((4-(5-chloro-1-(((S)-morpholin-2-yl)methyl)-1H-indol-7-yl)pyrrolo[2,1-f][1,2,4]triazin-6-yl)methyl)tetrahydropyrrolo[1,2-a]pyrazine-1,3(2H,4H)-dione;
6,6-dimethyl-3-((4-(1-(((S)-morpholin-2-yl)methyl)-5-nitro-1H-indol-7-yl)pyrrolo[2,1-f][1,2,4]triazin-6-yl)methyl)-3-azabicyclo[3.1.0]hexane-2,4-dione;
3-((4-(5-fluoro-1-(((S)-morpholin-2-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
methyl 7-(6-((6,6-dimethyl-2,4-dioxo-3-azabicyclo[3.1.0]hexan-3-yl)methyl)pyrrolo[2,1-f][1,2,4]triazin-4-yl)-1-(((S)-morpholin-2-yl)methyl)-1H-indole-5-carboxylate;
6,6-dimethyl-3-((4-(1-(((S)-morpholin-2-yl)methyl)-5-(trifluoromethyl)-1H-indol-7-yl)pyrrolo[2,1-f][1,2,4]triazin-6-yl)methyl)-3-azabicyclo[3.1.0]hexane-2,4-dione;
(S)-3-((4-(5-chloro-1-(morpholin-2-ylmethyl)-1H-indol-7-yl)pyrrolo[2,1-f][1,2,4]triazin-6-yl)methyl)-5,5-dimethylimidazolidine-2,4-dione;
(S)-3-((4-(5-chloro-1-(morpholin-2-ylmethyl)-1H-indol-7-yl)pyrrolo[2,1-f][1,2,4]triazin-6-yl)methyl)imidazolidine-2,4-dione;
(S)-3-((4-(5-chloro-1-(morpholin-2-ylmethyl)-1H-indol-7-yl)pyrrolo[2,1-f][1,2,4]triazin-6-yl)methyl)-1-methylpyrimidine-2,4(1H,3H)-dione;
3-((4-(5-chloro-1-(((S)-morpholin-2-yl)methyl)-1H-indazol-7-yl)pyrrolo[2,1-f][1,2,4]triazin-6-yl)methyl)-6,6-dimethyl-3-azabicyclo[3.1.0]hexane-2,4-dione;
3-((4-(5-chloro-3-fluoro-1-(((S)-morpholin-2-yl)methyl)-1H-indazol-7-yl)pyrrolo[2,1-f][1,2,4]triazin-6-yl)methyl)-6,6-dimethyl-3-azabicyclo[3.1.0]hexane-2,4-dione;
7-(6-((6,6-dimethyl-2,4-dioxo-3-azabicyclo[3.1.0]hexan-3-yl)methyl)pyrrolo[2,1-f][1,2,4]triazin-4-yl)-1-(((S)-morpholin-2-yl)methyl)-1H-indole-5-carboxylic acid;
3-((4-(5-chloro-1-((4-fluoropiperidin-4-yl)methyl)-1H-indol-7-yl)pyrrolo[2,1-f][1,2,4]triazin-6-yl)methyl)-1-methylpyrimidine-2,4(1H,3H)-dione;
3-((4-(5-chloro-1-((4-fluoropiperidin-4-yl)methyl)-1H-indol-7-yl)pyrrolo[2,1-f][1,2,4]triazin-6-yl)methyl)-1-(2,2,2-trifluoroethyl)pyrimidine-2,4(1H,3H)-dione;
3-((2-chloro-4-(5-chloro-1-((4-fluoropiperidin-4-yl)methyl)-1H-indol-7-yl)pyrrolo[2,1-f][1,2,4]triazin-6-yl)methyl)pyrimidine-2,4(1H,3H)-dione;
3-((4-(5-chloro-1-((4-fluoropiperidin-4-yl)methyl)-1H-indol-7-yl)pyrrolo[2,1-f][1,2,4]triazin-6-yl)methyl)-1-isopropylpyrimidine-2,4(1H,3H)-dione;
3-((4-(5-chloro-1-((4-fluoropiperidin-4-yl)methyl)-1H-indol-7-yl)pyrrolo[2,1-f][1,2,4]triazin-6-yl)methyl)pyrimidine-2,4(1H,3H)-dione;
3-((4-(5-chloro-1-((4-fluoropiperidin-4-yl)methyl)-1H-indol-7-yl)pyrrolo[2,1-f][1,2,4]triazin-6-yl)methyl)-1-(methyl-d₃)pyrimidine-2,4(1H,3H)-dione; or 3-((4-(5-chloro-1-((4-fluoropiperidin-4-yl)methyl)-1H-indol-7-yl)-7-fluoropyrrolo[2,1-f][1,2,4]triazin-6-yl)methyl)-1-methylpyrimidine-2,4(1H,3H)-dione;
   or a tautomer, stereoisomer, a racemic or scalemic mixture of stereoisomers, a pharmaceutically acceptable salt, ester, solvate, or polymorph thereof.

In some more preferred embodiments, the compound according to the invention is:
3-((4-(5-chloro-1-((4-fluoropiperidin-4-yl)methyl)-1H-indol-7-yl)pyrrolo[2,1-f][1,2,4]triazin-6-yl)methyl)-1-methylpyrimidine-2,4(1H,3H)-dione;
3-((4-(5-chloro-1-((4-fluoropiperidin-4-yl)methyl)-1H-indol-7-yl)pyrrolo[2,1-f][1,2,4]triazin-6-yl)methyl)-1-isopropylpyrimidine-2,4(1H,3H)-dione; or
3-((4-(5-chloro-1-((4-fluoropiperidin-4-yl)methyl)-1H-indol-7-yl)pyrrolo[2,1-f][1,2,4]triazin-6-yl)methyl)-1-(methyl-d₃)pyrimidine-2,4(1H,3H)-dione;
   or a tautomer, stereoisomer, a racemic or scalemic mixture of stereoisomers, a pharmaceutically acceptable salt, ester, solvate, or polymorph thereof.

In some preferred embodiments, the invention provides a compound of Formula (I), as defined above, or a tautomer, stereoisomer, a racemic or scalemic mixture of stereoisomers, a pharmaceutically acceptable salt, ester, solvate, or polymorph thereof, wherein:

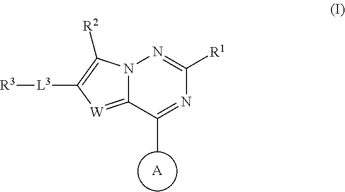

wherein:
   R¹ represents —H;
   R² represents —H;
   R³ represents 6,6-dimethyl-2,4-dioxo-3-azabicyclo[3.1.0]hexan-3-yl;
   L³ represents —CH₂—;
   W represents C(—R⁴);
   R⁴ represents —H;
   and

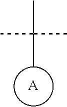

represents:

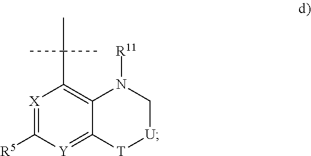

wherein each X and Y, independently, represents C(—R^{C});
each R^{C} represents —H;
R⁵ represents —Cl;
R¹¹ represents pyrrolidin-3-yl, piperidin-4-yl, morpholin-2-yl, or azetidin-3-yl; and
either T represents CH₂ or O, and U represents —CH₂— or CH(—CH₃),
or -T-U— represents

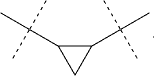

In some preferred embodiments, the compound according to the invention is:
3-((4-(6-chloro-1-(pyrrolidin-3-yl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(6-chloro-1-(piperidin-4-yl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(7-chloro-4-(pyrrolidin-3-yl)-3,4-dihydro-2H-benzo[b][1,4]oxazin-5-yl)pyrrolo[2,1-f][1,2,4]triazin-6-yl)methyl)-6,6-dimethyl-3-azabicyclo[3.1.0]hexane-2,4-dione;
3-((4-(6-chloro-1-((4-fluoropiperidin-4-yl)methyl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(6-chloro-1-(pyrrolidin-3-yl)-1,2,3,4-tetrahydroquinolin-8-yl)pyrrolo[2,1-f][1,2,4]triazin-6-yl)methyl)-1-(2,2,2-trifluoroethyl)pyrimidine-2,4(1H,3H)-dione;
3-((4-(6-chloro-1-(((S)-morpholin-2-yl)methyl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(6-chloro-1-(pyrrolidin-3-yl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(6-chloro-1-(pyrrolidin-3-yl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(1-(azetidin-3-yl)-6-chloro-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(6-chloro-3-(pyrrolidin-3-yl)-1a,2,3,7b-tetrahydro-1H-cyclopropa[c]quinolin-4-yl)pyrrolo[2,1-f][1,2,4]triazin-6-yl)methyl)-6,6-dimethyl-3-azabicyclo[3.1.0]hexane-2,4-dione;
3-((4-((2R)-7-chloro-2-methyl-4-(pyrrolidin-3-yl)-3,4-dihydro-2H-benzo[b][1,4]oxazin-5-yl)pyrrolo[2,1-f][1,2,4]triazin-6-yl)methyl)-6,6-dimethyl-3-azabicyclo[3.1.0]hexane-2,4-dione; or
3-((4-((2S)-7-chloro-2-methyl-4-(pyrrolidin-3-yl)-3,4-dihydro-2H-benzo[b][1,4]oxazin-5-yl)pyrrolo[2,1-f][1,2,4]triazin-6-yl)methyl)-6,6-dimethyl-3-azabicyclo[3.1.0]hexane-2,4-dione;
   or a tautomer, stereoisomer, a racemic or scalemic mixture of stereoisomers, a pharmaceutically acceptable salt, ester, solvate, or polymorph thereof.

The following representative structures of compounds of Formula (I), in the form of acid addition salts thereof, are disclosed herein:

| Ex. No. | Structure | IUPAC Name |
|---|---|---|
| 1. | 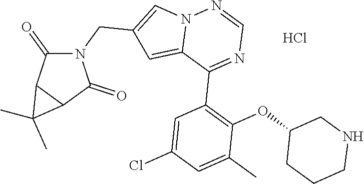 [SMILES: Cc1cc(Cl)cc(-c2ncnn3cc(CN4C(=O)C5C(C4=O)C5(C)C)cc23)c1O[C@H]1CCCNC1 ; Cl] | 3-((4-(5-chloro-3-methyl-2-(((S)- piperidin-3- yl)oxy)phenyl)pyrrolo[2,1- f][1,2,4]triazin-6-yl)methyl)-6,6- dimethyl-3-azabicyclo[3.1.0]hexane- 2,4-dione hydrochloride |
| | A single enantiomer | |
| 2. | 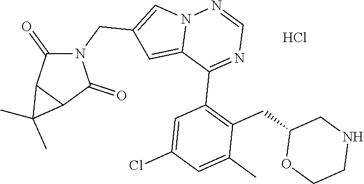 [SMILES: Cc1cc(Cl)cc(-c2ncnn3cc(CN4C(=O)C5C(C4=O)C5(C)C)cc23)c1C[C@@H]1CNCCO1 ; Cl] | 3-((4-(5-chloro-3-methyl-2-(((R)- morpholin-2- yl)methyl)phenyl)pyrrolo[2,1- f][1,2,4]triazin-6-yl)methyl)-6,6- dimethyl-3-azabicyclo[3.1.0]hexane- 2,4-dione hydrochloride |
| | A single enantiomer | |
| 3. | 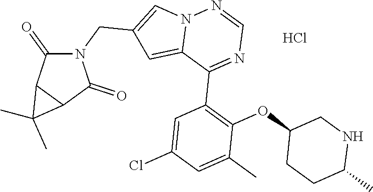 | 3-((4-(5-chloro-3-methyl-2- (((3R,6R)-6-methylpiperidin-3- yl)oxy)phenyl)pyrrolo[2,1- f][1,2,4]triazin-6-yl)methyl)-6,6- dimethyl-3-azabicyclo[3.1.0]hexane- 2,4-dione hydrochloride |
| | A single diastereoisomer | |
| 4. | 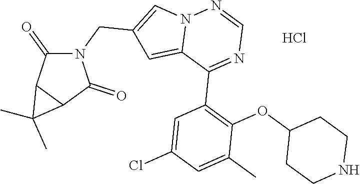 [SMILES: Cc1cc(Cl)cc(-c2ncnn3cc(CN4C(=O)C5C(C4=O)C5(C)C)cc23)c1OC1CCNCC1 ; Cl] | 3-((4-(5-chloro-3-methyl-2- (piperidin-4- yloxy)phenyl)pyrrolo[2,1- f][1,2,4]triazin-6-yl)methyl)-6,6- dimethyl-3-azabicyclo[3.1.0]hexane- 2,4-dione hydrochloride |
| 5. | 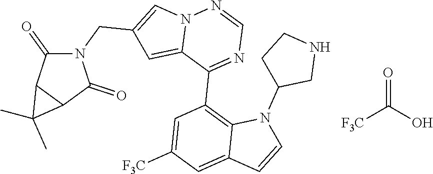 [SMILES: CC1(C)C2C(=O)N(Cc3cc4c(-c5cc(C(F)(F)F)cc6ccn(C7CCNC7)c56)ncnn4c3)C(=O)C21 ; O=C(O)C(F)(F)F] | 6,6-dimethyl-3-((4-(1-(pyrrolidin-3- yl)-5-(trifluoromethyl)-1H-indol-7- yl)pyrrolo[2,1-f][1,2,4]triazin-6- yl)methyl)-3- azabicyclo[3.1.0]hexane-2,4-dione 2,2,2-trifluoroacetate |
| | A racemate | |
| 6. | 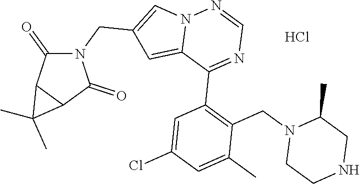 [SMILES: Cc1cc(Cl)cc(-c2ncnn3cc(CN4C(=O)C5C(C4=O)C5(C)C)cc23)c1CN1CCNC[C@@H]1C ; Cl] | 3-((4-(5-chloro-3-methyl-2-(((S)-2- methylpiperazin-1- yl)methyl)phenyl)pyrrolo[2,1- f][1,2,4]triazin-6-yl)methyl)-6,6- dimethyl-3-azabicyclo[3.1.0]hexane- 2,4-dione hydrochloride |
| | A single enantiomer | |
| 7. | 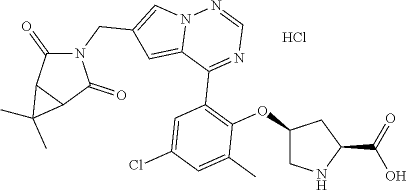 [SMILES: Cc1cc(Cl)cc(-c2ncnn3cc(CN4C(=O)C5C(C4=O)C5(C)C)cc23)c1O[C@@H]1CN[C@H](C(=O)O)C1 ; Cl] | (2S,4S)-4-(4-chloro-2-(6-((6,6- dimethyl-2,4-dioxo-3- azabicyclo[3.1.0]hexan-3- yl)methyl)pyrrolo[2,1- f][1,2,4]triazin-4-yl)-6- methylphenoxy)pyrrolidine-2- carboxylic acid hydrochloride |
| | A single diastereoisomer | |
| 8. | 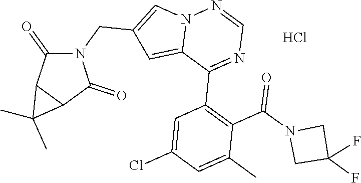 [SMILES: Cc1cc(Cl)cc(-c2ncnn3cc(CN4C(=O)C5C(C4=O)C5(C)C)cc23)c1C(=O)N1CC(F)(F)C1 ; Cl] | 3-((4-(5-chloro-2-(3,3- difluoroazetidine-1-carbonyl)-3- methylphenyl)pyrrolo[2,1- f][1,2,4]triazin-6-yl)methyl)-6,6- dimethyl-3-azabicyclo[3.1.0]hexane- 2,4-dione hydrochloride |
| 9. | 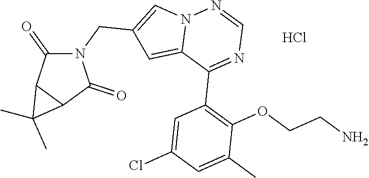 [SMILES: Cc1cc(Cl)cc(-c2ncnn3cc(CN4C(=O)C5C(C4=O)C5(C)C)cc23)c1OCCN ; Cl] | 3-((4-(2-(2-aminoethoxy)-5-chloro- 3-methylphenyl)pyrrolo[2,1- f][1,2,4]triazin-6-yl)methyl)-6,6- dimethyl-3-azabicyclo[3.1.0]hexane- 2,4-dione hydrochloride |
| 10. | 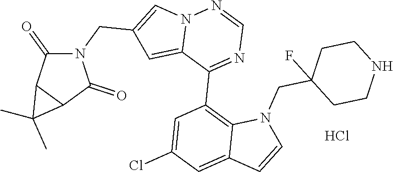 [SMILES: CC1(C)C2C(=O)N(Cc3cc4c(-c5cc(Cl)cc6ccn(CC7(F)CCNCC7)c56)ncnn4c3)C(=O)C21 ; Cl] | 3-((4-(5-chloro-1-((4- fluoropiperidin-4-yl)methyl)-1H- indol-7-yl)pyrrolo[2,1- f][1,2,4]triazin-6-yl)methyl)-6,6- dimethyl-3-azabicyclo[3.1.0]hexane- 2,4-dione hydrochloride |
| 11. | 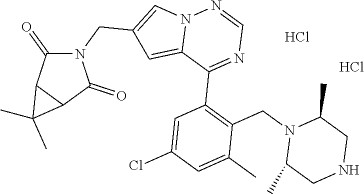 [SMILES: Cc1cc(Cl)cc(-c2ncnn3cc(CN4C(=O)C5C(C4=O)C5(C)C)cc23)c1CN1[C@@H](C)CNC[C@@H]1C ; Cl ; Cl] | 3-((4-(5-chloro-2-(((2S,6S)-2,6- dimethylpiperazin-1-yl)methyl)-3- methylphenyl)pyrrolo[2,1- f][1,2,4]triazin-6-yl)methyl)-6,6- dimethyl-3-azabicyclo[3.1.0]hexane- 2,4-dione dihydrochloride |
| | A single diastereoisomer | |
| 12. | 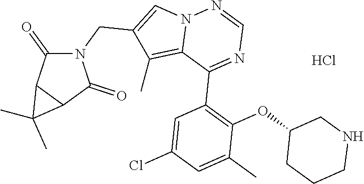 [SMILES: Cc1cc(Cl)cc(-c2ncnn3cc(CN4C(=O)C5C(C4=O)C5(C)C)c(C)c23)c1O[C@H]1CCCNC1 ; Cl] | 3-((4-(5-chloro-3-methyl-2-(((S)- piperidin-3-yl)oxy)phenyl)-5- methylpyrrolo[2,1-f][1,2,4]triazin-6- yl)methyl)-6,6-dimethyl-3- azabicyclo[3.1.0]hexane-2,4-dione hydrochloride |
| | A mixture of atropoisomers | |
| 13. | 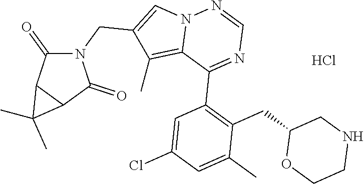 [SMILES: Cc1cc(Cl)cc(-c2ncnn3cc(CN4C(=O)C5C(C4=O)C5(C)C)c(C)c23)c1C[C@@H]1CNCCO1 ; Cl] | 3-((4-(5-chloro-3-methyl-2-(((R)- morpholin-2-yl)methyl)phenyl)-5- methylpyrrolo[2,1-f][1,2,4]triazin-6- yl)methyl)-6,6-dimethyl-3- azabicyclo[3.1.0]hexane-2,4-dione hydrochloride |
| | A mixture of atropoisomers | |
| 14. | 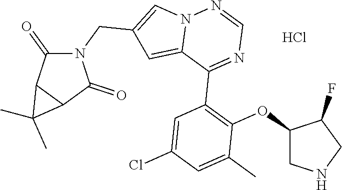 [SMILES: Cc1cc(Cl)cc(-c2ncnn3cc(CN4C(=O)C5C(C4=O)C5(C)C)cc23)c1O[C@@H]1CNC[C@@H]1F ; Cl] | 3-((4-(5-chloro-2-(((3R,4S)-4- fluoropyrrolidin-3-yl)oxy)-3- methylphenyl)pyrrolo[2,1- f][1,2,4]triazin-6-yl)methyl)-6,6- dimethyl-3-azabicyclo[3.1.0]hexane- 2,4-dione hydrochloride |
| | A single diastereoisomer | |
| 15. | 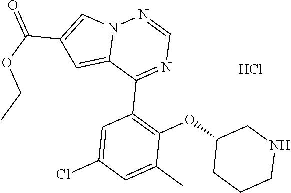 [SMILES: CCOC(=O)c1cc2c(-c3cc(Cl)cc(C)c3O[C@H]3CCCNC3)ncnn2c1 ; Cl] | ethyl (S)-4-(5-chloro-3-methyl-2- (piperidin-3- yloxy)phenyl)pyrrolo[2,1- f][1,2,4]triazine-6-carboxylate hydrochloride |
| | A single enantiomer | |
| 16. | 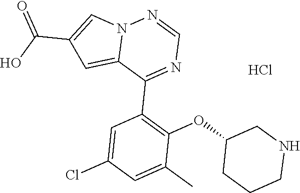 [SMILES: Cc1cc(Cl)cc(-c2ncnn3cc(C(=O)O)cc23)c1O[C@H]1CCCNC1 ; Cl] | (S)-4-(5-chloro-3-methyl-2- (piperidin-3- yloxy)phenyl)pyrrolo[2,1- f][1,2,4]triazine-6-carboxylic acid hydrochloride |
| | A single enantiomer | |
| 17. | 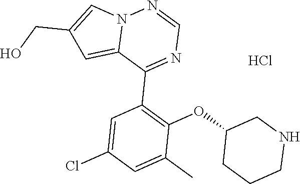 [SMILES: Cc1cc(Cl)cc(-c2ncnn3cc(CO)cc23)c1O[C@H]1CCCNC1 ; Cl] | (S)-(4-(5-chloro-3-methyl-2- (piperidin-3- yloxy)phenyl)pyrrolo[2,1- f][1,2,4]triazin-6-yl)methanol hydrochloride |
| | A single enantiomer | |
| 18. | 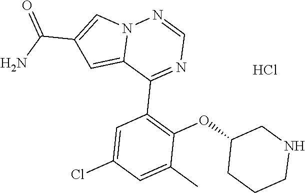 [SMILES: Cc1cc(Cl)cc(-c2ncnn3cc(C(N)=O)cc23)c1O[C@H]1CCCNC1 ; Cl] | (S)-4-(5-chloro-3-methyl-2- (piperidin-3- yloxy)phenyl)pyrrolo[2,1- f][1,2,4]triazine-6-carboxamide hydrochloride |
| | A single enantiomer | |
| 19. | 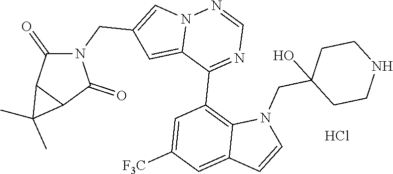 [SMILES: CC1(C)C2C(=O)N(Cc3cc4c(-c5cc(C(F)(F)F)cc6ccn(CC7(O)CCNCC7)c56)ncnn4c3)C(=O)C21 ; Cl] | 3-((4-(1-((4-hydroxypiperidin-4- yl)methyl)-5-(trifluoromethyl)-1H- indol-7-yl)pyrrolo[2,1- f][1,2,4]triazin-6-yl)methyl)-6,6- dimethyl-3-azabicyclo[3.1.0]hexane- 2,4-dione hydrochloride |
| 20. | 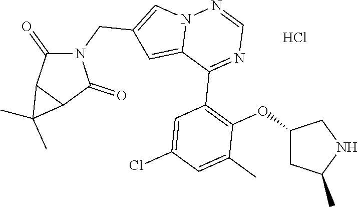 [SMILES: Cc1cc(Cl)cc(-c2ncnn3cc(CN4C(=O)C5C(C4=O)C5(C)C)cc23)c1O[C@@H]1CN[C@@H](C)C1 ; Cl] | 3-((4-(5-chloro-3-methyl-2- (((3S,5S)-5-methylpyrrolidin-3- yl)oxy)phenyl)pyrrolo[2,1- f][1,2,4]triazin-6-yl)methyl)-6,6- dimethyl-3-azabicyclo[3.1.0]hexane- 2,4-dione hydrochloride |
| | A single diastereoisomer | |
| 21. | 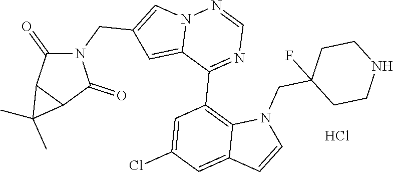 [SMILES: CC1(C)C2C(=O)N(Cc3cc4c(-c5cc(Cl)cc6ccn(CC7(F)CCNCC7)c56)ncnn4c3)C(=O)C21 ; Cl] | 3-((4-(5-chloro-1-((4- fluoropiperidin-4-yl)methyl)-1H- indol-7-yl)-5-methylpyrrolo[2,1- f][1,2,4]triazin-6-yl)methyl)-6,6- dimethyl-3-azabicyclo[3.1.0]hexane- 2,4-dione hydrochloride |
| | A mixture of atropoisomers | |
| 22. | 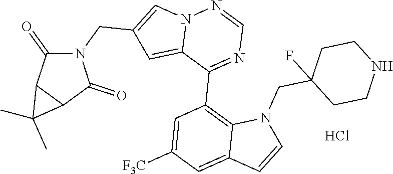 [SMILES: CC1(C)C2C(=O)N(Cc3cc4c(-c5cc(C(F)(F)F)cc6ccn(CC7(F)CCNCC7)c56)ncnn4c3)C(=O)C21 ; Cl] | 3-((4-(1-((4-fluoropiperidin-4- yl)methyl)-5-(trifluoromethyl)-1H- indol-7-yl)pyrrolo[2,1- f][1,2,4]triazin-6-yl)methyl)-6,6- dimethyl-3-azabicyclo[3.1.0]hexane- 2,4-dione hydrochloride |
| 23. | 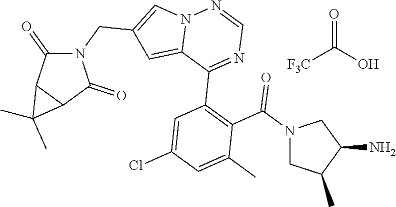 | 3-((4-(2-((3S,4S)-3-amino-4- methylpyrrolidine-1-carbonyl)-5- chloro-3-methylphenyl)pyrrolo[2,1- f][1,2,4]triazin-6-yl)methyl)-6,6- dimethyl-3-azabicyclo[3.1.0]hexane- 2,4-dione 2,2,2-trifluoroacetate |
| | A single diastereoisomer | |
| 24. | 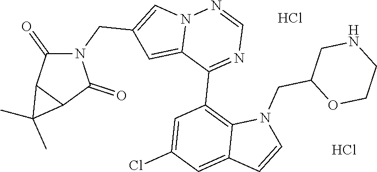 [SMILES: CC1(C)C2C(=O)N(Cc3cc4c(-c5cc(Cl)cc6ccn(CC7CNCCO7)c56)ncnn4c3)C(=O)C21 ; Cl ; Cl] | 3-((4-(5-chloro-1-(morpholin-2- ylmethyl)-1H-indol-7-yl)pyrrolo[2,1- f][1,2,4]triazin-6-yl)methyl)-6,6- dimethyl-3-azabicyclo[3.1.0]hexane- 2,4-dione dihydrochloride |
| | A racemate | |
| 25. | 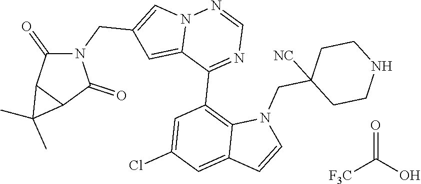 | 4-((5-chloro-7-(6-((6,6-dimethyl-2,4- dioxo-3-azabicyclo[3.1.0]hexan-3- yl)methyl)pyrrolo[2,1- f][1,2,4]triazin-4-yl)-1H-indol-1- yl)methyl)piperidine-4-carbonitrile 2,2,2-trifluoroacetate |
| 26. | 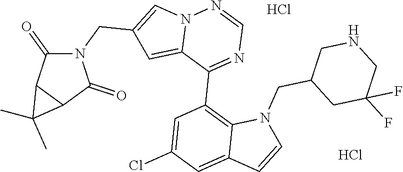 [SMILES: CC1(C)C2C(=O)N(Cc3cc4c(-c5cc(Cl)cc6ccn(CC7CNCC(F)(F)C7)c56)ncnn4c3)C(=O)C21 ; Cl ; Cl] | 3-((4-(5-chloro-1-((5,5- difluoropiperidin-3-yl)methyl)-1H- indol-7-yl)pyrrolo[2,1- f][1,2,4]triazin-6-yl)methyl)-6,6- dimethyl-3-azabicyclo[3.1.0]hexane- 2,4-dione dihydrochloride |
| | A racemate | |
| 27. | 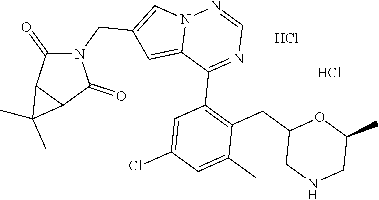 | 3-((4-(5-chloro-3-methyl-2-(((6S)-6- methylmorpholin-2- yl)methyl)phenyl)pyrrolo[2,1- f][1,2,4]triazin-6-yl)methyl)-6,6- dimethyl-3-azabicyclo[3.1.0]hexane- 2,4-dione dihydrochloride |
| | A single diastereoisomer | |
| 28. | 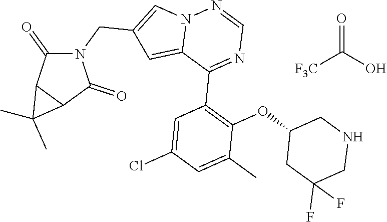 [SMILES: Cc1cc(Cl)cc(-c2ncnn3cc(CN4C(=O)C5C(C4=O)C5(C)C)cc23)c1O[C@@H]1CNCC(F)(F)C1 ; O=C(O)C(F)(F)F] | 3-((4-(5-chloro-2-(((S)-5,5- difluoropiperidin-3-yl)oxy)-3- methylphenyl)pyrrolo[2,1- f][1,2,4]triazin-6-yl)methyl)-6,6- dimethyl-3-azabicyclo[3.1.0]hexane- 2,4-dione 2,2,2-trifluoroacetate |
| | A single enantiomer | |
| 29. | 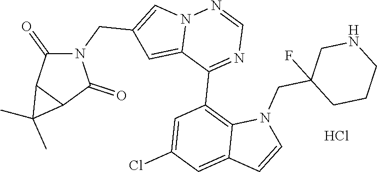 [SMILES: CC1(C)C2C(=O)N(Cc3cc4c(-c5cc(Cl)cc6ccn(CC7(F)CCCNC7)c56)ncnn4c3)C(=O)C21 ; Cl] | 3-((4-(5-chloro-1-((3- fluoropiperidin-3-yl)methyl)-1H- indol-7-yl)pyrrolo[2,1- f][1,2,4]triazin-6-yl)methyl)-6,6- dimethyl-3-azabicyclo[3.1.0]hexane- 2,4-dione hydrochloride |
| | A racemate | |
| 30. | 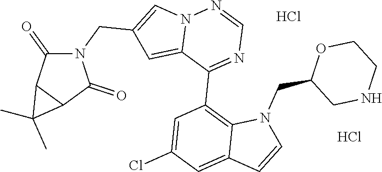 [SMILES: CC1(C)C2C(=O)N(Cc3cc4c(-c5cc(Cl)cc6ccn(C[C@@H]7CNCCO7)c56)ncnn4c3)C(=O)C21 ; Cl ; Cl] | 3-((4-(5-chloro-1-(((S)-morpholin-2- yl)methyl)-1H-indol-7- yl)pyrrolo[2,1-f][1,2,4]triazin-6- yl)methyl)-6,6-dimethyl-3- azabicyclo[3.1.0]hexane-2,4-dione dihydrochloride |
| | A single enantiomer of the compound 24 | |
| 31. | 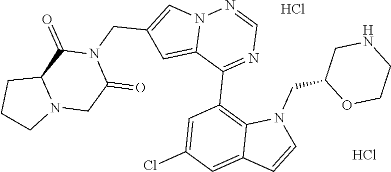 [SMILES: O=C1CN2CCC[C@H]2C(=O)N1Cc1cc2c(-c3cc(Cl)cc4ccn(C[C@@H]5CNCCO5)c34)ncnn2c1 ; Cl ; Cl] | (S)-2-((4-(5-chloro-1-(((S)- morpholin-2-yl)methyl)-1H-indol-7- yl)pyrrolo[2,1-f][1,2,4]triazin-6- yl)methyl)tetrahydropyrrolo[1,2- a]pyrazine-1,3(2H,4H)-dione dihydrochloride |
| | A single diastereoisomer | |
| 32. | 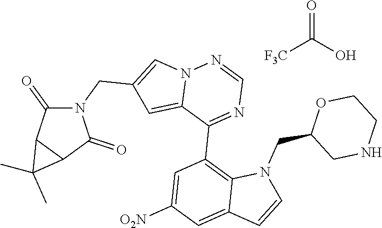 | 6,6-dimethyl-3-((4-(1-(((S)- morpholin-2-yl)methyl)-5-nitro-1H- indol-7-yl)pyrrolo[2,1- f][1,2,4]triazin-6-yl)methyl)-3- azabicyclo[3.1.0]hexane-2,4-dione 2,2,2-trifluoroacetate |
| | A single enantiomer | |
| 33. | 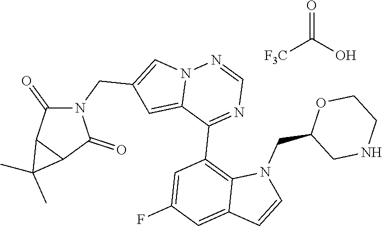 | 3-((4-(5-fluoro-1-(((S)-morpholin-2- yl)methyl)-1H-indol-7- yl)pyrrolo[2,1-f][1,2,4]triazin-6- yl)methyl)-6,6-dimethyl-3- azabicyclo[3.1.0]hexane-2,4-dione 2,2,2-trifluoroacetate |
| | A single enantiomer | |
| 34. | 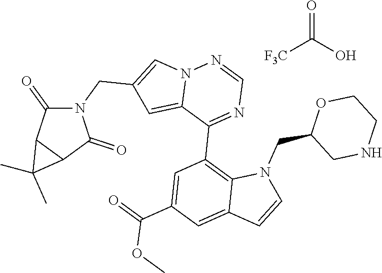 | methyl 7-(6-((6,6-dimethyl-2,4- dioxo-3-azabicyclo[3.1.0]hexan-3- yl)methyl)pyrrolo[2,1- f][1,2,4]triazin-4-yl)-1-(((S)- morpholin-2-yl)methyl)-1H-indole- 5-carboxylate 2,2,2-trifluoroacetate |
| | A single enantiomer | |
| 35. | 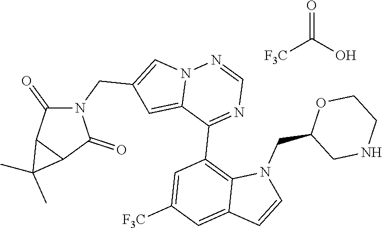 | 6,6-dimethyl-3-((4-(1-(((S)- morpholin-2-yl)methyl)-5- (trifluoromethyl)-1H-indol-7- yl)pyrrolo[2,1-f][1,2,4]triazin-6- yl)methyl)-3- azabicyclo[3.1.0]hexane-2,4-dione 2,2,2-trifluoroacetate |
| | A single enantiomer | |
| 36. | 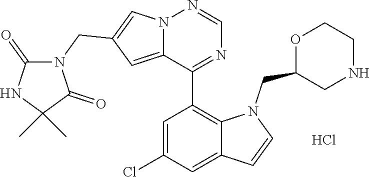 [SMILES: CC1(C)NC(=O)N(Cc2cc3c(-c4cc(Cl)cc5ccn(C[C@@H]6CNCCO6)c45)ncnn3c2)C1=O ; Cl] | (S)-3-((4-(5-chloro-1-(morpholin-2- ylmethyl)-1H-indol-7-yl)pyrrolo[2,1- f][1,2,4]triazin-6-yl)methyl)-5,5- dimethylimidazolidine-2,4-dione hydrochloride |
| | A single enantiomer | |
| 37. | 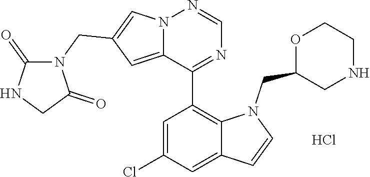 [SMILES: O=C1CNC(=O)N1Cc1cc2c(-c3cc(Cl)cc4ccn(C[C@@H]5CNCCO5)c34)ncnn2c1 ; Cl] | (S)-3-((4-(5-chloro-1-(morpholin-2- ylmethyl)-1H-indol-7-yl)pyrrolo[2,1- f][1,2,4]triazin-6- yl)methyl)imidazolidine-2,4-dione hydrochloride |
| | A single enantiomer | |
| 38. | 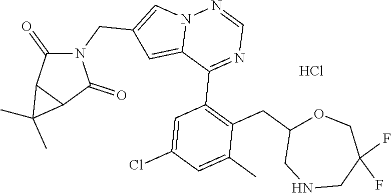 [SMILES: Cc1cc(Cl)cc(-c2ncnn3cc(CN4C(=O)C5C(C4=O)C5(C)C)cc23)c1CC1CNCC(F)(F)CO1 ; Cl] | 3-((4-(5-chloro-2-((6,6-difluoro-1,4- oxazepan-2-yl)methyl)-3- methylphenyl)pyrrolo[2,1- f][1,2,4]triazin-6-yl)methyl)-6,6- dimethyl-3-azabicyclo[3.1.0]hexane- 2,4-dione hydrochloride |
| | A racemate | |
| 39. | 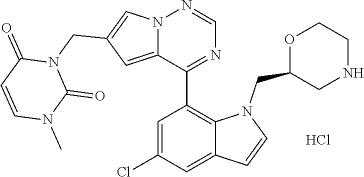 [SMILES: Cn1ccc(=O)n(Cc2cc3c(-c4cc(Cl)cc5ccn(C[C@@H]6CNCCO6)c45)ncnn3c2)c1=O ; Cl] | (S)-3-((4-(5-chloro-1-(morpholin-2- ylmethyl)-1H-indol-7-yl)pyrrolo[2,1- f][1,2,4]triazin-6-yl)methyl)-1- methylpyrimidine-2,4(1H,3H)-dione hydrochloride |
| | A single enantiomer | |
| 40. | 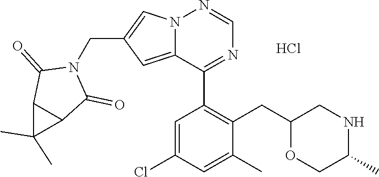 | 3-((4-(5-chloro-3-methyl-2-(((5R)-5- methylmorpholin-2- yl)methyl)phenyl)pyrrolo[2,1- f][1,2,4]triazin-6-yl)methyl)-6,6- dimethyl-3-azabicyclo[3.1.0]hexane- 2,4-dione hydrochloride |
| | A single diastereoisomer | |
| 41. | 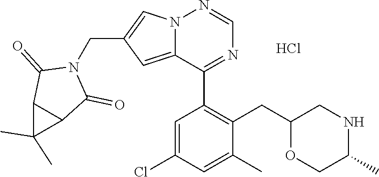 | 3-((4-(5-chloro-3-methyl-2-(((5R)-5- methylmorpholin-2- yl)methyl)phenyl)pyrrolo[2,1- f][1,2,4]triazin-6-yl)methyl)-6,6- dimethyl-3-azabicyclo[3.1.0]hexane- 2,4-dione hydrochloride |
| | A single diastereoisomer | |
| 42. | 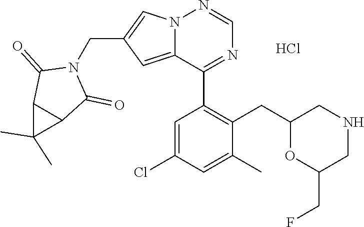 [SMILES: Cc1cc(Cl)cc(-c2ncnn3cc(CN4C(=O)C5C(C4=O)C5(C)C)cc23)c1CC1CNCC(CF)O1 ; Cl] | 3-((4-(5-chloro-2-((6- (fluoromethyl)morpholin-2- yl)methyl)-3- methylphenyl)pyrrolo[2,1- f][1,2,4]triazin-6-yl)methyl)-6,6- dimethyl-3-azabicyclo[3.1.0]hexane- 2,4-dione hydrochloride |
| | A single isomer | |
| 43. | 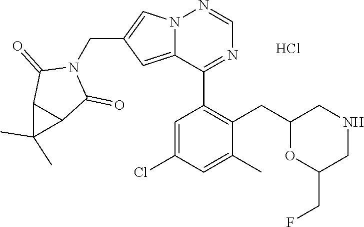 [SMILES: Cc1cc(Cl)cc(-c2ncnn3cc(CN4C(=O)C5C(C4=O)C5(C)C)cc23)c1CC1CNCC(CF)O1 ; Cl] | 3-((4-(5-chloro-2-((6- (fluoromethyl)morpholin-2- yl)methyl)-3- methylphenyl)pyrrolo[2,1- f][1,2,4]triazin-6-yl)methyl)-6,6- dimethyl-3-azabicyclo[3.1.0]hexane- 2,4-dione hydrochloride |
| | A single isomer | |
| 44. | 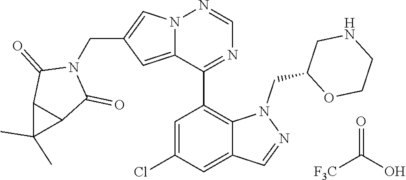 | 3-((4-(5-chloro-1-(((S)-morpholin-2- yl)methyl)-1H-indazol-7- yl)pyrrolo[2,1-f][1,2,4]triazin-6- yl)methyl)-6,6-dimethyl-3- azabicyclo[3.1.0]hexane-2,4-dione 2,2,2-trifluoroacetate |
| | A single enantiomer | |
| 45. | 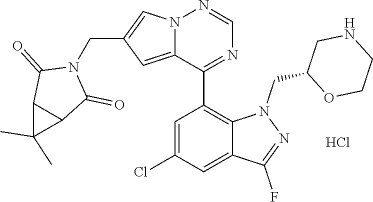 [SMILES: CC1(C)C2C(=O)N(Cc3cc4c(-c5cc(Cl)cc6c(F)nn(C[C@@H]7CNCCO7)c56)ncnn4c3)C(=O)C21 ; Cl] | 3-((4-(5-chloro-3-fluoro-1-(((S)- morpholin-2-yl)methyl)-1H-indazol- 7-yl)pyrrolo[2,1-f][1,2,4]triazin-6- yl)methyl)-6,6-dimethyl-3- azabicyclo[3.1.0]hexane-2,4-dione hydrochloride |
| | A single enantiomer | |
| 46. | 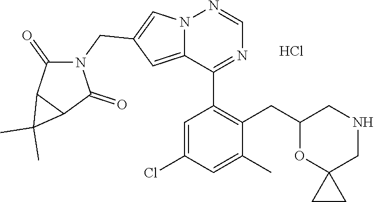 [SMILES: Cc1cc(Cl)cc(-c2ncnn3cc(CN4C(=O)C5C(C4=O)C5(C)C)cc23)c1CC1CNCC2(CC2)O1 ; Cl] | 3-((4-(2-((4-oxa-7- azaspiro[2.5]octan-5-yl)methyl)-5- chloro-3-methylphenyl)pyrrolo[2,1- f][1,2,4]triazin-6-yl)methyl)-6,6- dimethyl-3-azabicyclo[3.1.0]hexane- 2,4-dione hydrochloride |
| | A racemate | |
| 47. | 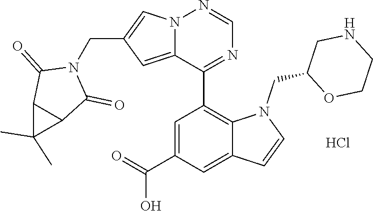 [SMILES: CC1(C)C2C(=O)N(Cc3cc4c(-c5cc(C(=O)O)cc6ccn(C[C@@H]7CNCCO7)c56)ncnn4c3)C(=O)C21 ; Cl] | 7-(6-((6,6-dimethyl-2,4-dioxo-3- azabicyclo[3.1.0]hexan-3- yl)methyl)pyrrolo[2,1- f][1,2,4]triazin-4-yl)-1-(((S)- morpholin-2-yl)methyl)-1H-indole- 5-carboxylic acid hydrochloride |
| | A single enantiomer | |
| 48. | 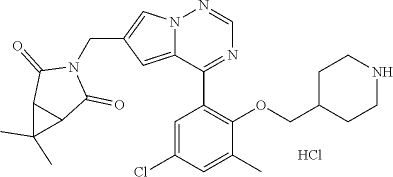 [SMILES: Cc1cc(Cl)cc(-c2ncnn3cc(CN4C(=O)C5C(C4=O)C5(C)C)cc23)c1OCC1CCNCC1 ; Cl] | 3-((4-(5-chloro-3-methyl-2- (piperidin-4- ylmethoxy)phenyl)pyrrolo[2,1- f][1,2,4]triazin-6-yl)methyl)-6,6- dimethyl-3-azabicyclo[3.1.0]hexane- 2,4-dione hydrochloride |
| 49. | 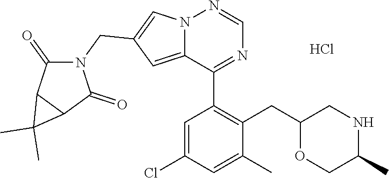 | 3-((4-(5-chloro-3-methyl-2-(((5S)-5- methylmorpholin-2- yl)methyl)phenyl)pyrrolo[2,1- f][1,2,4]triazin-6-yl)methyl)-6,6- dimethyl-3-azabicyclo[3.1.0]hexane- 2,4-dione hydrochloride |
| | A single diastereoisomer | |
| 50. | 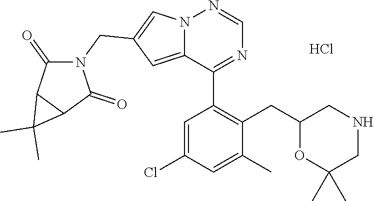 [SMILES: Cc1cc(Cl)cc(-c2ncnn3cc(CN4C(=O)C5C(C4=O)C5(C)C)cc23)c1CC1CNCC(C)(C)O1 ; Cl] | 3-((4-(5-chloro-2-((6,6- dimethylmorpholin-2-yl)methyl)-3- methylphenyl)pyrrolo[2,1- f][1,2,4]triazin-6-yl)methyl)-6,6- dimethyl-3-azabicyclo[3.1.0]hexane- 2,4-dione hydrochloride |
| | A racemate | |
| 51. | 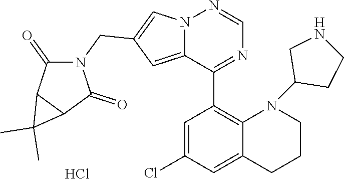 [SMILES: CC1(C)C2C(=O)N(Cc3cc4c(-c5cc(Cl)cc6c5N(C5CCNC5)CCC6)ncnn4c3)C(=O)C21 ; Cl] | 3-((4-(6-chloro-1-(pyrrolidin-3-yl)- 1,2,3,4-tetrahydroquinolin-8- yl)pyrrolo[2,1-f][1,2,4]triazin-6- yl)methyl)-6,6-dimethyl-3- azabicyclo[3.1.0]hexane-2,4-dione hydrochloride |
| | A racemate | |
| 52. | 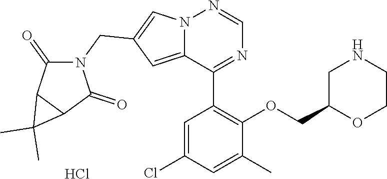 [SMILES: Cc1cc(Cl)cc(-c2ncnn3cc(CN4C(=O)C5C(C4=O)C5(C)C)cc23)c1OC[C@H]1CNCCO1 ; Cl] | 3-((4-(5-chloro-3-methyl-2-(((R)- morpholin-2- yl)methoxy)phenyl)pyrrolo[2,1- f][1,2,4]triazin-6-yl)methyl)-6,6- dimethyl-3-azabicyclo[3.1.0]hexane- 2,4-dione hydrochloride |
| | A single enantiomer | |
| 53. | 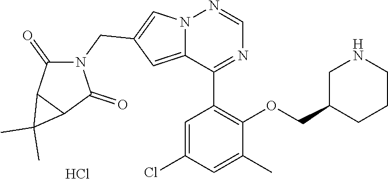 [SMILES: Cc1cc(Cl)cc(-c2ncnn3cc(CN4C(=O)C5C(C4=O)C5(C)C)cc23)c1OC[C@@H]1CCCNC1 ; Cl] | 3-((4-(5-chloro-3-methyl-2-(((R)- piperidin-3- yl)methoxy)phenyl)pyrrolo[2,1- f][1,2,4]triazin-6-yl)methyl)-6,6- dimethyl-3-azabicyclo[3.1.0]hexane- 2,4-dione hydrochloride |
| | A single enantiomer | |
| 54. | 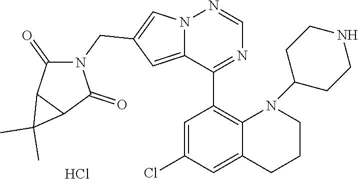 [SMILES: CC1(C)C2C(=O)N(Cc3cc4c(-c5cc(Cl)cc6c5N(C5CCNCC5)CCC6)ncnn4c3)C(=O)C21 ; Cl] | 3-((4-(6-chloro-1-(piperidin-4-yl)- 1,2,3,4-tetrahydroquinolin-8- yl)pyrrolo[2,1-f][1,2,4]triazin-6- yl)methyl)-6,6-dimethyl-3- azabicyclo[3.1.0]hexane-2,4-dione hydrochloride |
| 55. | 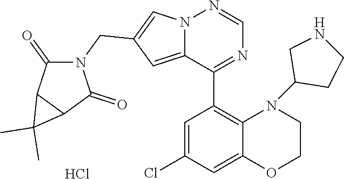 [SMILES: CC1(C)C2C(=O)N(Cc3cc4c(-c5cc(Cl)cc6c5N(C5CCNC5)CCO6)ncnn4c3)C(=O)C21 ; Cl] | 3-((4-(7-chloro-4-(pyrrolidin-3-yl)- 3,4-dihydro-2H- benzo[b][1,4]oxazin-5- yl)pyrrolo[2,1-f][1,2,4]triazin-6- yl)methyl)-6,6-dimethyl-3- azabicyclo[3.1.0]hexane-2,4-dione hydrochloride |
| | A racemate | |
| 56. | 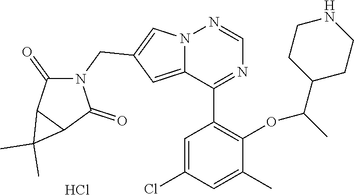 [SMILES: Cc1cc(Cl)cc(-c2ncnn3cc(CN4C(=O)C5C(C4=O)C5(C)C)cc23)c1OC(C)C1CCNCC1 ; Cl] | 3-((4-(5-chloro-3-methyl-2-(1- (piperidin-4- yl)ethoxy)phenyl)pyrrolo[2,1- f][1,2,4]triazin-6-yl)methyl)-6,6- dimethyl-3-azabicyclo[3.1.0]hexane- 2,4-dione hydrochloride |
| | A single enantiomer | |
| 57. | 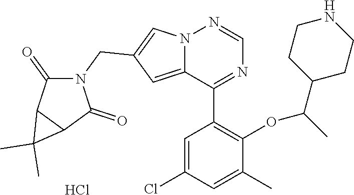 [SMILES: Cc1cc(Cl)cc(-c2ncnn3cc(CN4C(=O)C5C(C4=O)C5(C)C)cc23)c1OC(C)C1CCNCC1 ; Cl] | 3-((4-(5-chloro-3-methyl-2-(1- (piperidin-4- yl)ethoxy)phenyl)pyrrolo[2,1- f][1,2,4]triazin-6-yl)methyl)-6,6- dimethyl-3-azabicyclo[3.1.0]hexane- 2,4-dione hydrochloride |
| | A single enantiomer | |
| 58. | 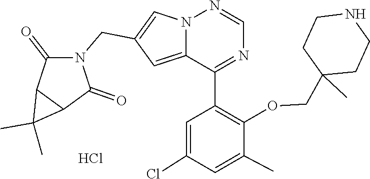 [SMILES: Cc1cc(Cl)cc(-c2ncnn3cc(CN4C(=O)C5C(C4=O)C5(C)C)cc23)c1OCC1(C)CCNCC1 ; Cl] | 3-((4-(5-chloro-3-methyl-2-((4- methylpiperidin-4- yl)methoxy)phenyl)pyrrolo[2,1- f][1,2,4]triazin-6-yl)methyl)-6,6- dimethyl-3-azabicyclo[3.1.0]hexane- 2,4-dione hydrochloride |
| 59. | 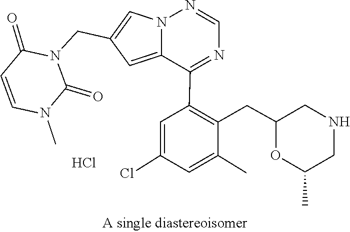 [SMILES: Cc1cc(Cl)cc(-c2ncnn3cc(Cn4c(=O)ccn(C)c4=O)cc23)c1CC1CNC[C@H](C)O1 ; Cl] | 3-((4-(5-chloro-3-methyl-2-(((6S)-6- methylmorpholin-2- yl)methyl)phenyl)pyrrolo[2,1- f][1,2,4]triazin-6-yl)methyl)-1- methylpyrimidine-2,4(1H,3H)-dione hydrochloride |
| 60. | 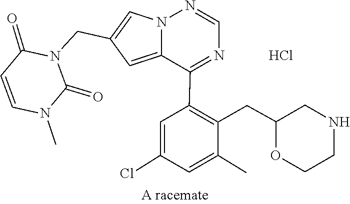 [SMILES: Cc1cc(Cl)cc(-c2ncnn3cc(Cn4c(=O)ccn(C)c4=O)cc23)c1CC1CNCCO1 ; Cl] | 3-((4-(5-chloro-3-methyl-2- (morpholin-2- ylmethyl)phenyl)pyrrolo[2,1- f][1,2,4]triazin-6-yl)methyl)-1- methylpyrimidine-2,4(1H,3H)-dione hydrochloride |
| 61. | 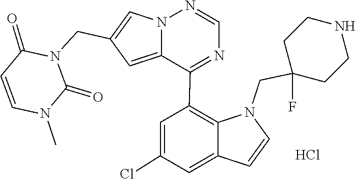 [SMILES: Cn1ccc(=O)n(Cc2cc3c(-c4cc(Cl)cc5ccn(CC6(F)CCNCC6)c45)ncnn3c2)c1=O ; Cl] | 3-((4-(5-chloro-1-((4- fluoropiperidin-4-yl)methyl)-1H- indol-7-yl)pyrrolo[2,1- f][1,2,4]triazin-6-yl)methyl)-1- methylpyrimidine-2,4(1H,3H)-dione hydrochloride |
| 62. | 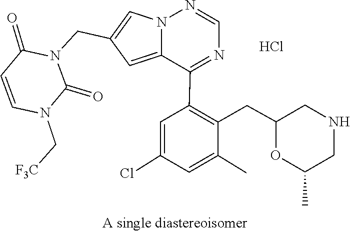 [SMILES: Cc1cc(Cl)cc(-c2ncnn3cc(Cn4c(=O)ccn(CC(F)(F)F)c4=O)cc23)c1CC1CNC[C@H](C)O1 ; Cl] | 3-((4-(5-chloro-3-methyl-2-(((6S)-6- methylmorpholin-2- yl)methyl)phenyl)pyrrolo[2,1- f][1,2,4]triazin-6-yl)methyl)-1- (2,2,2-trifluoroethyl)pyrimidine- 2,4(1H,3H)-dione hydrochloride |
| 63. | 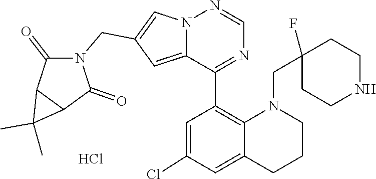 [SMILES: CC1(C)C2C(=O)N(Cc3cc4c(-c5cc(Cl)cc6c5N(CC5(F)CCNCC5)CCC6)ncnn4c3)C(=O)C21 ; Cl] | 3-((4-(6-chloro-1-((4- fluoropiperidin-4-yl)methyl)-1,2,3,4- tetrahydroquinolin-8-yl)pyrrolo[2,1- f][1,2,4]triazin-6-yl)methyl)-6,6- dimethyl-3-azabicyclo[3.1.0]hexane- 2,4-dione hydrochloride |
| 64. | 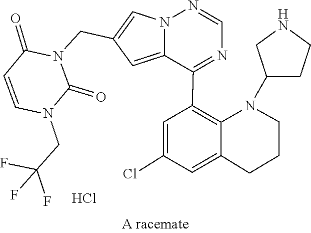 [SMILES: O=c1ccn(CC(F)(F)F)c(=O)n1Cc1cc2c(-c3cc(Cl)cc4c3N(C3CCNC3)CCC4)ncnn2c1 ; Cl] | 3-((4-(6-chloro-1-(pyrrolidin-3-yl)- 1,2,3,4-tetrahydroquinolin-8- yl)pyrrolo[2,1-f][1,2,4]triazin-6- yl)methyl)-1-(2,2,2- trifluoroethyl)pyrimidine- 2,4(1H,3H)-dione hydrochloride |
| 65. | 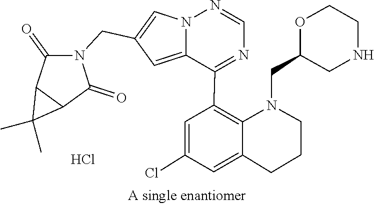 [SMILES: CC1(C)C2C(=O)N(Cc3cc4c(-c5cc(Cl)cc6c5N(C[C@@H]5CNCCO5)CCC6)ncnn4c3)C(=O)C21 ; Cl] | 3-((4-(6-chloro-1-(((S)-morpholin-2- yl)methyl)-1,2,3,4- tetrahydroquinolin-8-yl)pyrrolo[2,1- f][1,2,4]triazin-6-yl)methyl)-6,6- dimethyl-3-azabicyclo[3.1.0]hexane- 2,4-dione hydrochloride |
| 66. | 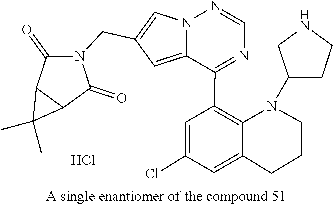 [SMILES: CC1(C)C2C(=O)N(Cc3cc4c(-c5cc(Cl)cc6c5N(C5CCNC5)CCC6)ncnn4c3)C(=O)C21 ; Cl] | 3-((4-(6-chloro-1-(pyrrolidin-3-yl)- 1,2,3,4-tetrahydroquinolin-8- yl)pyrrolo[2,1-f][1,2,4]triazin-6- yl)methyl)-6,6-dimethyl-3- azabicyclo[3.1.0]hexane-2,4-dione hydrochloride |
| 67. | 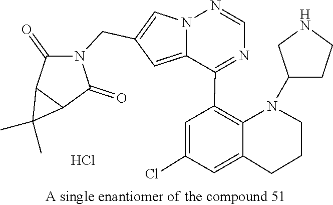 [SMILES: CC1(C)C2C(=O)N(Cc3cc4c(-c5cc(Cl)cc6c5N(C5CCNC5)CCC6)ncnn4c3)C(=O)C21 ; Cl] | 3-((4-(6-chloro-1-(pyrrolidin-3-yl)- 1,2,3,4-tetrahydroquinolin-8- yl)pyrrolo[2,1-f][1,2,4]triazin-6- yl)methyl)-6,6-dimethyl-3- azabicyclo[3.1.0]hexane-2,4-dione hydrochloride |
| 68. | 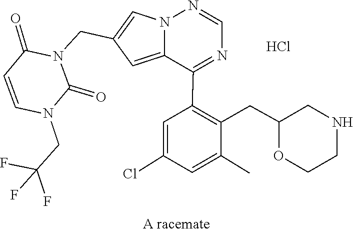 [SMILES: Cc1cc(Cl)cc(-c2ncnn3cc(Cn4c(=O)ccn(CC(F)(F)F)c4=O)cc23)c1CC1CNCCO1 ; Cl] | 3-((4-(5-chloro-3-methyl-2- (morpholin-2- ylmethyl)phenyl)pyrrolo[2,1- f][1,2,4]triazin-6-yl)methyl)-1- (2,2,2-trifluoroethyl)pyrimidine- 2,4(1H,3H)-dione hydrochloride |
| 69. | 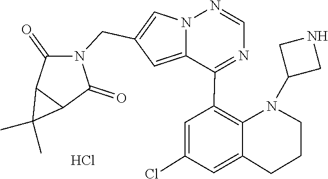 [SMILES: CC1(C)C2C(=O)N(Cc3cc4c(-c5cc(Cl)cc6c5N(C5CNC5)CCC6)ncnn4c3)C(=O)C21 ; Cl] | 3-((4-(1-(azetidin-3-yl)-6-chloro- 1,2,3,4-tetrahydroquinolin-8- yl)pyrrolo[2,1-f][1,2,4]triazin-6- yl)methyl)-6,6-dimethyl-3- azabicyclo[3.1.0]hexane-2,4-dione hydrochloride |
| 70. | 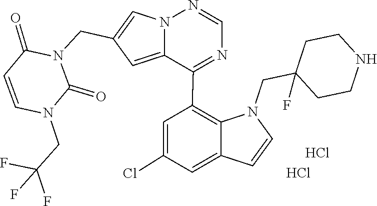 [SMILES: O=c1ccn(CC(F)(F)F)c(=O)n1Cc1cc2c(-c3cc(Cl)cc4ccn(CC5(F)CCNCC5)c34)ncnn2c1 ; Cl ; Cl] | 3-((4-(5-chloro-1-((4- fluoropiperidin-4-yl)methyl)-1H- indol-7-yl)pyrrolo[2,1- f][1,2,4]triazin-6-yl)methyl)-1- (2,2,2-trifluoroethyl)pyrimidine- 2,4(1H,3H)-dione dihydrochloride |
| 71. | 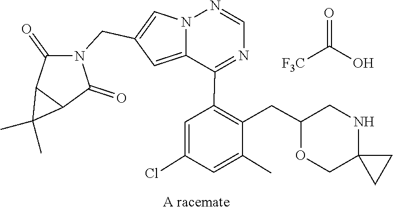 [SMILES: Cc1cc(Cl)cc(-c2ncnn3cc(CN4C(=O)C5C(C4=O)C5(C)C)cc23)c1CC1CNC2(CC2)CO1 ; O=C(O)C(F)(F)F] | 3-((4-(2-((7-oxa-4- azaspiro[2.5]octan-6-yl)methyl)-5- chloro-3-methylphenyl)pyrrolo[2,1- f][1,2,4]triazin-6-yl)methyl)-6,6- dimethyl-3-azabicyclo[3.1.0]hexane- 2,4-dione 2,2,2-trifluoroacetate |
| 72. | 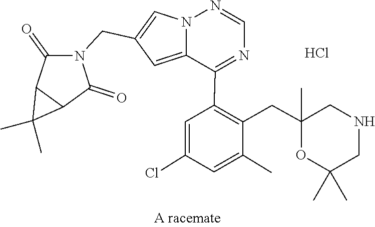 [SMILES: Cc1cc(Cl)cc(-c2ncnn3cc(CN4C(=O)C5C(C4=O)C5(C)C)cc23)c1CC1(C)CNCC(C)(C)O1 ; Cl] | 3-((4-(5-chloro-3-methyl-2-((2,6,6- trimethylmorpholin-2- yl)methyl)phenyl)pyrrolo[2,1- f][1,2,4]triazin-6-yl)methyl)-6,6- dimethyl-3-azabicyclo[3.1.0]hexane- 2,4-dione hydrochloride |
| 73. | 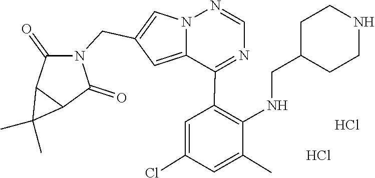 [SMILES: Cc1cc(Cl)cc(-c2ncnn3cc(CN4C(=O)C5C(C4=O)C5(C)C)cc23)c1NCC1CCNCC1 ; Cl ; Cl] | 3-((4-(5-chloro-3-methyl-2- ((piperidin-4- ylmethyl)amino)phenyl)pyrrolo[2,1- f][1,2,4]triazin-6-yl)methyl)-6,6- dimethyl-3-azabicyclo[3.1.0]hexane- 2,4-dione dihydrochloride |
| 74. | 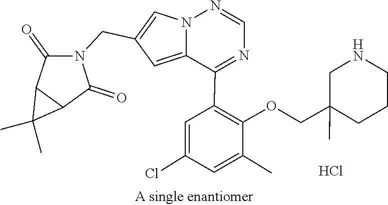 [SMILES: Cc1cc(Cl)cc(-c2ncnn3cc(CN4C(=O)C5C(C4=O)C5(C)C)cc23)c1OCC1(C)CCCNC1 ; Cl] | 3-((4-(5-chloro-3-methyl-2-((3- methylpiperidin-3- yl)methoxy)phenyl)pyrrolo[2,1- f][1,2,4]triazin-6-yl)methyl)-6,6- dimethyl-3-azabicyclo[3.1.0]hexane- 2,4-dione hydrochloride |
| 75. | 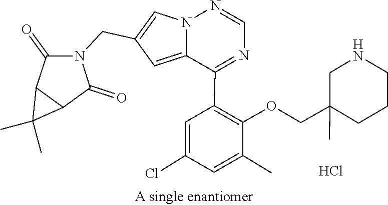 [SMILES: Cc1cc(Cl)cc(-c2ncnn3cc(CN4C(=O)C5C(C4=O)C5(C)C)cc23)c1OCC1(C)CCCNC1 ; Cl] | 3-((4-(5-chloro-3-methyl-2-((3- methylpiperidin-3- yl)methoxy)phenyl)pyrrolo[2,1- f][1,2,4]triazin-6-yl)methyl)-6,6- dimethyl-3-azabicyclo[3.1.0]hexane- 2,4-dione hydrochloride |
| 76. | 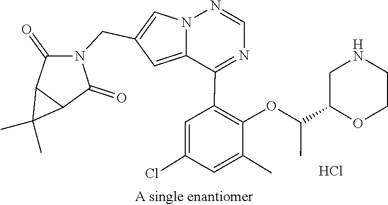 [SMILES: Cc1cc(Cl)cc(-c2ncnn3cc(CN4C(=O)C5C(C4=O)C5(C)C)cc23)c1OC(C)[C@@H]1CNCCO1 ; Cl] | 3-((4-(5-chloro-3-methyl-2-((R)-1- ((S)-morpholin-2- yl)ethoxy)phenyl)pyrrolo[2,1- f][1,2,4]triazin-6-yl)methyl)-6,6- dimethyl-3-azabicyclo[3.1.0]hexane- 2,4-dione hydrochloride |
| 77. | 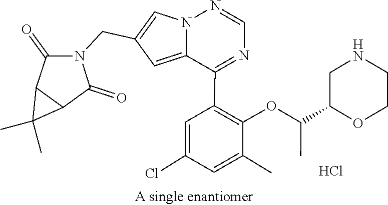 [SMILES: Cc1cc(Cl)cc(-c2ncnn3cc(CN4C(=O)C5C(C4=O)C5(C)C)cc23)c1OC(C)[C@@H]1CNCCO1 ; Cl] | 3-((4-(5-chloro-3-methyl-2-((R)-1- ((S)-morpholin-2- yl)ethoxy)phenyl)pyrrolo[2,1- f][1,2,4]triazin-6-yl)methyl)-6,6- dimethyl-3-azabicyclo[3.1.0]hexane- 2,4-dione hydrochloride |
| 78. | 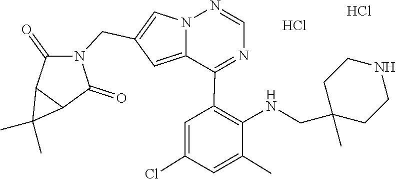 [SMILES: Cc1cc(Cl)cc(-c2ncnn3cc(CN4C(=O)C5C(C4=O)C5(C)C)cc23)c1NCC1(C)CCNCC1 ; Cl ; Cl] | 3-((4-(5-chloro-3-methyl-2-(((4- methylpiperidin-4- yl)methyl)amino)phenyl)pyrrolo[2,1- f][1,2,4]triazin-6-yl)methyl)-6,6- dimethyl-3-azabicyclo[3.1.0]hexane- 2,4-dione dihydrochloride |
| 79. | 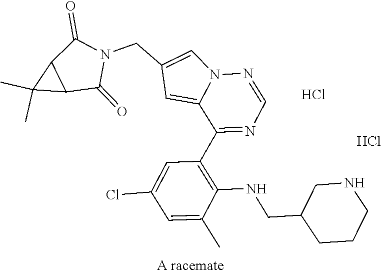 [SMILES: Cc1cc(Cl)cc(-c2ncnn3cc(CN4C(=O)C5C(C4=O)C5(C)C)cc23)c1NCC1CCCNC1 ; Cl ; Cl] | 3-((4-(5-chloro-3-methyl-2- ((piperidin-3- ylmethyl)amino)phenyl)pyrrolo[2,1- f][1,2,4]triazin-6-yl)methyl)-6,6- dimethyl-3-azabicyclo[3.1.0]hexane- 2,4-dione dihydrochloride |
| 80. | 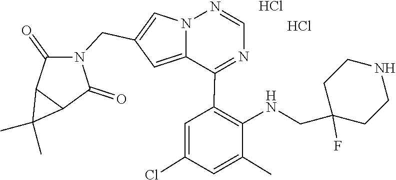 [SMILES: Cl ; Cc1cc(Cl)cc(-c2ncnn3cc(CN4C(=O)C5C(C4=O)C5(C)C)cc23)c1NCC1(F)CCNCC1 ; Cl] | 3-((4-(5-chloro-2-(((4- fluoropiperidin-4-yl)methyl)amino)- 3-methylphenyl)pyrrolo[2,1- f][1,2,4]triazin-6-yl)methyl)-6,6- dimethyl-3-azabicyclo[3.1.0]hexane- 2,4-dione dihydrochloride |
| 81. | 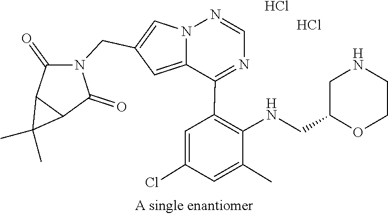 [SMILES: Cl ; Cc1cc(Cl)cc(-c2ncnn3cc(CN4C(=O)C5C(C4=O)C5(C)C)cc23)c1NC[C@@H]1CNCCO1 ; Cl] | 3-((4-(5-chloro-3-methyl-2-((((S)- morpholin-2- yl)methyl)amino)phenyl)pyrrolo[2,1- f][1,2,4]triazin-6-yl)methyl)-6,6- dimethyl-3-azabicyclo[3.1.0]hexane- 2,4-dione dihydrochloride |
| 82. | 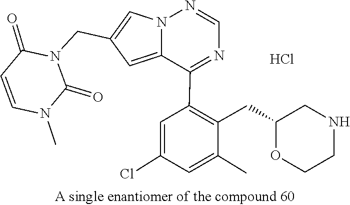 [SMILES: Cc1cc(Cl)cc(-c2ncnn3cc(Cn4c(=O)ccn(C)c4=O)cc23)c1C[C@@H]1CNCCO1 ; Cl] | (R)-3-((4-(5-chloro-3-methyl-2- (morpholin-2- ylmethyl)phenyl)pyrrolo[2,1- f][1,2,4]triazin-6-yl)methyl)-1- methylpyrimidine-2,4(1H,3H)-dione hydrochloride |
| 83. | 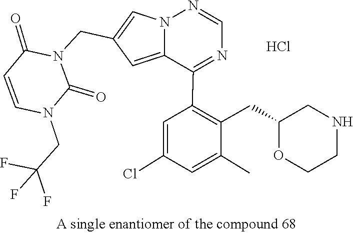 [SMILES: Cc1cc(Cl)cc(-c2ncnn3cc(Cn4c(=O)ccn(CC(F)(F)F)c4=O)cc23)c1C[C@@H]1CNCCO1 ; Cl] | (R)-3-((4-(5-chloro-3-methyl-2- (morpholin-2- ylmethyl)phenyl)pyrrolo[2,1- f][1,2,4]triazin-6-yl)methyl)-1- (2,2,2-trifluoroethyl)pyrimidine- 2,4(1H,3H)-dione hydrochloride |
| 84. | 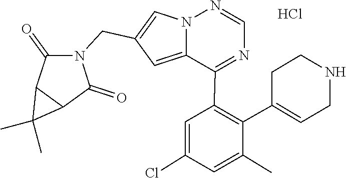 [SMILES: Cc1cc(Cl)cc(-c2ncnn3cc(CN4C(=O)C5C(C4=O)C5(C)C)cc23)c1C1=CCNCC1 ; Cl] | 3-((4-(5-chloro-3-methyl-2-(1,2,3,6- tetrahydropyridin-4- yl)phenyl)pyrrolo[2,1- f][1,2,4]triazin-6-yl)methyl)-6,6- dimethyl-3-azabicyclo[3.1.0]hexane- 2,4-dione hydrochloride |
| 85. | 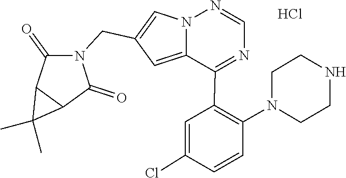 [SMILES: CC1(C)C2C(=O)N(Cc3cc4c(-c5cc(Cl)ccc5N5CCNCC5)ncnn4c3)C(=O)C21 ; Cl] | 3-((4-(5-chloro-2-(piperazin-1- yl)phenyl)pyrrolo[2,1- f][1,2,4]triazin-6-yl)methyl)-6,6- dimethyl-3-azabicyclo[3.1.0]hexane- 2,4-dione hydrochloride |
| 86. | 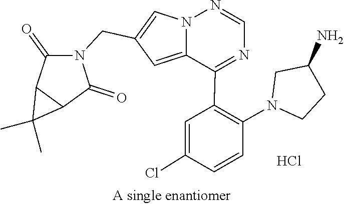 [SMILES: CC1(C)C2C(=O)N(Cc3cc4c(-c5cc(Cl)ccc5N5CC[C@H](N)C5)ncnn4c3)C(=O)C21 ; Cl] | 3-((4-(2-((S)-3-aminopyrrolidin-1- yl)-5-chlorophenyl)pyrrolo[2,1- f][1,2,4]triazin-6-yl)methyl)-6,6- dimethyl-3-azabicyclo[3.1.0]hexane- 2,4-dione hydrochloride |
| 87. | 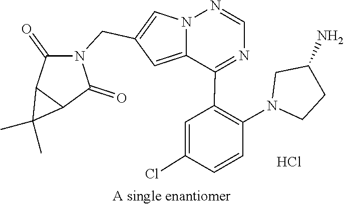 [SMILES: CC1(C)C2C(=O)N(Cc3cc4c(-c5cc(Cl)ccc5N5CC[C@@H](N)C5)ncnn4c3)C(=O)C21 ; Cl] | 3-((4-(2-((R)-3-aminopyrrolidin-1- yl)-5-chlorophenyl)pyrrolo[2,1- f][1,2,4]triazin-6-yl)methyl)-6,6- dimethyl-3-azabicyclo[3.1.0]hexane- 2,4-dione hydrochloride |
| 88. | 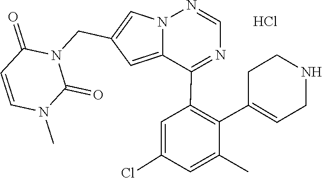 [SMILES: Cc1cc(Cl)cc(-c2ncnn3cc(Cn4c(=O)ccn(C)c4=O)cc23)c1C1=CCNCC1 ; Cl] | 3-((4-(5-chloro-3-methyl-2-(1,2,3,6- tetrahydropyridin-4- yl)phenyl)pyrrolo[2,1- f][1,2,4]triazin-6-yl)methyl)-1- methylpyrimidine-2,4(1H,3H)-dione hydrochloride |
| 89. | 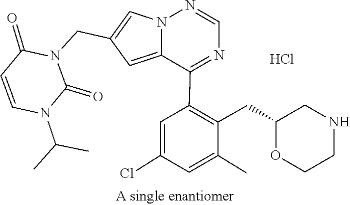 [SMILES: Cc1cc(Cl)cc(-c2ncnn3cc(Cn4c(=O)ccn(C(C)C)c4=O)cc23)c1C[C@@H]1CNCCO1 ; Cl] | (R)-3-((4-(5-chloro-3-methyl-2- (morpholin-2- ylmethyl)phenyl)pyrrolo[2,1- f][1,2,4]triazin-6-yl)methyl)-1- isopropylpyrimidine-2,4(1H,3H)- dione hydrochloride |
| 90. | 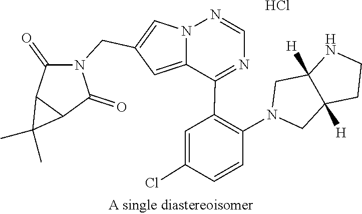 [SMILES: Cl ; CC1(C)C2C(=O)N(Cc3cc4c(-c5cc(Cl)ccc5N5C[C@H]6CCN[C@H]6C5)ncnn4c3)C(=O)C21] | 3-((4-(5-chloro-2-((3aR,6aR)- hexahydropyrrolo[3,4-b]pyrrol- 5(1H)-yl)phenyl)pyrrolo[2,1- f][1,2,4]triazin-6-yl)methyl)-6,6- dimethyl-3-azabicyclo[3.1.0]hexane- 2,4-dione hydrochloride |
| 91. | 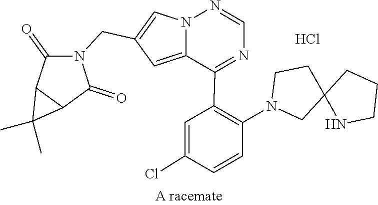 [SMILES: CC1(C)C2C(=O)N(Cc3cc4c(-c5cc(Cl)ccc5N5CCC6(CCCN6)C5)ncnn4c3)C(=O)C21 ; Cl] | 3-((4-(5-chloro-2-(1,7- diazaspiro[4.4]nonan-7- yl)phenyl)pyrrolo[2,1- f][1,2,4]triazin-6-yl)methyl)-6,6- dimethyl-3-azabicyclo[3.1.0]hexane- 2,4-dione hydrochloride |
| 92. | 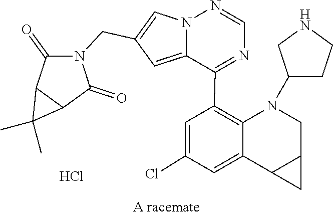 [SMILES: CC1(C)C2C(=O)N(Cc3cc4c(-c5cc(Cl)cc6c5N(C5CCNC5)CC5CC65)ncnn4c3)C(=O)C21 ; Cl] | 3-((4-(6-chloro-3-(pyrrolidin-3-yl)- 1a,2,3,7b-tetrahydro-1H- cyclopropa[c]quinolin-4- yl)pyrrolo[2,1-f][1,2,4]triazin-6- yl)methyl)-6,6-dimethyl-3- azabicyclo[3.1.0]hexane-2,4-dione hydrochloride |
| 93. | 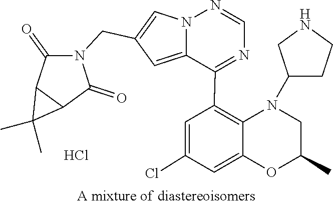 [SMILES: C[C@@H]1CN(C2CCNC2)c2c(cc(Cl)cc2-c2ncnn3cc(CN4C(=O)C5C(C4=O)C5(C)C)cc23)O1 ; Cl] | 3-((4-((2R)-7-chloro-2-methyl-4- (pyrrolidin-3-yl)-3,4-dihydro-2H- benzo[b][1,4]oxazin-5- yl)pyrrolo[2,1-f][1,2,4]triazin-6- yl)methyl)-6,6-dimethyl-3- azabicyclo[3.1.0]hexane-2,4-dione hydrochloride |
| 94. | 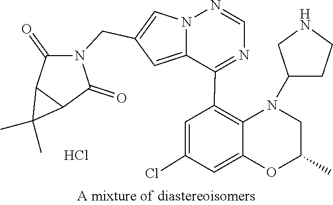 [SMILES: C[C@H]1CN(C2CCNC2)c2c(cc(Cl)cc2-c2ncnn3cc(CN4C(=O)C5C(C4=O)C5(C)C)cc23)O1 ; Cl] | 3-((4-((2S)-7-chloro-2-methyl-4- (pyrrolidin-3-yl)-3,4-dihydro-2H- benzo[b][1,4]oxazin-5- yl)pyrrolo[2,1-f][1,2,4]triazin-6- yl)methyl)-6,6-dimethyl-3- azabicyclo[3.1.0]hexane-2,4-dione hydrochloride |
| 95. | 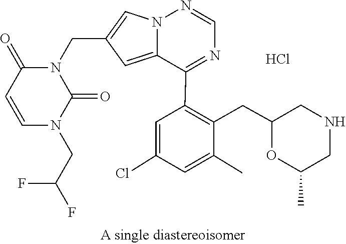 [SMILES: Cc1cc(Cl)cc(-c2ncnn3cc(Cn4c(=O)ccn(CC(F)F)c4=O)cc23)c1CC1CNC[C@H](C)O1 ; Cl] | 3-((4-(5-chloro-3-methyl-2-(((6S)-6- methylmorpholin-2- yl)methyl)phenyl)pyrrolo[2,1- f][1,2,4]triazin-6-yl)methyl)-1-(2,2- difluoroethyl)pyrimidine- 2,4(1H,3H)-dione hydrochloride |
| 96. | 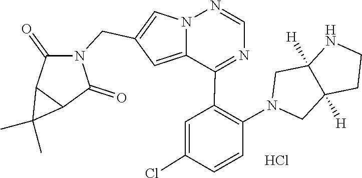 [SMILES: CC1(C)C2C(=O)N(Cc3cc4c(-c5cc(Cl)ccc5N5C[C@@H]6CCN[C@@H]6C5)ncnn4c3)C(=O)C21 ; Cl] | 3-((4-(5-chloro-2-((3aS,6aS)- hexahydropyrrolo[3,4-b]pyrrol- 5(1H)-yl)phenyl)pyrrolo[2,1- f][1,2,4]triazin-6-yl)methyl)-6,6- dimethyl-3-azabicyclo[3.1.0]hexane- 2,4-dione hydrochloride |
| 97. | 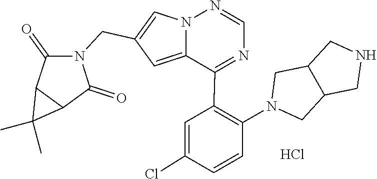 [SMILES: CC1(C)C2C(=O)N(Cc3cc4c(-c5cc(Cl)ccc5N5CC6CNCC6C5)ncnn4c3)C(=O)C21 ; Cl] | 3-((4-(5-chloro-2- (hexahydropyrrolo[3,4-c]pyrrol- 2(1H)-yl)phenyl)pyrrolo[2,1- f][1,2,4]triazin-6-yl)methyl)-6,6- dimethyl-3-azabicyclo[3.1.0]hexane- 2,4-dione hydrochloride |
| 98. | 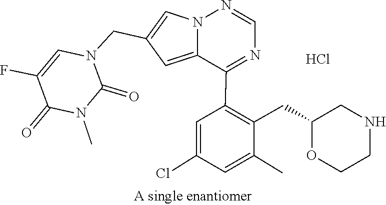 [SMILES: Cc1cc(Cl)cc(-c2ncnn3cc(Cn4cc(F)c(=O)n(C)c4=O)cc23)c1C[C@@H]1CNCCO1 ; Cl] | (R)-1-((4-(5-chloro-3-methyl-2- (morpholin-2- ylmethyl)phenyl)pyrrolo[2,1- f][1,2,4]triazin-6-yl)methyl)-5- fluoro-3-methylpyrimidine- 2,4(1H,3H)-dione hydrochloride |
| 99. | 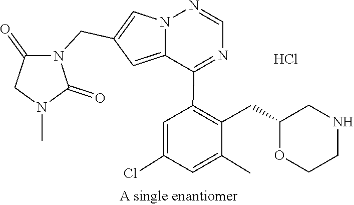 [SMILES: Cc1cc(Cl)cc(-c2ncnn3cc(CN4C(=O)CN(C)C4=O)cc23)c1C[C@@H]1CNCCO1 ; Cl] | (R)-3-((4-(5-chloro-3-methyl-2- (morpholin-2- ylmethyl)phenyl)pyrrolo[2,1- f][1,2,4]triazin-6-yl)methyl)-1- methylimidazolidine-2,4-dione hydrochloride |
| 100. | 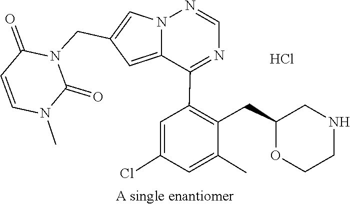 [SMILES: Cc1cc(Cl)cc(-c2ncnn3cc(Cn4c(=O)ccn(C)c4=O)cc23)c1C[C@H]1CNCCO1 ; Cl] | (S)-3-((4-(5-chloro-3-methyl-2- (morpholin-2- ylmethyl)phenyl)pyrrolo[2,1- f][1,2,4]triazin-6-yl)methyl)-1- methylpyrimidine-2,4(1H,3H)-dione hydrochloride |
| 101. | 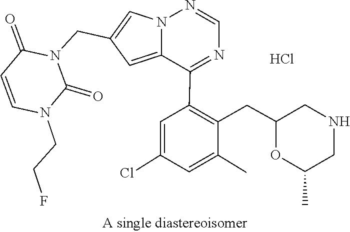 [SMILES: Cc1cc(Cl)cc(-c2ncnn3cc(Cn4c(=O)ccn(CCF)c4=O)cc23)c1CC1CNC[C@H](C)O1 ; Cl] | 3-((4-(5-chloro-3-methyl-2-(((6S)-6- methylmorpholin-2- yl)methyl)phenyl)pyrrolo[2,1- f][1,2,4]triazin-6-yl)methyl)-1-(2- fluoroethyl)pyrimidine-2,4(1H,3H)- dione hydrochloride |
| 102. | 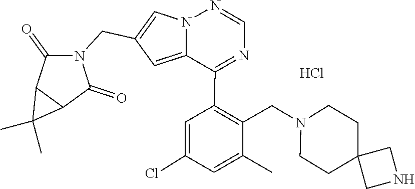 [SMILES: Cc1cc(Cl)cc(-c2ncnn3cc(CN4C(=O)C5C(C4=O)C5(C)C)cc23)c1CN1CCC2(CC1)CNC2 ; Cl] | 3-((4-(2-((2,7-diazaspiro[3.5]nonan- 7-yl)methyl)-5-chloro-3- methylphenyl)pyrrolo[2,1- f][1,2,4]triazin-6-yl)methyl)-6,6- dimethyl-3-azabicyclo[3.1.0]hexane- 2,4-dione hydrochloride |
| 103. | 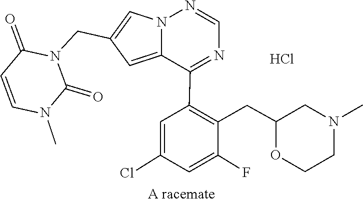 [SMILES: CN1CCOC(Cc2c(F)cc(Cl)cc2-c2ncnn3cc(Cn4c(=O)ccn(C)c4=O)cc23)C1 ; Cl] | 3-((4-(5-chloro-3-fluoro-2-((4- methylmorpholin-2- yl)methyl)phenyl)pyrrolo[2,1- f][1,2,4]triazin-6-yl)methyl)-1- methylpyrimidine-2,4(1H,3H)-dione hydrochloride |
| 104. | 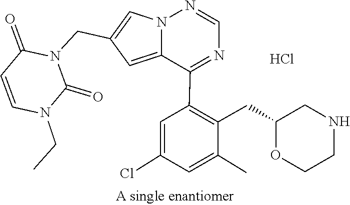 [SMILES: CCn1ccc(=O)n(Cc2cc3c(-c4cc(Cl)cc(C)c4C[C@@H]4CNCCO4)ncnn3c2)c1=O ; Cl] | (R)-3-((4-(5-chloro-3-methyl-2- (morpholin-2- ylmethyl)phenyl)pyrrolo[2,1- f][1,2,4]triazin-6-yl)methyl)-1- ethylpyrimidine-2,4(1H,3H)-dione hydrochloride |
| 105. | 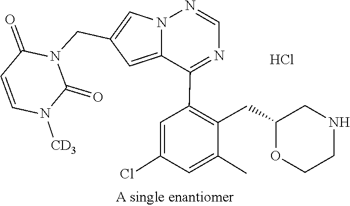 [SMILES: [2H]C([2H])([2H])n1ccc(=O)n(Cc2cc3c(-c4cc(Cl)cc(C)c4C[C@@H]4CNCCO4)ncnn3c2)c1=O ; Cl] | (R)-3-((4-(5-chloro-3-methyl-2- (morpholin-2- ylmethyl)phenyl)pyrrolo[2,1- f][1,2,4]triazin-6-yl)methyl)-1- (methyl-d₃)pyrimidine-2,4(1H,3H)- dione hydrochloride |
| 106. | 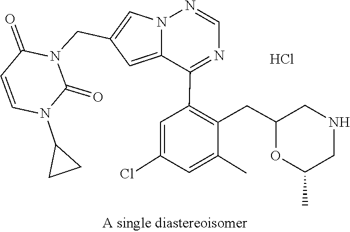 [SMILES: Cc1cc(Cl)cc(-c2ncnn3cc(Cn4c(=O)ccn(C5CC5)c4=O)cc23)c1CC1CNC[C@H](C)O1 ; Cl] | 3-((4-(5-chloro-3-methyl-2-(((6S)-6- methylmorpholin-2- yl)methyl)phenyl)pyrrolo[2,1- f][1,2,4]triazin-6-yl)methyl)-1- cyclopropylpyrimidine-2,4(1H,3H)- dione hydrochloride |
| 107. | 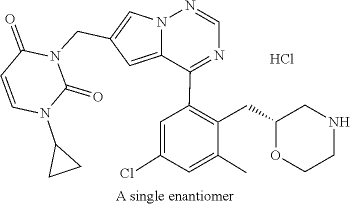 [SMILES: Cc1cc(Cl)cc(-c2ncnn3cc(Cn4c(=O)ccn(C5CC5)c4=O)cc23)c1C[C@@H]1CNCCO1 ; Cl] | (R)-3-((4-(5-chloro-3-methyl-2- (morpholin-2- ylmethyl)phenyl)pyrrolo[2,1- f][1,2,4]triazin-6-yl)methyl)-1- cyclopropylpyrimidine-2,4(1H,3H)- dione hydrochloride |
| 108. | 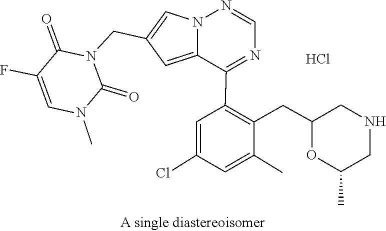 [SMILES: Cc1cc(Cl)cc(-c2ncnn3cc(Cn4c(=O)c(F)cn(C)c4=O)cc23)c1CC1CNC[C@H](C)O1 ; Cl] | 3-((4-(5-chloro-3-methyl-2-(((6S)-6- methylmorpholin-2- yl)methyl)phenyl)pyrrolo[2,1- f][1,2,4]triazin-6-yl)methyl)-5- fluoro-1-methylpyrimidine- 2,4(1H,3H)-dione hydrochloride |
| 109. | 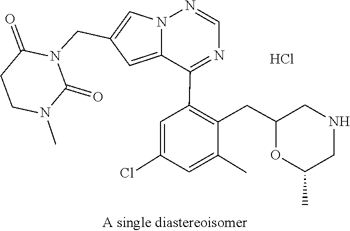 [SMILES: Cc1cc(Cl)cc(-c2ncnn3cc(CN4C(=O)CCN(C)C4=O)cc23)c1CC1CNC[C@H](C)O1 ; Cl] | 3-((4-(5-chloro-3-methyl-2-(((6S)-6- methylmorpholin-2- yl)methyl)phenyl)pyrrolo[2,1- f][1,2,4]triazin-6-yl)methyl)-1- methyldihydropyrimidine- 2,4(1H,3H)-dione hydrochloride |
| 110. | 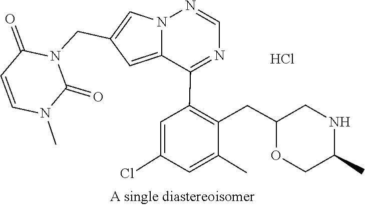 [SMILES: Cc1cc(Cl)cc(-c2ncnn3cc(Cn4c(=O)ccn(C)c4=O)cc23)c1CC1CN[C@@H](C)CO1 ; Cl] | 3-((4-(5-chloro-3-methyl-2-(((5S)-5- methylmorpholin-2- yl)methyl)phenyl)pyrrolo[2,1- f][1,2,4]triazin-6-yl)methyl)-1- methylpyrimidine-2,4(1H,3H)-dione hydrochloride |
| 111. | 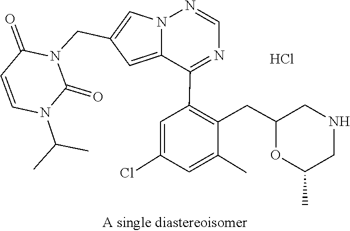 [SMILES: Cc1cc(Cl)cc(-c2ncnn3cc(Cn4c(=O)ccn(C(C)C)c4=O)cc23)c1CC1CNC[C@H](C)O1 ; Cl] | 3-((4-(5-chloro-3-methyl-2-(((6S)-6- methylmorpholin-2- yl)methyl)phenyl)pyrrolo[2,1- f][1,2,4]triazin-6-yl)methyl)-1- isopropylpyrimidine-2,4(1H,3H)- dione hydrochloride |
| 112. | 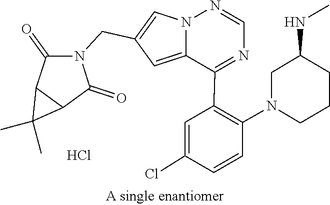 [SMILES: CN[C@H]1CCCN(c2ccc(Cl)cc2-c2ncnn3cc(CN4C(=O)C5C(C4=O)C5(C)C)cc23)C1 ; Cl] | 3-((4-(5-chloro-2-((S)-3- (methylamino)piperidin-1- yl)phenyl)pyrrolo[2,1- f][1,2,4]triazin-6-yl)methyl)-6,6- dimethyl-3-azabicyclo[3.1.0]hexane- 2,4-dione hydrochloride |
| 113. | 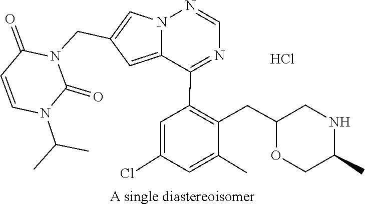 [SMILES: Cc1cc(Cl)cc(-c2ncnn3cc(Cn4c(=O)ccn(C(C)C)c4=O)cc23)c1CC1CN[C@@H](C)CO1 ; Cl] | 3-((4-(5-chloro-3-methyl-2-(((5S)-5- methylmorpholin-2- yl)methyl)phenyl)pyrrolo[2,1- f][1,2,4]triazin-6-yl)methyl)-1- isopropylpyrimidine-2,4(1H,3H)- dione hydrochloride |
| 114. | 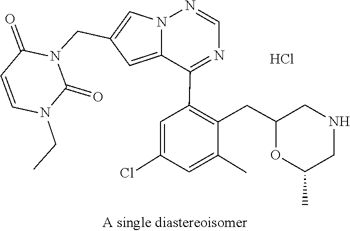 [SMILES: CCn1ccc(=O)n(Cc2cc3c(-c4cc(Cl)cc(C)c4CC4CNC[C@H](C)O4)ncnn3c2)c1=O ; Cl] | 3-((4-(5-chloro-3-methyl-2-(((6S)-6- methylmorpholin-2- yl)methyl)phenyl)pyrrolo[2,1- f][1,2,4]triazin-6-yl)methyl)-1- ethylpyrimidine-2,4(1H,3H)-dione hydrochloride |
| 115. | 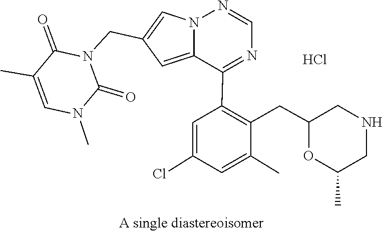 [SMILES: Cc1cc(Cl)cc(-c2ncnn3cc(Cn4c(=O)c(C)cn(C)c4=O)cc23)c1CC1CNC[C@H](C)O1 ; Cl] | 3-((4-(5-chloro-3-methyl-2-(((6S)-6- methylmorpholin-2- yl)methyl)phenyl)pyrrolo[2,1- f][1,2,4]triazin-6-yl)methyl)-1,5- dimethylpyrimidine-2,4(1H,3H)- dione hydrochloride |
| 116. | 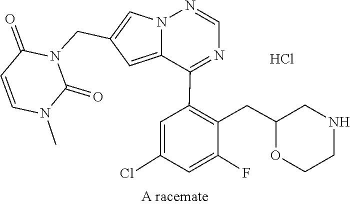 [SMILES: Cn1ccc(=O)n(Cc2cc3c(-c4cc(Cl)cc(F)c4CC4CNCCO4)ncnn3c2)c1=O ; Cl] | 3-((4-(5-chloro-3-fluoro-2- (morpholin-2- ylmethyl)phenyl)pyrrolo[2,1- f][1,2,4]triazin-6-yl)methyl)-1- methylpyrimidine-2,4(1H,3H)-dione hydrochloride |
| 117. | 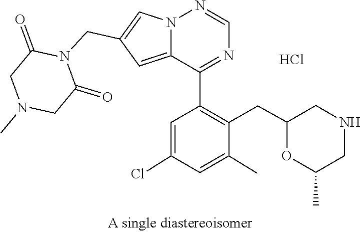 [SMILES: Cc1cc(Cl)cc(-c2ncnn3cc(CN4C(=O)CN(C)CC4=O)cc23)c1CC1CNC[C@H](C)O1 ; Cl] | 1-((4-(5-chloro-3-methyl-2-(((6S)-6- methylmorpholin-2- yl)methyl)phenyl)pyrrolo[2,1- f][1,2,4]triazin-6-yl)methyl)-4- methylpiperazine-2,6-dione hydrochloride |
| 118. | 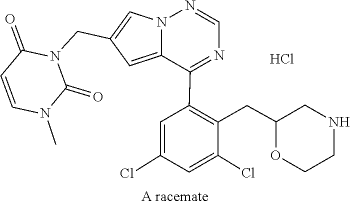 [SMILES: Cn1ccc(=O)n(Cc2cc3c(-c4cc(Cl)cc(Cl)c4CC4CNCCO4)ncnn3c2)c1=O ; Cl] | 3-((4-(3,5-dichloro-2-(morpholin-2- ylmethyl)phenyl)pyrrolo[2,1- f][1,2,4]triazin-6-yl)methyl)-1- methylpyrimidine-2,4(1H,3H)-dione hydrochloride |
| 119. | 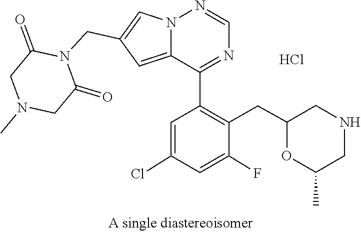 [SMILES: C[C@H]1CNCC(Cc2c(F)cc(Cl)cc2-c2ncnn3cc(CN4C(=O)CN(C)CC4=O)cc23)O1 ; Cl] | 1-((4-(5-chloro-3-fluoro-2-(((6S)-6- methylmorpholin-2- yl)methyl)phenyl)pyrrolo[2,1- f][1,2,4]triazin-6-yl)methyl)-4- methylpiperazine-2,6-dione hydrochloride |
| 120. | 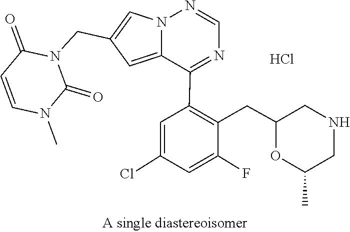 [SMILES: C[C@H]1CNCC(Cc2c(F)cc(Cl)cc2-c2ncnn3cc(Cn4c(=O)ccn(C)c4=O)cc23)O1 ; Cl] | 3-((4-(5-chloro-3-fluoro-2-(((6S)-6- methylmorpholin-2- yl)methyl)phenyl)pyrrolo[2,1- f][1,2,4]triazin-6-yl)methyl)-1- methylpyrimidine-2,4(1H,3H)-dione hydrochloride |
| 121. | 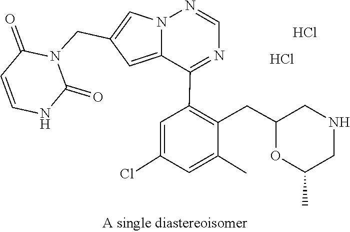 [SMILES: Cc1cc(Cl)cc(-c2ncnn3cc(Cn4c(=O)cc[nH]c4=O)cc23)c1CC1CNC[C@H](C)O1 ; Cl ; Cl] | 3-((4-(5-chloro-3-methyl-2-(((6S)-6- methylmorpholin-2- yl)methyl)phenyl)pyrrolo[2,1- f][1,2,4]triazin-6- yl)methyl)pyrimidine-2,4(1H,3H)- dione dihydrochloride |
| 122. | 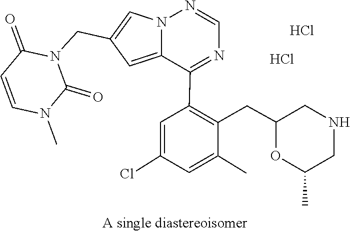 [SMILES: Cc1cc(Cl)cc(-c2ncnn3cc(Cn4c(=O)ccn(C)c4=O)cc23)c1CC1CNC[C@H](C)O1 ; Cl ; Cl] | 3-((4-(5-chloro-3-methyl-2-(((6S)-6- methylmorpholin-2- yl)methyl)phenyl)pyrrolo[2,1- f][1,2,4]triazin-6-yl)methyl)-1- methylpyrimidine-2,4(1H,3H)-dione dihydrochloride |
| 123. | 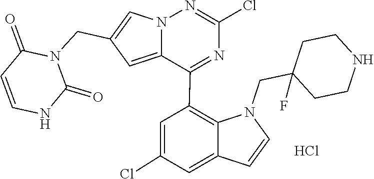 [SMILES: O=c1cc[nH]c(=O)n1Cc1cc2c(-c3cc(Cl)cc4ccn(CC5(F)CCNCC5)c34)nc(Cl)nn2c1 ; Cl] | 3-((2-chloro-4-(5-chloro-1-((4- fluoropiperidin-4-yl)methyl)-1H- indol-7-yl)pyrrolo[2,1- f][1,2,4]triazin-6- yl)methyl)pyrimidine-2,4(1H,3H)- dione hydrochloride |
| 124. | 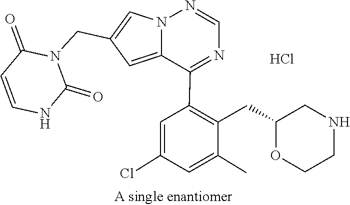 [SMILES: Cc1cc(Cl)cc(-c2ncnn3cc(Cn4c(=O)cc[nH]c4=O)cc23)c1C[C@@H]1CNCCO1 ; Cl] | (R)-3-((4-(5-chloro-3-methyl-2- (morpholin-2- ylmethyl)phenyl)pyrrolo[2,1- f][1,2,4]triazin-6- yl)methyl)pyrimidine-2,4(1H,3H)- dione hydrochloride |
| 125. | 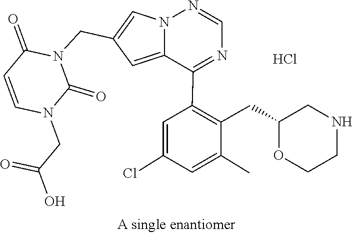 [SMILES: Cc1cc(Cl)cc(-c2ncnn3cc(Cn4c(=O)ccn(CC(=O)O)c4=O)cc23)c1C[C@@H]1CNCCO1 ; Cl] | (R)-2-(3-((4-(5-chloro-3-methyl-2- (morpholin-2- ylmethyl)phenyl)pyrrolo[2,1- f][1,2,4]triazin-6-yl)methyl)-2,4- dioxo-3,4-dihydropyrimidin-1(2H)- yl)acetic acid hydrochloride |
| 126. | 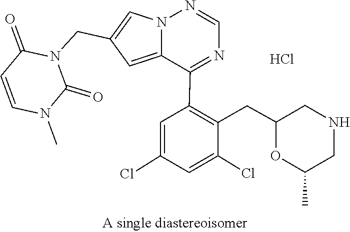 [SMILES: C[C@H]1CNCC(Cc2c(Cl)cc(Cl)cc2-c2ncnn3cc(Cn4c(=O)ccn(C)c4=O)cc23)O1 ; Cl] | 3-((4-(3,5-dichloro-2-(((6S)-6- methylmorpholin-2- yl)methyl)phenyl)pyrrolo[2,1- f][1,2,4]triazin-6-yl)methyl)-1- methylpyrimidine-2,4(1H,3H)-dione hydrochloride |
| 127. | 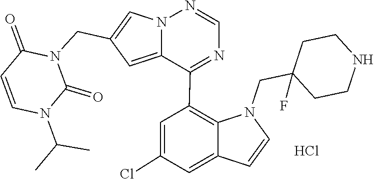 [SMILES: CC(C)n1ccc(=O)n(Cc2cc3c(-c4cc(Cl)cc5ccn(CC6(F)CCNCC6)c45)ncnn3c2)c1=O ; Cl] | 3-((4-(5-chloro-1-((4- fluoropiperidin-4-yl)methyl)-1H- indol-7-yl)pyrrolo[2,1- f][1,2,4]triazin-6-yl)methyl)-1- isopropylpyrimidine-2,4(1H,3H)- dione hydrochloride |
| 128. | 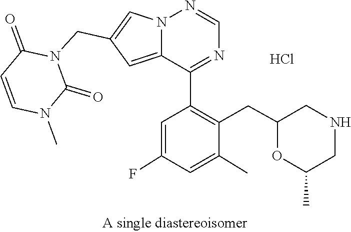 [SMILES: Cc1cc(F)cc(-c2ncnn3cc(Cn4c(=O)ccn(C)c4=O)cc23)c1CC1CNC[C@H](C)O1 ; Cl] | 3-((4-(5-fluoro-3-methyl-2-(((6S)-6- methylmorpholin-2- yl)methyl)phenyl)pyrrolo[2,1- f][1,2,4]triazin-6-yl)methyl)-1- methylpyrimidine-2,4(1H,3H)-dione hydrochloride |
| 129. | 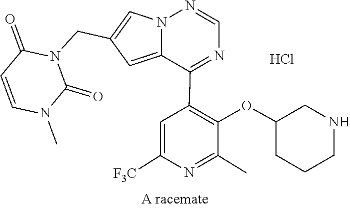 [SMILES: Cc1nc(C(F)(F)F)cc(-c2ncnn3cc(Cn4c(=O)ccn(C)c4=O)cc23)c1OC1CCCNC1 ; Cl] | 1-methyl-3-((4-(2-methyl-3- (piperidin-3-yloxy)-6- (trifluoromethyl)pyridin-4- yl)pyrrolo[2,1-f][1,2,4]triazin-6- yl)methyl)pyrimidine-2,4(1H,3H)- dione hydrochloride |
| 130. | 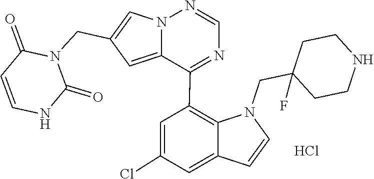 [SMILES: O=c1cc[nH]c(=O)n1Cc1cc2c(-c3cc(Cl)cc4ccn(CC5(F)CCNCC5)c34)ncnn2c1 ; Cl] | 3-((4-(5-chloro-1-((4- fluoropiperidin-4-yl)methyl)-1H- indol-7-yl)pyrrolo[2,1- f][1,2,4]triazin-6- yl)methyl)pyrimidine-2,4(1H,3H)- dione hydrochloride |
| 131. | 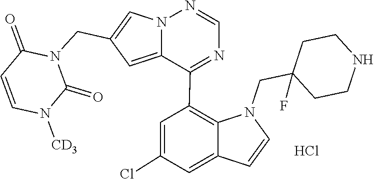 [SMILES: [2H]C([2H])([2H])n1ccc(=O)n(Cc2cc3c(-c4cc(Cl)cc5ccn(CC6(F)CCNCC6)c45)ncnn3c2)c1=O ; Cl] | 3-((4-(5-chloro-1-((4- fluoropiperidin-4-yl)methyl)-1H- indol-7-yl)pyrrolo[2,1- f][1,2,4]triazin-6-yl)methyl)-1- (methyl-d₃)pyrimidine-2,4(1H,3H)- dione hydrochloride |
| 132. | 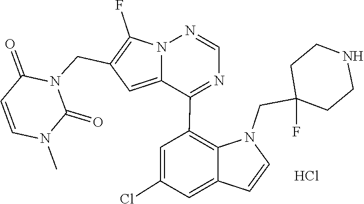 [SMILES: Cn1ccc(=O)n(Cc2cc3c(-c4cc(Cl)cc5ccn(CC6(F)CCNCC6)c45)ncnn3c2F)c1=O ; Cl] | 3-((4-(5-chloro-1-((4- fluoropiperidin-4-yl)methyl)-1H- indol-7-yl)-7-fluoropyrrolo[2,1- f][1,2,4]triazin-6-yl)methyl)-1- methylpyrimidine-2,4(1H,3H)-dione hydrochloride |

It will be readily appreciated that the invention also encompasses the free base compounds of the specific acid addition salts shown above. Again, other pharmaceutically acceptable salts of these free bases are also within the scope of the invention. The invention also encompasses tautomers, stereoisomers, racemic and scalemic mixtures of stereoisomers, esters, solvates, and polymorphs of the compound according to the invention.

### Pharmaceutical Compositions of the Invention

In another aspect, the invention provides a pharmaceutical composition comprising (i) a therapeutically effective amount of at least one compound according to the invention, or a pharmaceutically acceptable salt, solvate, or polymorph thereof, and (ii) a pharmaceutically acceptable carrier, vehicle or excipient therefor. In general, pharmaceutical compositions comprise (i) a therapeutically effective amount of at least one compound of the invention, or a tautomer, stereoisomer, pharmaceutically acceptable salt, and/or a solvate thereof; and (ii) a pharmaceutically acceptable carrier, vehicle or excipient therefor, including, but not limited to, bioavailability enhancers, penetration enhancers, biopolymers, PLGA-based nanoparticles, sugar-based nanoparticles, coating to avoid the deleterious effects of the stomach environment, either by protection of the compound of the invention or derivative thereof, or by release of the biologically active material beyond the stomach environment, such as in the intestine.

The exact nature of the carrier, or, for example excipient or diluent, will depend upon the desired use for the composition, and may be suitable or acceptable for veterinary use and/or suitable or acceptable for human use. The composition may optionally include one or more additional compounds, including one or more additional therapeutic agents.

Compounds of the invention can be combined with other therapeutic agents. The compound of the invention and other therapeutic agent may be administered simultaneously or sequentially. When the other therapeutic agents are administered simultaneously, they can be administered in the same or separate formulations, but they are administered substantially at the same time. The other therapeutic agents are administered sequentially with one another and with compound of the invention, when the administration of the other therapeutic agents and the compound of the invention is temporally separated. The separation in time between the administration of these compounds may be a matter of minutes or it may be longer.

Examples of other therapeutic agents that may be administered with the compounds of the invention include steroids, membrane stabilizers, 5LO inhibitors, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, rituximab, p38 inhibitors, PDE4 inhibitors, and antihistamines, immunotherapeutic agents, including checkpoint inhibitors such as PD-1, PD-L1, CTLA-4, LAG-3, TIM-3, TIGIT, VISTA inhibitors, IDO/TDO inhibitors, Arg1 and Arg2 inhibitors, adenosine A2A receptor antagonists, ectonucleotidase (CD73 and CD39) inhibitors, immunosuppressants, agents affecting interleukins, cytokines and chemokines, kinase inhibitors, chemotherapeutic agents including alkylating antineoplastic agents, antimetabolites, anti-microtubule agents, topoisomerase inhibitors, cytotoxic antibiotics or targeted therapies such as antibodies, antibodies drug conjugates, cell-based immunotherapies, nanoparticles, anti-cancer vaccines and radiotherapy.

In some embodiments, the one or more additional chemotherapeutic agents includes aminoglutethimide, amsacrine, anastrozole, asparaginase, AZD5363, Bacillus Calmette-Guerin vaccine (BCG), bicalutamide, bleomycin, bortezomib, buserelin, busulfan, camptothecin, capecitabine, carboplatin, carfilzomib, carmustine, chlorambucil, chloroquine, cisplatin, cladribine, clodronate, cobimetinib, colchicine, cyclophosphamide, cyproterone, cytarabine, dacarbazine, dactinomycin, daunorubicin, demethoxyviridin, dexamethasone, dichloroacetate, dienestrol, diethylstilbestrol, docetaxel, doxorubicin, epirubicin, erlotinib, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, miltefosine, mitomycin, mitotane, mitoxantrone, MK-2206, nilutamide, nocodazole, octreotide, olaparib, oxaliplatin, paclitaxel, pamidronate, pazopanib, pentostatin, perifosine, plicamycin, pomalidomide, porfimer, procarbazine, raltitrexed, rituximab, rucaparib, selumetinib, sorafenib, streptozocin, sunitinib, suramin, talazoparib, tamoxifen, temozolomide, temsirolimus, teniposide, testosterone, thalidomide, thioguanine, thiotepa, titanocene dichloride, topotecan, trametinib, trastuzumab, tretinoin, veliparib, vinblastine, vincristine, vindesine, or vinorelbine.

In some embodiments, the one or more additional chemotherapeutic agents include abagovomab, adecatumumab, afutuzumab, anatumomab mafenatox, apolizumab, avelumab, blinatumomab, catumaxomab, durvalumab, epratuzumab, inotuzumab ozogamicin, intelumumab, ipilimumab, isatuximab, lambrolizumab, nivolumab, ocaratuzumab, olatatumab, pembrolizumab, pidilizumab, ticilimumab, samalizumab, tremelimumab, and BMS-936559.

In other embodiments, the method further comprises administering one or more non-chemical methods of cancer treatment, such as radiation therapy, surgery, thermoablation, focused ultrasound therapy, cryotherapy, or a combination thereof.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of active compounds will be, for human subjects, from about 0.0001 milligrams/kg per day, 0.001 milligrams/kg per day, or 0.01 milligrams/kg per day to about 100 milligrams/kg per day or 1000 milligrams/kg per day. It is expected that oral doses in the range of 0.5 to 50 milligrams/kg, in one or several administrations per day, will yield the desired results. Dosage may be adjusted appropriately to achieve desired drug levels sufficient to achieve or maintain a desired therapeutic effect, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds. The compounds may be administered once per week, several times per week (e.g., every other day), once per day or multiple times per day, depending upon, among other things, the mode of administration, the specific indication being treated and the judgment of the prescribing physician.

In one embodiment, intravenous administration of a compound of the invention may typically be from 0.1 mg/kg/day to 20 mg/kg/day.

Determination of an effective dosage of a compound for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. For any compound described herein the therapeutically effective amount can be initially determined from animal models. A therapeutically effective dose can also be determined from human data for compounds of the invention which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

Pharmaceutical compositions comprising the compound of the invention may be manufactured by means of conventional mixing, dissolving, granulating, dragée-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

For use in therapy, an effective amount of the compound of the invention can be administered to a subject by any mode that delivers the compound of the invention to the desired surface. Administering the pharmaceutical composition of the present invention may be accomplished by any means known to the skilled artisan. Routes of administration include but are not limited to oral, buccal, nasal, rectal, vaginal, ocular, topical, intravenous, intramuscular, intraperitoneal, subcutaneous, transdermal, direct injection (for example, into an abscess), mucosal, inhalation, and insufflation.

For oral administration, the compounds (i.e., compounds of the invention, and other therapeutic agents) can be formulated readily by combining the active compound(s) with pharmaceutically acceptable carriers well known in the art. Such carriers enable the compounds of the invention to be formulated as tablets, pills, dragées, lozenges, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragée cores. Suitable excipients are, in particular, binding agents, fillers, lubricants, disintegrants, and wetting agents. Suitable fillers include sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal, oral or pulmonary administration.

For administration by inhalation, the compounds for use according to the present invention may be conveniently delivered in the form of an aerosol spray presentation from pressurized packs or a nebulizer, with the use of a suitable propellant, e.g., dichlorodifluoro-methane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds of the invention (or derivatives thereof). The compound of the invention (or derivative) is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (α1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of compound of the invention (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise compound of the invention (or derivative) dissolved in water at a concentration of about 0.1 to 25 mg of biologically active compound of the invention per mL of solution. The formulation may also include a buffer and a simple sugar (e.g., for compound of the invention stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as sterile suspensions, solutions, or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, buffer, dextrose solution, before use. To this end, the active compound may be dried by any art-known technique, such as lyophilization, and reconstituted prior to use.

The compounds may also be formulated in rectal or vaginal compositions such as suppositories or retention enemas, e.g., containing conventional suppository bases such as cocoa butter or other glycerides.

For transmucosal administration, penetrants appropriate to the barrier to be permeated are used in the formulation. Such penetrants are known in the art.

For ocular administration, the compound(s) may be formulated as a solution, emulsion, suspension, etc. suitable for administration to the eye. A variety of vehicles suitable for administering compounds to the eye are known in the art.

In addition to the formulations described above, for prolonged delivery, the compounds may also be formulated as a depot preparation for administration by, for example, implantation or intramuscular injection. Such long acting formulations may be formulated with suitable polymeric or hydrophobic materials (for example as an emulsion in an acceptable oil) or ion exchange resins, or as sparingly soluble derivatives, for example, as a sparingly soluble salt. Alternatively, transdermal delivery systems manufactured as an adhesive disc or patch which slowly releases the compound for percutaneous absorption may be used. To this end, permeation enhancers may be used to facilitate transdermal penetration of the compound.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990), which is incorporated herein by reference.

The compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulfuric, nitric, phosphoric, maleic, acetic, salicylic, p-toluenesulfonic, tartaric, citric, methane sulfonic, formic, malonic, succinic, naphthalene-2-sulfonic, and benzenesulfonic. Also, such salts can be prepared as alkaline metal or alkaline earth salts, such as sodium, potassium or calcium salts of the carboxylic acid group. Typically, such salts are more soluble in aqueous solutions than the corresponding free acids and bases, but salts having lower solubility than the corresponding free acids and bases may also be formed.

The compounds may alternatively be formulated in the pharmaceutical composition per se, or in the form of a hydrate, solvate, or N-oxide.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound of the invention and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to the compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, non-erodible, biodegradable, or non-biodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bio-erodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions, methods, and uses described herein are readily apparent from the description of the invention contained herein in view of information known to the ordinarily skilled artisan, and may be made without departing from the scope of the invention or any embodiment thereof.

### Methods and Uses

As shown herein, the compounds of the invention are useful for inhibiting the enzymatic and biological activity of ubiquitin specific protease 7.

Another aspect of the invention is a method for inhibiting USP7 in a cell or a tissue, comprising contacting the cell or the tissue with at least one compound according to the invention, or a pharmaceutically acceptable salt, solvate, or polymorph thereof, or with a pharmaceutical composition according to the invention.

In another aspect, the invention provides a method for the treatment or prevention of a disease, disorder, or condition associated with aberrant expression or activity of USP7, comprising administering to the subject in need thereof a therapeutically effective amount of at least one compound according to the invention, or a pharmaceutically acceptable salt, solvate, or polymorph thereof, or a pharmaceutical composition according to the invention.

In certain embodiments, the disease, disorder, or condition is selected from the group consisting of cardiovascular disorders, pulmonary disorders, autoimmune disorders, immune disorders, immunoregulatory disorders, neurodegenerative disorders, metabolic disorders, hemolytic disorders, gastrointestinal disorders, sexual disorders, infections, wound healing disorders, and cancers.

In certain embodiments, the invention provides a compound of Formula (I) for use in a method for inhibiting USP7 in a cell or a tissue, comprising contacting the cell or the tissue with at least one such compound, or a pharmaceutically acceptable salt, solvate, or polymorph thereof.

In some aspects, the invention provides use of a compound according to the invention, or a pharmaceutically acceptable salt, solvate, or polymorph thereof, in the manufacturing of a medicament for the treatment of a disease, disorder, or condition associated with expression of USP7.

Additionally, the invention provides a compound according to the invention, or a pharmaceutically acceptable salt, solvate, or polymorph thereof, for use in a method for the treatment or prevention of a disease, disorder, or condition associated with aberrant expression or activity of USP7, comprising administering to the subject in need thereof a therapeutically effective amount of at least one compound according to the invention, or a pharmaceutically acceptable salt, solvate, or polymorph thereof, or a pharmaceutical composition according to the invention.

In certain embodiments, the disease, disorder, or condition is selected from the group consisting of cardiovascular disorders, pulmonary disorders, autoimmune disorders, immune disorders, immunoregulatory disorders, neurodegenerative disorders, metabolic disorders, hemolytic disorders, gastrointestinal disorders, sexual disorders, infections, wound healing disorders, and cancers.

In certain embodiments, the disease, disorder, or condition is a cardiovascular disorder selected from the group consisting of systemic hypertension, pulmonary arterial hypertension (PAH), pulmonary arterial hypertension in high altitude, ischemia reperfusion (IR) injury, myocardial infarction, and atherosclerosis.

In certain embodiments, the cardiovascular disorder is pulmonary arterial hypertension (PAH).

In certain embodiments, the cardiovascular disorder is ischemia reperfusion (IR) injury selected from the group consisting of liver IR, kidney IR, and myocardial IR.

In certain embodiments, the cardiovascular disorder is myocardial infarction or atherosclerosis.

In certain embodiments, the disease, disorder, or condition is a pulmonary disorder selected from the group consisting of chemically-induced lung fibrosis, idiopathic pulmonary fibrosis, cystic fibrosis, chronic obstructive pulmonary disease (COPD), and asthma.

In certain embodiments, the disease, disorder, or condition is an autoimmune disorder selected from the group consisting of encephalomyelitis, multiple sclerosis, anti-phospholipid syndrome 1, autoimmune hemolytic anemia, chronic inflammatory demyelinating polyradiculoneuropathy, psoriasis, dermatitis herpetiformis, dermatomyositis, myasthenia gravis, pemphigus, rheumatoid arthritis, stiff-person syndrome, type 1 diabetes, ankylosing spondylitis, paroxysmal nocturnal hemoglobinuria (PNH), paroxysmal cold hemoglobinuria, severe idiopathic autoimmune hemolytic anemia, and Goodpasture's syndrome.

In certain embodiments, the disease, disorder, or condition is an immune disorder selected from the group consisting of T-cell dysfunction mediated by myeloid-derived suppressor cells (MDSC), human immunodeficiency virus (HIV) infection, autoimmune encephalomyelitis, and ABO mismatch transfusion reaction.

In certain embodiments, the immune disorder is T-cell dysfunction mediated by myeloid-derived suppressor cells (MDSC).

In certain embodiments, the disease, disorder, or condition is a disease resulting from an immunoregulatory disorder selected from the group consisting of renal disease inflammation, hepatic fibrosis, leishmaniosis, neurodegenerative diseases, wound healing, human immunodeficiency virus (HIV) infection, hepatitis B virus (HBV) infection, Helicobacter pylori infection, fibrotic disorders, arthritis, candidiasis, periodontal disease, keloids, adenotonsilar disease, African sleeping sickness, Chagas' disease, and transplant rejection.

In certain embodiments, the disease, disorder, or condition is a neurodegenerative disorder selected from the group consisting of Alzheimer's disease, Parkinson's disease, Huntington's disease, extrapyramidal syndrome, dystonia, akathisia, epilepsy, periodic limb movement, and dementia.

In certain embodiments, the disease, disorder, or condition is a metabolic disorder selected from the group consisting of diabetes, non-alcoholic steatohepatitis (NASH), and non-alcoholic fatty liver disease (NAFLD).

In certain embodiments, the disease, disorder, or condition is a hemolytic disorder selected from the group consisting of sickle-cell disease, thalassemias, hereditary spherocytosis, stomatocytosis, microangiopathic hemolytic anemias, pyruvate kinase deficiency, infection-induced anemia, cardiopulmonary bypass, mechanical heart valve-induced anemia, and chemical-induced anemia.

In certain embodiments, the hemolytic disorder is sickle-cell disease.

In certain embodiments, the disease, disorder, or condition is a gastrointestinal disorder selected from the group consisting of gastrointestinal motility disorders, gastric cancers, inflammatory bowel disease, Crohn's disease, ulcerative colitis, and gastric ulcers.

In certain embodiments, the disease, disorder, or condition is a sexual disorder selected from the group consisting of Peyronie's disease, and erectile dysfunction.

In certain embodiments, the disease, disorder, or condition is a wound healing disorder selected from the group consisting of infected and uninfected wound healing.

In certain embodiments, the disease, disorder, or condition is a cancer selected from the group consisting of oesophagic, gastric, colon, ovary, breast, pancreatic, head-and-neck, bladder, and lung cancers (including squamous and non-small cell lung carcinoma), renal cell carcinoma, prostate carcinoma, multiple myeloma, neuroblastoma, glioblastoma, astrocytoma, mesothelioma and melanoma, B cells, T cells and NK cells lymphomas, acute and chronic, myeloid leukemia, and lymphoid leukemia.

In certain embodiments, the disease, disorder, or condition is a cancer selected from the group consisting of gastric cancer (including, but not limited to, gastric or gastroesophageal junction cancer), colorectal cancer, pancreatic cancer, liver cancer, breast cancer, lung cancers (including, but not limited to, non-small cell lung carcinoma), renal cell carcinoma, prostate carcinoma, multiple myeloma, acute and chronic leukemias, T cell, B cell and NK cell lymphomas, brain tumors (including, but not limited to, neuroblastoma, glioblastoma, astrocytoma), squamous-cell carcinomas of the head and neck, and melanoma.

In certain embodiments, the disease, disorder, or condition is a cancer selected from the group consisting of chronic lymphocytic leukemia, acute lymphoblastic leukemia, chronic myelogenous leukemia, acute myeloid leukemia, adrenocortical carcinoma, anal cancer, appendix cancer, atypical teratoid/rhabdoid tumor, basal cell carcinoma, bile duct cancer, bladder cancer, bone cancer, brain tumor, brain and spinal cord tumor, brain stem glioma, central nervous system atypical teratoid/rhabdoid tumor, central nervous system embryonal tumors, breast cancer, bronchial tumors, Burkitt lymphoma, carcinoid tumor, carcinoma of unknown primary, central nervous system cancer, cervical cancer, childhood cancers, chordoma, chronic myeloproliferative disorders, colon cancer, craniopharyngioma, cutaneous T-cell lymphoma, ductal carcinoma in situ, embryonal tumors, endometrial cancer, ependymoblastoma, ependymoma, esophageal cancer, esthesioneuroblastoma, Ewing sarcoma, extracranial germ cell tumor, extrahepatic bile duct cancer, eye cancer, fibrous histiocytoma of bone, gallbladder cancer, gastric cancer, gastrointestinal carcinoid tumor, gastrointestinal stromal tumors, germ cell tumor, extracranial germ cell tumor, extragonadal germ cell tumor, ovarian germ cell tumor, gestational trophoblastic tumor, glioma, hairy cell leukemia, head and neck cancer, heart cancer, hepatocellular cancer, histiocytosis, Langerhans cell cancer, Hodgkin's lymphoma, hypopharyngeal cancer, intraocular melanoma, islet cell tumors, Kaposi sarcoma, kidney cancer, Langerhans cell histiocytosis, laryngeal cancer, lip and oral cavity cancer, lobular carcinoma in situ, lymphoma, AIDS-related lymphoma, macroglobulinemia, male breast cancer, medulloblastoma, medulloepithelioma, Merkel cell carcinoma, malignant mesothelioma, metastatic squamous neck cancer, midline tract carcinoma involving NUT gene, mouth cancer, multiple endocrine neoplasia syndrome, plasma cell neoplasm, mycosis fungoides, myeloma, chronic myeloproliferative disorder, nasal cavity cancer, paranasal sinus cancer, nasopharyngeal cancer, non-Hodgkin's lymphoma, oral cancer, oral cavity cancer, lip cancer, oropharyngeal cancer, osteosarcoma, ovarian cancer, papillomatosis, paraganglioma, paranasal sinus cancer, nasal cavity cancer, parathyroid cancer, penile cancer, pharyngeal cancer, pheochromocytoma, pineal parenchymal tumors of intermediate differentiation, pineoblastoma, pituitary tumor, plasma cell neoplasm, pleuropulmonary blastoma, primary central nervous system lymphoma, rectal cancer, renal cell cancer, renal pelvis cancer, ureter cancer, transitional cell cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, squamous neck cancer with occult primary, supratentorial primitive neuroectodermal tumors, testicular cancer, throat cancer, thymoma, thymic carcinoma, thyroid cancer, transitional cell cancer of the renal pelvis and ureter, gestational trophoblastic tumor, unknown primary, unusual cancer of childhood, urethral cancer, uterine cancer, uterine sarcoma, Waldenströms macroglobulinemia, or Wilms' tumor.

In certain embodiments, the at least one compound according to the invention is administered simultaneously or sequentially with a therapeutically effective amount of one or more other therapeutic agent(s) selected from the group consisting of anti-viral agents, chemotherapeutic agents (including alkylating antineoplastic agents, antimetabolites, anti-microtubule agents), immunosuppressants, anti-tumor vaccines, antiviral vaccines, cytokine therapy, tyrosine kinase inhibitors, immunotherapeutic agents, including checkpoint inhibitors such as PD-1, PD-L1 or CTLA-4 inhibitors and IDO/TDO inhibitors, adenosine A2A receptor antagonists, ectonucleotidase (CD73 and CD39) inhibitors, agent affecting interleukins, cytokines and chemokines, topoisomerase inhibitors, and cytotoxic antibiotics, or targeted therapies comprising antibodies, antibody drug conjugates, cell-based immunotherapy, nanoparticles, and radiotherapy.

In certain embodiments, the antibodies comprise a therapeutically effective amount of anti-PD-1, anti-PD-L1 or anti-CTLA4 antibodies.

In another aspect, the invention provides use of a compound according to the invention, or a pharmaceutically acceptable salt, solvate, or polymorph thereof, for protecting an organ during transport.

In certain embodiments, the subject is a mammal selected from the group consisting of human, dog, cat, horse, cow, pig, sheep, goat, and ape.

### EXAMPLES

The present invention is further illustrated by the following examples, which in no way should be construed as limiting the scope of the claimed invention.

### Methods of Preparation and Characterization

The compounds of the present disclosure may be prepared by use of known chemical reactions and procedures. Representative methods for synthesizing compounds of the disclosure are presented below. It is understood that the nature of the substituents required for the desired target compound often determines the preferred method of synthesis. All variable groups of these methods are as described in the generic description if they are not specifically defined below. The meaning of the symbols is limited to a particular reaction scheme and is not necessarily the same for all the structural formulas.

Those having skill in the art will recognize that the starting materials and reaction conditions may be varied, the sequence of the reactions altered, and additional steps employed to produce compounds encompassed by the present disclosure, as demonstrated by the following examples. Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-lnterscience, 2001; or Vogel, A Textbook of Practical Organic Chemistry, Including Qualitative Organic Analysis, Fourth Edition, New York: Longman, 1978).

The reactions are performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure.

In some cases, protection of certain reactive functionalities may be necessary to achieve some of the above transformations. In general, the need for such protecting groups as well as the conditions necessary to attach and remove such groups will be apparent to those skilled in the art of organic synthesis. An authoritative account describing the many alternatives to the trained practitioner are in J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides;” Volume 3 (editors: E. Gross and J. Meienhofer), Academic Press, London and New York 1981, in “Methoden der organischen Chemie,” Houben-Weyl, 4th edition, Vol. 15/1, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide und Derivate,” Georg Thieme Verlag, Stuttgart 1974. The protecting groups may be removed at a convenient subsequent stage using methods known from the art. The disclosures of all articles and references mentioned in this application, including patents, are incorporated herein by reference in their entirety.

Starting materials can be obtained from commercial sources or prepared by well-established literature methods known to those skilled in the art.

All solvents, substrates and reagents that were commercially available were used without further purification. TLC analysis was performed using pre-coated glass plates (0.2±0.03 mm thickness, GF-254, particle size 0.01-0.04 mm) from Fluorochem Ltd, UK or using pre-coated glass plates (TLC silica gel 60 F₂₅₄) od using pre-coated aluminium plates (TLC silica gel 60 F₂₅₄) from Merck. TLC analysis was performed for compounds before their transferring in the appropriate salts (hydrochloride or TFA salt).

The column chromatography was performed using high-purity grade silica gel (pore size 60 Å, 220-440 mesh particle size, 35-75 μm particle size) from Fluka or using high-purity grade silica gel (pore size 60 Å, 230-400 mesh particle size, 40-63 μm particle size) from Merck.

Column chromatography (LCC) was performed using high-purity grade silica gel (pore size 60 Å, 220-440 mesh particle size, 35-75 μm particle size) purchased from Fluka.

Flash column chromatography (FLCC) was performed using Buchi Reverelis® X2-UV equipped in evaporative light scattering detector (ELSD), with UV detection in range UV of 200-500 nm, purchased from BÜCHI Labortechnik AG, using puriFlash® pre-packed cartridges with amorphous or spherical virgin silica gel, 50 μm, from InterChim®.

Preparative HPLC were performed on preparative Shimadzu HPLC system containing two pumps LC-20AP, CBM-20A communication module and ELSD-LTII detector with the use of:
Phenomenex Luna 21.2/250 mm, 5 μm C-18(2) 100 Å column;
Thermo Scientific Hypersil GOLD 21.2/250 mm, 5 μm C-18 column;
Phenomenex Luna® 250×30 mm, 5 μm Phenyl-Hexyl 100 Å column;
Phenomenex Luna 250×21.2 mm, 5 μm Phenyl-Hexyl 100 Å column;
Phenomenex Lux® 250×21.2 mm Cellulose-4 column;
   or on preparative Shimadzu HPLC system containing LC-20AP pumps, CBM-20A communication module and SPD-20A UV/VIS detector and FRC-20 fraction collector with the use of:
Phenomenex Luna 250×21.2 mm, 5 μm Phenyl-Hexyl 100 Å column;
Phenomenex Luna® 250×30 mm, 5 μm Phenyl-Hexyl 100 Å column.

The target compounds, when subjected to reversed-phase chromatographic purification in the presence of HCl or TFA, were usually obtained in the form of HCl or TFA salts. The preparative reversed-phase column chromatography was performed using Luna 21.2/250 mm, 5 μm C18(2) 100 ALC column or Cosmosil Cholester 250×20 mm column.

¹H and ¹⁹F NMR spectra were recorded on Bruker AVANCE II PLUS (Ultra Shield) NMR spectrometer at 700 MHz and 250 MHz.

All spectra were recorded in appropriate deuterated solvents (CDCl₃, DMSO-d₆, D₂O, Methanol-d₄, etc.) that were commercially available.

Resonances are given in parts per million (ppm) relative to tetramethylsilane internal standard. Data are reported as follows: chemical shift (δ scale), multiplicity (s=singlet, d=doublet, t=triplet, m=multiplet, bs=broad singlet), coupling constants (J in Hz) and integration.

ESI-MS spectra were obtained on a Shimadzu LC-20AD LPG separation module with a SPD-M20A UV detector and LCMS-2020 mass detector equipped with Phenomenex Kinetex 30×2.1 mm, 1.7 μm XB-C18 100 Å LC column eluted with 1 mL/min flow of 10-90% gradient (over 2 min) then 90% (over 1 min) of acetonitrile with 0.1% HCOOH and water (Method A) or eluted with 1 mL/min flow of 0-50% gradient (over 2 min) then 90% (over 1 min) of acetonitrile with 0.1% HCOOH and water (Method B).

ESI-MS spectra were obtained on a Shimadzu LC-20AD LPG separation module with a SPD-M20A UV detector and LCMS-2020 mass detector equipped with Kinetex 2.1/30 mm, 1.7 μm XB-C18 100 Å LC column eluted with 1 mL/min flow of 10-90% gradient (over 4 min) of acetonitrile in water (Method C).

ESI-MS spectra were obtained on a Shimadzu LC-20AD LPG separation module with a SPD-M20A UV detector and LCMS-2020 mass detector equipped with Kinetex 2.1/30 mm, 1.7 μm XB-C18 100 Å LC column eluted with 0.5 mL/min flow of 10-90% gradient (over 8 min) of acetonitrile in water (Method D).

ESI-MS spectra were obtained on a Shimadzu LC-20AD LPG separation module with a SPD-M20A UV detector and LCMS-2020 mass detector equipped with Phenomenex Kinetex 30×2.1 mm, 1.7 μm XB-C18 100 Å LC column eluted with 1 mL/min flow of 10-90% gradient (over 10 min) then 90% (over 4 min) of acetonitrile with 0.1% HCOOH and water (Method E).

Abbreviations used are those conventional in the art or the following: Ac=acetyl, AcOEt=ethyl acetate, Boc=tert-butoxycarbonyl, BSA=bovine serum albumin, tBu=tert-butyl, ° C.=degree Celsius, DAST=diethylaminosulfur trifluoride, DCM=dichloromethane, DIAD=diisopropyl azodicarboxylate, DIBAL-H=diisobutylaluminium hydride, DIPEA=N,N-diisopropylethylamine, DMAP=4-dimethylaminopyridine, DMF=N,N-dimethylformamide, DMS=dimethyl sulfide, DMSO=dimethyl sulfoxide, dppf=1,1′-bis(diphenylphosphino)ferrocene, DTT=1,4-dithiothreitol, EDCI=1-ethyl-3-(3-dimethylaminopropyl)carbodiimide, ELSD=evaporative light scattering detector, ESI-MS=electrospray ionization mass spectrometry, Et3N=triethylamine, FLCC=flash column chromatography, g=gram, h=hour(s), HATU=1-[bis(dimethylamino)methylene]-1H-1,2,3-triazolo[4,5-b]pyridinium 3-oxide hexafluorophosphate, HEPES=N-(2-hydroxyethyl)piperazine-N-(2-ethanesulfonic acid), HPLC=high pressure liquid chromatography, Hz=hertz, K=kelvin, L=liter, LCC=liquid column chromatography, LC-MS=liquid chromatography and mass spectrometry, M=molar, MeCN=acetonitrile, MeOH=methanol, mg=milligram(s), MHz=megahertz, min=minutes, mL=milliliter(s), mmol=millimoles, MOMCl=chloromethyl methyl ether, Ms=mesyl, m/z=mass to charge ratio, μg=microgram, μL=microliter, μM=micromolar, nM=nanomolar, nm=nanometer, NMM=N-methylmorpholine, NMR=nuclear magnetic resonance, N=normal, PPh₃=triphenylphosphine, RPM=rotation per minute, RT or rt=room temperature, Rₜ=retention time, TFA=trifluoroacetic acid, THE=tetrahydrofuran, TLC=thin layer chromatography, and UPLC=ultra performance liquid chromatography.

If not otherwise defined, purity of a solid substance is expressed as a ratio of the weight of the component in question to the total weight, multiplied by 100 (weight %); purity of a liquid is expressed as a ratio of the volume of the component in question to the total volume, multiplied by 100 (volume %); concentration of a solution is expressed as a ratio of the weight of the solute (in grams) to the total volume (in mL) of the solution, multiplied by 100 (w/v %). Yield of a reaction is expressed as a ratio of the weight of the product in question to the theoretical yield of this product, multiplied by 100(%). Composition of a mixed solvent is expressed as a proportion of volume parts of the component solvents (e.g., 3:1).

### General Synthetic Procedures

### General Procedure I

Reaction of an Appropriate Carboxylic Acid with an Appropriate Amine or Alcohol.

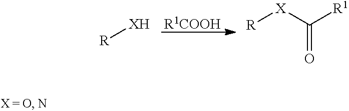

To the solution of a carboxylic acid (1 equivalent) and diisopropylethylamine (DIPEA; 3 equivalents) or triethylamine (Et₃N; 3 equivalents) in dichloromethane (6 mL/mmol) or in DMF (5 mL/mmol) appropriate alcohol (1-1.2 equivalent) or amine (1 equivalent) was added. Then EDCI hydrochloride (1.1-1.5 equivalent) or HATU (1.1 equivalent) and DMAP (0.05-0.1 equivalent, when it was necessary) were added sequentially at 0° C. and then the reaction mixture was stirred overnight at room temperature. After this time LC-MS control showed complete consumption of the starting materials and the reaction mixture was taken into DCM/water. An organic layer was washed with water and brine, dried over anhydrous MgSO₄, filtered and concentrated in vacuo and the crude product was further purified by flash column chromatography on silica.

### General Procedure II

Protection of Amino Group with Di-Tert-Butyl Dicarbonate (Boc₂O).

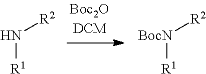

To the solution of appropriate amine (1 equivalent) in DCM or THE (5 mL/mmol) Boc₂O (1.2 equivalents) was added and then stirred at room temperature overnight. The reaction progress was monitored by TLC and LC-MS. When analyses indicated completion of the reaction, DCM was removed in vacuo and the crude product was purified by silica-gel or flash column chromatography on silica.

### General Procedure IIIa

Removal of the Tert-Butoxycarbonyl (Boc-) Group from Amine with HCl.

The N-Boc protected amine was treated with a 4 M solution of HCl (5 mL/mmol of starting material) in an appropriate organic solvent (e.g., AcOEt, 1,4-dioxane, MeOH, DCM) for the time necessary for complete consumption of the starting material (typically 30 minutes-2 hours). The volatiles were then removed in vacuo providing de-protected amine in the form of its hydrochloride salt. The crude product was usually purified by preparative reversed-phase column chromatography to give the corresponding product.

### General Procedure IIIb

Removal of the Tert-Butoxycarbonyl (Boc-) Group from Amine with TFA.

The N-Boc protected amine was treated with solution of TFA (6 equivalents) in DCM for the time necessary for complete consumption of the starting material (typically 30 minutes-2 hours). The volatiles were then removed in vacuo providing de-protected amine in the form of its TFA salt. The crude product was usually purified by preparative reversed-phase column chromatography to give the corresponding product.

### General Procedure IV

### The Suzuki Coupling.

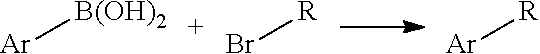

A palladium source (Pd(PPh₃)₄ or Pd(PPh₃)₂Cl₂ (0.05-0.1 equivalent)/PPh₃ (0.1-0.2 equivalent) or Pd(dppf)Cl₂ (0.05-0.1 equivalent) was dissolved in a combination of solvents such as dioxane (1-1.5 mL/mmol)/water (0.3 mL/mmol) and the mixture was degassed by bubbling with Ar. An appropriate halogen compound (1 equivalent) and K₂CO₃ (2-3 equivalents) were added and stirred for 5 minutes. Then to this mixture an organoboronic compound (1-2.5 equivalents) was added and the mixture was stirred vigorously at 90-110° C. (1-24 hours) under Ar. The reaction progress was monitored by TLC and LC-MS. After analytical control indicated completion of the reaction, the reaction was diluted with AcOEt (60 mL/mmol) and filtered through a Pad of the Celite, evaporated thoroughly and redissolved in AcOEt/H₂O. The layers were separated and the aqueous one was extracted with AcOEt (3×). The combined organic layers were washed with brine, dried over anhydrous Na₂SO₄, filtered and concentrated in vacuo. The crude product was purified by flash column chromatography on silica.

### General Procedure V

### Installation of the Appropriate R-Group on the Primary or Secondary Amine or Alcohol

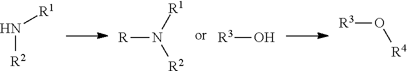

To a solution of the primary or secondary amine or alcohol in DCM (10 mL/mmol) or THE (4 mL/mmol), Et₃N (2-4 equivalents) or DIPEA (2 equivalents) followed by DMAP (0.1 equivalent, when it was necessary) were added and then appropriate carbamoyl chloride (2.5 equivalents) or appropriate isocyanate (1.1-2.5 equivalents) or acid chloride (1-2 equivalents) or appropriate anhydride (1.1-2 equivalents) was added at 0° C. or at room temperature. The reaction progress was monitored by TLC and LC-MS. When analyses indicated completion of the reaction, the mixture was quenched by addition of 4 M NaOH or 5% NaHCO₃ (for tosylation). Product was extracted with DCM (2 times). The combined organic solutions were dried over anhydrous Na₂SO₄, filtered and concentrated in vacuo and the crude product was purified by preparative reversed-phase column chromatography or by silica-gel column chromatography or by flash column chromatography on silica.

### General Procedure VI

### Synthesis of 4-chloropyrrolo[2,1-f][1,2,4]triazine-6-carboxylate

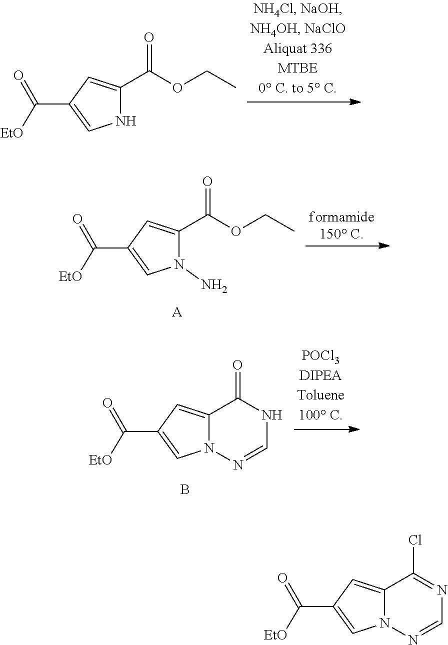

### Step 1.

### Synthesis of diethyl 1-amino-1H-pyrrole-2,4-dicarboxylate (A)

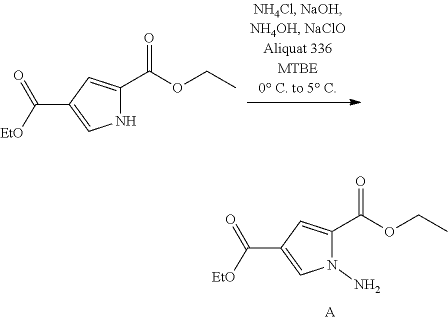

The cold (4° C.) 28% sodium hydroxide (610 g; 469 mL; 4270 mmol) was slowly poured to the suspension of 1H-pyrrole-2,4-dicarboxylic acid diethyl ester (36.1 g; 30.2 mL; 171 mmol), ammonium chloride (54.8 g; 35.8 mL; 1025 mmol) and aliquat 336 (2.07 g; 2.33 mL; 5.13 mmol) in MTBE (900 mL; 25 vol./wt) at 0° C. After 10 minutes 10% sodium hypochlorite (1145 g; 1044 mL; 1538 mmol) was added dropwise to an intensively stirred (300 rpm, overhead stirrer) reaction mixture in 45 minutes, maintaining temperature below 5° C. The mixture was intensively stirred at 5° C. for 120 minutes. Stirring was turned off and after 10 minutes phases were separated at 5° C. The organic phase was washed with 5% Na₂S₂O₃ solution (360 mL; 10 vol./wt) and 20% brine (180 mL; 5 vol./wt). The organic phase was evaporated in vacuo. The oily residue was evaporated with MTBE (100 mL) to remove residual water and to give the crude as opaque yellow oil (38.74 g; 100% yield). The crude was diluted with MTBE (100 mL; 2.5 vol./wt crude) and filtered through the silica gel pad (1.5 wt/wt crude), eluting with MTBE under reduced pressure. The eluate was collected, when yellow colour was observed. After evaporation and drying in vacuo the product was obtained as an orange oil, which crystallized overnight (34.99 g; 91% yield, purity (NMR)=c.a. 95%ₘₒₗ−contained 5%ₘₒₗ of substrate). The product A was stored in a fridge (3° C.).

¹H NMR (400 MHz, CDCl₃) δ 7.50-7.47 (m, 1H), 7.25-7.21 (m, 1H), 5.65 (s, 2H), 4.36-4.23 (m, 4H), 1.38-1.30 (m, 6H).

### Step 2.

### Synthesis of ethyl 4-oxo-3,4-dihydropyrrolo[2,1-f][1,2,4]triazine-6-carboxylate (B)

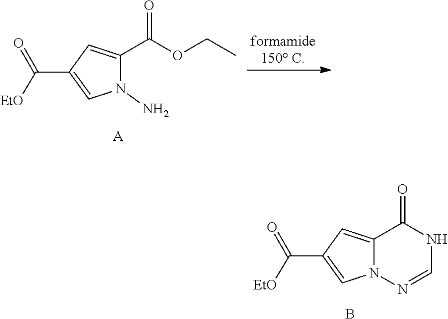

The solution of A (29.7 g; 131 mmol) in formamide (149 mL; 3711 mmol; 99.6%) was stirred at 150° C. under an Argon atmosphere. The progress of the reaction was monitored by TLC or HPLC. After completion of the reaction (225 minutes) a mixture was cooled to room temperature to give thick suspension. The suspension was diluted with water (150 mL). The precipitate was filtered off and then was washed with water (2×75 mL), MeCN (3×30 mL) and DCM (30 mL). The solid was dried in vacuo to give the crude B as a beige powder in 80% yield (21.65 g; 104 mmol).

¹H NMR (400 MHz, Methanol-d₄) δ 7.95 (d, J=1.8 Hz, 1H), 7.75 (s, 1H), 7.35 (d, J=1.8 Hz, 1H), 4.32 (q, J=7.1 Hz, 2H), 1.36 (t, J=7.1 Hz, 3H).

### Step 3.

### Synthesis of ethyl 4-chloropyrrolo[2,1-f][1,2,4]triazine-6-carboxylate

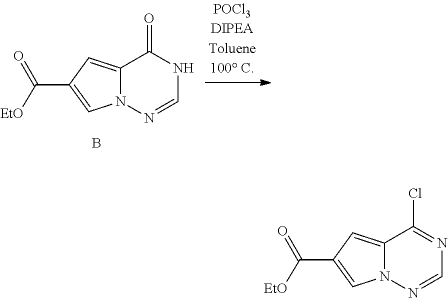

To the solution of B (21.6 g; 104 mmol) and N,N-diisopropylethylamine (14.8 g; 19.5 mL; 115 mmol) phosphorus (V) oxychloride (48.4 g; 29.4 mL; 313 mmol; 99%) was slowly added at room temperature under an Argon atmosphere. The mixture was heated to 100° C. and then left stirring overnight (22 hours). The postreaction mixture was cooled to 0° C. and neutralised with dropwise addition of cold (3° C.) 4 M NaOH solution (330 g; 287 mL; 1147 mmol) maintaining temperature below 20° C. The mixture was left stirring for 15 minutes and filtered through the Celite pad. The phases were separated. Aa aqueous phase was washed with AcOEt (110 mL; 5 vol./wt). The combined organic phases were washed with 20% brine (110 mL; 5 vol./wt) and evaporated in vacuo. The obtained brown oil was evaporated with new portion of toluene (50 mL) to remove residual water and give a crude as brown oil (53.3 g). The crude was diluted with toluene (70 mL) and filtered through the silica gel pad (1 wt/wt crude), eluting with 20% MTBE/toluene (c.a. 200 mL). The product was being collected only when blue fluorescence was visible under UV (365 nm) irradiation. After evaporation the residue was solvent swapped with DCM and dried in vacuo to give yellow solid (24.09 g). The residue was dissolved in DCM (25 mL; 1 vol./wt crude) and iPr₂O (75 mL; 3 vol./wt crude) was added and the mixture was condensed on rotary evaporator to c.a. 50 mL and the precipitate started to form. After 1 hour the precipitate was filtered off and washed with iPr₂O (25 mL). The solid was dried in vacuo to give a product as a pale yellow sand coloured powder in 84% yield (19.58 g; 87 mmol; purity (HPLC)=96.0%).

¹H NMR (400 MHz, CDCl₃) δ 8.29-8.26 (m, 2H), 7.41 (d, J=1.6 Hz, 1H), 4.40 (q, J=7.2 Hz, 2H), 1.41 (t, J=7.2 Hz, 3H).

### Exemplary Synthetic Procedures

### Example 1

### Synthesis of 3-((4-(5-chloro-3-methyl-2-(((S)-piperidin-3-yl)oxy)phenyl)pyrrolo[2,1-f][1,2,4]triazin-6-yl)methyl)-6,6-dimethyl-3-azabicyclo[3.1.0]hexane-2,4-dione hydrochloride (1)

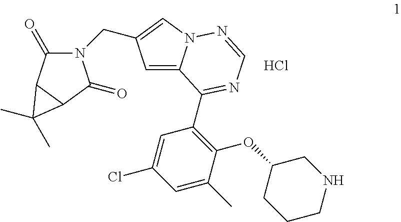 [SMILES: Cc1cc(Cl)cc(-c2ncnn3cc(CN4C(=O)C5C(C4=O)C5(C)C)cc23)c1O[C@H]1CCCNC1 ; Cl]

### Step 1.

### Synthesis of (4-chloropyrrolo[2,1-f][1,2,4]triazin-6-yl)methanol (1a)

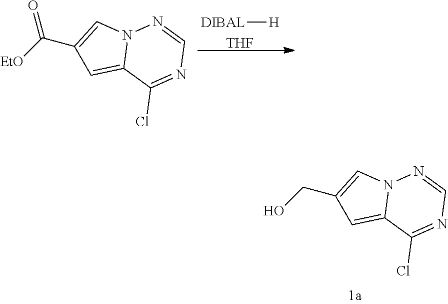

To a stirred solution of ethyl 4-chloropyrrolo[2,1-f][1,2,4]triazine-6-carboxylate (1.00 g; 4.43 mmol) in dry THF (60 mL) DIBAL-H (1 M in hexane; 22.16 mL; 22.16 mmol) was added at −78° C. Then the reaction mixture was allowed to warm to room temperature and stirred at this temperature for 3 hours. The reaction progress was monitored by LC-MS. Next the reaction mixture was cooled to 0° C. and quenched with a saturated solution of NH₄Cl (40 mL). Then filtered through a pad of Celite. An organic layer was dried over anhydrous Na₂SO₄, filtered and concentrated in vacuo and the crude product was purified by flash column chromatography on silica (hexane/AcOEt, 100:0 to 0:100, v/v, 30 minutes). Compound 1a was obtained as a white solid in 29% yield (235 mg: 1.28 mmol).

ESI-MS m/z for C₇H₇ClN₃O found 184.0/185.8 [M+H]⁺; Rₜ=0.58 min (Method A); ¹H NMR (700 MHz, CDCl₃) δ 8.23 (s, 1H), 7.94-7.91 (m, 1H), 7.01-6.98 (m, 1H), 4.89 (dt, J=5.7, 0.7 Hz, 2H), 1.83 (t, J=5.7 Hz, 1H).

### Step 2.

### Synthesis of 3-((4-chloropyrrolo[2,1-f][1,2,4]triazin-6-yl)methyl)-6,6-dimethyl-3-azabicyclo[3.1.0]hexane-2,4-dione (1b)

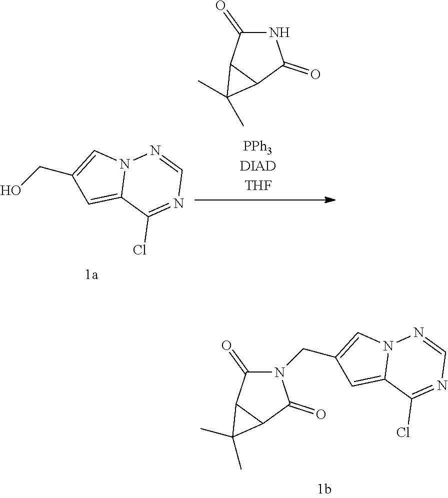

To a cooled to −10° C. solution of 1a (50 mg; 0.27 mmol), 6,6-dimethyl-3-azabicyclo[3.1.0]hexane-2,4-dione (38 mg; 0.27 mmol) and PPh₃ (85 mg; 0.32 mmol) in THE (5 mL) DIAD (69 μL; 0.35 mmol) was added. The resulting mixture was stirred at room temperature for 1 hour. Then the reaction mixture was concentrated in vacuo and the residue was purified by flash column chromatography on silica (hexane/AcOEt, 100:0 to 0:100, v/v, 25 minutes). Compound 1b was obtained in 70% yield (59 mg; 0.19 mmol).

ESI-MS m/z for C₁₄H₁₄ClN₄O₂ found 304.9/306.6 [M+H]⁺; Rₜ=1.18 min (Method A)

### Step 3.

### Synthesis of tert-butyl (S)-3-(2-bromo-4-chloro-6-methylphenoxy)piperidine-1-carboxylate (1c)

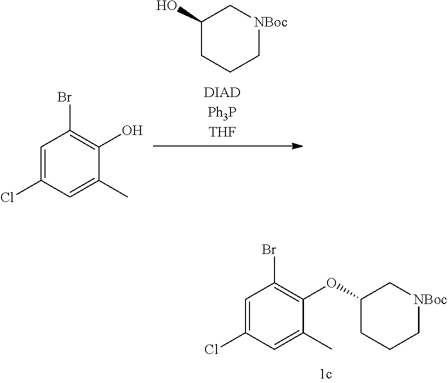

To a cooled to −10° C. solution of 2-bromo-4-chloro-6-methylphenol (9.9 g; 47.7 mmol), tert-butyl (R)-3-hydroxypiperidine-1-carboxylate (9.9 g; 49.2 mmol) and PPh₃ (14 g; 53.6 mmol) in THE (250 ml) DIAD (10.5 mL; 53.6 mmol) was slowly added. The resulting mixture was stirred at room temperature overnight. The formed solid was filtered and washed with hexane (3×), then purified by silica-gel column chromatography (hexane/AcOEt, 100:1 to 15:1, v/v). Compound 1c was obtained in 46% yield (8.9 g; 22.1 mmol).

ESI-MS m/z for C₁₇H₂₃BrClNO₃Na found 427.8/429.8 [M+Na]⁺; Rₜ=2.08 min (Method A); ¹H NMR (700 MHz, CDCl₃) δ 7.45-7.35 (m, 1H), 7.17-7.05 (m, 1H), 4.16-4.01 (m, 2H), 3.17-2.89 (m, 2H), 2.30 (s, 3H), 2.17-2.04 (m, 1H), 1.87-1.80 (m, 1H), 1.79-1.72 (m, 1H), 1.52-1.48 (m, 1H), 1.45 (s, 9H), 1.33-1.26 (m, 1H).

### Step 4.

### Synthesis of tert-butyl (S)-3-(4-chloro-2-methyl-6-(4,4,5,5-tetramethyl-1,3,2-dioxaborolan-2-yl)phenoxy)piperidine-1-carboxylate (1d)

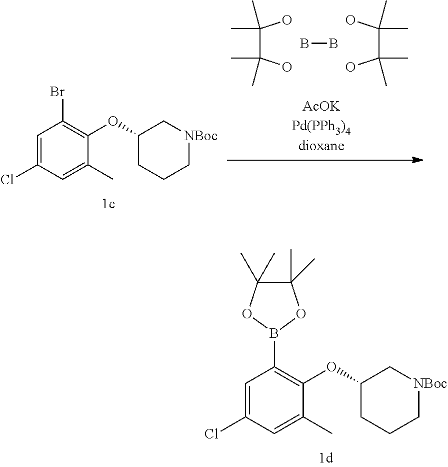

To the solution of 1c (5.00 g; 12.35 mmol) in dry dioxane (190 mL) bis(pinacolato)diboron (6.27 g; 24.70 mmol) and AcOK (3.60 g; 37.05 mmol) were added. The reaction mixture was intensively flushed with Ar. Then to this mixture Pd(PPh₃)₄ (0.71 g; 0.62 mmol) was added in one portion and the reaction mixture was flushed with Ar. The mixture was stirred overnight at 100° C. in a sealed tube. The reaction progress was monitored by LC-MS. Then another portion of Pd(PPh₃)₄ (0.30 g; 0.26 mmol), AcOK (2.42 g; 24.91 mmol) and bis(pinacolato)diboron (6.27 g; 24.70 mmol) was added and this mixture was stirred at 110° C. for 2 days. When analysis indicated completion of the reaction, dioxane was removed and the residue was dissolved in AcOEt/water and filtered through a pad of the Celite. The filtrate was separated and the aqueous layer was extracted with AcOEt. The combined organic layers were dried over anhydrous MgSO₄, filtered and concentrated in vacuo. The crude product was then purified by silica-gel column chromatography (hexane/AcOEt, 100:1 to 15:1, v/v). Compound 1d was obtained in 90% yield (5.00 g; 11.08 mmol).

ESI-MS m/z for C₂₃H₃₅BClNO₅Na found 474.0/476.0 [M+Na]⁺; Rₜ=2.24 min (Method A)

### Step 5.

### Synthesis of tert-butyl (3S)-3-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenoxy)piperidine-1-carboxylate (1e)

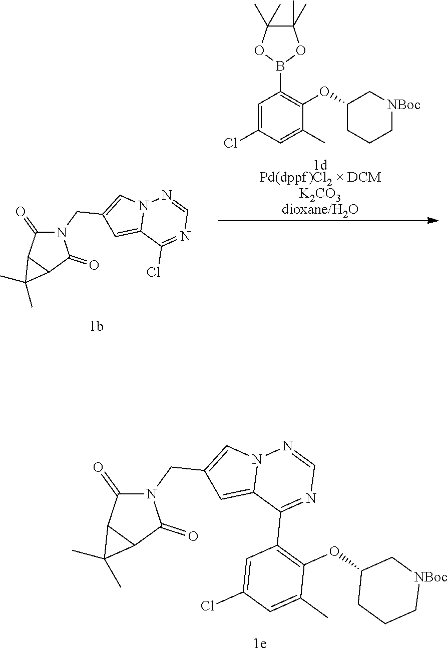

The title compound (1e) was obtained from 1b (50 mg; 0.164 mmol) and from 1d (74 mg; 0.164 mmol) according to the General Procedure IV and after standard work-up the crude product was taken to the next step.

ESI-MS m/z for C₃₁H₃₇ClN₅O₅ found 594.4/596.3 [M+H]⁺; Rₜ=1.90 min (Method A)

### Step 6.

### Synthesis of 3-((4-(5-chloro-3-methyl-2-(((S)-piperidin-3-yl)oxy)phenyl)pyrrolo[2,1-f][1,2,4]triazin-6-yl)methyl)-6,6-dimethyl-3-azabicyclo[3.1.0]hexane-2,4-dione hydrochloride

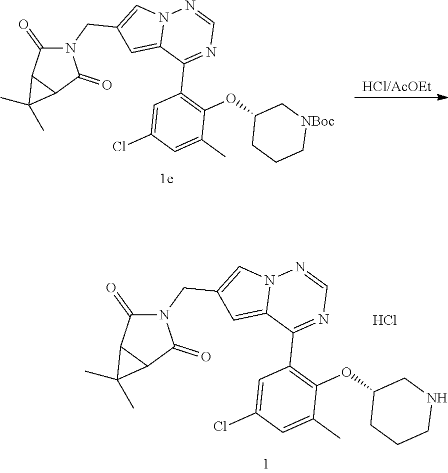 [SMILES: Cc1cc(Cl)cc(-c2ncnn3cc(CN4C(=O)C5C(C4=O)C5(C)C)cc23)c1O[C@H]1CCCN(C(=O)OC(C)(C)C)C1 ; "HCl/AcOEt" ; Cc1cc(Cl)cc(-c2ncnn3cc(CN4C(=O)C5C(C4=O)C5(C)C)cc23)c1O[C@H]1CCCNC1 ; Cl]

The title compound (1) was obtained as a hydrochloride salt from 1e (the crude product) according to the General Procedure IIIa in 35% yield (per two steps) (30 mg; 0.057 mmol). The crude product was purified by preparative reversed-phase column chromatography (C-18, water+0.3‰ HCl (36%)/MeCN, 98:2 to 30:70, 30 min, 20 mL/min, Rₜ=17.5 min).

ESI-MS m/z for C₂₆H₂₉ClN₅O₃ found 494.4/496.4 [M+H]⁺; Rₜ=1.00 min (Method A); ¹H NMR (700 MHz, Methanol-d₄) δ 8.58 (s, 1H), 8.14-8.08 (m, 1H), 7.61-7.55 (m, 1H), 7.49-7.46 (m, 1H), 6.89-6.87 (m, 1H), 4.72-4.61 (m, 2H), 4.05-3.98 (m, 1H), 3.15-3.09 (m, 1H), 3.09-3.03 (m, 2H), 3.00-2.94 (m, 1H), 2.49 (s, 2H), 2.47 (s, 3H), 1.76-1.70 (m, 1H), 1.69-1.64 (m, 1H), 1.53-1.43 (m, 2H), 1.26 (s, 3H), 1.09 (s, 3H).

### Example 2

### Synthesis of 3-((4-(5-chloro-3-methyl-2-(((R)-morpholin-2-yl)methyl)phenyl)pyrrolo[2,1-f][1,2,4]triazin-6-yl)methyl)-6,6-dimethyl-3-azabicyclo[3.1.0]hexane-2,4-dione hydrochloride (2)

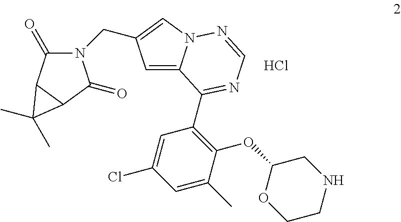 [SMILES: Cc1cc(Cl)cc(-c2ncnn3cc(CN4C(=O)C5C(C4=O)C5(C)C)cc23)c1O[C@@H]1CNCCO1 ; Cl]

### Step 1.

### Synthesis of tert-butyl (R)-2-(4-chloro-2-methyl-6-(4,4,5,5-tetramethyl-1,3,2-dioxaborolan-2-yl)benzyl)morpholine-4-carboxylate (2a)

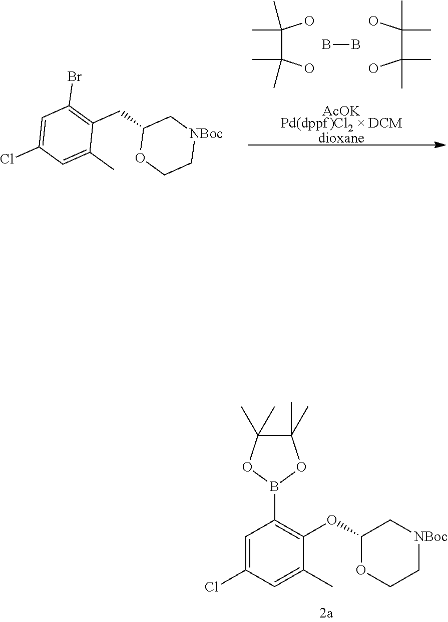

In a Schlenk flask was placed tert-butyl (R)-2-(2-bromo-4-chloro-6-methylbenzyl)morpholine-4-carboxylate (the synthesis of this compound was described in J. Med. Chem. 2020, 63, 5398-5420)(100 mg; 0.248 mmol), bis(pinacolato)diboron (126 mg; 0.500 mmol), AcOK (73 mg; 0.744 mmol) and Pd(dppf)Cl₂×DCM (20 mg; 0.025 mmol) and dioxane (2 mL). The reaction mixture was intensively flushed with Ar. The mixture was capped and stirred at 90° C. overnight. The reaction progress was monitored by LC-MS. When analysis indicated completion of the reaction, the reaction mixture was concentrated in vacuo. The residue was then purified by flash column chromatography on silica (hexane/AcOEt, 100:0 to 40:60, v/v, 27 min). Compound 2a was obtained in 98% yield (110 mg; 0.244 mmol).

ESI-MS m/z for C₁₈H₂₈BClNO₃ found 352.0/354.0 [M+H-Boc]⁺; Rₜ=2.30 min (Method A); ¹H NMR (700 MHz, CDCl₃) δ 7.64-7.61 (m, 1H), 7.24-7.22 (m, 1H), 4.08-3.92 (m, 1H), 3.84-3.74 (m, 2H), 3.48-3.40 (m, 1H), 3.37-3.29 (m, 1H), 3.15-3.08 (m, 1H), 3.07-2.99 (m, 1H), 2.95-2.88 (m, 1H), 2.74-2.65 (m, 1H), 2.36 (s, 3H), 1.47 (s, 9H), 1.38 (s, 6H), 1.36 (s, 6H).

### Step 2.

### Synthesis of tert-butyl (2R)-2-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylbenzyl)morpholine-4-carboxylate (2b)

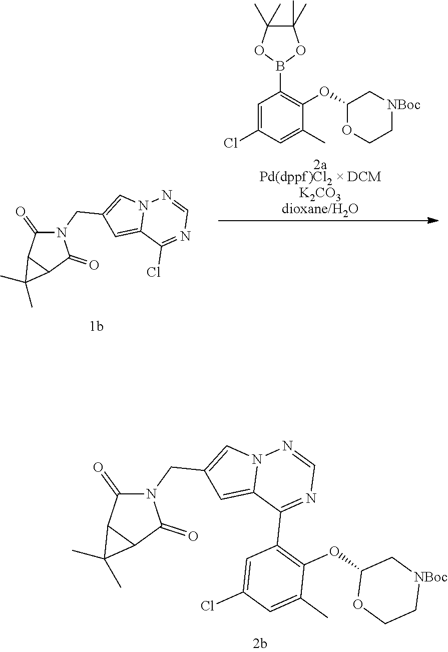

The title compound (2b) was obtained from 1b (80 mg; 0.260 mmol) and from 2a (118 mg; 0.260 mmol) according to the General Procedure IV in 33% yield (51 mg; 0.086 mmol). The crude product was purified by flash column chromatography on silica (hexane/AcOEt, 100:0 to 20:80, v/v, 28 min).

ESI-MS m/z for C₃₁H₃₇ClN₅O₅ found 594.1/596.2 [M+H]⁺; Rₜ=1.88 min (Method A)

### Step 3.

### Synthesis of 3-((4-(5-chloro-3-methyl-2-(((R)-morpholin-2-yl)methyl)phenyl)pyrrolo[2,1-f][1,2,4]triazin-6-yl)methyl)-6,6-dimethyl-3-azabicyclo[3.1.0]hexane-2,4-dione hydrochloride (2)

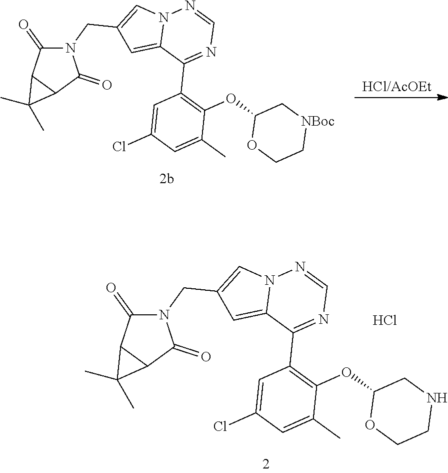 [SMILES: Cc1cc(Cl)cc(-c2ncnn3cc(CN4C(=O)C5C(C4=O)C5(C)C)cc23)c1O[C@@H]1CN(C(=O)OC(C)(C)C)CCO1 ; "HCl/AcOEt" ; Cc1cc(Cl)cc(-c2ncnn3cc(CN4C(=O)C5C(C4=O)C5(C)C)cc23)c1O[C@@H]1CNCCO1 ; Cl]

The title compound (2) was obtained as a hydrochloride salt from 2b (49 mg; 0.082 mmol) according to the General Procedure IIIa in 57% yield (25 mg; 0.047 mmol). The crude product was purified by preparative reversed-phase column chromatography (C-18, water+0.3‰ HCl (36%)/MeCN, 98:2 to 30:70, 30 min, 20 mL/min, Rₜ=17.5 min).

ESI-MS m/z for C₂₆H₂₉ClN₅O₃ found 494.4/496.4 [M+H]⁺; Rₜ=1.02 min (Method A); ¹H NMR (700 MHz, Methanol-d₄) δ 8.54 (s, 1H), 8.10-8.04 (m, 1H), 7.52-7.48 (m, 1H), 7.38-7.34 (m, 1H), 6.68-6.64 (m, 1H), 4.70-4.66 (m, 2H), 3.83-3.74 (m, 2H), 3.54-3.46 (m, 1H), 3.20-3.09 (m, 2H), 3.03-2.88 (m, 3H), 2.79-2.73 (m, 1H), 2.51 (s, 3H), 2.48 (s, 2H), 1.25 (s, 3H), 1.08 (s, 3H).

### Example 3

### Synthesis of 3-((4-(5-chloro-3-methyl-2-(((3R,6R)-6-methylpiperidin-3-yl)oxy)phenyl)pyrrolo[2,1-f][1,2,4]triazin-6-yl)methyl)-6,6-dimethyl-3-azabicyclo[3.1.0]hexane-2,4-dione hydrochloride (3)

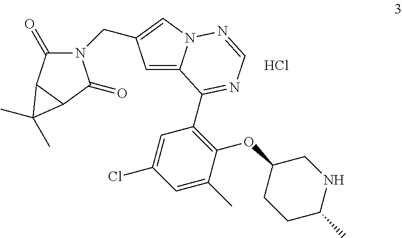

The title compound (3) was obtained as a hydrochloride salt as a single isomer in 5% overall yield in a similar way to Example 1 with the exception that, in the third step of the synthesis, tert-butyl (2R,5S)-5-hydroxy-2-methylpiperidine-1-carboxylate was used instead of tert-butyl (R)-3-hydroxypiperidine-1-carboxylate. The crude product was purified by preparative reversed-phase column chromatography (C-18, water+0.3‰ HCl (36%)/MeCN, 98:2 to 30:70, 30 min, 20 mL/min, Rₜ=17.8 min).

ESI-MS m/z for C₂₇H₃₁ClN₅O₃ found 508.4/510.4 [M+H]⁺; Rₜ=1.04 min (Method A); ¹H NMR (700 MHz, Methanol-d₄) δ 8.58 (s, 1H), 8.13-8.10 (m, 1H), 7.59-7.57 (m, 1H), 7.52-7.48 (m, 1H), 6.89-6.85 (m, 1H), 4.70 (s, 2H), 3.89-3.79 (m, 1H), 3.20-3.14 (m, 1H), 3.14-3.07 (m, 1H), 2.82-2.75 (m, 1H), 2.49 (s, 2H), 2.45 (s, 3H), 1.88-1.81 (m, 1H), 1.82-1.75 (m, 1H), 1.44-1.35 (m, 1H), 1.26 (s, 3H), 1.22 (d, J=6.5 Hz, 3H), 1.10 (s, 3H).

### Example 4

### Synthesis of 3-((4-(5-chloro-3-methyl-2-(piperidin-4-yloxy)phenyl)pyrrolo[2,1-f][1,2,4]triazin-6-yl)methyl)-6,6-dimethyl-3-azabicyclo[3.1.0]hexane-2,4-dione hydrochloride (4)

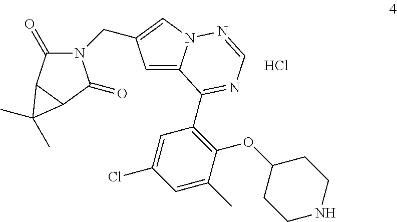 [SMILES: Cc1cc(Cl)cc(-c2ncnn3cc(CN4C(=O)C5C(C4=O)C5(C)C)cc23)c1OC1CCNCC1 ; Cl]

The title compound (4) was obtained as a hydrochloride salt as a single isomer in 1% overall yield in a similar way to Example 1 with the exception that, in the third step of the synthesis, tert-butyl 4-hydroxypiperidine-1-carboxylate was used instead of tert-butyl (R)-3-hydroxypiperidine-1-carboxylate and in the sixth step of the synthesis, the General Procedure IIIb was used instead of the General Procedure IIIa. The crude product was purified twice by preparative reversed-phase column chromatography (first: C-18, water+0.3‰ HCl (36%)/MeCN, 95:5 to 50:50, 30 min, 16 mL/min, Rₜ=23.4 min, second: column: Cosmosil Cholester 20×250 mm, water+0.3‰ HCl (36%)/MeCN, 99:1 to 50:50, 30 min, 20 mL/min, Rₜ=21.7 min).

ESI-MS m/z for C₂₆H₂₉ClN₅O₃ found 494.3/496.3 [M+H]⁺; Rₜ=1.06 min (Method A); ¹H NMR (700 MHz, MeCN-d₃) δ 8.56 (s, 1H), 8.10-8.04 (m, 1H), 7.57-7.52 (m, 1H), 7.50-7.43 (m, 1H), 6.90 (s, 1H), 4.64 (s, 2H), 3.98-3.90 (m, 1H), 2.89-2.83 (m, 2H), 2.79-2.72 (m, 2H), 2.42 (s, 2H), 2.41 (s, 3H), 1.90-1.80 (m, 2H), 1.70-1.63 (m, 2H), 1.23 (s, 3H), 1.08 (s, 3H).

### Example 5

### Synthesis of 6,6-dimethyl-3-((4-(1-(pyrrolidin-3-yl)-5-(trifluoromethyl)-1H-indol-7-yl)pyrrolo[2,1-f][1,2,4]triazin-6-yl)methyl)-3-azabicyclo[3.1.0]hexane-2,4-dione 2,2,2-trifluoroacetate (5)

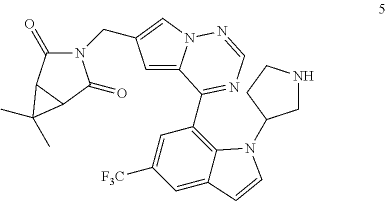 [SMILES: CC1(C)C2C(=O)N(Cc3cc4c(-c5cc(C(F)(F)F)cc6ccn(C7CCNC7)c56)ncnn4c3)C(=O)C21]

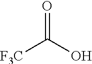

### Step 1.

### Synthesis of tert-butyl 3-(7-bromo-5-(trifluoromethyl)-1H-indol-1-yl)pyrrolidine-1-carboxylate (5a)

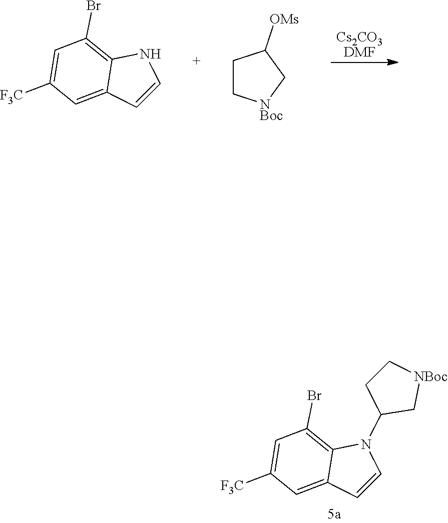

The solution of 7-bromo-5-(trifluoromethyl)-1H-indole (32 mg; 0.122 mmol), tert-butyl 3-((methylsulfonyl)oxy)pyrrolidine-1-carboxylate (36 mg; 0.136 mmol) and Cs₂CO₃ (89 mg; 0.272 mmol) in DMF (1 mL) was stirred under argon atmosphere at room temperature for 4 hours and then at 80° C. overnight. Then another portion of tert-butyl 3-((methylsulfonyl)oxy)pyrrolidine-1-carboxylate (36 mg; 0.136 mmol) was added and a whole was stirred at 80° C. overnight. The reaction progress was monitored by LC-MS. When analysis indicated completion of the reaction, a whole was concentrated in vacuo. The crude product was then purified by flash column chromatography on silica (hexane/AcOEt, 100:0 to 60:40, v/v, 25 min). Compound 5a was obtained in 59% yield (31 mg; 0.072 mmol).

ESI-MS m/z for C₁₄H₁₃BrF₃N₂O₂ found 377.0/379.0 [M+H-tBu]⁺; Rₜ=2.06 min (Method A)

### Step 2.

### Synthesis of tert-butyl 3-(7-(4,4,5,5-tetramethyl-1,3,2-dioxaborolan-2-yl)-5-(trifluoromethyl)-1H-indol-1-yl)pyrrolidine-1-carboxylate (5b)

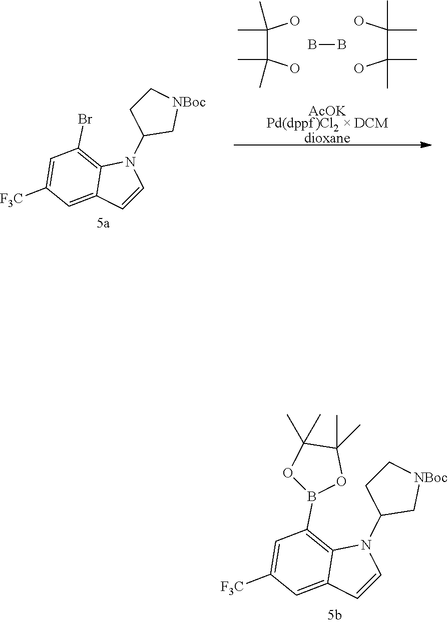

In a Schlenk flask was placed the compound 5a (33 mg; 0.076 mmol), bis(pinacolato)diboron (39 mg; 0.152 mmol), AcOK (22 mg; 0.229 mmol) and Pd(dppf)Cl₂×DCM (6 mg; 0.008 mmol) and dioxane (0.6 mL). The reaction mixture was intensively flushed with Ar. The mixture was capped and stirred at 90° C. overnight. The reaction progress was monitored by LC-MS. When analysis indicated completion of the reaction, the reaction mixture was concentrated in vacuo. The residue was then purified by flash column chromatography on silica (hexane/AcOEt, 100:0 to 70:30, v/v, 27 min). Compound 5b was obtained in 71% yield (26 mg; 0.054 mmol).

ESI-MS m/z for C₂₀H₂₅BF₃N₂O₄ found 425.0 [M+H-tBu]⁺; Rₜ=2.23 min (Method A)

### Step 3.

### Synthesis of tert-butyl 3-(7-(6-((6,6-dimethyl-2,4-dioxo-3-azabicyclo[3.1.0]hexan-3-yl)methyl)pyrrolo[2,1-f][1,2,4]triazin-4-yl)-5-(trifluoromethyl)-1H-indol-1-yl)pyrrolidine-1-carboxylate (5c)

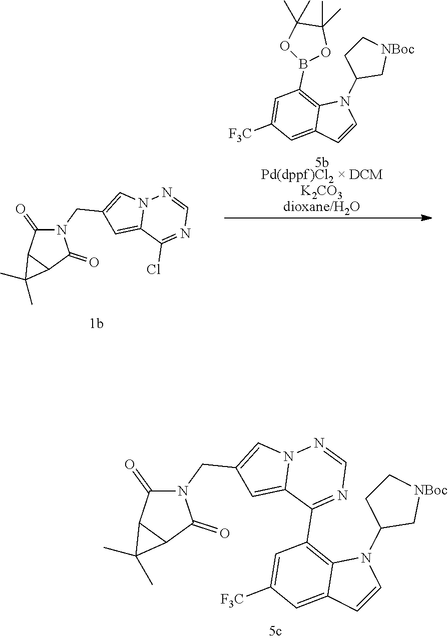

The title compound (5c) was obtained from 1b (33 mg; 0.108 mmol) and from 5b (26 mg; 0.054 mmol) according to the General Procedure IV. The crude product was used to the next step without any additional purification.

ESI-MS m/z for C₃₂H₃₄F₃N₆O₄ found 623.0 [M+H]⁺; Rₜ=1.89 min (Method A)

### Step 4.

### Synthesis of 6,6-dimethyl-3-((4-(1-(pyrrolidin-3-yl)-5-(trifluoromethyl)-1H-indol-7-yl)pyrrolo[2,1-f][1,2,4]triazin-6-yl)methyl)-3-azabicyclo[3.1.0]hexane-2,4-dione 2,2,2-trifluoroacetate (5)

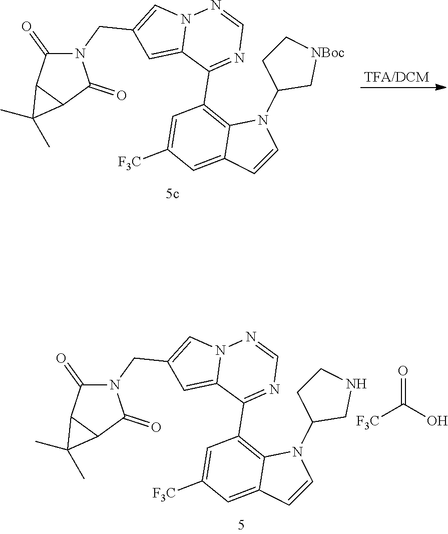

The title compound (5) was obtained as a TFA salt from 5c (the crude product) according to the General Procedure IIIb in 11% yield (per two steps)(4 mg; 0.006 mmol). The crude product was purified by preparative reversed-phase column chromatography (C-18, water+0.4‰ TFA/MeCN, 99:1 to 40:60, 30 min, 20 mL/min, Rₜ=23.7 min).

ESI-MS m/z for C₂₇H₂₆F₃N₆O₂ found 523.5 [M+H]⁺; Rₜ=1.10 min (Method A); ¹H NMR (700 MHz, MeCN-d₃) δ 8.56 (s, 1H), 8.21-8.18 (m, 1H), 8.06-8.02 (m, 1H), 7.66-7.59 (m, 2H), 6.98-6.93 (m, 1H), 6.65-6.61 (m, 1H), 4.98-4.91 (m, 1H), 4.63 (s, 2H), 3.50-3.42 (m, 2H), 3.38-3.30 (m, 1H), 3.21-3.15 (m, 1H), 2.41 (s, 2H), 2.27-2.19 (m, 2H), 1.20 (s, 3H), 1.02 (s, 3H).

### Example 6

### Synthesis of 3-((4-(5-chloro-3-methyl-2-(((S)-2-methylpiperazin-1-yl)methyl)phenyl)pyrrolo[2,1-f][1,2,4]triazin-6-yl)methyl)-6,6-dimethyl-3-azabicyclo[3.1.0]hexane-2,4-dione hydrochloride (6)

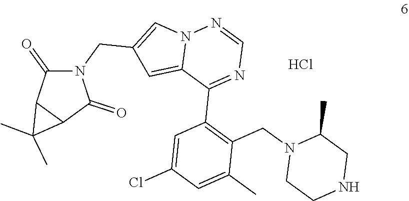 [SMILES: Cc1cc(Cl)cc(-c2ncnn3cc(CN4C(=O)C5C(C4=O)C5(C)C)cc23)c1CN1CCNC[C@@H]1C ; Cl]

### Step 1.

### Synthesis of (4-chloro-2-iodo-6-methylphenyl)methanol (6a)

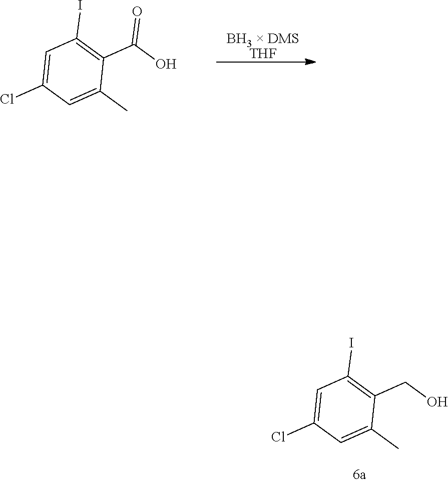

To a cooled to 0° C. solution of 4-chloro-2-iodo-6-methylbenzoic acid (1.33 g; 4.48 mmol) in dry THE (13 mL) BH3×DMS (0.85 mL; 8.97 mmol) was added dropwise under an Argon atmosphere. Then, this mixture was stirred at 65° C. overnight. Then the reaction was quenched with MeOH (10 mL) at 0° C. The mixture was concentrated in vacuo and the residue was purified by flash column chromatography on silica (hexane/AcOEt, 100:0 to 0:100, v/v, 27 min, 20 mL/min). Compound 6a was obtained in 77% yield (966 mg; 3.43 mmol).

¹H NMR (700 MHz, CDCl₃) δ 7.75-7.68 (m, 1H), 7.23-7.18 (m, 1H), 4.83 (s, 2H), 2.51 (s, 3H).

### Step 2.

### Synthesis of 2-(bromomethyl)-5-chloro-1-iodo-3-methylbenzene (6b)

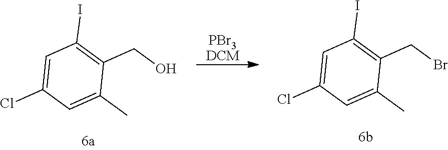

To a cooled to 0° C. solution of 6a (870 mg; 3.086 mmol) in dry DCM (15 mL) PBr₃ (580 μL; 6.170 mmol) was slowly added. The resulting mixture was allowed to warm to room temperature and stirred for 3 hours. The reaction progress was monitored by LC-MS. When analysis indicated completion of the reaction, the reaction mixture was cooled to 0° C. and quenched with saturated solution of NaHCO₃. The layers were separated and an aqueous one was then extracted with DCM (2×20 mL). The combined organic layers were washed with brine (1×30 mL), dried over anhydrous Na₂SO₄, filtered and concentrated in vacuo. Compound 6b was obtained in 70% yield (748 mg; 2.175 mmol).

ESI-MS m/z for C₈H₈BrClI found 344.6/346.4 [M+H]⁺; Rₜ=1.88 min (Method A); ¹H NMR (700 MHz, CDCl₃) δ 7.75-7.72 (m, 1H), 7.21-7.18 (m, 1H), 4.67 (s, 2H), 2.52-2.47 (m, 3H).

### Step 3.

### Synthesis of tert-butyl (S)-4-(4-chloro-2-iodo-6-methylbenzyl)-3-methylpiperazine-1-carboxylate (6c)

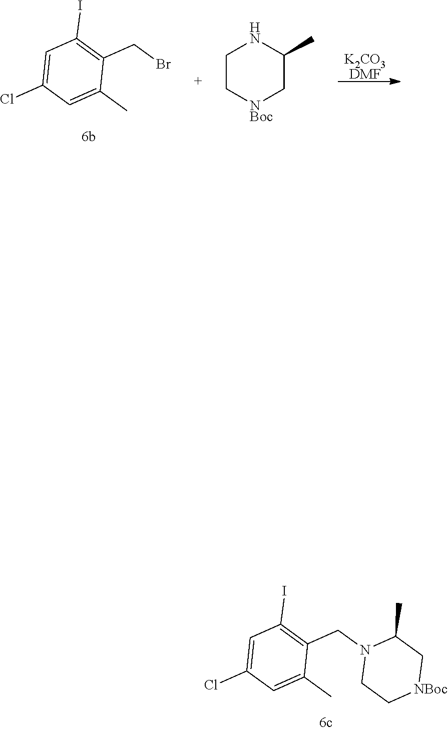

Ta a stirred solution of 6b (70 mg; 0.203 mmol) and tert-butyl (S)-3-methylpiperazine-1-carboxylate (41 mg; 0.203 mmol) in DMF (1 mL) K₂CO₃ (42 mg; 0.304 mmol) was added and was stirred under an Argon atmosphere at 50° C. for 4 hours. The reaction progress was monitored by LC-MS. When analysis indicated completion of the reaction, the reaction mixture wad cooled to room temperature and then diluted with AcOEt (30 mL), washed with water (10 mL), 1 M KHSO₄ (10 mL) and brine (10 mL), dried over anhydrous Na₂SO₄, filtered and concentrated in vacuo. Compound 6c was obtained in 99% yield (94 mg; 0.202 mmol).

ESI-MS m/z for C18H₂₇ClIN₂O₂ found 465.2/467.0 [M+H]⁺; Rₜ=1.76 min (Method A)

### Step 4.

### Synthesis of (S)-(2-((4-(tert-butoxycarbonyl)-2-methylpiperazin-1-yl)methyl)-5-chloro-3-methylphenyl)boronic acid (6d)

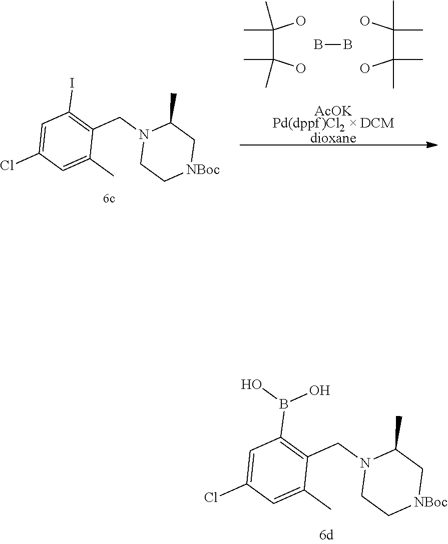

In a Schlenk flask was placed the compound 6c (90 mg; 0.194 mmol), bis(pinacolato)diboron (98 mg; 0.387 mmol), AcOK (57 mg; 0.582 mmol) and Pd(dppf)Cl₂×DCM (16 mg; 0.019 mmol) and dioxane (1.5 mL). The reaction mixture was intensively flushed with Ar. The mixture was capped and stirred at 90° C. overnight. The reaction progress was monitored by LC-MS. When analysis indicated completion of the reaction, the reaction mixture was concentrated in vacuo. The crude product was used to the next step without any additional purification.

ESI-MS m/z for C₁₈H₂₉BClN₂O₄ found 382.9/385.0 [M+H]⁺; Rₜ=1.16 min (Method A)

### Step 5.

### Synthesis of tert-butyl (3S)-4-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylbenzyl)-3-methylpiperazine-1-carboxylate (6e)

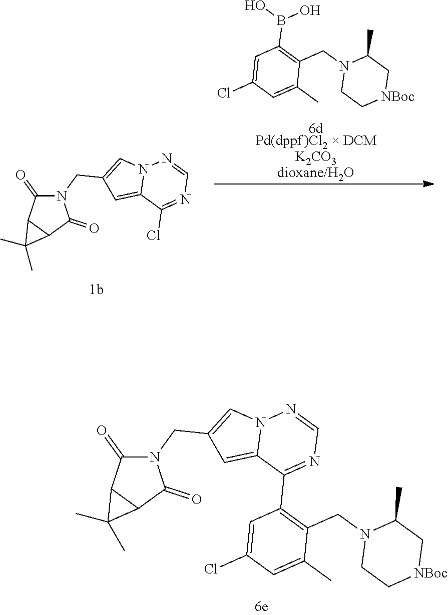

The title compound (6e) was obtained from 1b (59 mg; 0.194 mmol) and from 6d (the crude product) according to the General Procedure IV. The crude product was purified by flash column chromatography on silica (hexane/AcOEt, 100:0 to 0:100, v/v, 22 min, 9 mL/min) and then was used to the next step without any additional purification.

ESI-MS m/z for C₃₂H₄₀ClN₆O₄ found 607.5/609.2 [M+H]⁺; Rₜ=1.34 min (Method A)

### Step 6.

### Synthesis of 3-((4-(5-chloro-3-methyl-2-(((S)-2-methylpiperazin-1-yl)methyl)phenyl)pyrrolo[2,1-f][1,2,4]triazin-6-yl)methyl)-6,6-dimethyl-3-azabicyclo[3.1.0]hexane-2,4-dione hydrochloride (6)

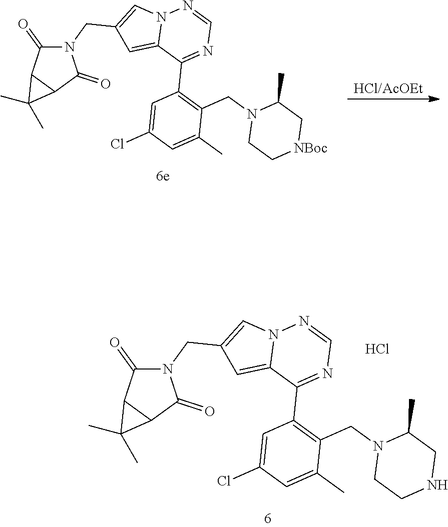 [SMILES: Cc1cc(Cl)cc(-c2ncnn3cc(CN4C(=O)C5C(C4=O)C5(C)C)cc23)c1CN1CCN(C(=O)OC(C)(C)C)C[C@@H]1C ; "HCl/AcOEt" ; Cc1cc(Cl)cc(-c2ncnn3cc(CN4C(=O)C5C(C4=O)C5(C)C)cc23)c1CN1CCNC[C@@H]1C ; Cl]

The title compound (6) was obtained as a hydrochloride salt from 6e (the crude product) according to the General Procedure IIIa in 2% yield (per three steps)(2 mg; 0.004 mmol). The crude product was purified by preparative reversed-phase column chromatography (column: Cosmosil Cholester 20×250 mm, water+0.3‰ HCl (36%)/MeCN, 95:5 to 20:80, 30 min, 20 mL/min, Rₜ=14.4 min).

ESI-MS m/z for C₂₇H₃₂ClN₆O₂ found 507.4/509.4 [M+H]⁺; Rₜ=1.04 min (Method A); ¹H NMR (700 MHz, Methanol-d₄) δ 8.64 (s, 1H), 8.16-8.11 (m, 1H), 7.66-7.59 (m, 2H), 6.83 (s, 1H), 4.74-4.66 (m, 2H), 4.53-4.47 (m, 1H), 4.00-3.86 (m, 1H), 3.31-3.27 (m, 1H), 3.27-3.19 (m, 2H), 3.04-2.92 (m, 2H), 2.91-2.77 (m, 2H), 2.59 (s, 3H), 2.49 (s, 2H), 1.26 (s, 3H), 1.13 (d, J=6.5 Hz, 3H), 1.11 (s, 3H).

### Example 7

### Synthesis of (2S,4S)-4-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenoxy)pyrrolidine-2-carboxylic acid hydrochloride (7)

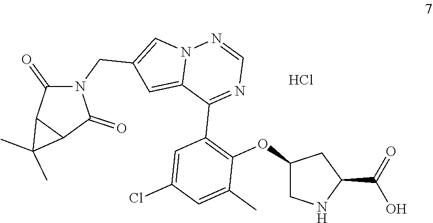 [SMILES: Cc1cc(Cl)cc(-c2ncnn3cc(CN4C(=O)C5C(C4=O)C5(C)C)cc23)c1O[C@@H]1CN[C@H](C(=O)O)C1 ; Cl]

The title compound (7) in hydrochloride form was obtained as a single isomer in 4% overall yield in a similar way to Example 1 with the exception that, in the third step of the synthesis, di-tert-butyl (2S,4R)-4-hydroxypyrrolidine-1,2-dicarboxylate was used instead of tert-butyl (R)-3-hydroxypiperidine-1-carboxylate. The crude product was purified by preparative reversed-phase column chromatography (C-18, water+0.3‰ HCl (36%)/MeCN, 95:5 to 20:80, 30 min, 20 mL/min, Rₜ=16.8 min).

ESI-MS m/z for C₂₆H₂₇ClN₅O₅ found 524.0/526.0 [M+H]⁺; Rₜ=1.06 min (Method A); ¹H NMR (700 MHz, Methanol-d₄) δ 8.58 (s, 1H), 8.13-8.07 (m, 1H), 7.57-7.55 (m, 1H), 7.46-7.42 (m, 1H), 6.82-6.78 (m, 1H), 4.74-4.64 (m, 2H), 4.61-4.53 (m, 1H), 4.46-4.38 (m, 1H), 3.44-3.37 (m, 1H), 3.30-3.22 (m, 1H), 2.53-2.46 (m, 2H), 2.43 (s, 3H), 2.42-2.35 (m, 1H), 2.20-2.15 (m, 1H), 1.25 (s, 3H), 1.08 (s, 3H).

### Example 8

### Synthesis of 3-((4-(5-chloro-2-(3,3-difluoroazetidine-1-carbonyl)-3-methylphenyl)pyrrolo[2,1-f][1,2,4]triazin-6-yl)methyl)-6,6-dimethyl-3-azabicyclo[3.1.0]hexane-2,4-dione hydrochloride (8)

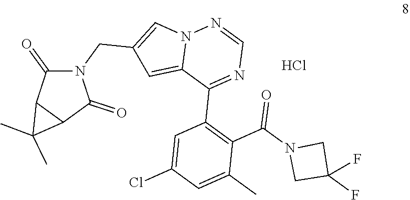 [SMILES: Cc1cc(Cl)cc(-c2ncnn3cc(CN4C(=O)C5C(C4=O)C5(C)C)cc23)c1C(=O)N1CC(F)(F)C1 ; Cl]

### Step 1.

### Synthesis of (4-chloro-2-iodo-6-methylphenyl)(3,3-difluoroazetidin-1-yl)methanone (8a)

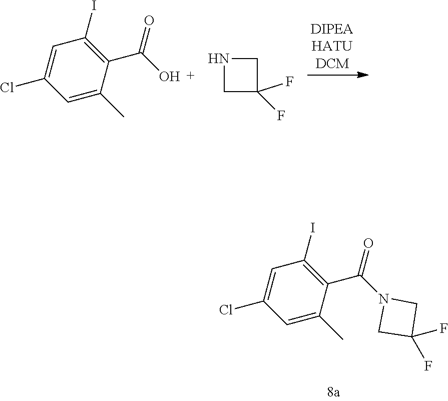 [SMILES: Cc1cc(Cl)cc(I)c1C(=O)O ; FC1(F)CNC1 ; Cc1cc(Cl)cc(I)c1C(=O)N1CC(F)(F)C1]

The title compound (8a) was obtained from 4-chloro-2-iodo-6-methylbenzoic acid (100 mg; 0.337 mmol) and from 3,3-difluoroazetidine (48 mg; 0.371 mmol) according to the General Procedure I in 56% yield (70 mg; 0.189 mmol). The crude product was purified by flash column chromatography on silica (hexane/AcOEt, 100:0 to 0:100, v/v, 22 min, 10 mL/min).

ESI-MS m/z for C₁₁H₁₀ClF₂INO found 371.8/372.7 [M+H]⁺; Rₜ=1.44 min (Method A); ¹H NMR (700 MHz, CDCl₃) δ 7.70-7.67 (m, 1H), 7.26-7.22 (m, 1H), 4.63-4.56 (m, 1H), 4.56-4.50 (m, 1H), 4.37-4.25 (m, 1H), 4.11-3.98 (m, 1H), 2.36 (s, 3H).

### Step 2.

### Synthesis of (4-chloro-2-methyl-6-(4,4,5,5-tetramethyl-1,3,2-dioxaborolan-2-yl)phenyl)(3,3-difluoroazetidin-1-yl)methanone (8b)

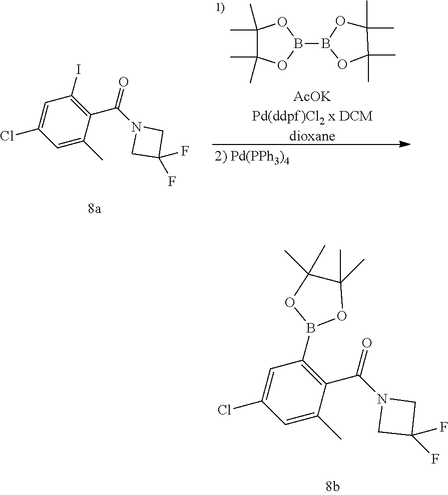

In a Schlenk flask was placed the compound 8a (70 mg; 0.189 mmol), bis(pinacolato)diboron (96 mg; 0.377 mmol), AcOK (55 mg; 0.565 mmol) and Pd(dppf)Cl₂×DCM (15 mg; 0.019 mmol) and dioxane (1.5 mL). The reaction mixture was intensively flushed with Ar. The mixture was capped and stirred at 90° C. overnight. Then Pd(PPh₃)₄ (11 mg; 0.009 mmol) was added and stirred at 90° C. overnight. Then another portion of bis(pinacolato)diboron (96 mg; 0.377 mmol) and Pd(dppf)Cl₂×DCM (15 mg; 0.019 mmol) was added and stirred at 90° C. overnight and after that another portion of Pd(PPh₃)₄ (11 mg; 0.009 mmol) was added and stirred at 90° C. for 2 days. The reaction progress was monitored by LC-MS. When analysis indicated completion of the reaction, the reaction mixture was concentrated in vacuo and the residue was purified by flash column chromatography on silica (hexane/AcOEt, 100:0 to 0:100, v/v, 28 min, 10 mL/min) and then was used to the next step without any additional purification.

ESI-MS m/z for C₁₇H₂₂BClF2NO₃ found 371.9/373.9 [M+H]⁺; Rₜ=1.73 min (Method A)

### Step 3.

### Synthesis of 3-((4-(5-chloro-2-(3,3-difluoroazetidine-1-carbonyl)-3-methylphenyl)pyrrolo[2,1-f][1,2,4]triazin-6-yl)methyl)-6,6-dimethyl-3-azabicyclo[3.1.0]hexane-2,4-dione hydrochloride (8)

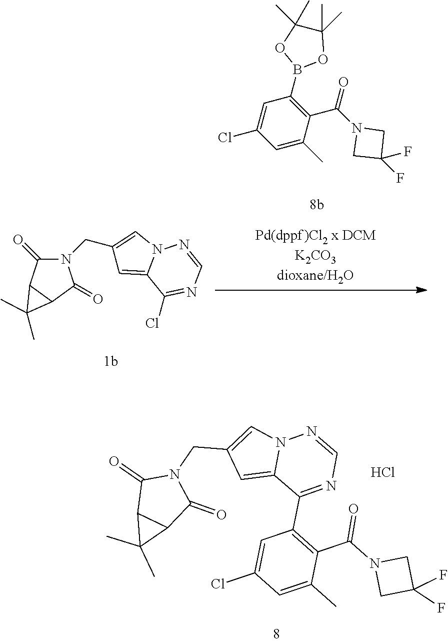 [SMILES: Cc1cc(Cl)cc(B2OC(C)(C)C(C)(C)O2)c1C(=O)N1CC(F)(F)C1 ; CC1(C)C2C(=O)N(Cc3cc4c(Cl)ncnn4c3)C(=O)C21 ; Cc1cc(Cl)cc(-c2ncnn3cc(CN4C(=O)C5C(C4=O)C5(C)C)cc23)c1C(=O)N1CC(F)(F)C1 ; Cl]

The title compound (8) was obtained as a single isomer as a hydrochloride salt from 1b (57 mg; 0.188 mmol) and from 8b (the crude product) according to the General Procedure IV in 7% yield (8 mg; 0.014 mmol). The crude product was purified by flash column chromatography on silica (hexane/AcOEt, 100:0 to 0:100, v/v, 27 min, 10 mL/min) and then by preparative reversed-phase column chromatography (C-18, water+0.3‰ HCl (36%)/MeCN, 90:10 to 10:90, 30 min, 20 mL/min, Rₜ=25.4 min).

ESI-MS m/z for C₂₅H₂₃ClF₂N₅O₃ found 514.2/516.2 [M+H]⁺; Rₜ=1.59 min (Method A); ¹H NMR (700 MHz, Methanol-d₄) δ 8.44 (s, 1H), 8.03-8.00 (m, 1H), 7.83-7.79 (m, 1H), 7.63-7.59 (m, 1H), 6.96-6.91 (m, 1H), 4.75-4.68 (m, 2H), 4.67-4.61 (m, 1H), 4.46-4.41 (m, 2H), 4.37-4.28 (m, 1H), 2.48-2.46 (m, 2H), 2.45 (s, 3H), 1.24 (s, 3H), 1.04 (s, 3H).

### Example 9

### Synthesis of 3-((4-(2-(2-aminoethoxy)-5-chloro-3-methylphenyl)pyrrolo[2,1-f][1,2,4]triazin-6-yl)methyl)-6,6-dimethyl-3-azabicyclo[3.1.0]hexane-2,4-dione hydrochloride (9)

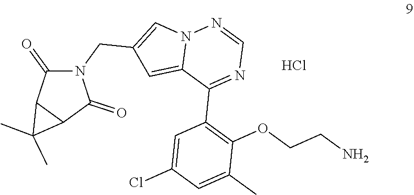 [SMILES: Cc1cc(Cl)cc(-c2ncnn3cc(CN4C(=O)C5C(C4=O)C5(C)C)cc23)c1OCCN ; Cl]

### Step 1.

### Synthesis of 1-bromo-5-chloro-2-(methoxymethoxy)-3-methylbenzene (9a)

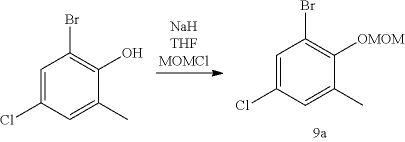

To a solution of 2-bromo-4-chloro-6-methylphenol (118 mg; 0.533 mmol) in THF (15 mL) NaH (60% in mineral oil; 32 mg; 0.800 mmol) was added at 0° C. and stirred for 1 hour. Then to this mixture MOMCl (61 μL; 0.799 mmol) was added dropwise and a whole was stirred at room temperature overnight. Then, a saturated aqueous solution of NH₄Cl was added to the reaction mixture under ice-cooling and the product was extracted with AcOEt. The combined organic layers were washed with brine and dried over anhydrous Na₂SO₄, filtered and concentrated in vacuo. The crude product was used to the next step without further purification. Compound 9a was obtained in 99% yield (140 mg; 0.530 mmol).

### Step 2.

### Synthesis of 2-(5-chloro-2-(methoxymethoxy)-3-methylphenyl)-4,4,5,5-tetramethyl-1,3,2-dioxaborolane (9b)

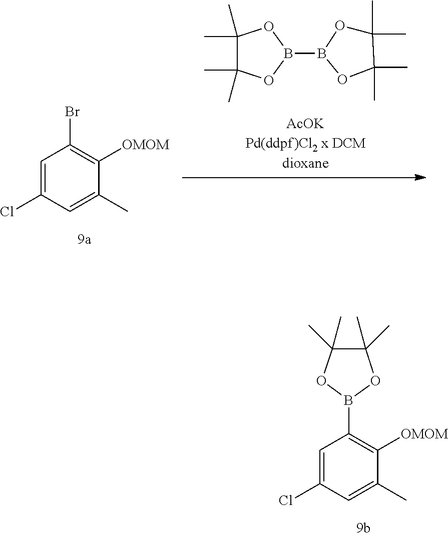

In a Schlenk flask was placed the compound 9a (140 mg; 0.530 mmol), bis(pinacolato)diboron (268 mg; 1.054 mmol), AcOK (155 mg; 1.581 mmol) and Pd(dppf)Cl₂×DCM (43 mg; 0.053 mmol) and dioxane (2 mL). The reaction mixture was intensively flushed with Ar. The mixture was capped and stirred at 90° C. overnight. The reaction progress was monitored by LC-MS. When analysis indicated completion of the reaction, the reaction mixture was concentrated in vacuo and the residue was purified by flash column chromatography on silica (hexane/AcOEt, 100:0 to 70:30, v/v, 20 min. Compound 9b was obtained in 58% yield (96 mg; 0.308 mmol).

ESI-MS m/z for C₁₆H₂₃BC106 found 371.9/373.9 [M−H+HCOOH]⁻; Rₜ=1.93 min (Method A)

### Step 3.

### Synthesis of 3-((4-(5-chloro-2-(methoxymethoxy)-3-methylphenyl)pyrrolo[2,1-f][1,2,4]triazin-6-yl)methyl)-6,6-dimethyl-3-azabicyclo[3.1.0]hexane-2,4-dione (9c)

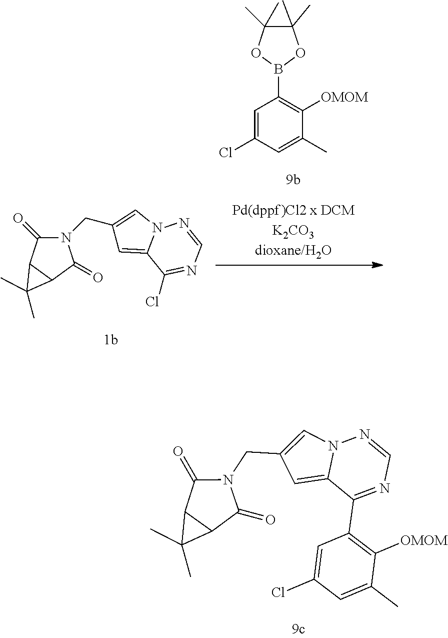

The title compound (9c) was obtained from 1b (179 mg; 0.589 mmol) and from 9b (92 mg; 0.294 mmol) according to the General Procedure IV in 98% yield (131 mg; 0.288 mmol). The crude product was purified by flash column chromatography on silica (hexane/AcOEt, 100:0 to 0:100, v/v, 20 min).

ESI-MS m/z for C₂₃H₂₄ClN₄O₄ found 455.0/457.0 [M+H]⁺; Rₜ=1.59 min (Method A)

### Step 4.

### Synthesis of 3-((4-(5-chloro-2-hydroxy-3-methylphenyl)pyrrolo[2,1-f][1,2,4]triazin-6-yl)methyl)-6,6-dimethyl-3-azabicyclo[3.1.0]hexane-2,4-dione (9d)

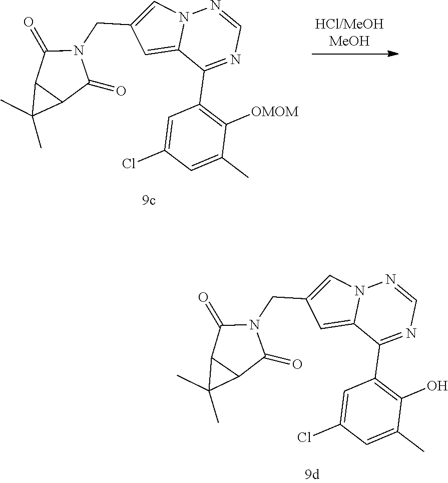

The solution of 9c (93 mg; 0.204 mmol) and 4 M HCl/MeOH (3.5 mL) in MeOH (2.5 mL) was stirred at room temperature overnight. The reaction progress was monitored by LC-MS. When analysis indicated completion of the reaction, the solvent was evaporated in vacuo and the residue was purified by flash column chromatography on silica (hexane/AcOEt, 100:0 to 0:100, v/v, 20 min). Compound 9d was obtained in 62% yield (52 mg; 0.127 mmol).

ESI-MS m/z for C₂₁H₂₀ClN₄O₃ found 411.0/413.0 [M+H]⁻; Rₜ=1.82 min (Method A)

### Step 5.

### Synthesis of tert-butyl (2-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenoxy)ethyl)carbamate (9e)

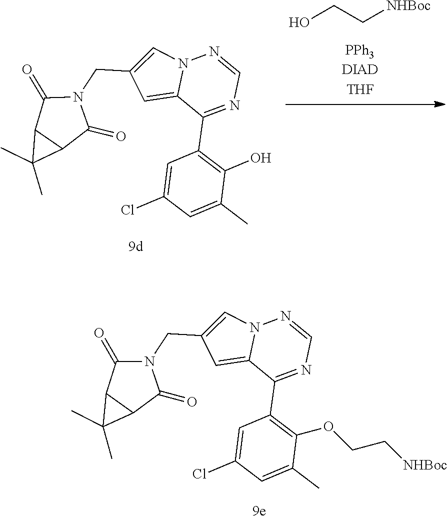 [SMILES: CC(C)(C)OC(=O)NCCO ; Cc1cc(Cl)cc(-c2ncnn3cc(CN4C(=O)C5C(C4=O)C5(C)C)cc23)c1O ; Cc1cc(Cl)cc(-c2ncnn3cc(CN4C(=O)C5C(C4=O)C5(C)C)cc23)c1OCCNC(=O)OC(C)(C)C]

To a cooled to −10° C. solution of 9d (25 mg; 0.062 mmol), tert-butyl (2-hydroxyethyl)carbamate (12 mg; 0.074 mmol) and PPh₃ (20 mg; 0.074 mmol) in THE (0.4 mL) DIAD (16 μL; 0.081 mmol) was slowly added. The resulting mixture was stirred at room temperature overnight. The reaction progress was monitored by LC-MS. When analysis indicated almost completion of the reaction, the solvent was evaporated in vacuo and the residue was purified by flash column chromatography on silica (hexane/AcOEt, 100:0 to 20:80, v/v, 30 min). Compound 9e was obtained in 82% yield (28 mg; 0.051 mmol).

ESI-MS m/z for C₂₈H₃₃ClN₅O₅ found 554.0/556.0 [M+H]⁺; Rₜ=1.76 min (Method A)

### Step 6.

### Synthesis of 3-((4-(2-(2-aminoethoxy)-5-chloro-3-methylphenyl)pyrrolo[2,1-f][1,2,4]triazin-6-yl)methyl)-6,6-dimethyl-3-azabicyclo[3.1.0]hexane-2,4-dione hydrochloride (9)

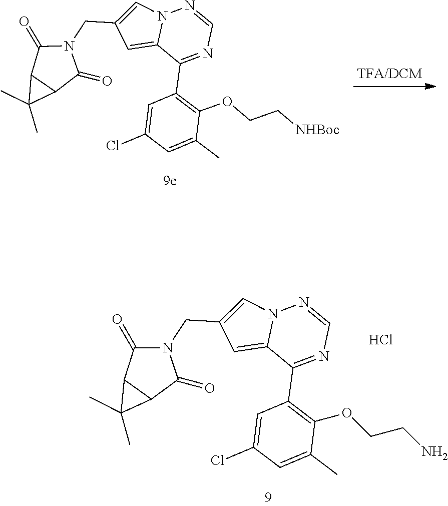 [SMILES: Cc1cc(Cl)cc(-c2ncnn3cc(CN4C(=O)C5C(C4=O)C5(C)C)cc23)c1OCCNC(=O)OC(C)(C)C ; Cc1cc(Cl)cc(-c2ncnn3cc(CN4C(=O)C5C(C4=O)C5(C)C)cc23)c1OCCN ; Cl]

The title compound (9) was obtained as a hydrochloride salt from 9e (28 mg; 0.051 mmol) according to the General Procedure IIIb in 35% yield (9 mg; 0.018 mmol). The crude product was purified by preparative reversed-phase column chromatography (C-18, water+0.3‰ HCl (36%)/MeCN, 99:1 to 40:60, 30 min, 20 mL/min, Rₜ=19.4 min).

ESI-MS m/z for C₂₃H₂₅ClN₅O₃ found 454.3/456.3 [M+H]⁺; Rₜ=1.03 min (Method A); ¹H NMR (700 MHz, D₂O) δ 8.43 (s, 1H), 8.01-7.98 (m, 1H), 7.52-7.46 (m, 1H), 7.32-7.29 (m, 1H), 6.81-6.73 (m, 1H), 4.67-4.59 (m, 2H), 3.72 (t, J=5.1 Hz, 2H), 2.96 (t, J=5.1 Hz, 2H), 2.55 (s, 2H), 2.32 (s, 3H), 1.17 (s, 3H), 0.93 (s, 3H).

### Example 10

### Synthesis of 3-((4-(5-chloro-1-((4-fluoropiperidin-4-yl)methyl)-1H-indol-7-yl)pyrrolo[2,1-f][1,2,4]triazin-6-yl)methyl)-6,6-dimethyl-3-azabicyclo[3.1.0]hexane-2,4-dione hydrochloride (10)

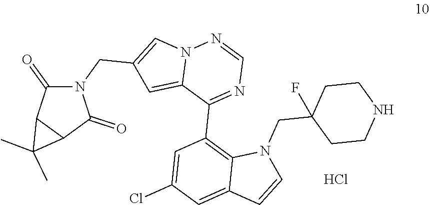 [SMILES: CC1(C)C2C(=O)N(Cc3cc4c(-c5cc(Cl)cc6ccn(CC7(F)CCNCC7)c56)ncnn4c3)C(=O)C21 ; Cl]

### Step 1.

### Synthesis of tert-butyl 4-((7-bromo-5-chloro-1H-indol-1-yl)methyl)-4-hydroxypiperidine-1-carboxylate (10a)

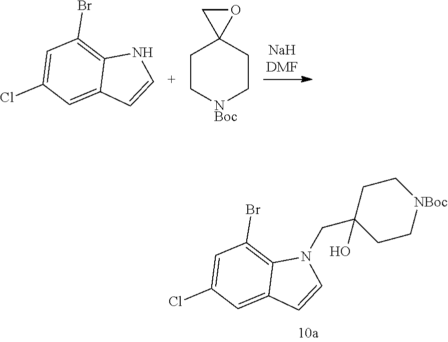

To the solution of 7-bromo-5-chloro-1H-indole (113 mg; 0.49 mmol), tert-butyl 1-oxa-6-azaspiro[2.5]octane-6-carboxylate (136 mg; 0.64 mmol) in dry DMF (2 mL) NaH (60% in mineral oil; 31 mg; 1.28 mmol) was added and heated at 70° C. for 1 hour. The reaction progress was monitored by LC-MS. When analysis indicated completion of the reaction, a whole was cooled to room temperature and concentrated in vacuo. The crude product was then purified by flash column chromatography on silica (hexane/AcOEt, 100:0 to 50:50, v/v, 22 min). Compound 10a was obtained in 78% yield (168 mg; 0.38 mmol).

ESI-MS m/z for C₁₉H₂₅BrClN₂O₃ found 443.0/445.0 [M+H]⁺; Rₜ=1.84 min (Method A)

### Step 2.

### Synthesis of tert-butyl 4-((7-bromo-5-chloro-1H-indol-1-yl)methyl)-4-fluoropiperidine-1-carboxylate (10b)

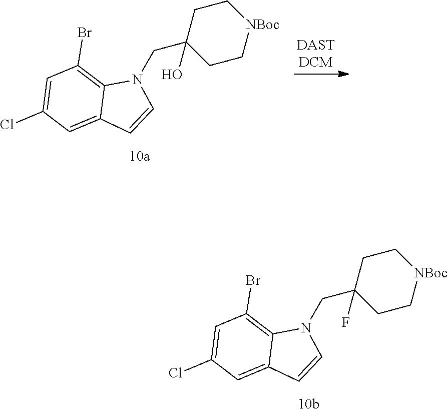 [SMILES: CC(C)(C)OC(=O)N1CCC(O)(Cn2ccc3cc(Cl)cc(Br)c32)CC1 ; CC(C)(C)OC(=O)N1CCC(F)(Cn2ccc3cc(Cl)cc(Br)c32)CC1]

To a cooled to −20° C. solution of 10a (330 mg; 0.746 mmol) in dry DCM (22 mL) DAST (0.19 mL; 1.420 mmol) was added dropwise over 5 minutes. Then, this mixture was stirred at 0° C. for 2 hours. The reaction progress was monitored by LC-MS. When analysis indicated completion of the reaction, the reaction was quenched with 5% NaHCO₃ at 0° C. and stirred for 30 minutes. The phases were separated and an aqueous one was extracted with DCM (2×20 mL). The combined organic solutions were washed with water (30 mL) and brine (30 mL) and then dried over anhydrous Na₂SO₄, filtered and concentrated in vacuo. The residue was purified by flash column chromatography on silica (hexane/AcOEt, 100:0 to 50:50, v/v, 25 min). Compound 10b was obtained in 35% yield (117 mg; 0.263 mmol).

ESI-MS m/z for C₁₅H₁₆BrClFN₂O₂ found 388.8/390.8 [M+H-tBu]⁺; Rₜ=2.11 min (Method A); ¹H NMR (700 MHz, CDCl₃) δ 7.59-7.56 (m, 1H), 7.41-7.38 (m, 1H), 7.25-7.22 (m, 1H), 6.51 (d, J=3.3 Hz, 1H), 5.00-4.65 (m, 2H), 4.09-3.87 (m, 2H), 3.02-2.90 (m, 2H), 1.79-1.64 (m, 2H), 1.47 (s, 11H).

### Step 3.

### Synthesis of tert-butyl 4-((5-chloro-7-(4,4,5,5-tetramethyl-1,3,2-dioxaborolan-2-yl)-1H-indol-1-yl)methyl)-4-fluoropiperidine-1-carboxylate (10c)

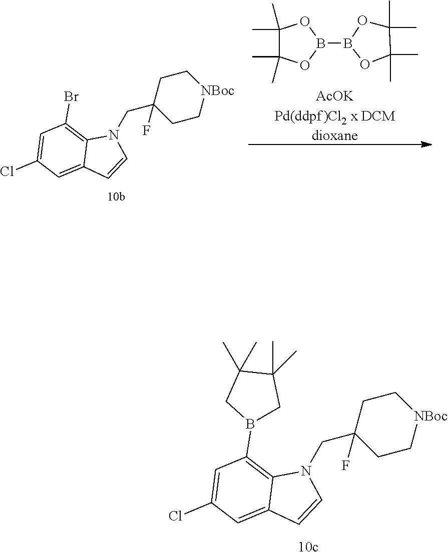

In a Schlenk flask was placed the compound 10b (115 mg; 0.257 mmol), bis(pinacolato)diboron (130 mg; 0.514 mmol), AcOK (76 mg; 0.771 mmol), Pd(dppf)Cl₂×DCM (21 mg; 0.026 mmol) and dioxane (2 mL). The reaction mixture was intensively flushed with Ar. The mixture was capped and stirred at 90° C. overnight. The reaction progress was monitored by LC-MS. When analysis indicated completion of the reaction, the reaction mixture was concentrated in vacuo and the crude product was used to the next step without any additional purification.

ESI-MS m/z for C₂₁H₂₈BClFN₂O₄ found 437.1/439.1 [M+H-tBu]⁺; Rₜ=2.26 min (Method A)

### Step 4.

### Synthesis of tert-butyl 4-((5-chloro-7-(6-((6,6-dimethyl-2,4-dioxo-3-azabicyclo[3.1.0]hexan-3-yl)methyl)pyrrolo[2,1-f][1,2,4]triazin-4-yl)-1H-indol-1-yl)methyl)-4-fluoropiperidine-1-carboxylate (10d)

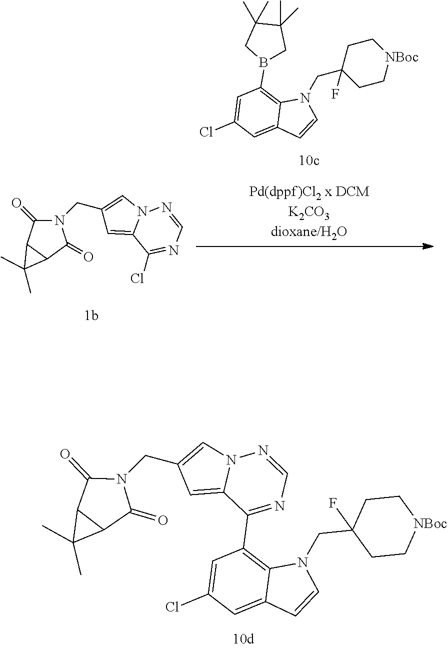 [SMILES: CC(C)(C)OC(=O)N1CCC(F)(Cn2ccc3cc(Cl)cc(B4CC(C)(C)C(C)(C)C4)c32)CC1 ; CC1(C)C2C(=O)N(Cc3cc4c(Cl)ncnn4c3)C(=O)C21 ; CC(C)(C)OC(=O)N1CCC(F)(Cn2ccc3cc(Cl)cc(-c4ncnn5cc(CN6C(=O)C7C(C6=O)C7(C)C)cc45)c32)CC1]

The title compound (10d) was obtained from 1b (78 mg; 0.257 mmol) and from 10c (the crude product) according to the General Procedure IV in 43% yield (per two steps)(70 mg; 0.110 mmol). The crude product was purified by flash column chromatography on silica (hexane/AcOEt, 100:0 to 0:100, v/v, 27 min).

ESI-MS m/z for C₃₃H₃₇ClFN₆O₄ found 635.3/637.2 [M+H]⁺; Rₜ=1.93 min (Method A); ¹H NMR (700 MHz, CDCl₃) δ 8.51 (s, 1H), 7.98-7.94 (m, 1H), 7.82-7.76 (m, 1H), 7.44-7.41 (m, 1H), 7.21-7.18 (m, 1H), 6.80-6.75 (m, 1H), 6.66-6.62 (m, 1H), 4.71-4.64 (m, 2H), 4.30-4.20 (m, 2H), 4.05-3.96 (m, 2H), 3.82-3.70 (m, 2H), 2.77-2.68 (m, 2H), 2.38-2.32 (m, 2H), 1.43 (s, 9H), 1.27-1.30 (m, 2H), 1.24 (s, 3H), 1.08 (s, 3H).

### Step 5.

### Synthesis of 3-((4-(5-chloro-1-((4-fluoropiperidin-4-yl)methyl)-1H-indol-7-yl)pyrrolo[2,1-f][1,2,4]triazin-6-yl)methyl)-6,6-dimethyl-3-azabicyclo[3.1.0]hexane-2,4-dione hydrochloride (10)

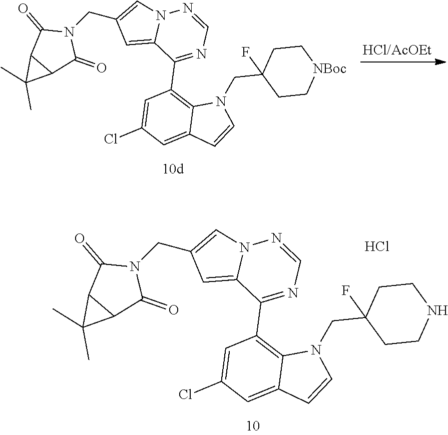 [SMILES: CC(C)(C)OC(=O)N1CCC(F)(Cn2ccc3cc(Cl)cc(-c4ncnn5cc(CN6C(=O)C7C(C6=O)C7(C)C)cc45)c32)CC1 ; CC1(C)C2C(=O)N(Cc3cc4c(-c5cc(Cl)cc6ccn(CC7(F)CCNCC7)c56)ncnn4c3)C(=O)C21 ; Cl]

The title compound (10) was obtained as a hydrochloride salt from 10d (62 mg; 0.098 mmol) according to the General Procedure IIIa in 31% yield (17 mg; 0.030 mmol). The crude product was purified by preparative reversed-phase column chromatography (C-18, water+0.3‰ HCl (36%)/MeCN, 95:5 to 20:80, 30 min, 20 mL/min, Rₜ=16.5 min).

ESI-MS m/z for C₂₈H₂₉ClFN₆O₂ found 535.4/537.4 [M+H]⁺; Rₜ=1.13 min (Method A); ¹H NMR (700 MHz, Methanol-d₄) δ 8.65 (s, 1H), 8.19-8.16 (m, 1H), 7.91-7.87 (m, 1H), 7.47-7.45 (m, 1H), 7.44-7.42 (m, 1H), 6.85-6.82 (m, 1H), 6.77-6.73 (m, 1H), 4.74-4.67 (m, 2H), 4.46-4.38 (m, 2H), 3.21-3.15 (m, 2H), 2.95-2.81 (m, 2H), 2.48 (s, 2H), 1.82-1.67 (m, 2H), 1.49-1.40 (m, 2H), 1.25 (s, 3H), 1.09 (s, 3H).

### Example 11

### Synthesis of 3-((4-(5-chloro-2-(((2S,6S)-2,6-dimethylpiperazin-1-yl)methyl)-3-methylphenyl)pyrrolo[2,1-f][1,2,4]triazin-6-yl)methyl)-6,6-dimethyl-3-azabicyclo[3.1.0]hexane-2,4-dione dihydrochloride (11)

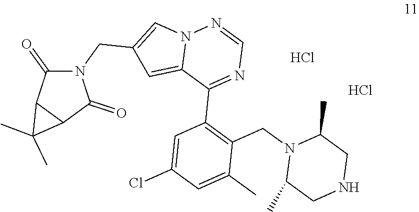 [SMILES: Cc1cc(Cl)cc(-c2ncnn3cc(CN4C(=O)C5C(C4=O)C5(C)C)cc23)c1CN1[C@@H](C)CNC[C@@H]1C ; Cl ; Cl]

### Step 1.

### Synthesis of 5-chloro-1-iodo-2-((methoxymethoxy)methyl)-3-methylbenzene (11a)

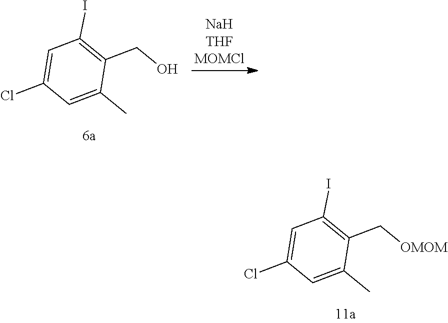

To a solution of 6a (200 mg; 0.709 mmol) in THF (15 mL) NaH (60% in mineral oil; 71 mg; 1.770 mmol) was added at 0° C. and stirred for 1 hour. Then to this mixture MOMCl (81 μL; 1.064 mmol) was added dropwise and a whole was stirred at room temperature overnight. Then the reaction mixture was quenched with MeOH. A whole was concentrated in vacuo. The residue was purified by flash column chromatography on silica (hexane/AcOEt, 100:0 to 0:100, v/v, 27 min, 20 mL/min). Compound 11a was obtained in 69% yield (160 mg; 0.491 mmol).

¹H NMR (700 MHz, CDCl₃) δ 7.77-7.72 (m, 1H), 7.21-7.16 (m, 1H), 4.81-4.71 (m, 4H), 3.48 (s, 3H), 2.48 (s, 3H).

### Step 2.

### Synthesis of 2-(5-chloro-2-((methoxymethoxy)methyl)-3-methylphenyl)-4,4,5,5-tetramethyl-1,3,2-dioxaborolane (11b)

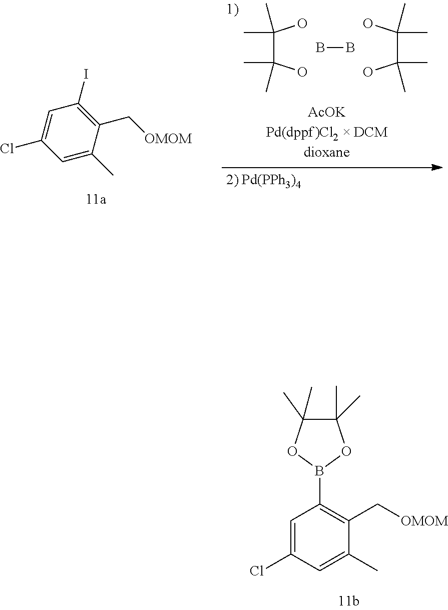

In a Schlenk flask was placed the compound 11a (80 mg; 0.245 mmol), bis(pinacolato)diboron (124 mg; 0.490 mmol), AcOK (72 mg; 0.735 mmol) and Pd(dppf)Cl₂×DCM (20 mg; 0.024 mmol) and dioxane (2 mL). The reaction mixture was intensively flushed with Ar. The mixture was capped and stirred at 90° C. overnight. Then Pd(PPh₃)₄ (28 mg; 0.025 mmol) was added and stirred at 90° C. overnight. Then another portion of Pd(PPh₃)₄ (28 mg; 0.025 mmol) was added and stirred at 90° C. for 2 days. The reaction progress was monitored by LC-MS. When analysis indicated completion of the reaction, the reaction mixture was concentrated in vacuo and the residue was purified by flash column chromatography on silica (hexane/AcOEt, 100:0 to 0:100, v/v, 27 min, 10 mL/min) and then was used to the next step without any additional purification.

### Step 3.

### Synthesis of 3-((4-(5-chloro-2-((methoxymethoxy)methyl)-3-methylphenyl)pyrrolo[2,1-f][1,2,4]triazin-6-yl)methyl)-6,6-dimethyl-3-azabicyclo[3.1.0]hexane-2,4-dione (11c)

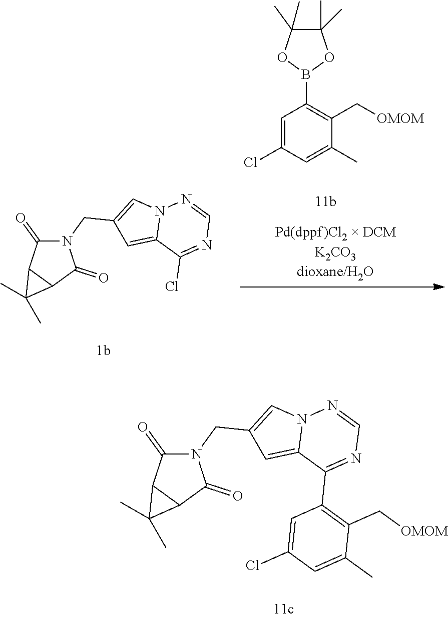

The title compound (11c) was obtained from 1b (74 mg; 0.245 mmol) and from 11b (the crude product) according to the General Procedure IV in 58% yield (per two steps)(67 mg; 0.143 mmol). The crude product was purified by flash column chromatography on silica (hexane/AcOEt, 100:0 to 0:100, v/v, 27 min, 10 mL/min).

ESI-MS m/z for C₂₄H₂₆ClN₄O₄ found 469.0/470.9 [M+H]⁺; Rₜ=1.56 min (Method A); ¹H NMR (700 MHz, CDCl₃) δ 8.50 (s, 1H), 7.95-7.93 (m, 1H), 7.40-7.38 (m, 1H), 7.33-7.30 (m, 1H), 6.66-6.63 (m, 1H), 4.65 (s, 2H), 4.56 (s, 2H), 4.45 (s, 2H), 3.17 (s, 3H), 2.52 (s, 3H), 1.93 (s, 2H), 1.24 (s, 3H), 1.07 (s, 3H).

### Step 4.

### Synthesis of 3-((4-(5-chloro-2-(hydroxymethyl)-3-methylphenyl)pyrrolo[2,1-f][1,2,4]triazin-6-yl)methyl)-6,6-dimethyl-3-azabicyclo[3.1.0]hexane-2,4-dione (11d)

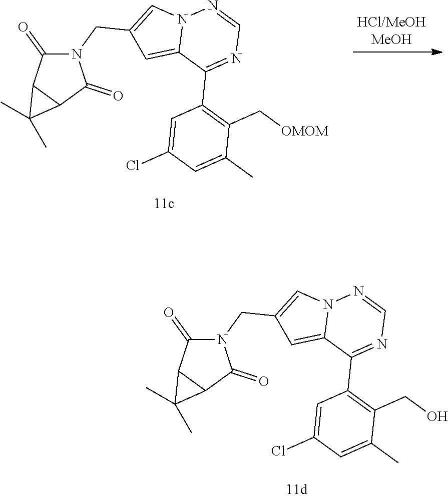

To the solution of 11c (60 mg; 0.128 mmol) in MeOH (2 mL) 4 M HCl/MeOH was added at 0° C. and then it was stirred at room temperature overnight. The reaction progress was monitored by LC-MS. When analysis indicated completion of the reaction, When analysis indicated completion of the reaction, the reaction mixture was concentrated in vacuo and the crude product was used to the next step without any additional purification.

ESI-MS m/z for C₂₁H₂₂ClN₄O₃ found 425.0/426.9 [M+H]⁺; Rₜ=1.41 min (Method A); ¹H NMR (700 MHz, Methanol-d₄) δ 8.70 (s, 1H), 8.53 (s, 1H), 7.71-7.61 (m, 1H), 7.55-7.48 (m, 1H), 7.26 (s, 1H), 4.74 (s, 2H), 4.67 (s, 2H), 2.52 (s, 3H), 2.49 (s, 2H), 1.26 (s, 3H), 1.10 (s, 3H).

### Step 5.

### Synthesis of 4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylbenzyl methanesulfonate (11e)

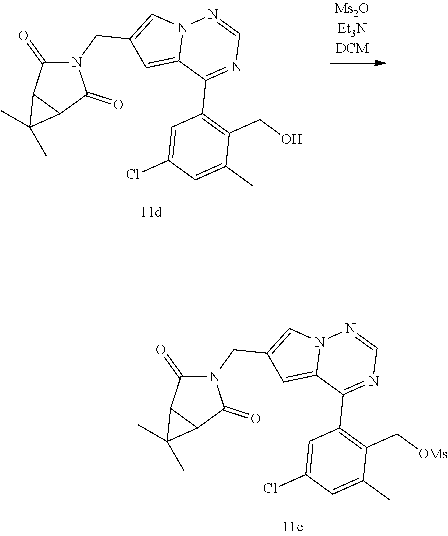 [SMILES: Cc1cc(Cl)cc(-c2ncnn3cc(CN4C(=O)C5C(C4=O)C5(C)C)cc23)c1CO ; Cc1cc(Cl)cc(-c2ncnn3cc(CN4C(=O)C5C(C4=O)C5(C)C)cc23)c1COS(C)(=O)=O]

The title compound (11e) was obtained from 11d (the crude product) according to the General Procedure V and after standard work-up the crude product was taken to the next step.

ESI-MS m/z for C₂₃H₂₄ClN₄O₅S found 503.2/504.9 [M+H]⁺; Rₜ=1.49 min (Method A)

### Step 6.

### Synthesis of tert-butyl (3S,5S)-4-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylbenzyl)-3,5-dimethylpiperazine-1-carboxylate (11f)

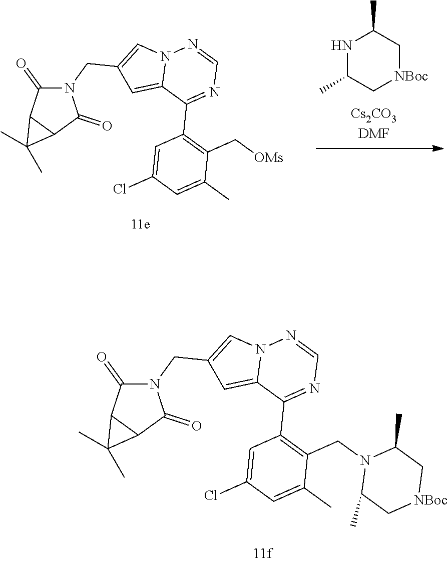 [SMILES: Cc1cc(Cl)cc(-c2ncnn3cc(CN4C(=O)C5C(C4=O)C5(C)C)cc23)c1COS(C)(=O)=O ; C[C@H]1CN(C(=O)OC(C)(C)C)C[C@H](C)N1 ; Cc1cc(Cl)cc(-c2ncnn3cc(CN4C(=O)C5C(C4=O)C5(C)C)cc23)c1CN1[C@@H](C)CN(C(=O)OC(C)(C)C)C[C@@H]1C]

The solution of 11e (the crude product), tert-butyl (3S,5S)-3,5-dimethylpiperazine-1-carboxylate (11 mg; 0.053 mmol) and Cs₂CO₃ (35 mg; 0.106 mmol) in DMF (1 mL) was stirred under argon atmosphere at room temperature overnight and then at 50° C. for 4 hours. The reaction progress was monitored by LC-MS. When analysis indicated completion of the reaction, the reaction mixture was diluted with AcOEt (15 mL) and washed with water (15 mL). The layers were separated and an organic one was dried over anhydrous Na₂SO₄, filtered and concentrated in vacuo. The crude product was used to the next step without any additional purification.

ESI-MS m/z for C₃₃H₄₂ClN₆O₄ found 621.1/623.1 [M+H]⁺; Rₜ=1.52 min (Method A)

### Step 7.

### Synthesis of 3-((4-(5-chloro-2-(((2S,6S)-2,6-dimethylpiperazin-1-yl)methyl)-3-methylphenyl)pyrrolo[2,1-f][1,2,4]triazin-6-yl)methyl)-6,6-dimethyl-3-azabicyclo[3.1.0]hexane-2,4-dione dihydrochloride (11)

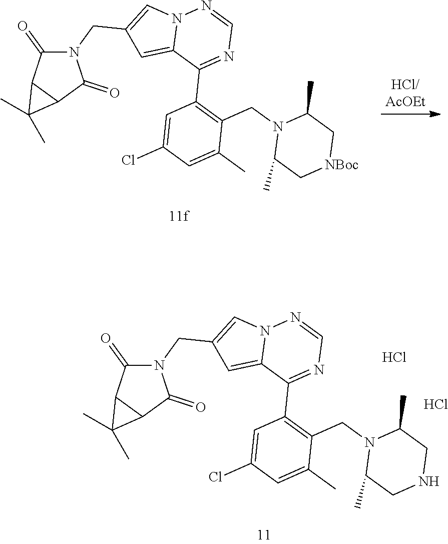 [SMILES: Cc1cc(Cl)cc(-c2ncnn3cc(CN4C(=O)C5C(C4=O)C5(C)C)cc23)c1CN1[C@@H](C)CN(C(=O)OC(C)(C)C)C[C@@H]1C ; Cc1cc(Cl)cc(-c2ncnn3cc(CN4C(=O)C5C(C4=O)C5(C)C)cc23)c1CN1[C@@H](C)CNC[C@@H]1C ; Cl ; Cl]

The title compound (11) was obtained as a dihydrochloride salt from 11f (the crude product) according to the General Procedure IIIa in 4% yield (per four steps)(3 mg; 0.005 mmol). The crude product was purified by preparative reversed-phase column chromatography (column: Cosmosil Cholester 20×250 mm, water+0.3‰ HCl (36%)/MeCN, 95:5 to 20:80, 30 min, 20 mL/min, Rₜ=15.7 min).

ESI-MS m/z for C₂₈H₃₄ClN₆O₂ found 521.4/523.4 [M+H]⁺; Rₜ=1.14 min (Method A); ¹H NMR (700 MHz, D₂O) δ 8.54 (s, 1H), 8.13-8.11 (m, 1H), 7.61-7.56 (m, 1H), 7.53-7.46 (m, 1H), 6.90-6.86 (m, 1H), 4.67-4.62 (m, 3H), 4.35 (d, J=14.8 Hz, 1H), 4.12 (d, J=14.8 Hz, 1H), 3.46-3.35 (m, 2H), 3.13-3.03 (m, 2H), 2.99-2.94 (m, 2H), 2.55 (s, 2H), 2.49 (s, 3H), 1.17 (s, 3H), 1.07 (s, 3H), 1.06 (s, 3H), 0.99 (s, 3H).

### Example 12

### Synthesis of 3-((4-(5-chloro-3-methyl-2-(((S)-piperidin-3-yl)oxy)phenyl)-5-methylpyrrolo[2,1-f][1,2,4]triazin-6-yl)methyl)-6,6-dimethyl-3-azabicyclo[3.1.0]hexane-2,4-dione hydrochloride (12)

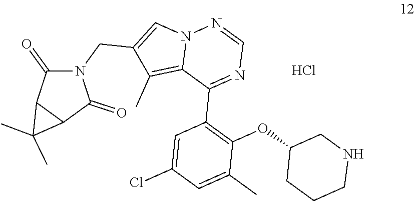 [SMILES: Cc1cc(Cl)cc(-c2ncnn3cc(CN4C(=O)C5C(C4=O)C5(C)C)c(C)c23)c1O[C@H]1CCCNC1 ; Cl]

The title compound (12) was obtained as a hydrochloride salt as a mixture of rotamers in 4% overall yield in a similar way to Example 1 with the exception that, in the first step of the synthesis, ethyl 4-chloro-5-methylpyrrolo[2,1-f][1,2,4]triazine-6-carboxylate was used instead of ethyl 4-chloropyrrolo[2,1-f][1,2,4]triazine-6-carboxylate. The crude product was purified by preparative reversed-phase column chromatography (C-18, water+0.3‰ HCl (36%)/MeCN, 95:5 to 50:50, 30 min, 20 mL/min).

ESI-MS m/z for C₂₇H₃₄ClN₅O₃ found 508.4/510.4 [M+H]⁺; Rₜ=1.10 min (Method A); ¹H NMR (700 MHz, Methanol-d₄) δ 8.55-8.52 (m, 1H), 8.32-8.28 (m, 1H), 7.66-7.62 (m, 1H), 7.44-7.39 (m, 1H), 4.75-4.61 (m, 2H), 4.20-4.06 (m, 1H), 3.13-3.01 (m, 2H), 2.98-2.82 (m, 1H), 2.53-2.47 (m, 2H), 2.47-2.42 (m, 3H), 2.10-2.01 (m, 3H), 1.84-1.70 (m, 1H), 1.68-1.46 (m, 3H), 1.40-1.28 (m, 1H), 1.28-1.24 (m, 3H), 1.10-1.04 (m, 3H).

### Example 13

### Synthesis of 3-((4-(5-chloro-3-methyl-2-(((R)-morpholin-2-yl)methyl)phenyl)-5-methylpyrrolo[2,1-f][1,2,4]triazin-6-yl)methyl)-6,6-dimethyl-3-azabicyclo[3.1.0]hexane-2,4-dione hydrochloride (13)

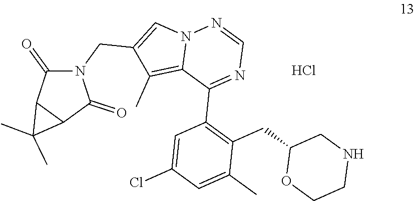 [SMILES: Cc1cc(Cl)cc(-c2ncnn3cc(CN4C(=O)C5C(C4=O)C5(C)C)c(C)c23)c1C[C@@H]1CNCCO1 ; Cl]

### Step 1.

### Synthesis of (4-chloro-5-methylpyrrolo[2,1-f][1,2,4]triazin-6-yl)methanol (13a)

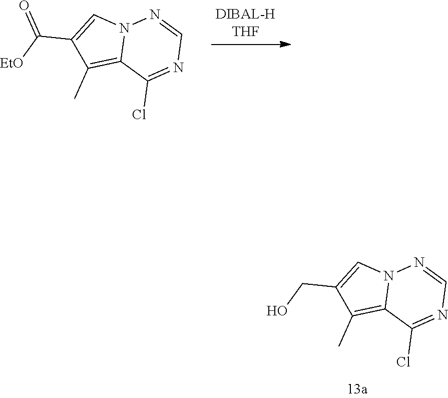 [SMILES: CCOC(=O)c1cn2ncnc(Cl)c2c1C ; Cc1c(CO)cn2ncnc(Cl)c12]

To a stirred solution of ethyl 4-chloro-5-methylpyrrolo[2,1-f][1,2,4]triazine-6-carboxylate (1.00 g; 4.17 mmol) in dry THE (100 mL) DIBAL-H (1 M in DCM; 21 mL; 20.86 mmol) was added dropwise at −78° C. Then the reaction mixture was allowed to warm to −20° C. and stirred at this temperature for 30 minutes and then at 0° C. for 1 hour. The reaction progress was monitored by LC-MS. Next the reaction mixture was quenched with a saturated solution of NH₄Cl (40 mL) and extracted with AcOEt (2×). The combined organic solutions were dried over anhydrous MgSO₄, filtered and concentrated in vacuo and the crude product was used to the next step without any additional purification. Compound 13a was obtained in 99% yield (814 mg; 4.13 mmol).

¹H NMR (700 MHz, CDCl₃) δ 8.09 (s, 1H), 7.87-7.85 (m, 1H), 4.84 (d, J=5.3 Hz, 2H), 2.63 (s, 3H).

### Step 2.

### Synthesis of 3-((4-chloro-5-methylpyrrolo[2,1-f][1,2,4]triazin-6-yl)methyl)-6,6-dimethyl-3-azabicyclo[3.1.0]hexane-2,4-dione (13b)

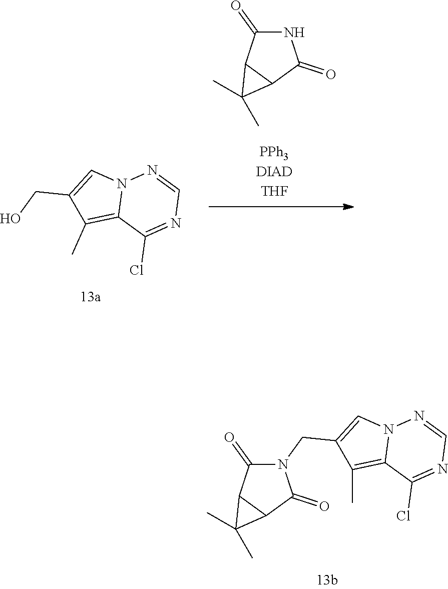

To a cooled to −10° C. solution of 13a (814 mg; 4.13 mmol), 6,6-dimethyl-3-azabicyclo[3.1.0]hexane-2,4-dione (612 mg; 4.40 mmol) and PPh₃ (1.38 g; 5.28 mmol) in THE (50 mL) DIAD (1.12 mL; 5.72 mmol) was added. The resulting mixture was stirred at room temperature for 1.5 hours. Then the reaction mixture was concentrated in vacuo and the residue was purified twice by flash column chromatography on silica (first: hexane/AcOEt, 100:0 to 20:80, v/v, 12 min, second: DCM/MeOH, 100:0 to 90:10, v/v, 12 min). Compound 13b was obtained in 24% yield (315 mg; 0.99 mmol). ¹H NMR (700 MHz, CDCl₃) δ 8.08 (s, 1H), 7.91 (s, 1H), 4.64 (s, 2H), 2.67 (s, 3H), 2.35 (s, 2H), 1.25 (s, 3H), 1.06 (s, 3H).

### Step 3.

### Synthesis of tert-butyl (2R)-2-(4-chloro-2-(6-((6,6-dimethyl-2,4-dioxo-3-azabicyclo[3.1.0]hexan-3-yl)methyl)-5-methylpyrrolo[2,1-f][1,2,4]triazin-4-yl)-6-methylbenzyl)morpholine-4-carboxylate (13c)

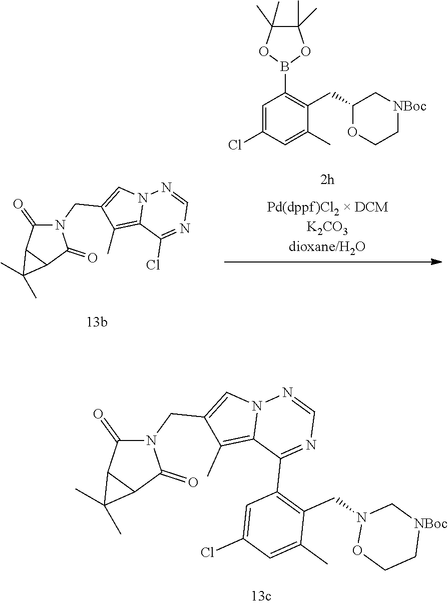

The title compound (13c) was obtained from 13b (85 mg; 0.267 mmol) and from 2h (120 mg; 0.267 mmol) according to the General Procedure IV in 10% yield (16 mg; 0.026 mmol). The crude product was purified by flash column chromatography on silica (hexane/AcOEt, 100:0 to 0:100, v/v, 12 min).

ESI-MS m/z for C₃₂H₃₉ClN₅O₅ found 608.1/610.2 [M+H]⁺; Rₜ=1.91 min (Method A)

### Step 4.

### Synthesis of 3-((4-(5-chloro-3-methyl-2-(((R)-morpholin-2-yl)methyl)phenyl)-5-methylpyrrolo[2,1-f][1,2,4]triazin-6-yl)methyl)-6,6-dimethyl-3-azabicyclo[3.1.0]hexane-2,4-dione hydrochloride (13)

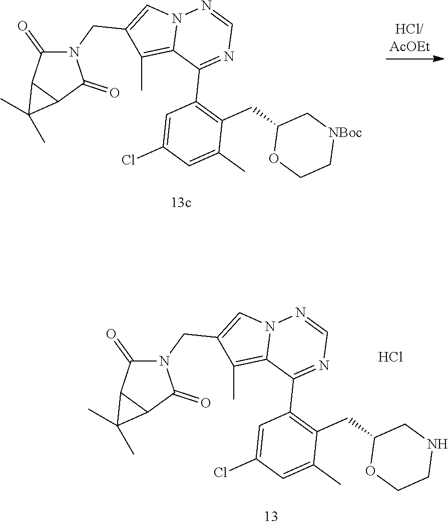 [SMILES: Cc1cc(Cl)cc(-c2ncnn3cc(CN4C(=O)C5C(C4=O)C5(C)C)c(C)c23)c1C[C@@H]1CN(C(=O)OC(C)(C)C)CCO1 ; Cc1cc(Cl)cc(-c2ncnn3cc(CN4C(=O)C5C(C4=O)C5(C)C)c(C)c23)c1C[C@@H]1CNCCO1 ; Cl]

The title compound (13) was obtained as a hydrochloride salt as a mixture of rotamers from 13c (16 mg; 0.026 mmol) according to the General Procedure IIIa in 57% yield (8 mg; 0.015 mmol). The crude product was purified by preparative reversed-phase column chromatography (C-18, water+0.3‰ HCl (36%)/MeCN, 95:5 to 50:50, 30 min, 20 mL/min).

ESI-MS m/z for C₂₇H₃₁ClN₅O₃ found 508.4/510.4 [M+H]⁺; Rₜ=1.07 min (Method A); ¹H NMR (700 MHz, Methanol-d₄) δ 8.64-8.52 (m, 1H), 8.52-8.34 (m, 1H), 7.69-7.59 (m, 1H), 7.49-7.33 (m, 1H), 4.75-4.61 (m, 2H), 3.97-3.80 (m, 1H), 3.77-3.61 (m, 1H), 3.61-3.52 (m, 1H), 3.31-2.56 (m, 7H), 2.55-2.48 (m, 4H), 2.06-1.91 (m, 3H), 1.33-1.21 (m, 3H), 1.15-0.98 (m, 3H).

### Example 14

### Synthesis of 3-((4-(5-chloro-2-(((3R,4S)-4-fluoropyrrolidin-3-yl)oxy)-3-methylphenyl)pyrrolo[2,1-f][1,2,4]triazin-6-yl)methyl)-6,6-dimethyl-3-azabicyclo[3.1.0]hexane-2,4-dione hydrochloride (14)

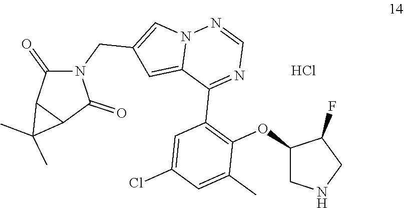 [SMILES: Cc1cc(Cl)cc(-c2ncnn3cc(CN4C(=O)C5C(C4=O)C5(C)C)cc23)c1O[C@@H]1CNC[C@@H]1F ; Cl]

The title compound (14) was obtained as a hydrochloride salt as a single isomer in 3% overall yield in a similar way to Example 1 with the exception that, in the third step of the synthesis, tert-butyl (3S,4R)-3-fluoro-4-hydroxypyrrolidine-1-carboxylate was used instead of tert-butyl (R)-3-hydroxypiperidine-1-carboxylate and in the sixth step of the synthesis, the General Procedure IIIb was used instead of the General Procedure IIIa. The crude product was purified by preparative reversed-phase column chromatography (C-18, water+0.3‰ HCl (36%)/MeCN, 98:2 to 50:50, 30 min, 20 mL/min, Rₜ=22.3 min).

ESI-MS m/z for C₂₅H₂₆ClFN₅O₃ found 498.4/500.4 [M+H]⁺; Rₜ=1.05 min (Method A); ¹H NMR (700 MHz, D₂O) δ 8.44 (s, 1H), 8.05-8.00 (m, 1H), 7.53-7.49 (m, 1H), 7.34-7.29 (m, 1H), 6.72-6.70 (m, 1H), 4.67-4.59 (m, 2H), 4.42-4.35 (m, 1H), 3.48-3.42 (m, 1H), 3.31-3.20 (m, 3H), 3.09-3.02 (m, 1H), 2.55 (s, 2H), 2.34 (s, 3H), 1.18 (s, 3H), 0.97 (s, 3H); ¹⁹F NMR (659 MHz, D₂O) δ −178.9.

### Example 15

### Synthesis of ethyl (S)-4-(5-chloro-3-methyl-2-(piperidin-3-yloxy)phenyl)pyrrolo[2,1-f][1,2,4]triazine-6-carboxylate hydrochloride (15)

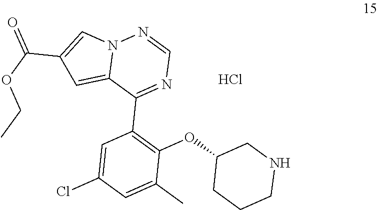 [SMILES: CCOC(=O)c1cc2c(-c3cc(Cl)cc(C)c3O[C@H]3CCCNC3)ncnn2c1 ; Cl]

### Step 1.

### Synthesis of ethyl (S)-4-(2-((1-(tert-butoxycarbonyl)piperidin-3-yl)oxy)-5-chloro-3-methylphenyl)pyrrolo[2,1-f][1,2,4]triazine-6-carboxylate (15a)

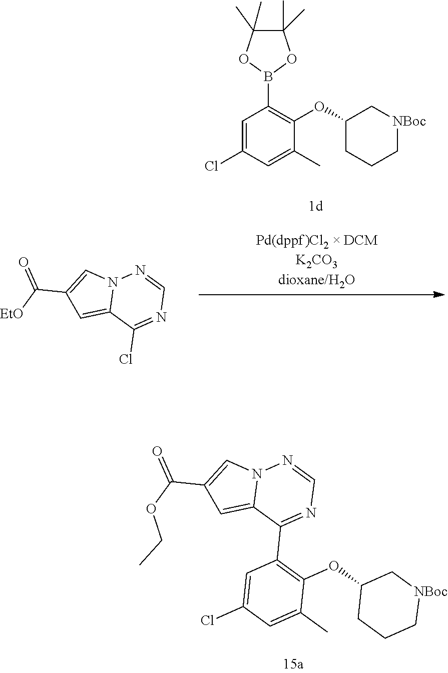 [SMILES: Cc1cc(Cl)cc(B2OC(C)(C)C(C)(C)O2)c1O[C@H]1CCCN(C(=O)OC(C)(C)C)C1 ; CCOC(=O)c1cc2c(Cl)ncnn2c1 ; CCOC(=O)c1cc2c(-c3cc(Cl)cc(C)c3O[C@H]3CCCN(C(=O)OC(C)(C)C)C3)ncnn2c1]

The title compound (15a) was obtained from ethyl 4-chloropyrrolo[2,1-f][1,2,4]triazine-6-carboxylate (100 mg; 0.443 mmol) and from 1d (200 mg; 0.443 mmol) according to the General Procedure IV in 14% yield (33 mg; 0.064 mmol). The crude product was purified by flash column chromatography on silica (hexane/AcOEt, 100:0 to 10:100, v/v, 30 min, 20 mL/min).

ESI-MS m/z for C₂₆H₃₂ClN₄O₅ found 515.3/517.1 [M+H]⁺; Rₜ=2.0 min (Method A)

### Step 2.

### Synthesis of ethyl (S)-4-(5-chloro-3-methyl-2-(piperidin-3-yloxy)phenyl)pyrrolo[2,1-f][1,2,4]triazine-6-carboxylate hydrochloride (15)

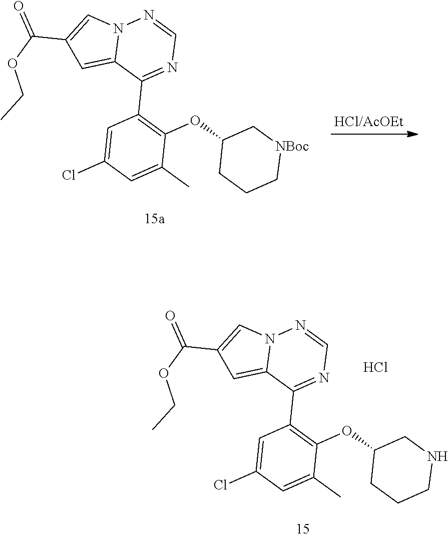 [SMILES: CCOC(=O)c1cc2c(-c3cc(Cl)cc(C)c3O[C@H]3CCCN(C(=O)OC(C)(C)C)C3)ncnn2c1 ; CCOC(=O)c1cc2c(-c3cc(Cl)cc(C)c3O[C@H]3CCCNC3)ncnn2c1 ; Cl]

The title compound (15) was obtained as a hydrochloride salt from 15a (10 mg; 0.019 mmol) according to the General Procedure IIIa in 68% yield (6 mg; 0.013 mmol). The crude product was purified by preparative reversed-phase column chromatography (C-18, water+0.3‰ HCl (36%)/MeCN, 95:5 to 20:80, 30 min, 20 mL/min, Rₜ=15.5 min).

ESI-MS m/z for C₂₁H₂₄ClN₄O₃ found 415.3/417.3 [M+H]⁺; Rₜ=1.07 min (Method A); ¹H NMR (700 MHz, Methanol-d₄) δ 8.26-8.23 (m, 1H), 7.58-7.55 (m, 1H), 7.55-7.52 (m, 1H), 7.29-7.27 (m, 1H), 6.95-6.92 (m, 1H), 4.54 (q, J=7.1 Hz, 2H), 4.28-4.22 (m, 1H), 3.21-3.02 (m, 5H), 2.47 (s, 3H), 1.86-1.72 (m, 3H), 1.59-1.54 (m, 1H), 1.49 (t, J=7.1 Hz, 3H).

### Example 16

### Synthesis of (S)-4-(5-chloro-3-methyl-2-(piperidin-3-yloxy)phenyl)pyrrolo[2,1-f][1,2,4]triazine-6-carboxylic acid hydrochloride (16)

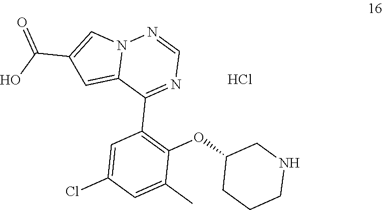 [SMILES: Cc1cc(Cl)cc(-c2ncnn3cc(C(=O)O)cc23)c1O[C@H]1CCCNC1 ; Cl]

### Step 1.

### Synthesis of (S)-4-(2-((1-(tert-butoxycarbonyl)piperidin-3-yl)oxy)-5-chloro-3-methylphenyl)pyrrolo[2,1-f][1,2,4]triazine-6-carboxylic acid (16a)

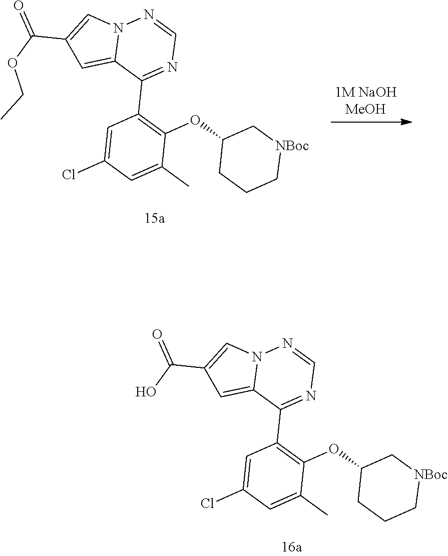 [SMILES: CCOC(=O)c1cc2c(-c3cc(Cl)cc(C)c3O[C@H]3CCCN(C(=O)OC(C)(C)C)C3)ncnn2c1 ; Cc1cc(Cl)cc(-c2ncnn3cc(C(=O)O)cc23)c1O[C@H]1CCCN(C(=O)OC(C)(C)C)C1]

To the solution of 15a (20 mg; 0.039 mmol) in MeOH (1 mL) 1 M NaOH (0.5 mL) was added. The resulting mixture was stirred at room temperature for 1 hour. Then the reaction mixture was concentrated in vacuo and the residue was dissolved in AcOEt and washed with 1 M KHSO₄ (10 mL), dried over anhydrous Na₂SO₄, filtered and concentrated in vacuo and the crude product was used to the next step without any additional purification. Compound 16a was obtained in 95% yield (18 mg; 0.037 mmol).

ESI-MS m/z for C₂₄H₂₈ClN₄O₅ found 487.1/489.1 [M+H]⁺; Rₜ=1.82 min (Method A)

### Step 2.

### Synthesis of (S)-4-(5-chloro-3-methyl-2-(piperidin-3-yloxy)phenyl)pyrrolo[2,1-f][1,2,4]triazine-6-carboxylic acid hydrochloride (16)

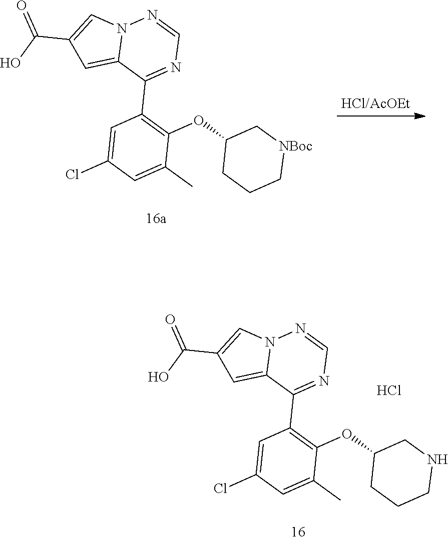 [SMILES: Cc1cc(Cl)cc(-c2ncnn3cc(C(=O)O)cc23)c1O[C@H]1CCCN(C(=O)OC(C)(C)C)C1 ; Cc1cc(Cl)cc(-c2ncnn3cc(C(=O)O)cc23)c1O[C@H]1CCCNC1 ; Cl]

The title compound (16) was obtained as a hydrochloride salt from 16a (9 mg; 0.018 mmol) according to the General Procedure IIIa in 78% yield (6 mg; 0.014 mmol). The crude product was purified by preparative reversed-phase column chromatography (C-18, water+0.3‰ HCl (36%)/MeCN, 98:2 to 30:70, 30 min, 20 mL/min, Rₜ=16.7 min).

ESI-MS m/z for C₁₉H₂₀ClN₄O₃ found 387.3/389.3 [M+H]⁺; Rₜ=0.94 min (Method A); ¹H NMR (700 MHz, Methanol-d₄) δ 8.28-8.23 (m, 1H), 7.59-7.52 (m, 2H), 7.31-7.26 (m, 1H), 6.98-6.92 (m, 1H), 4.34-4.29 (m, 1H), 3.26-3.19 (m, 1H), 3.18-3.09 (m, 2H), 3.09-3.01 (m, 1H), 2.46 (s, 3H), 1.85-1.73 (m, 3H), 1.61-1.55 (m, 1H).

### Example 17

### Synthesis of (S)-(4-(5-chloro-3-methyl-2-(piperidin-3-yloxy)phenyl)pyrrolo[2,1-f][1,2,4]triazin-6-yl)methanol hydrochloride (17)

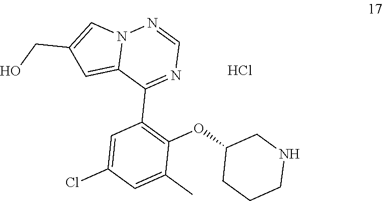 [SMILES: Cc1cc(Cl)cc(-c2ncnn3cc(CO)cc23)c1O[C@H]1CCCNC1 ; Cl]

The title compound (17) was obtained as a hydrochloride salt as a single isomer in 4% overall yield in a similar way to Example 15 with the exception that, in the first step of the synthesis, the compound 1a was used instead of ethyl 4-chloropyrrolo[2,1-f][1,2,4]triazine-6-carboxylate. The crude product was purified by preparative reversed-phase column chromatography (C-18, water+0.3‰ HCl (36%)/MeCN, 98:2 to 50:50, 30 min, 20 mL/min, Rₜ=13.5 min).

ESI-MS m/z for C₁₉H₂₂ClN₄O₂ found 373.3/375.3 [M+H]⁺; Rₜ=0.82 min (Method A); ¹H NMR (700 MHz, Methanol-d₄) δ 8.74 (s, 1H), 8.59-8.54 (m, 1H), 7.73-7.69 (m, 1H), 7.68-7.64 (m, 1H), 7.45-7.40 (m, 1H), 4.89-4.87 (m, 2H), 4.19-4.06 (m, 1H), 3.31-3.24 (m, 1H), 3.17-3.09 (m, 1H), 3.07-2.87 (m, 2H), 2.50 (s, 3H), 1.82-1.68 (m, 2H), 1.57-1.50 (m, 1H), 1.50-1.42 (m, 1H).

### Example 18

### Synthesis of (S)-4-(5-chloro-3-methyl-2-(piperidin-3-yloxy)phenyl)pyrrolo[2,1-f][1,2,4]triazine-6-carboxamide hydrochloride (18)

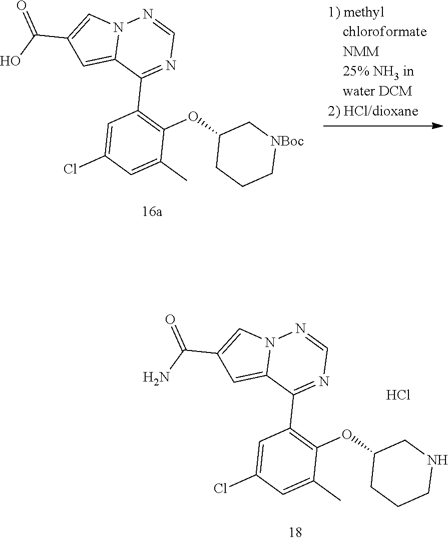 [SMILES: Cc1cc(Cl)cc(-c2ncnn3cc(C(=O)O)cc23)c1O[C@H]1CCCN(C(=O)OC(C)(C)C)C1 ; Cc1cc(Cl)cc(-c2ncnn3cc(C(N)=O)cc23)c1O[C@H]1CCCNC1 ; Cl]

The carboxylic acid 16a (27 mg; 0.055 mmol) was dissolved in dry DCM (1 mL) and to this solution N-methylmorpholine (NMM; 12 μL; 0.111 mmol) was added. The solution was cooled to −20° C. and methyl chloroformate (8.5 μL; 0.111 mmol) was added and the mixture was stirred at this temperature for additional 30 minutes. Then to this mixture 25% NH₃ in water (111 μL; 0.055 mmol) was added and a whole was allowed to warm to room temperature and stirred for 1.5 hours. Then the reaction mixture was concentrated in vacuo and the residue was treated with 4 M HCl in dioxane (3 mL) and stirred at room temperature for 1 hour. Then this mixture was concentrated in vacuo and the crude product was purified by preparative reversed-phase column chromatography (C-18, water+0.3‰ HCl (36%)/MeCN, 99:1 to 40:60, 30 min, 20 mL/min, Rₜ=15.5 min). Compound 18 was obtained as a hydrochloride salt as a single isomer in 60% yield (14 mg; 0.033 mmol).

ESI-MS m/z for C₁₉H₂₁ClN₅O₂ found 386.3/388.3 [M+H]⁺; Rₜ=0.81 min (Method A); ¹H NMR (700 MHz, D₂O) δ 8.54 (s, 1H), 8.45-8.36 (m, 1H), 7.45-7.41 (m, 1H), 7.22-7.16 (m, 1H), 7.11-7.00 (m, 1H), 3.91-3.78 (m, 1H), 3.05-2.98 (m, 1H), 2.98-2.87 (m, 3H), 2.29 (s, 3H), 1.58-1.42 (m, 2H), 1.37-1.26 (m, 2H).

### Example 19

### Synthesis of 3-((4-(1-((4-hydroxypiperidin-4-yl)methyl)-5-(trifluoromethyl)-1H-indol-7-yl)pyrrolo[2,1-f][1,2,4]triazin-6-yl)methyl)-6,6-dimethyl-3-azabicyclo[3.1.0]hexane-2,4-dione hydrochloride (19)

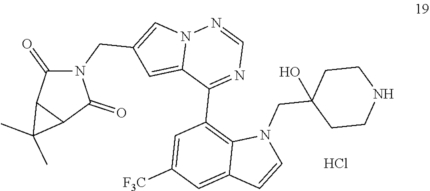 [SMILES: CC1(C)C2C(=O)N(Cc3cc4c(-c5cc(C(F)(F)F)cc6ccn(CC7(O)CCNCC7)c56)ncnn4c3)C(=O)C21 ; Cl]

### Step 1.

### Synthesis of tert-butyl 4-((7-bromo-5-(trifluoromethyl)-1H-indol-1-yl)methyl)-4-hydroxypiperidine-1-carboxylate (19a)

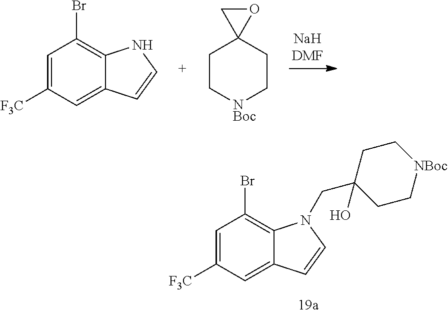 [SMILES: FC(F)(F)c1cc(Br)c2[nH]ccc2c1 ; CC(C)(C)OC(=O)N1CCC2(CC1)CO2 ; CC(C)(C)OC(=O)N1CCC(O)(Cn2ccc3cc(C(F)(F)F)cc(Br)c32)CC1]

To the solution of 7-bromo-5-(trifluoromethyl)-1H-indole (400 mg; 1.515 mmol) and tert-butyl 1-oxa-6-azaspiro[2.5]octane-6-carboxylate (420 mg; 1.970 mmol) in dry DMF (6 mL) NaH (60% in mineral oil; 95 mg; 3.940 mmol) was added at 0° C. and then the resulting mixture was heated at 70° C. for 1.5 hours. The reaction progress was monitored by LC-MS. When analysis indicated completion of the reaction, a whole was cooled to room temperature and concentrated in vacuo. The crude product was then purified by flash column chromatography on silica (hexane/AcOEt, 100:0 to 50:50, v/v, 27 min, 20 mL/min). Compound 19a was obtained in 68% yield (490 mg; 1.030 mmol).

ESI-MS m/z for C₁₆H₁₇BrF₃N₂O₃ found 420.7/422.8 [M+H-tBu]⁺; Rₜ=1.85 min (Method A); ¹H NMR (700 MHz, CDCl₃) δ 7.91-7.88 (m, 1H), 7.65-7.62 (m, 1H), 7.36-7.33 (m, 1H), 6.69-6.65 (m, 1H), 4.80-4.68 (m, 2H), 4.08-3.87 (m, 2H), 3.09-2.96 (m, 2H), 1.74 (s, 2H), 1.48 (s, 9H), 1.42-1.35 (m, 2H); ¹⁹F NMR (659 MHz, CDCl₃) δ −60.71.

### Step 2.

### Synthesis of (1-((1-(tert-butoxycarbonyl)-4-hydroxypiperidin-4-yl)methyl)-5-(trifluoromethyl)-1H-indol-7-yl)boronic acid (19b)

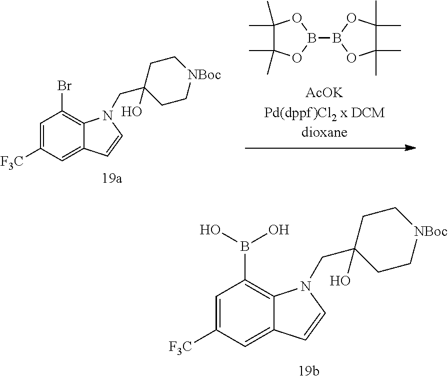

In a Schlenk flask was placed the compound 19a (50 mg; 0.105 mmol), bis(pinacolato)diboron (53 mg; 0.210 mmol), AcOK (31 mg; 0.315 mmol), Pd(dppf)Cl₂×DCM (9 mg; 0.011 mmol) and dioxane (1.5 mL). The reaction mixture was intensively flushed with Ar. The mixture was capped and stirred at 90° C. overnight. The reaction progress was monitored by LC-MS. When analysis indicated completion of the reaction, the reaction mixture was concentrated in vacuo and the crude product was used to the next step without any additional purification.

ESI-MS m/z for C₁₅H₁₉BF₃N₂O₃ found 343.0 [M+H-Boc]⁺; Rₜ=1.68 min (Method A)

### Step 3.

### Synthesis of tert-butyl 4-((7-(6-((6,6-dimethyl-2,4-dioxo-3-azabicyclo[3.1.0]hexan-3-yl)methyl)pyrrolo[2,1-f][1,2,4]triazin-4-yl)-5-(trifluoromethyl)-1H-indol-1-yl)methyl)-4-hydroxypiperidine-1-carboxylate (19c)

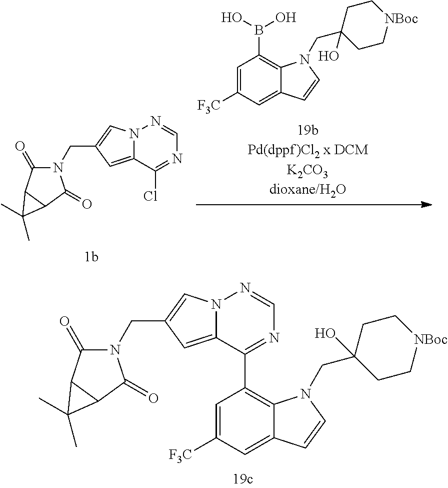 [SMILES: CC(C)(C)OC(=O)N1CCC(O)(Cn2ccc3cc(C(F)(F)F)cc(B(O)O)c32)CC1 ; CC1(C)C2C(=O)N(Cc3cc4c(Cl)ncnn4c3)C(=O)C21 ; CC(C)(C)OC(=O)N1CCC(O)(Cn2ccc3cc(C(F)(F)F)cc(-c4ncnn5cc(CN6C(=O)C7C(C6=O)C7(C)C)cc45)c32)CC1]

The title compound (19c) was obtained from 1b (48 mg; 0.155 mmol) and from 19b (the crude product) according to the General Procedure IV in 17% yield (per two steps)(12 mg; 0.018 mmol). The crude product was purified by flash column chromatography on silica (hexane/AcOEt, 100:0 to 0:100, v/v, 27 min, 10 mL/min).

ESI-MS m/z for C₃₄H₃₈F₃N₆O₅ found 667.4 [M+H]⁺; Rₜ=1.79 min (Method A)

### Step 4.

### Synthesis of 3-((4-(1-((4-hydroxypiperidin-4-yl)methyl)-5-(trifluoromethyl)-1H-indol-7-yl)pyrrolo[2,1-f][1,2,4]triazin-6-yl)methyl)-6,6-dimethyl-3-azabicyclo[3.1.0]hexane-2,4-dione hydrochloride (19)

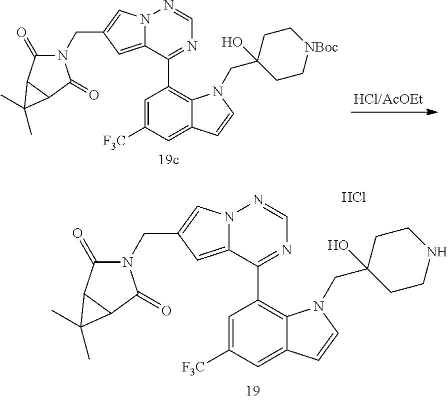 [SMILES: CC(C)(C)OC(=O)N1CCC(O)(Cn2ccc3cc(C(F)(F)F)cc(-c4ncnn5cc(CN6C(=O)C7C(C6=O)C7(C)C)cc45)c32)CC1 ; Cl ; CC1(C)C2C(=O)N(Cc3cc4c(-c5cc(C(F)(F)F)cc6ccn(CC7(O)CCNCC7)c56)ncnn4c3)C(=O)C21]

The title compound was obtained as a white solid as a hydrochloride salt from 19c (29 mg; 0.043 mmol) according to the General Procedure IIIa in 35% yield (9 mg; 0.015 mmol). The crude product was purified by preparative reversed-phase column chromatography (column: Cosmosil Cholester 20×250 mm, water+0.3‰ HCl (36%)/MeCN, 95:5 to 30:70, 30 min, 20 mL/min, Rₜ=17.5 min).

ESI-MS m/z for C₂₉H₃₀F₃N₆O₃ found 567.6 [M+H]⁺; Rₜ=1.12 min (Method A); ¹H NMR (700 MHz, D₂O) δ 8.48 (s, 1H), 8.19-8.14 (m, 1H), 8.05-8.03 (m, 1H), 7.55-7.49 (m, 1H), 7.47-7.39 (m, 1H), 6.87-6.82 (m, 1H), 6.61-6.57 (m, 1H), 4.58-4.49 (m, 2H), 4.00-3.91 (m, 2H), 3.07-2.97 (m, 2H), 2.94-2.85 (m, 2H), 2.51 (s, 2H), 1.39-1.30 (m, 2H), 1.24-1.17 (m, 2H), 1.15 (s, 3H), 0.92 (s, 3H); ¹⁹F NMR (659 MHz, D₂O) δ −60.37.

### Example 20

### Synthesis of 3-((4-(5-chloro-3-methyl-2-(((3S,5S)-5-methylpyrrolidin-3-yl)oxy)phenyl)pyrrolo[2,1-f][1,2,4]triazin-6-yl)methyl)-6,6-dimethyl-3-azabicyclo[3.1.0]hexane-2,4-dione hydrochloride (20)

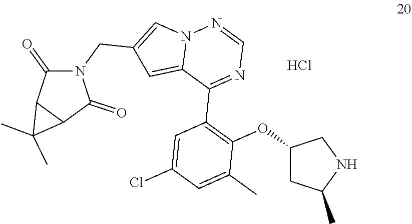 [SMILES: Cc1cc(Cl)cc(-c2ncnn3cc(CN4C(=O)C5C(C4=O)C5(C)C)cc23)c1O[C@@H]1CN[C@@H](C)C1 ; Cl]

### Step 1.

### Synthesis of tert-butyl (2S,4S)-4-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenoxy)-2-methylpyrrolidine-1-carboxylate (20a)

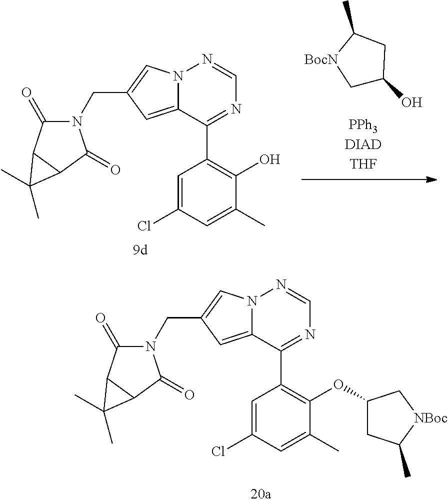 [SMILES: Cc1cc(Cl)cc(-c2ncnn3cc(CN4C(=O)C5C(C4=O)C5(C)C)cc23)c1O ; C[C@H]1C[C@@H](O)CN1C(=O)OC(C)(C)C ; Cc1cc(Cl)cc(-c2ncnn3cc(CN4C(=O)C5C(C4=O)C5(C)C)cc23)c1O[C@H]1C[C@H](C)N(C(=O)OC(C)(C)C)C1]

To a cooled to −10° C. solution of 9d (42 mg; 0.102 mmol), tert-butyl (2S,4R)-4-hydroxy-2-methylpyrrolidine-1-carboxylate (25 mg; 0.123 mmol) and PPh₃ (32 mg; 0.123 mmol) in THE (0.7 mL) DIAD (26 μL; 0.133 mmol) was slowly added. The resulting mixture was stirred at room temperature overnight. The reaction progress was monitored by LC-MS. Then next portion of tert-butyl (2S,4R)-4-hydroxy-2-methylpyrrolidine-1-carboxylate (12 mg; 0.061 mmol), PPh₃ (16 mg; 0.061 mmol), DIAD (13 μL; 0.067 mmol) and THE (0.5 mL) was added and the resulting mixture was stirred at room temperature for 2 days. When analysis indicated completion of the reaction, the solvent was evaporated in vacuo and the residue was purified by flash column chromatography on silica (hexane/AcOEt, 100:0 to 0:100, v/v, 20 min). Compound 20a was obtained in 99% yield (60 mg; 0.101 mmol).

ESI-MS m/z for C₃₁H₃₇ClN₅O₅ found 594.0/596.0 [M+H]⁺; Rₜ=1.92 min (Method A)

### Step 2.

### Synthesis of 3-((4-(5-chloro-3-methyl-2-(((3S,5S)-5-methylpyrrolidin-3-yl)oxy)phenyl)pyrrolo[2,1-f][1,2,4]triazin-6-yl)methyl)-6,6-dimethyl-3-azabicyclo[3.1.0]hexane-2,4-dione hydrochloride (20)

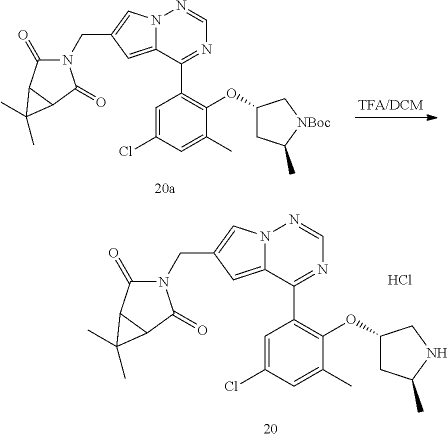 [SMILES: Cc1cc(Cl)cc(-c2ncnn3cc(CN4C(=O)C5C(C4=O)C5(C)C)cc23)c1O[C@H]1C[C@H](C)N(C(=O)OC(C)(C)C)C1 ; Cc1cc(Cl)cc(-c2ncnn3cc(CN4C(=O)C5C(C4=O)C5(C)C)cc23)c1O[C@@H]1CN[C@@H](C)C1 ; Cl]

The title compound (20) was obtained as a hydrochloride salt from 20a (60 mg; 0.101 mmol) according to the General Procedure IIIb in 52% yield (28 mg; 0.053 mmol). The crude product was purified by preparative reversed-phase column chromatography (C-18, water+0.3‰ HCl (36%)/MeCN, 99:1 to 45:55, 30 min, 20 mL/min, Rₜ=23.8 min).

ESI-MS m/z for C₂₆H₂₉ClN₅O₃ found 494.5/496.5 [M+H]⁺; Rₜ=1.10 min (Method A); ¹H NMR (700 MHz, D₂O) δ 8.45 (s, 1H), 8.05-8.00 (m, 1H), 7.51-7.48 (m, 1H), 7.30-7.25 (m, 1H), 6.78-6.73 (m, 1H), 4.66-4.62 (m, 2H), 4.41-4.38 (m, 1H), 3.32-3.28 (m, 1H), 3.28-3.22 (m, 1H), 3.19-3.14 (m, 1H), 2.55 (s, 2H), 2.31 (s, 3H), 1.86-1.79 (m, 1H), 1.43-1.35 (m, 1H), 1.17 (s, 3H), 1.12 (d, J=6.7 Hz, 3H), 0.97 (s, 3H).

### Example 21

### Synthesis of 3-((4-(5-chloro-1-((4-fluoropiperidin-4-yl)methyl)-1H-indol-7-yl)-5-methylpyrrolo[2,1-f][1,2,4]triazin-6-yl)methyl)-6,6-dimethyl-3-azabicyclo[3.1.0]hexane-2,4-dione hydrochloride (21)

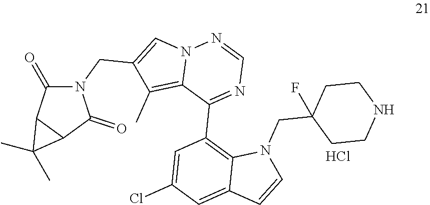 [SMILES: Cc1c(CN2C(=O)C3C(C2=O)C3(C)C)cn2ncnc(-c3cc(Cl)cc4ccn(CC5(F)CCNCC5)c34)c12 ; Cl]

### Step 1.

### Synthesis of tert-butyl 4-((5-chloro-7-(6-((6,6-dimethyl-2,4-dioxo-3-azabicyclo[3.1.0]hexan-3-yl)methyl)-5-methylpyrrolo[2,1-f][1,2,4]triazin-4-yl)-1H-indol-1-yl)methyl)-4-fluoropiperidine-1-carboxylate (21a)

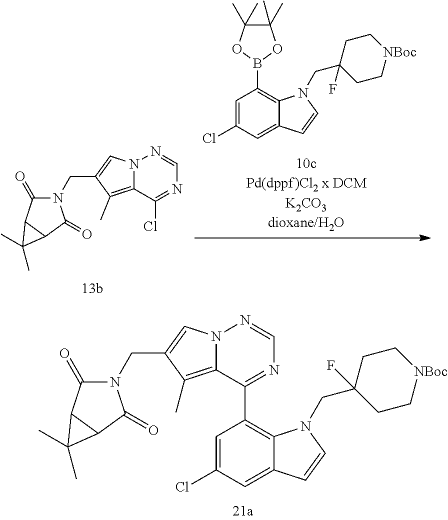 [SMILES: CC(C)(C)OC(=O)N1CCC(F)(Cn2ccc3cc(Cl)cc(B4OC(C)(C)C(C)(C)O4)c32)CC1 ; Cc1c(CN2C(=O)C3C(C2=O)C3(C)C)cn2ncnc(Cl)c12 ; Cc1c(CN2C(=O)C3C(C2=O)C3(C)C)cn2ncnc(-c3cc(Cl)cc4ccn(CC5(F)CCN(C(=O)OC(C)(C)C)CC5)c34)c12]

The title compound (21a) was obtained from 13b (42 mg; 0.134 mmol) and from 10c (33 mg; 0.067 mmol) according to the General Procedure IV in 69% yield (30 mg; 0.046 mmol). The crude product was purified by flash column chromatography on silica (hexane/AcOEt, 100:0 to 0:100, v/v, 22 min, 10 mL/min).

ESI-MS m/z for C₃₄H₃₉ClFN₆O₄ found 649.1/651.1 [M+H]⁺; Rₜ=1.97 min (Method A)

### Step 2.

### Synthesis of 3-((4-(5-chloro-1-((4-fluoropiperidin-4-yl)methyl)-1H-indol-7-yl)-5-methylpyrrolo[2,1-f][1,2,4]triazin-6-yl)methyl)-6,6-dimethyl-3-azabicyclo[3.1.0]hexane-2,4-dione hydrochloride (21)

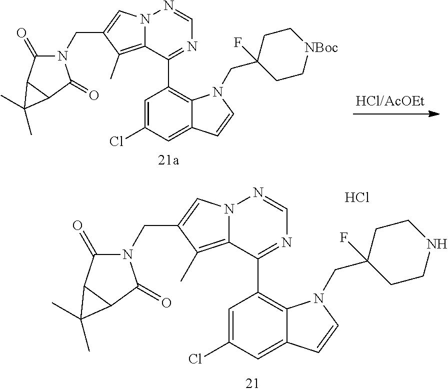 [SMILES: Cc1c(CN2C(=O)C3C(C2=O)C3(C)C)cn2ncnc(-c3cc(Cl)cc4ccn(CC5(F)CCN(C(=O)OC(C)(C)C)CC5)c34)c12 ; Cc1c(CN2C(=O)C3C(C2=O)C3(C)C)cn2ncnc(-c3cc(Cl)cc4ccn(CC5(F)CCNCC5)c34)c12 ; Cl]

The title compound (21) was obtained as a hydrochloride salt as a mixture of rotamers from 21a (28 mg; 0.043 mmol) according to the General Procedure IIIa in 19% yield (5 mg; 0.008 mmol). The crude product was purified by preparative reversed-phase column chromatography (C-18, water+0.3‰ HCl (36%)/MeCN, 95:5 to 20:80, 30 min, 20 mL/min, Rₜ=17.6 min).

ESI-MS m/z for C₂₉H₃₁ClFN₆O₂ found 549.4/551.4 [M+H]⁺; Rₜ=1.19 min (Method A); ¹H NMR (700 MHz, Methanol-d₄) δ 8.51 (s, 1H), 8.15 (s, 1H), 7.89-7.86 (m, 1H), 7.42-7.38 (m, 1H), 7.24-7.21 (m, 1H), 6.77-6.74 (m, 1H), 4.71-4.64 (m, 1H), 4.64-4.56 (m, 1H), 4.37-4.29 (m, 1H), 3.95-3.83 (m, 1H), 3.29-3.15 (m, 2H), 2.98-2.89 (m, 2H), 2.50-2.43 (m, 2H), 1.93-1.78 (m, 1H), 1.73 (s, 3H), 1.66-1.49 (m, 2H), 1.42-1.30 (m, 1H), 1.25 (s, 3H), 1.06 (s, 3H).

### Example 22

### Synthesis of 3-((4-(1-((4-fluoropiperidin-4-yl)methyl)-5-(trifluoromethyl)-1H-indol-7-yl)pyrrolo[2,1-f][1,2,4]triazin-6-yl)methyl)-6,6-dimethyl-3-azabicyclo[3.1.0]hexane-2,4-dione hydrochloride (22)

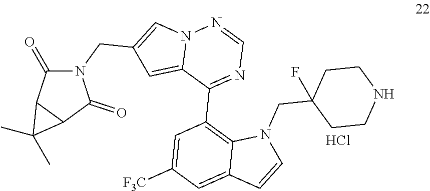 [SMILES: CC1(C)C2C(=O)N(Cc3cc4c(-c5cc(C(F)(F)F)cc6ccn(CC7(F)CCNCC7)c56)ncnn4c3)C(=O)C21 ; Cl]

The title compound (22) was obtained as a hydrochloride salt in 4% overall yield in a similar way to Example 10 with the exception that, in the first step of the synthesis, 7-bromo-5-(trifluoromethyl)-1H-indole was used instead of 7-bromo-5-chloro-1H-indole. The crude product was purified by preparative reversed-phase column chromatography (C-18, water+0.3‰ HCl (36%)/MeCN, 95:5 to 20:80, 30 min, 20 mL/min, Rₜ=17.6 min).

ESI-MS m/z for C₂₉H₂₉F₄N₆O₂ found 569.5 [M+H]⁺; Rₜ=1.20 min (Method A); ¹H NMR (700 MHz, Methanol-d₄) δ 8.66 (s, 1H), 8.24-8.21 (m, 1H), 8.16-8.13 (m, 1H), 7.73-7.69 (m, 1H), 7.54-7.49 (m, 1H), 6.95-6.92 (m, 1H), 6.77-6.74 (m, 1H), 4.70 (s, 2H), 4.54-4.47 (m, 2H), 3.21-3.14 (m, 2H), 2.91-2.82 (m, 2H), 2.48 (s, 2H), 1.82-1.69 (m, 2H), 1.49-1.42 (m, 2H), 1.25 (s, 3H), 1.06 (s, 3H).

### Example 23

### Synthesis of 3-((4-(2-((3S,4S)-3-amino-4-methylpyrrolidine-1-carbonyl)-5-chloro-3-methylphenyl)pyrrolo[2,1-f][1,2,4]triazin-6-yl)methyl)-6,6-dimethyl-3-azabicyclo[3.1.0]hexane-2,4-dione 2,2,2-trifluoroacetate (23)

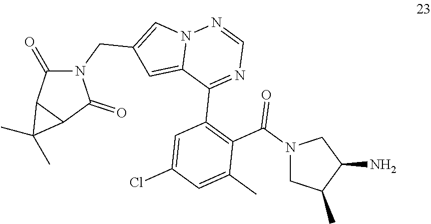 [SMILES: Cc1cc(Cl)cc(-c2ncnn3cc(CN4C(=O)C5C(C4=O)C5(C)C)cc23)c1C(=O)N1C[C@@H](N)[C@@H](C)C1]

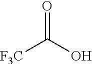

### Step 1.

### Synthesis of tert-butyl ((3S,4S)-1-(4-chloro-2-iodo-6-methylbenzoyl)-4-methylpyrrolidin-3-yl)carbamate (23a)

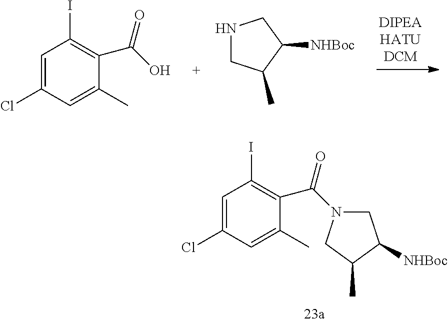 [SMILES: Cc1cc(Cl)cc(I)c1C(=O)O ; C[C@H]1CNC[C@H]1NC(=O)OC(C)(C)C ; Cc1cc(Cl)cc(I)c1C(=O)N1C[C@H](C)[C@H](NC(=O)OC(C)(C)C)C1]

The title compound (23a) was obtained from 4-chloro-2-iodo-6-methylbenzoic acid (237 mg; 0.799 mmol) and from tert-butyl ((3S,4S)-4-methylpyrrolidin-3-yl)carbamate (160 mg; 0.799 mmol) according to the General Procedure I in 53% yield (202 mg; 0.422 mmol). The crude product was purified by flash column chromatography on silica (hexane/AcOEt, 100:0 to 0:100, v/v, 22 min, 15 mL/min).

ESI-MS m/z for C18H₂₅ClIN₂O₃ found 478.9/480.9 [M+H]⁺; Rₜ=1.60 min (Method A)

### Step 2.

### Synthesis of tert-butyl ((3S,4S)-1-(4-chloro-2-methyl-6-(4,4,5,5-tetramethyl-1,3,2-dioxaborolan-2-yl)benzoyl)-4-methylpyrrolidin-3-yl)carbamate (23b)

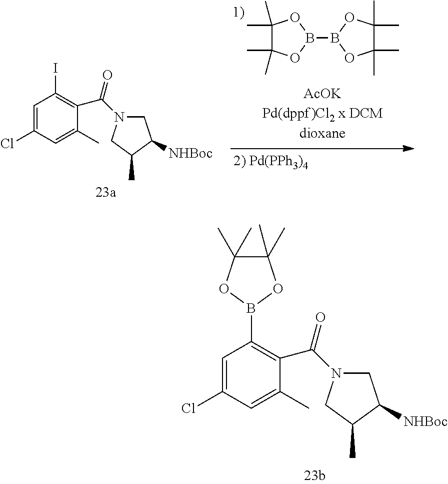

In a Schlenk flask was placed the compound 23a (115 mg; 0.241 mmol), bis(pinacolato)diboron (122 mg; 0.481 mmol), AcOK (71 mg; 0.723 mmol) and Pd(dppf)Cl₂×DCM (20 mg; 0.024 mmol) and dioxane (7.5 mL). The reaction mixture was intensively flushed with Ar. The mixture was capped and stirred at 90° C. overnight. Then Pd(PPh₃)₄ (14 mg; 0.012 mmol) was added and stirred at 90° C. overnight. Then another portion of bis(pinacolato)diboron (61 mg; 0.240 mmol), AcOK (35 mg; 361 mmol) and Pd(dppf)Cl₂×DCM (19 mg; 0.024 mmol) was added and stirred at 90° C. overnight and after that another portion of Pd(dppf)Cl₂×DCM (19 mg; 0.024 mmol) was added and stirred at 90° C. for 2 days. The reaction progress was monitored by LC-MS. When analysis indicated completion of the reaction, the reaction mixture was concentrated in vacuo and the residue was purified by flash column chromatography on silica (hexane/AcOEt, 100:0 to 0:100, v/v, 22 min, 10 mL/min) and then was used to the next step without any additional purification.

ESI-MS m/z for C₂₄H₃₇BClN₂O₅ found 479.1/481.0 [M+H]⁺; Rₜ=1.83 min (Method A)

### Step 3.

### Synthesis of tert-butyl ((3S,4S)-1-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylbenzoyl)-4-methylpyrrolidin-3-yl)carbamate (23c)

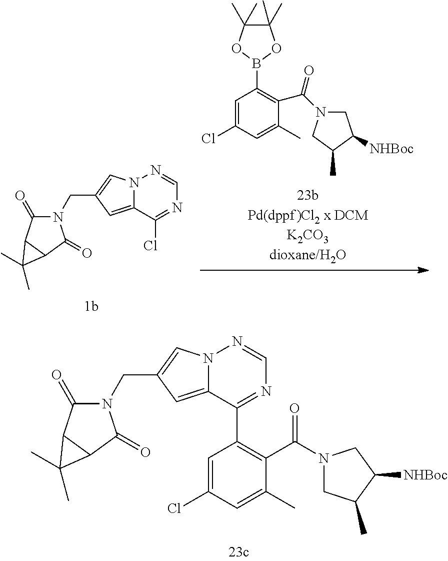 [SMILES: CC1(C)C2C(=O)N(Cc3cc4c(Cl)ncnn4c3)C(=O)C21 ; Cc1cc(Cl)cc(B2OC(C)(C)C(C)(C)O2)c1C(=O)N1C[C@H](C)[C@H](NC(=O)OC(C)(C)C)C1 ; Cc1cc(Cl)cc(-c2ncnn3cc(CN4C(=O)C5C(C4=O)C5(C)C)cc23)c1C(=O)N1C[C@H](C)[C@H](NC(=O)OC(C)(C)C)C1]

The title compound (23a) was obtained as a mixture of rotamers from 1b (24 mg; 0.077 mmol) and from 23b (the crude product) according to the General Procedure IV in 14% yield (per two steps)(22 mg; 0.035 mmol). The crude product was purified by flash column chromatography on silica (hexane/AcOEt, 100:0 to 0:100, v/v, 22 min, 14 mL/min).

ESI-MS m/z for C₃₂H₃₈ClN₆O₅ found 621.1/623.1 [M+H]⁺; Rₜ=1.72 min (Method A)

### Step 4.

### Synthesis of 3-((4-(2-((3S,4S)-3-amino-4-methylpyrrolidine-1-carbonyl)-5-chloro-3-methylphenyl)pyrrolo[2,1-f][1,2,4]triazin-6-yl)methyl)-6,6-dimethyl-3-azabicyclo[3.1.0]hexane-2,4-dione 2,2,2-trifluoroacetate (23)

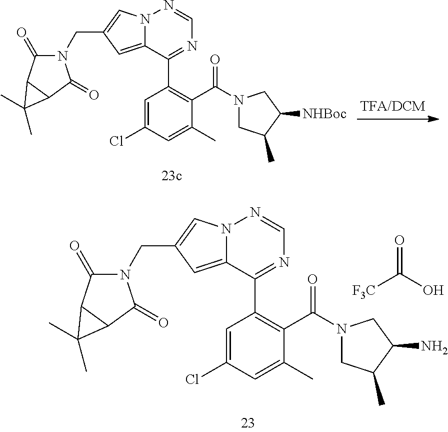

The title compound (23) was obtained as a TFA salt from 23c (22 mg; 0.035 mmol) according to the General Procedure IIIb in 23% yield (5 mg; 0.008 mmol). The crude product was purified by preparative reversed-phase column chromatography (C-18, water/MeCN+1‰ TFA, 98:2 to 30:70, 20 min, 20 mL/min, Rₜ=13.2 min).

ESI-MS m/z for C₂₇H₃₀ClN₆O₃ found 521.4/523.4 [M+H]⁺; Rₜ=1.05 min (Method A); ¹H NMR (700 MHz, D20, 333 K) δ 8.81-8.74 (m, 1H), 8.41-8.34 (m, 1H), 8.05-7.88 (m, 2H), 7.25-7.19 (m, 1H), 5.08-5.00 (m, 2H), 4.40-4.09 (m, 2H), 4.04-3.91 (m, 1H), 3.75-3.57 (m, 2H), 3.54-3.33 (m, 1H), 3.20-3.05 (m, 1H), 2.95-2.63 (m, 4H), 1.56-0.74 (m, 9H).

### Example 24

### Synthesis of 3-((4-(5-chloro-1-(morpholin-2-ylmethyl)-1H-indol-7-yl)pyrrolo[2,1-f][1,2,4]triazin-6-yl)methyl)-6,6-dimethyl-3-azabicyclo[3.1.0]hexane-2,4-dione dihydrochloride (24)

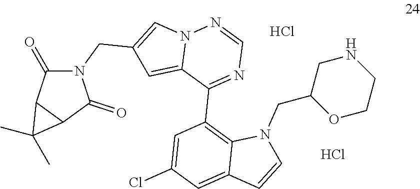 [SMILES: CC1(C)C2C(=O)N(Cc3cc4c(-c5cc(Cl)cc6ccn(CC7CNCCO7)c56)ncnn4c3)C(=O)C21 ; Cl ; Cl]

The title compound (24) was obtained as a dihydrochloride salt in 14% overall yield in a similar way to Example 5 with the exception that, in the first step of the synthesis, 7-bromo-5-chloro-1H-indole and tert-butyl 2-(bromomethyl)morpholine-4-carboxylate were used instead of 7-bromo-5-(trifluoromethyl)-1H-indole and tert-butyl 3-((methylsulfonyl)oxy)pyrrolidine-1-carboxylate and to this reaction 0.36 equivalents of KI was added, and in the fourth step of the synthesis, the General Procedure IIIa was used instead of the General Procedure IIIb. The crude product was purified by preparative reversed-phase column chromatography (column: Cosmosil Cholester 20×250 mm, water+0.3‰ HCl (36%)/MeCN, 95:5 to 20:80, 30 min, 20 mL/min, Rₜ=15.5 min).

ESI-MS m/z for C₂₇H₂₈ClN₆O₃ found 519.4/521.4 [M+H]⁺; Rₜ=1.11 min (Method A); ¹H NMR (700 MHz, Methanol-d₄) δ 8.59 (s, 1H), 8.11-8.07 (m, 1H), 7.87-7.82 (m, 1H), 7.42-7.39 (m, 1H), 7.37-7.32 (m, 1H), 6.78-6.74 (m, 1H), 6.72-6.67 (m, 1H), 4.70 (s, 2H), 4.27-4.13 (m, 2H), 3.71-3.63 (m, 1H), 3.50-3.44 (m, 1H), 3.26-3.19 (m, 1H), 3.08-2.95 (m, 2H), 2.78-2.67 (m, 1H), 2.53-2.45 (m, 3H), 1.26 (s, 3H), 1.12 (s, 3H).

### Example 25

### Synthesis of 4-((5-chloro-7-(6-((6,6-dimethyl-2,4-dioxo-3-azabicyclo[3.1.0]hexan-3-yl)methyl)pyrrolo[2,1-f][1,2,4]triazin-4-yl)-1H-indol-1-yl)methyl)piperidine-4-carbonitrile 2,2,2-trifluoroacetate (25)

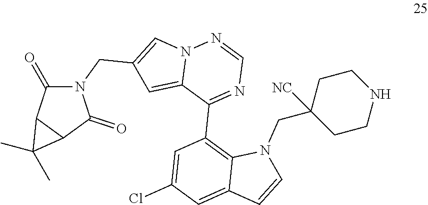 [SMILES: CC1(C)C2C(=O)N(Cc3cc4c(-c5cc(Cl)cc6ccn(CC7(C#N)CCNCC7)c56)ncnn4c3)C(=O)C21]

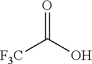

The title compound (25) was obtained as a TFA salt in 4% overall yield in a similar way to Example 5 with the exception that, in the first step of the synthesis, 7-bromo-5-chloro-1H-indole and tert-butyl 4-cyano-4-((tosyloxy)methyl)piperidine-1-carboxylate were used instead of 7-bromo-5-(trifluoromethyl)-1H-indole and tert-butyl 3-((methylsulfonyl)oxy)pyrrolidine-1-carboxylate and to this reaction 0.35 equivalents of KI was added, and in the fourth step of the synthesis, the General Procedure IIIa was used instead of the General Procedure IIIb. The crude product was purified by preparative reversed-phase column chromatography (C-18, water+0.4‰ TFA/MeCN, 98:2 to 30:70, 30 min, 20 mL/min, Rₜ=21.5 min).

ESI-MS m/z for C₂₉H₂₉ClN₇O₂ found 542.4/544.4 [M+H]⁺; Rₜ=1.19 min (Method A); ¹H NMR (700 MHz, Methanol-d₄) δ 8.61 (s, 1H), 8.09-8.03 (m, 1H), 7.88-7.84 (m, 1H), 7.54-7.51 (m, 1H), 7.48-7.45 (m, 1H), 6.80-6.76 (m, 1H), 6.73-6.70 (m, 1H), 4.67 (s, 2H), 4.50 (s, 2H), 3.00-2.91 (m, 2H), 2.45 (s, 2H), 1.76-1.69 (m, 2H), 1.53-1.45 (m, 2H), 1.36-1.27 (m, 2H), 1.23 (s, 3H), 1.07 (s, 3H).

### Example 26

### Synthesis of 3-((4-(5-chloro-1-((5,5-difluoropiperidin-3-yl)methyl)-1H-indol-7-yl)pyrrolo[2,1-f][1,2,4]triazin-6-yl)methyl)-6,6-dimethyl-3-azabicyclo[3.1.0]hexane-2,4-dione dihydrochloride (26)

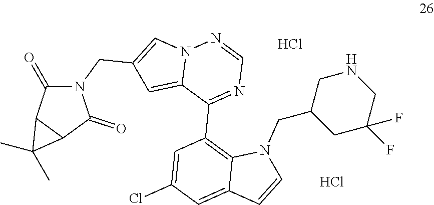 [SMILES: CC1(C)C2C(=O)N(Cc3cc4c(-c5cc(Cl)cc6ccn(CC7CNCC(F)(F)C7)c56)ncnn4c3)C(=O)C21 ; Cl ; Cl]

The title compound (26) was obtained as a dihydrochloride salt in 13% overall yield in a similar way to Example 5 with the exception that, in the first step of the synthesis, 7-bromo-5-chloro-1H-indole and tert-butyl 3,3-difluoro-5-(((methylsulfonyl)oxy)methyl)piperidine-1-carboxylate 26a (the synthesis of this compound is described below) were used instead of 7-bromo-5-(trifluoromethyl)-1H-indole and tert-butyl 3-((methylsulfonyl)oxy)pyrrolidine-1-carboxylate and to this reaction 0.36 equivalents of KI was added, and in the fourth step of the synthesis, the General Procedure IIIa was used instead of the General Procedure IIIb. The crude product was purified by preparative reversed-phase column chromatography (C-18, water+0.3‰ HCl (36%)/MeCN, 99:1 to 30:70, 30 min, 20 mL/min, Rₜ=19.25 min).

ESI-MS m/z for C₂₈H₂₈ClF₂N₆O₂ found 553.5/555.5 [M+H]⁺; Rₜ=1.19 min (Method A); ¹H NMR (700 MHz, D₂O) δ 8.50 (s, 1H), 8.08-8.06 (m, 1H), 7.78-7.76 (m, 1H), 7.27-7.25 (m, 1H), 7.17-7.14 (m, 1H), 6.64-6.62 (m, 1H), 6.60 (s, 1H), 4.55-4.50 (m, 2H), 3.83-3.79 (m, 1H), 3.77-3.69 (m, 1H), 3.47-3.44 (m, 1H), 3.14-3.08 (m, 1H), 2.70-2.67 (m, 1H), 2.61-2.57 (m, 1H), 2.44 (s, 2H), 1.88-1.83 (m, 1H), 1.50-1.44 (m, 1H), 1.27-1.23 (m, 1H), 1.13 (s, 3H), 0.95 (s, 3H).

### Synthesis of tert-butyl 3,3-difluoro-5-(((methylsulfonyl)oxy)methyl)piperidine-1-carboxylate (26a)

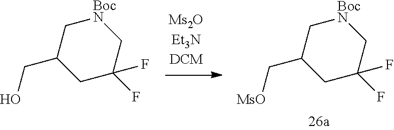 [SMILES: CC(C)(C)OC(=O)N1CC(CO)CC(F)(F)C1 ; CC(C)(C)OC(=O)N1CC(COS(C)(=O)=O)CC(F)(F)C1]

The title compound (26a) was obtained from tert-butyl 3,3-difluoro-5-(hydroxymethyl)piperidine-1-carboxylate (443 mg; 1.76 mmol) according to the General Procedure V and after standard work-up the crude product 26a (99% yield; 575 mg; 1.75 mmol) was taken to the next step.

ESI-MS m/z for C₁₂H₂₁F₂NO₅SNa found 352.0 [M+Na]⁺; Rₜ=1.33 min (Method A); ¹H NMR (700 MHz, CDCl₃) δ 4.24-4.14 (m, 2H), 4.06-3.80 (m, 2H), 3.39-3.28 (m, 1H), 3.14-2.98 (m, 4H), 2.42-2.30 (m, 1H), 2.30-2.20 (m, 1H), 1.92-1.82 (m, 1H), 1.49 (s, 9H).

### Example 27

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione dihydrochloride (27)

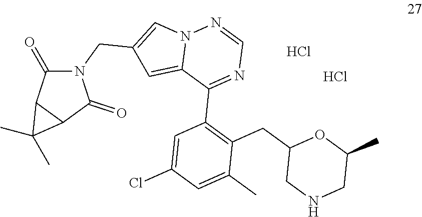

### Step 1.

### Synthesis of (S)-1-((4-methoxybenzyl)amino)propan-2-ol (27a)

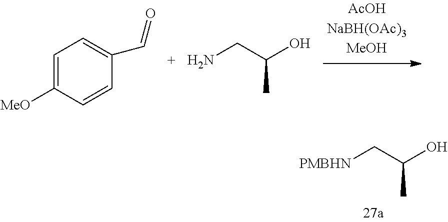

To the solution of (S)-1-aminopropan-2-ol (2.00 g; 26.60 mmol) in MeOH (20 mL) 4-methoxybenzaldehyde (1.10 mL; 8.98 mmol) and glacial acetic acid (AcOH) (1.06 mL) were added and the mixture was stirred for 40 minutes. Then sodium triacetoxyborohydride (NaBH(OAc)₃; 2.09 g; 9.88 mmol) was then added in one portion and this mixture was stirred at room temperature overnight. After this time a 0.5 M aqueous solution of NaOH was added and the biphasic mixture was stirred for 30 minutes. The layers were separated and the aqueous layer was additionally extracted with DCM (4×). The combined organic extracts were dried over anhydrous MgSO₄, filtered and the solvents were evaporated in vacuo. The crude product was purified by flash column chromatography on silica (DCM/MeOH, 100:0 to 0:100, v/v, 12 min). Compound 27a was obtained in 84% yield (16.35 g; 8.38 mmol).

ESI-MS m/z for C₁₁H₁₈NO₂ found 196.1 [M+H]⁺; Rₜ=0.90 min (Method B); ¹H NMR (700 MHz, CDCl₃) δ 7.31-7.28 (m, 2H), 6.92-6.88 (m, 2H), 3.89-3.86 (m, 1H), 3.85-3.77 (m, 5H), 2.78 (dd, J=12.1, 2.9 Hz, 1H), 2.50 (dd, J=12.1, 9.5 Hz, 1H), 1.17 (d, J=6.3 Hz, 3H).

### Step 2.

### Synthesis of (S)-4-(4-methoxybenzyl)-6-methylmorpholin-3-one (27b)

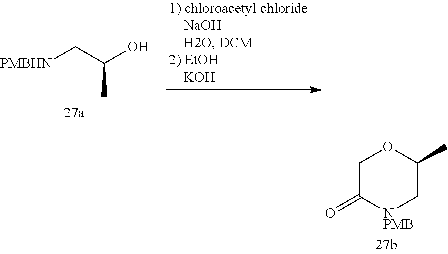

To the solution of 27a (1.2 g; 6.14 mmol) in DCM (8 mL) a solution of NaOH (245 mg; 6.14 mmol) in H₂O (5 mL) was added and the mixture was cooled to 0° C. Then to this solution chloroacetyl chloride (0.49 mL; 6.14 mmol) in DCM (2 mL) was added dropwise and the reaction mixture was then stirred at room temperature overnight. After this time, the phases were separated and an organic one was washed with 1 M NaOH, 1 M HCl and brine and then was dried over anhydrous MgSO₄, filtered and concentrated in vacuo. The residue was dissolved in EtOH (17 mL) and to this solution KOH (344 mg; 6.14 mmol) was added. The reaction mixture was stirred at room temperature overnight. Then the crude product was purified by flash column chromatography on silica (hexane/AcOEt, 100:0 to 0:100, v/v, 12 min). Compound 27b was obtained in 76% yield (1.1 g; 4.68 mmol).

ESI-MS m/z for C₁₃H₁₈NO₃ found 236.1 [M+H]⁺; Rₜ=0.97 min (Method A); ¹H NMR (700 MHz, CDCl₃) δ 7.24-7.20 (m, 2H), 6.91-6.87 (m, 2H), 4.68 (d, J=14.4 Hz, 1H), 4.44 (d, J=14.4 Hz, 1H), 4.34 (d, J=16.6 Hz, 1H), 4.23 (d, J=16.6 Hz, 1H), 3.87-3.81 (m, 4H), 3.16-3.05 (m, 2H), 1.23 (d, J=6.2 Hz, 3H).

### Step 3.

### Synthesis of (6S)-2-(4-chloro-2-iodo-6-methylbenzyl)-4-(4-methoxybenzyl)-6-methylmorpholin-3-one (27c)

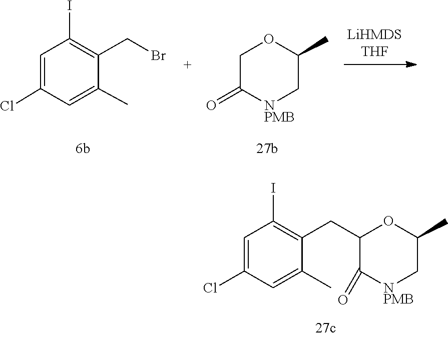

To the cooled to −78° C. solution of 27b (170 mg; 0.723 mmol) in THF (10 mL) a solution of LiHMDS (1 M in THF; 867 μL; 0.867 mmol) was added and the mixture was stirred at this temperature for 1 hour. Then to this mixture a solution of 6b (250 mg; 0.723 mmol) in THE (2 mL) was added and a whole was allowed to warm to room temperature over 3 hours. After this time, the reaction mixture was quenched with NH₄Cl and extracted with AcOEt (2×). The combined organic solutions were dried over anhydrous MgSO₄, filtered and concentrated in vacuo. Then the crude product was purified by flash column chromatography on silica (hexane/AcOEt, 100:0 to 50:50, v/v, 8 min). Compound 27c was obtained in 39% yield (140 mg; 0.280 mmol).

ESI-MS m/z for C₂₁H₂₄ClINO₃ found 500.1/502.1 [M+H]⁺; Rₜ=2.00 min (Method A); ¹H NMR (700 MHz, CDCl₃) δ 7.74-7.71 (m, 1H), 7.27-7.23 (m, 2H), 7.16-7.14 (m, 1H), 6.92-6.89 (m, 2H), 4.77-4.72 (m, 1H), 4.44-4.36 (m, 2H), 3.83 (s, 3H), 3.74-3.66 (m, 2H), 3.20-3.14 (m, 2H), 3.09-3.04 (m, 1H), 2.43 (s, 3H), 1.11 (d, J=6.1 Hz, 3H).

### Step 4.

### Synthesis of (6S)-2-(4-chloro-2-iodo-6-methylbenzyl)-4-(4-methoxybenzyl)-6-methylmorpholine (27d)

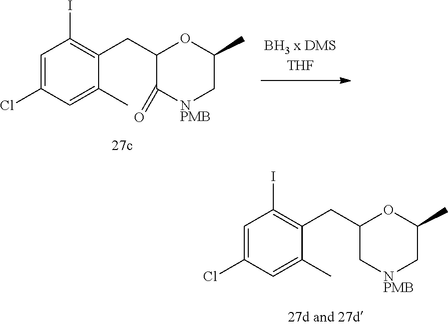

To the cooled to 0° C. solution of 27c (2.85 g; 5.70 mmol) in THE (28.5 mL) BH₃×DMS (1.62 mL; 17.10 mmol) was added dropwise under an argon atmosphere and this mixture was then warmed to 70° C. and stirred at this temperature overnight. The reaction progress was monitored by LC-MS. When analysis indicated completion of the reaction, to this mixture (cooled to 0° C.) 2 M HCl was added and whole was stirred at 70° C. for 1 hour. Then the reaction mixture was cooled to room temperature and to this solution 4 M NaOH was added to pH 8 and extracted with DCM (3×). The combined organic solutions were then dried over anhydrous Na₂SO₄, filtered and concentrated in vacuo. The crude product was purified by flash column chromatography on silica (hexane/AcOEt, 100:0 to 0:100, v/v, 20 min, 21 mL/min). Compounds 27d and 27d′ were obtained as separated diastereoisomers in 45% yield (for 27d)(1.24 g; 2.56 mmol) and in 35% yield (for 27d′) (964 mg; 1.99 mmol).

For 27d: ESI-MS m/z for C₂₁H₂₆ClINO₂ found 486.2/488.2 [M+H]⁺; Rₜ=6.02 min (Method E);

For 27d′: ESI-MS m/z for C₂₁H₂₆ClINO₂ found 486.1/488.1 [M+H]⁺; Rₜ=6.27 min (Method E)

### Step 5.

### Synthesis of (6S)-2-(4-chloro-2-iodo-6-methylbenzyl)-6-methylmorpholine (27e)

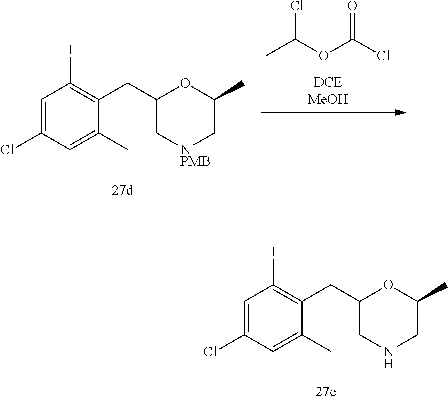

To the solution of 27d (136 mg; 0.28 mmol) in DCE (2 mL) 1-chloroethyl carbonochloridate (0.10 mL; 0.93 mmol) was added and this mixture was then stirred at 80° C. overnight. The reaction progress was monitored by LC-MS. When analysis indicated completion of the reaction, the solvent was evaporated in vacuo and the residue was dissolved in MeOH and whole was stirred at 80° C. for 1 hour. Then MeOH was evaporated in vacuo to give the crude product which was used to the next step without any additional purification.

ESI-MS m/z for C₁₃H₁₈ClINO found 366.1/368.1 [M+H]⁺; Rₜ=1.02 min (Method A)

### Step 6.

### Synthesis of tert-butyl (6S)-2-(4-chloro-2-iodo-6-methylbenzyl)-6-methylmorpholine-4-carboxylate (27f)

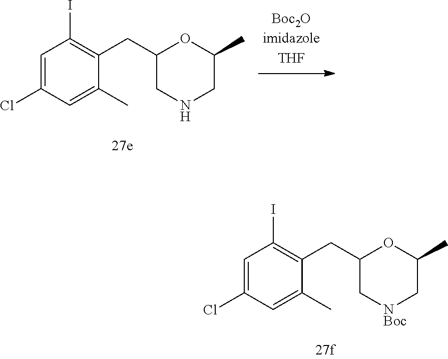 [SMILES: Cc1cc(Cl)cc(I)c1CC1CNC[C@H](C)O1 ; Cc1cc(Cl)cc(I)c1CC1CN(C(=O)OC(C)(C)C)C[C@H](C)O1]

To the solution of 27e (the crude product) in THF (3 mL) Boc₂O (116 mg; 0.53 mmol) and imidazole (73 mg; 1.08 mmol) were added and this mixture was then stirred at room temperature for 3 hours. The reaction progress was monitored by LC-MS. When analysis indicated completion of the reaction, the solvent was evaporated in vacuo and the residue was then purified twice by flash column chromatography on silica (first: hexane/AcOEt, 100:0 to 60:40, v/v, 8 min; second: hexane/AcOEt, 100:0 to 70:30, v/v, 15 min). Compound 27f was obtained in 39% yield (per two steps)(50 mg; 0.11 mmol).

ESI-MS m/z for C₁₈H₂₆ClINO₃ found 466.1/468.1 [M+H]⁺; Rₜ=1.95 min (Method A)

### Step 7.

### Synthesis of tert-butyl (6S)-2-(4-chloro-2-methyl-6-(4,4,5,5-tetramethyl-1,3,2-dioxaborolan-2-yl)benzyl)-6-methylmorpholine-4-carboxylate (27g)

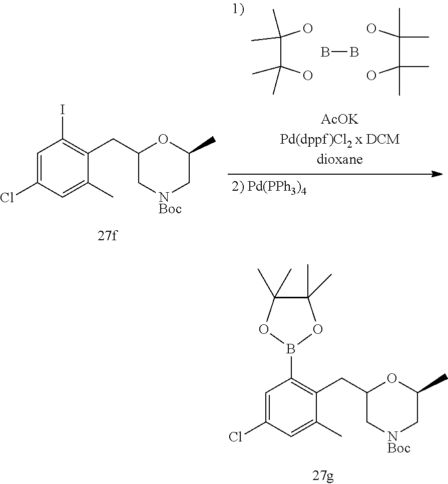

In a Schlenk flask was placed the compound 27f (47 mg; 0.100 mmol), bis(pinacolato)diboron (51 mg; 0.200 mmol), AcOK (30 mg; 0.300 mmol) and Pd(dppf)Cl₂×DCM (8 mg; 0.010 mmol) and dioxane (0.85 mL). The reaction mixture was intensively flushed with Ar. The mixture was capped and stirred at 90° C. overnight. Then Pd(PPh₃)₄ (6 mg; 0.005 mmol) was added and stirred at 90° C. overnight. Then another portion of bis(pinacolato)diboron (26 mg; 0.100 mmol) and Pd(dppf)Cl₂×DCM (8 mg; 0.010 mmol) was added and stirred at 90° C. overnight and after that another portion of Pd(PPh₃)₄ (11 mg; 0.009 mmol) was added and stirred at 90° C. overnight. The reaction progress was monitored by LC-MS. When analysis indicated completion of the reaction, the reaction mixture was used to the next step without any additional purification.

ESI-MS m/z for C₂₀H₃₀BClNO₅ found 409.8/411.8 [M+H-tBu]⁺; Rₜ=2.24 min (Method A)

### Step 8.

### Synthesis of tert-butyl (6S)-2-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylbenzyl)-6-methylmorpholine-4-carboxylate (27h)

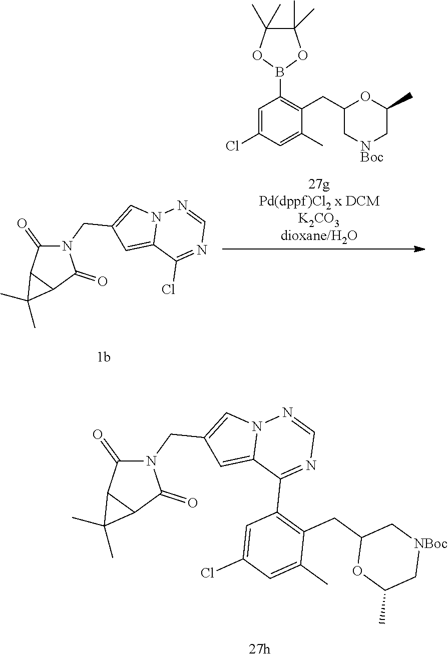 [SMILES: Cc1cc(Cl)cc(B2OC(C)(C)C(C)(C)O2)c1CC1CN(C(=O)OC(C)(C)C)C[C@H](C)O1 ; CC1(C)C2C(=O)N(Cc3cc4c(Cl)ncnn4c3)C(=O)C21 ; Cc1cc(Cl)cc(-c2ncnn3cc(CN4C(=O)C5C(C4=O)C5(C)C)cc23)c1CC1CN(C(=O)OC(C)(C)C)C[C@H](C)O1]

The title compound (27h) was obtained from 1b (30 mg; 0.100 mmol) and from 27g (the crude product) according to the General Procedure IV in 67% yield (per two steps)(37 mg; 0.061 mmol). The crude product was purified by flash column chromatography on silica (hexane/AcOEt, 100:0 to 50:50, v/v, 28 min).

ESI-MS m/z for C₃₂H₃₈ClN₅O₅ found 608.2/610.2 [M+H]⁺; Rₜ=1.97 min (Method A)

### Step 9.

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione dihydrochloride (27)

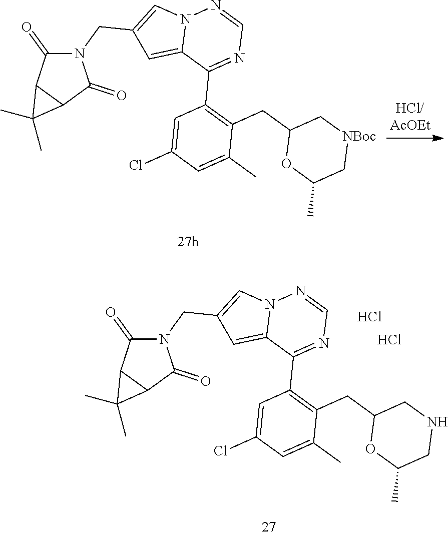 [SMILES: Cc1cc(Cl)cc(-c2ncnn3cc(CN4C(=O)C5C(C4=O)C5(C)C)cc23)c1CC1CN(C(=O)OC(C)(C)C)C[C@H](C)O1 ; Cc1cc(Cl)cc(-c2ncnn3cc(CN4C(=O)C5C(C4=O)C5(C)C)cc23)c1CC1CNC[C@H](C)O1 ; Cl ; Cl]

The title compound (27) was obtained as a dihydrochloride salt from 27h (37 mg; 0.061 mmol) according to the General Procedure IIIa in 5% yield (2 mg; 0.003 mmol). The crude product was purified by preparative reversed-phase column chromatography (C-18, water+0.3‰ HCl (36%)/MeCN, 99:1 to 30:70, 30 min, 20 mL/min, Rₜ=18.13 min).

ESI-MS m/z for C₂₇H₃₁ClN₅O₃ found 508.4/510.4 [M+H]⁺; Rₜ=1.26 min (Method A); ¹H NMR (700 MHz, D₂O) δ 8.44 (s, 1H), 8.09-8.05 (m, 1H), 7.46-7.43 (m, 1H), 7.22-7.19 (m, 1H), 6.73-6.70 (m, 1H), 4.59 (s, 2H), 3.69-3.64 (m, 1H), 3.46-3.40 (m, 1H), 3.16-3.12 (m, 1H), 3.06-3.00 (m, 2H), 2.93-2.87 (m, 1H), 2.55-2.47 (m, 3H), 2.42-2.37 (m, 1H), 2.34 (s, 3H), 1.14 (s, 3H), 0.96 (s, 3H), 0.60 (d, J=6.2 Hz, 3H).

### Example 28

### Synthesis of 3-((4-(5-chloro-2-(((S)-5,5-difluoropiperidin-3-yl)oxy)-3-methylphenyl)pyrrolo[2,1-f][1,2,4]triazin-6-yl)methyl)-6,6-dimethyl-3-azabicyclo[3.1.0]hexane-2,4-dione 2,2,2-trifluoroacetate (28)

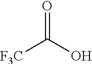

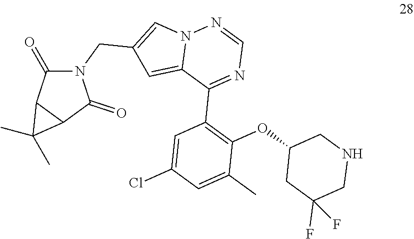 [SMILES: Cc1cc(Cl)cc(-c2ncnn3cc(CN4C(=O)C5C(C4=O)C5(C)C)cc23)c1O[C@@H]1CNCC(F)(F)C1]

### Step 1.

### Synthesis of tert-butyl (3S,5R)-3-(2-bromo-4-chloro-6-methylphenoxy)-5-((tert-butyldimethylsilyl)oxy)piperidine-1-carboxylate (28a)

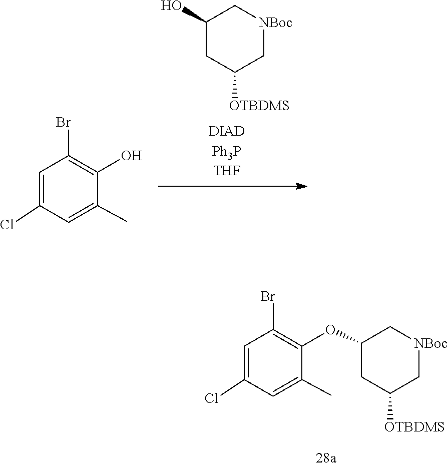

To a cooled to −10° C. solution of 2-bromo-4-chloro-6-methylphenol (134 mg; 0.604 mmol), tert-butyl (3R,5R)-3-((tert-butyldimethylsilyl)oxy)-5-hydroxypiperidine-1-carboxylate (200 mg; 0.604 mmol) and PPh₃ (190 mg; 0.725 mmol) in THE (3 ml) DIAD (0.155 mL; 0.785 mmol) was slowly added. The resulting mixture was stirred at room temperature overnight. Then the reaction mixture was concentrated in vacuo and the residue was purified by flash column chromatography on silica (hexane/AcOEt, 100:0 to 70:30, v/v, 25 min, 20 mL/min). Compound 28a was obtained in 83% yield (267 mg; 0.501 mmol).

ESI-MS m/z for C₁₉H₃₀BrClNO₄Si found 478.0/479.9 [M+H-tBu]⁺; Rₜ=2.78 min (Method A)

### Step 2.

### Synthesis of tert-butyl (3S,5R)-3-(2-bromo-4-chloro-6-methylphenoxy)-5-hydroxypiperidine-1-carboxylate (28b)

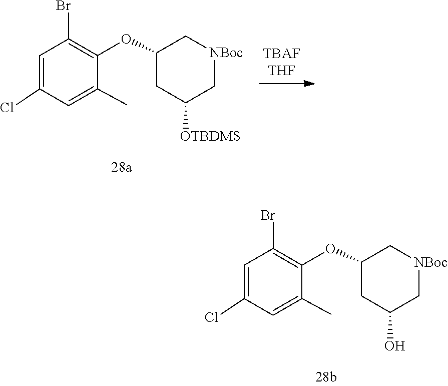 [SMILES: Cc1cc(Cl)cc(Br)c1O[C@H]1C[C@@H](O[Si](C)(C)C(C)(C)C)CN(C(=O)OC(C)(C)C)C1 ; Cc1cc(Cl)cc(Br)c1O[C@H]1C[C@@H](O)CN(C(=O)OC(C)(C)C)C1]

To a solution of 28a (265 mg; 0.50 mmol) in THF (2 ml) TBAF (1 M in THF; 0.74 mL; 0.74 mmol) was added. The resulting mixture was stirred at room temperature for 2 hours. Then the reaction mixture was concentrated in vacuo and the residue was purified by flash column chromatography on silica (hexane/AcOEt, 100:0 to 20:80, v/v, 27 min, 15 mL/min). Compound 28b was obtained in 70% yield (145 mg; 0.35 mmol).

ESI-MS m/z for C₁₃H₁₆BrClNO₄ found 363.8/365.8 [M+H-tBu]⁺; Rₜ=1.71 min (Method A); ¹H NMR (700 MHz, CDCl₃) δ 7.42 (s, 1H), 7.14 (s, 1H), 4.27-4.16 (m, 1H), 3.99-3.72 (m, 3H), 3.66-3.53 (m, 1H), 3.26-3.12 (m, 1H), 2.38-2.34 (m, 1H), 2.32 (s, 3H), 2.03-1.92 (m, 1H), 1.47 (s, 9H).

### Step 3.

### Synthesis of tert-butyl (S)-3-(2-bromo-4-chloro-6-methylphenoxy)-5-oxopiperidine-1-carboxylate (28c)

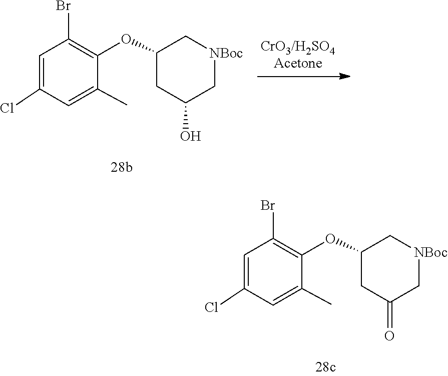 [SMILES: Cc1cc(Cl)cc(Br)c1O[C@H]1C[C@@H](O)CN(C(=O)OC(C)(C)C)C1 ; Cc1cc(Cl)cc(Br)c1O[C@H]1CC(=O)CN(C(=O)OC(C)(C)C)C1]

To a solution of 28b (110 mg; 0.261 mmol) in acetone (5 mL) at 0° C., Jones reagent (2.6 M; 0.25 mL; 0.654 mmol) was added dropwise. The reaction mixture was stirred at 0° C. for 1 hour, and then isopropanol (iPrOH) (0.5 mL) and ethyl acetate (20 mL) were added and the mixture was stirred for 15 minutes and then filtered through a pad of the Celite. The filtrate was washed with brine (2×20 mL), dried over anhydrous MgSO₄ and concentrated in vacuo and the residue was purified by flash column chromatography on silica (hexane/AcOEt, 100:0 to 20:80, v/v, 27 min, 10 mL/min). Compound 28c was obtained in 28% yield (30 mg; 0.072 mmol).

### Step 4.

### Synthesis of tert-butyl (S)-5-(2-bromo-4-chloro-6-methylphenoxy)-3,3-difluoropiperidine-1-carboxylate (28d)

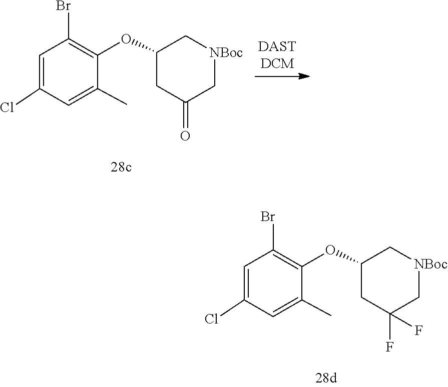 [SMILES: Cc1cc(Cl)cc(Br)c1O[C@H]1CC(=O)CN(C(=O)OC(C)(C)C)C1 ; Cc1cc(Cl)cc(Br)c1O[C@@H]1CN(C(=O)OC(C)(C)C)CC(F)(F)C1]

To a cooled to −20° C. solution of 28c (30 mg; 0.072 mmol) in dry DCM (2 mL) DAST (24 μL; 0.180 mmol) was added dropwise. Then, this mixture was stirred at 0° C. for 15 minutes and then at room temperature for 1 hour. Next, the reaction was quenched with 5% NaHCO₃(3 mL) and then diluted with DCM (15 mL). The phases were separated and an aqueous one was extracted with DCM (2×10 mL). The combined organic solutions were washed with water (10 mL) and brine (10 mL) and then dried over anhydrous Na₂SO₄, filtered and concentrated in vacuo. The residue was purified by flash column chromatography on silica (hexane/AcOEt, 100:0 to 20:80, v/v, 27 min, 10 mL/min). Compound 28d was obtained in 79% yield (25 mg; 0.057 mmol).

### Step 5.

### Synthesis of tert-butyl (S)-5-(4-chloro-2-methyl-6-(4,4,5,5-tetramethyl-1,3,2-dioxaborolan-2-yl)phenoxy)-3,3-difluoropiperidine-1-carboxylate (28e)

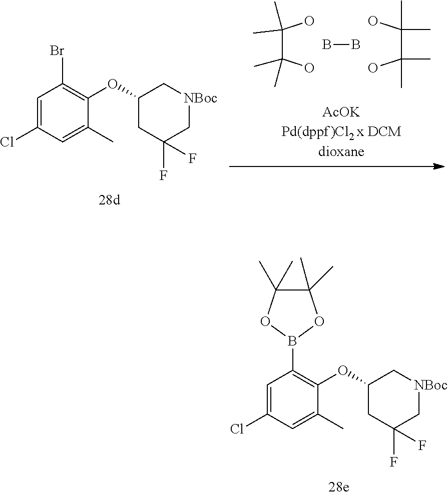

In a Schlenk flask was placed the compound 28d (22 mg; 0.050 mmol), bis(pinacolato)diboron (25 mg; 0.100 mmol), AcOK (15 mg; 0.150 mmol), Pd(dppf)Cl₂×DCM (4.1 mg; 0.005 mmol) and dioxane (0.7 mL). The reaction mixture was intensively flushed with Ar. The mixture was capped and stirred at 90° C. overnight. Then, the reaction mixture was concentrated in vacuo and the crude product was used to the next step without any additional purification.

### Step 6.

### Synthesis of tert-butyl (5S)-5-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenoxy)-3,3-difluoropiperidine-1-carboxylate (28f)

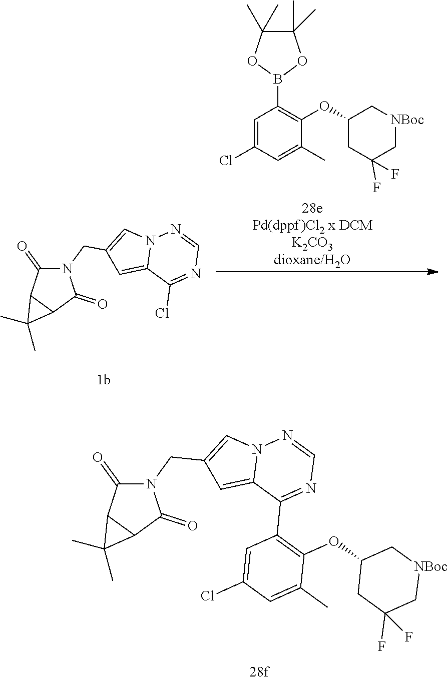 [SMILES: Cc1cc(Cl)cc(B2OC(C)(C)C(C)(C)O2)c1O[C@@H]1CN(C(=O)OC(C)(C)C)CC(F)(F)C1 ; CC1(C)C2C(=O)N(Cc3cc4c(Cl)ncnn4c3)C(=O)C21 ; Cc1cc(Cl)cc(-c2ncnn3cc(CN4C(=O)C5C(C4=O)C5(C)C)cc23)c1O[C@@H]1CN(C(=O)OC(C)(C)C)CC(F)(F)C1]

The title compound (28f) was obtained from 1b (15 mg; 0.050 mmol) and from 28e (the crude product) according to the General Procedure IV. The crude product was purified by flash column chromatography on silica (hexane/AcOEt, 100:0 to 0:100, v/v, 21 min, 15 mL/min) and then was used to the next step without any additional purification.

### Step 7.

### Synthesis of 3-((4-(5-chloro-2-(((S)-5,5-difluoropiperidin-3-yl)oxy)-3-methylphenyl)pyrrolo[2,1-f][1,2,4]triazin-6-yl)methyl)-6,6-dimethyl-3-azabicyclo[3.1.0]hexane-2,4-dione 2,2,2-trifluoroacetate (28)

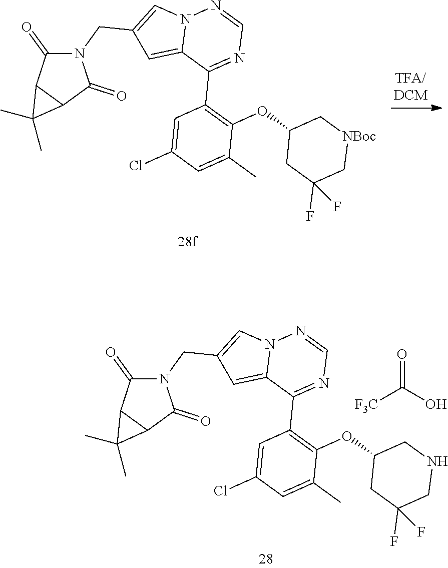

The title compound (2) was obtained as a TA salt from 28f the crude product) according to the General Procedure IIIb in 2% yield (per two steps)(1 mg; 0.001 mmol). The crude product was purified by preparative reversed-phase column chromatography (column: Cosmosil Cholester 20×250 mm, water/MeCN+1‰ TFA, 99:1 to 20:80, 30 min, 18 mL/min, Rₜ=18.8 min).

ESI-MS m/z for C₂₈H₂₉ClFN₆O₂ found 530.3/532.3 [M+H]⁺; Rₜ=1.28 min (Method A); ¹H NMR (700 MHz, D₂O) δ 8.43 (s, 1H), 8.02-7.99 (m, 1H), 7.51-7.47 (m, 1H), 7.35-7.31 (m, 1H), 6.76-6.72 (m, 1H), 4.64-4.63 (m, 2H), 4.07-4.00 (m, 1H), 3.41-3.35 (m, 1H), 3.32-3.26 (m, 1H), 3.18-3.05 (m, 2H), 2.52 (s, 2H), 2.30 (s, 3H), 2.11-2.03 (m, 1H), 1.96-1.86 (m, 1H), 1.14 (s, 3H), 0.95 (s, 3H).

### Example 29

### Synthesis of 3-((4-(5-chloro-1-((3-fluoropiperidin-3-yl)methyl)-1H-indol-7-yl)pyrrolo[2,1-f][1,2,4]triazin-6-yl)methyl)-6,6-dimethyl-3-azabicyclo[3.1.0]hexane-2,4-dione hydrochloride (29)

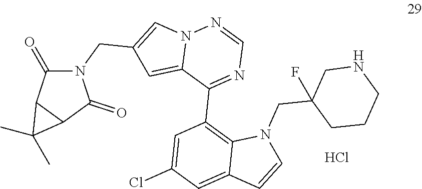 [SMILES: CC1(C)C2C(=O)N(Cc3cc4c(-c5cc(Cl)cc6ccn(CC7(F)CCCNC7)c56)ncnn4c3)C(=O)C21 ; Cl]

The title compound (29) was obtained as a hydrochloride salt in 1% overall yield in a similar way to Example 5 with the exception that, in the first step of the synthesis, 7-bromo-5-chloro-1H-indole and tert-butyl 3-fluoro-3-(((methylsulfonyl)oxy)methyl)piperidine-1-carboxylate 29a (the synthesis of this compound was described below) were used instead of 7-bromo-5-(trifluoromethyl)-1H-indole and tert-butyl 3-((methylsulfonyl)oxy)pyrrolidine-1-carboxylate and to this reaction 0.36 equivalents of KI was added. The crude product was purified by preparative reversed-phase column chromatography (C-18, water+0.3‰ HCl (36%)/MeCN, 95:5 to 35:65, 30 min, 20 mL/min, Rₜ=18.7 min).

ESI-MS m/z for C₂₈H₂₉ClFN₆O₂ found 535.3/537.3 [M+H]⁺; Rₜ=1.29 min (Method A); ¹H NMR (700 MHz, D₂O) δ 8.48 (s, 1H), 8.05-8.01 (m, 1H), 7.83-7.80 (m, 1H), 7.30-7.25 (m, 2H), 6.71-6.69 (m, 1H), 6.63 (s, 1H), 4.60-4.57 (m, 2H), 4.21-4.06 (m, 2H), 3.19-3.12 (m, 1H), 2.88-2.75 (m, 1H), 2.72-2.63 (m, 2H), 2.48 (s, 2H), 1.59-1.54 (m, 1H), 1.51-1.43 (m, 1H), 1.34-1.18 (m, 2H), 1.13 (s, 3H), 0.93 (s, 3H).

### Synthesis of tert-butyl 3-fluoro-3-(((methylsulfonyl)oxy)methyl)piperidine-1-carboxylate (29a)

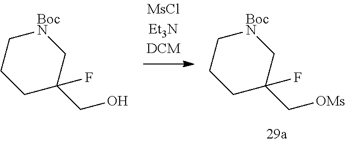 [SMILES: CC(C)(C)OC(=O)N1CCCC(F)(CO)C1 ; CC(C)(C)OC(=O)N1CCCC(F)(COS(C)(=O)=O)C1]

The title compound (29a) was obtained from tert-butyl 3-fluoro-3-(hydroxymethyl)piperidine-1-carboxylate (753 mg; 3.23 mmol) according to the General Procedure V and after standard work-up the crude product 29a (99% yield; 998 mg; 3.21 mmol) was taken to the next step.

ESI-MS m/z for C₈H₁₅FNO₅S found 256.0 [M+H-tBu]⁺; Rₜ=1.27 min (Method A); ¹H NMR (700 MHz, CDCl₃) δ 4.33-4.19 (m, 2H), 3.87-3.78 (m, 1H), 3.71-3.59 (m, 1H), 3.37-3.26 (m, 1H), 3.22-3.14 (m, 1H), 3.08 (s, 3H), 1.95-1.89 (m, 1H), 1.84-1.72 (m, 2H), 1.57-1.54 (m, 1H), 1.46 (s, 9H).

### Example 30

### Synthesis of 3-((4-(5-chloro-1-(((S)-morpholin-2-yl)methyl)-1H-indol-7-yl)pyrrolo[2,1-f][1,2,4]triazin-6-yl)methyl)-6,6-dimethyl-3-azabicyclo[3.1.0]hexane-2,4-dione dihydrochloride (30)

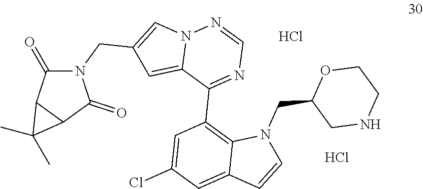 [SMILES: CC1(C)C2C(=O)N(Cc3cc4c(-c5cc(Cl)cc6ccn(C[C@@H]7CNCCO7)c56)ncnn4c3)C(=O)C21 ; Cl ; Cl]

### Step 1.

### Synthesis of 6-(((tert-butyldimethylsilyl)oxy)methyl)-4-chloropyrrolo[2,1-f][1,2,4]triazine (30a)

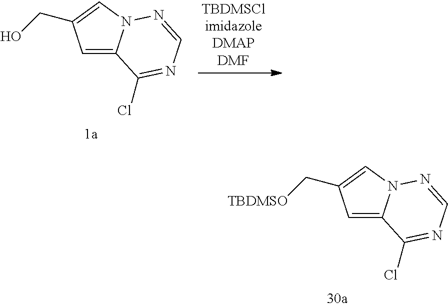 [SMILES: OCc1cc2c(Cl)ncnn2c1 ; CC(C)(C)[Si](C)(C)OCc1cc2c(Cl)ncnn2c1]

To the solution of 1a (880 mg; 4.793 mmol) in DMF (12 mL) TBDMSCl (867 mg; 5.752 mmol), imidazole (653 mg; 9.586 mmol) and DMAP (59 mg; 0.479 mmol) were added and this mixture was then stirred at room temperature for 1.5 hours. The reaction progress was monitored by LC-MS. When analysis indicated completion of the reaction, the solvent was evaporated in vacuo and the residue was dissolved in Et₂O and water. The phases were separated and an aqueous one was extracted with Et₂O (2×) and AcOEt (1×). The organic layer was washed with 5% NaHCO₃ and brine. The combined organic solutions were then dried over anhydrous Na₂SO₄, filtered and concentrated in vacuo. The residue was purified by flash column chromatography on silica (hexane/AcOEt, 100:0 to 40:60, v/v, 40 min, 35 mL/min). Compound 30a was obtained in 77% yield (1.091 g; 3.672 mmol).

ESI-MS m/z for C₁₃H₂₁ClN₃OSi found 298.0/300.0 [M+H]⁺; Rₜ=2.03 min (Method A); ¹H NMR (700 MHz, CDCl₃) δ 8.18 (s, 1H), 7.85-7.83 (m, 1H), 6.90-6.87 (m, 1H), 4.91-4.85 (m, 2H), 0.96 (s, 9H), 0.13 (s, 6H).

### Step 2.

### Synthesis of tert-butyl (R)-2-((7-bromo-5-chloro-1H-indol-1-yl)methyl)morpholine-4-carboxylate (30b)

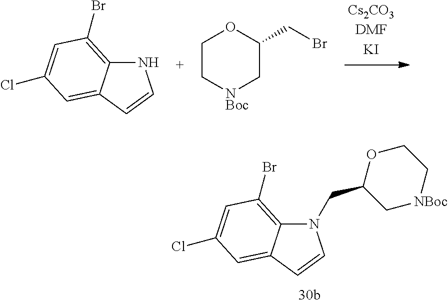 [SMILES: Clc1cc(Br)c2[nH]ccc2c1 ; CC(C)(C)OC(=O)N1CCO[C@H](CBr)C1 ; CC(C)(C)OC(=O)N1CCO[C@H](Cn2ccc3cc(Cl)cc(Br)c32)C1]

The solution of 7-bromo-5-chloro-1H-indole (433 mg; 1.879 mmol), tert-butyl (S)-2-(bromomethyl)morpholine-4-carboxylate (679 mg; 2.067 mmol), Cs₂CO₃ (1.224 g; 3.758 mmol) and KI (112 mg; 0.676 mmol) in dry DMF (5 mL) was stirred under argon atmosphere at 80° C. overnight. Then another portion of Cs₂CO₃ (306 mg; 0.939 mmol) and KI (31 mg; 0.189 mmol) were added and a whole was stirred at 90° C. for 5 hours. The reaction progress was monitored by LC-MS. When analysis indicated completion of the reaction, a whole was concentrated in vacuo. The residue was partitioned between AcOEt and water. The phases were separated and an aqueous one was extracted with AcOEt. The combined organic solutions were washed with brine, dried over anhydrous Na₂SO₄, filtered and concentrated in vacuo. The crude product was then purified by flash column chromatography on silica (hexane/AcOEt, 100:0 to 40:60, v/v, 35 min). Compound 30b was obtained in 68% yield (549 mg; 1.282 mmol).

ESI-MS m/z for C₁₄H₁₅BrClN₂O₃ found 373.0/375.0 [M+H-tBu]⁺; Rₜ=1.97 min (Method A); ¹H NMR (700 MHz, CDCl₃) δ 7.54-7.50 (m, 1H), 7.38-7.34 (m, 1H), 7.15-7.12 (m, 1H), 6.43-6.39 (m, 1H), 4.80-4.73 (m, 1H), 4.44-4.35 (m, 1H), 4.07-3.77 (m, 4H), 3.46-3.40 (m, 1H), 2.97-2.88 (m, 1H), 2.66-2.56 (m, 1H), 1.44 (s, 9H).

### Step 3.

### Synthesis of tert-butyl (R)-2-((5-chloro-7-(4,4,5,5-tetramethyl-1,3,2-dioxaborolan-2-yl)-1H-indol-1-yl)methyl)morpholine-4-carboxylate (30c)

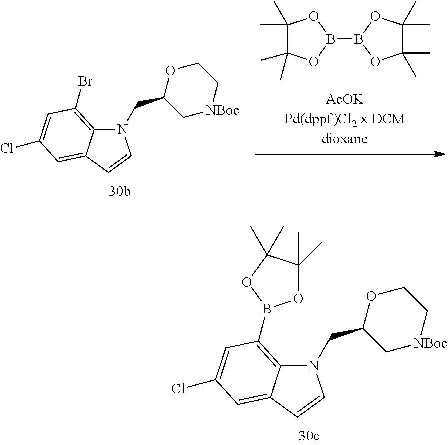

In a Schlenk flask was placed the compound 30b (548 mg; 1.275 mmol), bis(pinacolato)diboron (648 mg; 2.550 mmol), AcOK (375 mg; 3.825 mmol), Pd(dppf)Cl₂×DCM (104 mg; 0.127 mmol) and dioxane (5 mL). The reaction mixture was intensively flushed with Ar. The mixture was capped and stirred at 90° C. overnight. The reaction progress was monitored by LC-MS. When analysis indicated completion of the reaction, the reaction mixture was concentrated in vacuo. The residue was then used to the next step without any additional purification.

ESI-MS m/z for C₂₀H₂₇BClN₂O₅ found 421.0/423.0 [M+H-tBu]⁺; Rₜ=2.16 min (Method A)

### Step 4.

### Synthesis of tert-butyl (R)-2-((7-(6-(((tert-butyldimethylsilyl)oxy)methyl)pyrrolo[2,1-f][1,2,4]triazin-4-yl)-5-chloro-1H-indol-1-yl)methyl)morpholine-4-carboxylate (30d)

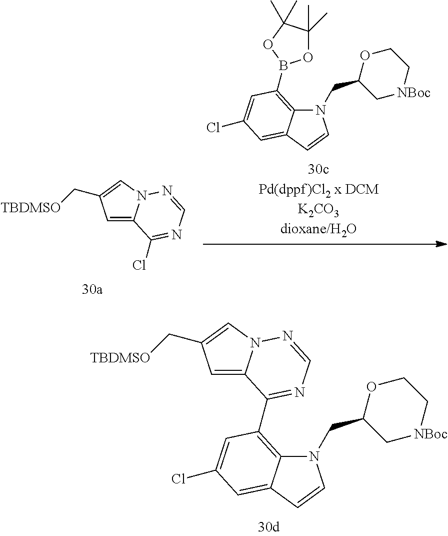 [SMILES: CC(C)(C)OC(=O)N1CCO[C@H](Cn2ccc3cc(Cl)cc(B4OC(C)(C)C(C)(C)O4)c32)C1 ; CC(C)(C)[Si](C)(C)OCc1cc2c(Cl)ncnn2c1 ; CC(C)(C)OC(=O)N1CCO[C@H](Cn2ccc3cc(Cl)cc(-c4ncnn5cc(CO[Si](C)(C)C(C)(C)C)cc45)c32)C1]

The title compound (30d) was obtained from 30a (760 mg; 2.550 mmol) and from 30c (the crude product) according to the General Procedure IV. The crude product was purified by flash column chromatography on silica (hexane/AcOEt, 100:0 to 70:30, v/v, 39 min). Compound 30d was obtained in 42% yield (per two steps)(327 mg; 0.535 mmol).

ESI-MS m/z for C₃₁H₄₃ClN₅O₄Si found 612.0/614.0 [M+H]⁻; Rₜ=2.48 min (Method A)

### Step 5.

### Synthesis of tert-butyl (R)-2-((5-chloro-7-(6-(hydroxymethyl)pyrrolo[2,1-f][1,2,4]triazin-4-yl)-1H-indol-1-yl)methyl)morpholine-4-carboxylate (30e)

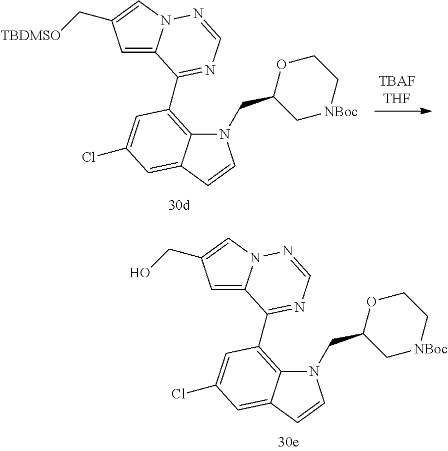 [SMILES: CC(C)(C)OC(=O)N1CCO[C@H](Cn2ccc3cc(Cl)cc(-c4ncnn5cc(CO[Si](C)(C)C(C)(C)C)cc45)c32)C1 ; CC(C)(C)OC(=O)N1CCO[C@H](Cn2ccc3cc(Cl)cc(-c4ncnn5cc(CO)cc45)c32)C1]

To a solution of 30d (200 mg; 0.327 mmol) in THF (2 ml) TBAF (1 M in THF; 0.50 mL; 0.500 mmol) was added. The resulting mixture was stirred at room temperature for 1 hour. Then the reaction mixture was concentrated in vacuo and the residue was purified by flash column chromatography on silica (hexane/AcOEt, 100:0 to 0:100, v/v, 22 min, 13 mL/min). Compound 30e was obtained in 74% yield (120 mg; 0.241 mmol).

ESI-MS m/z for C₂₅H₂₉ClN₅O₄ found 498.1/500.0 [M+H]⁺; Rₜ=1.55 min (Method A); ¹H NMR (700 MHz, CDCl₃) δ 8.55 (s, 1H), 7.98 (s, 1H), 7.78-7.70 (m, 1H), 7.44-7.34 (m, 1H), 7.20 (s, 1H), 6.80-6.75 (m, 1H), 6.59-6.51 (m, 1H), 4.82 (s, 2H), 4.05-3.83 (m, 2H), 3.71-3.49 (m, 2H), 3.31-3.20 (m, 2H), 3.13-2.78 (m, 2H), 2.66-2.59 (m, 1H), 1.39 (s, 9H).

### Step 6.

### Synthesis of tert-butyl (2R)-2-((5-chloro-7-(6-((6,6-dimethyl-2,4-dioxo-3-azabicyclo[3.1.0]hexan-3-yl)methyl)pyrrolo[2,1-f][1,2,4]triazin-4-yl)-1H-indol-1-yl)methyl)morpholine-4-carboxylate (30f)

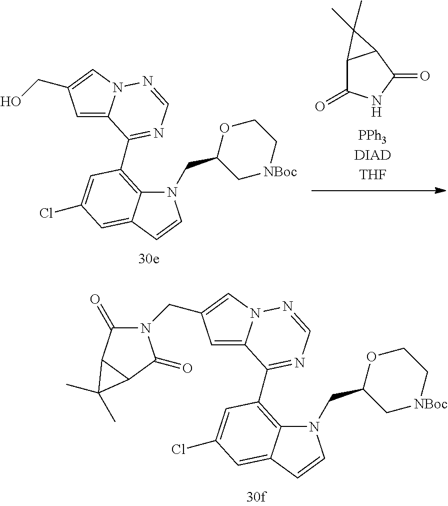

To a cooled to −10° C. solution of 30e (10 mg; 0.020 mmol), succinimide (2.8 mg; 0.020 mmol) and PPh₃ (6.3 mg; 0.024 mmol) in THE (0.5 mL) DIAD (5 μL; 0.026 mmol) was slowly added. The resulting mixture was stirred at room temperature overnight. The reaction progress was monitored by LC-MS. When analysis indicated almost completion of the reaction, the solvent was evaporated in vacuo and the residue was purified by flash column chromatography on silica (hexane/AcOEt, 100:0 to 0:100, v/v, 20 min). Compound 30f was obtained in 70% yield (9 mg; 0.014 mmol).

ESI-MS m/z for C₃₂H₃₆ClN₆O₅ found 619.2/621.3 [M+H]⁺; Rₜ=1.82 min (Method A)

### Step 7.

### Synthesis of 3-((4-(5-chloro-1-(((S)-morpholin-2-yl)methyl)-1H-indol-7-yl)pyrrolo[2,1-f][1,2,4]triazin-6-yl)methyl)-6,6-dimethyl-3-azabicyclo[3.1.0]hexane-2,4-dione dihydrochloride (30)

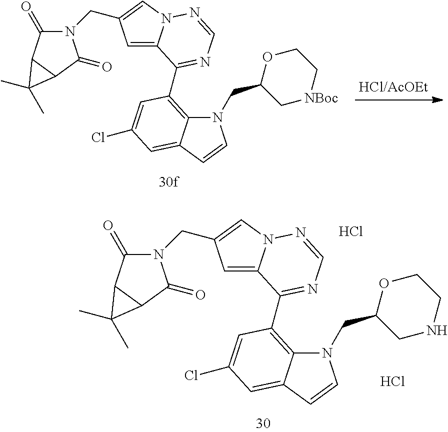 [SMILES: CC(C)(C)OC(=O)N1CCO[C@H](Cn2ccc3cc(Cl)cc(-c4ncnn5cc(CN6C(=O)C7C(C6=O)C7(C)C)cc45)c32)C1 ; CC1(C)C2C(=O)N(Cc3cc4c(-c5cc(Cl)cc6ccn(C[C@@H]7CNCCO7)c56)ncnn4c3)C(=O)C21 ; Cl ; Cl]

The title compound (30) was obtained as a dihydrochloride salt as a single isomer (enantiomer) from 30f (9 mg; 0.014 mmol) according to the General Procedure IIIa in 71% yield (6 mg; 0.010 mmol). The crude product was purified by preparative reversed-phase column chromatography (column: Cosmosil Cholester 20×250 mm, water+0.3‰ HCl (36%)/MeCN, 95:5 to 20:80, 30 min, 20 mL/min, Rₜ=17.0 min).

ESI-MS m/z for C₂₇H₂₈ClN₆O₃ found 519.3/521.3 [M+H]⁺; Rₜ=1.33 min (Method A); ¹H NMR (700 MHz, Methanol-d₄) δ 8.59 (s, 1H), 8.12-8.09 (m, 1H), 7.86-7.78 (m, 1H), 7.41-7.38 (m, 1H), 7.37-7.33 (m, 1H), 6.81-6.75 (m, 1H), 6.69-6.67 (m, 1H), 4.68 (s, 2H), 4.23-4.07 (m, 2H), 3.66-3.59 (m, 1H), 3.47-3.41 (m, 1H), 3.26-3.17 (m, 1H), 3.05-2.91 (m, 2H), 2.73-2.66 (m, 1H), 2.50-2.39 (m, 3H), 1.24 (s, 3H), 1.10 (s, 3H).

### Example 31

### Synthesis of (S)-2-((4-(5-chloro-1-(((S)-morpholin-2-yl)methyl)-1H-indol-7-yl)pyrrolo[2,1-f][1,2,4]triazin-6-yl)methyl)tetrahydropyrrolo[1,2-a]pyrazine-1,3(2H,4H)-dione dihydrochloride (31)

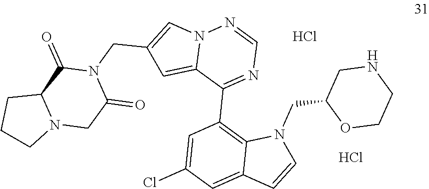 [SMILES: O=C1CN2CCC[C@H]2C(=O)N1Cc1cc2c(-c3cc(Cl)cc4ccn(C[C@@H]5CNCCO5)c34)ncnn2c1 ; Cl ; Cl]

### Step 1.

### Synthesis of ethyl (S)-2-(2-carbamoylpyrrolidin-1-yl)acetate (31a)

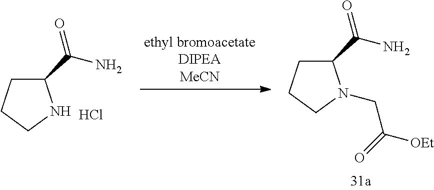 [SMILES: NC(=O)[C@@H]1CCCN1 ; Cl ; CCOC(=O)CN1CCC[C@H]1C(N)=O]

To a cooled to 0° C. solution of (S)-pyrrolidine-2-carboxamide hydrochloride (150 mg; 1.00 mmol) and DIPEA (0.43 mL; 2.50 mmol) in MeCN (10 mL) ethyl bromoacetate (111 μL; 1.00 mmol) was added dropwise. The resulting mixture was stirred at room temperature overnight. The reaction progress was monitored by LC-MS. When analysis indicated completion of the reaction, the solvent was evaporated in vacuo and the residue was purified by flash column chromatography on silica (hexane/AcOEt, 100:00 to 0:100, v/v, 30 min, 15 mL/min). Compound 31a was obtained as a white solid in 77% yield (154 mg; 0.77 mmol).

ESI-MS m/z for C₉H₁₇N₂O₃ found 201.0 [M+H]⁺; Rₜ=0.16 min (Method A); ¹H NMR (700 MHz, CDCl₃) δ 4.19 (qd, J=7.1, 0.7 Hz, 2H), 3.53 (d, J=17.1 Hz, 1H), 3.39 (d, J=17.1 Hz, 1H), 3.36-3.31 (m, 1H), 3.29-3.22 (m, 1H), 2.68-2.58 (m, 1H), 2.28-2.19 (m, 1H), 2.04-1.97 (m, 1H), 1.88-1.79 (m, 2H), 1.28 (t, J=7.1 Hz, 3H).

### Step 2.

### Synthesis of (S)-tetrahydropyrrolo[1,2-a]pyrazine-1,3(2H,4H)-dione (31b)

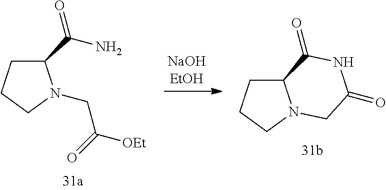 [SMILES: CCOC(=O)CN1CCC[C@H]1C(N)=O ; O=C1CN2CCC[C@H]2C(=O)N1]

To a solution of 31a (150 mg; 0.75 mmol) in absolute EtOH (5.5 mL) NaOH (30 mg; 0.75 mmol) was added. The resulting mixture was stirred at room temperature for 1 hour. The reaction progress was monitored by LC-MS. When analysis indicated completion of the reaction, the reaction mixture was quenched with saturated solution of NH₄Cl (50 mL) and extracted with DCM (3×30 mL). The organic layer was dried over anhydrous Na₂SO₄, filtered and concentrated in vacuo. Compound 31b was obtained as a white solid in 89% yield (104 mg; 0.67 mmol).

ESI-MS m/z for C₇H₁₁N₂O₂ found 155.1 [M+H]⁺; Rₜ=0.16 min (Method A); ¹H NMR (700 MHz, CDCl₃) δ 3.79 (d, J=17.3 Hz, 1H), 3.52 (d, J=17.3 Hz, 1H), 3.41-3.33 (m, 1H), 2.90-2.84 (m, 1H), 2.77-2.68 (m, 1H), 2.27-2.17 (m, 2H), 1.94-1.85 (m, 2H).

### Step 3.

### Synthesis of tert-butyl (R)-2-((7-(6-(bromomethyl)pyrrolo[2,1-f][1,2,4]triazin-4-yl)-5-chloro-1H-indol-1-yl)methyl)morpholine-4-carboxylate (31c)

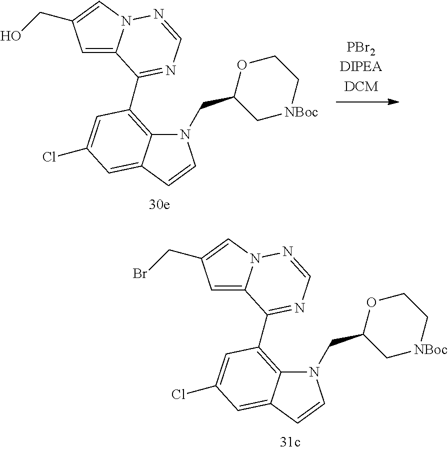 [SMILES: CC(C)(C)OC(=O)N1CCO[C@H](Cn2ccc3cc(Cl)cc(-c4ncnn5cc(CO)cc45)c32)C1 ; CC(C)(C)OC(=O)N1CCO[C@H](Cn2ccc3cc(Cl)cc(-c4ncnn5cc(CBr)cc45)c32)C1]

To a cooled to 0° C. solution of 30e (40 mg; 0.080 mmol) and DIPEA (52 μL; 0.320 mmol) in dry DCM (2 mL) PBr₃ (7.5 μL; 0.080 mmol) was slowly added. The resulting mixture was stirred at 0° C. for 90 minutes. The reaction progress was monitored by LC-MS. When analysis indicated completion of the reaction, the reaction mixture was quenched with a saturated solution of NaHCO₃(10 mL) and extracted with DCM (1×15 mL). The organic layer was washed with 1 M KHSO₄ (1×10 mL) and brine (1×10 mL), dried over anhydrous Na₂SO₄, filtered and concentrated in vacuo. Compound 31c was obtained in 99% yield (44 mg; 0.079 mmol).

ESI-MS m/z for C₂₅H₂₈BrClN₅O₃ found 560.1/562.0 [M+H]⁺; Rₜ=1.96 min (Method A)

### Step 4.

### Synthesis of tert-butyl (R)-2-((5-chloro-7-(6-(((S)-1,3-dioxohexahydropyrrolo[1,2-a]pyrazin-2(1H)-yl)methyl)pyrrolo[2,1-f][1,2,4]triazin-4-yl)-1H-indol-1-yl)methyl)morpholine-4-carboxylate (31d)

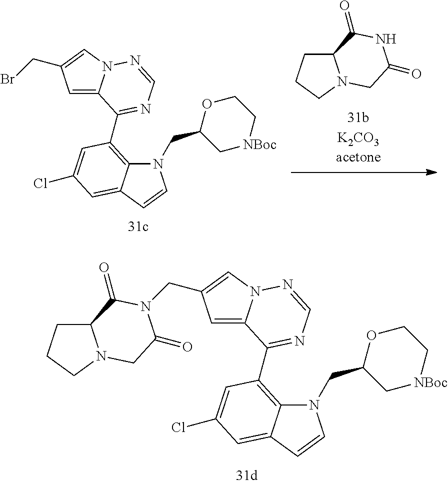 [SMILES: CC(C)(C)OC(=O)N1CCO[C@H](Cn2ccc3cc(Cl)cc(-c4ncnn5cc(CBr)cc45)c32)C1 ; O=C1CN2CCC[C@H]2C(=O)N1 ; CC(C)(C)OC(=O)N1CCO[C@H](Cn2ccc3cc(Cl)cc(-c4ncnn5cc(CN6C(=O)CN7CCC[C@H]7C6=O)cc45)c32)C1]

To a solution of 31c (44 mg; 0.079 mmol) in acetone (1 mL) K₂CO₃ (17 mg; 0.120 mmol) and the compound 31b (12 mg; 0.080 mmol) were added. The resulting mixture was capped and stirred at 80° C. for 1 hour. The reaction progress was monitored by LC-MS. When analysis indicated almost completion of the reaction, a whole was concentrated in vacuo and the residue was purified by flash column chromatography on silica (hexane/AcOEt, 100:0 to 0:100, v/v, 27 min, 15 mL/min). Compound 31d was obtained in 30% yield (15 mg; 0.024 mmol).

ESI-MS m/z for C₃₂H₃₇ClN₇O₅ found 634.2/636.1 [M+H]⁺; Rₜ=1.79 min (Method A)

### Step 5.

### Synthesis of (S)-2-((4-(5-chloro-1-(((S)-morpholin-2-yl)methyl)-1H-indol-7-yl)pyrrolo[2,1-f][1,2,4]triazin-6-yl)methyl)tetrahydropyrrolo[1,2-a]pyrazine-1,3(2H,4H)-dione dihydrochloride (31)

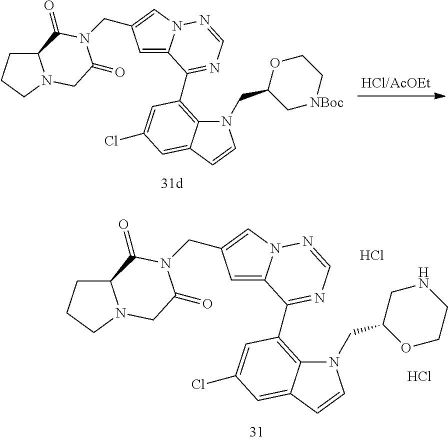 [SMILES: CC(C)(C)OC(=O)N1CCO[C@H](Cn2ccc3cc(Cl)cc(-c4ncnn5cc(CN6C(=O)CN7CCC[C@H]7C6=O)cc45)c32)C1 ; O=C1CN2CCC[C@H]2C(=O)N1Cc1cc2c(-c3cc(Cl)cc4ccn(C[C@@H]5CNCCO5)c34)ncnn2c1 ; Cl ; Cl]

The title compound (31) was obtained as a dihydrochloride salt as a single isomer from 31d (15 mg; 0.024 mmol) according to the General Procedure IIIa in 21% yield (3 mg; 0.005 mmol). The crude product was purified by preparative reversed-phase column chromatography (C-18, water+0.3‰ HCl (36%)/MeCN, 99:1 to 20:80, 30 min, 20 mL/min, Rₜ=15.75 min).

ESI-MS m/z for C₂₇H₂₉ClN₇O₃ found 534.4/536.1 [M+H]⁺; Rₜ=1.26 min (Method A); ¹H NMR (700 MHz, Methanol-d₄) δ 8.58 (s, 1H), 8.14 (d, J=1.5 Hz, 1H), 7.83 (d, J=2.1 Hz, 1H), 7.41 (d, J=2.0 Hz, 1H), 7.34 (d, J=3.2 Hz, 1H), 6.81 (d, J=1.7 Hz, 1H), 6.67 (d, J=3.2 Hz, 1H), 5.13 (s, 2H), 4.53-4.46 (m, 1H), 4.41-4.29 (m, 2H), 4.22-4.13 (m, 2H), 3.70-3.64 (m, 1H), 3.56-3.43 (m, 3H), 3.27-3.21 (m, 1H), 3.03 (d, J=13.0 Hz, 1H), 2.96 (d, J=12.7 Hz, 1H), 2.78-2.72 (m, 1H), 2.54-2.40 (m, 3H), 2.19-2.08 (m, 2H).

### Example 32

### Synthesis of 6,6-dimethyl-3-((4-(1-(((S)-morpholin-2-yl)methyl)-5-nitro-1H-indol-7-yl)pyrrolo[2,1-f][1,2,4]triazin-6-yl)methyl)-3-azabicyclo[3.1.0]hexane-2,4-dione 2,2,2-trifluoroacetate (32)

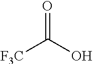

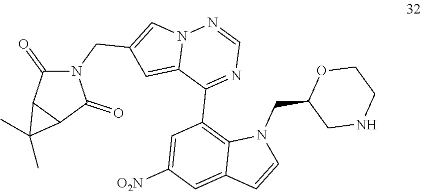 [SMILES: CC1(C)C2C(=O)N(Cc3cc4c(-c5cc([N+](=O)[O-])cc6ccn(C[C@@H]7CNCCO7)c56)ncnn4c3)C(=O)C21]

The title compound (32) was obtained as a TFA salt in 4% overall yield in a similar way to Example 5 with the exception that, in the first step of the synthesis, 7-bromo-5-nitro-1H-indole and tert-butyl (S)-2-(bromomethyl)morpholine-4-carboxylate were used instead of 7-bromo-5-(trifluoromethyl)-1H-indole and tert-butyl 3-((methylsulfonyl)oxy)pyrrolidine-1-carboxylate and to this reaction 0.36 equivalents of KI was added, and in the fourth step of the synthesis, the General Procedure IIIa was used instead of the General Procedure IIIb. The crude product was purified by preparative reversed-phase column chromatography (C-18, water+0.4‰ TFA/MeCN, 98:2 to 30:70, 30 min, 20 mL/min, Rₜ=19.4 min).

ESI-MS m/z for C₂₇H₂₈N₇O₅ found 530.4 [M+H]⁺; Rₜ=1.20 min (Method A); ¹H NMR (700 MHz, D₂O) δ 8.67 (s, 1H), 8.56 (s, 1H), 8.12 (s, 1H), 8.08 (s, 1H), 7.53-7.49 (m, 1H), 7.00-6.97 (m, 1H), 6.73 (s, 1H), 4.59 (s, 2H), 4.31-4.13 (m, 2H), 3.73-3.68 (m, 1H), 3.38-3.32 (m, 1H), 3.26-3.17 (m, 1H), 3.14-3.05 (m, 2H), 2.69-2.61 (m, 1H), 2.54-2.45 (m, 3H), 1.22 (s, 3H), 1.06 (s, 3H).

### Example 33

### Synthesis of 3-((4-(5-fluoro-1-(((S)-morpholin-2-yl)methyl)-1H-indol-7-yl)pyrrolo[2,1-f][1,2,4]triazin-6-yl)methyl)-6,6-dimethyl-3-azabicyclo[3.1.0]hexane-2,4-dione 2,2,2-trifluoroacetate (33)

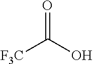

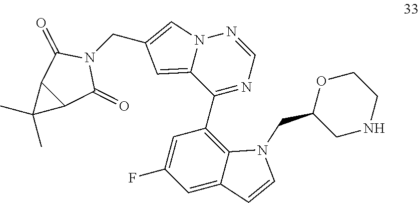 [SMILES: CC1(C)C2C(=O)N(Cc3cc4c(-c5cc(F)cc6ccn(C[C@@H]7CNCCO7)c56)ncnn4c3)C(=O)C21]

The title compound (33) was obtained as a TFA salt in 16% overall yield in a similar way to Example 5 with the exception that, in the first step of the synthesis, 7-bromo-5-fluoro-1H-indole and tert-butyl (S)-2-(bromomethyl)morpholine-4-carboxylate were used instead of 7-bromo-5-(trifluoromethyl)-1H-indole and tert-butyl 3-((methylsulfonyl)oxy)pyrrolidine-1-carboxylate and to this reaction 0.36 equivalents of KI was added, and in the fourth step of the synthesis, the General Procedure IIIa was used instead of the General Procedure IIIb. The crude product was purified by preparative reversed-phase column chromatography (C-18, water+0.4‰ TFA/MeCN, 98:2 to 30:70, 30 min, 20 mL/min, Rₜ=20.0 min).

ESI-MS m/z for C₂₇H₂₈FN₆O₃ found 503.3 [M+H]⁺; Rₜ=1.20 min (Method A); ¹H NMR (700 MHz, D₂O) δ 8.63 (s, 1H), 8.17 (s, 1H), 7.71-7.64 (m, 1H), 7.43-7.38 (m, 1H), 7.32-7.25 (m, 1H), 6.84 (s, 1H), 6.82-6.77 (m, 1H), 4.75-4.74 (m, 2H), 4.28-4.07 (m, 2H), 3.67-3.59 (m, 1H), 3.46-3.37 (m, 1H), 3.24-3.12 (m, 1H), 3.12-3.06 (m, 1H), 3.06-2.99 (m, 1H), 2.75-2.67 (m, 1H), 2.66-2.60 (m, 2H), 2.56-2.47 (m, 1H), 1.27 (s, 3H), 1.09 (s, 3H).

### Example 34

### Synthesis of methyl 7-(6-((6,6-dimethyl-2,4-dioxo-3-azabicyclo[3.1.0]hexan-3-yl)methyl)pyrrolo[2,1-f][1,2,4]triazin-4-yl)-1-(((S)-morpholin-2-yl)methyl)-1H-indole-5-carboxylate 2,2,2-trifluoroacetate (34)

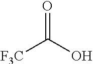

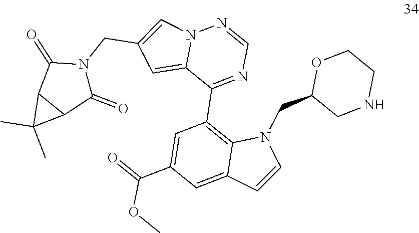 [SMILES: COC(=O)c1cc(-c2ncnn3cc(CN4C(=O)C5C(C4=O)C5(C)C)cc23)c2c(ccn2C[C@@H]2CNCCO2)c1]

The title compound (34) was obtained as a TFA salt in 1% overall yield in a similar way to Example 5 with the exception that, in the first step of the synthesis, methyl 7-bromo-1H-indole-5-carboxylate and tert-butyl (S)-2-(bromomethyl)morpholine-4-carboxylate were used instead of 7-bromo-5-(trifluoromethyl)-1H-indole and tert-butyl 3-((methylsulfonyl)oxy)pyrrolidine-1-carboxylate and to this reaction 0.36 equivalents of KI was added, and in the fourth step of the synthesis, the General Procedure IIIa was used instead of the General Procedure IIIb. The crude product was purified by preparative reversed-phase column chromatography (C-18, water+0.4‰ TFA/MeCN, 98:2 to 30:70, 30 min, 20 mL/min, Rₜ=17.9 min).

ESI-MS m/z for C₂₉H₃₁FN₆O₅ found 543.4 [M+H]⁺; Rₜ=1.13 min (Method A); ¹H NMR (700 MHz, D₂O) δ 8.58 (s, 1H), 8.57 (s, 1H), 8.14 (s, 1H), 7.96 (s, 1H), 7.45 (s, 1H), 6.96 (s, 1H), 6.73 (s, 1H), 4.65-4.60 (m, 2H), 4.31-4.09 (m, 2H), 3.87 (s, 3H), 3.69-3.63 (m, 1H), 3.43-3.34 (m, 1H), 3.24-3.13 (m, 1H), 3.10-3.03 (m, 2H), 2.71-2.60 (m, 1H), 2.57-2.44 (m, 3H), 1.24 (s, 3H), 1.07 (s, 3H).

### Example 35

### Synthesis of 6,6-dimethyl-3-((4-(1-(((S)-morpholin-2-yl)methyl)-5-(trifluoromethyl)-1H-indol-7-yl)pyrrolo[2,1-f][1,2,4]triazin-6-yl)methyl)-3-azabicyclo[3.1.0]hexane-2,4-dione 2,2,2-trifluoroacetate (35)

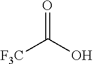

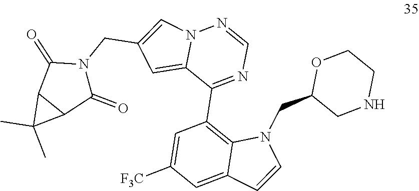 [SMILES: CC1(C)C2C(=O)N(Cc3cc4c(-c5cc(C(F)(F)F)cc6ccn(C[C@@H]7CNCCO7)c56)ncnn4c3)C(=O)C21]

The title compound (35) was obtained as a TFA salt in 3% overall yield in a similar way to Example 5 with the exception that, in the first step of the synthesis, 7-bromo-5-(trifluoromethyl)-1H-indole and tert-butyl (S)-2-(bromomethyl)morpholine-4-carboxylate were used instead of 7-bromo-5-(trifluoromethyl)-1H-indole and tert-butyl 3-((methylsulfonyl)oxy)pyrrolidine-1-carboxylate and to this reaction 0.36 equivalents of KI was added, and in the fourth step of the synthesis, the General Procedure IIIa was used instead of the General Procedure IIIb. The crude product was purified by preparative reversed-phase column chromatography (C-18, water+0.4‰ TFA/MeCN, 98:2 to 30:70, 30 min, 20 mL/min, Rₜ=21.6 min).

ESI-MS m/z for C₂₈H₂₈F₃N₆O₃ found 553.4 [M+H]⁺; Rₜ=1.35 min (Method A); ¹H NMR (700 MHz, D₂O) δ 8.61 (s, 1H), 8.30 (s, 1H), 8.16 (s, 1H), 7.71 (s, 1H), 7.49 (s, 1H), 6.95 (s, 1H), 6.78 (s, 1H), 4.73-4.68 (m, 2H), 4.38-4.12 (m, 2H), 3.70-3.64 (m, 1H), 3.44-3.34 (m, 1H), 3.25-3.14 (m, 1H), 3.11-3.03 (m, 2H), 2.72-2.63 (m, 1H), 2.63-2.57 (m, 2H), 2.54-2.45 (m, 1H), 1.26 (s, 3H), 1.07 (s, 3H).

### Example 36

### Synthesis of (S)-3-((4-(5-chloro-1-(morpholin-2-ylmethyl)-1H-indol-7-yl)pyrrolo[2,1-f][1,2,4]triazin-6-yl)methyl)-5,5-dimethylimidazolidine-2,4-dione hydrochloride (36)

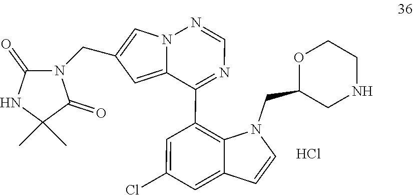 [SMILES: CC1(C)NC(=O)N(Cc2cc3c(-c4cc(Cl)cc5ccn(C[C@@H]6CNCCO6)c45)ncnn3c2)C1=O ; Cl]

### Step 1.

### Synthesis of 3-((4-chloropyrrolo[2,1-f][1,2,4]triazin-6-yl)methyl)-5,5-dimethylimidazolidine-2,4-dione (36a)

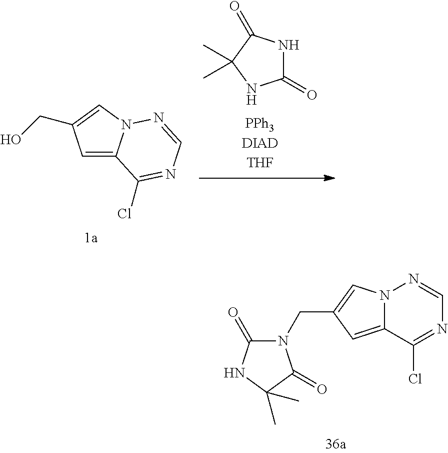

To a cooled to −10° C. solution of 1a (99 mg; 0.54 mmol), 5,5-dimethylimidazolidine-2,4-dione (83 mg; 0.65 mmol) and PPh₃ (170 mg; 0.65 mmol) in dry THE (1 mL) DIAD (138 μL; 0.70 mmol) was added. The resulting mixture was stirred at room temperature overnight. Then the reaction mixture was concentrated in vacuo and the residue was purified by flash column chromatography on silica (hexane/AcOEt, 100:0 to 0:100, v/v, 41 min, 20 mL/min). Compound 36a was obtained as a white crystals in 67% yield (105 mg; 0.36 mmol).

ESI-MS m/z for C₁₂H₁₃ClN₅O₂ found 294.0/296.0 [M+H]⁺; Rₜ=0.91 min (Method A) 6.43-6.39 (m, 1H), 4.80-4.73 (m, 1H), 4.44-4.35 (m, 1H), 4.07-3.77 (m, 4H), 3.46-3.40 (m, 1H), 2.97-2.88 (m, 1H), 2.66-2.56 (m, 1H), 1.44 (s, 9H).

### Step 2.

### Synthesis of tert-butyl (R)-2-((5-chloro-7-(6-((4,4-dimethyl-2,5-dioxoimidazolidin-1-yl)methyl)pyrrolo[2,1-f][1,2,4]triazin-4-yl)-1H-indol-1-yl)methyl)morpholine-4-carboxylate (36b)

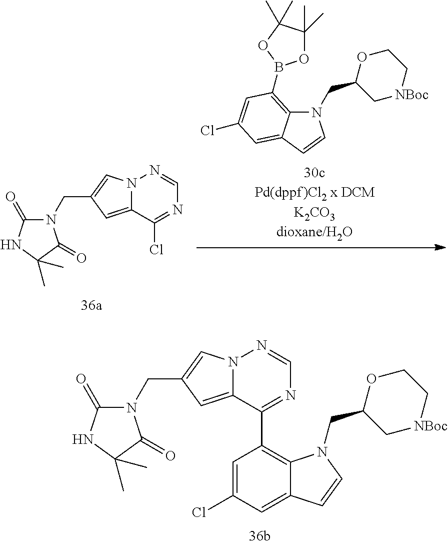 [SMILES: CC(C)(C)OC(=O)N1CCO[C@H](Cn2ccc3cc(Cl)cc(B4OC(C)(C)C(C)(C)O4)c32)C1 ; CC1(C)NC(=O)N(Cc2cc3c(Cl)ncnn3c2)C1=O ; CC(C)(C)OC(=O)N1CCO[C@H](Cn2ccc3cc(Cl)cc(-c4ncnn5cc(CN6C(=O)NC(C)(C)C6=O)cc45)c32)C1]

The title compound (36b) was obtained from 36a (76 mg; 0.259 mmol) and from 30c (289 mg; 0.311 mmol) according to the General Procedure IV with the exception that this reaction was carried out in 100° C. and then in 80° C. overnight. The crude product was purified by flash column chromatography on silica (hexane/AcOEt, 100:0 to 0:100, v/v, 30 min). Compound 36b was obtained in 46% yield (73 mg; 0.120 mmol).

ESI-MS m/z for C₃₀H₃₅ClN₇O₅ found 608.0/610.0 [M+H]⁺; Rₜ=1.62 min (Method A)

### Step 3.

### Synthesis of (S)-3-((4-(5-chloro-1-(morpholin-2-ylmethyl)-1H-indol-7-yl)pyrrolo[2,1-f][1,2,4]triazin-6-yl)methyl)-5,5-dimethylimidazolidine-2,4-dione hydrochloride (36)

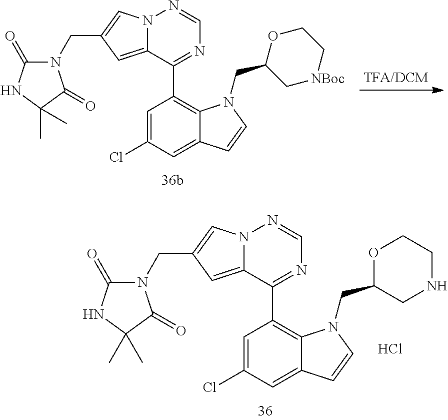 [SMILES: CC(C)(C)OC(=O)N1CCO[C@H](Cn2ccc3cc(Cl)cc(-c4ncnn5cc(CN6C(=O)NC(C)(C)C6=O)cc45)c32)C1 ; CC1(C)NC(=O)N(Cc2cc3c(-c4cc(Cl)cc5ccn(C[C@@H]6CNCCO6)c45)ncnn3c2)C1=O ; Cl]

The title compound (36) was obtained as a hydrochloride salt from 36b (73 mg; 0.120 mmol) according to the General Procedure IIIb in 82% yield (54 mg; 0.099 mmol). The crude product was purified by preparative reversed-phase column chromatography (C-18, water+0.3‰ HCl (36%)/MeCN, 99.9:0.1 to 45:55, 30 min, 20 mL/min, Rₜ=20.3 min).

ESI-MS m/z for C₂₅H₂₇ClN₇O₃ found 508.2/510.2 [M+H]⁺; Rₜ=1.15 min (Method A); ¹H NMR (700 MHz, D₂O) δ 8.67-8.58 (m, 1H), 8.17 (s, 1H), 8.00-7.91 (m, 1H), 7.52 (s, 1H), 7.44-7.35 (m, 1H), 6.85-6.73 (m, 2H), 4.86 (s, 2H), 4.35-4.10 (m, 2H), 3.70-3.56 (m, 1H), 3.37-3.26 (m, 1H), 3.24-3.11 (m, 1H), 3.11-2.98 (m, 2H), 2.70-2.55 (m, 1H), 2.55-2.39 (m, 1H), 1.43 (s, 6H).

### Example 37

### Synthesis of (S)-3-((4-(5-chloro-1-(morpholin-2-ylmethyl)-1H-indol-7-yl)pyrrolo[2,1-f][1,2,4]triazin-6-yl)methyl)imidazolidine-2,4-dione hydrochloride (37)

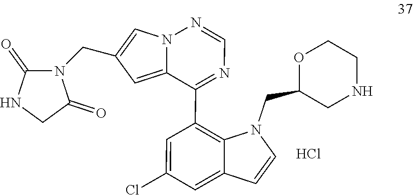 [SMILES: O=C1CNC(=O)N1Cc1cc2c(-c3cc(Cl)cc4ccn(C[C@@H]5CNCCO5)c34)ncnn2c1 ; Cl]

The title compound (37) was obtained as a hydrochloride salt in 17% overall yield in a similar way to Example 36 with the exception that, in the first step of the synthesis, imidazolidine-2,4-dione was used instead of 5,5-dimethylimidazolidine-2,4-dione. The crude product was purified by preparative reversed-phase column chromatography (C-18, water+0.3‰ HCl (36%)/MeCN, 99.9:0.1 to 45:55, 30 min, 20 mL/min, Rₜ=18.7 min).

ESI-MS m/z for C₂₃H₂₃ClN₇O₃ found 480.2/482.2 [M+H]⁺; Rₜ=1.03 min (Method A); ¹H NMR (700 MHz, D₂O) δ 8.69-8.54 (m, 1H), 8.20 (s, 1H), 7.97-7.88 (m, 1H), 7.43 (s, 1H), 7.40-7.36 (m, 1H), 6.90-6.83 (m, 1H), 6.80-6.72 (m, 1H), 4.85 (s, 2H), 4.29-4.10 (m, 2H), 4.07 (s, 2H), 3.67-3.55 (m, 1H), 3.43-3.32 (m, 1H), 3.24-3.10 (m, 1H), 3.09-2.98 (m, 2H), 2.72-2.59 (m, 1H), 2.54-2.42 (m, 1H).

### Example 38

### Synthesis of 3-((4-(5-chloro-2-((6,6-difluoro-1,4-oxazepan-2-yl)methyl)-3-methylphenyl)pyrrolo[2,1-f][1,2,4]triazin-6-yl)methyl)-6,6-dimethyl-3-azabicyclo[3.1.0]hexane-2,4-dione hydrochloride (38)

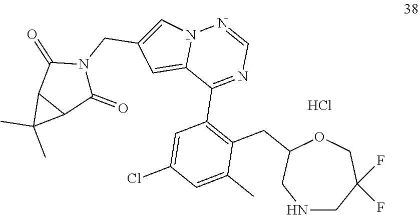 [SMILES: Cc1cc(Cl)cc(-c2ncnn3cc(CN4C(=O)C5C(C4=O)C5(C)C)cc23)c1CC1CNCC(F)(F)CO1 ; Cl]

### Step 1.

### Synthesis of 2,2-difluoro-3-((4-methoxybenzyl)amino)propan-1-ol (38a)

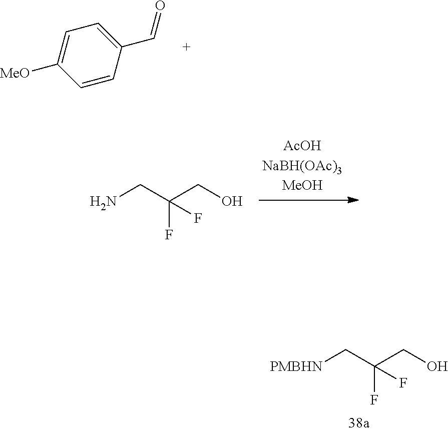

To the solution of 3-amino-2,2-difluoropropan-1-ol (1.00 g; 9.00 mmol) in MeOH (19 mL) 4-methoxybenzaldehyde (1.10 mL; 8.98 mmol) and glacial acetic acid (AcOH) (0.45 mL) were added and the mixture was stirred for 40 minutes at room temperature. Then sodium triacetoxyborohydride (NaBH(OAc)₃; 2.09 g; 9.88 mmol) was then added in several portions and this mixture was stirred at room temperature overnight. After this time MeOH was evaporated in vacuo. The crude product was purified by flash column chromatography on silica (DCM/MeOH, 100:0 to 50:50, v/v, 30 min, 20 mL/min). Compound 38a was obtained in 99% yield (2.05 g; 8.89 mmol).

ESI-MS m/z for C₁₁H₁₆F₂NO₂ found 232.0 [M+H]⁺; Rₜ=0.17 min (Method A); ¹H NMR (700 MHz, CDCl₃) δ 7.24-7.20 (m, 2H), 6.90-6.86 (m, 2H), 3.92-3.84 (m, 2H), 3.81 (s, 3H), 3.79 (s, 2H), 3.14-3.08 (m, 2H), 3.06 (s, 2H).

### Step 2.

### Synthesis of 6,6-difluoro-4-(4-methoxybenzyl)-1,4-oxazepan-3-one (38b)

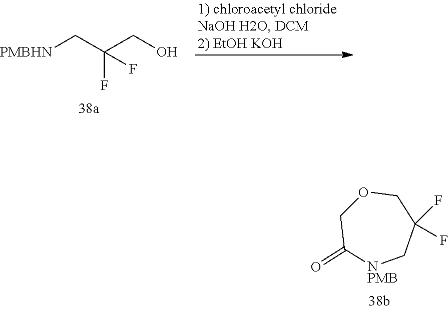

To the solution of 38a (1.00 g; 4.32 mmol) in DCM (5.84 mL) a solution of NaOH (173 mg; 4.32 mmol) in H₂O (3.5 mL) was added and the mixture was cooled to 0° C. Then to this solution chloroacetyl chloride (0.34 mL; 4.32 mmol) was added dropwise and the reaction mixture was then stirred at room temperature overnight. After this time, the phases were separated and an organic one was washed with 1 M NaOH, 1 M HCl and brine and then was dried over anhydrous MgSO₄, filtered and concentrated in vacuo. The residue was dissolved in EtOH (6 mL) and to this solution KOH (286 mg; 4.32 mmol) was added. The reaction mixture was stirred at room temperature overnight. Then the crude product was purified by flash column chromatography on silica (hexane/AcOEt, 100:0 to 0:100, v/v, 20 min, 30 mL/min). Compound 38b was obtained in 34% yield (0.40 g; 1.47 mmol).

ESI-MS m/z for C₁₃H₁₆F₂NO₃ found 272.0 [M+H]⁺; Rₜ=1.13 min (Method A); ¹H NMR (700 MHz, CDCl₃) δ 7.23-7.19 (m, 2H), 6.90-6.85 (m, 2H), 4.61 (s, 2H), 4.47 (s, 2H), 3.93 (t, J=12.5 Hz, 2H), 3.81 (s, 3H), 3.67 (t, J=11.4 Hz, 2H).

### Step 3.

### Synthesis of 2-(4-chloro-2-iodo-6-methylbenzyl)-6,6-difluoro-4-(4-methoxybenzyl)-1,4-oxazepan-3-one (38c)

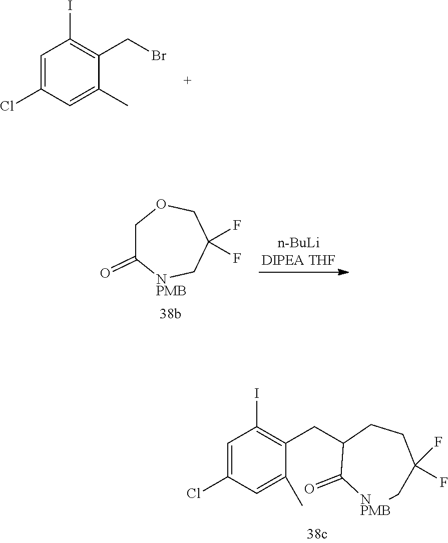

To the cooled to −78° C. solution of DIPEA (227 μL; 1.62 mmol) in dry THF (5.4 mL) n-BuLi (2.5 M in hexane; 648 μL; 1.62 mmol) was added dropwise and the resulting mixture was warmed to room temperature and stirred at this temperature for 15 minutes. Then the reaction mixture was cooled to −78° C. and to this mixture a solution of 38b (400 mg; 1.47 mmol) in dry THE (1.5 mL) was added and the mixture was stirred at this temperature for 1 hour. Then to this mixture a solution of 6b (507 mg; 1.47 mmol) in THE (1.5 mL) was added and a whole was allowed to warm to room temperature overnight. After this time, the reaction mixture was quenched with NH₄Cl and extracted with DCM (3×). The combined organic solutions were dried over anhydrous Na₂SO₄, filtered and concentrated in vacuo. Then the crude product was purified by flash column chromatography on silica (hexane/AcOEt, 100:0 to 80:20, v/v, 25 min, 20 mL/min). Compound 38c was obtained in 73% yield (572 mg; 1.07 mmol).

ESI-MS m/z for C₂₁H₂₂ClF₂INO₃ found 535.9/538.0 [M+H]⁺; Rₜ=2.01 min (Method A); ¹H NMR (700 MHz, CDCl₃) δ 7.71 (d, J=2.1 Hz, 1H), 7.25-7.22 (m, 2H), 7.16 (d, J=2.2 Hz, 1H), 6.92-6.88 (m, 2H), 4.65 (s, 2H), 4.59-4.55 (m, 1H), 4.07-4.00 (m, 1H), 3.93-3.83 (m, 1H), 3.82-3.81 (m, 3H), 3.62-3.50 (m, 2H), 3.46-3.38 (m, 1H), 3.37-3.29 (m, 1H), 2.40 (s, 3H).

### Step 4.

### Synthesis of 2-(4-chloro-2-iodo-6-methylbenzyl)-6,6-difluoro-4-(4-methoxybenzyl)-1,4-oxazepane (38d)

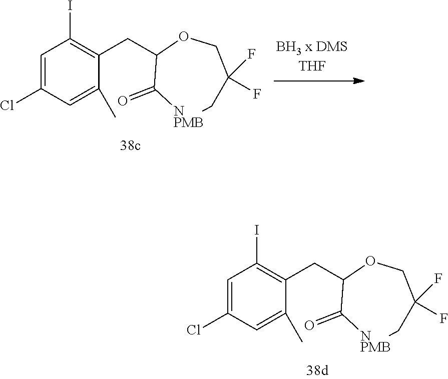

To the cooled to 0° C. solution of 38c (277 mg; 0.52 mmol) in dry THF (2.6 mL) BH₃×DMS (0.15 mL; 1.55 mmol) was added under an Argon atmosphere and this mixture was then stirred at 70° C. for 3 hours and then at room temperature overnight. The reaction progress was monitored by LC-MS. When analysis indicated completion of the reaction, the reaction mixture was cooled to 0° C. and quenched with MeOH and the solvents were evaporated in vacuo. The crude product was purified by flash column chromatography on silica (hexane/AcOEt, 100:0 to 70:30, v/v, 20 min, 18 mL/min). Compound 38d was obtained in 90% yield (245 mg; 0.47 mmol).

ESI-MS m/z for C₂₁H₂₄ClF₂INO₂ found 522.0/524.0 [M+H]⁺; Rₜ=2.28 min (Method A)

### Step 5.

### Synthesis of 2-(4-chloro-2-methyl-6-(4,4,5,5-tetramethyl-1,3,2-dioxaborolan-2-yl)benzyl)-6,6-difluoro-4-(4-methoxybenzyl)-1,4-oxazepane (38e)

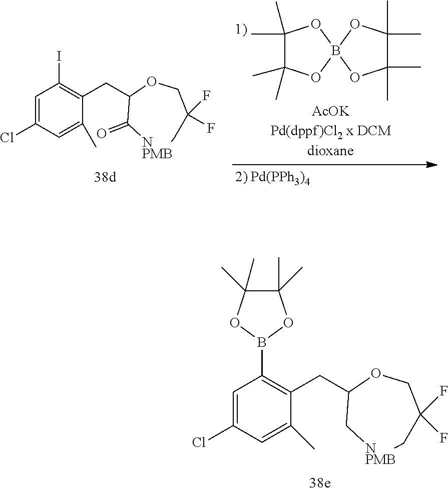

In a Schlenk flask was placed the compound 38d (237 mg; 0.450 mmol), bis(pinacolato)diboron (231 mg; 0.910 mmol), AcOK (133 mg; 1.360 mmol) and Pd(dppf)Cl₂×DCM (37 mg; 0.045 mmol) and dioxane (3.8 mL). The reaction mixture was intensively flushed with Ar. The mixture was capped and stirred at 90° C. overnight. Then Pd(PPh₃)₄ (23 mg; 0.020 mmol) and bis(pinacolato)diboron (231 mg; 0.910 mmol) were added and stirred at 90° C. overnight. Then another portion of bis(pinacolato)diboron (79 mg; 0.310 mmol) and Pd(dppf)Cl₂×DCM (41 mg; 0.045 mmol) were added and stirred at 90° C. for 4 hours. The reaction progress was monitored by LC-MS. When analysis indicated completion of the reaction, the reaction mixture was concentrated in vacuo and the crude product was purified by flash column chromatography on silica (hexane/AcOEt, 100:0 to 55:45, v/v, 25 min, 18 mL/min). Compound 38e was obtained in 99% yield (234 mg; 0.450 mmol).

ESI-MS m/z for C₂₇H₃₆BClF₂NO₄ found 522.1/524.1 [M+H]⁺; Rₜ=2.28 min (Method A)

### Step 6.

### Synthesis of 3-((4-(5-chloro-2-((6,6-difluoro-4-(4-methoxybenzyl)-1,4-oxazepan-2-yl)methyl)-3-methylphenyl)pyrrolo[2,1-f][1,2,4]triazin-6-yl)methyl)-6,6-dimethyl-3-azabicyclo[3.1.0]hexane-2,4-dione (38f)

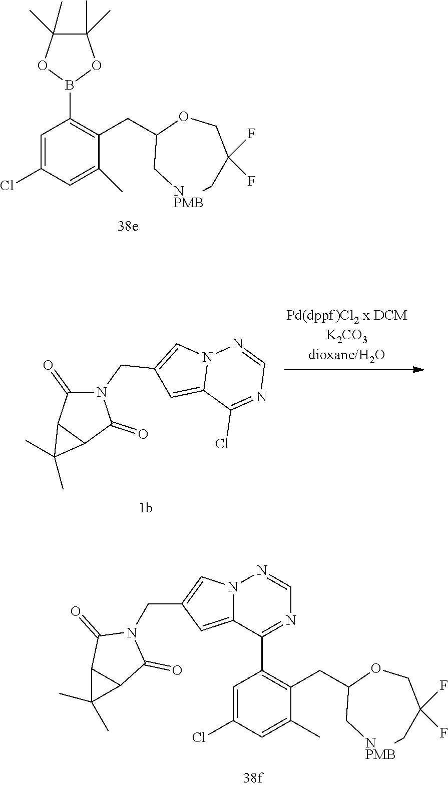

The title compound (38f) was obtained from 1b (137 mg; 0.450 mmol) and from 38e (234 mg; 0.450 mmol) according to the General Procedure IV in 31% yield (92 mg; 0.139 mmol). The crude product was purified by flash column chromatography on silica (hexane/AcOEt, 100:0 to 45:55, v/v, 35 min, 18 mL/min).

ESI-MS m/z for C₃₅H₃₇ClF₂N₅O₄ found 664.4/666.4 [M+H]⁻; Rₜ=2.04 min (Method A)

### Step 7.

### Synthesis of 3-((4-(5-chloro-2-((6,6-difluoro-1,4-oxazepan-2-yl)methyl)-3-methylphenyl)pyrrolo[2,1-f][1,2,4]triazin-6-yl)methyl)-6,6-dimethyl-3-azabicyclo[3.1.0]hexane-2,4-dione hydrochloride (38)

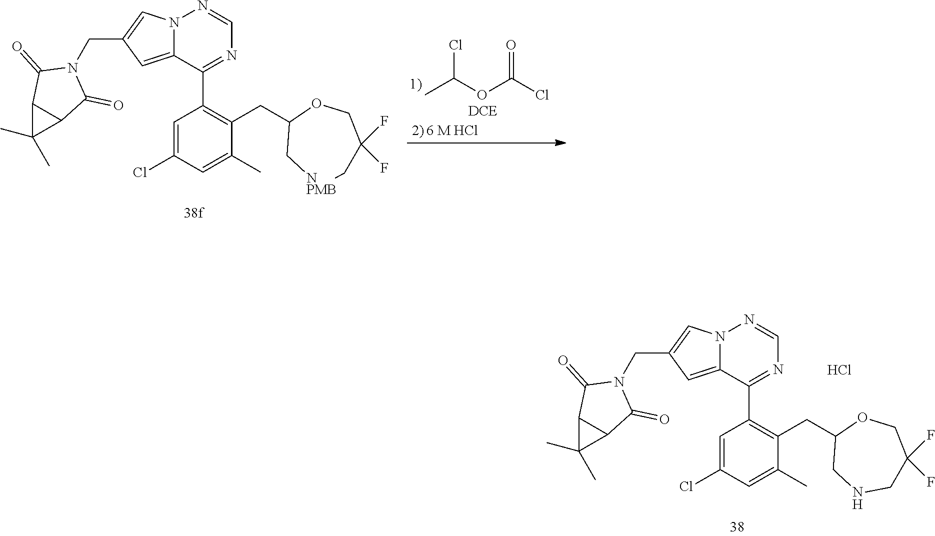

To the cooled to 0° C. solution of 38f (50 mg; 0.075 mmol) in DCE (0.3 mL) 1-chloroethyl carbonochloridate (32 μL; 0.300 mmol) was added dropwise and this mixture was then stirred at 80° C. overnight. The reaction progress was monitored by LC-MS. When analysis indicated completion of the reaction, the solvent was evaporated in vacuo and the residue was dissolved in 6 M HCl (1.5 mL) and whole was refluxed for 40 minutes. Then to this mixture water was added and a whole was purified by preparative reversed-phase column chromatography (C-18, water+0.3‰ HCl (36%)/MeCN, 99:1 to 15:85, 30 min, 20 mL/min, Rₜ=17.20 min). The title compound (38) was obtained as a hydrochloride salt in 8% yield (3.4 mg; 0.006 mmol).

ESI-MS m/z for C₂₇H₂₉ClF₂N₅O₃ found 544.4/546.4 [M+H]⁺; Rₜ=1.33 min (Method A); ¹H NMR (700 MHz, D₂O) δ 8.43 (s, 1H), 8.01 (s, 1H), 7.51-7.46 (m, 1H), 7.31-7.26 (m, 1H), 6.68 (s, 1H), 4.65-4.59 (m, 2H), 3.77-3.70 (m, 1H), 3.66-3.59 (m, 1H), 3.54-3.40 (m, 3H), 3.30-3.22 (m, 1H), 3.11-3.04 (m, 1H), 3.02-2.97 (m, 1H), 2.89-2.82 (m, 1H), 2.54-2.47 (m, 2H), 2.36 (s, 3H), 1.16 (s, 3H), 0.97 (s, 3H).

### Example 39

### Synthesis of (S)-3-((4-(5-chloro-1-(morpholin-2-ylmethyl)-1H-indol-7-yl)pyrrolo[2,1-f][1,2,4]triazin-6-yl)methyl)-1-methylpyrimidine-2,4(1H,3H)-dione hydrochloride (39)

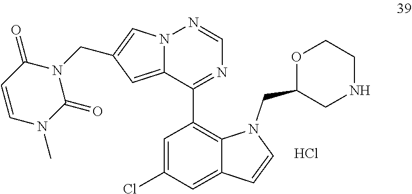 [SMILES: Cn1ccc(=O)n(Cc2cc3c(-c4cc(Cl)cc5ccn(C[C@@H]6CNCCO6)c45)ncnn3c2)c1=O ; Cl]

The title compound (39) was obtained as a hydrochloride salt in 6% overall yield in a similar way to Example 36 with the exception that, in the first step of the synthesis, 1-methylpyrimidine-2,4(1H,3H)-dione was used instead of 5,5-dimethylimidazolidine-2,4-dione. The crude product was purified by preparative reversed-phase column chromatography (C-18, water+0.30‰ HCl (36%)/MeCN, 99.9:0.1 to 50:50, 30 min, 20 mL/min, Rₜ=20.7 min).

ESI-MS m/z for C₂₅H₂₅ClN₇O₃ found 506.1/508.1 [M+H]⁺; Rₜ=1.10 min (Method A); ¹H NMR (700 MHz, D₂O) δ 8.59 (s, 1H), 8.21-8.16 (m, 1H), 7.90-7.83 (m, 1H), 7.54 (d, J=7.8 Hz, 1H), 7.39-7.34 (m, 1H), 7.33-7.28 (m, 1H), 6.90-6.83 (m, 1H), 6.77-6.72 (m, 1H), 5.82 (d, J=7.8 Hz, 1H), 5.25-5.18 (m, 2H), 4.26-4.03 (m, 2H), 3.65-3.55 (m, 1H), 3.40-3.33 (m, 1H), 3.31 (s, 3H), 3.21-3.08 (m, 1H), 3.09-2.98 (m, 2H), 2.72-2.55 (m, 1H), 2.54-2.41 (m, 1H).

### Example 40

### Synthesis of 3-((4-(5-chloro-3-methyl-2-(((5R)-5-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione hydrochloride (40)

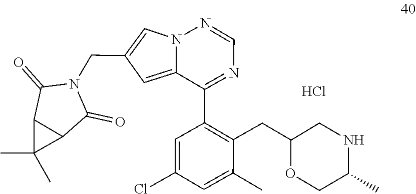

The title compound (40) was obtained as a single diastereoisomer as a hydrochloride salt in 1% overall yield in a similar way to Example 38 with the exception that, in the first step of the synthesis, (R)-2-aminopropan-1-ol was used instead of 3-amino-2,2-difluoropropan-1-ol and in the last step of the synthesis, MeOH was used instead of 6 M HCl. In the fourth step of the synthesis, two diastereoisomers were separated and then the reaction was carried on with one of them. The crude product was purified by preparative reversed-phase column chromatography (column: Cosmosil Cholester 20×250 mm, water+0.3‰ HCl (36%)/MeCN, 99:1 to 20:80, 30 min, 20 mL/min, Rₜ=16.25 min).

ESI-MS m/z for C₂₇H₃₁ClN₅O₃ found 508.5/510.5 [M+H]⁺; Rₜ=1.22 min (Method A); ¹H NMR (700 MHz, Methanol-d₄) δ 8.62 (s, 1H), 8.33-8.27 (m, 1H), 7.58-7.53 (m, 1H), 7.42-7.39 (m, 1H), 6.95-6.90 (m, 1H), 4.71-4.67 (m, 2H), 3.75-3.68 (m, 2H), 3.28-3.18 (m, 2H), 3.17-3.10 (m, 1H), 3.00-2.96 (m, 1H), 2.92-2.86 (m, 1H), 2.84-2.78 (m, 1H), 2.50 (s, 3H), 2.47 (s, 2H), 1.24 (s, 3H), 1.14 (d, J=6.5 Hz, 3H), 1.09 (s, 3H).

### Example 41

### Synthesis of 3-((4-(5-chloro-3-methyl-2-(((5R)-5-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione hydrochloride (41)

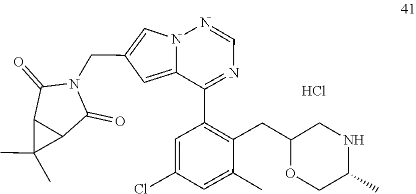

The title compound (41) was obtained as a second single diastereoisomer as a hydrochloride salt in 0.3% overall yield in the same synthesis like the compound 40. In the fourth step of the synthesis, two diastereoisomers were separated and then the reaction was carried on with one of them. The crude product was purified by preparative reversed-phase column chromatography (column: Cosmosil Cholester 20×250 mm, water+0.3‰ HCl (36%)/MeCN, 99:1 to 20:80, 30 min, 20 mL/min, Rₜ=16.10 min).

ESI-MS m/z for C₂₇H₃₁ClN₅O₃ found 508.4/510.4 [M+H]⁺; Rₜ=1.24 min (Method A); ¹H NMR (700 MHz, Methanol-d₄) δ 8.61 (s, 1H), 8.27-8.24 (m, 1H), 7.55-7.53 (m, 1H), 7.41-7.38 (m, 1H), 6.86-6.84 (m, 1H), 4.70-4.68 (m, 2H), 3.77-3.72 (m, 1H), 3.60-3.55 (m, 1H), 3.47-3.43 (m, 1H), 3.41-3.37 (m, 1H), 3.04-2.94 (m, 4H), 2.50 (s, 3H), 2.47 (s, 2H), 1.24 (s, 3H), 1.21 (d, J=6.9 Hz, 3H), 1.09 (s, 3H).

### Example 42

### Synthesis of 3-((4-(5-chloro-2-((6-(fluoromethyl)morpholin-2-yl)methyl)-3-methylphenyl)pyrrolo[2,1-f][1,2,4]triazin-6-yl)methyl)-6,6-dimethyl-3-azabicyclo[3.1.0]hexane-2,4-dione hydrochloride (42)

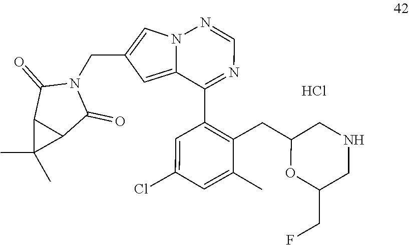 [SMILES: Cc1cc(Cl)cc(-c2ncnn3cc(CN4C(=O)C5C(C4=O)C5(C)C)cc23)c1CC1CNCC(CF)O1 ; Cl]

### Step 1.

### Synthesis of 2-allyl-1-bromo-5-chloro-3-methylbenzene (42a)

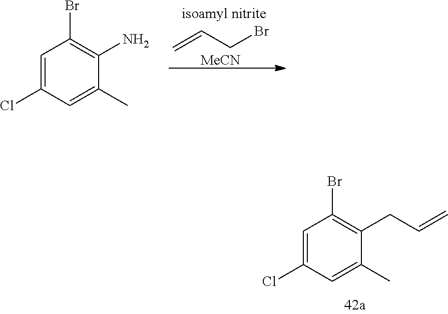

To the cooled to −5° C. solution of isoamyl nitrite (8.94 mL; 67.87 mmol) and allyl bromide (51.6 mL; 67.86 mmol) in dry MeCN (50 mL) a solution of 2-bromo-4-chloro-6-methylaniline (10 g; 45.24 mmol) in dry MeCN (25 mL) was added dropwise. The cooling bath was removed and the mixture was then stirred at room temperature overnight. The reaction progress was monitored by LC-MS. When analysis indicated completion of the reaction, the solvent was removed in vacuo. The crude product was purified by flash column chromatography on silica (column: 220 g; hexane/AcOEt, 100:0 to 75:25, v/v, 20 min, 215 mL/min). Compound 42a was obtained in 44% yield (4.88 g; 19.87 mmol).

### Step 2.

### Synthesis of 2-(2-bromo-4-chloro-6-methylbenzyl)oxirane (42b)

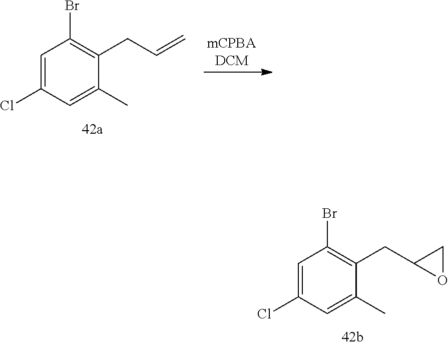

To the solution of 42a (4.88 g; 19.87 mmol) in DCM (40 mL) mCPBA (7.35 g; 29.81 mmol; 70%) was added dropwise and the mixture was then stirred at room temperature overnight. The reaction progress was monitored by LC-MS. When analysis indicated completion of the reaction, the mixture was diluted with DCM and washed with 5% NaHCO₃aq. The organic layer was dried over anhydrous MgSO₄, filtered and concentrated in vacuo. The crude product was purified by flash column chromatography on silica (hexane/AcOEt, 100:0 to 90:10, v/v, 20 min, 60 mL/min). Compound 42b was obtained as a colorless oil in 68% yield (3.50 g; 13.46 mmol).

### Step 3.

### Synthesis of 1-amino-3(2-bromo-4-chloro-6-methylphenyl)propan-2-ol (42c)

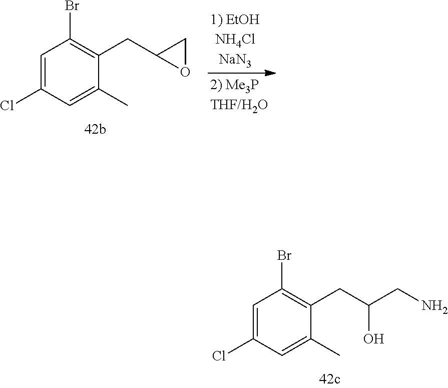

To the solution of 42b (3.38 g; 12.93 mmol) in EtOH (50 mL) NH₄Cl (2.07 g; 38.78 mmol) and NaN₃ (2.52 g; 38.78 mmol) were added and a whole was then stirred at 90° C. overnight. The reaction progress was monitored by TLC. When analysis indicated completion of the reaction, the solvent was removed in vacuo and the residue was redissolved in AcOEt and this solution was washed with water. The organic layer was dried over anhydrous MgSO₄, filtered and concentrated in vacuo. The residue was dissolved in THF/water (100 mL/10 mL; 10:1, v/v). Then to this solution Me₃P (1 M in THF; 36.47 mL; 36.45 mmol) was added and a whole was stirred at room temperature overnight. When analysis indicated completion of the reaction, the solvent was removed in vacuo and the residue was washed with Et₂O. The crude product was used to the next step without any additional purification.

ESI-MS m/z for C₁₀H₁₄BrClNO found 277.8/279.8 [M+H]⁻; Rₜ=0.88 min (Method A)

### Step 4.

### Synthesis of (R)-2-bromo-3-(tert-butoxy)propanoic acid (42d)

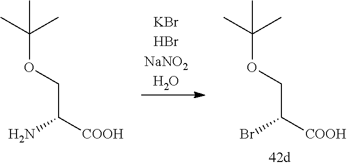

To the solution of O-(tert-butyl)-D-serine (80 g; 496 mmol) and KBr (206.6 g; 1740 mmol) in water (370 mL) HBr (48%; 133 mL) was added at room temperature and a whole was then cooled to −20° C. and to this mixture NaNO₂ (41.1 g; 595 mmol) was slowly added over a period of 1.5 hours (the temperature was kept between −15° C. and −5° C.). Then the mixture was stirred at −5° C. The reaction progress was monitored by LC-MS. When analysis indicated completion of the reaction, the product was isolated by extraction with Et₂O (3×300 mL). The combined organic layers were washed with brine (200 mL) and then dried over anhydrous Na₂SO₄, filtered and concentrated in vacuo. Compound 42d was obtained in 91% yield (101.95 g; 453 mmol).

ESI-MS m/z for C₇H₁₂BrO₃ found 223.0/225.0 [M−H]; Rₜ=1.04 min (Method A)

### Step 5.

### Synthesis of (2R)-2-bromo-N-(3-(2-bromo-4-chloro-6-methylphenyl)-2-hydroxypropyl)-3-(tert-butoxy)propanamide (42e)

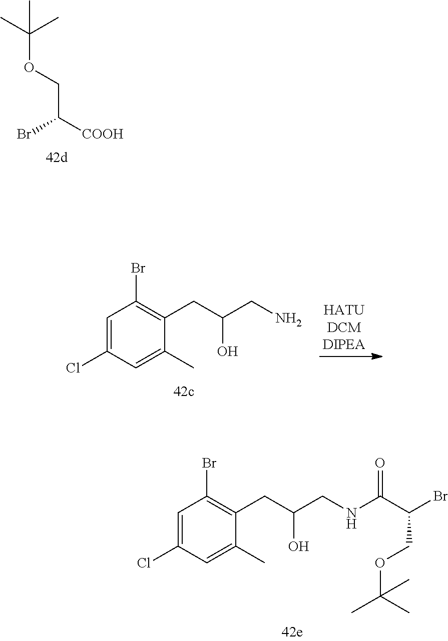 [SMILES: CC(C)(C)OC[C@@H](Br)C(=O)O ; Cc1cc(Cl)cc(Br)c1CC(O)CN ; Cc1cc(Cl)cc(Br)c1CC(O)CNC(=O)[C@H](Br)COC(C)(C)C]

The title compound (42e) was obtained from 42c (1.87 g; 6.73 mmol) and from 42d (1.51 g; 6.73 mmol) according to the General Procedure I in 99% yield (3.22 g; 6.66 mmol). The crude product was used to the next step without any additional purification.

ESI-MS m/z for C₁₇H₂₅Br₂ClNO₃ found 488.9/487.9 [M+H]⁺; Rₜ=1.65 min (Method A)

### Step 6.

### Synthesis of 6-(2-bromo-4-chloro-6-methylbenzyl)-2-(tert-butoxymethyl)morpholin-3-one (42f)

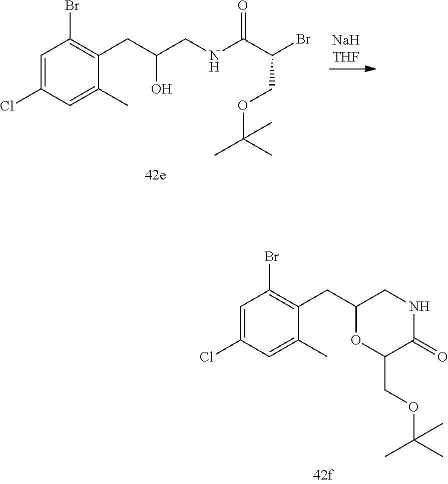 [SMILES: Cc1cc(Cl)cc(Br)c1CC(O)CNC(=O)[C@H](Br)COC(C)(C)C ; Cc1cc(Cl)cc(Br)c1CC1CNC(=O)C(COC(C)(C)C)O1]

To the solution of 42e (3.22 g; 6.66 mmol) in dry THF (40 mL) NaH (60% in mineral oil; 323 mg; 8.07 mmol) was added and stirred at room temperature for 1 hour. The reaction progress was monitored by LC-MS. When analysis indicated completion of the reaction, to the reaction mixture 2 M HCl (30 mL) was carefully added and a product was extracted with AcOEt (2×). The combined organic fractions were dried over anhydrous MgSO₄, filtered and concentrated in vacuo. Compound 42f was obtained in 79% yield (2.13 g; 5.28 mmol).

ESI-MS m/z for C₁₇H₂₄BrClNO₃ found 403.9/405.9 [M+H]⁺; Rₜ=1.64 min (Method A)

### Step 7.

### Synthesis of (6-(2-bromo-4-chloro-6-methylbenzyl)morpholin-2-yl)methanol (42g)

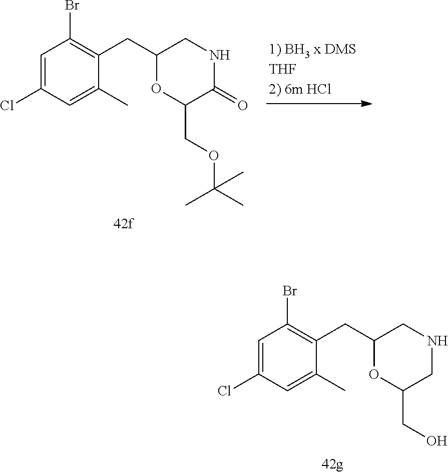 [SMILES: Cc1cc(Cl)cc(Br)c1CC1CNC(=O)C(COC(C)(C)C)O1 ; Cc1cc(Cl)cc(Br)c1CC1CNCC(CO)O1]

To the solution of 42f (2.13 g; 5.28 mmol) in dry THF (26.4 mL) BH₃×DMS (10 mL; 10.54 mmol) was added under an Argon atmosphere and this mixture was then refluxed for 4 hours. The reaction progress was monitored by LC-MS. When analysis indicated completion of the reaction, the reaction mixture was cooled to room temperature and to this mixture 6 M HCl (20 mL) was added dropwise. The whole was refluxed for 4 hours. Then the solvents were removed in vacuo. The crude product was used to the next step without any additional purification.

ESI-MS m/z for C₁₃H₁₈BrClNO₂ found 333.9/335.9 [M+H]⁺; Rₜ=0.93 min (Method A)

### Step 8.

### Synthesis of tert-butyl 2-(2-bromo-4-chloro-6-methylbenzyl)-6-(hydroxymethyl)morpholine-4-carboxylate (42h)

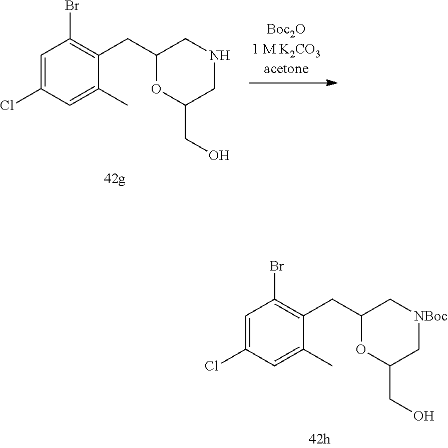 [SMILES: Cc1cc(Cl)cc(Br)c1CC1CNCC(CO)O1 ; Cc1cc(Cl)cc(Br)c1CC1CN(C(=O)OC(C)(C)C)CC(CO)O1]

To the suspension of 42g (the crude product) in 1 M K₂CO₃ (40 mL) a solution of Boc₂O (1.72 g; 7.90 mmol) in acetone (40 mL) was added and a whole was stirred at room temperature for 4 hours. The reaction progress was monitored by LC-MS. When analysis indicated completion of the reaction, acetone was removed in vacuo and the residue was extracted with AcOEt (2×). The combined organic layers were dried over anhydrous MgSO₄, filtered and concentrated in vacuo. The crude product was purified by flash column chromatography on silica (hexane/AcOEt, 100:0 to 65:35, v/v, 30 min, 60 mL/min). Compound 42h was obtained as a colorless oil in 71% yield (per two steps)(1.62 g; 3.74 mmol).

ESI-MS m/z for C₁₃H₁₈BrClNO₂ found 333.9/335.9 [M+H-Boc]⁺; Rₜ=1.71 min (Method A); ¹H NMR (700 MHz, CDCl₃) δ 7.43 (d, J=2.2 Hz, 1H), 7.12 (d, J=2.2 Hz, 1H), 4.07-3.88 (m, 2H), 3.75-3.54 (m, 3H), 3.49-3.41 (m, 2H), 3.20-3.12 (m, 1H), 3.04-2.93 (m, 2H), 2.38 (s, 3H), 1.45 (s, 9H).

### Step 9.

### Synthesis of tert-butyl 2-(2-bromo-4-chloro-6-methylbenzyl)-6-(((methylsulfonyl)oxy)methyl)morpholine-4-carboxylate (42i)

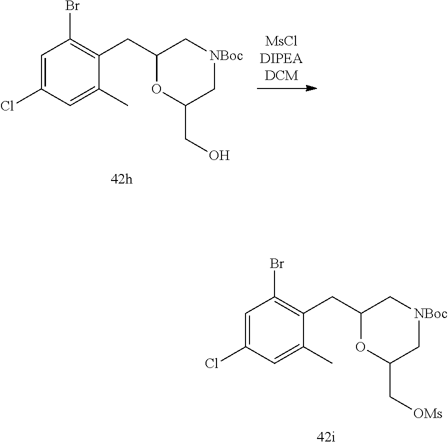 [SMILES: Cc1cc(Cl)cc(Br)c1CC1CN(C(=O)OC(C)(C)C)CC(CO)O1 ; Cc1cc(Cl)cc(Br)c1CC1CN(C(=O)OC(C)(C)C)CC(COS(C)(=O)=O)O1]

To the solution of 42h (0.70 g; 1.61 mmol) in dry DCM (12.88 mL) DIPEA (0.42 mL; 2.41 mmol) was added and the solution was cooled to 0° C. To this mixture MsCl (0.14 mL; 1.77 mmol) was added dropwise and then the cooling bath was removed and the resulting mixture was then stirred at room temperature for 1 hour. The reaction progress was monitored by LC-MS. When analysis indicated completion of the reaction, the reaction mixture was diluted with DCM and washed with 5% NaHCO₃. The organic layer was dried over anhydrous MgSO₄, filtered and concentrated in vacuo. Compound 42i was obtained in 99% yield (814 mg; 1.59 mmol).

ESI-MS m/z for C₁₉H₂₈BrClNO₆S found 512.0/514.0 [M+H]⁺; Rₜ=1.86 min (Method A)

### Step 10.

### Synthesis of tert-butyl 2-(2-bromo-4-chloro-6-methylbenzyl)-6-(fluoromethyl)morpholine-4-carboxylate (42j)

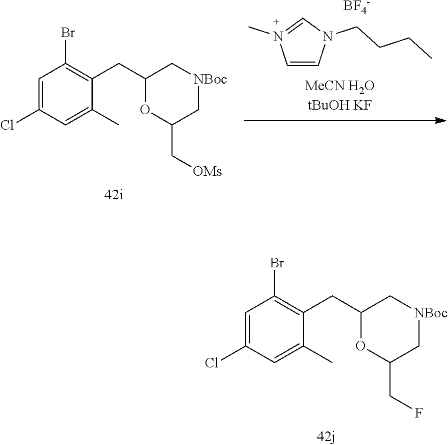

To the solution of 42i (814 mg; 1.59 mmol) in MeCN (4.8 mL) H₂O (0.14 mL; 8.00 mmol), BMIM tetrafluoroborate (2.4 mL) and KF (465 mg; 8.00 mmol) were added and a whole was stirred at 100° C. in a sealed tube for 2 hours. The reaction progress was monitored by LC-MS. Then to this mixture another portion of BMIM tetrafluoroborate (3 mL) and tBuOH (5 mL) were added and a whole was stirred at 100° C. in a sealed tube for 10 hours. When analysis indicated completion of the reaction, to the reaction mixture water (80 mL) was added and the product was extracted with Et₂O (5×). The combined organic layers were dried over anhydrous MgSO₄, filtered and concentrated in vacuo. The residue was purified by flash column chromatography on silica (hexane/AcOEt, 100:0 to 80:20, v/v, 50 min, 40 mL/min). Compound 42j was obtained in 49% yield (340 mg; 0.78 mmol).

ESI-MS m/z for C₁₈H₂₅BrClFNO₃ found 436.0/438.0 [M+H]⁺; Rₜ=2.10 min (Method A); ¹H NMR (700 MHz, CDCl₃) δ 7.43 (s, 1H), 7.11 (s, 1H), 4.55-4.37 (m, 2H), 4.13-3.98 (m, 2H), 3.73-3.38 (m, 3H), 3.21-2.95 (m, 3H), 2.37 (s, 3H), 1.46 (s, 9H).

### Step 11.

### Synthesis of tert-butyl 2-(4-chloro-2-methyl-6-(4,4,5,5-tetramethyl-1,3,2-dioxaborolan-2-yl)benzyl)-6-(fluoromethyl)morpholine-4-carboxylate (42k)

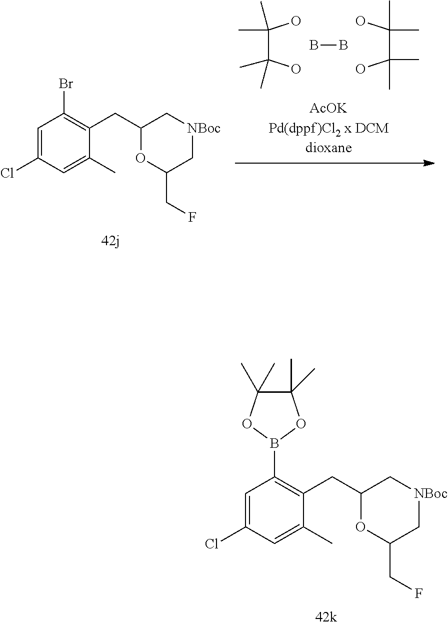

In a sealed tube was placed the compound 42j (170 mg; 0.389 mmol), bis(pinacolato)diboron (148 mg; 0.584 mmol), AcOK (115 mg; 1.167 mmol) and Pd(dppf)Cl₂×DCM (31.7 mg; 0.039 mmol) and dioxane (5 mL). The reaction mixture was intensively flushed with Ar. The mixture was capped and stirred at 110° C. overnight. The reaction progress was monitored by LC-MS. When analysis indicated completion of the reaction, the reaction mixture was concentaded in vacuo and the crude product was purified by flash column chromatography on silica (hexane/AcOEt, 100:0 to 0:100, v/v, 30 min, 13 mL/min). Compound 42k was obtained in 90% yield (170 mg; 0.352 mmol; 50% purity).

ESI-MS m/z for C₁₉H₂₉BClFNO₃ found 384.0/386.0 [M+H-Boc]⁺; Rₜ=2.21 min (Method A)

### Step 12.

### Synthesis of tert-butyl 2-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylbenzyl)-6-(fluoromethyl)morpholine-4-carboxylate (42l)

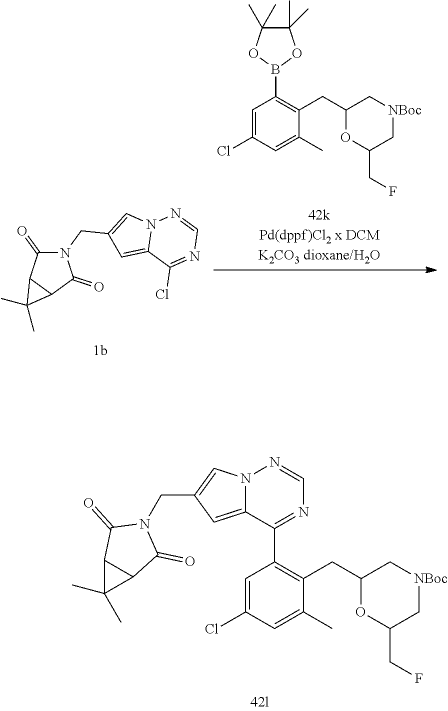 [SMILES: Cc1cc(Cl)cc(B2OC(C)(C)C(C)(C)O2)c1CC1CN(C(=O)OC(C)(C)C)CC(CF)O1 ; CC1(C)C2C(=O)N(Cc3cc4c(Cl)ncnn4c3)C(=O)C21 ; Cc1cc(Cl)cc(-c2ncnn3cc(CN4C(=O)C5C(C4=O)C5(C)C)cc23)c1CC1CN(C(=O)OC(C)(C)C)CC(CF)O1]

The title compound (42l) was obtained from 1b (107 mg; 0.351 mmol) and from 42k (170 mg; 0.352 mmol; 50% purity) according to the General Procedure IV in 14% yield (30 mg; 0.048 mmol). The crude product was purified by flash column chromatography on silica (hexane/AcOEt, 100:0 to 40:60, v/v, 35 min, 13 mL/min).

ESI-MS m/z for C₃₂H₃₈ClFN₅O₅ found 626.2/628.2 [M+H]⁺; Rₜ=1.78 min (Method A)

### Step 13.

### Synthesis of 3-((4-(5-chloro-2-((6-(fluoromethyl)morpholin-2-yl)methyl)-3-methylphenyl)pyrrolo[2,1-f][1,2,4]triazin-6-yl)methyl)-6,6-dimethyl-3-azabicyclo[3.1.0]hexane-2,4-dione hydrochloride (42)

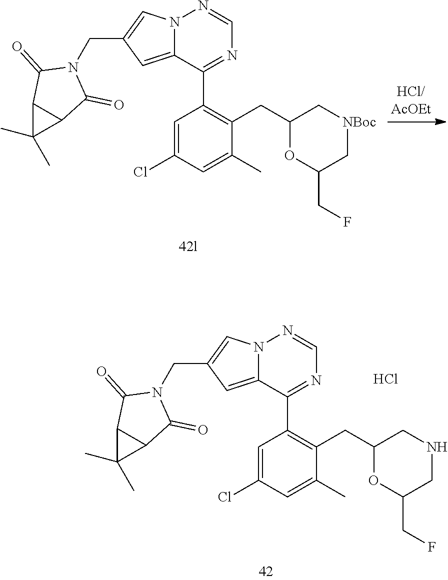 [SMILES: Cc1cc(Cl)cc(-c2ncnn3cc(CN4C(=O)C5C(C4=O)C5(C)C)cc23)c1CC1CN(C(=O)OC(C)(C)C)CC(CF)O1 ; Cc1cc(Cl)cc(-c2ncnn3cc(CN4C(=O)C5C(C4=O)C5(C)C)cc23)c1CC1CNCC(CF)O1 ; Cl]

The title compound (42) was obtained as a hydrochloride salt from 42l (20 mg; 0.032 mmol) according to the General Procedure IIIa in 59% yield (10.5 mg; 0.019 mmol). The crude product was purified by preparative reversed-phase column chromatography (column: Cosmosil Cholester 20×250 mm, water+0.3‰ HCl (36%)/MeCN, 98:2 to 40:60, 30 min, 20 mL/min, Rₜ=19.8 min).

ESI-MS m/z for C₂₇H₃₀ClFN₅O₃ found 526.5/528.5 [M+H]⁺; Rₜ=1.24 min (Method A); ¹H NMR (700 MHz, Methanol-d₄) δ 8.53 (s, 1H), 8.07 (d, J=1.3 Hz, 1H), 7.50 (d, J=2.1 Hz, 1H), 7.36 (d, J=2.2 Hz, 1H), 6.68 (d, J=1.2 Hz, 1H), 4.68-4.64 (m, 2H), 4.55-4.44 (m, 1H), 4.42-4.32 (m, 1H), 4.16-4.11 (m, 1H), 3.90-3.82 (m, 1H), 3.28-3.24 (m, 1H), 3.23-3.18 (m, 1H), 3.18-3.14 (m, 1H), 3.11-3.05 (m, 1H), 3.02-2.98 (m, 1H), 2.92-2.87 (m, 1H), 2.48 (s, 3H), 2.45 (s, 2H), 1.23 (s, 3H), 1.08 (s, 3H); ¹⁹F NMR (376 MHz, Methanol-d₄) δ −228.32.

### Example 43

### Synthesis of 3-((4-(5-chloro-2-((6-(fluoromethyl)morpholin-2-yl)methyl)-3-methylphenyl)pyrrolo[2,1-f][1,2,4]triazin-6-yl)methyl)-6,6-dimethyl-3-azabicyclo[3.1.0]hexane-2,4-dione hydrochloride (43)

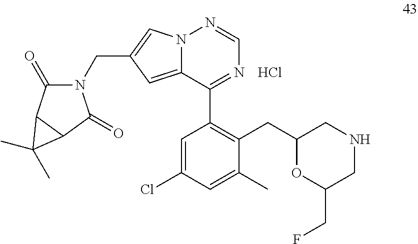 [SMILES: Cc1cc(Cl)cc(-c2ncnn3cc(CN4C(=O)C5C(C4=O)C5(C)C)cc23)c1CC1CNCC(CF)O1 ; Cl]

The title compound (43) was obtained as a single isomer (opposite to the compound 42) as a hydrochloride salt in 3% overall yield in a similar way to Example 42 with the exception that, in the fourth step of the synthesis, O-(tert-butyl)-L-serine was used instead of O-(tert-butyl)-D-serine and in the last step of the synthesis, the crude product was purified by preparative reversed-phase column chromatography (column: Cosmosil Cholester 20×250 mm, water+0.3‰ HCl (36%)/MeCN, 98:2 to 40:60, 30 min, 20 mL/min, Rₜ=19.8 min).

ESI-MS m/z for C₂₇H₃₀ClFN₅O₃ found 526.4/528.4 [M+H]⁺; Rₜ=1.26 min (Method A); ¹H NMR (700 MHz, Methanol-d₄) δ 8.50 (s, 1H), 8.03 (d, J=1.4 Hz, 1H), 7.47 (d, J=2.1 Hz, 1H), 7.33 (d, J=2.2 Hz, 1H), 6.62 (d, J=1.4 Hz, 1H), 4.66-4.61 (m, 2H), 4.56-4.25 (m, 2H), 4.15-4.07 (m, 1H), 3.90-3.77 (m, 1H), 3.26-3.15 (m, 2H), 3.15-3.04 (m, 2H), 3.00-2.94 (m, 1H), 2.91-2.83 (m, 1H), 2.45 (s, 3H), 2.43 (s, 2H), 1.20 (s, 3H), 1.05 (s, 3H); ¹⁹F NMR (376 MHz, Methanol-d₄) δ −228.35.

### Example 44

### Synthesis of 3-((4-(5-chloro-1-(((S)-morpholin-2-yl)methyl)-1H-indazol-7-yl)pyrrolo[2,1-f][1,2,4]triazin-6-yl)methyl)-6,6-dimethyl-3-azabicyclo[3.1.0]hexane-2,4-dione 2,2,2-trifluoroacetate (44)

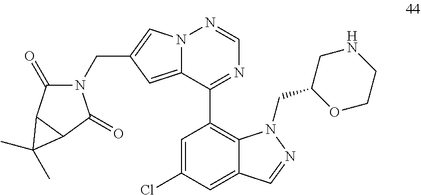 [SMILES: CC1(C)C2C(=O)N(Cc3cc4c(-c5cc(Cl)cc6cnn(C[C@@H]7CNCCO7)c56)ncnn4c3)C(=O)C21]

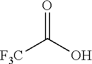

The title compound (44) was obtained as a TFA salt in 2% overall yield in a similar way to Example 5 with the exception that, in the first step of the synthesis, 7-Bromo-5-chloro-1H-indazole and tert-butyl (S)-2-(bromomethyl)morpholine-4-carboxylate were used instead of 7-bromo-5-(trifluoromethyl)-1H-indole and tert-butyl 3-((methylsulfonyl)oxy)pyrrolidine-1-carboxylate and to this reaction 0.36 equivalents of KI was added (in this step of the synthesis two tautomeric compounds were formed), and in the fourth step of the synthesis, the General Procedure IIIa was used instead of the General Procedure IIIb. The crude product was purified by preparative reversed-phase column chromatography (C-18, water+0.4‰ TFA/MeCN, 98:2 to 30:70, 30 min, 20 mL/min, Rₜ=19.3 min).

ESI-MS m/z for C₂₆H₂₇ClN₇O₃ found 520.2/522.2 [M+H]⁺; Rₜ=1.19 min (Method A); ¹H NMR (700 MHz, D₂O) δ 8.63 (s, 1H), 8.34 (s, 1H), 8.18 (d, J=1.5 Hz, 1H), 8.16 (d, J=2.0 Hz, 1H), 7.80 (d, J=2.0 Hz, 1H), 6.89 (s, 1H), 4.75 (s, 2H), 4.64-4.57 (m, 1H), 4.49-4.44 (m, 1H), 3.79-3.73 (m, 1H), 3.26-3.18 (m, 2H), 3.17-3.12 (m, 1H), 3.05-2.99 (m, 1H), 2.63 (s, 2H), 2.60-2.53 (m, 1H), 2.45-2.38 (m, 1H), 1.26 (s, 3H), 1.09 (s, 3H).

### Example 45

### Synthesis of 3-((4-(5-chloro-3-fluoro-1-(((S)-morpholin-2-yl)methyl)-1H-indazol-7-yl)pyrrolo[2,1-f][1,2,4]triazin-6-yl)methyl)-6,6-dimethyl-3-azabicyclo[3.1.0]hexane-2,4-dione hydrochloride (45)

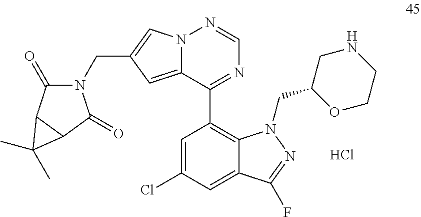 [SMILES: CC1(C)C2C(=O)N(Cc3cc4c(-c5cc(Cl)cc6c(F)nn(C[C@@H]7CNCCO7)c56)ncnn4c3)C(=O)C21 ; Cl]

### Step 1.

### Synthesis of 7-bromo-5-chloro-3-fluoro-1H-indazole (45a)

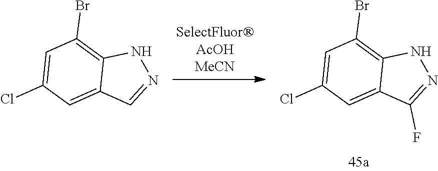 [SMILES: Clc1cc(Br)c2[nH]ncc2c1 ; Fc1n[nH]c2c(Br)cc(Cl)cc12]

To the solution of 7-bromo-5-chloro-1H-indazole (1.20 g; 5.22 mmol) and glacial AcOH (1 mL) in MeCN (100 mL) a SelectFluor® (3.50 g; 3.88 mmol) was added at room temperature and then the reaction mixture was stirred at 80° C. for 24 hours. The reaction progress was monitored by LC-MS. When analysis indicated completion of the reaction, the reaction mixture was cooled to room temperature and concentrated in vacuo. The residue was purified by flash column chromatography on silica (column 24 g; hexane 100%, 2 min, hexane/AcOEt, 100:0 to 75:25, v/v, 20 min, 40 mL/min). Compound 45a was obtained in 13% yield (170 mg; 0.69 mmol).

ESI-MS m/z for C₇H₄BrClFN₂ found 249.0/251.0 [M+H]⁻; Rₜ=1.37 min (Method A)

### Step 2.

### Synthesis of tert-butyl (S)-2-((7-bromo-5-chloro-3-fluoro-1H-indazol-1-yl)methyl)morpholine-4-carboxylate (45b)

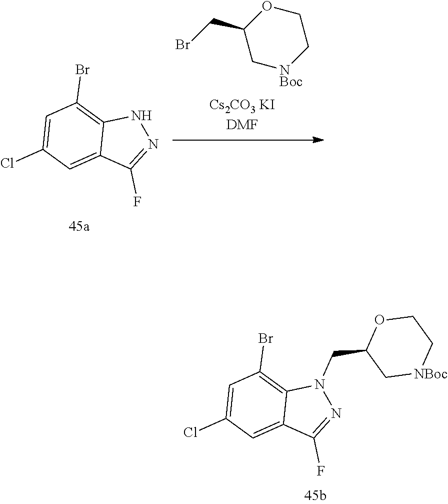 [SMILES: Fc1n[nH]c2c(Br)cc(Cl)cc12 ; CC(C)(C)OC(=O)N1CCO[C@H](CBr)C1 ; CC(C)(C)OC(=O)N1CCO[C@H](Cn2nc(F)c3cc(Cl)cc(Br)c32)C1]

To the solution of 45a (170 mg; 0.690 mmol) and tert-butyl (S)-2-(bromomethyl)morpholine-4-carboxylate (230 mg; 0.820 mmol) in dry DMF (4 mL) Cs₂CO₃ (447 mg; 1.370 mmol) and KI (41 mg; 0.250 mmol) were added and the reaction mixture was stirred under argon atmosphere in a sealed tube at 65° C. overnight. The reaction progress was monitored by LC-MS. When analysis indicated completion of the reaction, the reaction mixture was cooled to room temperature and to this mixture water was added and a whole was extracted with AcOEt (3×). The combined organic layers were dried over anhydrous Na₂SO₄, filtered and concentrated in vacuo. The residue was purified by flash column chromatography on silica (column 12 g; hexane 100%, 2 min, then hexane/AcOEt, 100:0 to 0:100, v/v, 25 min, 23 mL/min). Compound 45b was obtained in 33% yield (100 mg; 0.224 mmol).

ESI-MS m/z for C₁₃H₁₃BrClFN₃O₃ found 392.0/394.0 [M+H-tBu]⁺; Rₜ=1.80 min (Method A)

### Step 3.

### Synthesis of tert-butyl (S)-2-((5-chloro-3-fluoro-7-(4,4,5,5-tetramethyl-1,3,2-dioxaborolan-2-yl)-1H-indazol-1-yl)methyl)morpholine-4-carboxylate (45c)

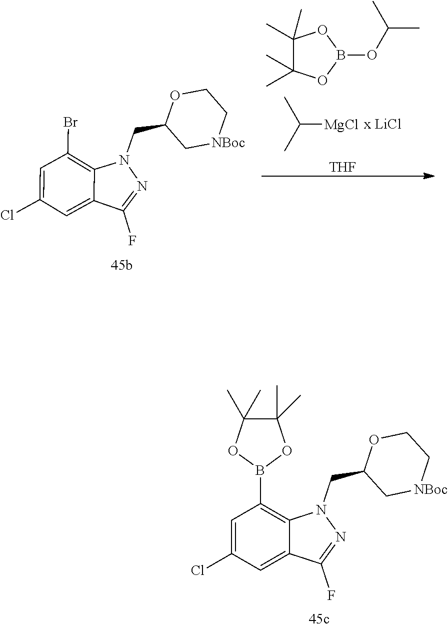

To the solution of isopropyl magnesium chloride lithium chloride complex (1.3 M in THF; 190 μL; 0.246 mmol) in THE (1.6 mL) 45b (100 mg; 0.224 mmol) in THE (1.2 mL) was added at −20° C. and after stirring in this temperature for 30 minutes 2-isopropoxy-4,4,5,5-tetramethyl-1,3,2-dioxaborolane (92 μL; 0.448 mmol) was added. The reaction mixture was allowed to warm to room temperature and stirred for 2 hours. The reaction progress was monitored by LC-MS. When analysis indicated completion of the reaction, the reaction mixture was quenched with water and concentrated in vacuo. The residue was diluted with AcOEt, washed with water and the organic layer was dried over anhydrous Na₂SO₄, filtered and concentrated in vacuo. The crude product was used to the next step without any additional purification.

ESI-MS m/z for C₁₉H₂₅BClFN₃O₅ found 440.0/442.0 [M+H-tBu]⁺; Rₜ=2.22 min (Method A)

### Step 4.

### Synthesis of tert-butyl (2S)-2-((5-chloro-7-(6-((6,6-dimethyl-2,4-dioxo-3-azabicyclo[3.1.0]hexan-3-yl)methyl)pyrrolo[2,1-f][1,2,4]triazin-4-yl)-3-fluoro-1H-indazol-1-yl)methyl)morpholine-4-carboxylate (45d)

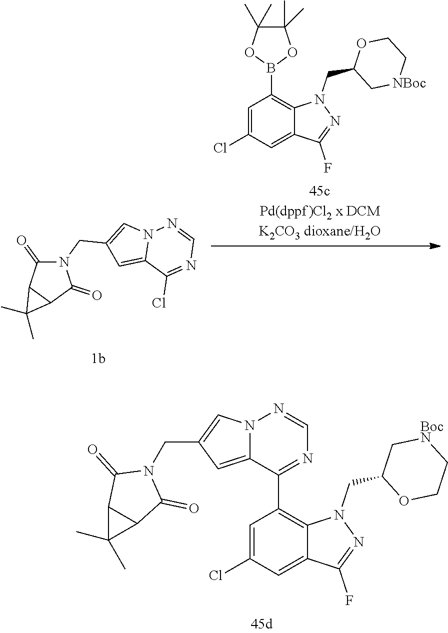 [SMILES: CC(C)(C)OC(=O)N1CCO[C@H](Cn2nc(F)c3cc(Cl)cc(B4OC(C)(C)C(C)(C)O4)c32)C1 ; CC1(C)C2C(=O)N(Cc3cc4c(Cl)ncnn4c3)C(=O)C21 ; CC(C)(C)OC(=O)N1CCO[C@H](Cn2nc(F)c3cc(Cl)cc(-c4ncnn5cc(CN6C(=O)C7C(C6=O)C7(C)C)cc45)c32)C1]

The title compound (45d) was obtained from 1b (57 mg; 0.187 mmol) and from 45c (the crude product) according to the General Procedure IV in 99% yield (per two steps)(118 mg; 0.185 mmol).

ESI-MS m/z for C₃₁H₃₄ClFN₇O₅ found 638.0/640.0 [M+H]⁺; Rₜ=1.86 min (Method A)

### Step 5.

### Synthesis of 3-((4-(5-chloro-3-fluoro-1-(((S)-morpholin-2-yl)methyl)-1H-indazol-7-yl)pyrrolo[2,1-f][1,2,4]triazin-6-yl)methyl)-6,6-dimethyl-3-azabicyclo[3.1.0]hexane-2,4-dione hydrochloride (45)

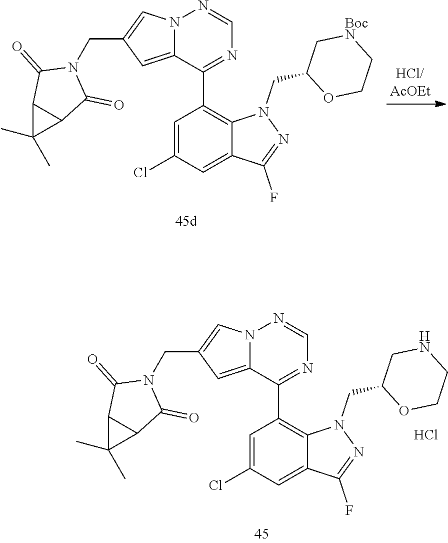 [SMILES: CC(C)(C)OC(=O)N1CCO[C@H](Cn2nc(F)c3cc(Cl)cc(-c4ncnn5cc(CN6C(=O)C7C(C6=O)C7(C)C)cc45)c32)C1 ; CC1(C)C2C(=O)N(Cc3cc4c(-c5cc(Cl)cc6c(F)nn(C[C@@H]7CNCCO7)c56)ncnn4c3)C(=O)C21 ; Cl]

The title compound (45) was obtained as a hydrochloride salt from 45d (118 mg; 0.1f85 mmol) according to the General Procedure IIIa in 71% yield (75 mg; 0.131 mmol). The crude product was purified by preparative reversed-phase column chromatography (C-18, water+0.4‰ TFA/MeCN, 98:2 to 30:70, 30 min, 20 mL/min, Rₜ=19.29 min).

ESI-MS m/z for C₂₆H₂₆ClFN₇O₃ found 538.4/540.4 [M+H]⁺; Rₜ=1.23 min (Method A); ¹H NMR (700 MHz, D₂O) δ 8.58 (s, 1H), 8.14 (s, 1H), 7.94 (s, 1H), 7.71 (d, J=1.7 Hz, 1H), 6.85 (s, 1H), 4.69 (s, 2H), 4.40-4.33 (m, 1H), 4.29-4.22 (m, 1H), 3.75-3.70 (m, 1H), 3.26-3.20 (m, 1H), 3.18-3.12 (m, 2H), 3.01-2.96 (m, 1H), 2.61-2.56 (m, 2H), 2.55-2.49 (m, 1H), 2.45-2.37 (m, 1H), 1.24 (s, 3H), 1.08 (s, 3H).

### Example 46

### Synthesis of 3-((4-(2-((4-oxa-7-azaspiro[2.5]octan-5-yl)methyl)-5-chloro-3-methylphenyl)pyrrolo[2,1-f][1,2,4]triazin-6-yl)methyl)-6,6-dimethyl-3-azabicyclo[3.1.0]hexane-2,4-dione hydrochloride (46)

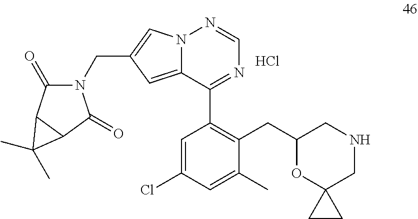 [SMILES: Cc1cc(Cl)cc(-c2ncnn3cc(CN4C(=O)C5C(C4=O)C5(C)C)cc23)c1CC1CNCC2(CC2)O1 ; Cl]

The title compound (46) was obtained as a racemate as a hydrochloride salt in 1% overall yield in a similar way to Example 38 with the exception that, in the first step of the synthesis, 1-(aminomethyl)cyclopropan-1-ol was used instead of 3-amino-2,2-difluoropropan-1-ol and in the last step of the synthesis, the crude product was purified by preparative reversed-phase column chromatography (C-18, water+0.3‰ HCl (36%)/MeCN, 99:1 to 15:85, 30 min, 20 mL/min, Rₜ=16.43 min).

ESI-MS m/z for C₂₈H₃₁ClN₅O₃ found 520.4/522.4 [M+H]⁺; Rₜ=1.26 min (Method A); ¹H NMR (700 MHz, Methanol-d₄) δ 8.61 (s, 1H), 8.27 (d, J=1.4 Hz, 1H), 7.55 (d, J=2.2 Hz, 1H), 7.40 (d, J=2.3 Hz, 1H), 6.87 (d, J=1.4 Hz, 1H), 4.69 (s, 2H), 3.86-3.82 (m, 1H), 3.37-3.34 (m, 1H), 3.29-3.26 (m, 1H), 3.08-3.04 (m, 1H), 3.01-2.98 (m, 1H), 2.85-2.81 (m, 1H), 2.68-2.65 (m, 1H), 2.47 (s, 5H), 1.24 (s, 3H), 1.11 (s, 3H), 0.57-0.52 (m, 2H), 0.40-0.33 (m, 2H).

### Example 47

### Synthesis of 7-(6-((6,6-dimethyl-2,4-dioxo-3-azabicyclo[3.1.0]hexan-3-yl)methyl)pyrrolo[2,1-f][1,2,4]triazin-4-yl)-1-(((S)-morpholin-2-yl)methyl)-1H-indole-5-carboxylic acid hydrochloride (4)

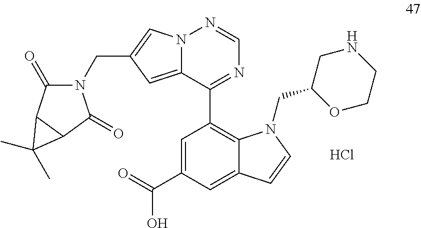 [SMILES: CC1(C)C2C(=O)N(Cc3cc4c(-c5cc(C(=O)O)cc6ccn(C[C@@H]7CNCCO7)c56)ncnn4c3)C(=O)C21 ; Cl]

### Step 1.

### Synthesis of tert-butyl 7-bromo-1H-indole-5-carboxylate (47a)

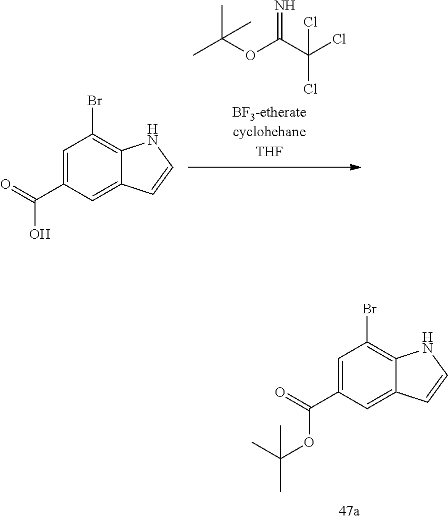 [SMILES: CC(C)(C)OC(=N)C(Cl)(Cl)Cl ; O=C(O)c1cc(Br)c2[nH]ccc2c1 ; CC(C)(C)OC(=O)c1cc(Br)c2[nH]ccc2c1]

To the solution of 7-bromo-1H-indole-5-carboxylic acid (460 mg; 1.92 mmol) in dry THE (3 mL) a solution of tert-butyl 2,2,2-trichloroacetimidate (841 mg; 3.85 mmol) in dry cyclohexane (6 mL) was added dropwise under an Argon atmosphere at room temperature and then to this mixture BF₃-etherate (0.039 mL) was added dropwise and the reaction mixture was stirred at room temperature for 30 minutes. The reaction progress was monitored by LC-MS. When analysis indicated completion of the reaction, the reaction mixture was diluted with 5% NaHCO₃ and extracted with AcOEt. An organic layer was washed with brine, dried over anhydrous Na₂SO₄, filtered and concentrated in vacuo. The residue was purified by flash column chromatography on silica (column 12 g; hexane 100%, 2 min, hexane/AcOEt, 100:0 to 60:40, v/v, 12 min, 23 mL/min). Compound 47a was obtained in 85% yield (481 mg; 1.63 mmol).

ESI-MS m/z for C₁₁H₁₀BrN₂O₂ found 281.0/283.0 [M+H-tBu+MeCN]⁺; Rₜ=1.65 min (Method A)

### Step 2.

### Synthesis of tert-butyl (R)-2-((7-bromo-5-(tert-butoxycarbonyl)-1H-indol-1-yl)methyl)morpholine-4-carboxylate (47b)

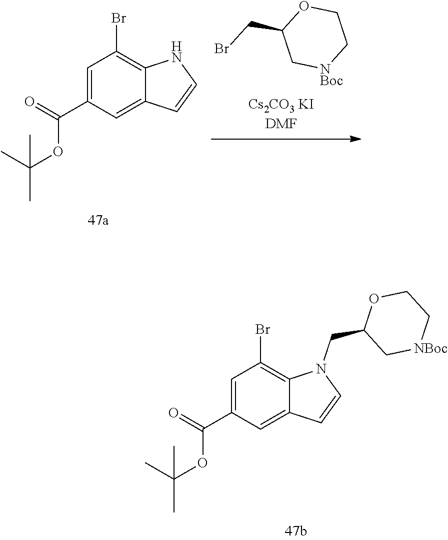 [SMILES: CC(C)(C)OC(=O)c1cc(Br)c2[nH]ccc2c1 ; CC(C)(C)OC(=O)N1CCO[C@H](CBr)C1 ; CC(C)(C)OC(=O)c1cc(Br)c2c(ccn2C[C@@H]2CN(C(=O)OC(C)(C)C)CCO2)c1]

To the solution of 47a (480 mg; 1.627 mmol) and tert-butyl (S)-2-(bromomethyl)morpholine-4-carboxylate (545 mg; 1.952 mmol) in dry DMF (7 mL) Cs₂CO₃ (1.06 g; 3.254 mmol) and KI (97 mg; 0.586 mmol) were added and the reaction mixture was stirred under an Argon atmosphere in a sealed tube at 80° C. overnight. The reaction progress was monitored by LC-MS. When analysis indicated completion of the reaction, the reaction mixture was cooled to room temperature and to this mixture water was added and a whole was extracted with AcOEt (3×). The combined organic layers were dried over anhydrous Na₂SO₄, filtered and concentrated in vacuo. The residue was purified by flash column chromatography on silica (column 24 g; hexane 100%, 2 min, hexane/AcOEt, 100:0 to 50:50, v/v, 18 min, 31 mL/min). Compound 47b was obtained in 75% yield (600 mg; 1.214 mmol).

ESI-MS m/z for C₁₅H₁₆BrN₂O₅ found 383.0/385.0 [M+H-2×tBu]⁺; Rₜ=2.07 min (Method A)

### Step 3.

### Synthesis of tert-butyl (R)-2-((5-(tert-butoxycarbonyl)-7-(4,4,5,5-tetramethyl-1,3,2-dioxaborolan-2-yl)-1H-indol-1-yl)methyl)morpholine-4-carboxylate (47c)

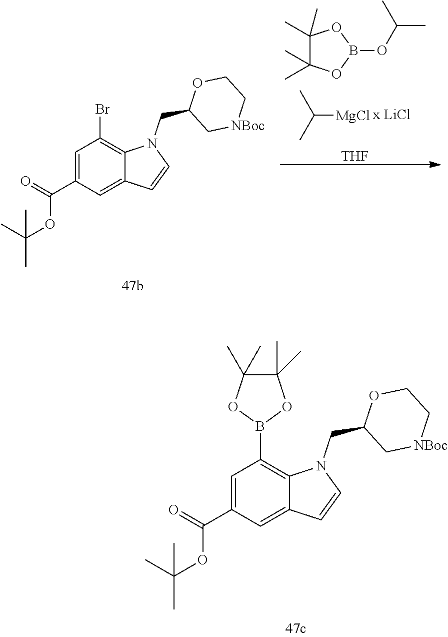

To the solution of 47b (50 mg; 0.101 mmol) in THE (0.8 mL) a solution of isopropyl magnesium chloride lithium chloride complex (1.3 M in THF; 85 μL; 0.111 mmol) in THE (0.8 mL) was slowly added at −10° C. and after stirring in this temperature for 30 minutes 2-isopropoxy-4,4,5,5-tetramethyl-1,3,2-dioxaborolane (41 μL; 0.202 mmol) was added. Then another portions of isopropyl magnesium chloride lithium chloride complex (1.3 M in THF; 3×100 μL; 3×0.130 mmol) was added. When LC-MS indicated no starting material in the reaction mixture 2-isopropoxy-4,4,5,5-tetramethyl-1,3,2-dioxaborolane (2×100 μL; 2×0.493 mmol) was added and the reaction mixture was allowed to warm to room temperature and stirred for 3 hours. The reaction progress was monitored by LC-MS. When analysis indicated completion of the reaction, the reaction mixture was quenched with water and concentrated in vacuo. The residue was then mixed with AcOEt and water and then the layers were separated and an organic one was dried over anhydrous Na₂SO₄, filtered and concentrated in vacuo. The crude product was used to the next step without any additional purification.

ESI-MS m/z for C₂₁H₂₈BN₂O₇ found 431.0/433.0 [M+H-2×tBu]⁺; Rₜ=2.20 min (Method A)

### Step 4.

### Synthesis of tert-butyl (2R)-2-((5-(tert-butoxycarbonyl)-7-(6-((6,6-dimethyl-2,4-dioxo-3-azabicyclo[3.1.0]hexan-3-yl)methyl)pyrrolo[2,1-f][1,2,4]triazin-4-yl)-1H-indol-1-yl)methyl)morpholine-4-carboxylate (47d)

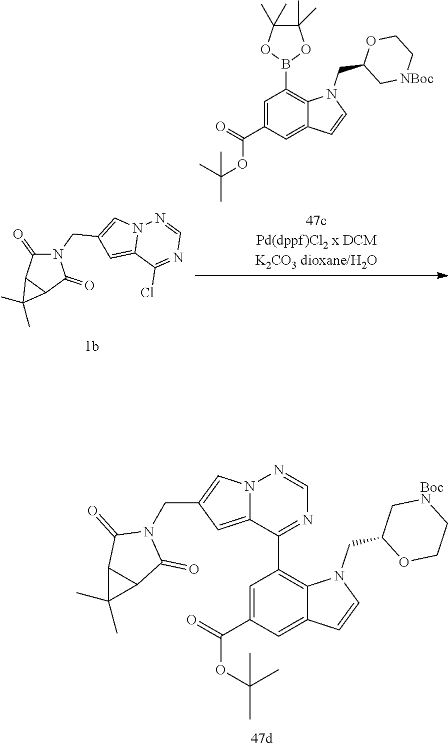 [SMILES: CC(C)(C)OC(=O)c1cc(B2OC(C)(C)C(C)(C)O2)c2c(ccn2C[C@@H]2CN(C(=O)OC(C)(C)C)CCO2)c1 ; CC1(C)C2C(=O)N(Cc3cc4c(Cl)ncnn4c3)C(=O)C21 ; CC(C)(C)OC(=O)c1cc(-c2ncnn3cc(CN4C(=O)C5C(C4=O)C5(C)C)cc23)c2c(ccn2C[C@@H]2CN(C(=O)OC(C)(C)C)CCO2)c1]

The title compound (47d) was obtained from 1b (22 mg; 0.072 mmol) and from 47c (the crude product) according to the General Procedure IV in 57% yield (per two steps)(28 mg; 0.041 mmol).

ESI-MS m/z for C₃₇H₄₅N₆O₇ found 685.0 [M+H]⁺; Rₜ=1.93 min (Method A)

### Step 5.

### Synthesis of 7-(6-((6,6-dimethyl-2,4-dioxo-3-azabicyclo[3.1.0]hexan-3-yl)methyl)pyrrolo[2,1-f][1,2,4]triazin-4-yl)-1-(((S)-morpholin-2-yl)methyl)-1H-indole-5-carboxylic acid hydrochloride (47)

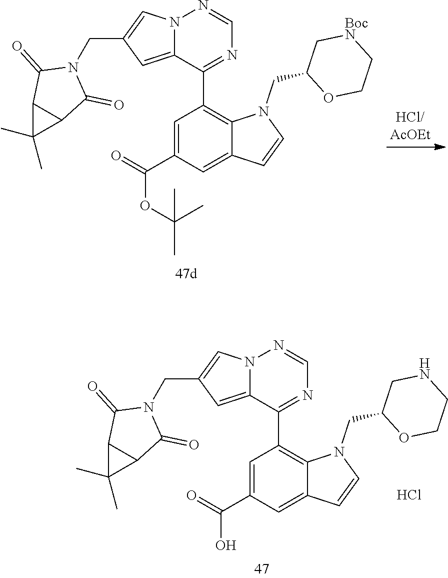 [SMILES: CC(C)(C)OC(=O)c1cc(-c2ncnn3cc(CN4C(=O)C5C(C4=O)C5(C)C)cc23)c2c(ccn2C[C@@H]2CN(C(=O)OC(C)(C)C)CCO2)c1 ; CC1(C)C2C(=O)N(Cc3cc4c(-c5cc(C(=O)O)cc6ccn(C[C@@H]7CNCCO7)c56)ncnn4c3)C(=O)C21 ; Cl]

The title compound (457) was obtained as a hydrochloride salt from 47d (48 mg; 0.070 mmol) according to the General Procedure IIIa in 33% yield (13 mg; 0.023 mmol). The crude product was purified by preparative reversed-phase column chromatography (C-18, water+0.4‰ TFA/MeCN, 98:2 to 30:70, 30 min, 20 mL/min, Rₜ=13.90 min).

ESI-MS m/z for C₂₈H₂₉N₆O₅ found 529.5 [M+H]⁺; Rₜ=0.85 min (Method A); ¹H NMR (700 MHz, D₂O) δ 8.57 (s, 1H), 8.55 (d, J=1.4 Hz, 1H), 8.12 (s, 1H), 7.96 (s, 1H), 7.42 (d, J=3.2 Hz, 1H), 6.93 (d, J=3.3 Hz, 1H), 6.72 (s, 1H), 4.62 (s, 2H), 4.25-4.07 (m, 2H), 3.68-3.60 (m, 1H), 3.43-3.35 (m, 1H), 3.24-3.13 (m, 1H), 3.10-3.00 (m, 2H), 2.71-2.60 (m, 1H), 2.59-2.51 (m, 2H), 2.51-2.42 (m, 1H), 1.23 (s, 3H), 1.05 (s, 3H).

### Example 48

### Synthesis of 3-((4-(5-chloro-3-methyl-2-(piperidin-4-ylmethoxy)phenyl)pyrrolo[2,1-f][1,2,4]triazin-6-yl)methyl)-6,6-dimethyl-3-azabicyclo[3.1.0]hexane-2,4-dione hydrochloride (48)

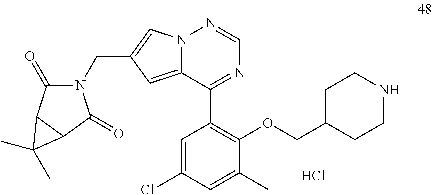 [SMILES: Cc1cc(Cl)cc(-c2ncnn3cc(CN4C(=O)C5C(C4=O)C5(C)C)cc23)c1OCC1CCNCC1 ; Cl]

### Step 1.

### Synthesis of tert-butyl 4-((2-bromo-4-chloro-6-methylphenoxy)methyl)piperidine-1-carboxylate (48a)

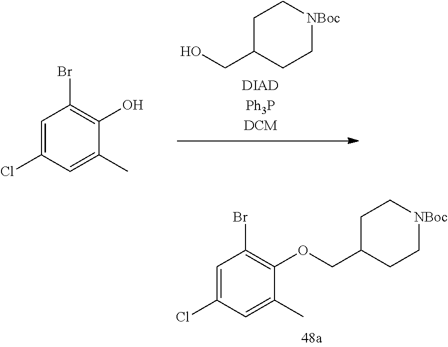

To a solution of 2-bromo-4-chloro-6-methylphenol (370 mg; 1.67 mmol), tert-butyl 4-(hydroxymethyl)piperidine-1-carboxylate (300 mg; 1.39 mmol) and PPh₃ (470 mg; 1.81 mmol) in dry DCM (2.78 mL) DIAD (0.36 mL; 1.81 mmol) was slowly added at room temperature and stirred in this temperature overnight. The solvent was removed in vacuo and the residue was purified by flash column chromatography on silica (hexane/AcOEt, 100:0 to 85:15, v/v, 30 min, 30 mL/min). Compound 48a was obtained in 96% yield (558 mg; 1.34 mmol).

ESI-MS m/z for C₁₈H₂₆BrClNO₃ found 418.0/420.0 [M+H]⁺; Rₜ=2.18 min (Method A); ¹H NMR (700 MHz, CDCl₃) δ 7.37 (dd, J=2.5, 0.7 Hz, 1H), 7.10 (dd, J=2.5, 0.8 Hz, 1H), 4.27-4.08 (m, 2H), 3.73-3.65 (m, 2H), 2.83-2.72 (m, 2H), 2.28 (s, 3H), 2.06-2.00 (m, 1H), 1.91-1.83 (m, 2H), 1.47 (s, 9H), 1.40-1.32 (m, 2H).

### Step 2.

### Synthesis of tert-butyl 4-((4-chloro-2-methyl-6-(4,4,5,5-tetramethyl-1,3,2-dioxaborolan-2-yl)phenoxy)methyl)piperidine-1-carboxylate (48b)

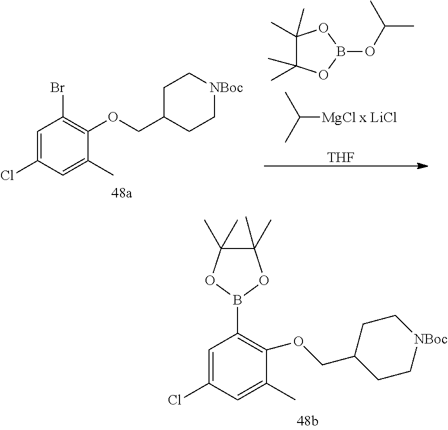

To the solution of 48a (250 mg; 0.597 mmol) in dry THE (1.8 mL) under an Argon atmosphere a isopropyl magnesium chloride lithium chloride complex (1.3 M in THF; 690 μL; 0.895 mmol) was slowly added at −25° C. and stirred for 1.5 hours in a sealed tube. The temperature was kept between −25° C. and −10° C. Then another portion of isopropyl magnesium chloride lithium chloride complex (1.3 M in THF; 460 μL; 0.597 mmol) was added at −25° C. and stirred for 1.5 hours in temperature below −10° C. The mixture was recooled to −30° C. and 2-isopropoxy-4,4,5,5-tetramethyl-1,3,2-dioxaborolane (244 mg; 1.194 mmol) was added. The reaction mixture was allowed to warm to room temperature and stirred for 1.5 hours. The reaction progress was monitored by LC-MS. When analysis indicated completion of the reaction, to the reaction mixture a saturated solution of NH₄Cl (1 mL) was added and a whole was diluted with AcOEt and washed with water. The organic layer was dried over anhydrous MgSO₄, filtered and concentrated in vacuo. The crude product was used to the next step without any additional purification.

ESI-MS m/z for C₂₄H₃₈BClNO₅ found 466.2/468.2 [M+H]⁺; Rₜ=2.31 min (Method A)

### Step 3.

### Synthesis of tert-butyl 4-((4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenoxy)methyl)piperidine-1-carboxylate (48c)

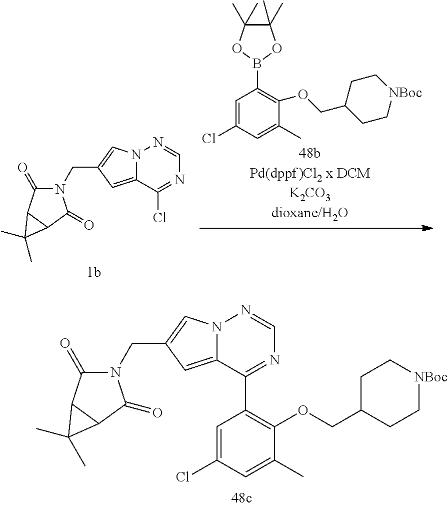 [SMILES: Cc1cc(Cl)cc(B2OC(C)(C)C(C)(C)O2)c1OCC1CCN(C(=O)OC(C)(C)C)CC1 ; CC1(C)C2C(=O)N(Cc3cc4c(Cl)ncnn4c3)C(=O)C21 ; Cc1cc(Cl)cc(-c2ncnn3cc(CN4C(=O)C5C(C4=O)C5(C)C)cc23)c1OCC1CCN(C(=O)OC(C)(C)C)CC1]

The title compound (48c) was obtained from 1b (50 mg; 0.164 mmol) and from 48b (the crude product) according to the General Procedure IV in 26% yield (per two steps)(96 mg; 0.158 mmol).

ESI-MS m/z for C₃₂H₃₉ClN₅O₅ found 608.1/610.1 [M+H]⁺; Rₜ=1.94 min (Method A)

### Step 4.

### Synthesis of 3-((4-(5-chloro-3-methyl-2-(piperidin-4-ylmethoxy)phenyl)pyrrolo[2,1-f][1,2,4]triazin-6-yl)methyl)-6,6-dimethyl-3-azabicyclo[3.1.0]hexane-2,4-dione hydrochloride (48)

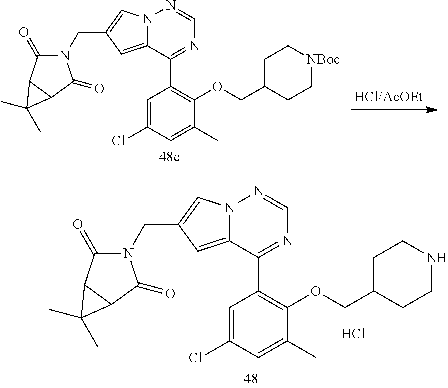 [SMILES: Cc1cc(Cl)cc(-c2ncnn3cc(CN4C(=O)C5C(C4=O)C5(C)C)cc23)c1OCC1CCN(C(=O)OC(C)(C)C)CC1 ; Cc1cc(Cl)cc(-c2ncnn3cc(CN4C(=O)C5C(C4=O)C5(C)C)cc23)c1OCC1CCNCC1 ; Cl]

The title compound (48) was obtained as a hydrochloride salt from 48c (90 mg; 0.148 mmol) according to the General Procedure IIIa in 90% yield (73 mg; 0.134 mmol). The crude product was purified by preparative reversed-phase column chromatography (column: Cosmosil Cholester 20×250 mm, water+0.3‰ HCl (36%)/MeCN, 99:1 to 40:60, 30 min, 20 mL/min).

ESI-MS m/z for C₂₇H₃₁ClN₅O₃ found 508.4/510.4 [M+H]⁺; Rₜ=1.29 min (Method A); ¹H NMR (700 MHz, Methanol-d₄) δ 8.51 (s, 1H), 8.02 (d, J=1.5 Hz, 1H), 7.51-7.48 (m, 1H), 7.41-7.39 (m, 1H), 6.75 (d, J=1.5 Hz, 1H), 4.67 (s, 2H), 3.49-3.45 (m, 2H), 3.29-3.24 (m, 2H), 2.89-2.81 (m, 2H), 2.48 (s, 2H), 2.39 (s, 3H), 1.79-1.72 (m, 1H), 1.61-1.57 (m, 2H), 1.24 (s, 3H), 1.23-1.16 (m, 2H), 1.08 (s, 3H).

### Example 49

### Synthesis of 3-((4-(5-chloro-3-methyl-2-(((5S)-5-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione hydrochloride (49)

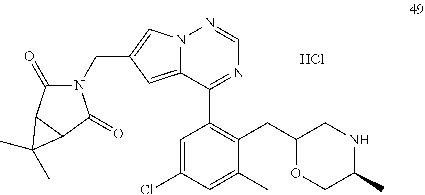

The title compound (49) was obtained as a single diastereoisomer as a hydrochloride salt in 1% overall yield in a similar way to Example 38 with the exception that, in the first step of the synthesis, (S)-2-aminopropan-1-ol was used instead of 3-amino-2,2-difluoropropan-1-ol and in the last step of the synthesis, MeOH was used instead of 6 M HCl. In the fourth step of the synthesis, two diastereoisomers were separated and then the reaction was carried on with one of them. The crude product was purified by preparative reversed-phase column chromatography (column: Cosmosil Cholester 20×250 mm, water+0.3‰ HCl (36%)/MeCN, 99:1 to 20:80, 30 min, 20 mL/min, Rₜ=16.75 min).

ESI-MS m/z for C₂₇H₃₁ClN₅O₃ found 508.4/510.4 [M+H]⁺; Rₜ=1.25 min (Method A); ¹H NMR (700 MHz, Methanol-d₄) δ 8.59 (s, 1H), 8.22 (s, 1H), 7.53 (d, J=2.5 Hz, 1H), 7.38 (d, J=2.3 Hz, 1H), 6.83 (s, 1H), 4.75-4.63 (m, 2H), 3.79-3.64 (m, 2H), 3.26-3.12 (m, 3H), 3.01-2.89 (m, 2H), 2.87-2.77 (m, 1H), 2.49 (s, 3H), 2.47 (s, 2H), 1.23 (s, 3H), 1.14 (d, J=6.4 Hz, 3H), 1.08 (s, 3H).

### Example 50

### Synthesis of 3-((4-(5-chloro-2-((6,6-dimethylmorpholin-2-yl)methyl)-3-methylphenyl)pyrrolo[2,1-f][1,2,4]triazin-6-yl)methyl)-6,6-dimethyl-3-azabicyclo[3.1.0]hexane-2,4-dione hydrochloride (50)

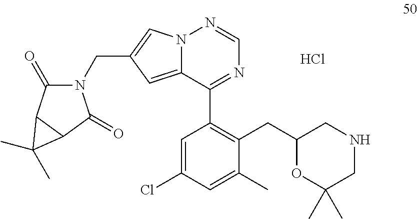 [SMILES: Cc1cc(Cl)cc(-c2ncnn3cc(CN4C(=O)C5C(C4=O)C5(C)C)cc23)c1CC1CNCC(C)(C)O1 ; Cl]

The title compound (50) was obtained as a racemate as a hydrochloride salt in 1% overall yield in a similar way to Example 38 with the exception that, in the first step of the synthesis, 1-amino-2-methylpropan-2-ol was used instead of 3-amino-2,2-difluoropropan-1-ol and the fifth step of the synthesis was different from the one described in Example 38 (the synthesis was as described below). In the last step of the synthesis, MeOH was used instead of 6 M HCl. The crude product was purified by preparative reversed-phase column chromatography (C-18, water+0.3‰ HCl (36%)/MeCN, 99:1 to 20:80, 30 min, 20 mL/min, Rₜ=17.74 min).

ESI-MS m/z for C₂₈H₃₃ClN₅O₃ found 522.5/524.5 [M+H]⁺; Rₜ=1.28 min (Method A); ¹H NMR (700 MHz, Methanol-d₄) δ 8.60 (s, 1H), 8.23 (s, 1H), 7.55 (d, J=2.1 Hz, 1H), 7.41 (d, J=2.1 Hz, 1H), 6.87 (s, 1H), 4.69-4.66 (m, 2H), 3.92-3.87 (m, 1H), 3.19-3.14 (m, 1H), 3.09-3.01 (m, 2H), 2.98-2.92 (m, 1H), 2.69-2.61 (m, 2H), 2.49 (s, 3H), 2.46 (s, 2H), 1.24 (s, 3H), 1.11 (s, 3H), 1.06 (s, 3H), 0.79 (s, 3H).

### Synthesis of 6-(4-chloro-2-methyl-6-(4,4,5,5-tetramethyl-1,3,2-dioxaborolan-2-yl)benzyl)-4-(4-methoxybenzyl)-2,2-dimethylmorpholine (50e)

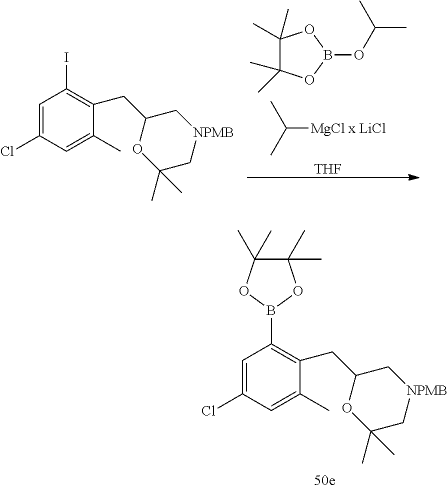

To the solution of 6-(4-chloro-2-iodo-6-methylbenzyl)-4-(4-methoxybenzyl)-2,2-dimethylmorpholine (95 mg; 0.190 mmol) in dry THE (1 mL) isopropyl magnesium chloride lithium chloride complex (1.3 M in THF; 161 μL; 0.210 mmol) was added dropwise at −10° C. and after stirring in this temperature for 1 hour under an Argon atmosphere isopropoxyboronic acid pinacol ester (64 μL; 0.380 mmol) was added dropwise. The reaction mixture was stirred at this temperature for 1 hour. Then the mixture was allowed to warm to room temperature and stirred for 1 hour. The reaction mixture was quenched with an aqueous saturated solution of NH₄Cl and extracted with DCM (3×). The combined organic layers were dried over anhydrous MgSO₄, filtered and concentrated in vacuo. Compound 50e was obtained in 42% yield (40 mg; 0.080 mmol).

ESI-MS m/z for C₂₈H₄₀BClNO₄ found 500.1/502.1 [M+H]⁺; Rₜ=2.02 min (Method A)

### Example 51

### Synthesis of 3-((4-(6-chloro-1-(pyrrolidin-3-yl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione hydrochloride (51)

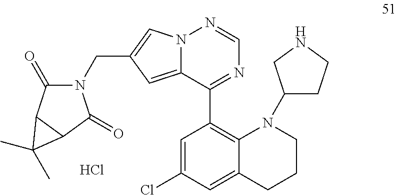 [SMILES: CC1(C)C2C(=O)N(Cc3cc4c(-c5cc(Cl)cc6c5N(C5CCNC5)CCC6)ncnn4c3)C(=O)C21 ; Cl]

### Step 1.

### Synthesis of tert-butyl 3-(6-chloro-3,4-dihydroquinolin-1(2H)-yl)pyrrolidine-1-carboxylate (51a)

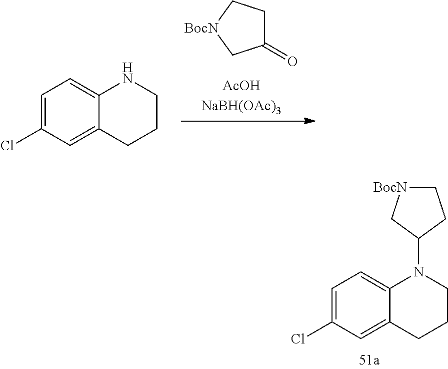 [SMILES: CC(C)(C)OC(=O)N1CCC(=O)C1 ; Clc1ccc2c(c1)CCCN2 ; CC(C)(C)OC(=O)N1CCC(N2CCCc3cc(Cl)ccc32)C1]

The solution of 6-chloro-1,2,3,4-tetrahydroquinoline (500 mg; 2.98 mmol), tert-butyl 3-oxopyrrolidine-1-carboxylate (829 mg; 4.47 mmol), sodium triacetoxyborohydride (NaBH(OAc)₃; 1.58 g; 7.46 mmol) in AcOH (15 mL) was stirred at room temperature overnight. The reaction progress was monitored by LC-MS. When analysis indicated completion of the reaction, to the reaction mixture a saturated solution of NaHCO₃ and 4 M aqueous solution of NaOH were added and the biphasic mixture was stirred for 30 minutes. The layers were separated and the aqueous layer was additionally extracted with DCM (3×). The combined organic extracts were dried over anhydrous Na₂SO₄, filtered and the solvents were evaporated in vacuo. The crude product was purified by flash column chromatography on silica (hexane/AcOEt, 100:0 to 0:100, v/v, 35 min, 30 mL/min). Compound 51a was obtained in 71% yield (715 mg; 2.13 mmol).

ESI-MS m/z for C₁₈H₂₆ClN₂O₂ found 337.2/339.2 [M+H]⁻; Rₜ=4.37 min (Method D)

### Step 2.

### Synthesis of tert-butyl 3-(8-bromo-6-chloro-3,4-dihydroquinolin-1(2H)-yl)pyrrolidine-1-carboxylate (51b)

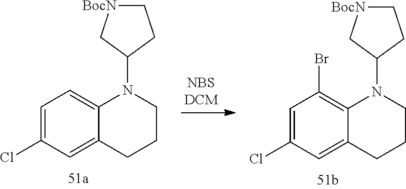 [SMILES: CC(C)(C)OC(=O)N1CCC(N2CCCc3cc(Cl)ccc32)C1 ; CC(C)(C)OC(=O)N1CCC(N2CCCc3cc(Cl)cc(Br)c32)C1]

To the solution of 51a (715 mg; 2.13 mmol) in DCM (21 mL) NBS (416 mg; 2.33 mmol) was added at 0° C. and the resulting mixture was allowed to warm to room temperature over an hour. The reaction progress was monitored by LC-MS. When analysis indicated completion of the reaction, to the reaction mixture water and brine (100 mL) were added and extracted with DCM (3×). The combined organic extracts were dried over anhydrous Na₂SO₄, filtered and concentrated in vacuo. The crude product was purified by flash column chromatography on silica (hexane, 100%, v/v, 15 min). Compound 51b was obtained in 73% yield (645 mg; 1.56 mmol).

ESI-MS m/z for C₁₈H₂₅BrClN₂O₂ found 415.0/417.0 [M+H]⁺; Rₜ=4.77 min (Method D); ¹H NMR (400 MHz, CDCl₃) δ 7.38 (s, 1H), 6.96 (s, 1H), 4.36-4.21 (m, 1H), 3.71-3.62 (m, 1H), 3.59-3.47 (m, 1H), 3.26-3.14 (m, 3H), 3.11-3.00 (m, 1H), 2.79-2.68 (m, 2H), 2.06-1.96 (m, 2H), 1.84-1.75 (m, 2H), 1.46 (s, 9H).

### Step 3.

### Synthesis of (1-(1-(tert-butoxycarbonyl)pyrrolidin-3-yl)-6-chloro-1,2,3,4-tetrahydroquinolin-8-yl)boronic acid (51c)

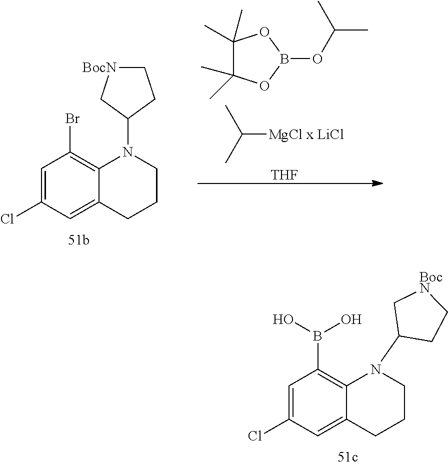

To the cooled to 0° C. solution of isopropyl magnesium chloride lithium chloride complex (1.3 M in THF; 925 μL; 1.203 mmol) in THE (2 mL) a solution of 51b (100 mg; 0.240 mmol) in dry THE (0.6 mL) was slowly added at 0° C. under an Argon atmosphere and the resulting mixture was stirred at 0° C. for 1.5 hours. Then to this mixture 2-isopropoxy-4,4,5,5-tetramethyl-1,3,2-dioxaborolane (134 mg; 0.721 mmol) was added at 0° C. The reaction mixture was allowed to warm to room temperature and stirred for 40 minutes. The reaction progress was monitored by LC-MS. When analysis indicated completion of the reaction, to the reaction mixture water was added and a whole was extracted with AcOEt (3×). The combined organic extracts were dried over anhydrous Na₂SO₄, filtered and concentrated in vacuo. Compound 51c was obtained in 99% yield (90 mg; 0.238 mmol).

ESI-MS m/z for C₁₈H₂₇BClN₂O₄ found 381.2/383.2 [M+H]⁺; Rₜ=3.38 min (Method D)

### Step 4.

### Synthesis of tert-butyl 3-(6-chloro-8-(6-((6,6-dimethyl-2,4-dioxo-3-azabicyclo[3.1.0]hexan-3-yl)methyl)pyrrolo[2,1-f][1,2,4]triazin-4-yl)-3,4-dihydroquinolin-1(2H)-yl)pyrrolidine-1-carboxylate (51d)

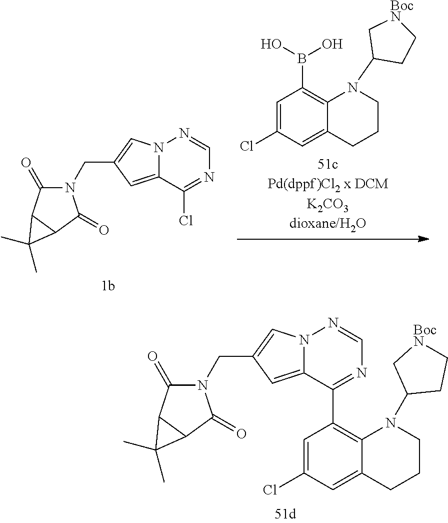 [SMILES: CC1(C)C2C(=O)N(Cc3cc4c(Cl)ncnn4c3)C(=O)C21 ; CC(C)(C)OC(=O)N1CCC(N2CCCc3cc(Cl)cc(B(O)O)c32)C1 ; CC(C)(C)OC(=O)N1CCC(N2CCCc3cc(Cl)cc(-c4ncnn5cc(CN6C(=O)C7C(C6=O)C7(C)C)cc45)c32)C1]

The title compound (51d) was obtained from 1b (73 mg; 0.240 mmol) and from 51c (90 mg; 0.238 mmol) according to the General Procedure IV in 59% yield (85 mg; 0.141 j.w.).

ESI-MS m/z for C₃₂H₃₈ClN₆O₄ found 605.3/607.3 [M+H]⁺; Rₜ=4.23 min (Method D); ¹H NMR (400 MHz, CDCl₃) δ 8.48 (s, 1H), 7.90-7.84 (m, 1H), 7.28-7.26 (m, 1H), 7.14-7.10 (m, 1H), 6.63-6.55 (m, 1H), 4.63-4.57 (m, 2H), 3.65-3.52 (m, 1H), 3.30-3.09 (m, 3H), 2.92-2.61 (m, 4H), 2.61-2.38 (m, 1H), 2.31 (s, 2H), 1.95-1.84 (m, 2H), 1.59-1.51 (m, 2H), 1.38 (s, 9H), 1.21 (s, 3H), 1.04 (s, 3H).

### Step 5.

### Synthesis of 3-((4-(6-chloro-1-(pyrrolidin-3-yl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione hydrochloride (51)

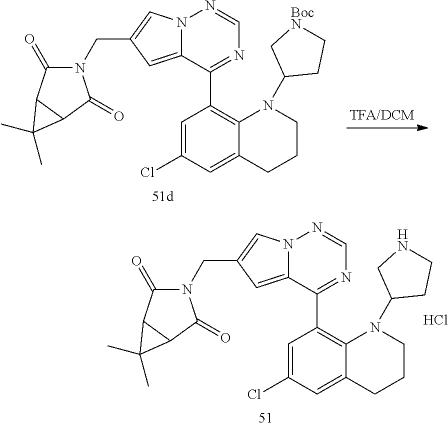 [SMILES: CC(C)(C)OC(=O)N1CCC(N2CCCc3cc(Cl)cc(-c4ncnn5cc(CN6C(=O)C7C(C6=O)C7(C)C)cc45)c32)C1 ; CC1(C)C2C(=O)N(Cc3cc4c(-c5cc(Cl)cc6c5N(C5CCNC5)CCC6)ncnn4c3)C(=O)C21 ; Cl]

The title compound (51) was obtained as a hydrochloride salt as a racemate from 51d (80 mg; 0.132 mmol) according to the General Procedure IIIb in 61% yield (44 mg; 0.081 mmol). The crude product was purified by preparative reversed-phase column chromatography (C-18, water+0.2‰ HCl (36%)/MeCN, 98:2 to 5:95, 30 min, 20 mL/min, Rₜ=15.16 min).

ESI-MS m/z for C₂₇H₃₀ClN₆O₂ found 505.4/507.4 [M+H]⁺; Rₜ=4.81 min (Method D); ¹H NMR (400 MHz, Methanol-d₄) δ 8.67 (s, 1H), 8.36 (d, J=1.5 Hz, 1H), 7.41 (d, J=2.5 Hz, 1H), 7.37-7.34 (m, 1H), 7.16 (d, J=1.5 Hz, 1H), 4.72-4.69 (m, 2H), 3.91-3.79 (m, 1H), 3.36-3.32 (m, 1H), 3.30-3.20 (m, 2H), 3.02-2.83 (m, 4H), 2.51 (s, 2H), 2.50-2.38 (m, 1H), 2.06-1.83 (m, 3H), 1.58-1.43 (m, 1H), 1.26 (s, 3H), 1.13 (s, 3H).

### Example 52

### Synthesis of 3-((4-(5-chloro-3-methyl-2-(((R)-morpholin-2-yl)methoxy)phenyl)pyrrolo[2,1-f][1,2,4]triazin-6-yl)methyl)-6,6-dimethyl-3-azabicyclo[3.1.0]hexane-2,4-dione hydrochloride (52)

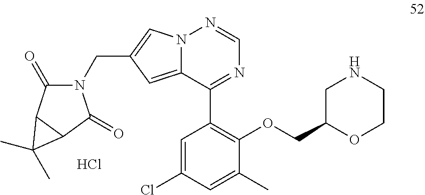 [SMILES: Cc1cc(Cl)cc(-c2ncnn3cc(CN4C(=O)C5C(C4=O)C5(C)C)cc23)c1OC[C@H]1CNCCO1 ; Cl]

The title compound (52) was obtained as a hydrochloride salt in 35% overall yield in a similar way to Example 48 with the exception that, in the first step of the synthesis, tert-butyl (R)-2-(hydroxymethyl)morpholine-4-carboxylate was used instead of tert-butyl 4-(hydroxymethyl)piperidine-1-carboxylate and in the last step of the synthesis, the crude product was purified by preparative reversed-phase column chromatography (C-18, water+0.3‰ HCl (36%)/MeCN, 99:1 to 40:60, 30 min, 20 mL/min, Rₜ=21.25 min).

ESI-MS m/z for C₂₆H₂₉ClN₅O₄ found 510.4/512.4 [M+H]⁺; Rₜ=1.19 min (Method A); ¹H NMR (700 MHz, Methanol-d₄) δ 8.50 (s, 1H), 8.01 (d, J=1.5 Hz, 1H), 7.49 (d, J=2.7 Hz, 1H), 7.38 (d, J=2.6 Hz, 1H), 6.68 (d, J=1.5 Hz, 1H), 4.69 (s, 2H), 3.80-3.74 (m, 2H), 3.70-3.63 (m, 2H), 3.59-3.49 (m, 1H), 3.17-3.09 (m, 1H), 3.04-2.99 (m, 1H), 2.86-2.77 (m, 1H), 2.76-2.69 (m, 1H), 2.51 (s, 2H), 2.39 (s, 3H), 1.24 (s, 3H), 1.05 (s, 3H).

### Example 53

### Synthesis of 3-((4-(5-chloro-3-methyl-2-(((R)-piperidin-3-yl)methoxy)phenyl)pyrrolo[2,1-f][1,2,4]triazin-6-yl)methyl)-6,6-dimethyl-3-azabicyclo[3.1.0]hexane-2,4-dione hydrochloride (53)

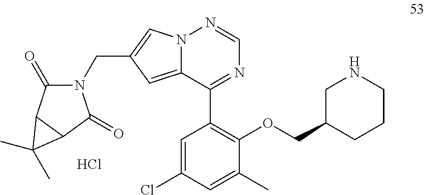 [SMILES: Cc1cc(Cl)cc(-c2ncnn3cc(CN4C(=O)C5C(C4=O)C5(C)C)cc23)c1OC[C@@H]1CCCNC1 ; Cl]

The title compound (53) was obtained as a hydrochloride salt in 25% overall yield in a similar way to Example 48 with the exception that, in the first step of the synthesis, tert-butyl (R)-3-(hydroxymethyl)piperidine-1-carboxylate was used instead of tert-butyl 4-(hydroxymethyl)piperidine-1-carboxylate and in the last step of the synthesis, the crude product was purified by preparative reversed-phase column chromatography (column: Cosmosil Cholester 20×250 mm, water+0.3‰ HCl (36%)/MeCN, 99:1 to 40:60, 30 min, 20 mL/min, Rₜ=19.15 min).

ESI-MS m/z for C₂₇H₃₁ClN₅O₃ found 508.4/510.4 [M+H]⁺; Rₜ=1.24 min (Method A); ¹H NMR (700 MHz, Methanol-d₄) δ 8.51 (s, 1H), 8.01 (d, J=1.5 Hz, 1H), 7.51-7.49 (m, 1H), 7.37 (d, J=2.3 Hz, 1H), 6.68 (d, J=1.5 Hz, 1H), 4.69 (s, 2H), 3.59-3.54 (m, 1H), 3.49-3.45 (m, 1H), 3.29-3.24 (m, 1H), 3.06-3.02 (m, 1H), 2.80-2.74 (m, 1H), 2.51 (s, 2H), 2.48-2.42 (m, 1H), 2.38 (s, 3H), 1.94-1.86 (m, 1H), 1.84-1.78 (m, 1H), 1.65-1.57 (m, 1H), 1.51-1.45 (m, 1H), 1.24 (s, 3H), 1.15-1.08 (m, 1H), 1.06 (s, 3H).

### Example 54

### Synthesis of 3-((4-(6-chloro-1-(piperidin-4-yl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione hydrochloride (54)

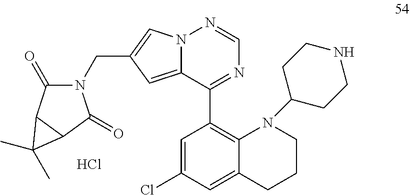 [SMILES: CC1(C)C2C(=O)N(Cc3cc4c(-c5cc(Cl)cc6c5N(C5CCNCC5)CCC6)ncnn4c3)C(=O)C21 ; Cl]

The title compound (54) was obtained as a hydrochloride salt in 2% overall yield in a similar way to Example 51 with the exception that, in the first step of the synthesis, tert-butyl 4-oxopiperidine-1-carboxylate was used instead of tert-butyl 3-oxopyrrolidine-1-carboxylate and in the last step of the synthesis, the crude product was purified by preparative reversed-phase column chromatography (column: Cosmosil Cholester 20×250 mm, water+0.3‰ HCl (36%)/MeCN, 99.9:0.1 to 45:55, 30 min, 15 mL/min, Rₜ=24.40 min).

ESI-MS m/z for C₂₈H₃₂ClN₆O₂ found 519.5/521.5 [M+H]⁺; Rₜ=1.26 min (Method A); ¹H NMR (700 MHz, D₂O) δ 8.51 (s, 1H), 8.01 (d, J=1.5 Hz, 1H), 7.26 (d, J=2.5 Hz, 1H), 7.08 (d, J=2.6 Hz, 1H), 6.61 (d, J=1.5 Hz, 1H), ˜4.8 (2H, overlap with D20 peak), 4.66 (s, 2H), 3.30-3.18 (m, 2H), 3.14-3.07 (m, 2H), 2.92-2.80 (m, 3H), 2.65 (s, 2H), 2.46-2.31 (m, 2H), 1.97-1.87 (m, 2H), 1.76-1.64 (m, 2H), 1.27 (s, 3H), 1.07 (s, 3H).

### Example 55

### Synthesis of 3-((4-(7-chloro-4-(pyrrolidin-3-yl)-3,4-dihydro-2H-benzo[b][1,4]oxazin-5-yl)pyrrolo[2,1-f][1,2,4]triazin-6-yl)methyl)-6,6-dimethyl-3-azabicyclo[3.1.0]hexane-2,4-dione hydrochloride (55)

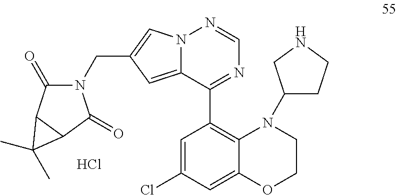 [SMILES: CC1(C)C2C(=O)N(Cc3cc4c(-c5cc(Cl)cc6c5N(C5CCNC5)CCO6)ncnn4c3)C(=O)C21 ; Cl]

The title compound (55) was obtained as a hydrochloride salt in 18% overall yield in a similar way to Example 51 with the exception that, in the first step of the synthesis, 7-chloro-3,4-dihydro-2H-benzo[b][1,4]oxazine was used instead of 6-chloro-1,2,3,4-tetrahydroquinoline and in the last step of the synthesis, the General Procedure IIIa was used instead of the General Procedure IIIb. The crude product was purified by preparative reversed-phase column chromatography (C-18, water+0.2‰ HCl (36%)/MeCN, 98:2 to 5:95, 30 min, 20 mL/min, Rₜ=15.00 min).

ESI-MS m/z for C₂₆H₂₈ClN₆O₃ found 507.2/509.2 [M+H]⁺; Rₜ=4.30 min (Method D); ¹H NMR (400 MHz, Methanol-d₄) δ 8.60 (s, 1H), 8.18 (d, J=1.5 Hz, 1H), 7.10 (s, 2H), 6.91 (d, J=1.5 Hz, 1H), 4.68 (s, 2H), 4.37-4.24 (m, 1H), 4.20-4.08 (m, 1H), 3.80-3.65 (m, 1H), 3.49-3.38 (m, 1H), 3.37-3.31 (m, 1H), 3.29-3.20 (m, 1H), 2.99-2.85 (m, 1H), 2.82-2.68 (m, 1H), 2.50 (s, 2H), 2.40-2.27 (m, 1H), 1.91-1.78 (m, 1H), 1.66-1.49 (m, 1H), 1.25 (s, 3H), 1.11 (s, 3H); ¹³C NMR (101 MHz, Methanol-d₄) δ 175.3, 175.2, 157.6, 150.0, 145.9, 133.7, 128.9, 127.8, 127.1, 124.3, 123.6, 121.8, 121.0, 110.4, 65.6, 65.0, 45.3, 44.8, 41.3, 37.1, 35.3, 34.9, 34.8, 26.2, 26.1, 15.9.

### Example 56

### Synthesis of 3-((4-(5-chloro-3-methyl-2-(1-(piperidin-4-yl)ethoxy)phenyl)pyrrolo[2,1-f][1,2,4]triazin-6-yl)methyl)-6,6-dimethyl-3-azabicyclo[3.1.0]hexane-2,4-dione hydrochloride (56)

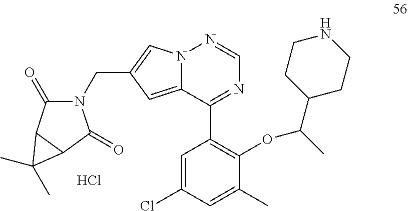 [SMILES: Cc1cc(Cl)cc(-c2ncnn3cc(CN4C(=O)C5C(C4=O)C5(C)C)cc23)c1OC(C)C1CCNCC1 ; Cl]

The title compound (56) was obtained as a single enantiomer as a hydrochloride salt in 26% overall yield in a similar way to Example 48 with the exception that, in the first step of the synthesis, tert-butyl 4-(1-hydroxyethyl)piperidine-1-carboxylate was used instead of tert-butyl 4-(hydroxymethyl)piperidine-1-carboxylate, in the third step of the synthesis, the two enantiomers were separated by chiral preparative HPLC (Lux-Cellulose-4, Flow 15 mL/min; n-hexane:iPA; 90:10 to 10:90, 30 min) and in the last step of the synthesis, the crude product (as a one enantiomer) was purified by preparative reversed-phase column chromatography (column: Cosmosil Cholester 20×250 mm, water+0.3‰ HCl (36%)/MeCN, 99:1 to 40:60, 30 min, 20 mL/min, Rₜ=20.60 min). Analytical control the last compound was carried out by chiral analytical HPLC (Lux-Cellulose-4, Flow 1 mL/min; n-hexane:iPrOH/MeOH (4/1); 60:40 to 10:90, 40 min, Rₜ=30.26 min).

ESI-MS m/z for C₂₈H₃₃ClN₅O₃ found 522.5/524.5 [M+H]⁺; Rₜ=1.32 min (Method A); ¹H NMR (700 MHz, Methanol-d₄) δ 8.53 (s, 1H), 8.06 (d, J=1.4 Hz, 1H), 7.52-7.49 (m, 1H), 7.40 (d, J=2.7 Hz, 1H), 6.81 (d, J=1.4 Hz, 1H), 4.69 (s, 2H), 3.66-3.61 (m, 1H), 3.35-3.32 (m, 1H), 3.30-3.27 (m, 1H), 2.88-2.77 (m, 2H), 2.49 (s, 2H), 2.38 (s, 3H), 1.81-1.75 (m, 1H), 1.65-1.58 (m, 1H), 1.47-1.41 (m, 1H), 1.41-1.35 (m, 1H), 1.26-1.22 (m, 4H), 1.23-1.17 (m, 1H), 1.08 (s, 3H), 0.82 (d, J=6.3 Hz, 3H).

### Example 57

### Synthesis of 3-((4-(5-chloro-3-methyl-2-(1-(piperidin-4-yl)ethoxy)phenyl)pyrrolo[2,1-f][1,2,4]triazin-6-yl)methyl)-6,6-dimethyl-3-azabicyclo[3.1.0]hexane-2,4-dione hydrochloride (57)

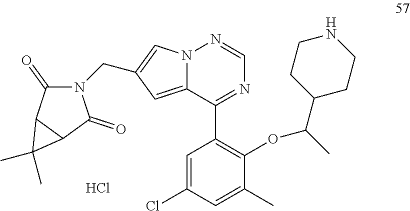 [SMILES: Cc1cc(Cl)cc(-c2ncnn3cc(CN4C(=O)C5C(C4=O)C5(C)C)cc23)c1OC(C)C1CCNCC1 ; Cl]

The title compound (57) was obtained as a second single enantiomer as a hydrochloride salt in 29% overall yield in the same synthesis as for compound 56. In the third step of the synthesis, the two enantiomers were separated by chiral preparative HPLC (Lux-Cellulose-4, Flow 15 mL/min; n-hexane:iPA; 90:10 to 10:90, 30 min) and in the last step of the synthesis, the crude product (as a one enantiomer) was purified by preparative reversed-phase column chromatography (column: Cosmosil Cholester 20×250 mm, water+0.3‰ HCl (36%)/MeCN, 99:1 to 40:60, 30 min, 20 mL/min, Rₜ=20.60 min). Analytical control of the last compound was carried out by chiral analytical HPLC (Lux-Cellulose-4, Flow 1 mL/min; n-hexane:iPrOH/MeOH (4/1); 60:40 to 10:90, 40 min, Rₜ=33.06 min).

ESI-MS m/z for C₂₈H₃₃ClN₅O₃ found 522.5/524.5 [M+H]⁺; Rₜ=1.26 min (Method A); ¹H NMR (700 MHz, Methanol-d₄) δ 8.49 (s, 1H), 8.01-7.98 (m, 1H), 7.50-7.47 (m, 1H), 7.38-7.35 (m, 1H), 6.71-6.67 (m, 1H), 4.68 (s, 2H), 3.63-3.57 (m, 1H), 3.36-3.31 (m, 1H), 3.29-3.23 (m, 1H), 2.87-2.74 (m, 2H), 2.55-2.50 (m, 2H), 2.37 (s, 3H), 1.78-1.71 (m, 1H), 1.63-1.53 (m, 1H), 1.45-1.34 (m, 2H), 1.26-1.18 (m, 4H), 1.05 (s, 3H), 0.82 (d, J=6.3 Hz, 3H).

### Example 58

### Synthesis of 3-((4-(5-chloro-3-methyl-2-((4-methylpiperidin-4-yl)methoxy)phenyl)pyrrolo[2,1-f][1,2,4]triazin-6-yl)methyl)-6,6-dimethyl-3-azabicyclo[3.1.0]hexane-2,4-dione hydrochloride (58)

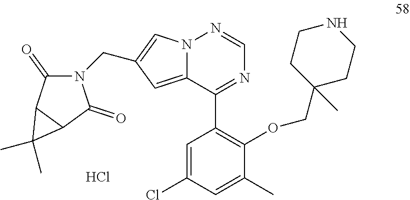 [SMILES: Cc1cc(Cl)cc(-c2ncnn3cc(CN4C(=O)C5C(C4=O)C5(C)C)cc23)c1OCC1(C)CCNCC1 ; Cl]

The title compound (58) was obtained as a hydrochloride salt in 14% overall yield in a similar way to Example 48 with the exception that, in the first step of the synthesis, tert-butyl 4-(hydroxymethyl)-4-methylpiperidine-1-carboxylate was used instead of tert-butyl 4-(hydroxymethyl)piperidine-1-carboxylate and in the last step of the synthesis, the crude product was purified by preparative reversed-phase column chromatography (column: Cosmosil Cholester 20×250 mm, water+0.3‰ HCl (36%)/MeCN, 99:1 to 40:60, 30 min, 20 mL/min, Rₜ=21.10 min).

ESI-MS m/z for C₂₈H₃₃ClN₅O₃ found 522.5/524.5 [M+H]⁺; Rₜ=1.31 min (Method A); ¹H NMR (700 MHz, Methanol-d₄) δ 8.51 (s, 1H), 8.01 (d, J=1.5 Hz, 1H), 7.53-7.48 (m, 1H), 7.39 (d, J=2.6 Hz, 1H), 6.70 (d, J=1.5 Hz, 1H), 4.66 (s, 2H), 3.36 (s, 2H), 3.04-2.93 (m, 4H), 2.47 (s, 2H), 2.40 (s, 3H), 1.50-1.40 (m, 2H), 1.33-1.26 (m, 2H), 1.24 (s, 3H), 1.08 (s, 3H), 0.84 (s, 3H).

### Example 59

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione hydrochloride (59)

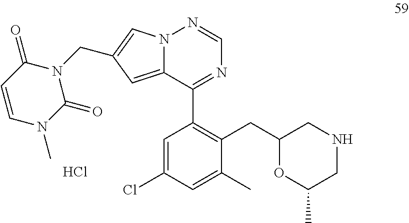 [SMILES: Cc1cc(Cl)cc(-c2ncnn3cc(Cn4c(=O)ccn(C)c4=O)cc23)c1CC1CNC[C@H](C)O1 ; Cl]

### Step 1.

### Synthesis of (6S)-2-(4-chloro-2-methyl-6-(4,4,5,5-tetramethyl-1,3,2-dioxaborolan-2-yl)benzyl)-4-(4-methoxybenzyl)-6-methylmorpholine (59a)

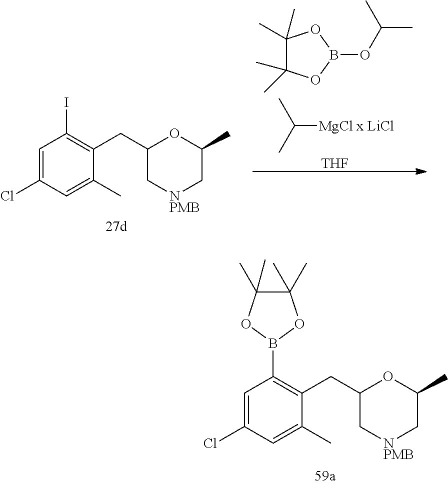

To the cooled to −10° C. solution of 27d (360 mg; 0.74 mmol) in dry THE (2 mL) isopropyl magnesium chloride lithium chloride complex (1.3 M in THF; 1.71 mL; 2.22 mmol) was added dropwise and after stirring in this temperature for 1 hour 2-isopropoxy-4,4,5,5-tetramethyl-1,3,2-dioxaborolane (453 μL; 2.22 mmol) was added dropwise and then the reaction mixture was stirred at 0-10° C. for 1 hour. The reaction mixture was allowed to warm to room temperature and stirred for 1 hour. The reaction progress was monitored by LC-MS. When analysis indicated completion of the reaction, to the reaction mixture a saturated solution of NH₄Cl was added and a whole was extracted with AcOEt (3×). The combined organic layers were dried over anhydrous Na₂SO₄, filtered and concentrated in vacuo. The crude product was used to the next step without any additional purification.

ESI-MS m/z for C₂₇H₃₈BClNO₄ found 486.0/488.0 [M+H]⁺; Rₜ=1.87 min (Method A)

### Step 2.

### Synthesis of 3-((4-chloropyrrolo[2,1-f][1,2,4]triazin-6-yl)methyl)-1-methylpyrimidine-2,4(1H,3H)-dione (59b)

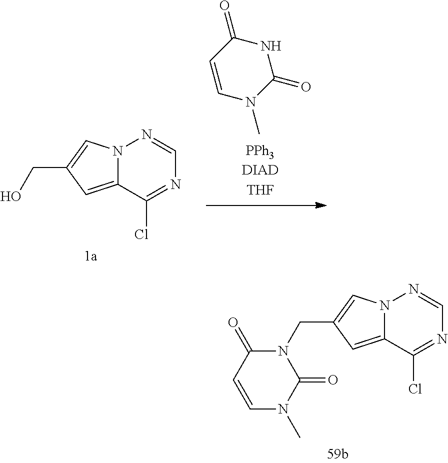

To a cooled to −10° C. solution of 1a (1.248 g; 6.797 mmol), 1-methylpyrimidine-2,4(1H,3H)-dione (787 mg; 6.240 mmol) and PPh₃ (2.236 g; 8.525 mmol) in dry THF (18 mL) DIAD (1.95 mL; 9.904 mmol) was added. The resulting mixture was stirred at room temperature overnight. Then the reaction mixture was concentrated in vacuo and the residue was purified by flash column chromatography on silica (hexane/AcOEt, 100:0 to 0:100, v/v, 35 minutes). Compound 59b was obtained as a white crystals in 15% yield (303 mg; 1.041 mmol).

ESI-MS m/z for C₁₂H₁₁ClN₅O₂ found 292.0/294.0 [M+H]⁺; Rₜ=0.86 min (Method A); ¹H NMR (700 MHz, MeCN-d₃) δ 8.20 (s, 1H), 7.95-7.92 (m, 1H), 7.34 (d, J=7.9 Hz, 1H), 6.96-6.93 (m, 1H), 5.65 (d, J=7.9 Hz, 1H), 5.14 (s, 2H), 3.31 (s, 3H).

### Step 3.

### Synthesis of 3-((4-(5-chloro-2-(((6S)-4-(4-methoxybenzyl)-6-methylmorpholin-2-yl)methyl)-3-methylphenyl)pyrrolo[2,1-f][1,2,4]triazin-6-yl)methyl)-1-methylpyrimidine-2,4(1H,3H)-dione (59c)

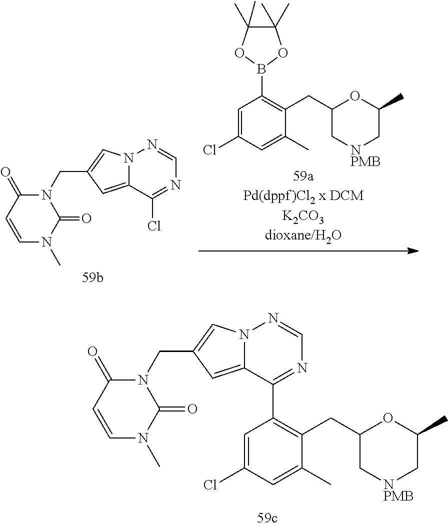

The title compound (59c) was obtained from 59b (60 mg; 0.20 mmol) and from 59a (100 mg; 0.20 mmol) according to the General Procedure IV. The crude product was purified by flash column chromatography on silica (hexane/AcOEt, 100:0 to 85:15, v/v, 30 min, 15 mL/min). Compound 59g was obtained in 95% yield (118 mg; 0.19 mmol).

ESI-MS m/z for C₃₃H₃₆ClN₆O₄ found 615.5/617.5 [M+H]⁺; Rₜ=1.40 min (Method A)

### Step 4.

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione hydrochloride (59)

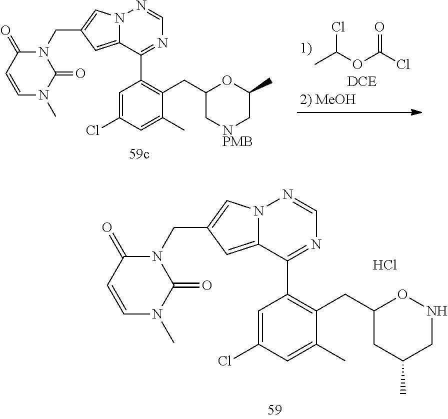

To the cooled to 0° C. solution of 59c (116 mg; 0.189 mmol) in DCE (0.75 mL) 1-chloroethyl carbonochloridate (81 μL; 0.755 mmol) was added dropwise and this mixture was then stirred at 80° C. overnight. The reaction progress was monitored by LC-MS. When the intermediate was formed the solvent was evaporated in vacuo and the residue was dissolved in MeOH (0.98 mL) and whole was stirred at 80° C. for 1 hour. Then the solvent was evaporated in vacuo and to the residue 2 M HCl (a few drops) was added and the crude product was purified by preparative reversed-phase column chromatography (column: Cosmosil Cholester 20×250 mm, water+0.3‰ HCl (36%)/MeCN, 99.9:0.1 to 20:80, 30 min, 20 mL/min, Rₜ=14.00 min). The title compound (59) was obtained as a hydrochloride salt in 97% yield (97 mg; 0.183 mmol).

ESI-MS m/z for C₂₅H₂₈ClN₆O₃ found 495.5/497.5 [M+H]⁺; Rₜ=0.98 min (Method A); ¹H NMR (700 MHz, D₂O) δ 8.38 (s, 1H), 8.00 (d, J=1.5 Hz, 1H), 7.45 (d, J=7.8 Hz, 1H), 7.39-7.36 (m, 1H), 7.18 (d, J=2.3 Hz, 1H), 6.67-6.65 (m, 1H), 5.71 (d, J=7.8 Hz, 1H), 5.04 (s, 2H), 3.64-3.60 (m, 1H), 3.37-3.33 (m, 1H), 3.25 (s, 3H), 3.14-3.07 (m, 2H), 3.00-2.97 (m, 1H), 2.90-2.84 (m, 1H), 2.53-2.49 (m, 1H), 2.36-2.34 (m, 1H), 2.33-2.32 (m, 3H), 0.44 (s, 3H).

### Example 60

### Synthesis of 3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione hydrochloride (60)

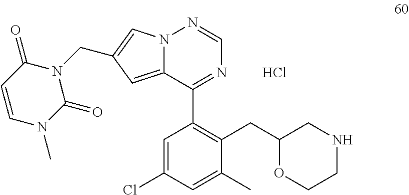 [SMILES: Cc1cc(Cl)cc(-c2ncnn3cc(Cn4c(=O)ccn(C)c4=O)cc23)c1CC1CNCCO1 ; Cl]

### Step 1.

### Synthesis of 4-(4-methoxybenzyl)morpholin-3-one (60a)

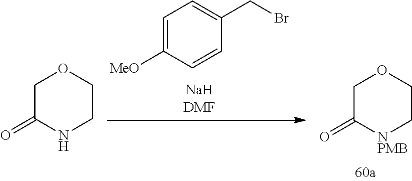

To the cooled to 0° C. solution of morpholin-3-one (500 mg; 4.95 mmol) in dry DMF (10 mL) NaH (60% in mineral oil; 218 mg; 5.44 mmol) was added under an Argon atmosphere and a whole was then stirred for 30 minutes at room temperature. Then to this mixture 1-(bromomethyl)-4-methoxybenzene (0.79 mL; 5.44 mmol) was added dropwise and the reaction mixture was stirred at room temperature for 4 hours. The reaction progress was monitored by LC-MS. When analysis indicated completion of the reaction, the reaction mixture was quenched with 1 M HCl (10 mL) and extracted with AcOEt (2×30 mL). The combined organic layers were washed with water (30 mL) and brine (30 mL), then dried over anhydrous Na₂SO₄, filtered and concentrated in vacuo. The crude product was then purified by flash column chromatography on silica (hexane/AcOEt, 100:0 to 0:100, v/v, 25 min, 15 mL/min). Compound 60a was obtained in 77% yield (847 mg; 3.83 mmol).

ESI-MS m/z for C₁₂H₁₆NO₃ found 222.0 [M+H]⁺; Rₜ=0.80 min (Method A); ¹H NMR (700 MHz, CDCl₃) δ 7.23-7.19 (m, 2H), 6.89-6.86 (m, 2H), 4.56 (s, 2H), 4.23 (s, 2H), 3.84-3.81 (m, 2H), 3.81 (s, 3H), 3.27-3.24 (m, 2H).

### Step 2.

### Synthesis of 2-(4-chloro-2-iodo-6-methylbenzyl)-4-(4-methoxybenzyl)morpholin-3-one (60b)

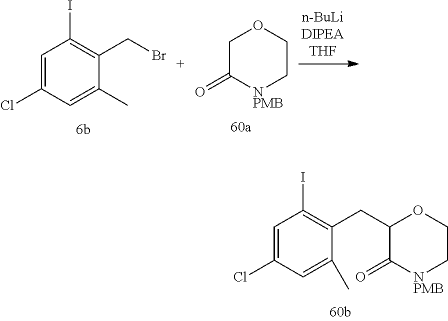

To the cooled to −78° C. solution of DIPEA (279 μL; 1.81 mmol) in dry THE (4 mL) n-BuLi (2.5 M in hexane; 790 μL; 1.99 mmol) was added dropwise and the resulting mixture was warmed to room temperature and stirred at this temperature for 15 minutes. Then the reaction mixture was cooled to −78° C. and to this mixture a solution of 60a (400 mg; 1.81 mmol) in dry THE (2 mL) was added and the mixture was stirred at this temperature for 1 hour. Then to this mixture a solution of 6b (622 mg; 1.81 mmol) in THE (2 mL) was added and a whole was allowed to warm to room temperature overnight. After this time, the reaction mixture was quenched with NH₄Cl and extracted with DCM (3×). The combined organic solutions were dried over anhydrous Na₂SO₄, filtered and concentrated in vacuo. Then the crude product was purified by flash column chromatography on silica (hexane/AcOEt, 100:0 to 0:100, v/v, 25 min, 15 mL/min). Compound 60b was obtained in 66% yield (585 mg; 1.20 mmol).

ESI-MS m/z for C₂₀H₂₂ClINO₃ found 486.0/488.0 [M+H]⁺; Rₜ=1.85 min (Method A)

### Step 3.

### Synthesis of 2-(4-chloro-2-iodo-6-methylbenzyl)-4-(4-methoxybenzyl)morpholine (60c)

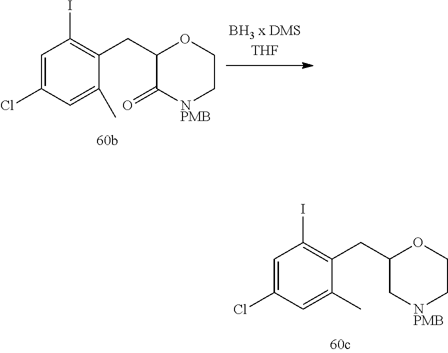

To the cooled to 0° C. solution of 60b (585 mg; 1.20 mmol) in dry THE (10 mL) BH₃×DMS (228 μL; 2.41 mmol) was slowly added under an Argon atmosphere and this mixture was then stirred at 65° C. for 2 hours. Then to the reaction mixture 6 M HCl (3 mL) was added and stirred at 65° C. for 30 minutes. Then the reaction mixture was cooled to 0° C. and a whole was alkalized with 4 M NaOH and then extracted with AcOEt (2×20 mL). The combined organic layers were washed with brine (20 mL) and dried over anhydrous MgSO₄, filtered and concentrated in vacuo. The crude product was purified by flash column chromatography on silica (hexane/AcOEt, 100:0 to 0:100, v/v, 25 min, 20 mL/min). Compound 60c was obtained in 88% yield (500 mg; 1.06 mmol).

ESI-MS m/z for C₂₀H₂₄ClINO₂ found 471.9/473.8 [M+H]⁺; Rₜ=1.64 min (Method A); ¹H NMR (700 MHz, CDCl₃) δ 7.68 (d, J=2.2 Hz, 1H), 7.24-7.20 (m, 2H), 7.12 (d, J=2.2 Hz, 1H), 6.87-6.84 (m, 2H), 3.80 (s, 3H), 3.80-3.76 (m, 2H), 3.57-3.50 (m, 2H), 3.39 (d, J=12.9 Hz, 1H), 3.03-2.97 (m, 1H), 2.88-2.79 (m, 2H), 2.62-2.57 (m, 1H), 2.39 (s, 3H), 2.14-2.04 (m, 2H).

### Step 4.

### Synthesis of 2-(4-chloro-2-methyl-6-(4,4,5,5-tetramethyl-1,3,2-dioxaborolan-2-yl)benzyl)-4-(4-methoxybenzyl)morpholine (60d)

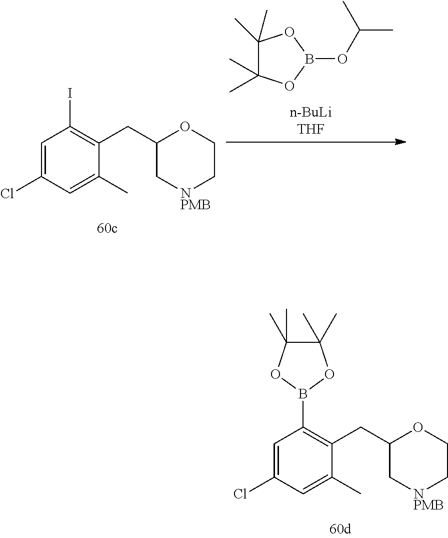

To the cooled to −78° C. solution of 60c (75 mg; 0.159 mmol) in dry THE (1.5 mL) n-BuLi (2.5 M in hexane; 76 μL; 0.191 mmol) was slowly added and after stirring in this temperature for 15 minutes 2-isopropoxy-4,4,5,5-tetramethyl-1,3,2-dioxaborolane (49 μL; 0.238 mmol) was added dropwise and then the reaction mixture was allowed to warm to room temperature and stirred for 1 hour. The reaction progress was monitored by LC-MS. When analysis indicated completion of the reaction, to the reaction mixture a saturated solution of NH₄Cl (10 mL) was added and a whole was extracted with AcOEt (2×10 mL). The combined organic layers were washed with brine, dried over anhydrous Na₂SO₄, filtered and concentrated in vacuo. Compound 60d was obtained in 96% yield (72 mg; 0.153 mmol).

ESI-MS m/z for C₂₆H₃₆BClNO₄ found 472.30/474.2 [M+H]⁺; Rₜ=1.69 min (Method A)

### Step 5.

### Synthesis of 3-((4-(5-chloro-2-((4-(4-methoxybenzyl)morpholin-2-yl)methyl)-3-methylphenyl)pyrrolo[2,1-f][1,2,4]triazin-6-yl)methyl)-1-methylpyrimidine-2,4(1H,3H)-dione (60e)

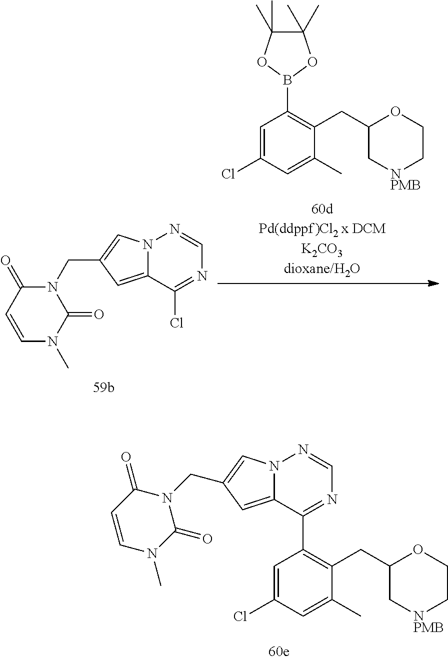

The title compound (60e) was obtained from 59b (43 mg; 0.148 mmol) and from 60d (70 mg; 0.148 mmol) according to the General Procedure IV. The crude product was purified by flash column chromatography on silica (hexane/AcOEt, 100:0 to 0:100, v/v, 20 min, 13 mL/min). Compound 60e was obtained in 19% yield (17 mg; 0.028 mmol).

ESI-MS m/z for C₃₂H₃₄ClN₆O₄ found 601.2/603.2 [M+H]⁺; Rₜ=1.32 min (Method A)

### Step 6.

### Synthesis of 3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione hydrochloride (60)

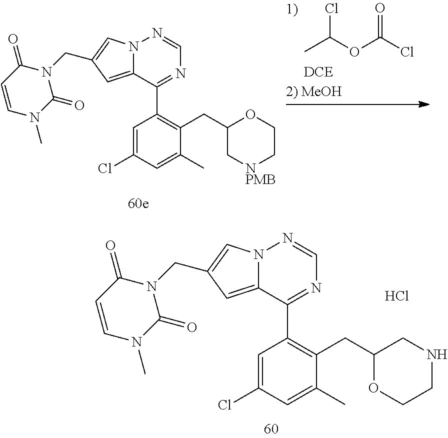

To the cooled to 0° C. solution of 60e (16 mg; 0.027 mmol) in DCE (0.1 mL) 1-chloroethyl carbonochloridate (12 μL; 0.106 mmol) was added dropwise and this mixture was then stirred at 80° C. overnight. The reaction progress was monitored by LC-MS. When analysis indicated completion of the reaction, the solvent was evaporated in vacuo and the residue was dissolved in MeOH (0.5 mL) and whole was refluxed for 30 minutes. Then the solvent was evaporated in vacuo and the crude product was purified by preparative reversed-phase column chromatography (column: Cosmosil Cholester 20×250 mm, water+0.3‰ HCl (36%)/MeCN, 98:2 to 30:70, 30 min, 20 mL/min, Rₜ=12.50 min). The title compound (60) was obtained as a racemate as a hydrochloride salt in 37% yield (5 mg; 0.010 mmol).

ESI-MS m/z for C₂₄H₂₆ClN₆O₃ found 481.4/483.4 [M+H]⁺; Rₜ=1.01 min (Method A); ¹H NMR (700 MHz, D₂O) δ 8.61-8.55 (m, 1H), 8.33 (t, J=2.5 Hz, 1H), 7.61 (d, J=7.9 Hz, 1H), 7.59-7.55 (m, 1H), 7.31 (d, J=2.3 Hz, 1H), 6.97 (s, 1H), 5.87 (d, J=7.8 Hz, 1H), 5.24 (s, 2H), 3.73-3.66 (m, 2H), 3.48-3.42 (m, 1H), 3.38 (s, 3H), 3.26-3.20 (m, 1H), 3.19-3.12 (m, 1H), 3.03-2.98 (m, 1H), 2.96-2.88 (m, 2H), 2.78-2.71 (m, 1H), 2.45 (s, 3H).

### Example 61

### Synthesis of 3-((4-(5-chloro-1-((4-fluoropiperidin-4-yl)methyl)-1H-indol-7-yl)pyrrolo[2,1-f][1,2,4]triazin-6-yl)methyl)-1-methylpyrimidine-2,4(1H,3H)-dione hydrochloride (61)

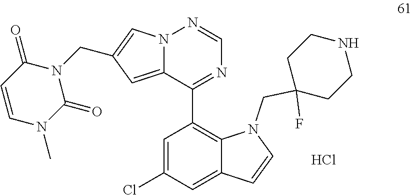 [SMILES: Cn1ccc(=O)n(Cc2cc3c(-c4cc(Cl)cc5ccn(CC6(F)CCNCC6)c45)ncnn3c2)c1=O ; Cl]

The title compound (61) was obtained as a hydrochloride salt in 2% overall yield in a similar way to Example 10 with the exception that, in the fourth step of the synthesis, the compound 59b was used instead of the compound 1b and in the last step of the synthesis, the crude product was purified by preparative reversed-phase column chromatography (column: Cosmosil Cholester 20×250 mm, water+0.3‰ HCl (36%)/MeCN, 99:1 to 20:80, 30 min, 20 mL/min, Rₜ=14.00 min).

ESI-MS m/z for C₂₆H₂₆ClFN₇O₂ found 522.4/524.4 [M+H]⁺; Rₜ=1.10 min (Method A); ¹H NMR (700 MHz, D₂O) δ 8.61 (s, 1H), 8.27 (d, J=1.4 Hz, 1H), 7.97-7.89 (m, 1H), 7.57 (d, J=7.8 Hz, 1H), 7.45-7.38 (m, 2H), 6.94 (d, J=1.5 Hz, 1H), 6.79 (d, J=3.2 Hz, 1H), 5.84 (d, J=7.8 Hz, 1H), 5.22 (s, 2H), 4.77-4.74 (m, 2H), 4.32-4.14 (m, 2H), 3.34 (s, 3H), 3.21-3.14 (m, 2H), 2.86-2.78 (m, 2H), 1.69-1.56 (m, 2H), 1.42-1.34 (m, 2H).

### Example 62

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1-(2,2,2-trifluoroethyl)pyrimidine-2,4(1H,3H)-dione hydrochloride (62)

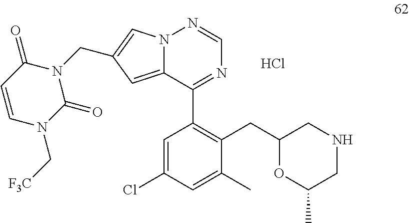 [SMILES: Cc1cc(Cl)cc(-c2ncnn3cc(Cn4c(=O)ccn(CC(F)(F)F)c4=O)cc23)c1CC1CNC[C@H](C)O1 ; Cl]

The title compound (62) was obtained as a hydrochloride salt in 6% overall yield in a similar way to Example 59 with the exception that, in the second step of the synthesis, 1-(2,2,2-trifluoroethyl)pyrimidine-2,4(1H,3H)-dione was used instead of 1-methylpyrimidine-2,4(1H,3H)-dione and in the last step of the synthesis, the crude product was purified by preparative reversed-phase column chromatography (C-18, water+0.3‰ HCl (36%)/MeCN, 99.9:0.1 to 20:80, 30 min, 20 mL/min, Rₜ=16.20 min).

ESI-MS m/z for C₂₆H₂₆ClF₃N₆O₃ found 563.2/565.2 [M+H]⁺; Rₜ=1.12 min (Method A); ¹H NMR (700 MHz, D₂O) δ 8.39 (s, 1H), 8.04 (s, 1H), 7.56 (d, J=8.0 Hz, 1H), 7.40 (d, J=2.0 Hz, 1H), 7.22 (d, J=2.0 Hz, 1H), 6.69 (s, 1H), 5.83 (d, J=8.0 Hz, 1H), 5.08 (s, 2H), 4.53-4.49 (m, 2H), 3.64-3.60 (m, 1H), 3.35-3.30 (m, 1H), 3.15-3.07 (m, 2H), 2.99-2.96 (m, 1H), 2.93-2.86 (m, 1H), 2.53-2.47 (m, 1H), 2.32 (s, 4H), 0.40 (s, 3H).

### Example 63

### Synthesis of 3-((4-(6-chloro-1-((4-fluoropiperidin-4-yl)methyl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione hydrochloride (63)

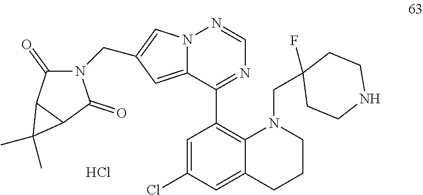 [SMILES: CC1(C)C2C(=O)N(Cc3cc4c(-c5cc(Cl)cc6c5N(CC5(F)CCNCC5)CCC6)ncnn4c3)C(=O)C21 ; Cl]

### Step 1.

### Synthesis of tert-butyl 4-((6-chloro-3,4-dihydroquinolin-1(2H)-yl)methyl)-4-hydroxypiperidine-1-carboxylate (63a)

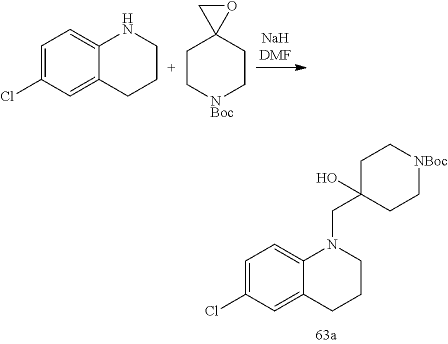

To the cooled to 0° C. solution of 6-chloro-1,2,3,4-tetrahydroquinoline (100 mg; 0.59 mmol) in dry DMF (6 mL) NaH (60% in mineral oil; 65 mg; 2.68 mmol) was added and the resulting mixture was then stirred for 10 minutes. Then to this solution tert-butyl 1-oxa-6-azaspiro[2.5]octane-6-carboxylate (191 mg; 0.89 mmol) was added and the reaction mixture was heated at 70° C. for 1.5 hours and then at room temperature overnight. The reaction progress was monitored by LC-MS. When analysis indicated completion of the reaction, a whole was concentrated in vacuo. The crude product was then purified by flash column chromatography on silica (hexane/AcOEt, 100:0 to 0:100, v/v, 30 min, 40 mL/min). Compound 63a was obtained in 83% yield (186 mg; 0.49 mmol).

ESI-MS m/z for C₂₀H₃₀ClN₂O₃ found 381.0/383.0 [M+H]⁺; Rₜ=1.83 min (Method C)

### Step 2.

### Synthesis of tert-butyl 4-((6-chloro-3,4-dihydroquinolin-1(2H)-yl)methyl)-4-fluoropiperidine-1-carboxylate (63b)

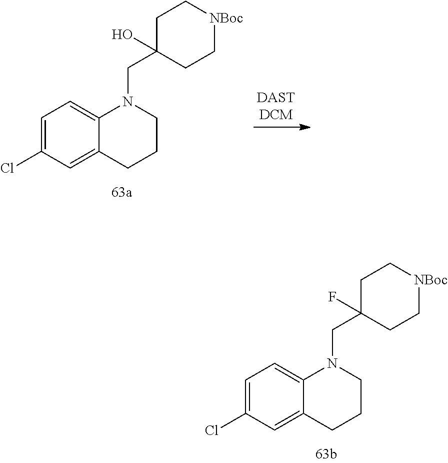 [SMILES: CC(C)(C)OC(=O)N1CCC(O)(CN2CCCc3cc(Cl)ccc32)CC1 ; CC(C)(C)OC(=O)N1CCC(F)(CN2CCCc3cc(Cl)ccc32)CC1]

To a cooled to −20° C. solution of 63a (186 mg; 0.49 mmol) in dry DCM (2.2 mL) DAST (0.1 mL; 0.73 mmol) was added dropwise. Then, this mixture was stirred at −20° C. for 1 hour. The reaction progress was monitored by LC-MS. When analysis indicated completion of the reaction, the reaction was quenched with 5% NaHCO₃. The phases were separated and an aqueous one was extracted with DCM (3×). The combined organic solutions were washed with brine and then dried over anhydrous Na₂SO₄, filtered and concentrated in vacuo. Compound 63b was obtained in 99% yield (183 mg; 0.48 mmol).

ESI-MS m/z for C₂₀H₂₉ClFN₂O₂ found 383.1/385.1 [M+H]⁺; Rₜ=2.07 min (Method C); ¹H NMR (400 MHz, CDCl₃) δ 6.99-6.93 (m, 1H), 6.92-6.89 (m, 1H), 6.55-6.51 (m, 1H), 4.08-3.91 (m, 2H), 3.40-3.31 (m, 3H), 3.08-2.98 (m, 2H), 2.79-2.71 (m, 2H), 1.95-1.84 (m, 4H), 1.68-1.51 (m, 3H), 1.46 (s, 9H).

### Step 3.

### Synthesis of tert-butyl 4-((8-bromo-6-chloro-3,4-dihydroquinolin-1(2H)-yl)methyl)-4-fluoropiperidine-1-carboxylate (63c)

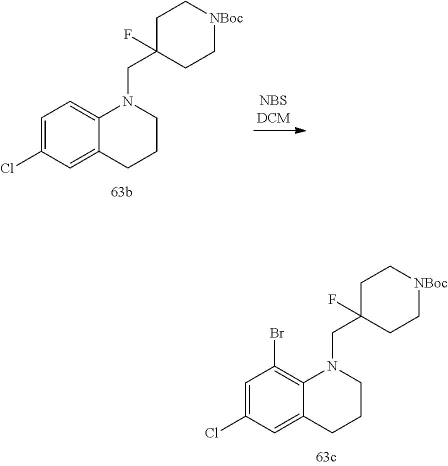 [SMILES: CC(C)(C)OC(=O)N1CCC(F)(CN2CCCc3cc(Cl)ccc32)CC1 ; CC(C)(C)OC(=O)N1CCC(F)(CN2CCCc3cc(Cl)cc(Br)c32)CC1]

To the solution of 63b (183 mg; 0.48 mmol) in DCM (5 mL) NBS (96 mg; 0.54 mmol) was added at 0° C. and the resulting mixture was allowed to warm to room temperature over an hour. The reaction progress was monitored by LC-MS. When analysis indicated completion of the reaction, to the reaction mixture brine was added and then a whole was extracted with DCM (3×). The combined organic extracts were dried over anhydrous Na₂SO₄, filtered and concentrated in vacuo. Compound 63c was obtained in 94% yield (208 mg; 0.45 mmol).

ESI-MS m/z for C₂₀H₂₈BrClFN₂O₂ found 461.0/462.9 [M+H]⁺; Rₜ=2.27 min (Method C); ¹H NMR (400 MHz, CDCl₃) δ 7.36-7.34 (m, 1H), 7.01-6.98 (m, 1H), 4.11-3.95 (m, 2H), 3.28-3.18 (m, 4H), 3.11-2.98 (m, 2H), 2.81-2.74 (m, 3H), 2.04-1.93 (m, 2H), 1.87-1.78 (m, 3H), 1.51-1.44 (m, 9H).

### Step 4.

### Synthesis of (1-((1-(tert-butoxycarbonyl)-4-fluoropiperidin-4-yl)methyl)-6-chloro-1,2,3,4-tetrahydroquinolin-8-yl)boronic acid (63d)

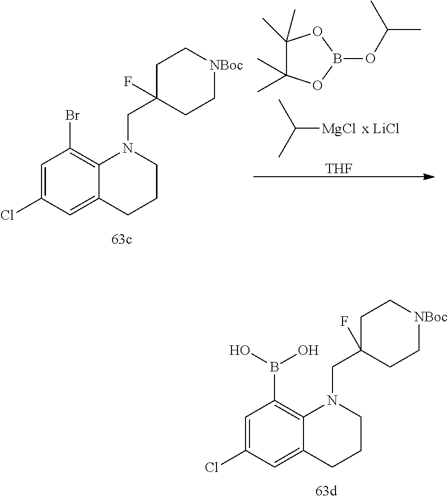

To the cooled to 0° C. solution of isopropyl magnesium chloride lithium chloride complex (1.3 M in THF; 1.65 mL; 2.165 mmol) in THF (2.6 mL) a solution of 63c (200 mg; 0.433 mmol) in dry THE (2 mL) was slowly added at 0° C. under an Argon atmosphere and the resulting mixture was stirred at 0° C. for 1.5 hours. Then to this mixture 2-isopropoxy-4,4,5,5-tetramethyl-1,3,2-dioxaborolane (242 mg; 1.300 mmol) was added at 0° C. The reaction mixture was allowed to warm to room temperature and stirred for 40 minutes. The reaction progress was monitored by LC-MS. When analysis indicated completion of the reaction, to the reaction mixture 2 M HCl and water were added (to pH about 4) and a whole was extracted with AcOEt (3×). The combined organic extracts were washed with brine and dried over anhydrous Na₂SO₄, filtered and concentrated in vacuo. The crude product was used to the next step without any additional purification.

ESI-MS m/z for C₂₀H₃₀BClFN₂O₄ found 427.1/429.1 [M+H]⁺; Rₜ=1.73 min (Method C)

### Step 5.

### Synthesis of tert-butyl 4-((6-chloro-8-(6-((6,6-dimethyl-2,4-dioxo-3-azabicyclo[3.1.0]hexan-3-yl)methyl)pyrrolo[2,1-f][1,2,4]triazin-4-yl)-3,4-dihydroquinolin-1(2H)-yl)methyl)-4-fluoropiperidine-1-carboxylate (63e)

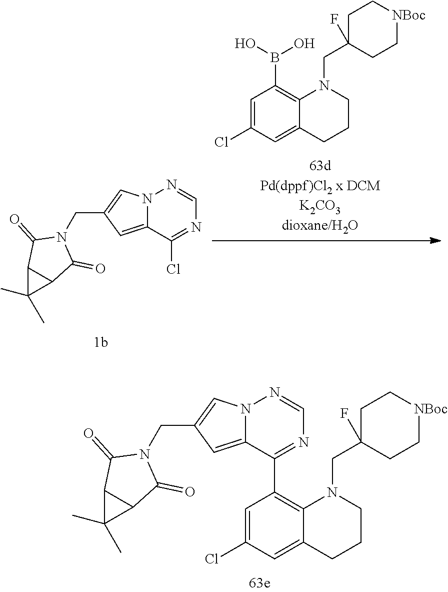 [SMILES: CC(C)(C)OC(=O)N1CCC(F)(CN2CCCc3cc(Cl)cc(B(O)O)c32)CC1 ; CC1(C)C2C(=O)N(Cc3cc4c(Cl)ncnn4c3)C(=O)C21 ; CC(C)(C)OC(=O)N1CCC(F)(CN2CCCc3cc(Cl)cc(-c4ncnn5cc(CN6C(=O)C7C(C6=O)C7(C)C)cc45)c32)CC1]

The title compound (63e) was obtained from 1b (132 mg; 0.433 mmol) and from 63d (the crude product) according to the General Procedure IV in 62% yield (per two steps)(174 mg; 0.268 mmol).

ESI-MS m/z for C₃₄H₄₁ClFN₆O₄ found 651.3/653.3 [M+H]⁺; Rₜ=2.04 min (Method C)

### Step 6.

### Synthesis of 3-((4-(6-chloro-1-((4-fluoropiperidin-4-yl)methyl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione hydrochloride (63)

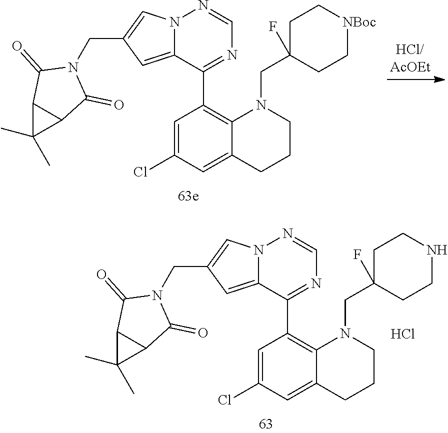 [SMILES: CC(C)(C)OC(=O)N1CCC(F)(CN2CCCc3cc(Cl)cc(-c4ncnn5cc(CN6C(=O)C7C(C6=O)C7(C)C)cc45)c32)CC1 ; CC1(C)C2C(=O)N(Cc3cc4c(-c5cc(Cl)cc6c5N(CC5(F)CCNCC5)CCC6)ncnn4c3)C(=O)C21 ; Cl]

The title compound (63) was obtained as a hydrochloride salt as a racemate from 63e (174 mg; 0.268 mmol) according to the General Procedure IIIa in 53% yield (83 mg; 0.141 mmol). The crude product was purified by preparative reversed-phase column chromatography (C-18, water+1‰ HCl (36%)/MeCN, 98:2 to 5:95, 30 min, 20 mL/min, Rₜ=15.41 min).

ESI-MS m/z for C₂₉H₃₃ClFN₆O₂ found 551.2/553.2 [M+H]⁺; Rₜ=2.65 min (Method D); ¹H NMR (400 MHz, Methanol-d₄) δ 8.64 (s, 1H), 8.31 (d, J=1.5 Hz, 1H), 7.38-7.32 (m, 2H), 7.11 (d, J=1.5 Hz, 1H), 4.71 (s, 2H), 3.38-3.32 (m, 2H), 3.21-3.13 (m, 2H), 3.01-2.87 (m, 6H), 2.50 (s, 2H), 2.06-1.96 (m, 2H), 1.73-1.64 (m, 2H), 1.56-1.34 (m, 2H), 1.25 (s, 3H), 1.10 (s, 3H); ¹⁹F NMR (376 MHz, Methanol-d₄) δ −161.48.

### Example 64

### Synthesis of 3-((4-(6-chloro-1-(pyrrolidin-3-yl)-1,2,3,4-tetrahydroquinolin-8-yl)pyrrolo[2,1-f][1,2,4]triazin-6-yl)methyl)-1-(2,2,2-trifluoroethyl)pyrimidine-2,4(1H,3H)-dione hydrochloride (64)

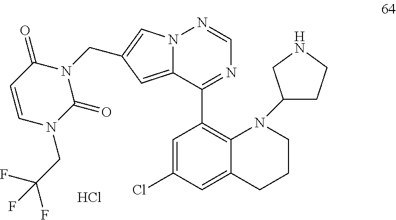 [SMILES: O=c1ccn(CC(F)(F)F)c(=O)n1Cc1cc2c(-c3cc(Cl)cc4c3N(C3CCNC3)CCC4)ncnn2c1 ; Cl]

The title compound (64) was obtained as a hydrochloride salt in 9% overall yield in a similar way to Example 36 with the exception that, in the first step of the synthesis, 1-(2,2,2-trifluoroethyl)pyrimidine-2,4(1H,3H)-dione was used instead of 5,5-dimethylimidazolidine-2,4-dione, and in the second step of the synthesis, the compound 51c was used instead of the compound 30c. In the last step of the synthesis, the crude product was purified by preparative reversed-phase column chromatography (C-18, water+0.3‰ HCl (36%)/MeCN, 99.9:0.1 to 40:60, 40 min, 20 mL/min, Rₜ=25.90 min).

ESI-MS m/z for C₂₆H₂₆ClF₃N₇O₂ found 560.2/562.2 [M+H]⁺; Rₜ=1.29 min (Method A); ¹H NMR (700 MHz, D₂O) δ 8.33 (s, 1H), 7.97 (d, J=1.5 Hz, 1H), 7.64 (d, J=8.0 Hz, 1H), 7.06 (d, J=2.6 Hz, 1H), 6.83 (d, J=2.6 Hz, 1H), 6.46-6.35 (m, 1H), 5.92 (d, J=8.0 Hz, 1H), 5.17-5.04 (m, 2H), 4.60-4.49 (m, 2H), 3.52-3.38 (m, 1H), 3.13-3.01 (m, 1H), 2.99-2.92 (m, 1H), 2.92-2.84 (m, 1H), 2.76-2.48 (m, 4H), 1.84-1.30 (m, 3H).

### Example 65

### Synthesis of 3-((4-(6-chloro-1-(((S)-morpholin-2-yl)methyl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione hydrochloride (65)

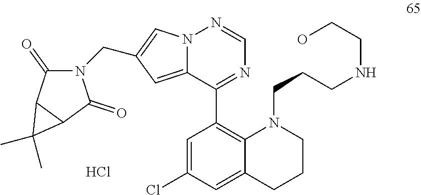

The title compound (65) was obtained as a hydrochloride salt in 3% overall yield in a similar way to Example 51 with the exception that, in the first step of the synthesis, tert-butyl (S)-2-(bromomethyl)morpholine-4-carboxylate was used instead of tert-butyl 3-oxopyrrolidine-1-carboxylate and this step was carried out in the different way (the synthesis of this step was described below) and in the last step of the synthesis, the General Procedure IIIa was used instead of the General Procedure IIIb. The crude product was purified by preparative reversed-phase column chromatography (C-18, water+1‰ HCl (36%)/MeCN, 98:2 to 50:50, 30 min, 20 mL/min, Rₜ=22.1 min).

ESI-MS m/z for C₂₈H₃₂ClN₆O₃ found 535.3/537.3 [M+H]⁺; Rₜ=2.49 min (Method D); ¹H NMR (400 MHz, Methanol-d₄) δ 8.67 (s, 1H), 8.41 (d, J=1.5 Hz, 1H), 7.40 (d, J=1.3 Hz, 2H), 7.26-7.18 (m, 1H), 4.74 (s, 2H), 3.78-3.61 (m, 2H), 3.56-3.43 (m, 1H), 3.29-3.19 (m, 2H), 3.17-3.08 (m, 1H), 2.99-2.76 (m, 5H), 2.72-2.60 (m, 1H), 2.51 (s, 2H), 2.44-2.30 (m, 1H), 2.10-1.95 (m, 1H), 1.95-1.83 (m, 1H), 1.26 (s, 3H), 1.11 (s, 3H).

### Step 1.

### Synthesis of tert-butyl (S)-2-((6-chloro-3,4-dihydroquinolin-1(2H)-yl)methyl)morpholine-4-carboxylate (65a)

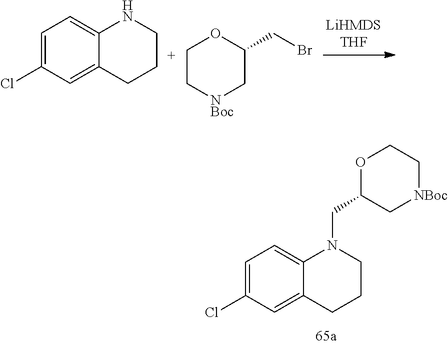

To the cooled to 0° C. solution of 6-chloro-1,2,3,4-tetrahydroquinoline (100 mg; 0.59 mmol) in THE (1 mL) LiHMDS (1 M in THF; 1.2 mL; 1.19 mmol) was added and the resulting mixture was then stirred at this temperature for 1 hour. Then to this mixture a solution of tert-butyl (S)-2-(bromomethyl)morpholine-4-carboxylate (165 mg; 0.59 mmol) in THE (1 mL) was added and the reaction mixture was allowed to warm to room temperature and stirred for 2 hours. Then another portion of LiHMDS (1 M in THF; 0.7 mL; 0.69 mmol) was added and the resulting mixture was then stirred at room temperature overnight. The reaction progress was monitored by LC-MS. When analysis indicated completion of the reaction, water and AcOEt were added and the layers were separated. An aqueous one was neutralized with 2 M HCl till pH 7, then extracted with AcOEt (3×). The combined organic extracts were dried over anhydrous Na₂SO₄, filtered and concentrated in vacuo. The crude product was then purified by flash column chromatography on silica (hexane/AcOEt, 100:0 to 0:100, v/v, 20 min, 20 mL/min). Compound 65a was obtained in 54% yield (118 mg; 0.32 mmol).

ESI-MS m/z for C₁₉H₂₈ClN₂O₃ found 367.1/369.1 [M+H]⁺; Rₜ=1.95 min (Method C); ¹H NMR (400 MHz, CDCl₃) δ 7.01-6.94 (m, 1H), 6.94-6.87 (m, 1H), 6.51-6.44 (m, 1H), 4.03-3.76 (m, 3H), 3.67-3.59 (m, 1H), 3.53-3.44 (m, 1H), 3.40-3.20 (m, 4H), 2.99-2.90 (m, 1H), 2.77-2.59 (m, 3H), 1.97-1.86 (m, 2H), 1.45 (s, 9H).

### Examples 66 and 67

### Synthesis of 3-((4-(6-chloro-1-(pyrrolidin-3-yl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione hydrochloride (66 and 67)

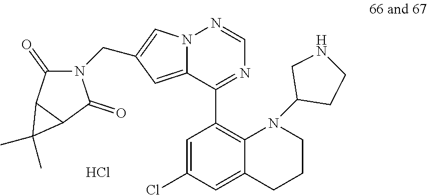 [SMILES: CC1(C)C2C(=O)N(Cc3cc4c(-c5cc(Cl)cc6c5N(C5CCNC5)CCC6)ncnn4c3)C(=O)C21 ; Cl]

The title compounds (66 and 67) were obtained as single enantiomers as hydrochloride salts in the same way as for Example 51 with the exception that, in the third step of the synthesis, two enantiomers were separated by chiral preparative HPLC (Lux-Cellulose-4, Flow 20 mL/min; isocratic resolution n-hexane:iPA; 80:20). Analytical control was carried out by chiral analytical HPLC (Lux-Cellulose-4, Flow 1 mL/min; isocratic resolution n-hexane:iPA; 80:20, Rₜ for 66=27.18 min, Rₜ for 67=34.37 min).

For 66: 100% ee; ESI-MS m/z for C₂₇H₃₀ClN₆O₂ found 505.2/507.2 [M+H]⁺; Rₜ=2.52 min (Method D); ¹H NMR (400 MHz, Methanol-d₄) δ 8.59 (s, 1H), 8.15 (d, J=1.5 Hz, 1H), 7.35 (d, J=2.6 Hz, 1H), 7.31-7.27 (m, 1H), 6.87 (d, J=1.5 Hz, 1H), 4.70-4.64 (m, 2H), 3.87-3.74 (m, 1H), 3.29-3.17 (m, 3H), 2.97-2.72 (m, 4H), 2.49 (s, 2H), 2.37-2.22 (m, 1H), 2.03-1.93 (m, 2H), 1.91-1.77 (m, 1H), 1.52-1.36 (m, 1H), 1.25 (s, 3H), 1.10 (s, 3H).

For 67: 98.2% ee; ESI-MS m/z for C₂₇H₃₀ClN₆O₂ found 505.3/507.3 [M+H]⁺; Rₜ=2.45 min (Method D); ¹H NMR (400 MHz, Methanol-d₄) δ 8.58 (s, 1H), 8.16-8.10 (m, 1H), 7.37-7.33 (m, 1H), 7.31-7.27 (m, 1H), 6.87-6.82 (m, 1H), 4.69-4.65 (m, 2H), 3.88-3.73 (m, 1H), 3.29-3.15 (m, 3H), 2.97-2.72 (m, 4H), 2.49 (s, 2H), 2.38-2.22 (m, 1H), 2.05-1.92 (m, 2H), 1.91-1.75 (m, 1H), 1.51-1.34 (m, 1H), 1.25 (s, 3H), 1.10 (s, 3H).

### Example 68

### Synthesis of 3-((4-(5-chloro-3-methyl-2-(morpholin-2-ylmethyl)phenyl)pyrrolo[2,1-f][1,2,4]triazin-6-yl)methyl)-1-(2,2,2-trifluoroethyl)pyrimidine-2,4(1H,3H)-dione hydrochloride (68)

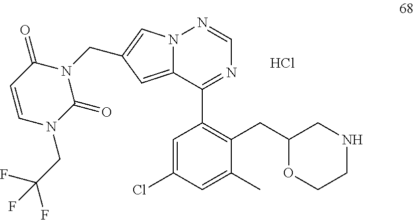 [SMILES: Cc1cc(Cl)cc(-c2ncnn3cc(Cn4c(=O)ccn(CC(F)(F)F)c4=O)cc23)c1CC1CNCCO1 ; Cl]

### Step 1.

### Synthesis of 3-((4-chloropyrrolo[2,1-f][1,2,4]triazin-6-yl)methyl)-1-(2,2,2-trifluoroethyl)pyrimidine-2,4(1H,3H)-dione (68a)

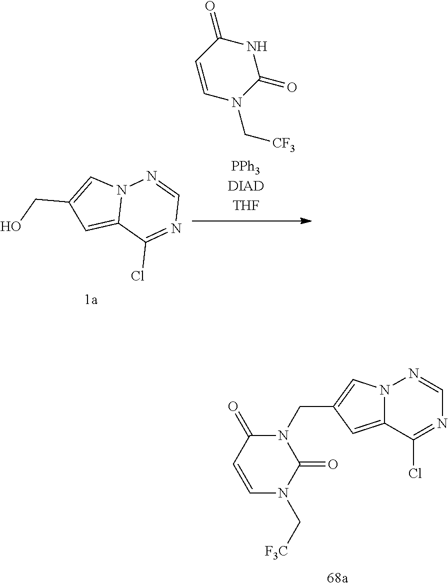 [SMILES: O=c1ccn(CC(F)(F)F)c(=O)[nH]1 ; OCc1cc2c(Cl)ncnn2c1 ; O=c1ccn(CC(F)(F)F)c(=O)n1Cc1cc2c(Cl)ncnn2c1]

To a cooled to −10° C. solution of 1a (181 mg; 0.99 mmol), 1-(2,2,2-trifluoroethyl)pyrimidine-2,4(1H,3H)-dione (230 mg; 1.18 mmol) and PPh₃ (312 mg; 1.19 mmol) in dry THE (3 mL) DIAD (0.25 mL; 1.29 mmol) was added dropwise. The resulting mixture was stirred at −10° C. for 3 hours and then at room temperature for 1 hour. Then the reaction mixture was concentrated in vacuo and the residue was purified by flash column chromatography on silica (hexane/AcOEt, 100:0 to 0:100, v/v, 30 min, 15 mL/min). Compound 68a was obtained in 68% yield (239 mg; 0.67 mmol).

ESI-MS m/z for C₁₃H₁₀ClF₃N₅O₂ found 360.0/362.0 [M+H]⁺; Rₜ=0.86 min (Method A)

### Step 2.

### Synthesis of 3-((4-(5-chloro-2-((4-(4-methoxybenzyl)morpholin-2-yl)methyl)-3-methylphenyl)pyrrolo[2,1-f][1,2,4]triazin-6-yl)methyl)-1-(2,2,2-trifluoroethyl)pyrimidine-2,4(1H,3H)-dione (68b)

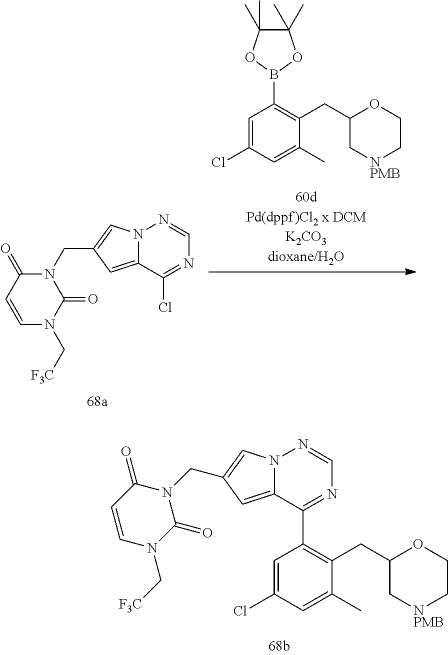

The title compound (68b) was obtained from 68a (23 mg; 0.064 mmol) and from 60d (30 mg; 0.064 mmol) according to the General Procedure IV. The crude product was purified by flash column chromatography on silica (hexane/AcOEt, 100:0 to 0:100, v/v, 25 min, 13 mL/min). Compound 68b was obtained in 37% yield (16 mg; 0.024 mmol).

ESI-MS m/z for C₃₃H₃₃ClF₃N₆O₄ found 669.2/671.2 [M+H]⁺; Rₜ=1.53 min (Method A)

### Step 3.

### Synthesis of 3-((4-(5-chloro-3-methyl-2-(morpholin-2-ylmethyl)phenyl)pyrrolo[2,1-f][1,2,4]triazin-6-yl)methyl)-1-(2,2,2-trifluoroethyl)pyrimidine-2,4(1H,3H)-dione hydrochloride (68)

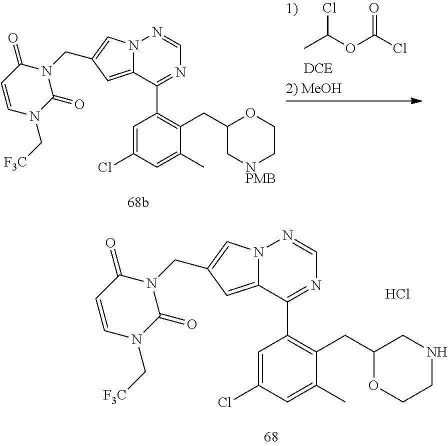

To the cooled to 0° C. solution of 68b (43 mg; 0.064 mmol) in DCE (0.1 mL) 1-chloroethyl carbonochloridate (28 μL; 0.260 mmol) was added dropwise and this mixture was then stirred at 80° C. overnight. The reaction progress was monitored by LC-MS. When analysis indicated completion of the reaction, the solvent was evaporated in vacuo and the residue was dissolved in MeOH (0.5 mL) and a whole was refluxed for 30 minutes. Then the solvent was evaporated in vacuo and the crude product was purified by preparative reversed-phase column chromatography (C-18, water+0.3‰ HCl (36%)/MeCN, 99:1 to 20:80, 30 min, 20 mL/min, Rₜ=15.50 min). The title compound (68) was obtained as a racemate as a hydrochloride salt in 12% yield (5 mg; 0.008 mmol).

ESI-MS m/z for C₂₅H₂₅ClF₃N₆O₃ found 549.4/551.4 [M+H]⁺; Rₜ=1.19 min (Method A); ¹H NMR (700 MHz, Methanol-d₄) δ 8.54 (s, 1H), 8.16 (d, J=1.5 Hz, 1H), 7.61 (d, J=8.0 Hz, 1H), 7.54-7.47 (m, 1H), 7.42-7.35 (m, 1H), 6.77 (d, J=1.5 Hz, 1H), 5.84 (d, J=8.0 Hz, 1H), 5.22 (s, 2H), 4.63-4.58 (m, 2H), 3.75-3.70 (m, 2H), 3.49-3.44 (m, 1H), 3.19-3.13 (m, 1H), 3.11-3.06 (m, 1H), 2.99-2.90 (m, 3H), 2.79-2.71 (m, 1H), 2.48 (s, 3H).

### Example 69

### Synthesis of 3-((4-(1-(azetidin-3-yl)-6-chloro-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione hydrochloride (69)

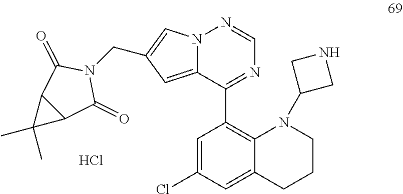 [SMILES: CC1(C)C2C(=O)N(Cc3cc4c(-c5cc(Cl)cc6c5N(C5CNC5)CCC6)ncnn4c3)C(=O)C21 ; Cl]

The title compound (69) was obtained as a hydrochloride salt in 2% overall yield in a similar way to Example 51 with the exception that, in the first step of the synthesis, tert-butyl 3-oxoazetidine-1-carboxylate was used instead of tert-butyl 3-oxopyrrolidine-1-carboxylate and in the last step of the synthesis, the crude product was purified by preparative reversed-phase column chromatography (C-18, water+0.3‰ HCl (36%)/MeCN, 99.9:0.1 to 45:55, 35 min, 20 mL/min, Rₜ=25.40 min).

ESI-MS m/z for C₂₆H₂₈ClN₆O₂ found 491.0/493.0 [M+H]⁺; Rₜ=1.21 min (Method A); ¹H NMR (700 MHz, D₂O) δ 8.47 (d, J=1.3 Hz, 1H), 7.99 (d, J=1.5 Hz, 1H), 7.27-7.19 (m, 1H), 7.02 (d, J=2.5 Hz, 1H), 6.47 (d, J=1.6 Hz, 1H), 4.79 (s, 3H), 4.64 (s, 2H), 4.10-3.71 (m, 2H), 3.46-3.29 (m, 2H), 2.92-2.77 (m, 2H), 2.70-2.59 (m, 2H), 2.04-1.81 (m, 2H), 1.25 (s, 3H), 1.09 (s, 3H).

### Example 70

### Synthesis of 3-((4-(5-chloro-1-((4-fluoropiperidin-4-yl)methyl)-1H-indol-7-yl)pyrrolo[2,1-f][1,2,4]triazin-6-yl)methyl)-1-(2,2,2-trifluoroethyl)pyrimidine-2,4(1H,3H)-dione dihydrochloride (70)

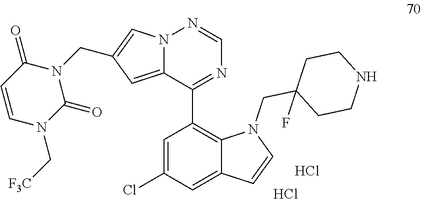 [SMILES: O=c1ccn(CC(F)(F)F)c(=O)n1Cc1cc2c(-c3cc(Cl)cc4ccn(CC5(F)CCNCC5)c34)ncnn2c1 ; Cl ; Cl]

The title compound (70) was obtained as a hydrochloride salt in 28% overall yield in a similar way to Example 10 with the exception that, in the fourth step of the synthesis, the compound 68a was used instead of the compound 1b and in the last step of the synthesis, the crude product was purified by preparative reversed-phase column chromatography (C-18, water+0.3‰ HCl (36%)/MeCN, 98:2 to 30:70, 30 min, 20 mL/min, Rₜ=19.30 min).

ESI-MS m/z for C₂₇H₂₅ClF₄N₇O₂ found 590.4/592.4 [M+H]⁺; Rₜ=1.26 min (Method A); ¹H NMR (700 MHz, Methanol-d₄) δ 8.60 (s, 1H), 8.13 (s, 1H), 7.85 (d, J=2.0 Hz, 1H), 7.62 (d, J=8.0 Hz, 1H), 7.46 (d, J=2.1 Hz, 1H), 7.42-7.35 (m, 1H), 6.82 (s, 1H), 6.72 (d, J=3.3 Hz, 1H), 5.85 (d, J=7.9 Hz, 1H), 5.23 (s, 2H), 4.64-4.58 (m, 2H), 4.42-4.32 (m, 2H), 3.18-3.11 (m, 2H), 2.92-2.84 (m, 2H), 1.71-1.58 (m, 2H), 1.49-1.41 (m, 2H).

### Example 71

### Synthesis of 3-((4-(2-((7-oxa-4-azaspiro[2.5]octan-6-yl)methyl)-5-chloro-3-methylphenyl)pyrrolo[2,1-f][1,2,4]triazin-6-yl)methyl)-6,6-dimethyl-3-azabicyclo[3.1.0]hexane-2,4-dione 2,2,2-trifluoroacetate (71)

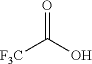

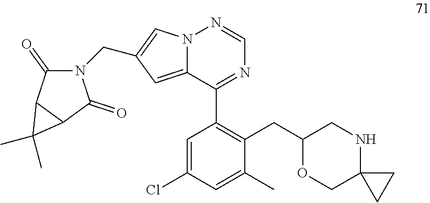 [SMILES: Cc1cc(Cl)cc(-c2ncnn3cc(CN4C(=O)C5C(C4=O)C5(C)C)cc23)c1CC1CNC2(CC2)CO1]

### Step 1.

### Synthesis of (1-((4-methoxybenzyl)amino)cyclopropyl)methanol (71a)

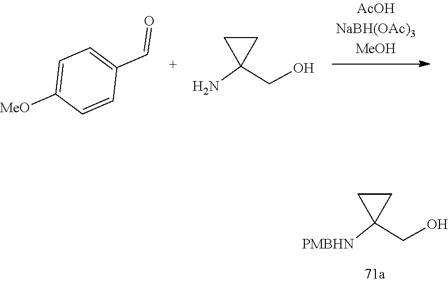

To the solution of (1-aminocyclopropyl)methanol (1.00 g; 11.48 mmol) in MeOH (25 mL) 4-methoxybenzaldehyde (1.40 mL; 11.48 mmol) and glacial acetic acid (AcOH) (0.56 mL) were added and the mixture was stirred for 50 minutes at room temperature. Then sodium triacetoxyborohydride (NaBH(OAc)₃; 2.68 g; 12.63 mmol) was then added in one portion and this mixture was stirred at room temperature overnight. After this time a 0.5 M aqueous solution of NaOH was added and the biphasic mixture was stirred for 30 minutes. The layers were separated and the aqueous layer was additionally extracted with DCM (4×). The combined organic extracts were dried over anhydrous MgSO₄, filtered and the solvents were evaporated in vacuo. The crude product was purified by flash column chromatography on silica (DCM/MeOH, 100:0 to 0:60, v/v, 30 min, 25 mL/min). Compound 71a was obtained in 71% yield (1.69 g; 8.16 mmol).

ESI-MS m/z for C₁₂H₁₈NO₂ found 208.1 [M+H]⁺; Rₜ=0.26 min (Method A)

### Step 2.

### Synthesis of 4-(4-methoxybenzyl)-7-oxa-4-azaspiro[2.5]octan-5-one (71b)

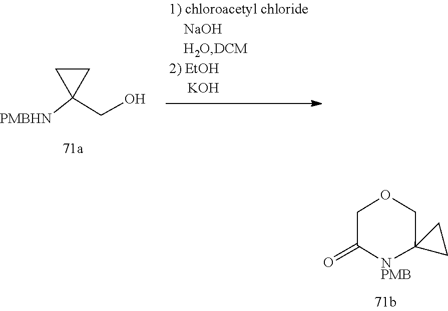

To the solution of 71a (1.68 g; 8.11 mmol) in DCM (10 mL) a solution of NaOH (324 mg; 8.11 mmol) in H₂O (6.5 mL) was added and the mixture was cooled to 0° C. Then to this solution chloroacetyl chloride (0.64 mL; 8.11 mmol) in DCM (2 mL) was added dropwise and the reaction mixture was then stirred at room temperature overnight. After this time, the phases were separated and an organic one was washed with 1 M NaOH (10 mL), 1 M HCl (10 mL) and brine (10 mL) and then was dried over anhydrous MgSO₄, filtered and concentrated in vacuo. The residue was dissolved in absolute EtOH (9 mL) and to this solution KOH (327 mg; 5.82 mmol) was added. The reaction mixture was stirred at room temperature for 4 hours. Then a whole was concentrated in vacuo and the crude product was purified by flash column chromatography on silica (hexane/AcOEt, 100:0 to 0:100, v/v, 20 min, 30 mL/min). Compound 71b was obtained in 50% yield (1 g; 4.05 mmol).

ESI-MS m/z for C₁₄H₁₈NO₃ found 248.0 [M+H]⁺; Rₜ=0.96 min (Method A); ¹H NMR (700 MHz, CDCl₃) δ 7.16-7.07 (m, 2H), 6.88-6.81 (m, 2H), 4.43 (s, 2H), 4.36 (s, 2H), 3.79 (s, 3H), 3.66 (s, 2H), 1.05-0.95 (m, 2H), 0.71-0.61 (m, 2H).

### Step 3.

### Synthesis of 6-(4-chloro-2-iodo-6-methylbenzyl)-4-(4-methoxybenzyl)-7-oxa-4-azaspiro[2.5]octan-5-one 71)

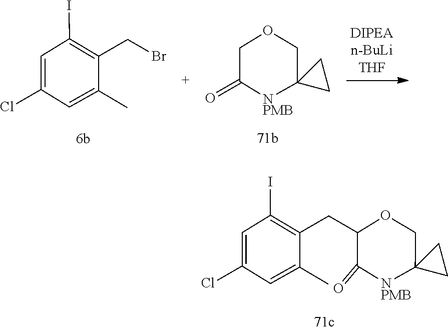

To the cooled to −78° C. solution of DIPEA (0.30 mL; 2.178 mmol) in dry THF (2 mL) n-BuLi (2.5 M in hexane; 0.87 mL; 2.178 mmol) was added dropwise and then the reaction mixture was warmed to room temperature and stirred for 15 minutes. Then the reaction mixture was cooled to −78° C. and to this mixture a solution of 71b (490 mg; 1.980 mmol) in THE (2 mL) was slowly added and the mixture was stirred at this temperature for 1 hour. Then to this mixture a solution of 6b (648 mg; 1.980 mmol) in THE (2 mL) was added and a whole was allowed to warm to room temperature over 1.5 hours. After this time, the reaction mixture was quenched with NH₄Cl (10 mL) and extracted with AcOEt (2×15 mL). The combined organic solutions were dried over anhydrous Na₂SO₄, filtered and concentrated in vacuo. Then the crude product was purified by flash column chromatography on silica (hexane/AcOEt, 100:0 to 0:100, v/v, 25 min, 13 mL/min). Compound 71c was obtained in 83% yield (840 mg; 1.644 mmol).

ESI-MS m/z for C₂₂H₂₄ClINO₃ found 511.9/513.9 [M+H]⁺; Rₜ=1.93 min (Method A); ¹H NMR (700 MHz, CDCl₃) δ 7.77-7.74 (m, 1H), 7.20-7.16 (m, 3H), 6.90-6.87 (m, 2H), 4.76-4.69 (m, 1H), 4.62 (d, J=15.6 Hz, 1H), 4.27-4.20 (m, 1H), 3.82 (s, 3H), 3.80-3.74 (m, 1H), 3.70-3.57 (m, 1H), 3.49 (d, J=11.5 Hz, 1H), 3.38-3.30 (m, 1H), 2.47 (s, 3H), 1.09-0.97 (m, 2H), 0.81-0.76 (m, 1H), 0.70-0.61 (m, 1H).

### Step 4.

### Synthesis of 6-(4-chloro-2-iodo-6-methylbenzyl)-4-(4-methoxybenzyl)-7-oxa-4-azaspiro[2.5]octane (71d)

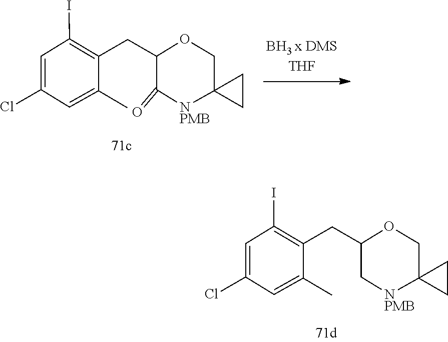

To the cooled to 0° C. solution of 71c (460 mg; 0.899 mmol) in dry THE (8 mL) BH₃×DMS (0.17 mL; 1.180 mmol) was slowly added and this mixture was then stirred at 65° C. for 2 hours. The reaction progress was monitored by LC-MS. When analysis indicated completion of the reaction, to this mixture 6 M HCl (2 mL) was added and whole was stirred at 65° C. for 30 minutes. Then the reaction mixture was cooled to room temperature and alkalized with 4 M NaOH and then extracted with AcOEt (2×50 mL). The combined organic solutions were dried over anhydrous Na₂SO₄, filtered and concentrated in vacuo. The crude product was purified by flash column chromatography on silica (hexane/AcOEt, 100:0 to 0:100, v/v, 20 min, 13 mL/min). Compound 71d was obtained in 80% yield (358 mg; 0.720 mmol).

ESI-MS m/z for C₂₂H₂₆ClINO₂ found 498.2/499.9 [M+H]⁺; Rₜ=1.65 min (Method A)

### Step 5.

### Synthesis of 6-(4-chloro-2-iodo-6-methylbenzyl)-7-oxa-4-azaspiro[2.5]octane (71e)

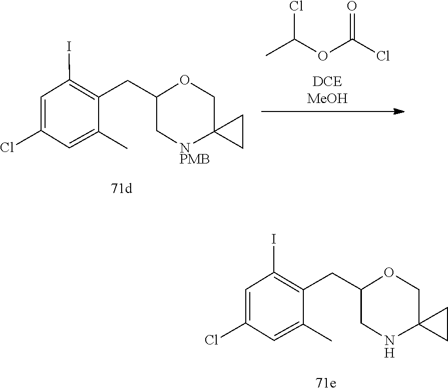

To the cooled to 0° C. solution of 71d (288 mg; 0.578 mmol) in DCE (2 mL) 1-chloroethyl carbonochloridate (0.25 mL; 2.310 mmol) was added and this mixture was then stirred at 80° C. for 6 hours. The reaction progress was monitored by LC-MS. When analysis indicated completion of the reaction, the solvent was evaporated in vacuo and the residue was dissolved in MeOH and a whole was stirred at 70° C. for 30 minutes. Then MeOH was evaporated in vacuo to give the crude product which was used to the next step without any additional purification.

ESI-MS m/z for C₁₄H₁₈ClINO found 377.9/379.8 [M+H]⁺; Rₜ=1.20 min (Method A)

### Step 6.

### Synthesis of tert-butyl 6-(4-chloro-2-iodo-6-methylbenzyl)-7-oxa-4-azaspiro[2.5]octane-4-carboxylate (71f)

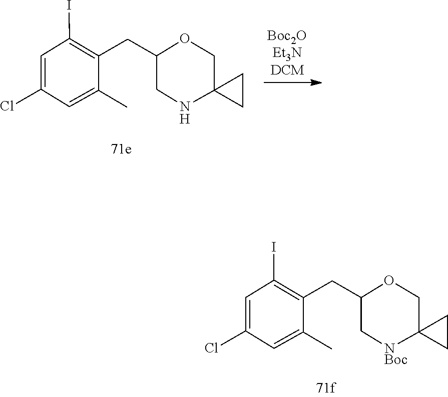 [SMILES: Cc1cc(Cl)cc(I)c1CC1CNC2(CC2)CO1 ; Cc1cc(Cl)cc(I)c1CC1CN(C(=O)OC(C)(C)C)C2(CC2)CO1]

To the solution of 71e (the crude product) in DCM (5 mL) Boc₂O (150 mg; 0.867 mmol) and Et₃N (0.32 mL; 2.312 mmol) were added and this mixture was then stirred at room temperature overnight. The reaction progress was monitored by LC-MS. When analysis indicated completion of the reaction, the solvent was evaporated in vacuo and the residue was then purified by flash column chromatography on silica (hexane/AcOEt, 100:0 to 70:30, v/v, 28 min). Compound 71f was obtained in 72% yield (per two steps)(200 mg; 0.419 mmol).

ESI-MS m/z for C₁₉H₂₆ClINO₃ found 477.9/479.9 [M+H]⁺; Rₜ=2.18 min (Method A); ¹H NMR (700 MHz, CDCl₃) δ 7.70 (d, J=2.2 Hz, 1H), 7.14 (d, J=2.2 Hz, 1H), 4.02-3.86 (m, 2H), 3.74-3.66 (m, 1H), 3.06-2.99 (m, 2H), 2.99-2.92 (m, 1H), 2.88-2.82 (m, 1H), 2.41 (s, 3H), 1.43 (s, 9H), 0.93-0.82 (m, 2H), 0.73-0.68 (m, 1H), 0.65-0.60 (m, 1H).

### Step 7.

### Synthesis of tert-butyl 6-(4-chloro-2-methyl-6-(4,4,5,5-tetramethyl-1,3,2-dioxaborolan-2-yl)benzyl)-7-oxa-4-azaspiro[2.5]octane-4-carboxylate (71g)

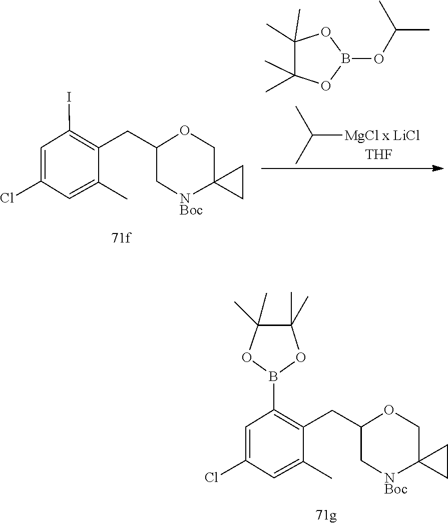

To the cooled to −30° C. solution of 71f (160 mg; 0.335 mmol) in dry THF (3 mL) isopropyl magnesium chloride lithium chloride complex (1.3 M in THF; 0.39 mL; 0.502 mmol) was added dropwise under an Argon atmosphere and the resulting mixture was stirred for 30 minutes. During this time the reaction mixture was allowed to warm to room temperature. Then to this mixture 2-isopropoxy-4,4,5,5-tetramethyl-1,3,2-dioxaborolane (137μ; 0.670 mmol) was added at −30° C. The reaction mixture was allowed to warm to room temperature and stirred for 30 minutes. The reaction progress was monitored by LC-MS. When analysis indicated completion of the reaction, the reaction mixture was quenched with saturated solution of NH₄Cl (10 mL) and a whole was extracted with AcOEt (2×10 mL). The combined organic extracts were washed with brine and dried over anhydrous Na₂SO₄, filtered and concentrated in vacuo. The crude product was used to the next step without any additional purification.

ESI-MS m/z for C₂₀H₃₀BClNO₃ found 377.9/380.1 [M+H-Boc]⁺; Rₜ=2.34 min (Method A)

### Step 8.

### Synthesis of tert-butyl 6-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylbenzyl)-7-oxa-4-azaspiro[2.5]octane-4-carboxylate (71h)

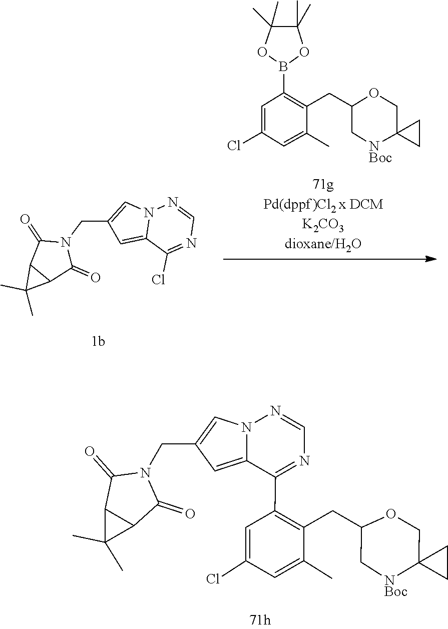 [SMILES: Cc1cc(Cl)cc(B2OC(C)(C)C(C)(C)O2)c1CC1CN(C(=O)OC(C)(C)C)C2(CC2)CO1 ; CC1(C)C2C(=O)N(Cc3cc4c(Cl)ncnn4c3)C(=O)C21 ; Cc1cc(Cl)cc(-c2ncnn3cc(CN4C(=O)C5C(C4=O)C5(C)C)cc23)c1CC1CN(C(=O)OC(C)(C)C)C2(CC2)CO1]

The title compound (71h) was obtained from 1b (31 mg; 0.100 mmol) and from 71g (the crude product) according to the General Procedure IV in 61% yield (per two steps)(38 mg; 0.061 mmol). The crude product was purified by flash column chromatography on silica (hexane/AcOEt, 100:0 to 0:100, v/v, 20 min. 15 mL/min).

ESI-MS m/z for C₃₃H₃₉ClN₅O₅ found 620.2/622.2 [M+H]⁺; Rₜ=1.92 min (Method A)

### Step 9.

### Synthesis of 3-((4-(2-((7-oxa-4-azaspiro[2.5]octan-6-yl)methyl)-5-chloro-3-methylphenyl)pyrrolo[2,1-f][1,2,4]triazin-6-yl)methyl)-6,6-dimethyl-3-azabicyclo[3.1.0]hexane-2,4-dione 2,2,2-trifluoroacetate (71)

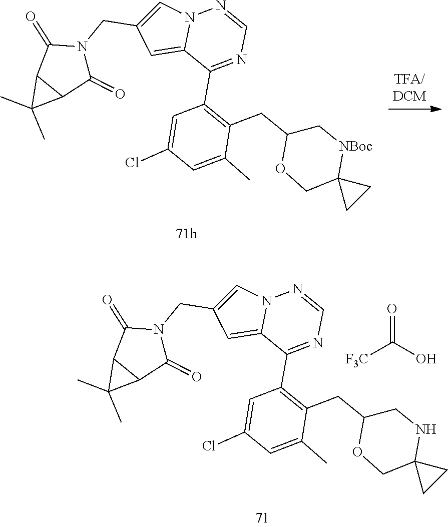

The title compound (71) was obtained as a TFA salt from 71h (35 mg; 0.056 mmol) according to the General Procedure IIIb in 59% yield (21 mg; 0.033 mmol). The crude product was purified by preparative reversed-phase column chromatography (column: Cosmosil Cholester 20×250 mm, water+0.4‰ TFA/MeCN, 99:1 to 20:80, 30 min, 20 mL/min, Rₜ=18.10 min).

ESI-MS m/z for C₂₈H₃₁ClN₅O₃ found 520.4/522.4 [M+H]⁺; Rₜ=1.22 min (Method A); ¹H NMR (700 MHz, Methanol-d₄) δ 8.48 (s, 1H), 7.98 (d, J=1.7 Hz, 1H), 7.46 (d, J=2.4 Hz, 1H), 7.32 (d, J=2.3 Hz, 1H), 6.55 (d, J=2.0 Hz, 1H), 4.68-4.62 (m, 2H), 3.88-3.80 (m, 2H), 3.25-3.20 (m, 1H), 3.11-3.01 (m, 2H), 2.97-2.89 (m, 2H), 2.49 (s, 3H), 2.45 (s, 2H), 1.23 (s, 3H), 1.06 (s, 3H), 0.99-0.94 (m, 2H), 0.88-0.84 (m, 1H), 0.83-0.79 (m, 1H).

### Example 72

### Synthesis of 3-((4-(5-chloro-3-methyl-2-((2,6,6-trimethylmorpholin-2-yl)methyl)phenyl)pyrrolo[2,1-f][1,2,4]triazin-6-yl)methyl)-6,6-dimethyl-3-azabicyclo[3.1.0]hexane-2,4-dione hydrochloride (72)

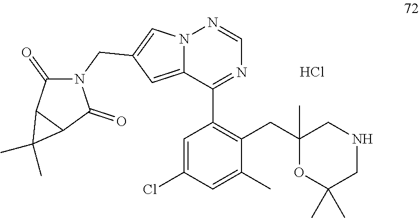 [SMILES: Cc1cc(Cl)cc(-c2ncnn3cc(CN4C(=O)C5C(C4=O)C5(C)C)cc23)c1CC1(C)CNCC(C)(C)O1 ; Cl]

The title compound (72) was obtained as a hydrochloride salt in 0.56% overall yield in a similar way to Example 38 with the exception that, in the first step of the synthesis, 1-amino-2-methylpropan-2-ol was used instead of 3-amino-2,2-difluoropropan-1-ol, in the second step of the synthesis, 2-chloropropanoyl chloride was used instead of chloroacetyl chloride, in the third step of the synthesis, tert-BuLi (1.7 M in pentane) was used instead of n-BuLi (2.5 M in hexane), the fifth step of the synthesis was carried out in the different way (the synthesis of this step was described below) and in the last step of the synthesis, MeOH was used instead of 6 M HCl and the crude product was purified by preparative reversed-phase column chromatography (C-18, water+0.3‰ HCl (36%)/MeCN, 99:1 to 20:80, 30 min, 20 mL/min, Rₜ=18.00 min).

ESI-MS m/z for C₂₉H₃₅ClN₅O₃ found 536.5/538.3 [M+H]⁺; Rₜ=1.45 min (Method A); ¹H NMR (400 MHz, D20, 333K) δ 8.71 (s, 1H), 8.27 (s, 1H), 7.76 (s, 1H), 7.62 (s, 1H), 7.00 (s, 1H), 4.93 (s, 2H), 3.74-3.52 (m, 1H), 3.45-3.26 (m, 3H), 3.13-2.96 (m, 1H), 2.92-2.78 (m, 3H), 2.69 (s, 3H), 1.47 (s, 3H), 1.31 (s, 6H), 1.20 (s, 3H), 0.62 (s, 3H).

### Step 5.

### Synthesis of 2-(4-chloro-2-methyl-6-(4,4,5,5-tetramethyl-1,3,2-dioxaborolan-2-yl)benzyl)-4-(4-methoxybenzyl)-2,6,6-trimethylmorpholine (72e)

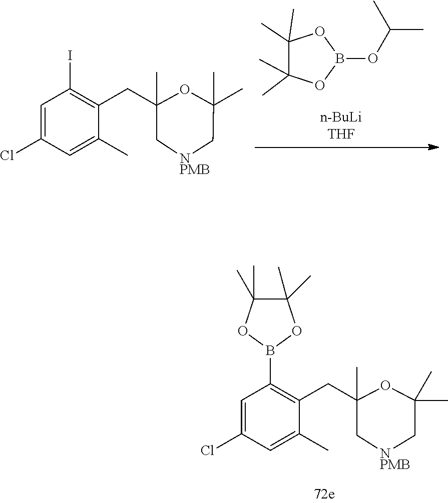

To the cooled to −78° C. solution of 2-(4-chloro-2-iodo-6-methylbenzyl)-4-(4-methoxybenzyl)-2,6,6-trimethylmorpholine (143 mg; 0.28 mmol) in dry THE (2.5 mL) n-BuLi (2.5 M in hexane; 134 μL; 0.33 mmol) was slowly added and after stirring in this temperature for 15 minutes 2-isopropoxy-4,4,5,5-tetramethyl-1,3,2-dioxaborolane (86 μL; 0.42 mmol) was added dropwise and then the reaction mixture was allowed to warm to room temperature and stirred for 1 hour. The reaction progress was monitored by LC-MS. When analysis indicated completion of the reaction, to the reaction mixture a saturated solution of NH₄Cl was added and a whole was extracted with AcOEt (3×). The combined organic layers were washed with brine, dried over anhydrous Na₂SO₄, filtered and concentrated in vacuo. The crude product was then purified by flash column chromatography on silica (hexane/AcOEt, 100:0 to 70:30, v/v, 18 min, 13 mL/min). Compound 72e was obtained in 79% yield (115 mg; 0.22 mmol).

ESI-MS m/z for C₂₉H₄₂BClNO₄ found 514.0/516.0 [M+H]⁺; Rₜ=2.31 min (Method A)

### Example 73

### Synthesis of 3-((4-(5-chloro-3-methyl-2-((piperidin-4-ylmethyl)amino)phenyl)pyrrolo[2,1-f][1,2,4]triazin-6-yl)methyl)-6,6-dimethyl-3-azabicyclo[3.1.0]hexane-2,4-dione dihydrochloride (73)

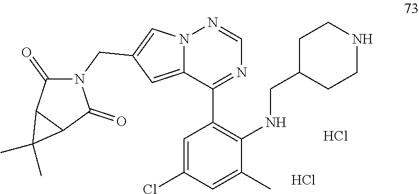 [SMILES: Cc1cc(Cl)cc(-c2ncnn3cc(CN4C(=O)C5C(C4=O)C5(C)C)cc23)c1NCC1CCNCC1 ; Cl ; Cl]

### Step 1.

### Synthesis of tert-butyl 4-(((4-chloro-2-methylphenyl)amino)methyl)piperidine-1-carboxylate (73a)

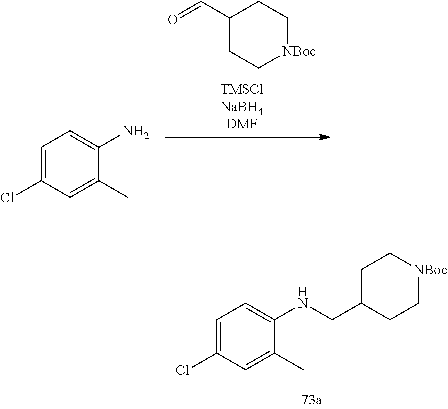

To the solution of 4-chloro-2-methylaniline (200 mg; 1.412 mmol) and tert-butyl 4-formylpiperidine-1-carboxylate (301 mg; 1.412 mmol) in DMF (3 mL) under an Argon atmosphere TMSCl (0.45 mL; 3.530 mmol) was added and the mixture was cooled to 0° C. and then to this mixture NaBH₄ (53 mg; 1.412 mmol) was added and then the reaction mixture was allowed to warm to room temperature and stirred overnight. The reaction progress was monitored by LC-MS. When analysis indicated completion of the reaction, to the reaction mixture an aqueous solution of NaHCO₃ was added and a whole was extracted with AcOEt. The combined organic extracts were dried over anhydrous Na₂SO₄, filtered and the solvents were evaporated in vacuo. The crude product was purified by flash column chromatography on silica (column 12 g: hexane 100%, 2 min, hexane/AcOEt, 100:0 to 0:100, v/v, 15 min, 30 mL/min). Compound 73a was obtained in 53% yield (255 mg; 0.754 mmol).

ESI-MS m/z for C₁₆H₂₃ClN₃O₂ found 324.3/326.3 [M+H-tBu+MeCN]⁺; Rₜ=1.90 min (Method A)

### Step 2.

### Synthesis of tert-butyl 4-(((2-bromo-4-chloro-6-methylphenyl)amino)methyl)piperidine-1-carboxylate (73b)

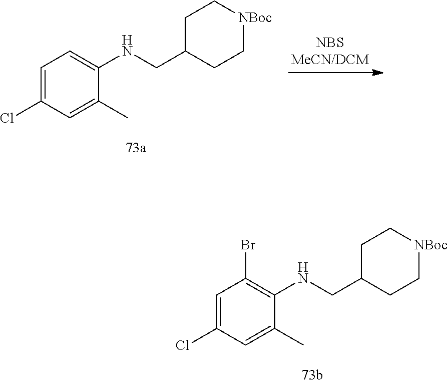 [SMILES: Cc1cc(Cl)ccc1NCC1CCN(C(=O)OC(C)(C)C)CC1 ; Cc1cc(Cl)cc(Br)c1NCC1CCN(C(=O)OC(C)(C)C)CC1]

To the solution of 73a (255 mg; 0.754 mmol) in MeCN/DCM (2 mL; 1:1, v/v) NBS (161 mg; 0.905 mmol) was added at 0° C. and the resulting mixture was allowed to warm to room temperature over an hour. The reaction progress was monitored by LC-MS. When analysis indicated completion of the reaction, to the reaction mixture an aqueous solution of NaHCO₃ was added and a whole was extracted with AcOEt. The combined organic extracts were dried over anhydrous Na₂SO₄, filtered and concentrated in vacuo. The crude product was purified by flash column chromatography on silica (hexane/AcOEt, 100:0 to 40:60, v/v, 22 min, 40 mL/min). Compound 73b was obtained in 81% yield (253 mg; 0.608 mmol).

ESI-MS m/z for C₁₄H₁₉BrClN₂O₂ found 361.0/363.0 [M+H-tBu]⁺; Rₜ=2.15 min (Method A)

### Step 3.

### Synthesis of (2-(((1-(tert-butoxycarbonyl)piperidin-4-yl)methyl)amino)-5-chloro-3-methylphenyl)boronic acid (73c)

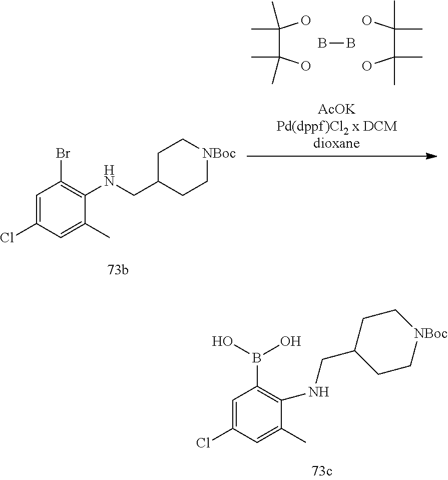

In a Schlenk flask was placed the compound 73b (100 mg; 0.240 mmol), bis(pinacolato)diboron (122 mg; 0.480 mmol), AcOK (71 mg; 0.720 mmol), Pd(dppf)Cl₂×DCM (19 mg; 0.024 mmol) and dioxane (2 mL). The reaction mixture was intensively flushed with Ar. The mixture was capped and stirred at 90° C. overnight. The reaction progress was monitored by LC-MS. When analysis indicated completion of the reaction, the reaction mixture was concentrated in vacuo and the crude product was used to the next step without any additional purification.

ESI-MS m/z for C₁₈H₂₉BClN₂O₄ found 383.0/385.0 [M+H]⁺; Rₜ=1.38 min (Method A)

### Step 4.

### Synthesis of tert-butyl 4-(((4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenyl)amino)methyl)piperidine-1-carboxylate (73d)

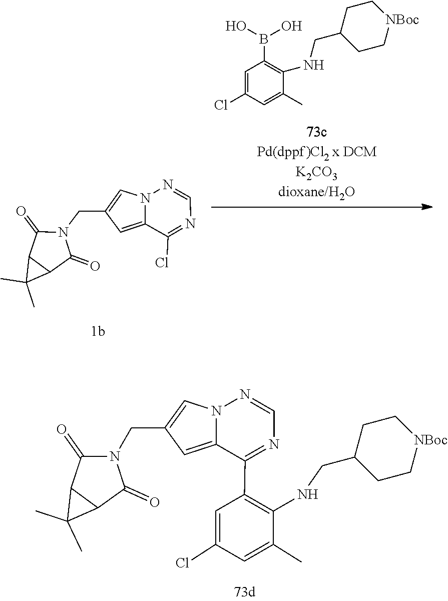 [SMILES: Cc1cc(Cl)cc(B(O)O)c1NCC1CCN(C(=O)OC(C)(C)C)CC1 ; CC1(C)C2C(=O)N(Cc3cc4c(Cl)ncnn4c3)C(=O)C21 ; Cc1cc(Cl)cc(-c2ncnn3cc(CN4C(=O)C5C(C4=O)C5(C)C)cc23)c1NCC1CCN(C(=O)OC(C)(C)C)CC1]

The title compound (73d) was obtained from 1b (123 mg; 0.40 mmol) and from 73c (the crude product) according to the General Procedure IV in 29% yield (per two steps)(44 mg; 0.07 mmol).

ESI-MS m/z for C₃₂H₄₀ClN₆O₄ found 607.3/609.3 [M+H]⁺; Rₜ=1.96 min (Method A)

### Step 5.

### Synthesis of 3-((4-(5-chloro-3-methyl-2-((piperidin-4-ylmethyl)amino)phenyl)pyrrolo[2,1-f][1,2,4]triazin-6-yl)methyl)-6,6-dimethyl-3-azabicyclo[3.1.0]hexane-2,4-dione dihydrochloride (73)

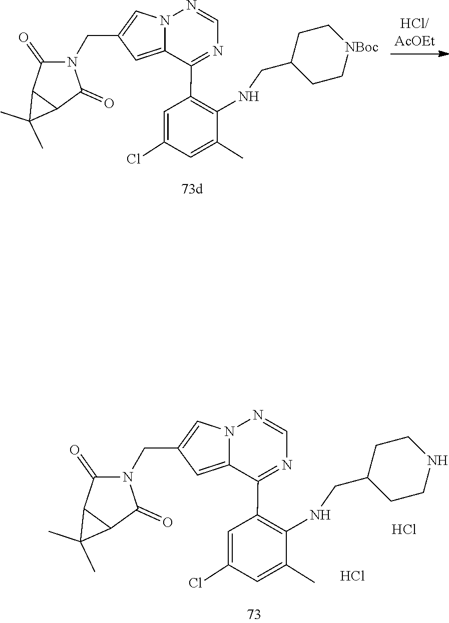 [SMILES: Cc1cc(Cl)cc(-c2ncnn3cc(CN4C(=O)C5C(C4=O)C5(C)C)cc23)c1NCC1CCN(C(=O)OC(C)(C)C)CC1 ; Cc1cc(Cl)cc(-c2ncnn3cc(CN4C(=O)C5C(C4=O)C5(C)C)cc23)c1NCC1CCNCC1 ; Cl ; Cl]

The title compound (73) was obtained as a dihydrochloride salt from 73d (44 mg; 0.07 mmol) according to the General Procedure IIIa in 28% yield (12 mg; 0.02 mmol). The crude product was purified by preparative reversed-phase column chromatography (C-18, water+0.4‰ HCl (36%)/MeCN, 98:2 to 30:70, 30 min, 20 mL/min, Rₜ=18.33 min).

ESI-MS m/z for C₂₇H₃₂ClN₆O₂ found 507.5/509.5 [M+H]⁺; Rₜ=1.25 min (Method A); ¹H NMR (700 MHz, D₂O) δ 8.54 (s, 1H), 8.15 (s, 1H), 7.51 (d, J=1.9 Hz, 1H), 7.42 (d, J=2.0 Hz, 1H), 7.01 (s, 1H), 4.74 (s, 2H), 3.37-3.29 (m, 2H), 2.83-2.74 (m, 4H), 2.63 (s, 2H), 2.41 (s, 3H), 1.72-1.65 (m, 1H), 1.65-1.58 (m, 2H), 1.26 (s, 3H), 1.23-1.14 (m, 2H), 1.05 (s, 3H).

### Example 74 and 75

### Synthesis of 3-((4-(5-chloro-3-methyl-2-((3-methylpiperidin-3-yl)methoxy)phenyl)pyrrolo[2,1-f][1,2,4]triazin-6-yl)methyl)-6,6-dimethyl-3-azabicyclo[3.1.0]hexane-2,4-dione hydrochloride (74 and 75)

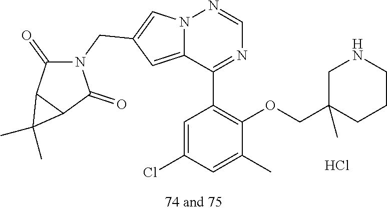 [SMILES: Cc1cc(Cl)cc(-c2ncnn3cc(CN4C(=O)C5C(C4=O)C5(C)C)cc23)c1OCC1(C)CCCNC1 ; Cl]

The title compounds (74 and 75) were obtained as single enantiomers as hydrochloride salts in 2% overall yield (for 74) and in 2% overall yield (for 75) in a similar way to Example 48 with the exception that, in the first step of the synthesis, tert-butyl 3-(hydroxymethyl)-3-methylpiperidine-1-carboxylate was used instead of tert-butyl 4-(hydroxymethyl)piperidine-1-carboxylate. In the third step of the synthesis, the crude products mixture (racemate) was purified by flash column chromatography on silica (hexane/AcOEt, 100:0 to 40:60, v/v, 35 min, 13 mL/min) and then the single enantiomers were separated by chiral preparative HPLC (Amylose-2, 5 μm Lux, 250×21.2 mm, Flow 15 mL/min; isocratic resolution n-hexane:iPA; 85:15; 40 min). Analytical control was carried out by chiral analytical HPLC (Amylose-2, 2.5 μm Lux, 150×4.6 mm, Flow 1 mL/min; n-hexane:iPA; 90:10 to 10:90, 40 min, Rₜ for 74=13.28 min, Rₜ for 75=15.37 min). In the last step of the synthesis, both isomers were converted into products in separate reactions, in which both the crude products were purified by preparative reversed-phase column chromatography (column: Cosmosil Cholester 20×250 mm, water+0.3‰ HCl (36%)/MeCN, 98:2 to 40:60, 30 min, 20 mL/min, Rₜ=20.08 min (for both isomers)). Analytical control was carried out by chiral analytical HPLC (Amylose-2, 2.5 μm Lux, 150×4.6 mm, Flow 1 mL/min; n-hexane:iPA; 90:10 to 10:90, 40 min, Rₜ for 74=22.12 min, Rₜ for 75=23.37 min).

For 74: % ee; ESI-MS m/z for C₂₈H₃₃ClN₅O₃ found 522.4/524.3 [M+H]⁺; Rₜ=1.30 min (Method A); ¹H NMR (700 MHz, Methanol-d₄) δ 8.62 (s, 1H), 8.25 (d, J=1.5 Hz, 1H), 7.60-7.58 (m, 1H), 7.49 (d, J=2.6 Hz, 1H), 6.99 (d, J=1.4 Hz, 1H), 4.71 (s, 2H), 3.47 (d, J=9.1 Hz, 1H), 3.40 (d, J=9.1 Hz, 1H), 3.18-3.11 (m, 1H), 2.91-2.85 (m, 1H), 2.82 (d, J=12.7 Hz, 1H), 2.74 (d, J=12.7 Hz, 1H), 2.49 (s, 2H), 2.42 (s, 3H), 1.78-1.69 (m, 1H), 1.67-1.58 (m, 1H), 1.46-1.39 (m, 1H), 1.29-1.21 (m, 4H), 1.11 (s, 3H), 0.89 (s, 3H).

For 75: % ee; ESI-MS m/z for C₂₈H₃₃ClN₅O₃ found 522.4/524.3 [M+H]⁺; Rₜ=1.30 min (Method A); ¹H NMR (700 MHz, Methanol-d₄) δ 8.54 (s, 1H), 8.07 (d, J=1.5 Hz, 1H), 7.54-7.49 (m, 1H), 7.42 (d, J=2.6 Hz, 1H), 6.77 (d, J=1.5 Hz, 1H), 4.68 (s, 2H), 3.45 (d, J=9.1 Hz, 1H), 3.40 (d, J=9.1 Hz, 1H), 3.16-3.10 (m, 1H), 2.90-2.84 (m, 1H), 2.78 (d, J=12.7 Hz, 1H), 2.71 (d, J=12.8 Hz, 1H), 2.48 (s, 2H), 2.41 (s, 3H), 1.73-1.66 (m, 1H), 1.64-1.57 (m, 1H), 1.43-1.38 (m, 1H), 1.24 (s, 3H), 1.23-1.18 (m, 1H), 1.09 (s, 3H), 0.85 (s, 3H).

### Example 76 and 77

### Synthesis of 3-((4-(5-chloro-3-methyl-2-((R)-1-((S)-morpholin-2-yl)ethoxy)phenyl)pyrrolo[2,1-f][1,2,4]triazin-6-yl)methyl)-6,6-dimethyl-3-azabicyclo[3.1.0]hexane-2,4-dione hydrochloride (76 and 77)

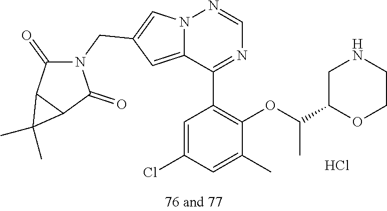 [SMILES: Cc1cc(Cl)cc(-c2ncnn3cc(CN4C(=O)C5C(C4=O)C5(C)C)cc23)c1OC(C)[C@@H]1CNCCO1 ; Cl]

The title compounds (76 and 77) were obtained as single enantiomers as hydrochloride salts in 6% overall yield (for 76) and in 7% overall yield (for 77) in a similar way to Example 48 with the exception that, in the first step of the synthesis, tert-butyl (S)-2-((S)-1-hydroxyethyl)morpholine-4-carboxylate was used instead of tert-butyl 4-(hydroxymethyl)piperidine-1-carboxylate. In the third step of the synthesis, the crude products mixture (racemate) was purified by flash column chromatography on silica (hexane/AcOEt, 100:0 to 30:70, v/v, 40 min, 13 mL/min) and then the single enantiomers were separated by chiral preparative HPLC (Amylose-2, 5 μm Lux, 250×21.2 mm, Flow 15 mL/min; isocratic resolution n-hexane:iPA; 80:20; 40 min). Analytical control was carried out by chiral analytical HPLC (Amylose-2, 2.5 μm Lux, 150×4.6 mm, Flow 1 mL/min; n-hexane:iPA; 90:10 to 10:90, 40 min, Rₜ for 76=14.90 min, Rₜ for 77=19.00 min). In the last step of the synthesis, both isomers were converted into products in separated reactions, in which both the crude products were purified by preparative reversed-phase column chromatography (column: Cosmosil Cholester 20×250 mm, water+0.3‰ HCl (36%)/MeCN, 98:2 to 40:60, 30 min, 20 mL/min, Rₜ=19.80 min (for 76) and Rₜ=20.10 min (for 76)). Analytical control was carried out by chiral analytical HPLC (Amylose-2, 2.5 μm Lux, 150×4.6 mm, Flow 1 mL/min; n-hexane:iPA; 90:10 to 10:90, 40 min, Rₜ for 76=23.80 min, Rₜ for 77=24.60 min).

For 76: ESI-MS m/z for C₂₇H₃₁ClN₅O₄ found 524.2/526.1 [M+H]⁺; Rₜ=1.28 min (Method A); ¹H NMR (700 MHz, Methanol-d₄) δ 8.59 (s, 1H), 8.19 (d, J=1.5 Hz, 1H), 7.56-7.53 (m, 1H), 7.46 (d, J=2.7 Hz, 1H), 6.95 (d, J=1.5 Hz, 1H), 4.72 (s, 2H), 4.01-3.93 (m, 1H), 3.85-3.78 (m, 1H), 3.58-3.51 (m, 2H), 3.21-3.13 (m, 1H), 3.06-2.98 (m, 1H), 2.96-2.90 (m, 2H), 2.50 (s, 2H), 2.39 (s, 3H), 1.24 (s, 3H), 1.09 (s, 3H), 0.88 (d, J=6.5 Hz, 3H).

For 77: ESI-MS m/z for C₂₇H₃₁ClN₅O₄ found 524.2/526.1 [M+H]⁺; Rₜ=1.28 min (Method A); ¹H NMR (700 MHz, Methanol-d₄) δ 8.58 (s, 1H), 8.16 (d, J=1.5 Hz, 1H), 7.57-7.54 (m, 1H), 7.47 (d, J=2.6 Hz, 1H), 6.93-6.90 (m, 1H), 4.76-4.68 (m, 2H), 3.88-3.76 (m, 2H), 3.55-3.46 (m, 2H), 3.25-3.20 (m, 1H), 3.18-3.13 (m, 1H), 2.98-2.90 (m, 1H), 2.73-2.66 (m, 1H), 2.50 (s, 2H), 2.40 (s, 3H), 1.24 (s, 3H), 1.09 (s, 3H), 0.94 (d, J=6.5 Hz, 3H).

### Example 78

### Synthesis of 3-((4-(5-chloro-3-methyl-2-(((4-methylpiperidin-4-yl)methyl)amino)phenyl)pyrrolo[2,1-f][1,2,4]triazin-6-yl)methyl)-6,6-dimethyl-3-azabicyclo[3.1.0]hexane-2,4-dione dihydrochloride (78)

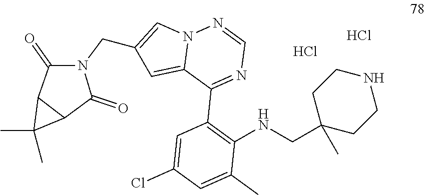 [SMILES: Cc1cc(Cl)cc(-c2ncnn3cc(CN4C(=O)C5C(C4=O)C5(C)C)cc23)c1NCC1(C)CCNCC1 ; Cl ; Cl]

The title compound (78) was obtained as a dihydrochloride salt in 5% overall yield in a similar way to Example 73 with the exception that, in the first step of the synthesis, tert-butyl 4-formyl-4-methylpiperidine-1-carboxylate was used instead of tert-butyl 4-formylpiperidine-1-carboxylate and in the last step of the synthesis, the crude product was purified by preparative reversed-phase column chromatography (C-18, water+0.4‰ HCl (36%)/MeCN, 98:2 to 30:70, 30 min, 20 mL/min, Rₜ=19.53 min).

ESI-MS m/z for C₂₈H₃₄ClN₆O₂ found 521.5/523.5 [M+H]⁺; Rₜ=1.36 min (Method A); ¹H NMR (700 MHz, D₂O) δ 8.53 (s, 1H), 8.12 (s, 1H), 7.44 (d, J=2.0 Hz, 1H), 7.33 (d, J=2.2 Hz, 1H), 6.95 (s, 1H), 4.72 (s, 2H), 3.11-3.02 (m, 4H), 2.65-2.58 (m, 4H), 2.40 (s, 3H), 1.47-1.38 (m, 4H), 1.25 (s, 3H), 1.03 (s, 3H), 0.86 (s, 3H).

### Example 79

### Synthesis of 3-((4-(5-chloro-3-methyl-2-((piperidin-3-ylmethyl)amino)phenyl)pyrrolo[2,1-f][1,2,4]triazin-6-yl)methyl)-6,6-dimethyl-3-azabicyclo[3.1.0]hexane-2,4-dione dihydrochloride (79)

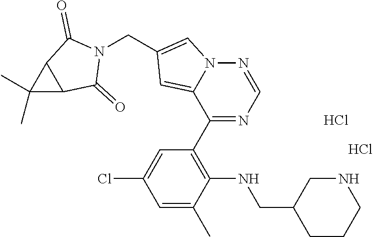 [SMILES: Cc1cc(Cl)cc(-c2ncnn3cc(CN4C(=O)C5C(C4=O)C5(C)C)cc23)c1NCC1CCCNC1 ; Cl ; Cl]

The title compound (79) was obtained as a dihydrochloride salt in 7% overall yield in a similar way to Example 73 with the exception that, in the first step of the synthesis, tert-butyl 3-formylpiperidine-1-carboxylate and NaBH(OAc)₃ were used instead of tert-butyl 4-formylpiperidine-1-carboxylate and NaBH₄, and in the last step of the synthesis, the crude product was purified by preparative reversed-phase column chromatography (C-18, water+0.4‰ HCl (36%)/MeCN, 98:2 to 30:70, 30 min, 20 mL/min, Rₜ=18.20 min).

ESI-MS m/z for C₂₇H₃₂ClN₆O₂ found 507.5/509.5 [M+H]⁺; Rₜ=1.26 min (Method A); ¹H NMR (700 MHz, D₂O) δ 8.52 (s, 1H), 8.10 (d, J=1.2 Hz, 1H), 7.44 (d, J=2.2 Hz, 1H), 7.28 (d, J=2.4 Hz, 1H), 6.87 (s, 1H), 4.73 (s, 2H), 3.33-3.25 (m, 1H), 3.09-3.03 (m, 1H), 2.79-2.72 (m, 1H), 2.67-2.61 (m, 3H), 2.60-2.54 (m, 1H), 2.42-2.37 (m, 1H), 2.36 (s, 3H), 1.80-1.73 (m, 1H), 1.73-1.66 (m, 1H), 1.46-1.37 (m, 1H), 1.37-1.31 (m, 1H), 1.25 (s, 3H), 1.03 (s, 3H), 0.94-0.84 (m, 1H).

### Example 80

### Synthesis of 3-((4-(5-chloro-2-(((4-fluoropiperidin-4-yl)methyl)amino)-3-methylphenyl)pyrrolo[2,1-f][1,2,4]triazin-6-yl)methyl)-6,6-dimethyl-3-azabicyclo[3.1.0]hexane-2,4-dione dihydrochloride (80)

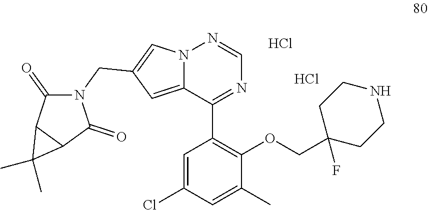 [SMILES: Cc1cc(Cl)cc(-c2ncnn3cc(CN4C(=O)C5C(C4=O)C5(C)C)cc23)c1OCC1(F)CCNCC1 ; Cl ; Cl]

The title compound (80) was obtained as a dihydrochloride salt in 7% overall yield in a similar way to Example 10 with the exception that, in the first step of the synthesis 2-bromo-4-chloro-6-methylaniline was used instead of 7-bromo-5-chloro-1H-indole and in the last step of the synthesis, the crude product was purified by preparative reversed-phase column chromatography (C-18, water+0.4‰ HCl (36%)/MeCN, 98:2 to 30:70, 30 min, 20 mL/min, Rₜ=18.80 min).

ESI-MS m/z for C₂₇H₃₁ClFN₆O₂ found 525.4/527.4 [M+H]⁺; Rₜ=1.30 min (Method A); ¹H NMR (700 MHz, D₂O) δ 8.40 (s, 1H), 8.02 (d, J=1.5 Hz, 1H), 7.33-7.25 (m, 1H), 7.12 (d, J=2.5 Hz, 1H), 6.82 (d, J=1.5 Hz, 1H), 4.59 (s, 2H), 3.18-3.11 (m, 2H), 2.96-2.91 (m, 2H), 2.85-2.72 (m, 2H), 2.49 (s, 2H), 2.25 (s, 3H), 1.83-1.67 (m, 2H), 1.52-1.41 (m, 2H), 1.13 (s, 4H), 0.92 (s, 3H).

### Example 81

### Synthesis of 3-((4-(5-chloro-3-methyl-2-((((S)-morpholin-2-yl)methyl)amino)phenyl)pyrrolo[2,1-f][1,2,4]triazin-6-yl)methyl)-6,6-dimethyl-3-azabicyclo[3.1.0]hexane-2,4-dione dihydrochloride (81)

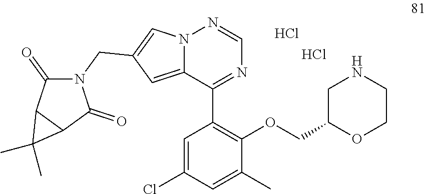 [SMILES: Cc1cc(Cl)cc(-c2ncnn3cc(CN4C(=O)C5C(C4=O)C5(C)C)cc23)c1OC[C@@H]1CNCCO1 ; Cl ; Cl]

### Step 1.

### Synthesis of tert-butyl (S)-2-((4-chloro-2-methylphenyl)carbamoyl)morpholine-4-carboxylate (81a)

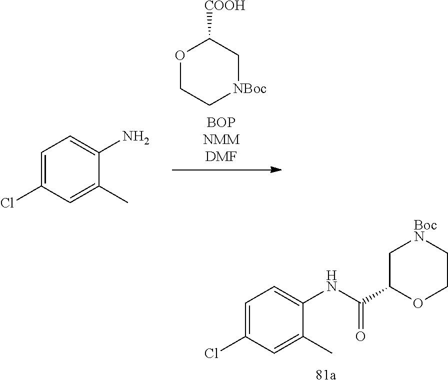

The solution of (S)-4-(tert-butoxycarbonyl)morpholine-2-carboxylic acid (100 mg; 0.432 mmol), 4-chloro-2-methylaniline (61 mg; 432 mmol), N-methylmorpholine (NMM; 190 μL; 1.730 mmol) and BOP (benzotriazol-1-yloxytris(dimethylamino)phosphonium hexafluorophosphate)(230 mg; 518 mmol) in dry DMF (3 mL) was stirred at room temperature overnight. The reaction progress was monitored by LC-MS. When analysis indicated completion of the reaction, to the reaction mixture water and AcOEt were added. The phases were separated and an aqueous one was extracted with AcOEt (2×). The combined organic extracts were dried over anhydrous Na₂SO₄, filtered and concentrated in vacuo. The crude product was purified by flash column chromatography on silica (hexane/AcOEt, 100:0 to 0:100, v/v, 17 min, 13 mL/min). Compound 81a was obtained in 85% yield (130 mg; 0.367 mmol).

ESI-MS m/z for C₁₃H₁₆ClN₂O₄ found 299.0/301.01.56 [M+H-tBu]⁺; Rₜ=1.56 min (Method A)

### Step 2.

### Synthesis of tert-butyl (R)-2-(((4-chloro-2-methylphenyl)amino)methyl)morpholine-4-carboxylate (81b)

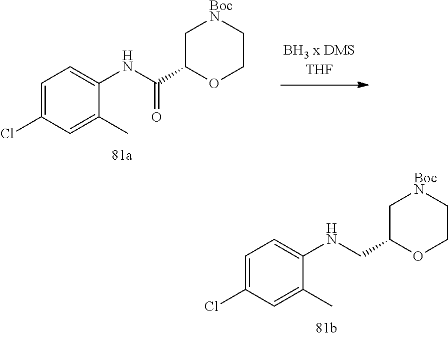 [SMILES: Cc1cc(Cl)ccc1NC(=O)[C@@H]1CN(C(=O)OC(C)(C)C)CCO1 ; Cc1cc(Cl)ccc1NC[C@@H]1CN(C(=O)OC(C)(C)C)CCO1]

To the cooled to 0° C. solution of 81a (100 mg; 0.282 mmol) in dry THF (1.7 mL) BH₃×DMS (2 M in THF; 0.30 mL; 0.592 mmol) was slowly added and this mixture was then stirred at 40° C. for 4 hours in a capped ampule. The reaction progress was monitored by LC-MS. When analysis indicated completion of the reaction, to this mixture 6 M HCl (1 mL) was added and a whole was refluxed for 1 hour. Then the reaction mixture was cooled to room temperature and alkalized with 4 M NaOH to reach pH 9 and then extracted with AcOEt (3×30 mL). The combined organic solutions were dried over anhydrous Na₂SO₄, filtered and concentrated in vacuo. The crude product was purified by flash column chromatography on silica (column 12 g, hexane/AcOEt, 100:0 to 40:60, v/v, 20 min, 30 mL/min). Compound 81b was obtained in 80% yield (77 mg; 0.226 mmol).

ESI-MS m/z for C₁₇H₂₆ClN₂O₃ found 341.3/343.3 [M+H]⁺; Rₜ=1.76 min (Method A)

### Step 3.

### Synthesis of tert-butyl (R)-2-(((2-bromo-4-chloro-6-methylphenyl)amino)methyl)morpholine-4-carboxylate (81c)

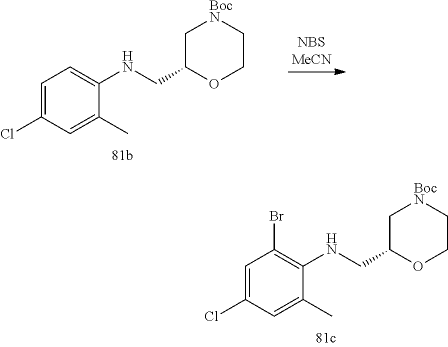 [SMILES: Cc1cc(Cl)ccc1NC[C@@H]1CN(C(=O)OC(C)(C)C)CCO1 ; Cc1cc(Cl)cc(Br)c1NC[C@@H]1CN(C(=O)OC(C)(C)C)CCO1]

To the solution of 81b (100 mg; 0.293 mmol) in MeCN (2 mL) NBS (63 mg; 0.352 mmol) was added in one portion at 0° C. and the resulting mixture was stirred at this temperature for 3 hours under an Argon atmosphere. The reaction progress was monitored by LC-MS. When analysis indicated completion of the reaction, to the reaction mixture an aqueous solution of NaHCO₃ was added and a whole was extracted with AcOEt (2×). The combined organic extracts were dried over anhydrous Na₂SO₄, filtered and concentrated in vacuo. The crude product was used to the next step without any additional purification. Compound 81b was obtained in 89% yield (110 mg; 0.262 mmol).

ESI-MS m/z for C₁₇H₂₅BrClN₂O₃ found 419.0/421.0 [M+H]⁺; Rₜ=1.95 min (Method A)

### Step 4.

### Synthesis of (R)-(2-(((4-(tert-butoxycarbonyl)morpholin-2-yl)methyl)amino)-5-chloro-3-methylphenyl)boronic acid (81d)

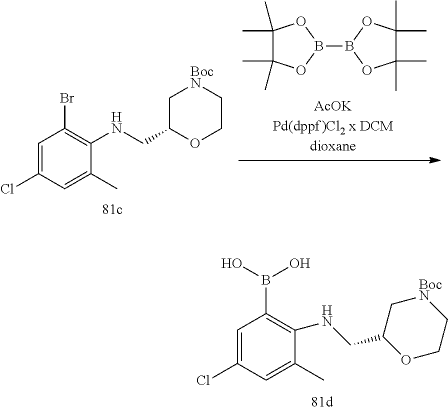

In an ampule flask was placed the compound 81c (the crude product), bis(pinacolato)diboron (125 mg; 0.476 mmol), AcOK (71 mg; 0.720 mmol), Pd(dppf)Cl₂×DCM (9.9 mg; 0.012 mmol) and dioxane (1.5 mL). The reaction mixture was intensively flushed with Ar. The mixture was capped and stirred at 90° C. overnight. The reaction progress was monitored by LC-MS. When analysis indicated completion of the reaction, the reaction mixture was concentrated in vacuo and the crude product was used to the next step without any additional purification.

ESI-MS m/z for C₁₇H₂₇BClN₂O₅ found 385.0/387.0 [M+H]⁺; Rₜ=1.49 min (Method A)

### Step 5.

### Synthesis of tert-butyl (2R)-2-(((4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenyl)amino)methyl)morpholine-4-carboxylate (81e)

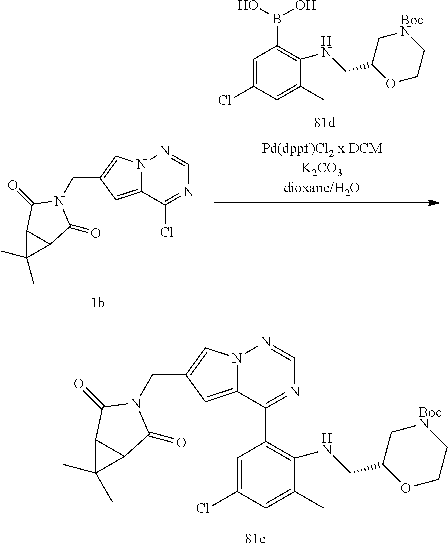

The title compound (81e) was obtained from 1b (72 mg; 0.238 mmol) and from 81d (the crude product) according to the General Procedure IV in 14% yield (per two steps)(20 mg; 0.033 mmol).

ESI-MS m/z for C₃₁H₃₈ClN₆O₅ found 609.0/611.0 [M+H]⁺; Rₜ=1.83 min (Method A)

### Step 6.

### Synthesis of 3-((4-(5-chloro-3-methyl-2-((((S)-morpholin-2-yl)methyl)amino)phenyl)pyrrolo[2,1-f][1,2,4]triazin-6-yl)methyl)-6,6-dimethyl-3-azabicyclo[3.1.0]hexane-2,4-dione dihydrochloride (81)

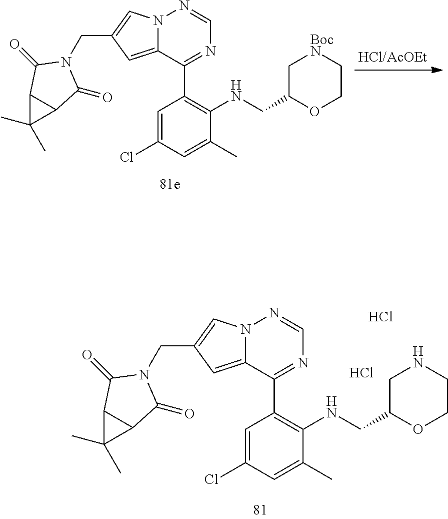

The title compound (81) was obtained as a dihydrochloride salt from 81e (20 mg; 0.033 mmol) according to the General Procedure IIIa in 36% yield (7 mg; 0.012 mmol). The crude product was purified by preparative reversed-phase column chromatography (C-18, water+0.4‰ HCl (36%)/MeCN, 98:2 to 30:70, 30 min, 20 mL/min, Rₜ=18.11 min).

ESI-MS m/z for C₂₆H₃₀ClN₆O₃ found 509.4/511.4 [M+H]⁺; Rₜ=1.17 min (Method A); ¹H NMR (700 MHz, D₂O) δ 8.53 (s, 1H), 8.11 (s, 1H), 7.50 (s, 1H), 7.43 (d, J=2.1 Hz, 1H), 6.96 (s, 1H), 4.76-4.74 (m, 2H), 3.46-3.35 (m, 2H), 3.10-3.02 (m, 3H), 2.91-2.80 (m, 3H), 2.70-2.61 (m, 3H), 2.39 (s, 3H), 1.27 (s, 3H), 1.08 (s, 3H).

### Example 82

### Synthesis of (R)-3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione hydrochloride (82)

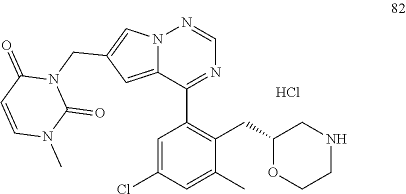 [SMILES: Cc1cc(Cl)cc(-c2ncnn3cc(Cn4c(=O)ccn(C)c4=O)cc23)c1C[C@@H]1CNCCO1 ; Cl]

### Step 1.

### Synthesis of (R)-1-((R)-2-(2-bromo-4-chloro-6-methylbenzyl)morpholino)-2-hydroxy-2-phenylethan-1-one (82a) and (R)-1-((S)-2-(2-bromo-4-chloro-6-methylbenzyl)morpholino)-2-hydroxy-2-phenylethan-1-one (82a′)

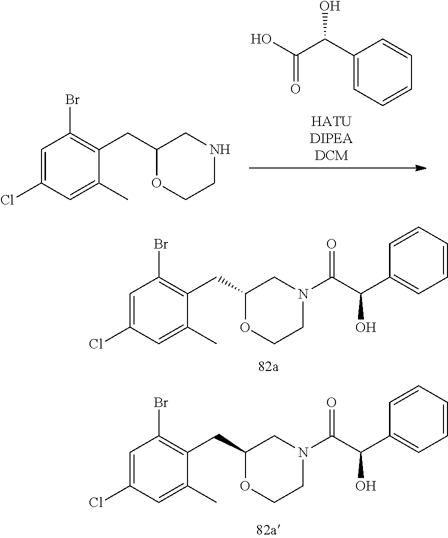 [SMILES: Cc1cc(Cl)cc(Br)c1CC1CNCCO1 ; O=C(O)[C@H](O)c1ccccc1 ; Cc1cc(Cl)cc(Br)c1C[C@@H]1CN(C(=O)[C@H](O)c2ccccc2)CCO1 ; Cc1cc(Cl)cc(Br)c1C[C@H]1CN(C(=O)[C@H](O)c2ccccc2)CCO1]

To the solution of (rac)-2-(2-bromo-4-chloro-6-methylbenzyl)morpholine (the synthesis of this compound was described in J. Med. Chem. 2020, 63, 5398-5420)(1.00 g; 3.28 mmol) in anhydrous DCM (30 mL) (R)-2-hydroxy-2-phenylacetic acid (98%; 0.46 mL; 3.94 mmol) N,N-Diisopropylethylamine (DIPEA) (1.69 mL; 9.84 mmol), HATU (0-(7-Azabenzotriazol-1-yl)-N,N,N,N-tetramethyl uronium hexafluorophosphate) (1.50 g, 3.940 mmol) were added and the reaction mixture was then stirred at room temperature for 1 hour. The reaction progress was monitored by LC-MS. When analysis indicated completion of the reaction, the reaction mixture was washed with 1 M HCl (20 mL), 1 M NaOH (20 mL), brine (20 mL) and then dried over anhydrous Na₂SO₄, filtered and concentrated in vacuo. The crude was then purified by flash column chromatography on silica (hexane/AcOEt, 100:0 to 0:100, v/v, 15 min, 25 mL/min). Compounds 82a and 82a′ were obtained as a mixture of diastereoisomers in 78% yield (1.12 g; 2.55 mmol). Then the diastereoisomers were separated using a chiral preparative HPLC method (LuxR 5 mm Cellulose-4 column, 250×21.2 mm) with propan-2-ol and n-hexane (gradient: 10-90%) as an eluent and UV detection. The retention time of 82a′ in preparative HPLC was in the range of 21.3 to 23.7 min. The retention time of 82a in preparative HPLC was in the range of 28.8 to 35.5 min. The diastereomeric excess for both diastereomers was determined with the use of the chiral analytical HPLC method with UV detection with (LuxR 5 mm Cellulose-4 column, 150×4.6 mm) column using 10-90% propan-2-ol in n-hexane as eluent for 40 min. Compound 82a′ was obtained as a colorless oil in 50.8% yield (0.573 g; 1.31 mmol) with a retention time of 16.2 min and compound 82a was obtained in 47.3% yield (0.533 g; 1.21 mmol) with a retention time of 25.1 min, each with diastereomeric excess of approximately 99%.

For 82a: ESI-MS m/z for C₂₀H₂₂BrClNO₃ found 438.0/439.9 [M+H]⁺; Rₜ=1.62 min (Method A)

For 82a′: ESI-MS m/z for C₂₀H₂₂BrClNO₃ found 437.9/439.9 [M+H]⁺; Rₜ=1.66 min (Method A)

### Step 2.

### Synthesis of (R)-2-(2-bromo-4-chloro-6-methylbenzyl)morpholine (82b)

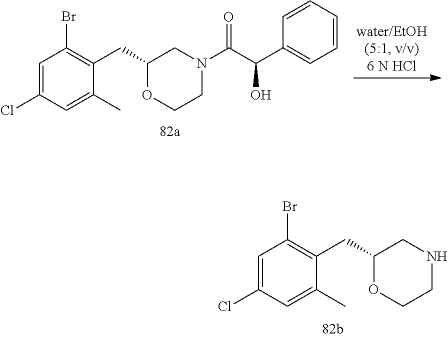 [SMILES: Cc1cc(Cl)cc(Br)c1C[C@@H]1CN(C(=O)[C@H](O)c2ccccc2)CCO1 ; Cc1cc(Cl)cc(Br)c1C[C@@H]1CNCCO1]

To the solution of 82a (1.12 g; 2.55 mmol) in water/EtOH (5:1; v/v; 60 mL) 6 N HCl (100 mL) was added and the resulting mixture was then stirred at 100° C. for 4 hours. The reaction progress was monitored by LC-MS. When analysis indicated completion of the reaction, the reaction mixture was cooled to room temperature and a whole was concentrated in vacuo. The residue was basified with 1 M K₂CO₃ (30 mL) and extracted with AcOEt (3×40 mL). The combined organic solutions were then dried over anhydrous Na₂SO₄, filtered and concentrated in vacuo. Compound 82b was obtained in 99% yield (763 mg; 2.52 mmol).

ESI-MS m/z for C₁₂H₁₆BrClNO found 303.8/305.8 [M+H]⁺; Rₜ=1.03 min (Method A)

### Step 3.

### Synthesis of tert-butyl (R)-2-(2-bromo-4-chloro-6-methylbenzyl)morpholine-4-carboxylate (82c)

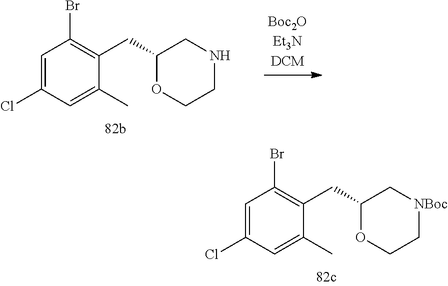 [SMILES: Cc1cc(Cl)cc(Br)c1C[C@@H]1CNCCO1 ; Cc1cc(Cl)cc(Br)c1C[C@@H]1CN(C(=O)OC(C)(C)C)CCO1]

To the solution of 82b (763 mg; 2.52 mmol) and Et₃N (0.53 mL; 3.82 mmol) in DCM (25 mL) Boc₂O (852 mg; 3.82 mmol) was added and this mixture was then stirred at room temperature for 1 hour. The reaction progress was monitored by LC-MS. When analysis indicated completion of the reaction, the solvent was evaporated in vacuo and the residue was then purified by flash column chromatography on silica (hexane/AcOEt, 100:0 to 0:100, v/v, 13 min, 15 mL/min). Compound 82c was obtained in 98% yield (1 g; 2.48 mmol).

ESI-MS m/z for C₁₅H₁₉BrClN₂O₃ found 388.9/390.9 [M+H-tBu+MeCN]⁺; Rₜ=2.02 min (Method A); ¹H NMR (700 MHz, CDCl₃) δ 7.42 (d, J=2.2 Hz, 1H), 7.11 (d, J=2.1 Hz, 1H), 4.02-3.76 (m, 3H), 3.69-3.58 (m, 1H), 3.47-3.34 (m, 1H), 2.99-2.88 (m, 3H), 2.78-2.65 (m, 1H), 2.39 (s, 3H), 1.46 (s, 9H).

### Step 4.

### Synthesis of tert-butyl (R)-2-(4-chloro-2-methyl-6-(4,4,5,5-tetramethyl-1,3,2-dioxaborolan-2-yl)benzyl)morpholine-4-carboxylate (2a)

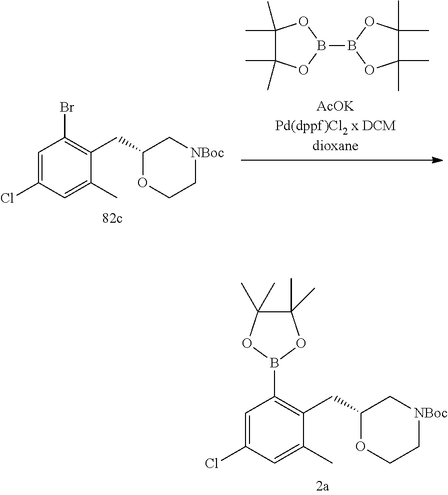

In a Schlenk flask was placed 82c (980 mg; 2.42 mmol), bis(pinacolato)diboron (1.267 g; 4.84 mmol), AcOK (0.45 mL; 7.26 mmol) and Pd(dppf)Cl₂×DCM (202 mg; 0.24 mmol) and dioxane (20 mL). The reaction mixture was intensively flushed with Ar. The mixture was capped and stirred at 90° C. overnight. The reaction progress was monitored by LC-MS. When analysis indicated completion of the reaction, the reaction mixture was concentrated in vacuo. The residue was then purified by flash column chromatography on silica (hexane/AcOEt, 100:0 to 30:70, v/v, 20 min, 15 mL/min). Compound 2a was obtained in 99% yield (1.078 g; 2.39 mmol).

ESI-MS m/z for C₁₈H₂₈BClNO₃ found 352.0/354.0 [M+H-Boc]⁺; Rₜ=2.25 min (Method A)

### Step 5.

### Synthesis of tert-butyl (R)-2-(4-chloro-2-methyl-6-(6-((3-methyl-2,6-dioxo-3,6-dihydropyrimidin-1(2H)-yl)methyl)pyrrolo[2,1-f][1,2,4]triazin-4-yl)benzyl)morpholine-4-carboxylate (8d)

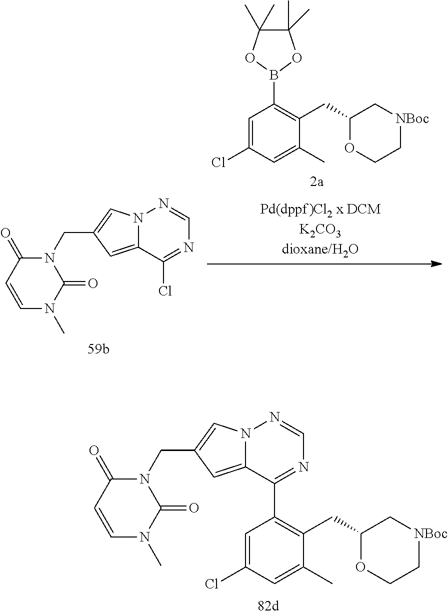 [SMILES: Cc1cc(Cl)cc(B2OC(C)(C)C(C)(C)O2)c1C[C@@H]1CN(C(=O)OC(C)(C)C)CCO1 ; Cn1ccc(=O)n(Cc2cc3c(Cl)ncnn3c2)c1=O ; Cc1cc(Cl)cc(-c2ncnn3cc(Cn4c(=O)ccn(C)c4=O)cc23)c1C[C@@H]1CN(C(=O)OC(C)(C)C)CCO1]

The title compound (82d) was obtained from 59b (258 mg; 0.885 mmol) and from 2a (400 mg; 0.885 mmol) according to the General Procedure IV in 12% yield (200 mg; 0.345 mmol, 30%). The crude product was purified by flash column chromatography on silica (hexane/AcOEt, 100:0 to 0:100, v/v, 30 min, 15 mL/min).

ESI-MS m/z for C₂₉H₃₄ClN₆O₅ found 581.4/583.2 [M+H]⁺; Rₜ=1.60 min (Method A)

### Step 6.

### Synthesis of (R)-3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione hydrochloride (82)

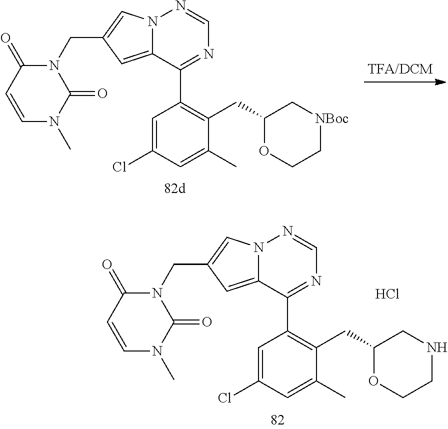 [SMILES: Cc1cc(Cl)cc(-c2ncnn3cc(Cn4c(=O)ccn(C)c4=O)cc23)c1C[C@@H]1CN(C(=O)OC(C)(C)C)CCO1 ; Cc1cc(Cl)cc(-c2ncnn3cc(Cn4c(=O)ccn(C)c4=O)cc23)c1C[C@@H]1CNCCO1 ; Cl]

The title compound (82) was obtained as a single enantiomer of the compound 60 (99% ee) as a hydrochloride salt from 82d (200 mg; 0.103 mmol, 30%) according to the General Procedure IIIb in 81% yield (43 mg; 0.083 mmol). The crude product was purified by preparative reversed-phase column chromatography (C-18, water+0.3‰ HCl (36%)/MeCN, 98:2 to 30:70, 30 min, 20 mL/min, Rₜ=15.25 min).

ESI-MS m/z for C₂₄H₂₆ClN₆O₃ found 481.3/483.1 [M+H]⁺; Rₜ=1.00 min (Method A); ¹H NMR (700 MHz, Methanol-d₄) δ 8.61 (s, 1H), 8.33 (d, J=1.4 Hz, 1H), 7.59-7.53 (m, 2H), 7.42 (d, J=2.3 Hz, 1H), 6.97 (d, J=1.4 Hz, 1H), 5.73 (d, J=7.8 Hz, 1H), 5.23 (s, 2H), 3.77-3.71 (m, 2H), 3.52-3.45 (m, 1H), 3.37 (s, 3H), 3.25-3.16 (m, 1H), 3.13-3.06 (m, 1H), 2.97-2.87 (m, 3H), 2.77-2.71 (m, 1H), 2.49 (s, 3H).

### Example 83

### Synthesis of (R)-3-((4-(5-chloro-3-methyl-2-(morpholin-2-ylmethyl)phenyl)pyrrolo[2,1-f][1,2,4]triazin-6-yl)methyl)-1-(2,2,2-trifluoroethyl)pyrimidine-2,4(1H,3H)-dione hydrochloride (83)

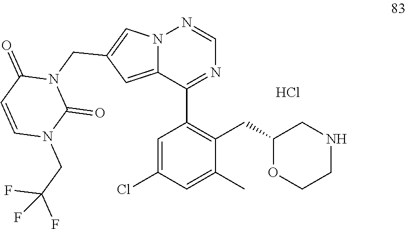 [SMILES: Cc1cc(Cl)cc(-c2ncnn3cc(Cn4c(=O)ccn(CC(F)(F)F)c4=O)cc23)c1C[C@@H]1CNCCO1 ; Cl]

### Step 1.

### Synthesis of tert-butyl (R)-2-(4-chloro-2-(6-((2,6-dioxo-3-(2,2,2-trifluoroethyl)-3,6-dihydropyrimidin-1(2H)-yl)methyl)pyrrolo[2,1-f][1,2,4]triazin-4-yl)-6-methylbenzyl)morpholine-4-carboxylate (83a)

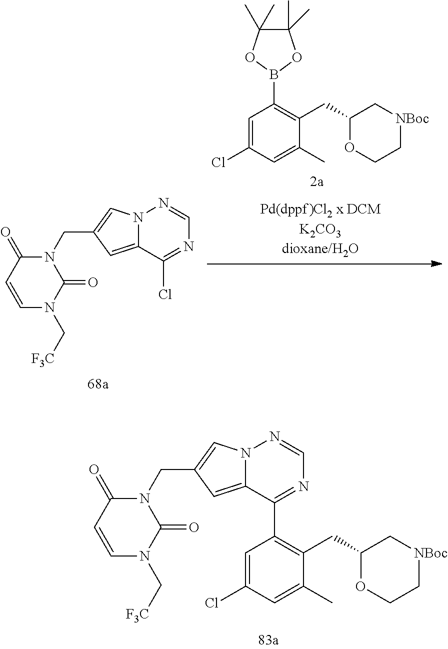 [SMILES: Cc1cc(Cl)cc(B2OC(C)(C)C(C)(C)O2)c1C[C@@H]1CN(C(=O)OC(C)(C)C)CCO1 ; O=c1ccn(CC(F)(F)F)c(=O)n1Cc1cc2c(Cl)ncnn2c1 ; Cc1cc(Cl)cc(-c2ncnn3cc(Cn4c(=O)ccn(CC(F)(F)F)c4=O)cc23)c1C[C@@H]1CN(C(=O)OC(C)(C)C)CCO1]

The title compound (83a) was obtained from 68a (79.5 mg; 0.221 mmol) and from 2a (100 mg; 0.221 mmol) according to the General Procedure IV in 66% yield (95 mg; 0.146 mmol). The crude product was purified by flash column chromatography on silica (hexane/AcOEt, 100:0 to 0:100, v/v, 45 min, 15 mL/min).

ESI-MS m/z for C₃₀H₃₃ClF₃N₆O₅ found 649.4/651.4 [M+H]⁺; Rₜ=1.81 min (Method A)

### Step 2.

### Synthesis of (R)-3-((4-(5-chloro-3-methyl-2-(morpholin-2-ylmethyl)phenyl)pyrrolo[2,1-f][1,2,4]triazin-6-yl)methyl)-1-(2,2,2-trifluoroethyl)pyrimidine-2,4(1H,3H)-dione hydrochloride (83)

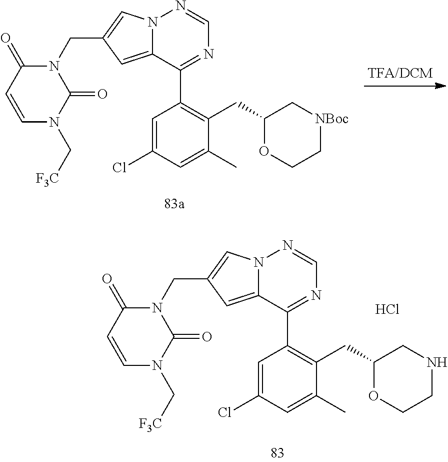 [SMILES: Cc1cc(Cl)cc(-c2ncnn3cc(Cn4c(=O)ccn(CC(F)(F)F)c4=O)cc23)c1C[C@@H]1CN(C(=O)OC(C)(C)C)CCO1 ; Cc1cc(Cl)cc(-c2ncnn3cc(Cn4c(=O)ccn(CC(F)(F)F)c4=O)cc23)c1C[C@@H]1CNCCO1 ; Cl]

The title compound (88) was obtained as a single enantiomer of the compound 68 (99% ee) as a hydrochloride salt from 83a (92 mg; 0.142 mmol) according to the General Procedure IIIb in 11% yield (9.5 mg; 0.016 mmol). The crude product was purified by preparative reversed-phase column chromatography (C-18, water+0.3‰ HCl (36%)/MeCN, 99:1 to 20:80, 30 min, 20 mL/min, Rₜ=16.34 min). The impure product was purified on a TLC plate (DCM/MeOH/AcOH, 90:10:2, v/v/v) and then the product was washed out from silica gel with MeOH and MeCN. The silica gel with solvents was sonicated and filtered (5×). the combined filtrates were concentrated in vacuo and then purified by preparative reversed-phase column chromatography (C-18, water+0.3‰ HCl (36%)/MeCN, 98:2 to 30:70, 30 min, 20 mL/min, Rₜ=17.10 min).

ESI-MS m/z for C₂₅H₂₅ClF₃N₆O₃ found 549.4/551.4 [M+H]⁺; Rₜ=1.14 min (Method A); ¹H NMR (700 MHz, D₂O) δ 8.41 (s, 1H), 8.03 (s, 1H), 7.60 (d, J=7.9 Hz, 1H), 7.45 (s, 1H), 7.27 (s, 1H), 6.58 (s, 1H), 5.90 (d, J=7.9 Hz, 1H), 5.16 (s, 2H), 4.56-4.52 (m, 2H), 3.56-3.48 (m, 2H), 3.30-3.25 (m, 1H), 3.07-3.02 (m, 2H), 2.95-2.91 (m, 1H), 2.88-2.78 (m, 2H), 2.64-2.60 (m, 1H), 2.35 (s, 3H).

### Example 84

### Synthesis of 3-((4-(5-chloro-3-methyl-2-(1,2,3,6-tetrahydropyridin-4-yl)phenyl)pyrrolo[2,1-f][1,2,4]triazin-6-yl)methyl)-6,6-dimethyl-3-azabicyclo[3.1.0]hexane-2,4-dione hydrochloride (84)

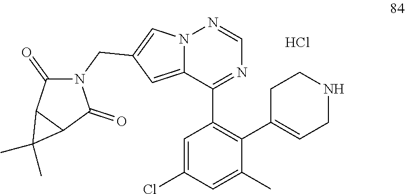 [SMILES: Cc1cc(Cl)cc(-c2ncnn3cc(CN4C(=O)C5C(C4=O)C5(C)C)cc23)c1C1=CCNCC1 ; Cl]

### Step 1.

### Synthesis of tert-butyl 4-(2-bromo-4-chloro-6-methylphenyl)-3,6-dihydropyridine-1(2H)-carboxylate (84a)

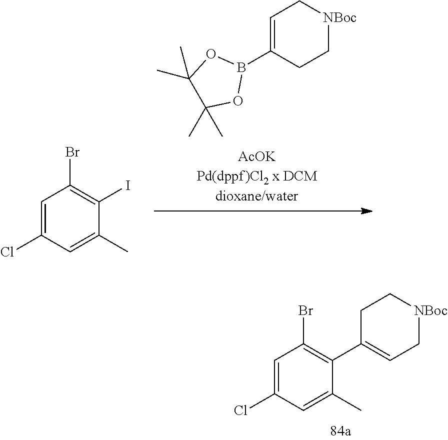

In a Schlenk flask was placed 1-bromo-5-chloro-2-iodo-3-methylbenzene (200 mg; 0.604 mmol), tert-butyl 4-(4,4,5,5-tetramethyl-1,3,2-dioxaborolan-2-yl)-3,6-dihydropyridine-1(2H)-carboxylate (191 mg; 0.604 mmol), AcOK (0.07 mL; 1.210 mmol) and Pd(dppf)Cl₂×DCM (50 mg; 0.060 mmol), dioxane (4 mL) and water (0.8 mL). The reaction mixture was intensively flushed with Ar. The mixture was capped and stirred at 90° C. for 5 hours. The reaction progress was monitored by LC-MS. When analysis indicated completion of the reaction, the reaction mixture was concentrated in vacuo. The residue was then purified by flash column chromatography on silica (hexane/AcOEt, 100:0 to 0:100, v/v, 15 min, 13 mL/min). Compound 84a was obtained in 57% yield (133 mg; 0.345 mmol).

ESI-MS m/z for C₁₅H₁₇BrClN₂O₂ found 370.9/372.9 [M+H-tBu+MeCN]⁺; Rₜ=2.12 min (Method A)

### Step 2.

### Synthesis of tert-butyl 4-(4-chloro-2-methyl-6-(4,4,5,5-tetramethyl-1,3,2-dioxaborolan-2-yl)phenyl)-3,6-dihydropyridine-1(2H)-carboxylate (84b)

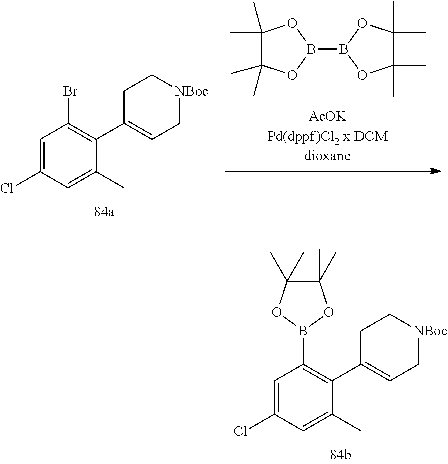

In a Schlenk flask was placed 84a (133 mg; 0.345 mmol), bis(pinacolato)diboron (180 mg; 0.688 mmol), AcOK (0.065 mL; 1.030 mmol) and Pd(dppf)Cl₂×DCM (29 mg; 0.034 mmol) and dioxane (3 mL). The reaction mixture was intensively flushed with Ar. The mixture was capped and stirred at 90° C. overnight. The reaction progress was monitored by LC-MS. When analysis indicated completion of the reaction, the reaction mixture was concentrated in vacuo. The residue was then purified by flash column chromatography on silica (hexane/AcOEt, 100:0 to 0:100, v/v, 13 min, 13 mL/min). Compound 84b was obtained in 94% yield (140 mg; 0.323 mmol)

ESI-MS m/z for C₂₁H₂₉BClN₂O₄ found 419.0/421.0 [M+H-tBu+MeCN]⁺; Rₜ=2.29 min (Method A)

### Step 3.

### Synthesis of tert-butyl 4-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenyl)-3,6-dihydropyridine-1(2H)-carboxylate (84c)

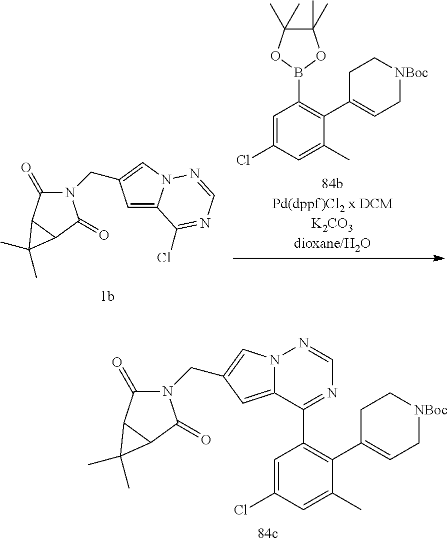 [SMILES: Cc1cc(Cl)cc(B2OC(C)(C)C(C)(C)O2)c1C1=CCN(C(=O)OC(C)(C)C)CC1 ; CC1(C)C2C(=O)N(Cc3cc4c(Cl)ncnn4c3)C(=O)C21 ; Cc1cc(Cl)cc(-c2ncnn3cc(CN4C(=O)C5C(C4=O)C5(C)C)cc23)c1C1=CCN(C(=O)OC(C)(C)C)CC1]

The title compound (84c) was obtained from 1b (98 mg; 0.323 mmol) and from 84b (140 mg; 0.323) according to the General Procedure IV in 51% yield (95 mg; 0.165 mmol). The crude product was purified by flash column chromatography on silica (hexane/AcOEt, 100:0 to 0:100, v/v, 30 min, 15 mL/min).

ESI-MS m/z for C₃₁H₃₅ClN₅O₄ found 576.1/578.1 [M+H]⁻; Rₜ=1.91 min (Method A)

### Step 4.

### Synthesis of 3-((4-(5-chloro-3-methyl-2-(1,2,3,6-tetrahydropyridin-4-yl)phenyl)pyrrolo[2,1-f][1,2,4]triazin-6-yl)methyl)-6,6-dimethyl-3-azabicyclo[3.1.0]hexane-2,4-dione hydrochloride (84)

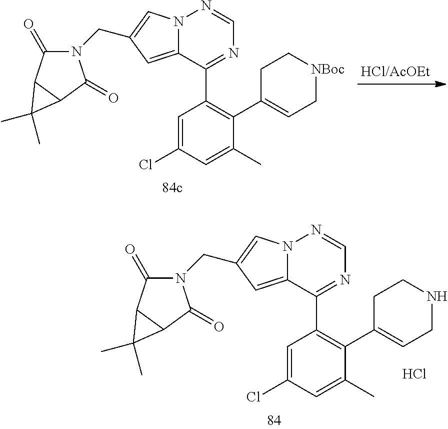 [SMILES: Cc1cc(Cl)cc(-c2ncnn3cc(CN4C(=O)C5C(C4=O)C5(C)C)cc23)c1C1=CCN(C(=O)OC(C)(C)C)CC1 ; Cc1cc(Cl)cc(-c2ncnn3cc(CN4C(=O)C5C(C4=O)C5(C)C)cc23)c1C1=CCNCC1 ; Cl]

The title compound (84) was obtained as a hydrochloride salt from 84c (95 mg; 0.165 mmol) according to the General Procedure IIIa in 13% yield (11 mg; 0.021 mmol). The crude product was purified by preparative reversed-phase column chromatography (C-18, water+0.3‰ TFA/MeCN, 99:1 to 20:80, 30 min, 20 mL/min, Rₜ=15.75 min).

ESI-MS m/z for C₂₆H₂₇ClN₅O₂ found 476.4/478.4 [M+H]⁺; Rₜ=1.18 min (Method A); ¹H NMR (700 MHz, Methanol-d₄) δ 8.51 (s, 1H), 8.08 (d, J=1.4 Hz, 1H), 7.54 (d, J=2.1 Hz, 1H), 7.44 (d, J=2.2 Hz, 1H), 6.75 (d, J=1.5 Hz, 1H), 5.46-5.38 (m, 1H), 4.66 (s, 2H), 3.72-3.59 (m, 1H), 3.48-3.33 (m, 2H), 3.11-2.96 (m, 1H), 2.63-2.51 (m, 2H), 2.46 (s, 2H), 2.40 (s, 3H), 1.23 (s, 3H), 1.05 (s, 3H).

### Example 85

### Synthesis of 3-((4-(5-chloro-2-(piperazin-1-yl)phenyl)pyrrolo[2,1-f][1,2,4]triazin-6-yl)methyl)-6,6-dimethyl-3-azabicyclo[3.1.0]hexane-2,4-dione hydrochloride (85)

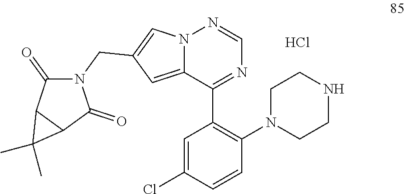 [SMILES: CC1(C)C2C(=O)N(Cc3cc4c(-c5cc(Cl)ccc5N5CCNCC5)ncnn4c3)C(=O)C21 ; Cl]

The title compound (85) was obtained as a hydrochloride salt in 13% overall yield in a similar way to Example 2 with the exception that, in the first step of the synthesis, tert-butyl 4-(2-bromo-4-chlorophenyl)piperazine-1-carboxylate (the synthesis of this compound was described below) was used instead of tert-butyl (R)-2-(2-bromo-4-chloro-6-methylbenzyl)morpholine-4-carboxylate and in the last step of the synthesis, the crude product was purified by preparative reversed-phase column chromatography (column: Cosmosil Cholester 20×250 mm, water+0.3‰ HCl (36%)/MeCN, 99:1 to 40:60, 30 min, 20 mL/min, Rₜ=18.31 min).

ESI-MS m/z for C₂₄H₂₆ClN₆O₂ found 465.4/467.4 [M+H]⁺; Rₜ=1.10 min (Method A); ¹H NMR (700 MHz, Methanol-d₄) δ 8.56 (s, 1H), 8.09 (d, J=1.2 Hz, 1H), 7.63-7.60 (m, 1H), 7.57 (d, J=2.5 Hz, 1H), 7.36 (d, J=8.7 Hz, 1H), 6.93 (s, 1H), 4.69 (s, 2H), 3.19-3.13 (m, 4H), 3.02-2.95 (m, 4H), 2.48 (s, 2H), 1.24 (s, 3H), 1.09 (s, 3H).

### Example 86

### Synthesis of 3-((4-(2-((S)-3-aminopyrrolidin-1-yl)-5-chlorophenyl)pyrrolo[2,1-f][1,2,4]triazin-6-yl)methyl)-6,6-dimethyl-3-azabicyclo[3.1.0]hexane-2,4-dione hydrochloride (86)

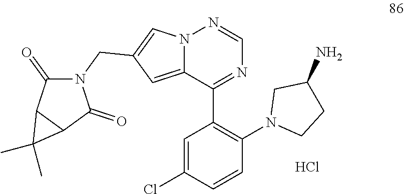 [SMILES: CC1(C)C2C(=O)N(Cc3cc4c(-c5cc(Cl)ccc5N5CC[C@H](N)C5)ncnn4c3)C(=O)C21 ; Cl]

The title compound (86) was obtained as a hydrochloride salt in 10% overall yield in a similar way to Example 2 with the exception that, in the first step of the synthesis, tert-butyl (S)-(1-(2-bromo-4-chlorophenyl)pyrrolidin-3-yl)carbamate (86b) (the synthesis of this compound was described below) was used instead of tert-butyl (R)-2-(2-bromo-4-chloro-6-methylbenzyl)morpholine-4-carboxylate and tris(dibenzylideneacetone)dipalladium(0) (Pd₂(dba)₃) was used in this step and in the last step of the synthesis, the crude product was purified by preparative reversed-phase column chromatography (column: Cosmosil Cholester 20×250 mm, water+0.3‰ HCl (36%)/MeCN, 99:1 to 40:60, 30 min, 20 mL/min, Rₜ=18.00 min).

ESI-MS m/z for C₂₄H₂₆ClN₆O₂ found 465.4/467.4 [M+H]⁺; Rₜ=1.08 min (Method A); ¹H NMR (700 MHz, Methanol-d₄+D₂O) δ 8.49 (s, 1H), 8.08 (d, J=1.5 Hz, 1H), 7.48-7.44 (m, 1H), 7.42 (d, J=2.5 Hz, 1H), 7.06 (d, J=8.9 Hz, 1H), 6.90 (d, J=1.4 Hz, 1H), 4.65 (s, 2H), 3.82-3.74 (m, 1H), 3.36-3.30 (m, 1H), 3.26-3.19 (m, 1H), 3.11-3.02 (m, 2H), 2.44 (s, 2H), 2.29-2.19 (m, 1H), 1.98-1.88 (m, 1H), 1.21 (s, 3H), 1.04 (s, 3H).

### Synthesis of tert-butyl (S)-(1-(2-bromo-4-chlorophenyl)pyrrolidin-3-yl)carbamate (86b)

### Step 1.

### Synthesis of (S)-1-(2-bromo-4-chlorophenyl)pyrrolidin-3-amine (86a)

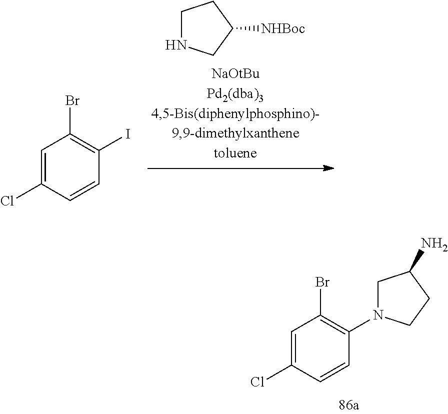 [SMILES: CC(C)(C)OC(=O)N[C@H]1CCNC1 ; Clc1ccc(I)c(Br)c1 ; N[C@H]1CCN(c2ccc(Cl)cc2Br)C1]

To the solution of 2-bromo-4-chloro-1-iodobenzene (300 mg; 0.945 mmol), tert-butyl (S)-pyrrolidin-3-ylcarbamate (180 mg; 0.945 mmol) and 4,5-Bis(diphenylphosphino)-9,9-dimethylxanthene (55 mg; 0.094 mmol) in toluene (5 mL) NaOtBu (185 mg; 1.890 mmol) was added and the suspension was intensively flushed with Ar. Then to this suspension tris(dibenzylideneacetone)dipalladium(0) (Pd₂(dba)₃; 91 mg; 0.094 mmol) was added and a whole was stirred at 100° C. overnight in a sealed tube. The reaction progress was monitored by LC-MS. The reaction mixture was concentrated in vacuo and the residue was used to the next step without any additional purification.

ESI-MS m/z for C₁₀H₁₃BrClN₂ found 274.9/276.9 [M+H]⁺; Rₜ=0.94 min (Method A)

### Step 2.

### Synthesis of tert-butyl (S)-(1-(2-bromo-4-chlorophenyl)pyrrolidin-3-yl)carbamate (86b)

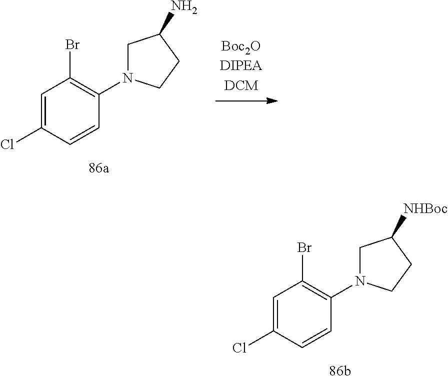 [SMILES: N[C@H]1CCN(c2ccc(Cl)cc2Br)C1 ; CC(C)(C)OC(=O)N[C@H]1CCN(c2ccc(Cl)cc2Br)C1]

To the solution of 86a (the crude product) in DCM (4 mL) Boc₂O (240 mg; 1.100 mmol) and DIPEA (0.05 mL; 0.291 mmol) were added and this mixture was then stirred at room temperature overnight. The reaction progress was monitored by LC-MS. When analysis indicated completion of the reaction, the solvent was evaporated in vacuo and the residue was then purified by flash column chromatography on silica (hexane/AcOEt, 100:0 to 80:20, v/v, 30 min, 13 mL/min). Compound 86b was obtained in 37% yield (per two steps)(130 mg; 0.347 mmol).

ESI-MS m/z for C₁₅H₂₁BrClN₂O₂ found 374.9/376.9 [M+H]⁺; Rₜ=1.84 min (Method A)

### Example 87

### Synthesis of 3-((4-(2-((R)-3-aminopyrrolidin-1-yl)-5-chlorophenyl)pyrrolo[2,1-f][1,2,4]triazin-6-yl)methyl)-6,6-dimethyl-3-azabicyclo[3.1.0]hexane-2,4-dione hydrochloride (87)

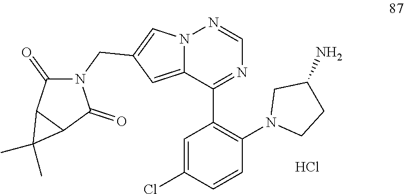 [SMILES: CC1(C)C2C(=O)N(Cc3cc4c(-c5cc(Cl)ccc5N5CC[C@@H](N)C5)ncnn4c3)C(=O)C21 ; Cl]

The title compound (87) was obtained as a hydrochloride salt in 9% overall yield in a similar way to Example 2 with the exception that, in the first step of the synthesis, tert-butyl (R)-(1-(2-bromo-4-chlorophenyl)pyrrolidin-3-yl)carbamate (the synthesis of this compound was the same as for 86b, starting from tert-butyl (R)-pyrrolidin-3-ylcarbamate instead of tert-butyl (S)-pyrrolidin-3-ylcarbamate) was used instead of tert-butyl (R)-2-(2-bromo-4-chloro-6-methylbenzyl)morpholine-4-carboxylate and in the last step of the synthesis, the crude product was purified by preparative reversed-phase column chromatography (column: Cosmosil Cholester 20×250 mm, water+0.3‰ HCl (36%)/MeCN, 99:1 to 40:60, 30 min, 20 mL/min, Rₜ=17.80 min).

ESI-MS m/z for C₂₄H₂₆ClN₆O₂ found 465.4/467.0 [M+H]⁺; Rₜ=1.11 min (Method A); ¹H NMR (700 MHz, Methanol-d₄+D₂O) δ 8.53 (s, 1H), 8.11 (d, J=1.4 Hz, 1H), 7.50-7.45 (m, 1H), 7.44 (d, J=2.5 Hz, 1H), 7.06 (d, J=8.9 Hz, 1H), 6.93 (d, J=1.4 Hz, 1H), 4.66 (s, 2H), 3.84-3.74 (m, 1H), 3.38-3.31 (m, 1H), 3.27-3.20 (m, 1H), 3.16-3.01 (m, 2H), 2.45 (s, 2H), 2.30-2.17 (m, 1H), 1.99-1.89 (m, 1H), 1.21 (s, 3H), 1.04 (s, 3H).

### Example 88

### Synthesis of 3-((4-(5-chloro-3-methyl-2-(1,2,3,6-tetrahydropyridin-4-yl)phenyl)pyrrolo[2,1-f][1,2,4]triazin-6-yl)methyl)-1-methylpyrimidine-2,4(1H,3H)-dione hydrochloride (88)

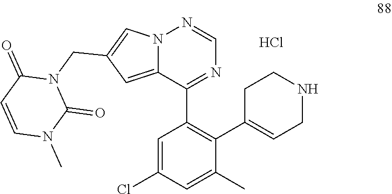 [SMILES: Cc1cc(Cl)cc(-c2ncnn3cc(Cn4c(=O)ccn(C)c4=O)cc23)c1C1=CCNCC1 ; Cl]

The title compound (88) was obtained as a hydrochloride salt in 15% overall yield in a similar way to Example 84 with the exception that, in the third step of the synthesis, the compound 59b was used instead of the compound 1b, and in the last step of the synthesis, the General Procedure IIIb was used instead of the General Procedure IIIa and the crude product was purified by preparative reversed-phase column chromatography (C-18, water+0.3‰ HCl (36%)/MeCN, 98:2 to 30:70, 30 min, 20 mL/min, Rₜ=15.80 min).

ESI-MS m/z for C₂₄H₂₄ClN₆O₂ found 463.4/465.4 [M+H]⁺; Rₜ=1.01 min (Method A); ¹H NMR (700 MHz, Methanol-d₄) δ 8.61 (s, 1H), 8.38 (d, J=1.4 Hz, 1H), 7.64-7.60 (m, 1H), 7.57 (d, J=7.8 Hz, 1H), 7.54-7.46 (m, 1H), 7.10 (d, J=1.5 Hz, 1H), 5.74 (d, J=7.9 Hz, 1H), 5.59-5.53 (m, 1H), 5.23 (s, 2H), 3.76-3.61 (m, 1H), 3.53-3.41 (m, 1H), 3.38 (s, 3H), 3.36-3.32 (m, 1H), 3.09-2.97 (m, 1H), 2.61-2.51 (m, 1H), 2.46-2.37 (m, 4H).

### Example 89

### Synthesis of (R)-3-((4-(5-chloro-3-methyl-2-(morpholin-2-ylmethyl)phenyl)pyrrolo[2,1-f][1,2,4]triazin-6-yl)methyl)-1-isopropylpyrimidine-2,4(1H,3H)-dione hydrochloride (89)

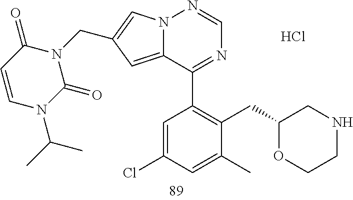 [SMILES: Cc1cc(Cl)cc(-c2ncnn3cc(Cn4c(=O)ccn(C(C)C)c4=O)cc23)c1C[C@@H]1CNCCO1 ; Cl]

### Step 1.

### Synthesis of tert-butyl 2,4-dioxo-3,4-dihydropyrimidine-1(2H)-carboxylate (89a)

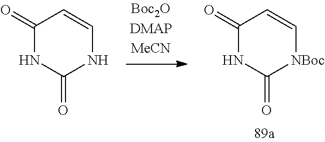 [SMILES: O=c1cc[nH]c(=O)[nH]1 ; CC(C)(C)OC(=O)n1ccc(=O)[nH]c1=O]

To the solution of pyrimidine-2,4(1H,3H)-dione (3 g; 26.00 mmol) in MeCN (150 mL) Boc₂O (5.79 g; 26.00 mmol) and DMAP (33 mg; 0.26 mmol) were added and this mixture was then stirred at room temperature for 3 hours. The reaction progress was monitored by LC-MS. When analysis indicated completion of the reaction, the solvent was evaporated in vacuo and the residue was then purified by flash column chromatography on silica (hexane/AcOEt, 100:0 to 0:100, v/v, 25 min, 35 mL/min). Compound 89a was obtained in 87% yield (4.79 g; 22.58 mmol).

ESI-MS m/z for C₄H₅N₂O₂ found 113.2 [M+H]⁺; Rₜ=0.67 min (Method A); ¹H NMR (700 MHz, CDCl₃) δ 8.38 (s, 1H), 7.88 (d, J=8.5 Hz, 1H), 5.83-5.75 (m, 1H), 1.61 (s, 9H).

### Step 2.

### Synthesis of tert-butyl 3-((4-chloropyrrolo[2,1-f][1,2,4]triazin-6-yl)methyl)-2,4-dioxo-3,4-dihydropyrimidine-1(2H)-carboxylate (89b)

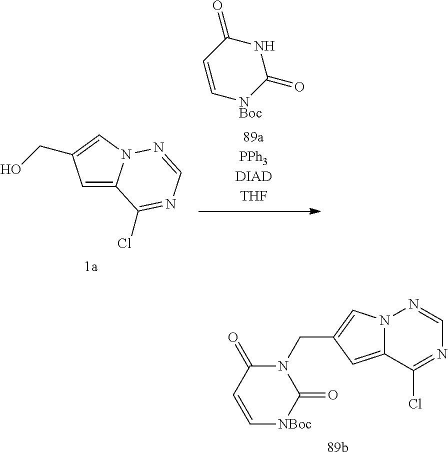 [SMILES: CC(C)(C)OC(=O)n1ccc(=O)[nH]c1=O ; OCc1cc2c(Cl)ncnn2c1 ; CC(C)(C)OC(=O)n1ccc(=O)n(Cc2cc3c(Cl)ncnn3c2)c1=O]

To a cooled to −10° C. solution of 1a (751 mg; 4.09 mmol), 89a (868 mg; 4.09 mmol) and PPh₃ (1.287 g; 4.91 mmol) in dry THE (15 mL) DIAD (1.07 mL; 5.32 mmol) was added. The resulting mixture was stirred at room temperature for 2 hours. Then the reaction mixture was concentrated in vacuo and the residue was purified by flash column chromatography on silica (hexane/AcOEt, 100:0 to 0:100, v/v, 20 min, 25 mL/min). Compound 89b was obtained in 99% yield (1.527 g; 4.05 mmol).

ESI-MS m/z for C₁₂H₁₁ClN₅O₂ found 378.0/379.9 [M+H]⁺; Rₜ=1.36 min (Method A); ¹H NMR (700 MHz, CDCl₃) δ 8.17 (s, 1H), 8.01-7.97 (m, 1H), 7.80 (d, J=8.4 Hz, 1H), 7.09-7.05 (m, 1H), 5.85 (d, J=8.4 Hz, 1H), 5.20 (s, 2H), 1.61 (s, 9H).

### Step 3.

### Synthesis of tert-butyl (R)-2-(4-chloro-2-(6-((2,6-dioxo-3,6-dihydropyrimidin-1(2H)-yl)methyl)pyrrolo[2,1-f][1,2,4]triazin-4-yl)-6-methylbenzyl)morpholine-4-carboxylate (89c)

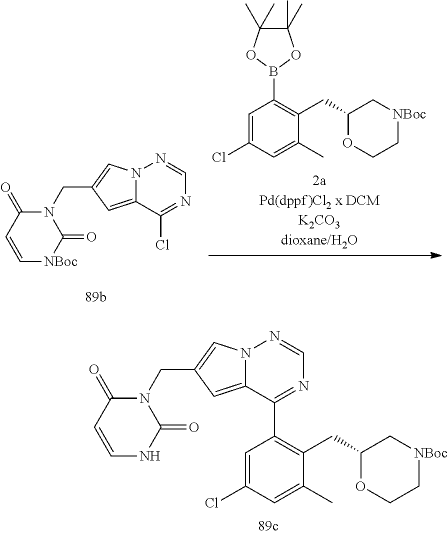 [SMILES: Cc1cc(Cl)cc(B2OC(C)(C)C(C)(C)O2)c1C[C@@H]1CN(C(=O)OC(C)(C)C)CCO1 ; CC(C)(C)OC(=O)n1ccc(=O)n(Cc2cc3c(Cl)ncnn3c2)c1=O ; Cc1cc(Cl)cc(-c2ncnn3cc(Cn4c(=O)cc[nH]c4=O)cc23)c1C[C@@H]1CN(C(=O)OC(C)(C)C)CCO1]

The title compound (89c) was obtained from 89b (83.5 mg; 0.221 mmol) and from 2a (obtained according to the procedure described in the example 82)(100 mg; 0.221 mmol) according to the General Procedure IV in 58% yield (73 mg; 0.129 mmol). The crude product was purified by flash column chromatography on silica (hexane/AcOEt, 100:0 to 0:100, v/v, 30 min, 13 mL/min).

ESI-MS m/z for C₂₈H₃₂ClN₆O₅ found 567.0/569.2 [M+H]⁺; Rₜ=1.50 min (Method A)

### Step 4.

### Synthesis of tert-butyl (R)-2-(4-chloro-2-(6-((3-isopropyl-2,6-dioxo-3,6-dihydropyrimidin-1(2H)-yl)methyl)pyrrolo[2,1-f][1,2,4]triazin-4-yl)-6-methylbenzyl)morpholine-4-carboxylate (89d)

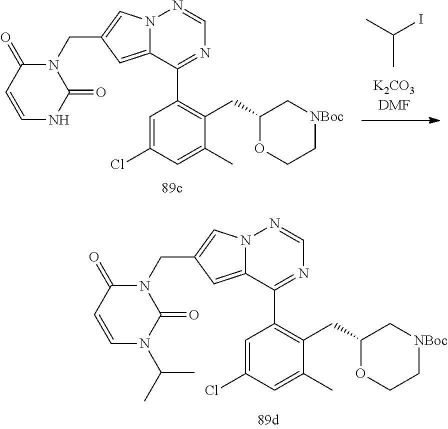

To the solution of 89c (70 mg; 0.123 mmol) in dry DMF (2 mL) K₂CO₃ (35 mg; 0.246 mmol) and 2-iodopropane (stabilized with copper; 21.3 mg; 0.123 mmol) were added. The resulting mixture was stirred at room temperature overnight. The reaction progress was monitored by LC-MS. When analysis indicated completion of the reaction, the reaction mixture was diluted with AcOEt (15 mL) and washed with water (10 mL). The organic layer was then dried over anhydrous Na₂SO₄, filtered and concentrated in vacuo and the residue was purified by flash column chromatography on silica (hexane/AcOEt, 100:0 to 0:100, v/v, 20 min, 15 mL/min). Compound 89d was obtained in 71% yield (53 mg; 0.087 mmol).

ESI-MS m/z for C₃₁H₃₈ClN₆O₅ found 609.4/611.2 [M+H]⁺; Rₜ=1.80 min (Method A)

### Step 5.

### Synthesis of (R)-3-((4-(5-chloro-3-methyl-2-(morpholin-2-ylmethyl)phenyl)pyrrolo[2,1-f][1,2,4]triazin-6-yl)methyl)-1-isopropylpyrimidine-2,4(1H,3H)-dione hydrochloride (89)

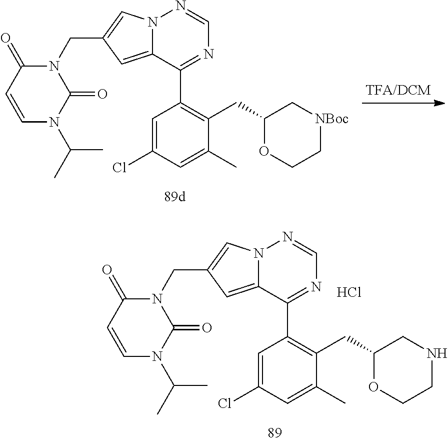 [SMILES: Cc1cc(Cl)cc(-c2ncnn3cc(Cn4c(=O)ccn(C(C)C)c4=O)cc23)c1C[C@@H]1CN(C(=O)OC(C)(C)C)CCO1 ; Cc1cc(Cl)cc(-c2ncnn3cc(Cn4c(=O)ccn(C(C)C)c4=O)cc23)c1C[C@@H]1CNCCO1 ; Cl]

The title compound (89) was obtained as a single enantiomer (99% ee) as a hydrochloride salt from 89d (50 mg; 0.082 mmol) according to the General Procedure IIIb in 56% yield (25 mg; 0.046 mmol). The crude product was purified by preparative reversed-phase column chromatography (C-18, water+0.3‰ HCl (36%)/MeCN, 98:2 to 25:75, 30 min, 15 mL/min, Rₜ=17.00 min).

ESI-MS m/z for C₂₆H₃₀ClN₆O₃ found 509.5/511.5 [M+H]⁺; Rₜ=1.10 min (Method A); ¹H NMR (700 MHz, Methanol-d₄) δ 8.62 (s, 1H), 8.32 (d, J=1.5 Hz, 1H), 7.72 (d, J=8.0 Hz, 1H), 7.58 (d, J=2.2 Hz, 1H), 7.45 (d, J=2.5 Hz, 1H), 6.95 (d, J=1.5 Hz, 1H), 5.83 (d, J=8.0 Hz, 1H), 5.27 (s, 2H), 4.85-4.82 (m, 1H), 3.81-3.75 (m, 2H), 3.55-3.49 (m, 1H), 3.25-3.21 (m, 1H), 3.15-3.11 (m, 1H), 3.02-2.93 (m, 3H), 2.82-2.77 (m, 1H), 2.53 (s, 3H), 1.37 (d, J=6.8 Hz, 6H).

### Example 90

### Synthesis of 3-((4-(5-chloro-2-((3aR,6aR)-hexahydropyrrolo[3,4-b]pyrrol-5(1H)-yl)phenyl)pyrrolo[2,1-f][1,2,4]triazin-6-yl)methyl)-6,6-dimethyl-3-azabicyclo[3.1.0]hexane-2,4-dione hydrochloride (90)

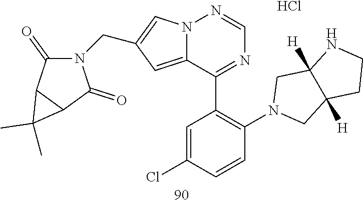 [SMILES: Cl ; CC1(C)C2C(=O)N(Cc3cc4c(-c5cc(Cl)ccc5N5C[C@H]6CCN[C@H]6C5)ncnn4c3)C(=O)C21]

### Step 1.

### Synthesis of tert-butyl (3aR,6aR)-5-(2-bromo-4-chlorophenyl)hexahydropyrrolo[3,4-b]pyrrole-1(2H)-carboxylate (90a)

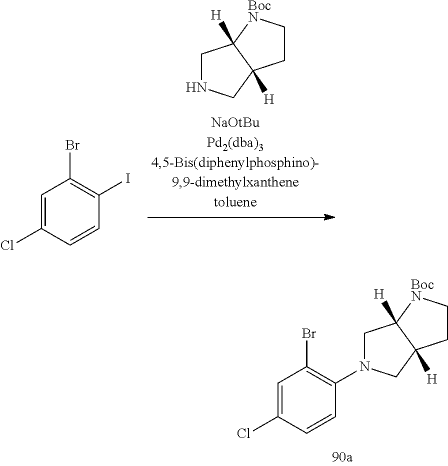 [SMILES: CC(C)(C)OC(=O)N1CC[C@@H]2CNC[C@@H]21 ; Clc1ccc(I)c(Br)c1 ; CC(C)(C)OC(=O)N1CC[C@@H]2CN(c3ccc(Cl)cc3Br)C[C@@H]21]

To the solution of 2-bromo-4-chloro-1-iodobenzene (146 mg; 0.462 mmol), tert-butyl (3aR,6aR)-hexahydropyrrolo[3,4-b]pyrrole-1(2H)-carboxylate (101 mg; 0.462 mmol) and 4,5-Bis(diphenylphosphino)-9,9-dimethylxanthene (27 mg; 0.046 mmol) in toluene (2 mL) NaOtBu (90.5 mg; 0.923 mmol) was added and the suspension was intensively flushed with Ar. Then to this suspension tris(dibenzylideneacetone)dipalladium(0) (Pd₂(dba)₃; 44 mg; 0.046 mmol) was added and the whole was stirred at 100° C. overnight in a sealed tube. The reaction progress was monitored by LC-MS. The reaction mixture was concentrated in vacuo and the residue was purified by flash column chromatography on silica (column 4 g, hexane/AcOEt, 100:0 to 30:70, v/v, 20 min, 10 mL/min). Compound 90a was obtained in 49% yield (90 mg; 0.225 mmol).

ESI-MS m/z for C₁₇H₂₃BrClN₂O₂ found 401.0/403.0 [M+H]⁺; Rₜ=2.03 min (Method A)

### Step 2.

### Synthesis of (2-((3aR,6aR)-1-(tert-butoxycarbonyl)hexahydropyrrolo[3,4-b]pyrrol-5(1H)-yl)-5-chlorophenyl)boronic acid (90b)

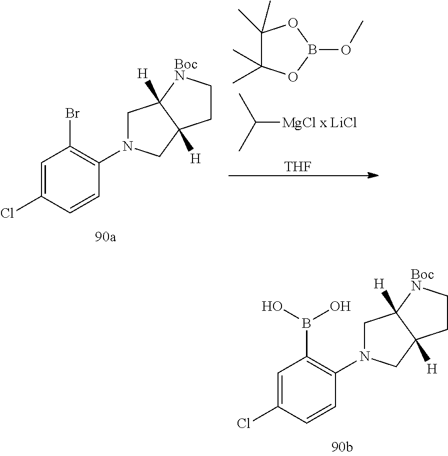

To the cooled to −20° C. solution of isopropyl magnesium chloride lithium chloride complex (1.3 M in THF; 0.46 mL; 0.597 mmol) in dry THF (0.1 mL) a solution of 90a (80 mg; 0.199 mmol) in dry THE (0.1 mL) was added and after stirring in this temperature for 0.5 hour (LC-MS monitoring) 2-isopropoxy-4,4,5,5-tetramethyl-1,3,2-dioxaborolane (84 μL; 0.398 mmol) was added and then the reaction mixture was allowed to warm to room temperature and stirred for 1 hour. The reaction progress was monitored by LC-MS. When analysis indicated completion of the reaction, the reaction mixture was quenched with water and a whole was concentrated in vacuo. To the residue water was added and a whole was extracted with AcOEt (3×). The combined organic layers were dried over anhydrous Na₂SO₄, filtered and concentrated in vacuo. The crude product was used to the next step without any additional purification.

ESI-MS m/z for C₁₇H₂₅BClN₂O₄ found 367.0/369.0 [M+H]⁺; Rₜ=1.46 min (Method A)

### Step 3.

### Synthesis of tert-butyl (3aR,6aR)-5-(4-chloro-2-(6-((6,6-dimethyl-2,4-dioxo-3-azabicyclo[3.1.0]hexan-3-yl)methyl)pyrrolo[2,1-f][1,2,4]triazin-4-yl)phenyl)hexahydropyrrolo[3,4-b]pyrrole-1(2H)-carboxylate (90c)

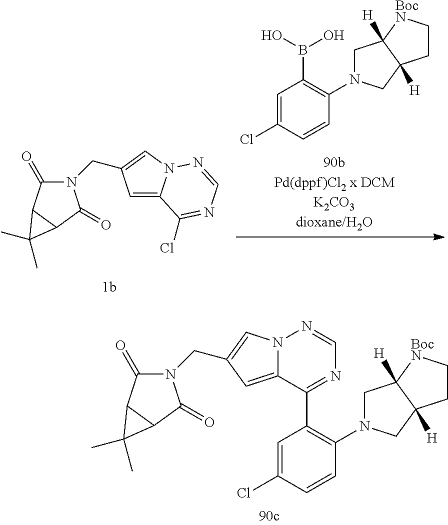 [SMILES: CC(C)(C)OC(=O)N1CC[C@@H]2CN(c3ccc(Cl)cc3B(O)O)C[C@@H]21 ; CC1(C)C2C(=O)N(Cc3cc4c(Cl)ncnn4c3)C(=O)C21 ; CC(C)(C)OC(=O)N1CC[C@@H]2CN(c3ccc(Cl)cc3-c3ncnn4cc(CN5C(=O)C6C(C5=O)C6(C)C)cc34)C[C@@H]21]

The title compound (90c) was obtained from 1b (75 mg; 0.245 mmol) and from 90b (the crude product) according to the General Procedure IV in 31% yield (per two steps)(36 mg; 0.061 mmol). The crude product was purified by flash column chromatography on silica (column 4 g, hexane/AcOEt, 100:0 to 0:100, v/v, 20 min, 10 mL/min).

ESI-MS m/z for C₃₁H₃₆ClN₆O₄ found 591.0/593.0 [M+H]⁺; Rₜ=1.76 min (Method A)

### Step 4.

### Synthesis of 3-((4-(5-chloro-2-((3aR,6aR)-hexahydropyrrolo[3,4-b]pyrrol-5(1H)-yl)phenyl)pyrrolo[2,1-f][1,2,4]triazin-6-yl)methyl)-6,6-dimethyl-3-azabicyclo[3.1.0]hexane-2,4-dione hydrochloride (90)

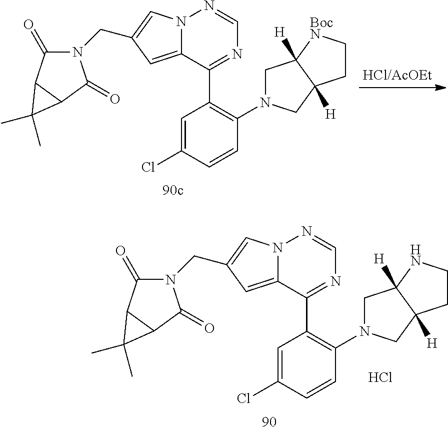 [SMILES: CC(C)(C)OC(=O)N1CC[C@@H]2CN(c3ccc(Cl)cc3-c3ncnn4cc(CN5C(=O)C6C(C5=O)C6(C)C)cc34)C[C@@H]21 ; CC1(C)C2C(=O)N(Cc3cc4c(-c5cc(Cl)ccc5N5C[C@H]6CCN[C@H]6C5)ncnn4c3)C(=O)C21 ; Cl]

The title compound (90) was obtained as a hydrochloride salt from 90c (32 mg; 0.055 mmol) according to the General Procedure IIIa in 80% yield (23 mg; 0.044 mmol). The crude product was purified by preparative reversed-phase column chromatography (C-18, water+0.3‰ TFA/MeCN, 98:2 to 30:70, 30 min, 20 mL/min, Rₜ=16.91 min).

ESI-MS m/z for C₂₆H₂₈ClN₆O₂ found 491.4/493.4 [M+H]⁺; Rₜ=1.12 min (Method A); ¹H NMR (700 MHz, D₂O) δ 8.54 (s, 1H), 8.13 (s, 1H), 7.59-7.54 (m, 1H), 7.39 (d, J=2.5 Hz, 1H), 7.17 (d, J=8.9 Hz, 1H), 6.86 (s, 1H), 4.72 (s, 2H), 4.31-4.25 (m, 1H), 3.40-3.35 (m, 1H), 3.30-3.24 (m, 1H), 3.23-3.17 (m, 1H), 3.01-2.92 (m, 2H), 2.92-2.86 (m, 1H), 2.75-2.70 (m, 1H), 2.63 (s, 2H), 2.13-2.05 (m, 1H), 1.46-1.38 (m, 1H), 1.24 (s, 3H), 1.00 (s, 3H).

### Example 91

### Synthesis of 3-((4-(5-chloro-2-(1,7-diazaspiro[4.4]nonan-7-yl)phenyl)pyrrolo[2,1-f][1,2,4]triazin-6-yl)methyl)-6,6-dimethyl-3-azabicyclo[3.1.0]hexane-2,4-dione hydrochloride (91)

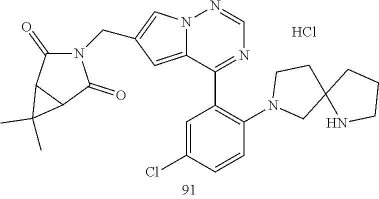 [SMILES: CC1(C)C2C(=O)N(Cc3cc4c(-c5cc(Cl)ccc5N5CCC6(CCCN6)C5)ncnn4c3)C(=O)C21 ; Cl]

The title compound (91) was obtained as a hydrochloride salt in 17% overall yield in a similar way to Example 90 with the exception that, in the first step of the synthesis, tert-butyl 1,7-diazaspiro[4.4]nonane-1-carboxylate was used instead of tert-butyl (3aR,6aR)-hexahydropyrrolo[3,4-b]pyrrole-1(2H)-carboxylate, and in the last step of the synthesis, the crude product was purified by preparative reversed-phase column chromatography (C-18, water+0.3‰ HCl (36%)/MeCN, 98:2 to 30:70, 30 min, 20 mL/min, Rₜ=17.42 min).

ESI-MS m/z for C₂₇H₃₀ClN₆O₂ found 505.4/507.4 [M+H]⁺; Rₜ=1.15 min (Method A); ¹H NMR (700 MHz, D₂O) δ 8.50 (s, 1H), 8.10 (s, 1H), 7.54-7.45 (m, 1H), 7.36 (d, J=2.6 Hz, 1H), 7.05 (d, J=9.1 Hz, 1H), 6.87 (s, 1H), 4.70 (s, 2H), 3.45-3.36 (m, 1H), 3.36-3.28 (m, 2H), 3.22-3.13 (m, 2H), 3.09-3.02 (m, 1H), 2.63 (s, 2H), 2.22-2.14 (m, 1H), 2.14-2.07 (m, 2H), 2.07-1.92 (m, 3H), 1.25 (s, 3H), 1.02 (s, 3H).

### Example 92

### Synthesis of 3-((4-(6-chloro-3-(pyrrolidin-3-yl)-1a,2,3,7b-tetrahydro-1H-cyclopropa[c]quinolin-4-yl)pyrrolo[2,1-f][1,2,4]triazin-6-yl)methyl)-6,6-dimethyl-3-azabicyclo[3.1.0]hexane-2,4-dione hydrochloride (92)

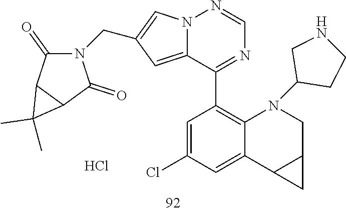 [SMILES: CC1(C)C2C(=O)N(Cc3cc4c(-c5cc(Cl)cc6c5N(C5CCNC5)CC5CC65)ncnn4c3)C(=O)C21 ; Cl]

The title compound (92) was obtained as a mixture of diastereoisomers as a hydrochloride salt in 5% overall yield in a similar way to Example 51 with the exception that, in the first step of the synthesis, 6-chloro-1a,2,3,7b-tetrahydro-1H-cyclopropa[c]quinoline (92d) (the synthesis of this compound was described below) was used instead of 6-chloro-1,2,3,4-tetrahydroquinoline, in the second step of the synthesis, acetic acid was added to the reaction mixture (14 equivalents) and in the last step of the synthesis, the General Procedure IIIa was used instead of the General Procedure IIIb and the crude product was purified by preparative reversed-phase column chromatography (C-18, water+0.23‰ HCl (36%)/MeCN, 98:2 to 5:95, 30 min, 20 mL/min, Rₜ=15.55 min).

ESI-MS m/z for C₂₈H₃₀ClN₆O₂ found 517.1/519.1 [M+H]⁺; Rₜ=1.19 min (Method C); ¹H NMR (400 MHz, Methanol-d₄) δ 8.66 (d, J=2.1 Hz, 1H), 8.34 (d, J=1.4 Hz, 1H), 7.57-7.50 (m, 1H), 7.30 (d, J=2.5 Hz, 1H), 7.04-6.92 (m, 1H), 4.76-4.63 (m, 2H), 3.92-3.78 (m, 1H), 3.40-3.33 (m, 1H), 3.29-3.24 (m, 1H), 3.19-3.05 (m, 1H), 2.95-2.85 (m, 1H), 2.54-2.43 (m, 2H), 2.21-1.99 (m, 2H), 1.99-1.61 (m, 2H), 1.45-1.28 (m, 1H), 1.27-1.21 (m, 3H), 1.22-1.15 (m, 1H), 1.15-1.09 (m, 3H), 0.96-0.59 (m, 2H).

### Synthesis of 6-chloro-1a,2,3,7b-tetrahydro-1H-cyclopropa[c]quinoline (92d)

### Step 1.

### Synthesis of 6-chloro-1-(4-methoxybenzyl)quinolin-2(1H)-one (92a)

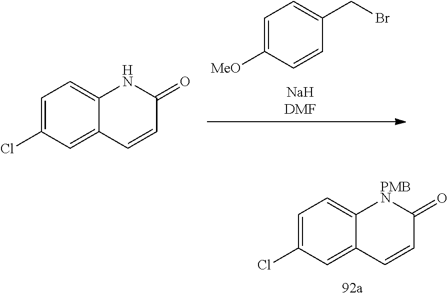

To the cooled to 0° C. solution of 6-chloroquinolin-2(1H)-one (772 mg; 4.21 mmol) in dry DMF (5 mL) NaH (60% in mineral oil; 202 mg; 5.05 mmol) was added under an Argon atmosphere and a whole was then stirred for 30 minutes at room temperature. Then to this mixture a solution of 1-(bromomethyl)-4-methoxybenzene (847 mg; 4.21 mmol) in DMF (3 mL) was added dropwise and the reaction mixture was stirred at room temperature for 2 days. The reaction progress was monitored by LC-MS. When analysis indicated completion of the reaction, DMF was evaporated in vacuo and to the residue water wad added and then was extracted with AcOEt. The organic layer was washed with brine, then dried over anhydrous Na₂SO₄, filtered and concentrated in vacuo. The crude product was then purified by flash column chromatography on silica (hexane/AcOEt, 100:0 to 20:80, v/v, 27 min, 40 mL/min). Compound 92a was obtained in 76% yield (954 mg; 3.19 mmol).

ESI-MS m/z for C₁₇H₁₅ClNO₂ found 299.9/302.0 [M+H]⁺; Rₜ=1.46 min (Method A)

### Step 2.

### Synthesis of 6-chloro-3-(4-methoxybenzyl)-1,1a,3,7b-tetrahydro-2H-cyclopropa[c]quinolin-2-one (92b)

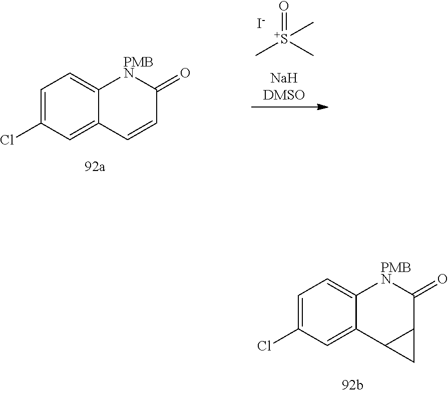

To the solution of NaH (60% in mineral oil; 40 mg; 1.00 mmol) in DMSO (1 mL) the compound 92a (200 mg; 0.67 mmol) was added at room temperature, followed by trimethylsulfoxonium iodide (220 mg; 1.00 mmol) and a whole was then stirred overnight at 45° C. The reaction progress was monitored by LC-MS. When analysis indicated completion of the reaction, the reaction mixture was diluted with AcOEt and quenched with water. The phases were separated and an aqueous one (pH=8/9) was extracted with AcOEt (5×). The combined organic phases were dried over anhydrous Na₂SO₄, filtered and concentrated in vacuo. The crude product was then purified by flash column chromatography on silica (hexane/AcOEt, 100:0 to 40:60, v/v, 30 min, 20 mL/min). Compound 92b was obtained in 94% yield (196 mg; 0.63 mmol).

ESI-MS m/z for C₁₈H₁₇ClNO₂ found 314.0/316.0 [M+H]⁺; Rₜ=1.53 min (Method C); ¹H NMR (400 MHz, CDCl₃) δ 7.34-7.30 (m, 1H), 7.10-7.05 (m, 2H), 7.04-6.99 (m, 1H), 6.86-6.80 (m, 2H), 6.78-6.74 (m, 1H), 5.27-4.93 (m, 2H), 3.76 (s, 3H), 2.55-2.38 (m, 2H), 1.72-1.63 (m, 1H), 0.79-0.63 (m, 1H).

### Step 3.

### Synthesis of 6-chloro-1,1a,3,7b-tetrahydro-2H-cyclopropa[c]quinolin-2-one (92c)

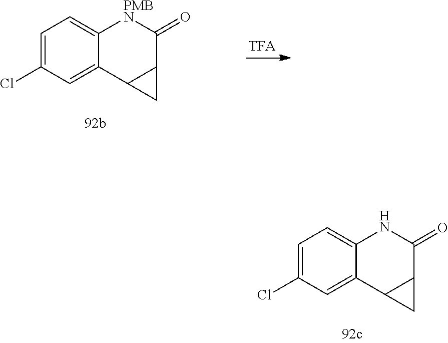

The solution of the compound 92b (956 mg; 3.05 mmol) and TFA (8 mL; 104.00 mmol) was stirred overnight at 65° C. The reaction progress was monitored by LC-MS. When analysis indicated completion of the reaction, the reaction mixture was diluted with DCM and quenched with an aqueous solution of NaHCO₃. The phases were separated and an aqueous one was extracted with DCM (3×). The combined organic phases were dried over anhydrous Na₂SO₄, filtered and concentrated in vacuo. The crude product was used to the next step without any additional purification.

ESI-MS m/z for C₁₀H₉ClNO found 194.0/196.0 [M+H]⁺; Rₜ=1.01 min (Method C)

### Step 4.

### Synthesis of 6-chloro-1a,2,3,7b-tetrahydro-1H-cyclopropa[c]quinoline (92d)

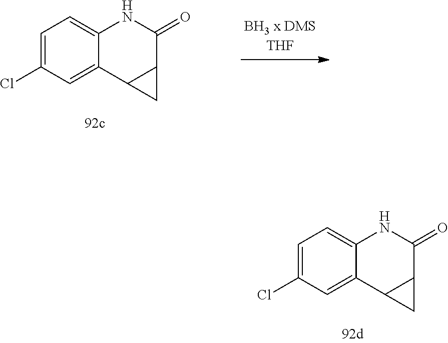 [SMILES: O=C1Nc2ccc(Cl)cc2C2CC12 ; O=C1Nc2ccc(Cl)cc2C2CC12]

To the cooled to 0° C. solution of 92c (the crude product) in dry THF (10.2 mL) BH₃×DMS (0.58 mL; 6.10 mmol) was slowly added and this mixture was then stirred at 80° C. for 2 hours. The reaction progress was monitored by LC-MS. When analysis indicated completion of the reaction, the reaction mixture was cooled to 0° C. and to this mixture MeOH was added and a whole was stirred at room temperature for 20 minutes. Then the solvents were removed in vacuo and the residue was purified by flash column chromatography on silica (hexane/AcOEt, 100:0 to 70:30, v/v, 30 min, 26 mL/min). Compound 92d was obtained in 73% yield (per two steps)(400 mg; 2.23 mmol).

ESI-MS m/z for C₁₀H₁₁ClN found 180.0/182.0 [M+H]⁺; Rₜ=1.33 min (Method C)

### Example 93

### Synthesis of 3-((4-((2R)-7-chloro-2-methyl-4-(pyrrolidin-3-yl)-3,4-dihydro-2H-benzo[b][1,4]oxazin-5-yl)pyrrolo[2,1-f][1,2,4]triazin-6-yl)methyl)-6,6-dimethyl-3-azabicyclo[3.1.0]hexane-2,4-dione hydrochloride (93)

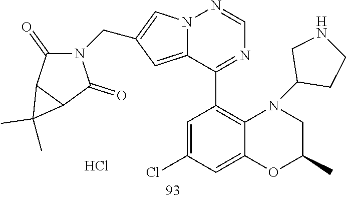 [SMILES: C[C@@H]1CN(C2CCNC2)c2c(cc(Cl)cc2-c2ncnn3cc(CN4C(=O)C5C(C4=O)C5(C)C)cc23)O1 ; Cl]

The title compound (93) was obtained as a mixture of diastereoisomers as a hydrochloride salt in 13% overall yield in a similar way to Example 51 with the exception that, in the first step of the synthesis, (R)-7-chloro-2-methyl-3,4-dihydro-2H-benzo[b][1,4]oxazine (93c) (the synthesis of this compound was described below) was used instead of 6-chloro-1,2,3,4-tetrahydroquinoline, and in the last step of the synthesis, the General Procedure IIIa was used instead of the General Procedure IIIb and the crude product was purified by preparative reversed-phase column chromatography (C-18, water+1‰ HCl (36%)/MeCN, 98:2 to 5:95, 30 min, 20 mL/min, Rₜ=15.32 min).

ESI-MS m/z for C₂₇H₃₀ClN₆O₃ found 521.1/523.1 [M+H]⁺; Rₜ=1.19 min (Method C); ¹H NMR (400 MHz, Methanol-d₄) δ 8.59-8.54 (m, 1H), 8.07-8.04 (m, 1H), 7.10-7.05 (m, 2H), 6.78-6.71 (m, 1H), 4.70-4.60 (m, 2H), 4.10-3.97 (m, 1H), 3.78-3.65 (m, 1H), 3.48-3.37 (m, 1H), 3.22-2.55 (m, 4H), 2.51-2.44 (m, 2H), 1.94-1.56 (m, 2H), 1.47-1.40 (m, 3H), 1.36-1.27 (m, 1H), 1.27-1.20 (m, 3H), 1.12-1.05 (m, 3H), 0.97-0.85 (m, 1H).

### Synthesis of (R)-7-chloro-2-methyl-3,4-dihydro-2H-benzo[b][1,4]oxazine (93c)

### Step 1.

### Synthesis of (S)—N-(4-chloro-2-hydroxyphenyl)-2-hydroxypropanamide (93a)

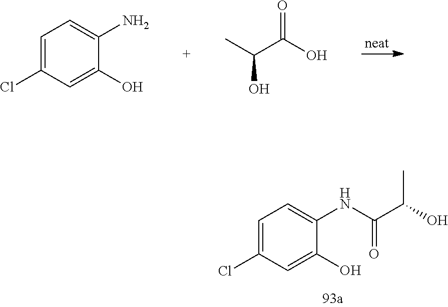

The mixture of 2-amino-5-chlorophenol (1.00 g; 6.756 mmol) and (S)-2-hydroxypropanoic acid (0.637 mL; 8.110 mmol) was stirred at 80° C. for 2 days. The reaction progress was monitored by LC-MS. When analysis indicated completion of the reaction, the reaction mixture was diluted with AcOEt and water (pH=2). An aqueous layer was neutralized with an aqueous solution of NaHCO₃ and then extracted with AcOEt (3×). The combined organic phases were dried over anhydrous Na₂SO₄, filtered and concentrated in vacuo. The crude product was purified by flash column chromatography on silica (hexane/AcOEt, 100:0 to 40:60, v/v, 30 min, 26 mL/min). Compound 93a was obtained in 57% yield (829 mg; 3.855 mmol).

ESI-MS m/z for C₉H₁₁ClNO₃ found 216.0/218.0 [M+H]⁺; Rₜ=0.85 min (Method C)

### Step 2.

### Synthesis of (R)-7-chloro-2-methyl-2H-benzo[b][1,4]oxazin-3(4H)-one (93b)

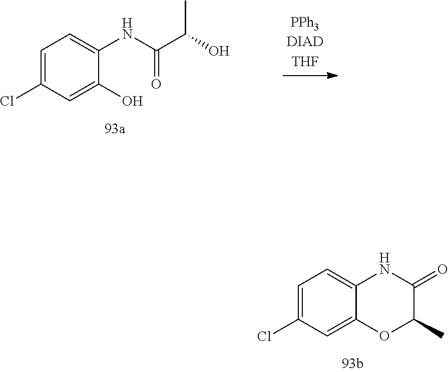

To a cooled to −10° C. solution of 93a (829 mg; 3.855 mmol) in THF (13 mL) PPh₃ (1.274 g; 4.61 mmol) was added followed by DIAD (0.98 mL; 5.000 mmol) and the resulting mixture was stirred at room temperature for 1.15 hour. The reaction progress was monitored by LC-MS. When analysis indicated completion of the reaction, the reaction mixture was diluted with AcOEt and quenched with water (pH=7). An aqueous layer was extracted with AcOEt (3×). The combined organic phases were dried over anhydrous Na₂SO₄, filtered and concentrated in vacuo. The crude product was purified by flash column chromatography on silica (hexane/AcOEt, 100:0 to 40:60, v/v, 30 min, 34 mL/min). Compound 93b was obtained in 99% yield (752 mg; 3.816 mmol).

ESI-MS m/z for C₉H₉ClNO₂ found 197.9/200.0 [M+H]⁺; Rₜ=1.37 min (Method C)

### Step 3.

### Synthesis of (R)-7-chloro-2-methyl-3,4-dihydro-2H-benzo[b][1,4]oxazine (93c)

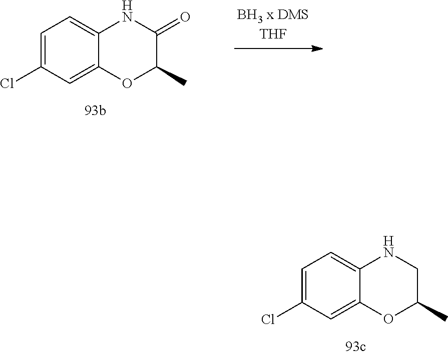

To the cooled to 0° C. solution of 93b (1.290 g; 6.528 mmol) in dry THE (22.6 mL) BH₃×DMS (1.24 mL; 13.100 mmol) was slowly added and this mixture was then warmed to room temperature and stirred for 1 hour and then stirred at 80° C. for 1 hour. The reaction progress was monitored by LC-MS. When analysis indicated completion of the reaction, the reaction mixture was cooled to 0° C. and to this mixture MeOH was added and a whole was stirred at this temperature for 20 minutes. Then the solvents were removed in vacuo and the residue was redissolved in AcOEt and to this solution water was added. An aqueous layer was extracted with AcOEt (3×). The combined organic phases were dried over anhydrous Na₂SO₄, filtered and concentrated in vacuo. The crude product was purified by flash column chromatography on silica (hexane/AcOEt, 100:0 to 0:100, v/v, 30 min, 26 mL/min). Compound 93c was obtained in 36% yield (432 mg; 2.360 mmol).

ESI-MS m/z for C₉H₁₁ClNO found 184.0/186.0 [M+H]⁺; Rₜ=1.28 min (Method C)

### Example 94

### Synthesis of 3-((4-((2S)-7-chloro-2-methyl-4-(pyrrolidin-3-yl)-3,4-dihydro-2H-benzo[b][1,4]oxazin-5-yl)pyrrolo[2,1-f][1,2,4]triazin-6-yl)methyl)-6,6-dimethyl-3-azabicyclo[3.1.0]hexane-2,4-dione hydrochloride (94)

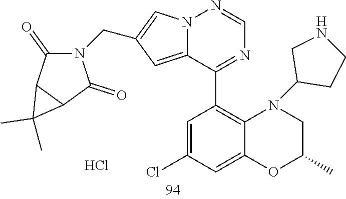 [SMILES: C[C@H]1CN(C2CCNC2)c2c(cc(Cl)cc2-c2ncnn3cc(CN4C(=O)C5C(C4=O)C5(C)C)cc23)O1 ; Cl]

The title compound (94) was obtained as a mixture of diastereoisomers as a hydrochloride salt in 7% overall yield in a similar way to Example 51 with the exception that, in the first step of the synthesis, (S)-7-chloro-2-methyl-3,4-dihydro-2H-benzo[b][1,4]oxazine (the synthesis of this compound was the same as for 93c with the exception, that in the first step of this synthesis, (R)-2-hydroxypropanoic acid was used instead of (S)-2-hydroxypropanoic acid) was used instead of 6-chloro-1,2,3,4-tetrahydroquinoline, and in the last step of the synthesis, the General Procedure IIIa was used instead of the General Procedure IIIb and the crude product was purified by preparative reversed-phase column chromatography (C-18, water+1‰ HCl (36%)/MeCN, 95:5 to 5:95, 30 min, 20 mL/min, Rₜ=15.50 min).

ESI-MS m/z for C₂₇H₃₀ClN₆O₃ found 521.1/523.1 [M+H]⁺; Rₜ=1.18 min (Method C); ¹H NMR (400 MHz, Methanol-d₄) δ 8.64-8.60 (m, 1H), 8.25-8.21 (m, 1H), 7.18-7.09 (m, 2H), 7.04-6.95 (m, 1H), 4.78-4.65 (m, 2H), 4.18-4.02 (m, 1H), 3.85-3.67 (m, 1H), 3.54-3.45 (m, 1H), 3.24-2.62 (m, 4H), 2.54-2.47 (m, 2H), 2.07-1.60 (m, 2H), 1.52-1.42 (m, 3H), 1.34-1.24 (m, 4H), 1.16-1.10 (m, 3H), 1.06-0.91 (m, 1H).

### Example 95

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1-(2,2-difluoroethyl)pyrimidine-2,4(1H,3H)-dione hydrochloride (95)

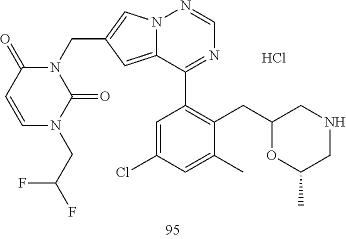 [SMILES: Cc1cc(Cl)cc(-c2ncnn3cc(Cn4c(=O)ccn(CC(F)F)c4=O)cc23)c1CC1CNC[C@H](C)O1 ; Cl]

The title compound (95) was obtained as a hydrochloride salt in 10% overall yield in a similar way to Example 59 with the exception that, in the second step of the synthesis, 1-(2,2-difluoroethyl)pyrimidine-2,4(1H,3H)-dione (the synthesis of this compound was described below) was used instead of 1-methylpyrimidine-2,4(1H,3H)-dione and in the last step of the synthesis, the crude product was purified by preparative reversed-phase column chromatography (column: Cosmosil Cholester 20×250 mm, water+0.3‰ HCl (36%)/MeCN, 99:1 to 20:80, 30 min, 20 mL/min, Rₜ=16.00 min).

ESI-MS m/z for C₂₆H₂₈ClF₂N₆O₃ found 545.2/547.2 [M+H]⁺; Rₜ=0.97 min (Method A); ¹H NMR (700 MHz, D₂O) δ 8.38 (s, 1H), 8.00 (s, 1H), 7.53 (d, J=7.9 Hz, 1H), 7.42 (d, J=1.9 Hz, 1H), 7.28 (d, J=1.9 Hz, 1H), 6.65 (s, 1H), 6.18-6.00 (m, 1H), 5.81 (d, J=7.9 Hz, 1H), 5.12-5.07 (m, 2H), 4.21-4.15 (m, 2H), 3.63-3.60 (m, 1H), 3.34-3.31 (m, 1H), 3.15-3.09 (m, 2H), 2.97 (d, J=12.4 Hz, 1H), 2.92-2.87 (m, 1H), 2.53-2.49 (m, 1H), 2.35-2.30 (m, 4H), 0.40 (s, 3H).

### Synthesis of 1-(2,2-difluoroethyl)pyrimidine-2,4(1H,3H)-dione

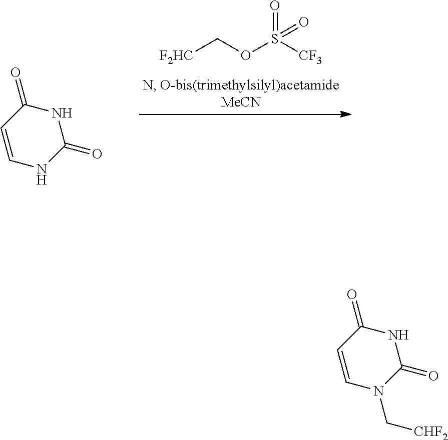 [SMILES: O=S(=O)(OCC(F)F)C(F)(F)F ; O=c1cc[nH]c(=O)[nH]1 ; O=c1ccn(CC(F)F)c(=O)[nH]1]

To the solution of pyrimidine-2,4(1H,3H)-dione (1.00 g; 8.65 mmol) in dry MeCN (81 mL) N,O-bis(trimethylsilyl)acetamide (5.44 g; 26.00 mmol) was added and this mixture was then refluxed for 2 hours. Then the reaction mixture was cooled to room temperature and to this mixture 2,2-difluoroethyl trifluoromethanesulfonate (4.00 g; 18.70 mmol) was added and then stirred at 60° C. for 2 days. The reaction progress was monitored by TLC. When analysis indicated completion of the reaction, the reaction mixture was cooled to room temperature and the solvent was removed in vacuo and the residue was purified by flash column chromatography on silica (DCM/MeOH, 100:0 to 50:50, v/v, 30 min, 30 mL/min). 1-(2,2-difluoroethyl)pyrimidine-2,4(1H,3H)-dione was obtained in 44% yield (670 mg; 3.81 mmol).

### Example 96

### Synthesis of 3-((4-(5-chloro-2-((3aS,6aS)-hexahydropyrrolo[3,4-b]pyrrol-5(1H)-yl)phenyl)pyrrolo[2,1-f][1,2,4]triazin-6-yl)methyl)-6,6-dimethyl-3-azabicyclo[3.1.0]hexane-2,4-dione hydrochloride (96)

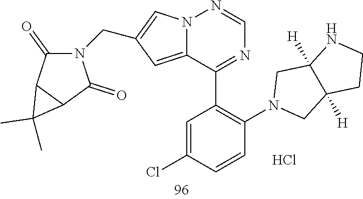 [SMILES: CC1(C)C2C(=O)N(Cc3cc4c(-c5cc(Cl)ccc5N5C[C@@H]6CCN[C@@H]6C5)ncnn4c3)C(=O)C21 ; Cl]

The title compound (96) was obtained as a hydrochloride salt in 4% overall yield in a similar way to Example 90 with the exception that, in the first step of the synthesis, tert-butyl (3aS,6aS)-hexahydropyrrolo[3,4-b]pyrrole-1(2H)-carboxylate was used instead of tert-butyl (3aR,6aR)-hexahydropyrrolo[3,4-b]pyrrole-1(2H)-carboxylate and in the last step of the synthesis, the crude product was purified by preparative reversed-phase column chromatography (C-18, water+0.3‰ HCl (36%)/MeCN, 98:2 to 30:70, 30 min, 20 mL/min, Rₜ=17.63 min).

ESI-MS m/z for C₂₆H₂₈ClN₆O₂ found 491.4/493.4 [M+H]⁺; Rₜ=1.07 min (Method A); ¹H NMR (700 MHz, D₂O) δ 8.53 (s, 1H), 8.11 (s, 1H), 7.57-7.52 (m, 1H), 7.36 (d, J=2.4 Hz, 1H), 7.16 (d, J=8.9 Hz, 1H), 6.83 (s, 1H), 4.71 (s, 2H), 4.30-4.27 (m, 1H), 3.39-3.35 (m, 1H), 3.29-3.25 (m, 1H), 3.23-3.18 (m, 1H), 2.99-2.93 (m, 2H), 2.91-2.86 (m, 1H), 2.74-2.71 (m, 1H), 2.64-2.61 (m, 2H), 2.13-2.06 (m, 1H), 1.45-1.40 (m, 1H), 1.25 (s, 3H), 1.01 (s, 3H).

### Example 97

### Synthesis of 3-((4-(5-chloro-2-(hexahydropyrrolo[3,4-c]pyrrol-2(1H)-yl)phenyl)pyrrolo[2,1-f][1,2,4]triazin-6-yl)methyl)-6,6-dimethyl-3-azabicyclo[3.1.0]hexane-2,4-dione hydrochloride (97)

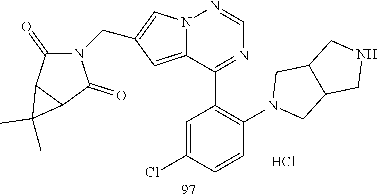

The title compound (97) was obtained as a hydrochloride salt in 21% overall yield in a similar way to Example 90 with the exception that, in the first step of the synthesis, tert-butyl hexahydropyrrolo[3,4-c]pyrrole-2(1H)-carboxylate was used instead of tert-butyl (3aR,6aR)-hexahydropyrrolo[3,4-b]pyrrole-1(2H)-carboxylate, and in the last step of the synthesis, the crude product was purified by preparative reversed-phase column chromatography (C-18, water+0.3‰ HCl (36%)/MeCN, 98:2 to 30:70, 30 min, 20 mL/min, Rₜ=17.33 min).

ESI-MS m/z for C₂₆H₂₈ClN₆O₂ found 491.4/493.4 [M+H]⁺; Rₜ=1.13 min (Method A); ¹H NMR (700 MHz, D₂O) δ 8.53 (s, 1H), 8.09 (s, 1H), 7.56-7.53 (m, 1H), 7.37 (d, J=2.5 Hz, 1H), 7.18 (d, J=8.9 Hz, 1H), 6.82 (s, 1H), 4.72 (s, 2H), 3.52-3.47 (m, 2H), 3.02-2.94 (m, 4H), 2.93-2.89 (m, 2H), 2.63 (s, 2H), 2.61-2.56 (m, 2H), 1.25 (s, 3H), 1.01 (s, 3H).

### Example 98

### Synthesis of (R)-1-((4-(5-chloro-3-methyl-2-(morpholin-2-ylmethyl)phenyl)pyrrolo[2,1-f][1,2,4]triazin-6-yl)methyl)-5-fluoro-3-methylpyrimidine-2,4(1H,3H)-dione hydrochloride (98)

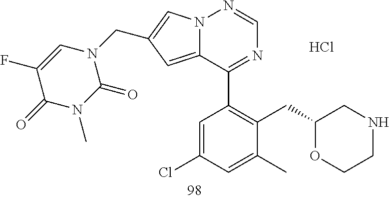 [SMILES: Cc1cc(Cl)cc(-c2ncnn3cc(Cn4cc(F)c(=O)n(C)c4=O)cc23)c1C[C@@H]1CNCCO1 ; Cl]

### Step 1.

### Synthesis of 1-((4-chloropyrrolo[2,1-f][1,2,4]triazin-6-yl)methyl)-5-fluoropyrimidine-2,4(1H,3H)-dione (98a)

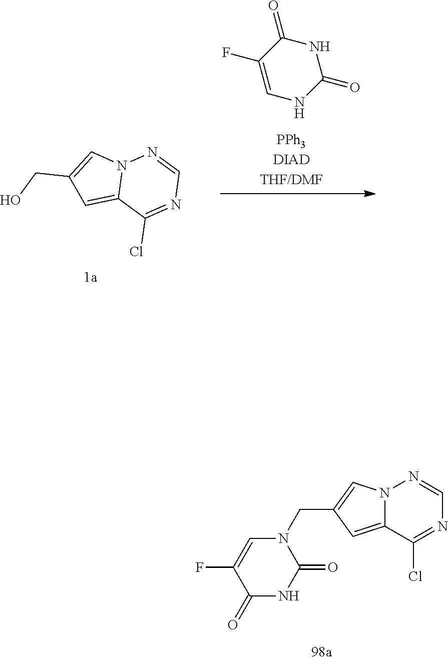 [SMILES: O=c1[nH]cc(F)c(=O)[nH]1 ; OCc1cc2c(Cl)ncnn2c1 ; O=c1[nH]c(=O)n(Cc2cc3c(Cl)ncnn3c2)cc1F]

To a cooled to −10° C. solution of 1a (200 mg; 1.09 mmol), 5-fluoropyrimidine-2,4(1H,3H)-dione (132 mg; 0.98 mmol) and PPh₃ (343 mg; 1.31 mmol) in THF/DMF (2 mL/2 mL) DIAD (0.28 mL; 1.42 mmol) was added dropwise. The resulting mixture was stirred at room temperature for 2 hours. Then the reaction mixture was concentrated in vacuo and the residue was filtered and the precipitate was washed with Et₂O (3×10 mL). The combined filtrate was concentrated in vacuo and then purified by flash column chromatography on silica (DCM, 100%, 5 min, then DCM/MeOH, 100:0 to 80:20, v/v, 15 min, 13 mL/min). Then the crude product was washed with AcOEt (2×1 mL) and Et₂O (2×1 mL) and the compound 98a was obtained as a white solid in 25% yield (75 mg; 0.25 mmol).

ESI-MS m/z for C₁₁H₈ClFN₅O₂ found 295.9/297.6 [M+H]⁺; Rₜ=0.74 min (Method A); ¹H NMR (700 MHz, DMSO-d₆) δ 8.42 (s, 1H), 8.25 (d, J=1.6 Hz, 1H), 8.20 (d, J=6.7 Hz, 1H), 7.08 (d, J=1.6 Hz, 1H), 4.93 (s, 2H).

### Step 2.

### Synthesis of tert-butyl (R)-2-(4-chloro-2-(6-((5-fluoro-2,4-dioxo-3,4-dihydropyrimidin-1(2H)-yl)methyl)pyrrolo[2,1-f][1,2,4]triazin-4-yl)-6-methylbenzyl)morpholine-4-carboxylate (98b)

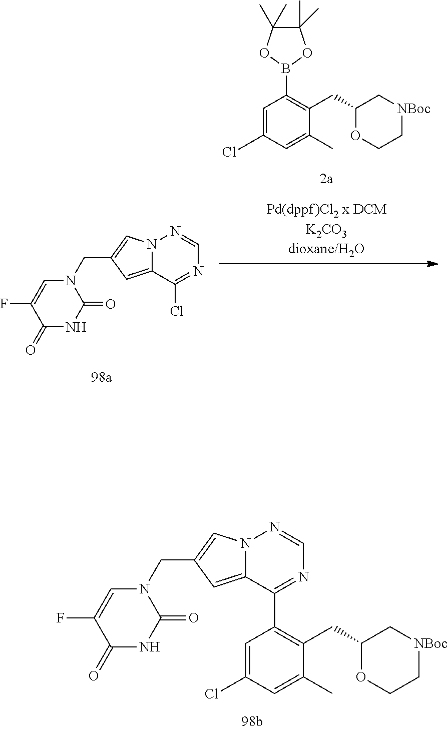 [SMILES: Cc1cc(Cl)cc(B2OC(C)(C)C(C)(C)O2)c1C[C@@H]1CN(C(=O)OC(C)(C)C)CCO1 ; O=c1[nH]c(=O)n(Cc2cc3c(Cl)ncnn3c2)cc1F ; Cc1cc(Cl)cc(-c2ncnn3cc(Cn4cc(F)c(=O)[nH]c4=O)cc23)c1C[C@@H]1CN(C(=O)OC(C)(C)C)CCO1]

The title compound (98b) was obtained from 98a (33 mg; 0.111 mmol) and from 2a (obtained according to the procedure described in the example 82)(50 mg; 0.111 mmol) according to the General Procedure IV in 52% yield (34 mg; 0.058 mmol). The crude product was purified by flash column chromatography on silica (hexane, 100%, 2 min, then hexane/AcOEt, 100:0 to 0:100, v/v, 8 min, then AcOEt, 100%, 10 min, 15 mL/min).

ESI-MS m/z for C₂₈H₃₁ClFN₆O₅ found 585.1/587.1 [M+H]⁺; Rₜ=1.53 min (Method A)

### Step 3.

### Synthesis of tert-butyl (R)-2-(4-chloro-2-(6-((5-fluoro-3-methyl-2,4-dioxo-3,4-dihydropyrimidin-1(2H)-yl)methyl)pyrrolo[2,1-f][1,2,4]triazin-4-yl)-6-methylbenzyl)morpholine-4-carboxylate (98c)

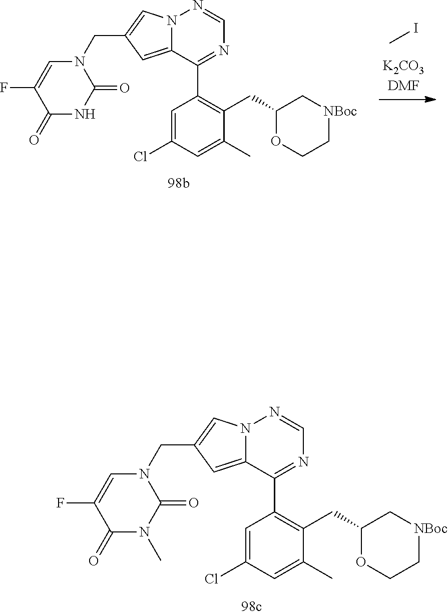

To the solution of 98b (30 mg; 0.051 mmol) in dry DMF (1 mL) K₂CO₃ (14.5 mg; 0.103 mmol) and methyl iodide (3.2 μL; 0.051 mmol) were added. The resulting mixture was stirred at room temperature for 5 hours. The reaction progress was monitored by LC-MS. When analysis indicated completion of the reaction, the reaction mixture was diluted with AcOEt (15 mL) and washed with water (10 mL). The organic layer was then dried over anhydrous Na₂SO₄, filtered and concentrated in vacuo and the residue was purified by flash column chromatography on silica (hexane, 100%, 1 min, then hexane/AcOEt, 100:0 to 0:100, v/v, 8 min, then AcOEt, 100%, 7 min, 15 mL/min). Compound 98c was obtained in 74% yield (23 mg; 0.038 mmol).

ESI-MS m/z for C₂₉H₃₃ClFN₆O₅ found 599.1/601.0 [M+H]⁺; Rₜ=1.68 min (Method A)

### Step 4.

### Synthesis of (R)-1-((4-(5-chloro-3-methyl-2-(morpholin-2-ylmethyl)phenyl)pyrrolo[2,1-f][1,2,4]triazin-6-yl)methyl)-5-fluoro-3-methylpyrimidine-2,4(1H,3H)-dione hydrochloride (98)

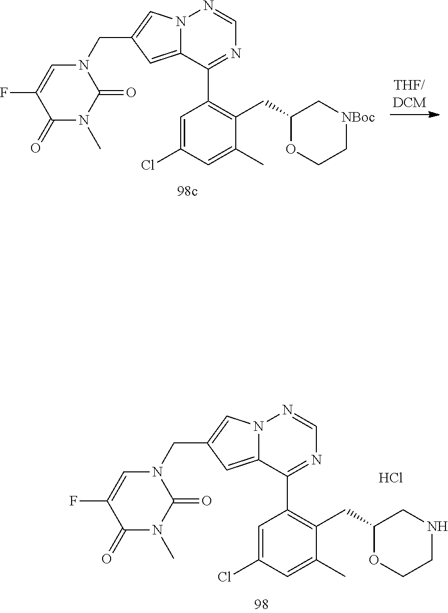 [SMILES: Cc1cc(Cl)cc(-c2ncnn3cc(Cn4cc(F)c(=O)n(C)c4=O)cc23)c1C[C@@H]1CN(C(=O)OC(C)(C)C)CCO1 ; Cc1cc(Cl)cc(-c2ncnn3cc(Cn4cc(F)c(=O)n(C)c4=O)cc23)c1C[C@@H]1CNCCO1 ; Cl]

The title compound (98) was obtained as a single enantiomer (99% ee) as a hydrochloride salt from 98c (22 mg; 0.037 mmol) according to the General Procedure IIIb in 38% yield (7.6 mg; 0.014 mmol). The crude product was purified by preparative reversed-phase column chromatography (C-18, water+0.3‰ HCl (36%)/MeCN, 98:2 to 30:70, 30 min, 15 mL/min, Rₜ=17.00 min).

ESI-MS m/z for C₂₄H₂₅ClFN₆O₃ found 499.4/501.4 [M+H]⁺; Rₜ=1.00 min (Method A); ¹H NMR (700 MHz, Methanol-d₄) δ 8.61 (s, 1H), 8.34 (d, J=1.5 Hz, 1H), 8.01 (d, J=5.8 Hz, 1H), 7.55-7.47 (m, 1H), 7.45-7.40 (m, 1H), 6.97 (d, J=1.5 Hz, 1H), 5.09 (s, 2H), 3.80-3.70 (m, 2H), 3.54-3.47 (m, 1H), 3.32-3.29 (m, 3H), 3.24-3.17 (m, 1H), 3.12-3.06 (m, 1H), 2.99-2.87 (m, 3H), 2.81-2.73 (m, 1H), 2.49 (s, 3H).

### Example 99

### Synthesis of (R)-3-((4-(5-chloro-3-methyl-2-(morpholin-2-ylmethyl)phenyl)pyrrolo[2,1-f][1,2,4]triazin-6-yl)methyl)-1-methylimidazolidine-2,4-dione hydrochloride (99)

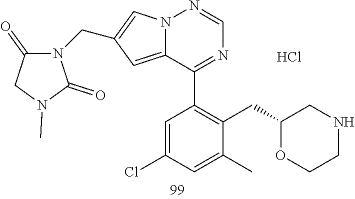 [SMILES: Cc1cc(Cl)cc(-c2ncnn3cc(CN4C(=O)CN(C)C4=O)cc23)c1C[C@@H]1CNCCO1 ; Cl]

The title compound (99) was obtained as a single enantiomer (99% ee) as a hydrochloride salt in 18% overall yield in a similar way to Example 36 with the exception that, in the first step of the synthesis, 1-methylimidazolidine-2,4-dione was used instead of 5,5-dimethylimidazolidine-2,4-dione. In the second step of the synthesis, the compound 2a, obtained according to the procedure described in the example 82, was used. In the last step of synthesis, the crude product was purified by preparative reversed-phase column chromatography (C-18, water+0.3‰ HCl (36%)/MeCN, 98:2 to 25:75, 30 min, 15 mL/min, Rₜ=15.4 min).

ESI-MS m/z for C₂₃H₂₆ClN₆O₃ found 469.5/471.5 [M+H]⁺; Rₜ=0.97 min (Method A); ¹H NMR (700 MHz, Methanol-d₄) δ 8.79 (s, 1H), 8.68 (d, J=1.4 Hz, 1H), 7.69-7.62 (m, 1H), 7.55-7.49 (m, 1H), 7.39-7.33 (m, 1H), 4.87-4.84 (m, 2H), 4.01 (s, 2H), 3.79-3.70 (m, 2H), 3.57-3.52 (m, 1H), 3.33-3.31 (m, 1H), 3.13-3.09 (m, 1H), 3.05-3.01 (m, 1H), 2.96 (s, 3H), 2.94-2.88 (m, 1H), 2.82-2.74 (m, 2H), 2.52 (s, 3H).

### Example 100

### Synthesis of (S)-3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione hydrochloride (100)

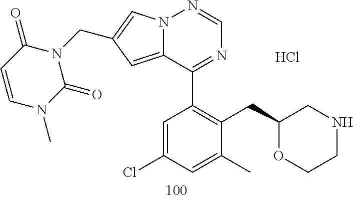 [SMILES: Cc1cc(Cl)cc(-c2ncnn3cc(Cn4c(=O)ccn(C)c4=O)cc23)c1C[C@H]1CNCCO1 ; Cl]

The title compound (100) was obtained as a single enantiomer (99% ee) as a hydrochloride salt in 1% overall yield in a similar way to Example 82 with the exception that, in the second step of the synthesis, the compound 82a′ was used instead of the compound 82a. In the last step of synthesis, the crude product was purified by preparative reversed-phase column chromatography (C-18, water+0.3‰ HCl (36%)/MeCN, 99:1 to 40:60, 30 min, 15 mL/min, Rₜ=17.6 min).

ESI-MS m/z for C₂₄H₂₆ClN₆O₃ found 481.4/483.4 [M+H]⁺; Rₜ=1.00 min (Method A); ¹H NMR (700 MHz, Methanol-d₄) δ 8.49 (s, 1H), 8.09 (d, J=1.5 Hz, 1H), 7.55 (d, J=7.8 Hz, 1H), 7.50-7.45 (m, 1H), 7.39-7.33 (m, 1H), 6.70 (d, J=1.5 Hz, 1H), 5.73 (d, J=7.8 Hz, 1H), 5.20 (s, 2H), 3.77-3.70 (m, 2H), 3.50-3.43 (m, 1H), 3.37 (s, 3H), 3.15-3.06 (m, 2H), 3.02-2.87 (m, 3H), 2.77-2.71 (m, 1H), 2.48 (s, 3H).

### Example 101

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1-(2-fluoroethyl)pyrimidine-2,4(1H,3H)-dione hydrochloride (101)

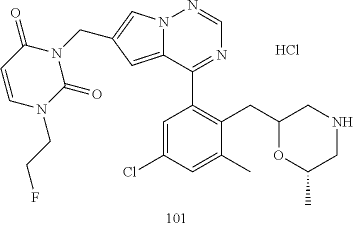 [SMILES: Cc1cc(Cl)cc(-c2ncnn3cc(Cn4c(=O)ccn(CCF)c4=O)cc23)c1CC1CNC[C@H](C)O1 ; Cl]

The title compound (95) was obtained as a hydrochloride salt in 8.5% overall yield in a similar way to Example 59 with the exception that, in the second step of the synthesis, 1-(2-fluoroethyl)pyrimidine-2,4(1H,3H)-dione (the synthesis of this compound was described below) was used instead of 1-methylpyrimidine-2,4(1H,3H)-dione and in the last step of the synthesis, the crude product was purified by preparative reversed-phase column chromatography (C-18, water+0.3‰ HCl (36%)/MeCN, 99:1 to 25:75, 30 min, 15 mL/min, Rₜ=17.20 min).

ESI-MS m/z for C₂₆H₂₉ClFN₆O₃ found 527.4/529.4 [M+H]⁺; Rₜ=1.07 min (Method A); ¹H NMR (700 MHz, Methanol-d₄) δ 8.55 (s, 1H), 8.22 (s, 1H), 7.56 (d, J=7.9 Hz, 1H), 7.52 (s, 1H), 7.45 (d, J=2.1 Hz, 1H), 6.89 (s, 1H), 5.75 (d, J=7.9 Hz, 1H), 5.26-5.18 (m, 2H), 4.69-4.64 (m, 1H), 4.61-4.57 (m, 1H), 4.14-4.10 (m, 1H), 4.10-4.06 (m, 1H), 3.79-3.75 (m, 1H), 3.51-3.46 (m, 1H), 3.20-3.17 (m, 1H), 3.15-3.11 (m, 1H), 3.07-3.04 (m, 1H), 2.97-2.93 (m, 1H), 2.68-2.63 (m, 1H), 2.55-2.49 (m, 1H), 2.48 (s, 3H), 0.72 (d, J=6.2 Hz, 3H).

### Synthesis of 1-(2-fluoroethyl)pyrimidine-2,4(1H,3H)-dione

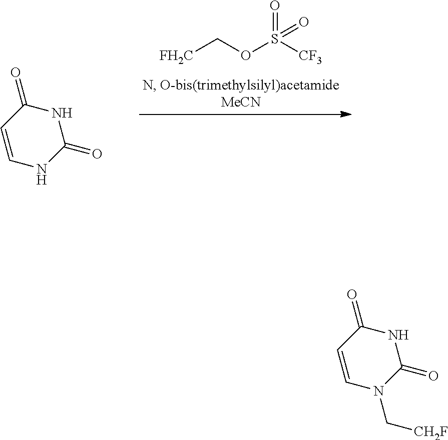

To the solution of pyrimidine-2,4(1H,3H)-dione (0.28 g; 2.42 mmol) in dry MeCN (23 mL) N,O-bis(trimethylsilyl)acetamide (1.52 g; 7.26 mmol) was added and this mixture was then refluxed for 2 hours. Then the reaction mixture was cooled to room temperature and to this mixture 2-fluoroethyl trifluoromethanesulfonate (1.00 g; 4.84 mmol) was added and then stirred at 60° C. overnight. The reaction progress was monitored by TLC. When analysis indicated completion of the reaction, the reaction mixture was cooled to room temperature and the solvent was removed in vacuo and the residue was purified by flash column chromatography on silica (DCM/MeOH, 100:0 to 50:50, v/v, 30 min, 25 mL/min). 1-(2,2-Difluoroethyl)pyrimidine-2,4(1H,3H)-dione was obtained in 96% yield (366 mg; 2.32 mmol).

### Example 102

### Synthesis of 3-((4-(2-((2,7-diazaspiro[3.5]nonan-7-yl)methyl)-5-chloro-3-methylphenyl)pyrrolo[2,1-f][1,2,4]triazin-6-yl)methyl)-6,6-dimethyl-3-azabicyclo[3.1.0]hexane-2,4-dione hydrochloride (102)

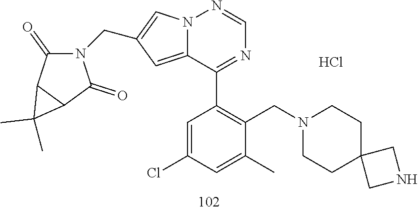 [SMILES: Cc1cc(Cl)cc(-c2ncnn3cc(CN4C(=O)C5C(C4=O)C5(C)C)cc23)c1CN1CCC2(CC1)CNC2 ; Cl]

### Step 1.

### Synthesis of tert-butyl 7-(4-chloro-2-iodo-6-methylbenzyl)-2,7-diazaspiro[3.5]nonane-2-carboxylate (102a)

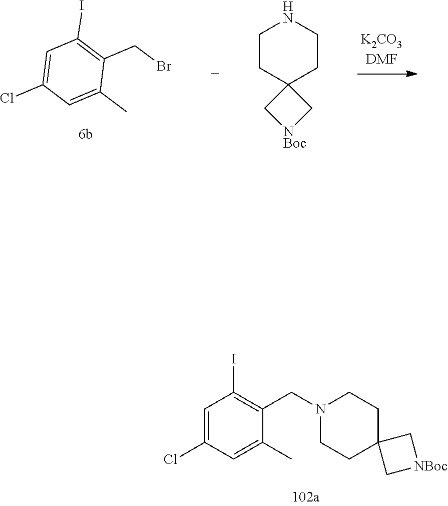 [SMILES: Cc1cc(Cl)cc(I)c1CBr ; CC(C)(C)OC(=O)N1CC2(CCNCC2)C1 ; Cc1cc(Cl)cc(I)c1CN1CCC2(CC1)CN(C(=O)OC(C)(C)C)C2]

Ta a stirred solution of 6b (150 mg; 0.43 mmol) and tert-butyl 2,7-diazaspiro[3.5]nonane-2-carboxylate (98 mg; 0.43 mmol) in DMF (3 mL) K₂CO₃ (89 mg; 0.64 mmol) was added and was stirred under an Argon atmosphere at room temperature overnight. The reaction progress was monitored by LC-MS. When analysis indicated completion of the reaction, the reaction mixture wad diluted with Et₂O, washed with water. The organic layer was dried over anhydrous MgSO₄, filtered and concentrated in vacuo and the residue was purified by flash column chromatography on silica (hexane/AcOEt, 100:0 to 70:30, v/v, 25 min, 13 mL/min). Compound 102a was obtained in 86% yield (180 mg; 0.37 mmol).

### Step 2.

### Synthesis of (2-((2-(tert-butoxycarbonyl)-2,7-diazaspiro[3.5]nonan-7-yl)methyl)-5-chloro-3-methylphenyl)boronic acid (102b)

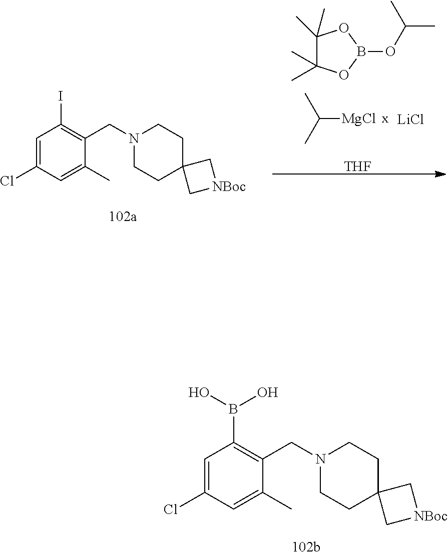

To the solution of 102a (90 mg; 0.183 mmol) in dry THF (1.5 mL) under an Argon atmosphere a isopropyl magnesium chloride lithium chloride complex (1.3 M in THF; 210 μL; 0.274 mmol) was slowly added at −30° C. and stirred for 1 hour in a sealed tube. The temperature was kept between −30° C. and −10° C. Then another portion of isopropyl magnesium chloride lithium chloride complex (1.3 M in THF; 210 μL; 0.274 mmol) was added at −30° C. and stirred for 1 hour in temperature below −10° C. The mixture was recooled to −40° C. and 2-isopropoxy-4,4,5,5-tetramethyl-1,3,2-dioxaborolane (70 μL; 0.366 mmol) was added. The reaction mixture was stirred for 1 hours in the temperature between −30° C. and −10° C. and then at room temperature for 1 hour. The reaction progress was monitored by LC-MS. When analysis indicated completion of the reaction, to the reaction mixture a saturated solution of NH₄Cl (0.5 mL) was added and a whole was stirred for 10 minutes, then a whole was concentrated in vacuo and the residue was purified by preparative reversed-phase column chromatography (column: Cosmosil Cholester 20×250 mm, water+0.3‰ HCl (36%)/MeCN, 90:10 to 30:70, 30 min, 20 mL/min, Rₜ=12.50 min). Compound 102b was obtained in 68% yield (51 mg; 0.125 mmol).

ESI-MS m/z for C₂₀H₃₁BClN₂O₄ found 409.2/411.1 [M+H]⁺; Rₜ=1.29 min (Method A)

### Step 3.

### Synthesis of tert-butyl 7-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylbenzyl)-2,7-diazaspiro[3.5]nonane-2-carboxylate (102c)

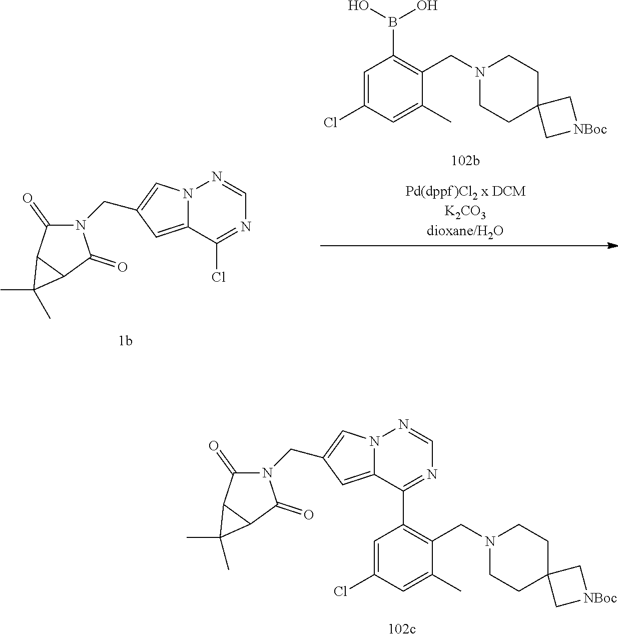 [SMILES: Cc1cc(Cl)cc(B(O)O)c1CN1CCC2(CC1)CN(C(=O)OC(C)(C)C)C2 ; CC1(C)C2C(=O)N(Cc3cc4c(Cl)ncnn4c3)C(=O)C21 ; Cc1cc(Cl)cc(-c2ncnn3cc(CN4C(=O)C5C(C4=O)C5(C)C)cc23)c1CN1CCC2(CC1)CN(C(=O)OC(C)(C)C)C2]

The title compound (102c) was obtained from 1b (37 mg; 0.122 mmol) and from 102b (50 mg; 0.122 mmol) according to the General Procedure IV in 36% yield (28 mg; 0.044 mmol). The crude product was purified by flash column chromatography on silica (hexane/AcOEt, 100:0 to 0:100, v/v, 30 min, 13 mL/min).

ESI-MS m/z for C₃₄H₄₂ClN₆O₄ found 633.2/635.2 [M+H]⁺; Rₜ=1.62 min (Method A)

### Step 4.

### Synthesis of 3-((4-(2-((2,7-diazaspiro[3.5]nonan-7-yl)methyl)-5-chloro-3-methylphenyl)pyrrolo[2,1-f][1,2,4]triazin-6-yl)methyl)-6,6-dimethyl-3-azabicyclo[3.1.0]hexane-2,4-dione hydrochloride (102)

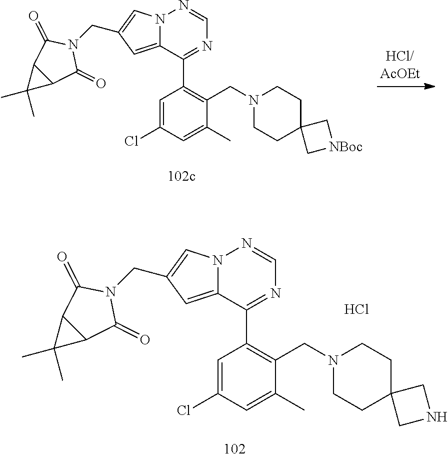 [SMILES: Cc1cc(Cl)cc(-c2ncnn3cc(CN4C(=O)C5C(C4=O)C5(C)C)cc23)c1CN1CCC2(CC1)CN(C(=O)OC(C)(C)C)C2 ; Cc1cc(Cl)cc(-c2ncnn3cc(CN4C(=O)C5C(C4=O)C5(C)C)cc23)c1CN1CCC2(CC1)CNC2 ; Cl]

The title compound (102) was obtained as a hydrochloride salt from 102c (28 mg; 0.044 mmol) according to the General Procedure IIIa in 43% yield (11 mg; 0.019 mmol). The crude product was purified by preparative reversed-phase column chromatography (column: Cosmosil Cholester 20×250 mm, water+0.3‰ HCl (36%)/MeCN, 98:2 to 40:60, 30 min, 20 mL/min, Rₜ=12.50 min).

ESI-MS m/z for C₂₉H₃₄ClN₆O₂ found 533.4/535.4 [M+H]⁺; Rₜ=0.96 min (Method A); ¹H NMR (700 MHz, Methanol-d₄) δ 8.59 (s, 1H), 8.13 (s, 1H), 7.84 (s, 1H), 7.71 (s, 1H), 6.92 (s, 1H), 4.70 (s, 2H), 4.44-4.21 (m, 2H), 4.18-4.01 (m, 2H), 4.01-3.84 (m, 2H), 3.62-3.37 (m, 2H), 2.60 (s, 3H), 2.46 (s, 2H), 2.45-2.27 (m, 2H), 2.24-2.01 (m, 2H), 1.35-1.25 (m, 2H), 1.23 (s, 3H), 1.06 (s, 3H).

### Example 103

### Synthesis of 3-((4-(5-chloro-3-fluoro-2-((4-methylmorpholin-2-yl)methyl)phenyl)pyrrolo[2,1-f][1,2,4]triazin-6-yl)methyl)-1-methylpyrimidine-2,4(1H,3H)-dione hydrochloride (103)

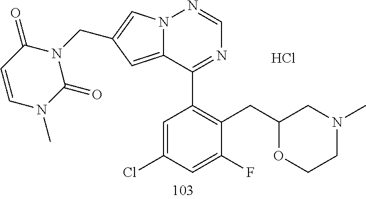 [SMILES: CN1CCOC(Cc2c(F)cc(Cl)cc2-c2ncnn3cc(Cn4c(=O)ccn(C)c4=O)cc23)C1 ; Cl]

### Step 1.

### Synthesis of 4-(4-methoxybenzyl)morpholin-3-one (103a)

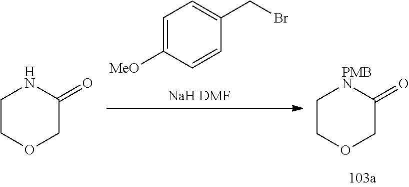

To the cooled to 0° C. solution of morpholin-3-one (1.87 g; 17.90 mmol) in dry DMF (50 mL) NaH (60% in mineral oil; 788 mg; 19.70 mmol) was added under an Argon atmosphere and a whole was then stirred for 10 minutes. Then to this mixture a solution of 1-(bromomethyl)-4-methoxybenzene (3.96 g; 19.70 mmol) in DMF (20 mL) was added dropwise and the reaction mixture was stirred at room temperature overnight. The reaction progress was monitored by LC-MS. When analysis indicated completion of the reaction, the reaction mixture was quenched with 1 M HCl and brine and then extracted with AcOEt (3×). The combined organic layers were washed with water and brine, then dried over anhydrous Na₂SO₄, filtered, and concentrated in vacuo. Then dried under vacuum and Et₂O was added. The solid was filtered off and then dried under vacuum. Compound 103a was obtained in 37% yield (1.48 g; 6.69 mmol).

ESI-MS m/z for C₁₂H₁₆NO₃ found 222.0 [M+H]⁺; Rₜ=0.88 min (Method C); ¹H NMR (400 MHz, DMSO-d₆) δ 7.20-7.15 (m, 2H), 6.91-6.86 (m, 2H), 4.45 (s, 2H), 4.07 (s, 2H), 3.79-3.74 (m, 2H), 3.72 (s, 3H), 3.21-3.17 (m, 2H).

### Step 2.

### Synthesis of 2-(2-bromo-4-chloro-6-fluorobenzyl)-4-(4-methoxybenzyl)morpholin-3-one (103b)

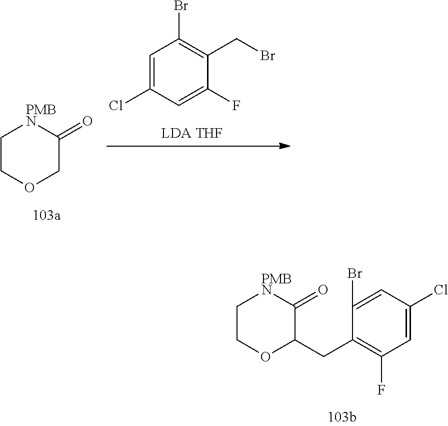

To the cooled to −78° C. solution of 103a (293 mg; 1.32 mmol) in THF (2 mL) LDA (1 M solution in THF/hexanes; 1.59 mL; 1.59 mmol) was added under an Argon atmosphere and a whole was then stirred for 10 minutes at this temperature. Then to this mixture a solution of 1-bromo-2-(bromomethyl)-5-chloro-3-fluorobenzene (0.40 g; 1.32 mmol) in THE (2.2 mL) was added dropwise and the reaction mixture was allowed to warm to room temperature and stirred for 2 hours. The reaction progress was monitored by LC-MS. When analysis indicated completion of the reaction, the reaction mixture was diluted with AcOEt and quenched with a saturated solution of NH₄Cl. An aqueous phase was extracted with AcOEt. The combined organic phases were dried over anhydrous Na₂SO₄, filtered, and concentrated in vacuo. The crude product was purified by flash column chromatography on silica (hexane/AcOEt, 100:0 to 30:70, v/v, 30 min, 20 mL/min). Compound 103b was obtained in 67% yield (392 mg; 0.89 mmol).

ESI-MS m/z for C₁₉H₁₉BrClFNO₃ found 442.0/444.0 [M+H]⁺; Rₜ=1.72 min (Method C); ¹H NMR (400 MHz, CDCl₃) δ 7.43-7.36 (m, 1H), 7.25-7.19 (m, 2H), 7.08-7.02 (m, 1H), 6.90-6.85 (m, 2H), 4.73-4.66 (m, 1H), 4.50-4.39 (m, 2H), 4.01-3.92 (m, 1H), 3.80 (s, 3H), 3.69-3.50 (m, 2H), 3.41-3.33 (m, 1H), 3.28-3.13 (m, 2H).

### Step 3.

### Synthesis of 2-(2-bromo-4-chloro-6-fluorobenzyl)-4-(4-methoxybenzyl)morpholine (103c)

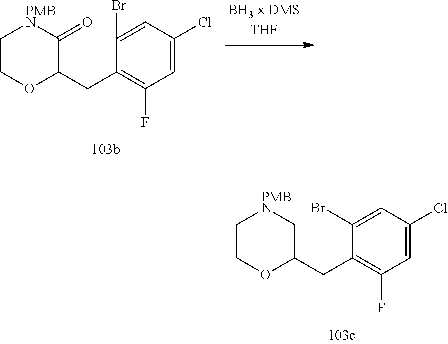

To the cooled to 0° C. solution of 103b (392 mg; 0.89 mmol) in dry THF (3 mL) BH₃×DMS (0.17 mL; 1.77 mmol) was slowly added and this mixture was then stirred at 80° C. for 2 hours. The reaction progress was monitored by LC-MS. When analysis indicated completion of the reaction, the reaction mixture was cooled to 0° C. and to this mixture MeOH was added and a whole was stirred at 80° C. for 1 hour. Then the solvents were removed in vacuo and the residue was purified by flash column chromatography on silica (hexane/AcOEt, 100:0 to 30:70, v/v, 15 min, 30 mL/min). Compound 103c was obtained in 83% yield (315 mg; 0.74 mmol).

ESI-MS m/z for C₁₉H₂₁BrClFNO₂ found 428.0/430.0 [M+H]⁺; Rₜ=1.90 min (Method C)

### Step 4.

### Synthesis of 2-(4-chloro-2-fluoro-6-(4,4,5,5-tetramethyl-1,3,2-dioxaborolan-2-yl)benzyl)-4-(4-methoxybenzyl)morpholine (103d)

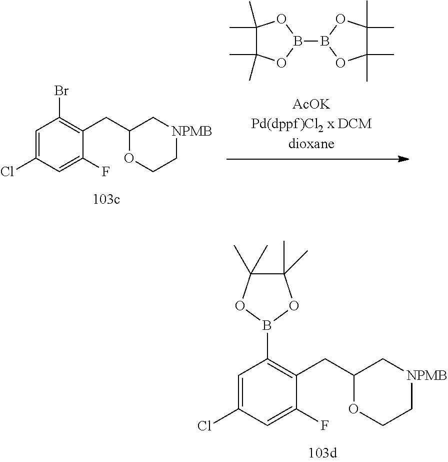

In a Schlenk flask was placed 103c (315 mg; 0.740 mmol), bis(pinacolato)diboron (377 mg; 1.470 mmol), AcOK (219 mg; 2.200 mmol) and Pd(dppf)Cl₂×DCM (31 mg; 0.037 mmol) and dioxane (3.7 mL). The reaction mixture was intensively flushed with Ar. The mixture was capped and stirred at 100° C. overnight. The reaction progress was monitored by LC-MS. When analysis indicated completion of the reaction, the reaction mixture was concentrated in vacuo. The residue was then purified by flash column chromatography on silica (hexane/AcOEt, 100:0 to 0:100, v/v, 30 min, 15 mL/min). Compound 103d was obtained in 99% yield (348 mg; 0.733 mmol).

ESI-MS m/z for C₂₅H₃₃BClFNO₄ found 476.1/478.1 [M+H]⁺; Rₜ=1.54 min (Method C)

### Step 5.

### Synthesis of 3-((4-(5-chloro-3-fluoro-2-((4-(4-methoxybenzyl)morpholin-2-yl)methyl)phenyl)pyrrolo[2,1-f][1,2,4]triazin-6-yl)methyl)pyrimidine-2,4(1H,3H)-dione (103e)

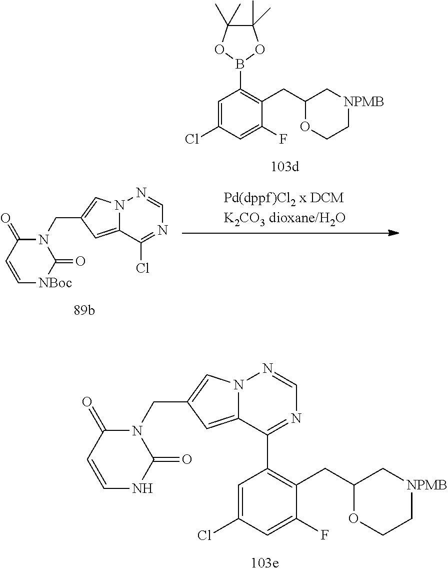

The title compound (103e) was obtained from 89b (278 mg; 0.736 mmol) and from 103d (350 mg; 0.736 mmol) according to the General Procedure IV in 99% yield (430 mg; 0.729 mmol). The crude product was purified by flash column chromatography on silica (DCM/MeOH, 100:0 to 80:20, v/v, 30 min, 20 mL/min).

ESI-MS m/z for C₃₀H₂₉ClFN₆O₄ found 591.3/593.3 [M+H]⁺; Rₜ=1.13 min (Method C)

### Step 6.

### Synthesis of 2-(4-chloro-2-fluoro-6-(6-((3-methyl-2,6-dioxo-3,6-dihydropyrimidin-1(2H)-yl)methyl)pyrrolo[2,1-f][1,2,4]triazin-4-yl)benzyl)-4-(4-methoxybenzyl)-4-methylmorpholin-4-ium (103f)

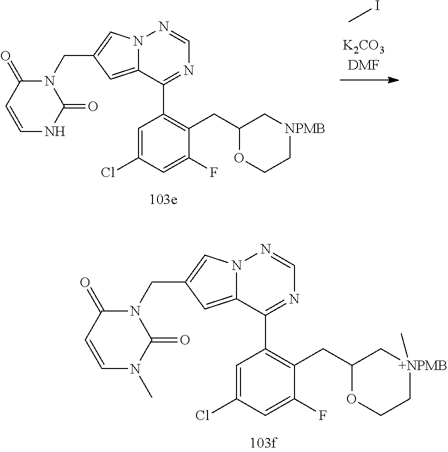

To the solution of 103e (435 mg; 0.736 mmol) in dry DMF (12 mL) K₂CO₃ (208 mg; 1.470 mmol) and methyl iodide (46 μL; 0.736 mmol) were added. The resulting mixture was stirred at room temperature overnight. The reaction progress was monitored by LC-MS. When analysis indicated completion of the reaction, the reaction mixture was diluted with AcOEt and quenched with water (pH=9). An aqueous phase was extracted with AcOEt and neutralized to pH 7 with 1 M HCl, then washed with AcOEt. Then an aqueous layer was acidified to pH 4 with 1 M HCl, extracted with AcOEt and DCM. The combined organic phases were then dried over anhydrous Na₂SO₄, filtered and concentrated in vacuo and the residue was purified by flash column chromatography on silica (DCM/MeOH, 100:0 to 80:20, v/v, 30 min, 20 mL/min). Compound 103f was obtained in 22% yield (100 mg; 0.161 mmol).

ESI-MS m/z for C₃₂H₃₄ClFN₆O₄⁺ found 619.2/621.1 [M]⁺; Rₜ=1.20 min (Method C)

### Step 7.

### Synthesis of 3-((4-(5-chloro-3-fluoro-2-((4-methylmorpholin-2-yl)methyl)phenyl)pyrrolo[2,1-f][1,2,4]triazin-6-yl)methyl)-1-methylpyrimidine-2,4(1H,3H)-dione hydrochloride (103)

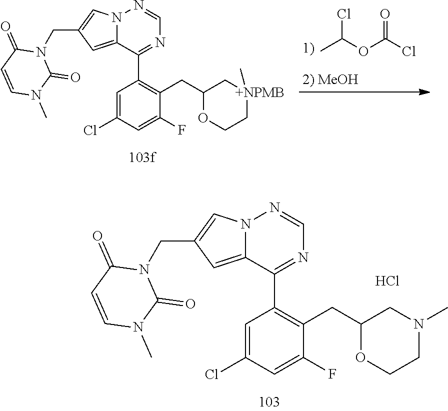

To the solution of 103f (100 mg; 0.161 mmol) in DCE (5.5 mL) 1-chloroethyl carbonochloridate (70 μL; 0.645 mmol) was added dropwise and this mixture was then stirred at 80° C. for 4 hours. The reaction progress was monitored by LC-MS. After additional 2 hours, a second portion of 1-chloroethyl carbonochloridate (107 μL; 0.992 mmol) was added dropwise and this mixture was then stirred at 80° C. for 1 hour. Then another portion of 1-chloroethyl carbonochloridate (1.07 mL; 9.920 mmol) was added dropwise and this mixture was then stirred at 80° C. for 1 week. After this time, the solvents were evaporated in vacuo and the residue was dissolved in MeOH (0.5 mL) and a whole was refluxed for 1 hour. Then the solvent was evaporated in vacuo and the crude product was purified by preparative reversed-phase column chromatography (C-18, water+1‰ HCl (36%)/MeCN, 98:2 to 5:95, 30 min, 20 mL/min, Rₜ=13.87 min). The title compound (103) was obtained as a racemate as a hydrochloride salt in 20% yield (17 mg; 0.032 mmol).

ESI-MS m/z for C₂₄H₂₅ClFN₆O₃ found 499.2/501.2 [M+H]⁺; Rₜ=0.96 min (Method C); ¹H NMR (400 MHz, Methanol-d₄) δ 8.59 (s, 1H), 8.25 (d, J=1.4 Hz, 1H), 7.59-7.52 (m, 2H), 7.52-7.48 (m, 1H), 6.92 (d, J=1.4 Hz, 1H), 5.73 (d, J=7.8 Hz, 1H), 5.23 (s, 2H), 3.87-3.71 (m, 2H), 3.55-3.44 (m, 1H), 3.43-3.39 (m, 1H), 3.38 (s, 3H), 3.29-3.25 (m, 1H), 3.15-2.85 (m, 4H), 2.81 (s, 3H), 2.78-2.67 (m, 1H); ¹⁹F NMR (376 MHz, Methanol-d₄) δ −112.11 (d, J=9.9 Hz).

### Example 104

### Synthesis of (R)-3-((4-(5-chloro-3-methyl-2-(morpholin-2-ylmethyl)phenyl)pyrrolo[2,1-f][1,2,4]triazin-6-yl)methyl)-1-ethylpyrimidine-2,4(1H,3H)-dione hydrochloride (104)

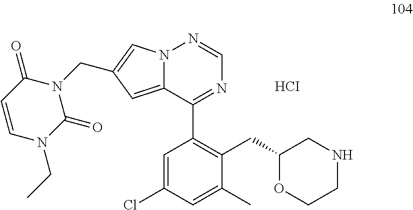

The title compound (104) was obtained as a hydrochloride salt as a single enantiomer (99% ee) in 37% overall yield in a similar way to Example 89 with the exception that, in the fourth step of the synthesis, iodoethane was used instead of 2-iodopropane and in the last step of the synthesis, the crude product was purified by preparative reversed-phase column chromatography (C-18, water+0.3‰ HCl (36%)/MeCN, 98:2 to 30:70, 30 min, 19 mL/min, Rₜ=16.50 min).

ESI-MS m/z for C₂₅H₂₈ClN₆O₃ found 495.4/497.4 [M+H]⁺; Rₜ=1.06 min (Method A); ¹H NMR (700 MHz, Methanol-d₄) δ 8.55 (s, 1H), 8.19 (d, J=1.5 Hz, 1H), 7.60 (d, J=7.8 Hz, 1H), 7.52-7.48 (m, 1H), 7.40-7.36 (m, 1H), 6.82 (d, J=1.5 Hz, 1H), 5.75 (d, J=7.8 Hz, 1H), 5.21 (s, 2H), 3.83 (q, J=7.2 Hz, 2H), 3.78-3.71 (m, 2H), 3.52-3.44 (m, 1H), 3.20-3.15 (m, 1H), 3.10-3.07 (m, 1H), 2.97-2.89 (m, 3H), 2.78-2.71 (m, 1H), 2.48 (s, 3H), 1.27 (t, J=7.2 Hz, 3H).

### Example 105

### Synthesis of (R)-3-((4-(5-chloro-3-methyl-2-(morpholin-2-ylmethyl)phenyl)pyrrolo[2,1-f][1,2,4]triazin-6-yl)methyl)-1-(methyl-d₃)pyrimidine-2,4(1H,3H)-dione hydrochloride (105)

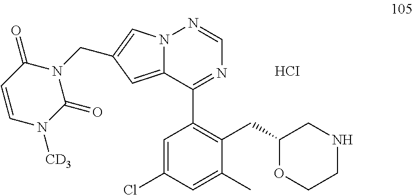

The title compound (105) was obtained as a hydrochloride salt as a single enantiomer (99% ee) in 18% overall yield in a similar way to Example 89 with the exception that, in the fourth step of the synthesis, iodomethane-d₃ was used instead of 2-iodopropane and in the last step of the synthesis, the crude product was purified by preparative reversed-phase column chromatography (C-18, water+0.3‰ HCl (36%)/MeCN, 98:2 to 30:70, 30 min, 20 mL/min, Rₜ=15.10 min).

ESI-MS m/z for C₂₄H₂₃D3ClN₆O₃ found 484.4/486.4 [M+H]⁺; Rₜ=0.98 min (Method A); ¹H NMR (700 MHz, Methanol-d₄) δ 8.52 (s, 1H), 8.14 (d, J=1.5 Hz, 1H), 7.55 (d, J=7.8 Hz, 1H), 7.49 (d, J=2.4 Hz, 1H), 7.37 (d, J=2.3 Hz, 1H), 6.76 (d, J=1.5 Hz, 1H), 5.73 (d, J=7.8 Hz, 1H), 5.21 (s, 2H), 3.77-3.69 (m, 2H), 3.51-3.44 (m, 1H), 3.18-3.12 (m, 1H), 3.12-3.07 (m, 1H), 3.00-2.88 (m, 3H), 2.78-2.72 (m, 1H), 2.48 (s, 3H).

### Example 106

### Synthesis of 3-((4-(5-chloro-3-methyl-2-(((6S)-6-methylmorpholin-2-yl)methyl)phenyl)pyrrolo[2,1-A][1,2,4]triazin-6-yl)methyl)-1-cyclopropylpyrimidine-2,4(1H,3H)-dione hydrochloride (106)

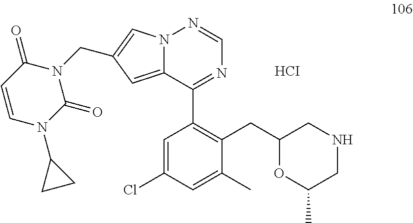

### Step 1.

### Synthesis of tert-butyl (6S)-2-(4-chloro-2-(6-((2,6-dioxo-3,6-dihydropyrimidin-1(2H)-yl)methyl)pyrrolo[2,1-f][1,2,4]triazin-4-yl)-6-methylbenzyl)-6-methylmorpholine-4-carboxylate (106a)

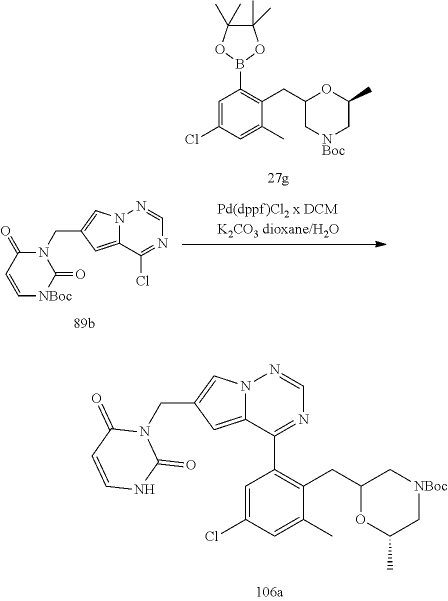 [SMILES: Cc1cc(Cl)cc(B2OC(C)(C)C(C)(C)O2)c1CC1CN(C(=O)OC(C)(C)C)C[C@H](C)O1 ; CC(C)(C)OC(=O)n1ccc(=O)n(Cc2cc3c(Cl)ncnn3c2)c1=O ; Cc1cc(Cl)cc(-c2ncnn3cc(Cn4c(=O)cc[nH]c4=O)cc23)c1CC1CN(C(=O)OC(C)(C)C)C[C@H](C)O1]

The title compound (106a) was obtained from 89b (146 mg; 0.386 mmol) and from 27g (180 mg; 0.386 mmol) according to the General Procedure IV in 7% yield (16 mg; 0.027 mmol). The crude product was purified by flash column chromatography on silica (hexane, 100%, 2 min, then hexane/AcOEt, 100:0 to 0:100, v/v, 8 min, then AcOEt, 100%, 10 min, 15 mL/min).

ESI-MS m/z for C₂₉H₃₄ClN₆O₅ found 581.2/583.1 [M+H]⁺; Rₜ=1.60 min (Method A)

### Step 2.

### Synthesis of tert-butyl (6S)-2-(4-chloro-2-(6-((3-cyclopropyl-2,6-dioxo-3,6-dihydropyrimidin-1(2H)-yl)methyl)pyrrolo[2,1-f][1,2,4]triazin-4-yl)-6-methylbenzyl)-6-methylmorpholine-4-carboxylate (106b)

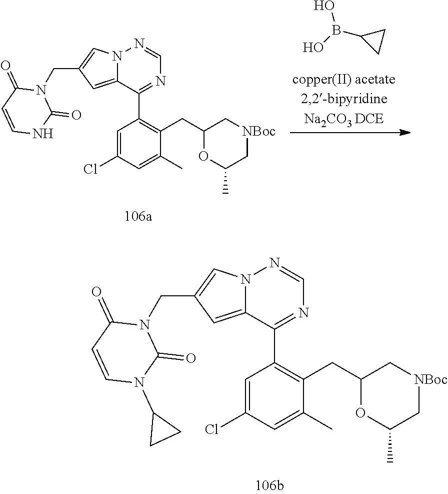 [SMILES: Cc1cc(Cl)cc(-c2ncnn3cc(Cn4c(=O)cc[nH]c4=O)cc23)c1CC1CN(C(=O)OC(C)(C)C)C[C@H](C)O1 ; OB(O)C1CC1 ; Cc1cc(Cl)cc(-c2ncnn3cc(Cn4c(=O)ccn(C5CC5)c4=O)cc23)c1CC1CN(C(=O)OC(C)(C)C)C[C@H](C)O1]

To the solution of 106a (20 mg; 0.034 mmol) and cyclopropylboronic acid (6 mg; 0.069 mmol) in DCE (0.5 mL) copper(II) acetate (6.56 mg; 0.034 mmol), 2,2′-bipyridine (4.4 mg; 0.027 mmol) and Na₂CO₃ (7.3 mg; 0.069 mmol) were added. The resulting mixture was stirred at 70° C. overnight. The reaction progress was monitored by LC-MS. When analysis indicated completion of the reaction, the reaction mixture was diluted with DCM (50 mL) and washed with water (30 mL), 1 M NaOH (30 mL), and brine (30 mL). The organic layer was then dried over anhydrous Na₂SO₄, filtered and concentrated in vacuo and the crude product was used to the next step without any additional purification.

ESI-MS m/z for C₃₂H₃₈ClN₆O₅ found 621.2/623.2 [M+H]⁺; Rₜ=1.79 min (Method A)

### Step 3.

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1-cyclopropylpyrimidine-2,4(1H,3H)-dione hydrochloride (106)

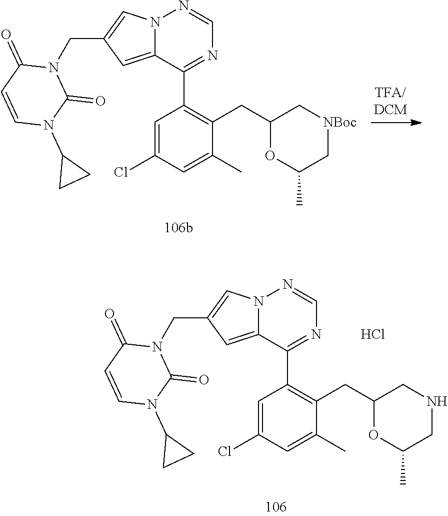 [SMILES: Cc1cc(Cl)cc(-c2ncnn3cc(Cn4c(=O)ccn(C5CC5)c4=O)cc23)c1CC1CN(C(=O)OC(C)(C)C)C[C@H](C)O1 ; Cc1cc(Cl)cc(-c2ncnn3cc(Cn4c(=O)ccn(C5CC5)c4=O)cc23)c1CC1CNC[C@H](C)O1 ; Cl]

The title compound (106) was obtained as a single isomer as a hydrochloride salt from 106b (the crude product) according to the General Procedure IIIb in 35% yield (per two steps)(6.8 mg; 0.012 mmol). The crude product was purified by preparative reversed-phase column chromatography (C-18, water+0.3‰ HCl (36%)/MeCN, 98:2 to 30:70, 30 min, 20 mL/min, Rₜ=17.35 min).

ESI-MS m/z for C₂₇H₃₀ClN₆O₃ found 521.5/523.5 [M+H]⁺; Rₜ=1.12 min (Method A); ¹H NMR (700 MHz, Methanol-d₄) δ 8.63 (s, 1H), 8.36 (d, J=1.5 Hz, 1H), 7.60-7.53 (m, 2H), 7.48 (d, J=2.2 Hz, 1H), 7.05 (s, 1H), 5.71 (d, J=7.9 Hz, 1H), 5.29-5.14 (m, 2H), 3.83-3.76 (m, 1H), 3.58-3.50 (m, 1H), 3.23-3.18 (m, 1H), 3.14-3.03 (m, 3H), 3.00-2.95 (m, 1H), 2.73-2.62 (m, 1H), 2.56-2.42 (m, 4H), 1.05-0.99 (m, 2H), 0.90-0.83 (m, 2H), 0.77 (d, J=6.2 Hz, 3H).

### Example 107

### Synthesis of (R)-3-((4-(5-chloro-3-methyl-2-(morpholin-2-ylmethyl)phenyl)pyrrolo[2,1-f][1,2,4]triazin-6-yl)methyl)-1-cyclopropylpyrimidine-2,4(1H,3H)-dione hydrochloride (107)

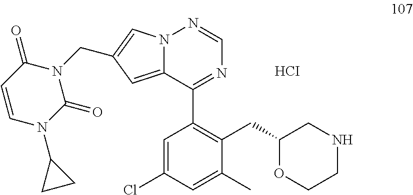

The title compound (107) was obtained as a hydrochloride salt as a single enantiomer (99% ee) in 16% overall yield in a similar way to Example 106 with the exception that, in the first step of the synthesis, the compound 2a (obtained according to the procedure described in the example 82) was used instead of the compound 27g and in the last step of the synthesis, the crude product was purified by preparative reversed-phase column chromatography (C-18, water+0.3‰ HCl (36%)/MeCN, 98:2 to 30:70, 30 min, 20 mL/min, Rₜ=16.50 min).

ESI-MS m/z for C₆₄H₂₈ClN₆O₃ found 507.4/509.4 [M+H]⁺; Rₜ=1.06 min (Method A); ¹H NMR (700 MHz, Methanol-d₄) δ 8.75 (s, 1H), 8.61 (d, J=1.4 Hz, 1H), 7.64 (d, J=2.2 Hz, 1H), 7.59 (d, J=8.0 Hz, 1H), 7.50 (d, J=2.2 Hz, 1H), 7.28 (d, J=1.4 Hz, 1H), 5.72 (d, J=8.0 Hz, 1H), 5.25 (s, 2H), 3.80-3.70 (m, 2H), 3.57-3.50 (m, 1H), 3.30-3.27 (m, 1H), 3.15-3.09 (m, 2H), 3.05-2.98 (m, 1H), 2.95-2.88 (m, 1H), 2.83-2.74 (m, 2H), 2.51 (s, 3H), 1.06-0.99 (m, 2H), 0.89-0.81 (m, 2H).

### Example 108

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-5-fluoro-1-methylpyrimidine-2,4(1H,3H)-dione hydrochloride (108)

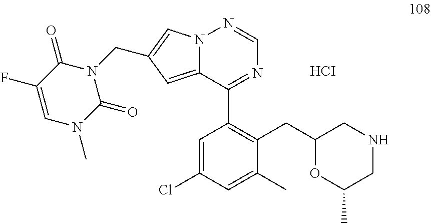

The title compound (108) was obtained as a hydrochloride salt as a single isomer in 2% overall yield in a similar way to Example 89 with the exception that, in the first step of the synthesis, 5-fluorouracil was used instead of pyrimidine-2,4(1H,3H)-dione, in the third step of the synthesis, the compound 27g was used instead of the compound 2a, in the fourth step of the synthesis, methyl iodide was used instead of 2-iodopropane, and in the last step of the synthesis, the crude product was purified by preparative reversed-phase column chromatography (C-18, water+0.3‰ HCl (36%)/MeCN, 98:2 to 30:70, 30 min, 20 mL/min, Rₜ=16.50 min).

ESI-MS m/z for C₂₅H₂₇ClFN₆O₃ found 513.4/515.4 [M+H]⁺; Rₜ=1.08 min (Method A); ¹H NMR (700 MHz, Methanol-d₄) δ 8.67 (d, J=0.9 Hz, 1H), 8.44 (d, J=1.6 Hz, 1H), 7.86 (d, J=6.0 Hz, 1H), 7.59 (d, J=2.2 Hz, 1H), 7.49 (d, J=2.2 Hz, 1H), 7.14 (s, 1H), 5.32-5.20 (m, 2H), 3.84-3.74 (m, 1H), 3.62-3.51 (m, 1H), 3.37 (s, 3H), 3.26-3.20 (m, 1H), 3.10-2.96 (m, 3H), 2.71-2.65 (m, 1H), 2.56-2.46 (m, 4H), 0.77 (d, J=6.2 Hz, 3H).

### Example 109

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1-methyldihydropyrimidine-2,4(1H,3H)-dione hydrochloride (109)

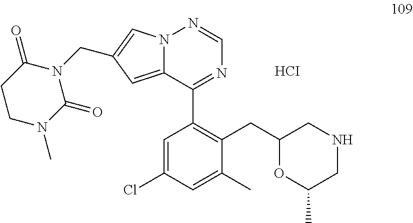

### Step 1.

### Synthesis of (6S)-2-(2-(6-(((tert-butyldimethylsilyl)oxy)methyl)pyrrolo[2,1-f][1,2,4]triazin-4-yl)-4-chloro-6-methylbenzyl)-4-(4-methoxybenzyl)-6-methylmorpholine (109a)

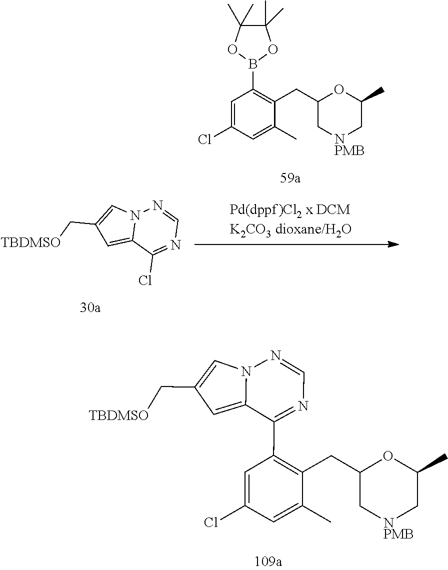

The title compound (109a) was obtained from 30a (44 mg; 0.148 mmol) and from 59a (72 mg; 0.148 mmol) according to the General Procedure IV in 57% yield (53 mg; 0.085 mmol). The crude product was purified by flash column chromatography on silica (hexane/AcOEt, 100:0 to 0:100, v/v, 25 min, 18 mL/min).

ESI-MS m/z for C₃₄H₄₆ClN₄O₃Si found 621.5/623.5 [M+H]⁺; Rₜ=2.13 min (Method A)

### Step 2.

### Synthesis of (4-(5-chloro-2-(((6S)-4-(4-methoxybenzyl)-6-methylmorpholin-2-yl)methyl)-3-methylphenyl)pyrrolo[2,1-f][1,2,4]triazin-6-yl)methanol (109b)

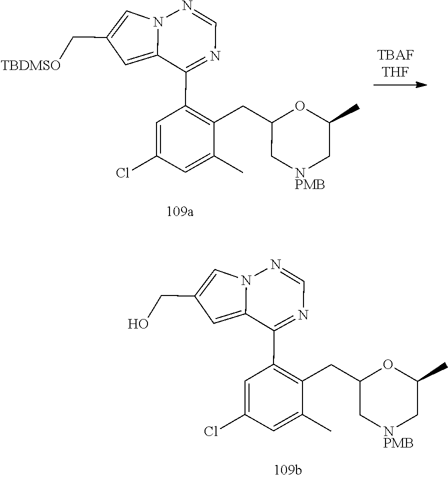

To a solution of 109a (53 mg; 0.085 mmol) in dry THE (0.17 mL) TBAF (1 M in THF; 0.17 mL; 0.171 mmol) was added. The resulting mixture was stirred at room temperature for 1 hour. Then the reaction mixture was concentrated in vacuo and the residue was purified by flash column chromatography on silica (DCM/MeOH, 100:0 to 60:400, v/v, 30 min, 15 mL/min). Compound 109b was obtained in 74% yield (32 mg; 0.063 mmol).

ESI-MS m/z for C₂₈H₃₂ClN₄O₃ found 507.3/509.3 [M+H]⁺; Rₜ=1.31 min (Method A)

### Step 3.

### Synthesis of (6S)-2-(2-(6-(bromomethyl)pyrrolo[2,1-f][1,2,4]triazin-4-yl)-4-chloro-6-methylbenzyl)-4-(4-methoxybenzyl)-6-methylmorpholine (109c)

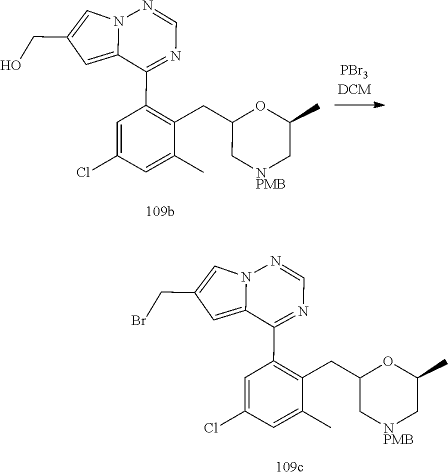

To a cooled to 0° C. solution of 109b (31 mg; 0.061 mmol) in dry DCM (0.2 mL) PBr₃ (11.7 μL; 0.122 mmol) was added dropwise. The resulting mixture was stirred at 0° C. for 1 hour. Then the cooling bath was removed and the reaction mixture was stirred for 3 hours. The reaction progress was monitored by LC-MS. When analysis indicated completion of the reaction, the reaction mixture was cooled to 0° C. and to this mixture 5% NaHCO₃ was carefully added and a whole was stirred for 10 minutes. The layers were separated and an organic one was washed with 5% NaHCO₃, dried over anhydrous Na₂SO₄, filtered and concentrated in vacuo. Compound 109c was obtained in 87% yield (30 mg; 0.053 mmol).

ESI-MS m/z for C₂₈H₃₁BrClN₄O₂ found 569.1/571.0 [M+H]⁺; Rₜ=1.77 min (Method A)

### Step 4.

### Synthesis of 3-((4-(5-chloro-2-(((6S)-4-(4-methoxybenzyl)-6-methylmorpholin-2-yl)methyl)-3-methylphenyl)pyrrolo[2,1-f][1,2,4]triazin-6-yl)methyl)-1-methyldihydropyrimidine-2,4(1H,3H)-dione (109d)

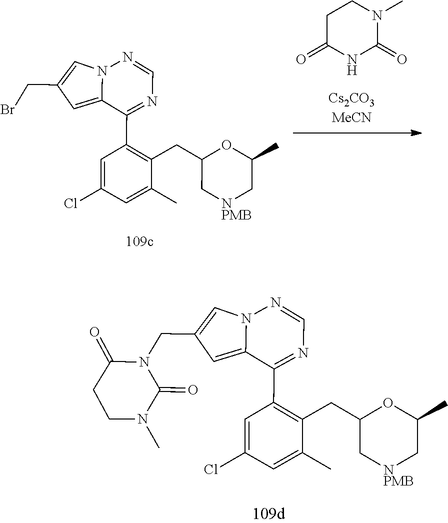

To the solution of 109c (22 mg; 0.039 mmol) in MeCN (0.3 mL) 1-methyldihydropyrimidine-2,4(1H,3H)-dione (5.2 mg; 0.039 mmol) and Cs₂CO₃ (38 mg; 0.116 mmol) were added and a whole was stirred under argon atmosphere at room temperature for 4 hours. The reaction progress was monitored by LC-MS. When analysis indicated completion of the reaction, the solvent was evaporated in vacuo and to the residue water and AcOEt were added. An aqueous layer was extracted with AcOEt (2×). The combined organic solutions were dried over anhydrous Na₂SO₄, filtered and concentrated in vacuo and the residue was purified by flash column chromatography on silica (DCM/MeOH, 100:0 to 0:100, v/v, 11 min, 18 mL/min). Compound 109d was obtained in 82% yield (20 mg; 0.032 mmol).

ESI-MS m/z for C₃₃H₃₈ClN₆O₄ found 617.4/619.4 [M+H]⁺; Rₜ=1.40 min (Method A)

### Step 5.

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1-methyldihydropyrimidine-2,4(1H,3H)-dione hydrochloride (109)

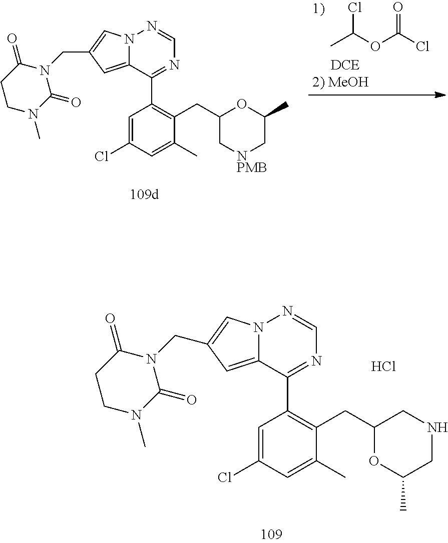

To the cooled to 0° C. solution of 109d (19 mg; 0.031 mmol) in DCE (0.12 mL) 1-chloroethyl carbonochloridate (13 μL; 0.123 mmol) was added dropwise and this mixture was then stirred at 80° C. for 4 hours. The reaction progress was monitored by LC-MS. When the intermediate was formed the solvent was evaporated in vacuo and the residue was dissolved in MeOH (0.32 mL) and a whole was stirred at 80° C. for 1 hour. Then the solvent was evaporated in vacuo and the crude product was purified by preparative reversed-phase column chromatography (column: Cosmosil Cholester 20×250 mm, water+0.3‰ HCl (36%)/MeCN, 99:1 to 20:80, 30 min, 20 mL/min, Rₜ=14.60 min). The title compound (109) was obtained as a hydrochloride salt in 10% yield (1.4 mg; 0.003 mmol).

ESI-MS m/z for C₂₅H₃₀ClN₆O₃ found 497.4/499.4 [M+H]⁺; Rₜ=1.08 min (Method A); ¹H NMR (700 MHz, Methanol-d₄) δ 8.51 (s, 1H), 8.09 (s, 1H), 7.50 (d, J=2.0 Hz, 1H), 7.42 (d, J=2.1 Hz, 1H), 6.76 (s, 1H), 5.04-4.98 (m, 2H), 3.80-3.76 (m, 1H), 3.54-3.50 (m, 1H), 3.41 (t, J=6.9 Hz, 2H), 3.17-3.12 (m, 2H), 3.09-3.06 (m, 1H), 3.01 (s, 3H), 2.94-2.91 (m, 1H), 2.74 (t, J=6.9 Hz, 2H), 2.68-2.64 (m, 1H), 2.55-2.51 (m, 1H), 2.48 (s, 3H), 0.81 (d, J=6.2 Hz, 3H).

### Example 110

### Synthesis of 3-((4-(5-chloro-3-methyl-2-(((5S)-5-methylmorpholin-2-yl)methyl)phenyl)pyrrolo[2,1-f][1,2,4]triazin-6-yl)methyl)-1-methylpyrimidine-2,4(1H,3H)-dione hydrochloride (110)

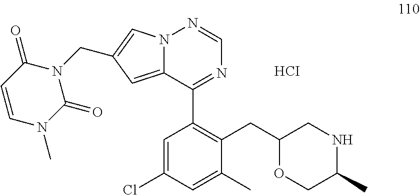

### Step 1.

### Synthesis of (S)-2-((4-methoxybenzyl)amino)propan-1-ol (110a)

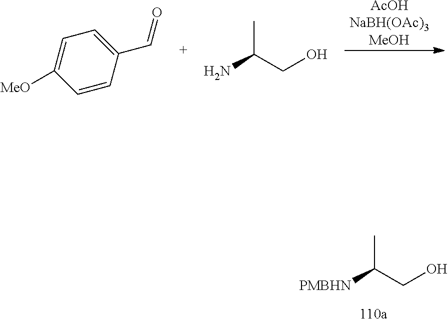

To the solution of (S)-2-aminopropan-1-ol (1.00 g; 13.20 mmol) in MeOH (30 mL) 4-methoxybenzaldehyde (1.60 mL; 13.20 mmol) and glacial acetic acid (AcOH) (0.67 mL; 11.50 mmol) were added and the mixture was stirred for 40 minutes at room temperature. Then sodium triacetoxyborohydride (NaBH(OAc)₃; 3.08 g; 14.50 mmol) was then added in several portions and this mixture was stirred at room temperature overnight. After this time MeOH was evaporated in vacuo. The crude product was purified by flash column chromatography on silica (DCM, 100%, 5 min, then: DCM/MeOH, 100:0 to 40:60, v/v, 15 min, 25 mL/min). Compound 110a was obtained in 99% yield (2.55 g; 13.07 mmol).

ESI-MS m/z for C₁₁H₁₈NO₂ found 196.1 [M+H]⁺; Rₜ=0.25 min (Method A)

### Step 2.

### Synthesis of (S)-4-(4-methoxybenzyl)-5-methylmorpholin-3-one (110b)

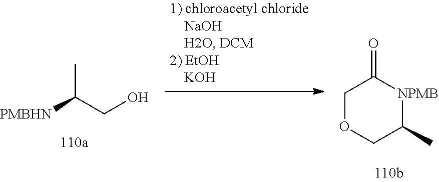

To the solution of 110a (2.55 g; 13.07 mmol) in DCM (15 mL) a solution of NaOH (528 mg; 13.20 mmol) in H₂O (10 mL) was added and the mixture was cooled to 0° C. Then to this solution chloroacetyl chloride (1.07 mL; 13.20 mmol) was added dropwise and the reaction mixture was then stirred at room temperature overnight. Then another portion of chloroacetyl chloride (0.53 mL; 6.60 mmol) was added dropwise and stirred at room temperature. After this time, the phases were separated and an organic one was washed with 1 M NaOH, 1 M HCl and brine and then was dried over anhydrous Na₂SO₄, filtered and concentrated in vacuo. The residue was dissolved in EtOH (15 mL) and to this solution KOH (473 mg; 8.43 mmol) was added. The reaction mixture was stirred at room temperature overnight. Then the crude product was purified by flash column chromatography on silica (hexane, 100%, 3 min, then: hexane/AcOEt, 100:0 to 0:100, v/v, 12 min, 25 mL/min). Compound 110b was obtained in 30% yield (per two steps)(930 mg; 3.95 mmol).

ESI-MS m/z for C₁₃H₁₈NO₃ found 236.0 [M+H]⁺; Rₜ=0.94 min (Method A); ¹H NMR (700 MHz, CDCl₃) δ 7.21-7.17 (m, 2H), 6.87-6.84 (m, 2H), 5.36-5.29 (m, 1H), 4.30-4.19 (m, 2H), 3.92-3.86 (m, 1H), 3.80 (s, 3H), 3.74-3.69 (m, 1H), 3.66-3.60 (m, 1H), 3.37-3.32 (m, 1H), 1.29-1.26 (m, 3H).

### Step 3.

### Synthesis of (5S)-2-(4-chloro-2-iodo-6-methylbenzyl)-4-(4-methoxybenzyl)-5-methylmorpholin-3-one (110c)

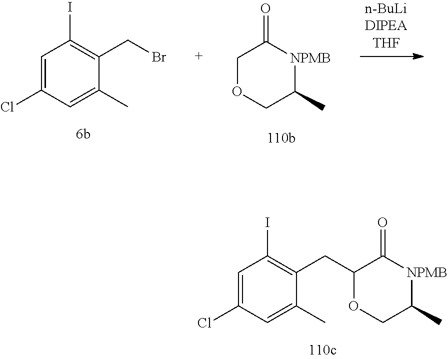

To the cooled to −78° C. solution of DIPEA (609 μL; 4.35 mmol) in dry THF (14 mL) n-BuLi (2.5 M in hexane; 1.74 mL; 4.35 mmol) was added dropwise and the resulting mixture was warmed to room temperature and stirred at this temperature for 10 minutes. Then the reaction mixture was cooled to −78° C. and to this mixture a solution of 110b (930 mg; 3.95 mmol) in dry THE (4 mL) was added and the mixture was stirred at this temperature for 1 hour. Then to this mixture a solution of 6b (1.36 g; 3.95 mmol) in THE (4 mL) was added and a whole was allowed to warm to room temperature for 1 hour. After this time, the reaction mixture was quenched with NH₄Cl (50 mL) and extracted with AcOEt (2×30 mL). The combined organic solutions were dried over anhydrous Na₂SO₄, filtered and concentrated in vacuo. Then the crude product was purified by flash column chromatography on silica (hexane, 100%, 4 min, then: hexane/AcOEt, 100:0 to 80:20, v/v, 16 min, 25 mL/min). Compound 110c was obtained in 43% yield (850 mg; 1.70 mmol).

ESI-MS m/z for C₂₁H₂₄ClINO₃ found 500.0/501.9 [M+H]⁺; Rₜ=1.90 min (Method A); ¹H NMR (700 MHz, CDCl₃) δ 7.74-7.71 (m, 1H), 7.25-7.20 (m, 2H), 7.17-7.14 (m, 1H), 6.91-6.85 (m, 2H), 5.38-5.25 (m, 1H), 4.50-4.41 (m, 1H), 4.04-3.84 (m, 2H), 3.83-3.79 (m, 3H), 3.65-3.42 (m, 3H), 3.31-3.21 (m, 1H), 2.47-2.38 (m, 3H), 1.41-1.16 (m, 3H).

### Step 4.

### Synthesis of (5S)-2-(4-chloro-2-iodo-6-methylbenzyl)-4-(4-methoxybenzyl)-5-methylmorpholine (110d and 110d′)

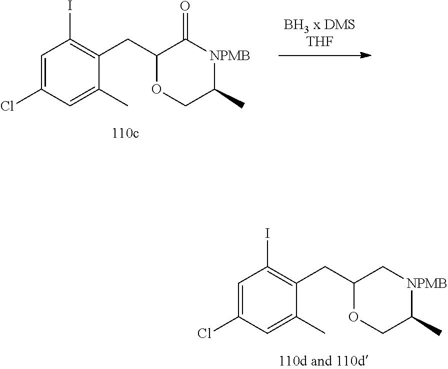

To the cooled to 0° C. solution of 110c (850 mg; 1.70 mmol) in dry THF (20 mL) BH₃×DMS (0.33 mL; 3.40 mmol) was added under an Argon atmosphere and this mixture was then stirred at 65° C. for 3 hours. Then to the reaction mixture 6 M HCl (5 mL) was added and a whole was stirred at 65° C. for 1 hour. The reaction progress was monitored by LC-MS. When analysis indicated completion of the reaction, the reaction mixture was cooled to room temperature and basified with 4 M NaOH, then extracted with AcOEt (2×30 mL). The combined organic extracts were washed with water (30 mL), dried over anhydrous Na₂SO₄, filtered and concentrated in vacuo. The crude product was purified by flash column chromatography on silica (hexane, 100%, 3 min, then: hexane/AcOEt, 100:0 to 70:30, v/v, 12 min, then: hexane/AcOEt, 70:30, v/v, 5 min, 20 mL/min). Compounds 110d and 110d′ were obtained as two separated diastereoisomers in 30% yield (for 110d)(250 mg; 0.51 mmol) and in 55% yield (for 110d′)(450 mg; 0.93 mmol).

For 110d: ESI-MS m/z for C₂₁H₂₆ClINO₂ found 486.1/487.9 [M+H]⁺; Rₜ=1.49 min (Method A); ¹H NMR (700 MHz, CDCl₃) δ 7.71-7.65 (m, 1H), 7.27-7.22 (m, 2H), 7.14-7.08 (m, 1H), 6.86-6.82 (m, 2H), 3.84-3.77 (m, 4H), 3.67-3.62 (m, 1H), 3.59-3.47 (m, 3H), 3.06-3.00 (m, 1H), 2.87-2.80 (m, 1H), 2.72-2.66 (m, 1H), 2.57-2.45 (m, 2H), 2.38 (s, 3H), 1.08 (d, J=6.6 Hz, 3H).

For 110d′: ESI-MS m/z for C₂₁H₂₆ClINO₂ found 486.2/487.9 [M+H]⁺; Rₜ=1.46 min (Method A); ¹H NMR (700 MHz, CDCl₃) δ 7.65 (d, J=2.2 Hz, 1H), 7.24-7.19 (m, 2H), 7.09 (d, J=2.2 Hz, 1H), 6.88-6.83 (m, 2H), 4.02-3.94 (m, 1H), 3.81 (s, 3H), 3.76-3.70 (m, 1H), 3.68-3.63 (m, 1H), 3.24-3.16 (m, 2H), 2.96-2.91 (m, 1H), 2.75-2.70 (m, 2H), 2.41-2.37 (m, 1H), 2.34 (s, 3H), 2.05-2.00 (m, 2H), 1.05 (d, J=6.2 Hz, 3H).

### Step 5.

### Synthesis of (5S)-2-(4-chloro-2-methyl-6-(4,4,5,5-tetramethyl-1,3,2-dioxaborolan-2-yl)benzyl)-4-(4-methoxybenzyl)-5-methylmorpholine (110e)

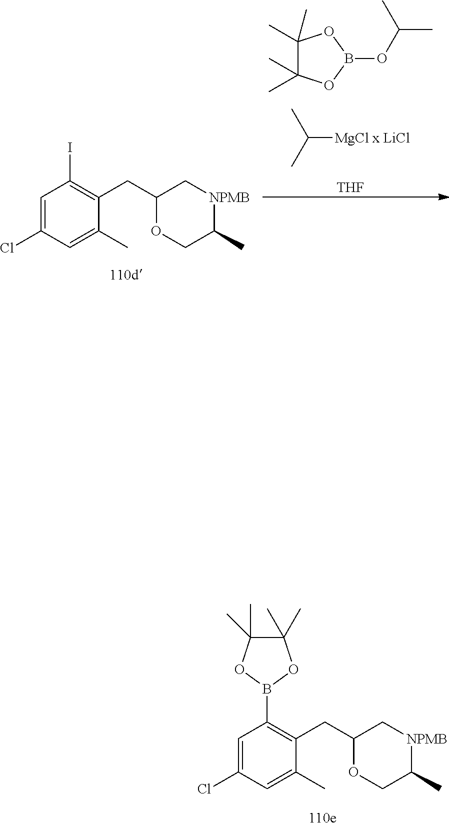

To the cooled to −10° C. solution of 110d′ (425 mg; 0.875 mmol) in dry THF (5 mL) a isopropyl magnesium chloride lithium chloride complex (1.3 M in THF; 1.35 mL; 1.750 mmol) was added dropwise and after stirring in this temperature for 20 minutes 2-isopropoxy-4,4,5,5-tetramethyl-1,3,2-dioxaborolane (461 μL; 2.190 mmol) was added and then the reaction mixture was allowed to warm to room temperature and stirred for 1 hour. The reaction progress was monitored by LC-MS. When analysis indicated completion of the reaction, the reaction mixture was quenched with an aqueous saturated solution of NH₄Cl (40 mL) and a whole was extracted with AcOEt (2×50 mL). The combined organic layers were dried over anhydrous Na₂SO₄, filtered and concentrated in vacuo. The crude product was used to the next step without any additional purification.

ESI-MS m/z for C₂₇H₃₈BClNO₄ found 485.9/487.9 [M+H]⁺; Rₜ=1.66 min (Method A)

### Step 6.

### Synthesis of 3-((4-(5-chloro-2-(((5S)-4-(4-methoxybenzyl)-5-methylmorpholin-2-yl)methyl)-3-methylphenyl)pyrrolo[2,1-f][1,2,4]triazin-6-yl)methyl)pyrimidine-2,4(1H,3H)-dione (110f)

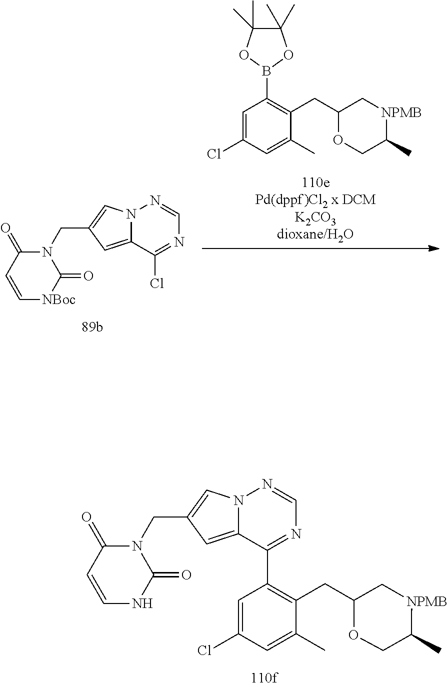

The title compound (110f) was obtained from 89b (326 mg; 0.864 mmol) and from 110e (the crude product) according to the General Procedure IV in 20% yield (per two steps)(103 mg; 0.172 mmol). The crude product was purified by flash column chromatography on silica (hexane, 100%, 2 min, then: hexane/AcOEt, 100:0 to 0:100, v/v, 8 min, then AcOEt, 100%, 20 min, 30 mL/min).

ESI-MS m/z for C₃₂H₃₄ClN₆O₄ found 601.2/603.2 [M+H]⁺; Rₜ=1.21 min (Method A)

### Step 7.

### Synthesis of 3-((4-(5-chloro-3-methyl-2-(((5S)-5-methylmorpholin-2-yl)methyl)phenyl)pyrrolo[2,1-f][1,2,4]triazin-6-yl)methyl)pyrimidine-2,4(1H,3H)-dione (110g)

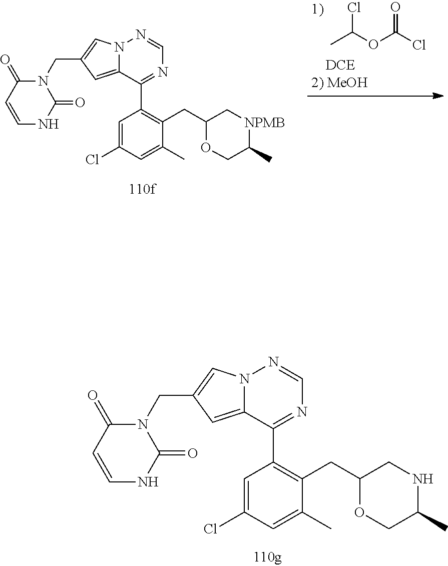

To the cooled to 0° C. solution of 110f (100 mg; 0.166 mmol) in DCE (1 mL) 1-chloroethyl carbonochloridate (72 μL; 0.664 mmol) was added dropwise and this mixture was then stirred at 80° C. overnight. Then during 3 days an additional portion of 1-chloroethyl carbonochloridate (215 μL; 1.990 mmol) was added dropwise and this mixture was then stirred at 80° C. for 4 days. The reaction progress was monitored by LC-MS. When analysis indicated completion of the reaction, the solvent was evaporated in vacuo and the residue was dissolved in anhydrous MeOH (5 mL) and a whole was stirred at 80° C. for 1 hour. Then the solvent was removed in vacuo and the residue was purified by flash column chromatography on silica (DCM, 100%, 3 min, then: DCM/MeOH, 100:0 to 0:100, v/v, 8 min, 15 mL/min). Compound 110g was obtained in 33% yield (53 mg; 0.110 mmol; 50% of purity).

ESI-MS m/z for C₂₄H₂₆ClN₆O₃ found 481.0/483.0 [M+H]⁺; Rₜ=1.01 min (Method A)

### Step 8.

### Synthesis of tert-butyl (5S)-2-(4-chloro-2-(6-((2,6-dioxo-3,6-dihydropyrimidin-1(2H)-yl)methyl)pyrrolo[2,1-f][1,2,4]triazin-4-yl)-6-methylbenzyl)-5-methylmorpholine-4-carboxylate (110h)

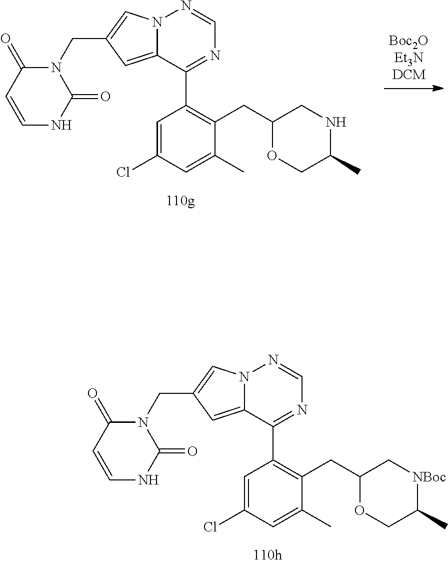 [SMILES: Cc1cc(Cl)cc(-c2ncnn3cc(Cn4c(=O)cc[nH]c4=O)cc23)c1CC1CN[C@@H](C)CO1 ; Cc1cc(Cl)cc(-c2ncnn3cc(Cn4c(=O)cc[nH]c4=O)cc23)c1CC1CN(C(=O)OC(C)(C)C)[C@@H](C)CO1]

To the solution of 110g (26 mg; 0.054 mmol) in DCM (5 mL) Boc₂O (12 mg; 0.054 mmol) and Et₃N (0.01 mL; 0.081 mmol) were added and this mixture was then stirred at room temperature for 2 hours. The reaction progress was monitored by LC-MS. When analysis indicated completion of the reaction, the solvent was evaporated in vacuo. The crude product was used to the next step without any additional purification.

ESI-MS m/z for C₂₉H₃₄ClN₆O₅ found 581.2/583.3 [M+H]⁻; Rₜ=1.50 min (Method A)

### Step 9.

### Synthesis of tert-butyl (5S)-2-(4-chloro-2-methyl-6-(6-((3-methyl-2,6-dioxo-3,6-dihydropyrimidin-1(2H)-yl)methyl)pyrrolo[2,1-f][1,2,4]triazin-4-yl)benzyl)-5-methylmorpholine-4-carboxylate (110i)

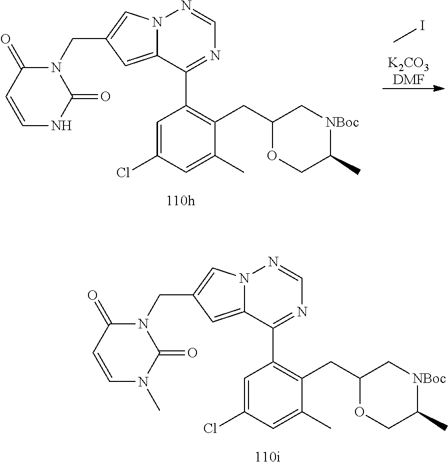

To the solution of 110h (the crude product) in dry DMF (1 mL) K₂CO₃ (5 mg; 0.035 mmol) and methyl iodide (1.6 μL; 0.026 mmol) were added. The resulting mixture was stirred at room temperature overnight. The reaction progress was monitored by LC-MS. When analysis indicated completion of the reaction, the reaction mixture was concentrated in vacuo and the residue was purified by flash column chromatography on silica (hexane, 100%, 1 min, then hexane/AcOEt, 100:0 to 0:100, v/v, 5 min, then AcOEt, 100%, 9 min, 15 mL/min). Compound 110i was obtained in 19% yield (per two steps)(17 mg; 0.029 mmol; 36% of purity).

ESI-MS m/z for C₃₀H₃₆ClN₆O₅ found 595.1/597.1 [M+H]⁺; Rₜ=1.58 min (Method A)

### Step 10.

### Synthesis of 3-((4-(5-chloro-3-methyl-2-(((5S)-5-methylmorpholin-2-yl)methyl)phenyl)pyrrolo[2,1-f][1,2,4]triazin-6-yl)methyl)-1-methylpyrimidine-2,4(1H,3H)-dione hydrochloride (110)

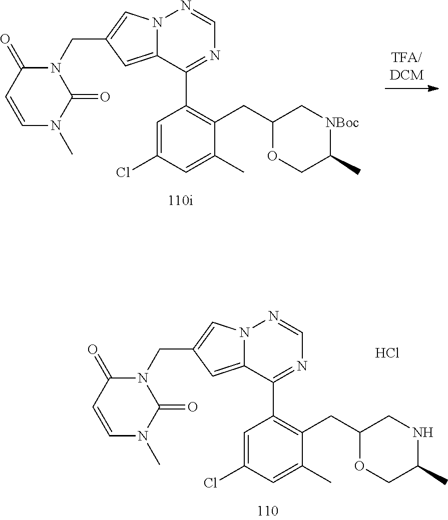 [SMILES: Cc1cc(Cl)cc(-c2ncnn3cc(Cn4c(=O)ccn(C)c4=O)cc23)c1CC1CN(C(=O)OC(C)(C)C)[C@@H](C)CO1 ; Cc1cc(Cl)cc(-c2ncnn3cc(Cn4c(=O)ccn(C)c4=O)cc23)c1CC1CN[C@@H](C)CO1 ; Cl]

The title compound (110) was obtained as a single isomer as a hydrochloride salt from 110i (17 mg; 0.010 mmol) according to the General Procedure IIIb in 60% yield (3.3 mg; 0.006 mmol). The crude product was purified by preparative reversed-phase column chromatography (C-18, water+0.3‰ HCl (36%)/MeCN, 98:2 to 30:70, 30 min, 20 mL/min, Rₜ=15.25 min).

ESI-MS m/z for C₂₅H₂₈ClN₆O₃ found 495.4/497.4 [M+H]⁺; Rₜ=1.00 min (Method A); ¹H NMR (700 MHz, Methanol-d₄) δ 8.62 (s, 1H), 8.38 (d, J=1.5 Hz, 1H), 7.61-7.51 (m, 2H), 7.44 (d, J=2.6 Hz, 1H), 7.02 (d, J=1.5 Hz, 1H), 5.73 (d, J=7.8 Hz, 1H), 5.24 (s, 2H), 3.71-3.63 (m, 2H), 3.37 (s, 3H), 3.28-3.24 (m, 1H), 3.20-3.14 (m, 1H), 3.14-3.09 (m, 1H), 3.01-2.97 (m, 1H), 2.90-2.85 (m, 1H), 2.82-2.78 (m, 1H), 2.49 (s, 3H), 1.13 (d, J=6.4 Hz, 3H).

### Example 111

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1-isopropylpyrimidine-2,4(1H,3H)-dione hydrochloride (111)

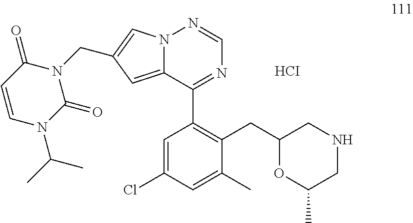

The title compound (111) was obtained as a hydrochloride salt in 1% overall yield in a similar way to Example 110 with the exception that, in the first step of the synthesis, (S)-1-aminopropan-2-ol was used instead of (S)-2-aminopropan-1-ol, in the ninth step of the synthesis, 2-iodopropane (stabilized with copper) was used instead of methyl iodide, and in the last step of the synthesis, the crude product was purified by preparative reversed-phase column chromatography (C-18, water+0.3‰ HCl (36%)/MeCN, 99:1 to 20:80, 30 min, 20 mL/min, Rₜ=16.16 min).

ESI-MS m/z for C₂₇H₃₂ClN₆O₃ found 523.5/525.5 [M+H]⁺; Rₜ=1.13 min (Method A); ¹H NMR (700 MHz, Methanol-d₄) δ 8.73 (s, 1H), 8.53 (d, J=1.2 Hz, 1H), 7.69 (d, J=8.0 Hz, 1H), 7.63 (d, J=2.0 Hz, 1H), 7.52 (d, J=2.1 Hz, 1H), 7.24 (s, 1H), 5.80 (d, J=8.0 Hz, 1H), 5.29-5.22 (m, 2H), 4.82-4.77 (m, 1H), 3.85-3.80 (m, 1H), 3.61-3.55 (m, 1H), 3.29-3.24 (m, 1H), 3.11-3.07 (m, 1H), 3.04-2.97 (m, 2H), 2.71-2.66 (m, 1H), 2.54-2.49 (m, 4H), 1.34 (s, 3H), 1.33 (s, 3H), 0.76 (d, J=6.2 Hz, 3H).

### Example 112

### Synthesis of 3-((4-(5-chloro-2-((S)-3-(methylamino)piperidin-1-yl)phenyl)pyrrolo[2,1-f][1,2,4]triazin-6-yl)methyl)-6,6-dimethyl-3-azabicyclo[3.1.0]hexane-2,4-dione hydrochloride (112)

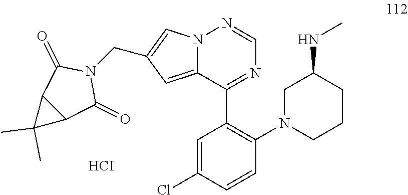

### Synthesis of (S)-1-(2-bromo-4-chlorophenyl)piperidin-3-amine (112a)

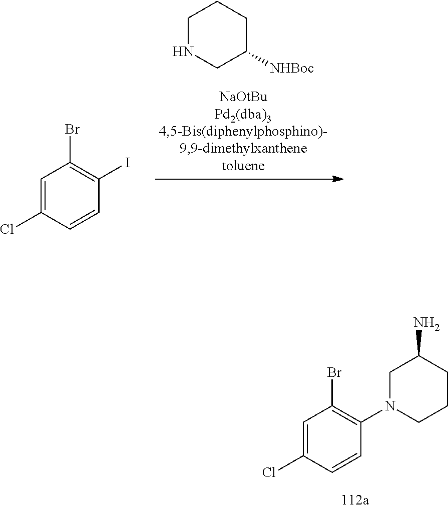 [SMILES: CC(C)(C)OC(=O)N[C@H]1CCCNC1 ; Clc1ccc(I)c(Br)c1 ; N[C@H]1CCCN(c2ccc(Cl)cc2Br)C1]

To the solution of 2-bromo-4-chloro-1-iodobenzene (500 mg; 1.580 mmol), tert-butyl (S)-piperidin-3-ylcarbamate (325 mg; 1.580 mmol) and 4,5-Bis(diphenylphosphino)-9,9-dimethylxanthene (91 mg; 0.158 mmol) in dry toluene (6 mL) NaOtBu (309 mg; 3.150 mmol) was added and the suspension was intensively flushed with Ar. Then to this suspension tris(dibenzylideneacetone)dipalladium(0) (Pd₂(dba)₃; 149 mg; 0.158 mmol) was added and a whole was stirred at 100° C. overnight in a sealed tube. The reaction progress was monitored by LC-MS. The reaction mixture was concentrated in vacuo and the residue was used to the next step without any additional purification.

ESI-MS m/z for C₁₁H₁₅BrClN₂ found 288.9/290.9 [M+H]⁺; Rₜ=1.00 min (Method A)

### Step 2.

### Synthesis of tert-butyl (S)-(1-(2-bromo-4-chlorophenyl)piperidin-3-yl)carbamate (112b)

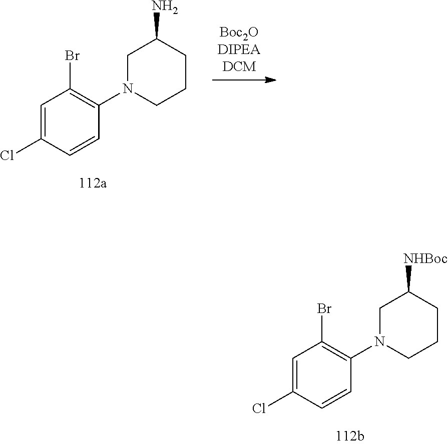 [SMILES: N[C@H]1CCCN(c2ccc(Cl)cc2Br)C1 ; CC(C)(C)OC(=O)N[C@H]1CCCN(c2ccc(Cl)cc2Br)C1]

To the solution of 112a (the crude product) in dry DCM (10 mL) Boc₂O (500 mg; 2.29 mmol) and DIPEA (0.50 mL; 2.91 mmol) were added and this mixture was then stirred at room temperature overnight. The reaction progress was monitored by LC-MS. Then the reaction mixture was diluted with DCM and washed with 1 M HCl. The organic layer was dried over anhydrous Na₂SO₄, filtered and concentrated in vacuo. The crude product was purified by flash column chromatography on silica (hexane/AcOEt, 100:0 to 85:15, v/v, 30 min, 30 mL/min). Compound 112b was obtained in 11% yield (per two steps)(70 mg; 0.18 mmol).

ESI-MS m/z for C₁₆H₂₃BrClN₂O₂ found 388.9/390.6 [M+H]⁺; Rₜ=1.92 min (Method A)

### Step 3.

### Synthesis of tert-butyl (S)-(1-(2-bromo-4-chlorophenyl)piperidin-3-yl)(methyl)carbamate (112c)

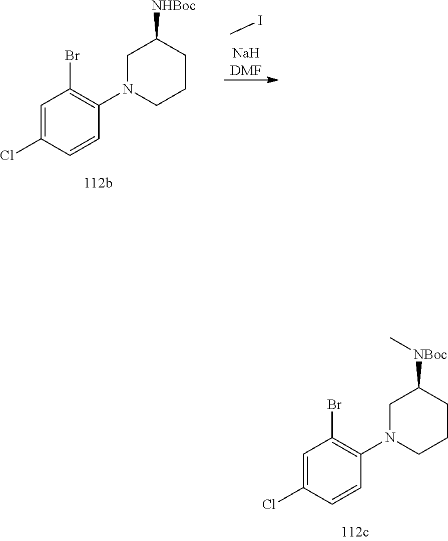

To the solution of 112b (70 mg; 0.180 mmol) in dry DMF (1.5 mL) NaH (60% in mineral oil; 14.4 mg; 0.359 mmol) was added and the suspension was stirred at room temperature for 30 minutes. Then to this suspension methyl iodide (16.9 μL; 0.269 mmol) was added and a whole was stirred at room temperature overnight. The reaction progress was monitored by LC-MS. When analysis indicated completion of the reaction, to the reaction mixture water was carefully added and the product was extracted with AcOEt. The organic layer was dried over anhydrous Na₂SO₄, filtered and concentrated in vacuo. The crude product was purified by flash column chromatography on silica (hexane/AcOEt, 100:0 to 80:20, v/v, 20 min, 13 mL/min). Compound 112c was obtained in 93% yield (67 mg; 0.167 mmol).

ESI-MS m/z for C₁₇H₂₅BrClN₂O₂ found 402.9/404.9 [M+H]⁺; Rₜ=2.10 min (Method A)

### Step 4.

### Synthesis of (S)-(2-(3-((tert-butoxycarbonyl)(methyl)amino)piperidin-1-yl)-5-chlorophenyl)boronic acid (112d)

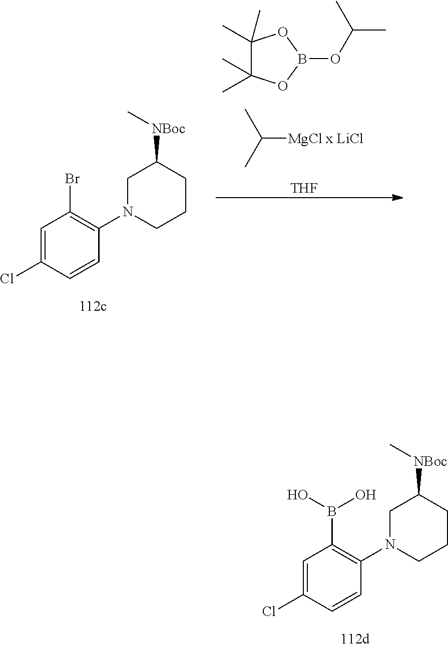

To the cooled to −30° C. solution of 112c (67 mg; 0.167 mmol) in dry THF (2 mL) a isopropyl magnesium chloride lithium chloride complex (1.3 M in THF; 0.38 mL; 0.498 mmol) was added dropwise and stirred in the temperature between −30° C. and 0° C. for 1 hour. Then another portion of isopropyl magnesium chloride lithium chloride complex (1.3 M in THF; 0.09 mL; 0.111 mmol) was added dropwise and stirred at the temperature between −30° C. and 0° C. for 5 hours. Then the mixture was recooled to −30° C. and to this mixture 2-isopropoxy-4,4,5,5-tetramethyl-1,3,2-dioxaborolane (70 μL; 0.332 mmol) was added and then the reaction mixture was allowed to warm to room temperature and stirred overnight. The reaction progress was monitored by LC-MS. When analysis indicated completion of the reaction, to the reaction mixture a saturated aqueous solution of NH₄Cl (1 mL) was added, diluted with water and the product was extracted with AcOEt. The organic layer was dried over anhydrous Na₂SO₄, filtered and concentrated in vacuo. The crude product was used to the next step without any additional purification.

ESI-MS m/z for C₁₇H₂₇BClN₂O₄ found 368.9/371.0 [M+H]⁺; Rₜ=1.54 min (Method A)

### Step 5.

### Synthesis of tert-butyl ((3S)-1-(4-chloro-2-(6-((6,6-dimethyl-2,4-dioxo-3-azabicyclo[3.1.0]hexan-3-yl)methyl)pyrrolo[2,1-f][1,2,4]triazin-4-yl)phenyl)piperidin-3-yl)(methyl)carbamate (112e)

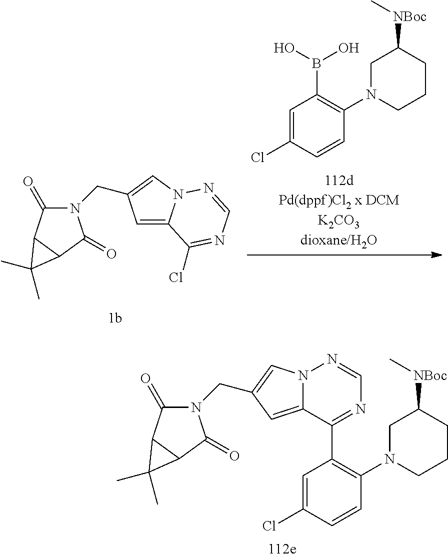 [SMILES: CN(C(=O)OC(C)(C)C)[C@H]1CCCN(c2ccc(Cl)cc2B(O)O)C1 ; CC1(C)C2C(=O)N(Cc3cc4c(Cl)ncnn4c3)C(=O)C21 ; CN(C(=O)OC(C)(C)C)[C@H]1CCCN(c2ccc(Cl)cc2-c2ncnn3cc(CN4C(=O)C5C(C4=O)C5(C)C)cc23)C1]

The title compound (112e) was obtained from 1b (30 mg; 0.098 mmol) and from 112d (the crude product) according to the General Procedure IV in 16% yield (per two steps)(16 mg; 0.027 mmol). The crude product was purified by flash column chromatography on silica (DCM/MeOH, 100:0 to 80:20, v/v, 20 min, 13 mL/min).

ESI-MS m/z for C₃₁H₃₈ClN₆O₄ found 593.5/595.1 [M+H]⁺; Rₜ=1.88 min (Method A)

### Step 6.

### Synthesis of 3-((4-(5-chloro-2-((S)-3-(methylamino)piperidin-1-yl)phenyl)pyrrolo[2,1-f][1,2,4]triazin-6-yl)methyl)-6,6-dimethyl-3-azabicyclo[3.1.0]hexane-2,4-dione hydrochloride (112)

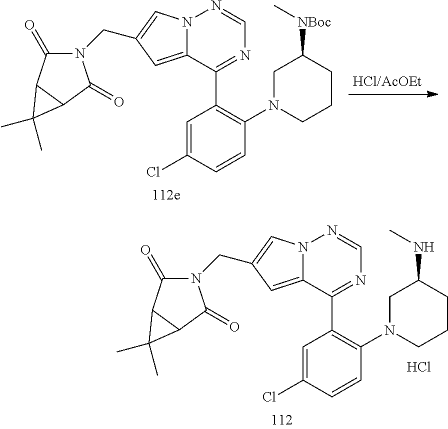 [SMILES: CN(C(=O)OC(C)(C)C)[C@H]1CCCN(c2ccc(Cl)cc2-c2ncnn3cc(CN4C(=O)C5C(C4=O)C5(C)C)cc23)C1 ; CN[C@H]1CCCN(c2ccc(Cl)cc2-c2ncnn3cc(CN4C(=O)C5C(C4=O)C5(C)C)cc23)C1 ; Cl]

The title compound (112) was obtained as a hydrochloride salt from 112e (16 mg; 0.027 mmol) according to the General Procedure IIIa in 89% yield (12.5 mg; 0.024 mmol). The crude product was purified by preparative reversed-phase column chromatography (column: Cosmosil Cholester 20×250 mm, water+0.3‰ TFA/MeCN, 99:1 to 40:60, 30 min, 20 mL/min, Rₜ=19.40 min).

ESI-MS m/z for C₂₆H₃₀ClN₆O₂ found 493.4/495.4 [M+H]⁺; Rₜ=1.16 min (Method A); ¹H NMR (700 MHz, Methanol-d₄) δ 8.64-8.49 (m, 1H), 8.12-8.08 (m, 1H), 7.65-7.52 (m, 2H), 7.37-7.29 (m, 1H), 6.93-6.78 (m, 1H), 4.76-4.64 (m, 2H), 3.56-3.46 (m, 1H), 3.17-2.98 (m, 2H), 2.96-2.82 (m, 2H), 2.70 (s, 3H), 2.66-2.58 (m, 1H), 2.52-2.42 (m, 2H), 2.05-1.96 (m, 1H), 1.57-1.35 (m, 2H), 1.28-1.19 (m, 3H), 1.10-1.00 (m, 3H).

### Example 113

### Synthesis of 3-((4-(5-chloro-3-methyl-2-(((5S)-5-methylmorpholin-2-yl)methyl)phenyl)pyrrolo[2,1-f][1,2,4]triazin-6-yl)methyl)-1-isopropylpyrimidine-2,4(1H,3H)-dione hydrochloride (113)

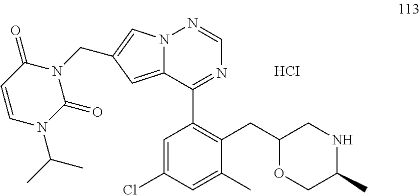

The title compound (113) was obtained as a hydrochloride salt as a single isomer in 1% overall yield in a similar way to Example 110 with the exception that, in the ninth step of the synthesis, 2-iodopropane (stabilized with copper) was used instead of methyl iodide, and in the last step of the synthesis, the crude product was purified by preparative reversed-phase column chromatography (C-18, water+0.3‰ HCl (36%)/MeCN, 98:2 to 30:70, 30 min, 20 mL/min, Rₜ=17.20 min).

ESI-MS m/z for C₂₇H₃₂ClN₆O₃ found 523.4/525.4 [M+H]⁺; Rₜ=1.16 min (Method A); ¹H NMR (700 MHz, Methanol-d₄) δ 8.66 (s, 1H), 8.45 (d, J=1.4 Hz, 1H), 7.69 (d, J=8.0 Hz, 1H), 7.59 (d, J=2.3 Hz, 1H), 7.46 (d, J=2.3 Hz, 1H), 7.13-7.08 (m, 1H), 5.79 (d, J=8.0 Hz, 1H), 5.28-5.22 (m, 2H), 4.81-4.77 (m, 1H), 3.74-3.67 (m, 2H), 3.30-3.25 (m, 1H), 3.22-3.16 (m, 1H), 3.15-3.09 (m, 1H), 3.04-2.98 (m, 1H), 2.87-2.78 (m, 2H), 2.50 (s, 3H), 1.39-1.25 (m, 6H), 1.14 (d, J=6.5 Hz, 3H).

### Example 114

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1-ethylpyrimidine-2,4(1H,3H)-dione hydrochloride (114)

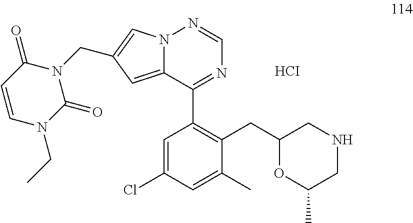

The title compound (114) was obtained as a hydrochloride salt as a single isomer in 2% overall yield in a similar way to Example 110 with the exception that, in the first step of the synthesis, (S)-1-aminopropan-2-ol was used instead of (S)-2-aminopropan-1-ol, in the ninth step of the synthesis, iodoethane was used instead of methyl iodide, and in the last step of the synthesis, the crude product was purified by preparative reversed-phase column chromatography (C-18, water+0.3‰ HCl (36%)/MeCN, 99:1 to 20:80, 30 min, 20 mL/min, Rₜ=15.63 min).

ESI-MS m/z for C₂₆H₃₀ClN₆O₃ found 509.5/511.5 [M+H]⁺; Rₜ=1.04 min (Method A); ¹H NMR (700 MHz, Methanol-d₄) δ 8.51 (s, 1H), 8.12 (d, J=1.4 Hz, 1H), 7.59 (d, J=7.8 Hz, 1H), 7.49 (d, J=1.9 Hz, 1H), 7.42 (d, J=2.1 Hz, 1H), 6.77 (d, J=1.3 Hz, 1H), 5.74 (d, J=7.8 Hz, 1H), 5.23-5.17 (m, 2H), 3.84-3.80 (m, 2H), 3.79-3.76 (m, 1H), 3.51-3.47 (m, 1H), 3.19-3.14 (m, 2H), 3.07-3.04 (m, 1H), 2.94-2.91 (m, 1H), 2.67-2.63 (m, 1H), 2.53-2.49 (m, 1H), 2.48 (s, 3H), 1.26 (t, J=7.2 Hz, 3H), 0.74 (d, J=6.2 Hz, 3H).

### Example 115

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1,5-dimethylpyrimidine-2,4(1H,3H)-dione hydrochloride (115)

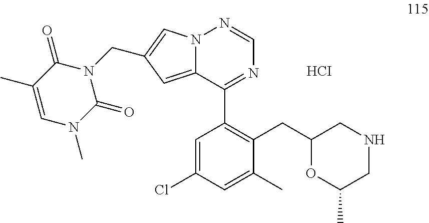

The title compound (115) was obtained as a hydrochloride salt as a single isomer in 0.6% overall yield in a similar way to Example 110 with the exception that, in the first step of the synthesis, (S)-1-aminopropan-2-ol was used instead of (S)-2-aminopropan-1-ol, in the sixth step of the synthesis, the compound 115a′ (the synthesis of this compound was described below) was used instead of the compound 89b, and in the last step of the synthesis, the crude product was purified by preparative reversed-phase column chromatography (C-18, water+0.3‰ HCl (36%)/MeCN, 99:1 to 20:80, 30 min, 20 mL/min, Rₜ=15.57 min).

ESI-MS m/z for C₂₆H₃₀ClN₆O₃ found 509.4/511.4 [M+H]⁺; Rₜ=1.05 min (Method A); ¹H NMR (700 MHz, Methanol-d₄) δ 8.63 (s, 1H), 8.38-8.35 (m, 1H), 7.58 (d, J=2.1 Hz, 1H), 7.48 (d, J=2.2 Hz, 1H), 7.43 (d, J=1.1 Hz, 1H), 7.06 (s, 1H), 5.28-5.21 (m, 2H), 3.80-3.77 (m, 1H), 3.55-3.51 (m, 1H), 3.35 (s, 3H), 3.23-3.21 (m, 1H), 3.09-3.04 (m, 2H), 2.99-2.96 (m, 1H), 2.69-2.65 (m, 1H), 2.53-2.50 (m, 1H), 2.49 (s, 3H), 1.89 (s, 3H), 0.75 (d, J=6.2 Hz, 3H).

### Synthesis of tert-butyl 3-((4-chloropyrrolo[2,1-f][1,2,4]triazin-6-yl)methyl)-5-methyl-2,4-dioxo-3,4-dihydropyrimidine-1(2H)-carboxylate (115a′)

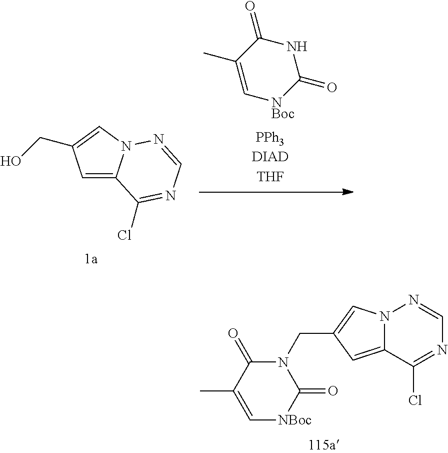 [SMILES: Cc1cn(C(=O)OC(C)(C)C)c(=O)[nH]c1=O ; OCc1cc2c(Cl)ncnn2c1 ; Cc1cn(C(=O)OC(C)(C)C)c(=O)n(Cc2cc3c(Cl)ncnn3c2)c1=O]

To a cooled to −10° C. solution of 1a (300 mg; 1.63 mmol), tert-butyl 5-methyl-2,4-dioxo-3,4-dihydropyrimidine-1(2H)-carboxylate (369 mg; 1.63 mmol) and PPh₃ (513 mg; 1.96 mmol) in dry THF (6.1 mL) DIAD (0.42 mL; 2.12 mmol) was added dropwise. The resulting mixture was stirred at room temperature for 2 hours. Then the reaction mixture was concentrated in vacuo and the residue was purified by flash column chromatography on silica (hexane/AcOEt, 100:0 to 0:100, v/v, 40 min, 13 mL/min). Compound 115a′ was obtained in 58% yield (369 mg; 0.94 mmol).

ESI-MS m/z for C₁₇H₁₉ClN₅O₄ found 392.2/394.2 [M+H]⁺; Rₜ=1.53 min (Method A)

### Example 116

### Synthesis of 3-((4-(5-chloro-3-fluoro-2-(morpholin-2-ylmethyl)phenyl)pyrrolo[2,1-f][1,2,4]triazin-6-yl)methy)-1-methylpyrimidine-2,4(1H,3H)-dione hydrochloride (116)

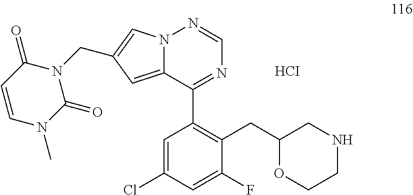

### Step 1.

### Synthesis of 2-(2-bromo-4-chloro-6-fluorobenzyl)morpholine (116a)

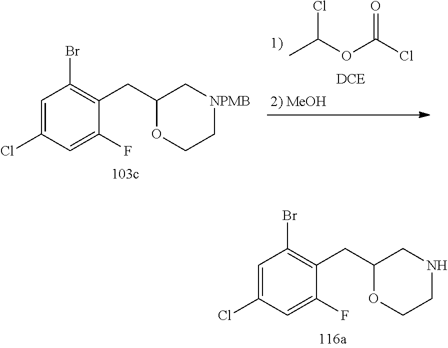

To the solution of 103c (394 mg; 0.919 mmol) in DCE (15 mL) 1-chloroethyl carbonochloridate (397 μL; 3.680 mmol) was added dropwise and this mixture was then stirred at 80° C. for 2 hours. The reaction progress was monitored by LC-MS. After this time, the solvent was evaporated in vacuo and the residue was dissolved in MeOH (9.7 mL) and whole was refluxed for 1 hour. Then the solvent was evaporated in vacuo and the crude product was used to the next step without any additional purification.

ESI-MS m/z for C₁₁H₁₃BrClFNO found 307.8/309.8 [M+H]⁺; Rₜ=1.12 min (Method C)

### Step 2.

### Synthesis of tert-butyl 2-(2-bromo-4-chloro-6-fluorobenzyl)morpholine-4-carboxylate (116b)

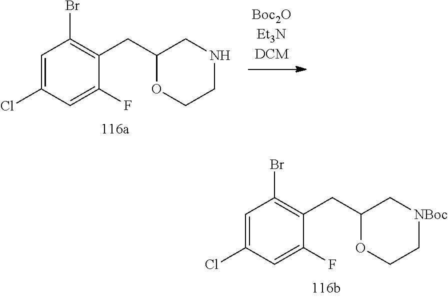 [SMILES: Fc1cc(Cl)cc(Br)c1CC1CNCCO1 ; CC(C)(C)OC(=O)N1CCOC(Cc2c(F)cc(Cl)cc2Br)C1]

To the solution of 116a (the crude product) in DCM (9 mL) Boc₂O (301 mg; 1.38 mmol) and Et₃N (0.19 mL; 1.38 mmol) were added and this mixture was then stirred at room temperature overnight. The reaction progress was monitored by LC-MS. When analysis indicated completion of the reaction, the reaction mixture was diluted with DCM and quenched with water. An aqueous phase (pH=8) was extracted with DCM. The combined organic phases were dried over anhydrous Na₂SO₄, filtered and concentrated in vacuo and the residue was purified by flash column chromatography on silica (hexane/AcOEt, 100:0 to 0:100, v/v, 20 min, 28 mL/min). The crude product was then used to the next step without further purification.

ESI-MS m/z for C₁₁H₁₃BrClFNO found 307.9/309.9 [M+H-Boc]⁺; Rₜ=1.89 min (Method C)

### Step 3.

### Synthesis of tert-butyl 2-(4-chloro-2-fluoro-6-(4,4,5,5-tetramethyl-1,3,2-dioxaborolan-2-yl)benzyl)morpholine-4-carboxylate (116c)

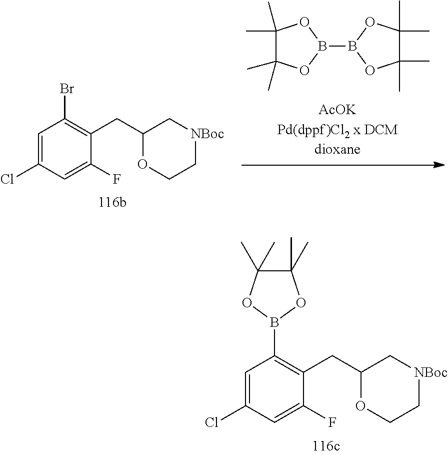

In a Schlenk flask was placed 116b (the crude product), bis(pinacolato)diboron (472 mg; 1.840 mmol), AcOK (274 mg; 2.760 mmol) and Pd(dppf)Cl₂×DCM (38 mg; 0.046 mmol) and dioxane (4.6 mL). The reaction mixture was intensively flushed with Ar. The mixture was capped and stirred at 100° C. overnight. The reaction progress was monitored by LC-MS. When analysis indicated completion of the reaction, the reaction mixture was concentrated in vacuo. The residue was then purified by flash column chromatography on silica (hexane/AcOEt, 100:0 to 30:70, v/v, 15 min, 15 mL/min). Compound 116c was obtained in 99% yield (414 mg; 0.910 mmol).

ESI-MS m/z for C₁₇H₂₅BClFNO₃ found 355.9/357.9 [M+H-Boc]⁺; Rₜ=0.98 min (Method C)

### Step 4.

### Synthesis of tert-butyl 2-(4-chloro-2-(6-((2,6-dioxo-3,6-dihydropyrimidin-1(2H)-yl)methyl)pyrrolo[2,1-f][1,2,4]triazin-4-yl)-6-fluorobenzyl)morpholine-4-carboxylate (116d)

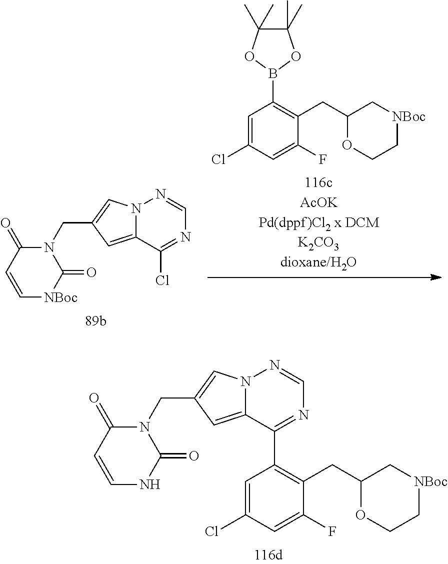 [SMILES: CC(C)(C)OC(=O)N1CCOC(Cc2c(F)cc(Cl)cc2B2OC(C)(C)C(C)(C)O2)C1 ; CC(C)(C)OC(=O)n1ccc(=O)n(Cc2cc3c(Cl)ncnn3c2)c1=O ; CC(C)(C)OC(=O)N1CCOC(Cc2c(F)cc(Cl)cc2-c2ncnn3cc(Cn4c(=O)cc[nH]c4=O)cc23)C1]

The title compound (116d) was obtained from 89b (332 mg; 0.878 mmol) and from 116c (400 mg; 0.878 mmol) according to the General Procedure IV in 99% yield (495 mg; 0.869 mmol). The crude product was purified by flash column chromatography on silica (DCM/MeOH, 100:0 to 90:10, v/v, 20 min, 15 mL/min).

ESI-MS m/z for C₂₇H₂₉ClFN₆O₅ found 571.3/573.3 [M+H]⁺; Rₜ=1.47 min (Method C)

### Step 5.

### Synthesis of tert-butyl 2-(4-chloro-2-fluoro-6-(6-((3-methyl-2,6-dioxo-3,6-dihydropyrimidin-1(2H)-yl)methyl)pyrrolo[2,1-f][1,2,4]triazin-4-yl)benzyl)morpholine-4-carboxylate (116e)

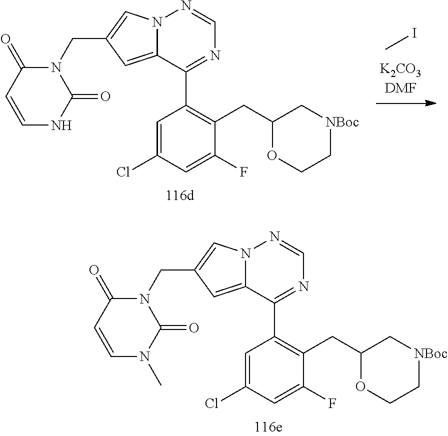

To the solution of 116d (200 mg; 0.350 mmol) in dry DMF (5 mL) K₂CO₃ (99 mg; 0.701 mmol) and methyl iodide (22 μL; 0.350 mmol) were added. The resulting mixture was stirred at room temperature overnight. The reaction progress was monitored by LC-MS. When analysis indicated completion of the reaction, the reaction mixture was diluted with AcOEt and quenched with water (pH=9). An aqueous phase was extracted with AcOEt. The combined organic phases were then dried over anhydrous Na₂SO₄, filtered and concentrated in vacuo and the residue was purified by flash column chromatography on silica (hexane/AcOEt, 100:0 to 0:100, v/v, 20 min, 20 mL/min). Compound 116e was obtained in 50% yield (102 mg; 0.175 mmol).

ESI-MS m/z for C₂₈H₃₁ClFN₆O₅ found 585.2/587.2 [M+H]; Rₜ=1.57 min (Method C)

### Step 6.

### Synthesis of 3-((4-(5-chloro-3-fluoro-2-(morpholin-2-ylmethyl)phenyl)pyrrolo[2,1-f][1,2,4]triazin-6-yl)methyl)-1-methylpyrimidine-2,4(1H,3H)-dione hydrochloride (116)

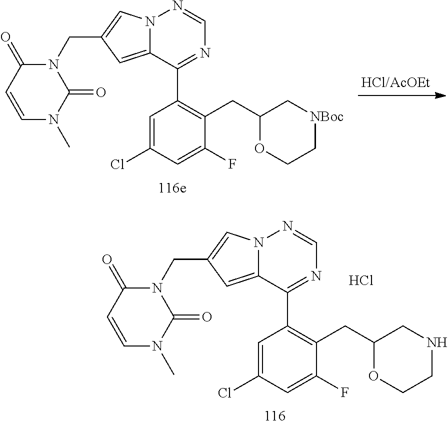 [SMILES: Cn1ccc(=O)n(Cc2cc3c(-c4cc(Cl)cc(F)c4CC4CN(C(=O)OC(C)(C)C)CCO4)ncnn3c2)c1=O ; Cn1ccc(=O)n(Cc2cc3c(-c4cc(Cl)cc(F)c4CC4CNCCO4)ncnn3c2)c1=O ; Cl]

The title compound (116) was obtained as a hydrochloride salt from 116e (102 mg; 0.175 mmol) according to the General Procedure IIIa in 29% yield (26 mg; 0.050 mmol). The crude product was purified by preparative reversed-phase column chromatography (C-18, water+1‰ HCl (36%)/MeCN, 95:5 to 5:95, 30 min, 20 mL/min, Rₜ=13.66 min).

ESI-MS m/z for C₂₃H₂₃ClFN₆O₃ found 485.3/487.0 [M+H]⁺; Rₜ=0.91 min (Method C); ¹H NMR (400 MHz, Methanol-d₄) δ 8.67 (s, 1H), 8.42 (d, J=1.3 Hz, 1H), 7.63-7.44 (m, 3H), 7.11 (d, J=1.3 Hz, 1H), 5.72 (d, J=7.8 Hz, 1H), 5.23 (s, 2H), 3.89-3.78 (m, 1H), 3.71-3.61 (m, 1H), 3.58-3.43 (m, 1H), 3.36 (s, 3H), 3.29-3.22 (m, 1H), 3.13-3.00 (m, 2H), 2.97-2.81 (m, 2H), 2.80-2.69 (m, 1H); ¹⁹F NMR (376 MHz, Methanol-d₄) δ −111.75 (d, J=9.5 Hz).

### Example 117

### Synthesis of 1-((4-(5-chloro-3-methyl-2-(((6S)-6-methylmorpholin-2-yl)methyl)phenyl)pyrrolo[2,1-f][1,2,4]triazin-6-yl)methyl)-4-methylpiperazine-2,6-dione hydrochloride (117)

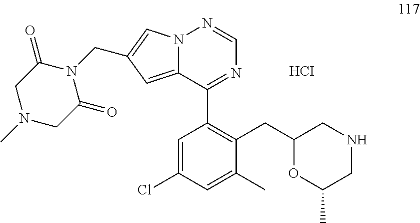

The title compound (117) was obtained as a hydrochloride salt as a single isomer in 0.6% overall yield in a similar way to Example 110 with the exception that, in the first step of the synthesis, (S)-1-aminopropan-2-ol was used instead of (S)-2-aminopropan-1-ol, in the sixth step of the synthesis, the compound 117a′ (the synthesis of this compound was described below) was used instead of the compound 89b, and in the last step of the synthesis, the reaction was carried out without addition of MeOH and the crude product was purified by preparative reversed-phase column chromatography (C-18, water+1‰ HCl (36%)/MeCN, 95:5 to 5:95, 30 min, 20 mL/min, Rₜ=14.00 min) and then by preparative TLC (SiO₂; DCM:MeOH:HCl (36%); 90:10:0.1, 1× eluted).

ESI-MS m/z for C₂₅H₃₀ClN₆O₃ found 497.2/499.2 [M+H]⁺; Rₜ=1.09 min (Method C); ¹H NMR (400 MHz, Methanol-d₄) δ 8.65 (s, 1H), 8.38 (d, J=1.4 Hz, 1H), 7.58-7.54 (m, 1H), 7.46 (d, J=2.3 Hz, 1H), 7.08 (d, J=1.4 Hz, 1H), 5.15 (s, 2H), 4.38-4.31 (m, 4H), 3.82-3.73 (m, 1H), 3.66-3.56 (m, 1H), 3.23-2.93 (m, 7H), 2.73-2.66 (m, 1H), 2.59-2.51 (m, 1H), 2.50 (s, 3H), 0.91 (d, J=6.3 Hz, 3H).

### Synthesis of 1-((4-chloropyrrolo[2,1-f][1,2,4]triazin-6-yl)methyl)-4-methylpiperazine-2,6-dione (117a′)

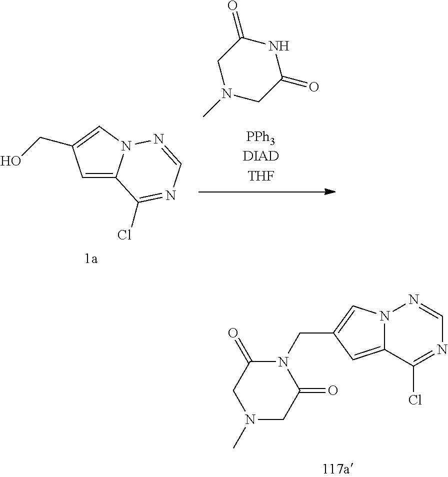

To a cooled to −10° C. solution of 1a (519 mg; 2.83 mmol), 4-methylpiperazine-2,6-dione (362 mg; 2.83 mmol) and PPh₃ (936 mg; 3.39 mmol) in dry THE (9.4 mL) DIAD (0.67 mL; 3.39 mmol) was added dropwise. The resulting mixture was stirred at room temperature for 1.5 hours. Then the reaction mixture was concentrated in vacuo and the residue was purified by flash column chromatography on silica (hexane/AcOEt, 100:0 to 40:60, v/v, 40 min, 30 mL/min). Compound 117a′ was obtained in 92% yield (with impurities of PPh₃₀)(767 mg; 2.62 mmol).

ESI-MS m/z for C₁₂H₁₃ClN₅O₂ found 294.0/296.0 [M+H]⁺; Rₜ=0.91 min (Method C)

### Example 118

### Synthesis of 3-((4-(3,5-dichloro-2-(morpholin-2-ylmethyl)phenyl)pyrrolo[2,1-f][1,2,4]triazin-6-yl)methyl)-1-methylpyrimidine-2,4(1H,3H)-dione hydrochloride (118)

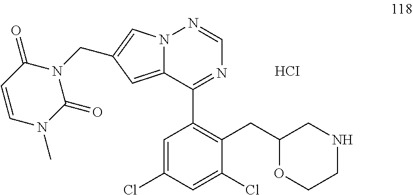

### Step 1.

### Synthesis of (2,4-dichloro-6-iodophenyl)methanol (118a)

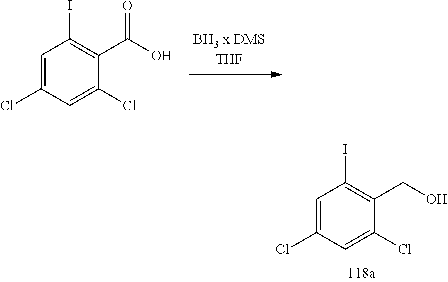 [SMILES: O=C(O)c1c(Cl)cc(Cl)cc1I ; OCc1c(Cl)cc(Cl)cc1I]

To the cooled to 0° C. solution of 2,4-dichloro-6-iodobenzoic acid (1.88 g; 5.93 mmol) in dry THE (20 mL) BH₃×DMS (1.13 mL; 11.90 mmol) was added dropwise and this mixture was then stirred at 80° C. for 4 hours. The reaction progress was monitored by LC-MS. When analysis indicated completion of the reaction, the reaction mixture was cooled to 0° C. and to this mixture MeOH was added and a whole was stirred at 80° C. for 20 minutes. Then the solvents were removed in vacuo and the residue was dissolved in AcOEt. Then to this solution water and an aqueous solution of NaHCO₃ were added (pH=8) and a whole was extracted with AcOEt (3×). The combined organic phases were dried over anhydrous Na₂SO₄, filtered, and concentrated in vacuo. The crude product was purified by flash column chromatography on silica (hexane/AcOEt, 100:0 to 30:70, v/v, 30 min, 30 mL/min). Compound 118a was obtained in 52% yield (924 mg; 3.06 mmol).

¹H NMR (400 MHz, DMSO-d₆) δ 7.93 (d, J=2.1 Hz, 1H), 7.66 (d, J=2.1 Hz, 1H), 5.19 (t, J=5.2 Hz, 1H), 4.67 (d, J=5.2 Hz, 2H).

### Step 2.

### Synthesis of 2-(bromomethyl)-1,5-dichloro-3-iodobenzene (118b)

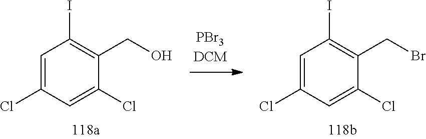 [SMILES: OCc1c(Cl)cc(Cl)cc1I ; Clc1cc(Cl)c(CBr)c(I)c1]

To a cooled to 0° C. solution of 118a (924 mg; 3.06 mmol) in dry DCM (12.7 mL) PBr₃ (583 μL; 6.22 mmol) was added dropwise. The resulting mixture was allowed to warm to room temperature and stirred for 3 hours. The reaction progress was monitored by LC-MS. When analysis indicated completion of the reaction, the reaction mixture was cooled to 0° C., diluted with DCM and quenched with a saturated aqueous solution of NaHCO₃. The layers were separated and an aqueous one was then extracted with DCM. The combined organic layers were dried over anhydrous Na₂SO₄, filtered and concentrated in vacuo. Compound 118b was obtained in 39% yield (441 mg; 1.21 mmol).

¹H NMR (400 MHz, DMSO-d₆) δ 8.00 (d, J=2.1 Hz, 1H), 7.75 (d, J=2.1 Hz, 1H), 4.77 (s, 2H).

### Step 3.

### Synthesis of 2-(2,4-dichloro-6-iodobenzyl)-4-(4-methoxybenzyl)morpholin-3-one (118c)

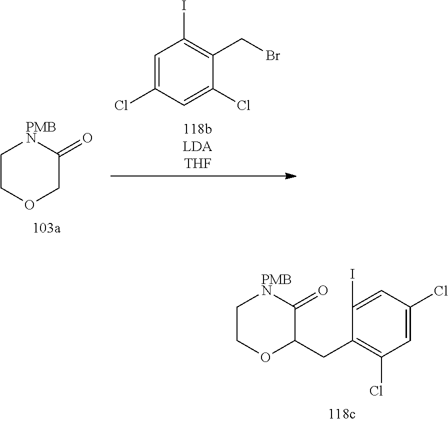

To the cooled to −78° C. solution of 103a (153 mg; 0.691 mmol) in THF (1 mL) LDA (1 M solution in THF/hexanes; 0.83 mL; 0.830 mmol) was added under an Argon atmosphere and a whole was then stirred for 10 minutes at this temperature. Then to this mixture a solution of 118b (253 mg; 0.692 mmol) in THE (1.3 mL) was added dropwise and the reaction mixture was allowed to warm to room temperature and stirred for 1 hour. The reaction progress was monitored by LC-MS. When analysis indicated completion of the reaction, the reaction mixture was diluted with AcOEt and quenched with a saturated aqueous solution of NH₄Cl. An aqueous phase was extracted with AcOEt. The combined organic phases were dried over anhydrous Na₂SO₄, filtered, and concentrated in vacuo. The crude product was purified by flash column chromatography on silica (hexane/AcOEt, 100:0 to 0:100, v/v, 30 min, 30 mL/min). Compound 118c was obtained in 43% yield (150 mg; 0.297 mmol).

ESI-MS m/z for C₁₉H₁₉Cl₂INO₃ found 505.9/507.8 [M+H]⁺; Rₜ=1.82 min (Method C); ¹H NMR (400 MHz, DMSO-d₆) δ 7.92 (d, J=2.1 Hz, 1H), 7.64 (d, J=2.1 Hz, 1H), 7.24-7.14 (m, 2H), 6.96-6.90 (m, 2H), 4.43-4.38 (m, 3H), 3.94-3.84 (m, 1H), 3.72 (s, 3H), 3.68-3.52 (m, 2H), 3.39-3.31 (m, 2H), 3.19-3.11 (m, 1H).

### Step 4.

### Synthesis of 2-(2,4-dichloro-6-iodobenzyl)-4-(4-methoxybenzyl)morpholine (118d)

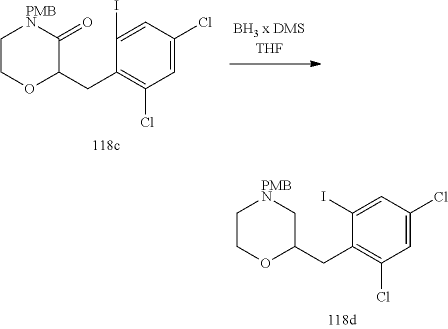

To the cooled to 0° C. solution of 118c (150 mg; 0.297 mmol) in dry THF (1 mL) BH₃×DMS (0.056 mL; 0.593 mmol) was added dropwise and this mixture was then stirred at 80° C. for 4 hours. The reaction progress was monitored by LC-MS. When analysis indicated completion of the reaction, the reaction mixture was cooled to 0° C. and to this mixture MeOH was added and a whole was stirred at 80° C. for 20 minutes. Then the solvents were removed in vacuo and the residue was dissolved in AcOEt. Then to this solution water and an aqueous solution of NaHCO₃ were added (pH=8) and a whole was extracted with AcOEt (3×). The combined organic phases were dried over anhydrous Na₂SO₄, filtered, and concentrated in vacuo. The crude product was purified by flash column chromatography on silica (hexane/AcOEt, 100:0 to 0:100, v/v, 30 min, 15 mL/min). Compound 118d was obtained in 67% yield (98 mg; 0.199 mmol).

ESI-MS m/z for C₁₉H₂₁Cl₂INO₂ found 492.0/493.8 [M+H]⁺; Rₜ=2.00 min (Method C); ¹H NMR (400 MHz, CDCl₃) δ 7.76 (d, J=2.1 Hz, 1H), 7.38 (d, J=2.1 Hz, 1H), 7.24-7.19 (m, 2H), 6.87-6.82 (m, 2H), 3.90-3.83 (m, 1H), 3.82-3.78 (m, 4H), 3.62-3.48 (m, 2H), 3.40-3.36 (m, 1H), 3.24-3.16 (m, 1H), 3.10-3.01 (m, 1H), 2.78-2.68 (m, 1H), 2.65-2.56 (m, 1H), 2.20-2.09 (m, 2H).

### Step 5.

### Synthesis of 2-(2,4-dichloro-6-(4,4,5,5-tetramethyl-1,3,2-dioxaborolan-2-yl)benzyl)-4-(4-methoxybenzyl)morpholine (118e)

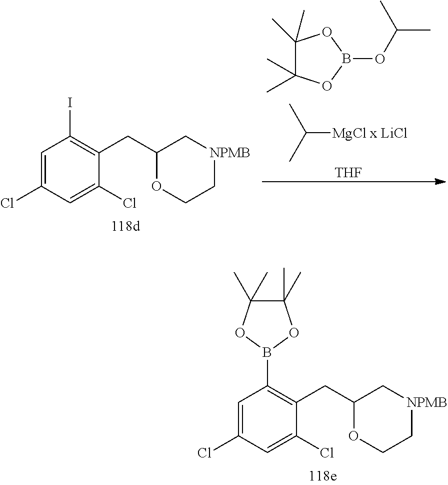

To the cooled to 0° C. solution of isopropyl magnesium chloride lithium chloride complex (1.3 M in THF; 0.70 mL; 0.914 mmol) in THE (1 mL) a solution of 118d (90 mg; 0.183 mmol) in dry THF (1 mL) was added dropwise and stirred at 0° C. for 30 minutes. Then to this mixture 2-isopropoxy-4,4,5,5-tetramethyl-1,3,2-dioxaborolane (112 μL; 0.549 mmol) was added and then the reaction mixture was allowed to warm to room temperature and stirred for 30 minutes. The reaction progress was monitored by LC-MS. When analysis indicated completion of the reaction, the reaction mixture was diluted with AcOEt and quenched with water. An aqueous phase was extracted with AcOEt. The combined organic phases were dried over anhydrous Na₂SO₄, filtered and concentrated in vacuo. The crude product was used to the next step without any additional purification.

ESI-MS m/z for C₂₅H₃₃BC12NO₄ found 492.1/494.1 [M+H]⁺; Rₜ=1.79 min (Method C)

### Step 6.

### Synthesis of tert-butyl 3-((4-(3,5-dichloro-2-((4-(4-methoxybenzyl)morpholin-2-yl)methyl)phenyl)pyrrolo[2,1-f][1,2,4]triazin-6-yl)methyl)-2,4-dioxo-3,4-dihydropyrimidine-1(2H)-carboxylate (118f)

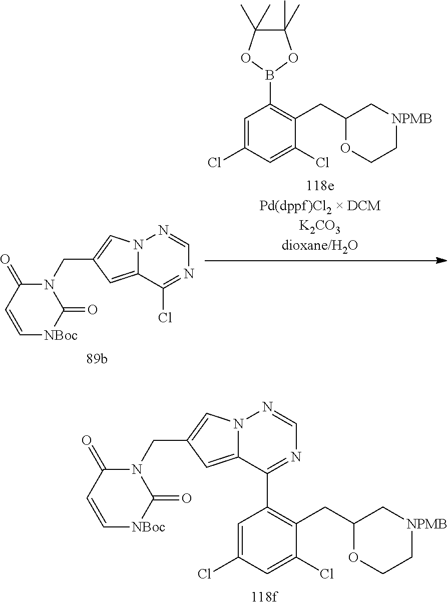

The title compound (118f) was obtained from 89b (69 mg; 0.183 mmol) and from 118e (the crude product) according to the General Procedure IV in 52% yield (per two steps)(67 mg; 0.095 mmol). The crude product was purified by flash column chromatography on silica (hexane/AcOEt, 100:0 to 0:100, v/v, 50 min, 20 mL/min).

ESI-MS m/z for C₃₀H₂₉Cl₂N₆O₄ found 607.3/609.1 [M+H-Boc]⁺; Rₜ=1.20 min (Method C); ¹H NMR (400 MHz, Methanol-d₄) δ 8.45 (s, 1H), 8.07 (d, J=1.5 Hz, 1H), 7.68 (d, J=2.2 Hz, 1H), 7.48 (d, J=2.2 Hz, 1H), 7.22 (d, J=7.6 Hz, 1H), 6.99-6.95 (m, 2H), 6.84-6.78 (m, 2H), 6.69 (d, J=1.5 Hz, 1H), 5.60 (d, J=7.6 Hz, 1H), 5.16 (s, 2H), 3.77 (s, 3H), 3.57-3.47 (m, 1H), 3.29-3.23 (m, 2H), 3.23-3.14 (m, 2H), 3.11-3.04 (m, 2H), 2.54-2.27 (m, 2H), 1.82-1.69 (m, 1H), 1.55-1.45 (m, 1H), 1.19 (s, 9H).

### Step 7.

### Synthesis of tert-butyl 3-((4-(3,5-dichloro-2-(morpholin-2-ylmethyl)phenyl)pyrrolo[2,1-f][1,2,4]triazin-6-yl)methyl)-2,4-dioxo-3,4-dihydropyrimidine-1(2H)-carboxylate (118g)

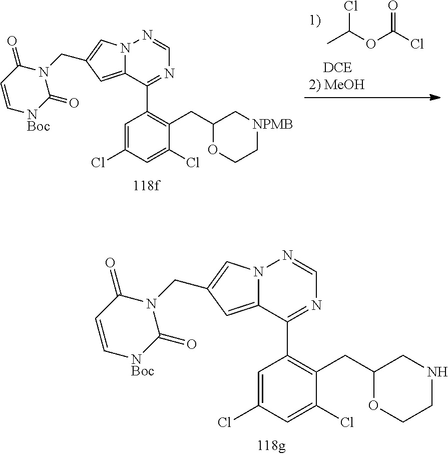

To the solution of 118f (67 mg; 0.095 mmol) in DCE (1 mL) 1-chloroethyl carbonochloridate (48 μL; 0.441 mmol) was added dropwise and this mixture was then stirred at 80° C. for 2 hours. The reaction progress was monitored by LC-MS. After this time, the solvents were evaporated in vacuo and the residue was dissolved in MeOH (3 mL) and a whole was then stirred at 75° C. for 1 hour. Then the solvent was evaporated in vacuo and the crude product was used to the next step without any additional purification.

ESI-MS m/z for C₂₂H₂₁Cl₂N₆O₃ found 487.0/488.9 [M+H-Boc]⁻; Rₜ=0.94 min (Method C)

### Step 8.

### Synthesis of tert-butyl 2-(2-(6-((3-(tert-butoxycarbonyl)-2,6-dioxo-3,6-dihydropyrimidin-1(2H)-yl)methyl)pyrrolo[2,1-f][1,2,4]triazin-4-yl)-4,6-dichlorobenzyl)morpholine-4-carboxylate (118h)

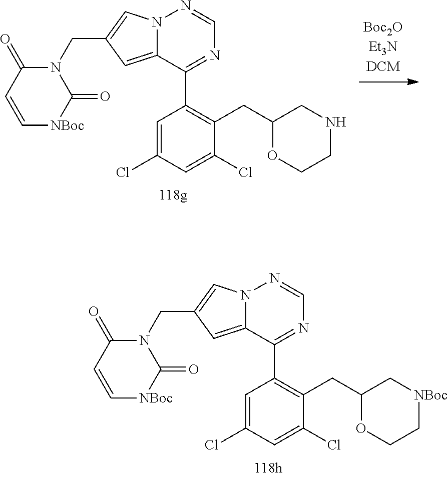 [SMILES: CC(C)(C)OC(=O)n1ccc(=O)n(Cc2cc3c(-c4cc(Cl)cc(Cl)c4CC4CNCCO4)ncnn3c2)c1=O ; CC(C)(C)OC(=O)N1CCOC(Cc2c(Cl)cc(Cl)cc2-c2ncnn3cc(Cn4c(=O)ccn(C(=O)OC(C)(C)C)c4=O)cc23)C1]

To the solution of 118g (the crude product) in DCM (1.2 mL) Boc₂O (30 mg; 0.138 mmol) and Et₃N (0.02 mL; 0.138 mmol) were added and this mixture was then stirred at room temperature overnight. The reaction progress was monitored by LC-MS. When analysis indicated completion of the reaction, the reaction mixture was diluted with DCM and quenched with water. An aqueous phase (pH=8) was extracted with DCM. The combined organic phases were dried over anhydrous Na₂SO₄, filtered and concentrated in vacuo. Compound 118h was obtained in 97% yield (per two steps)(63 mg; 0.092 mmol).

ESI-MS m/z for C₃₂H₃₇Cl₂N₆O₇ found 687.2/689.2 [M+H]⁺; Rₜ=1.96 min (Method C)

### Step 9.

### Synthesis of tert-butyl 2-(2,4-dichloro-6-(6-((2,6-dioxo-3,6-dihydropyrimidin-1(2H)-yl)methyl)pyrrolo[2,1-f][1,2,4]triazin-4-yl)benzyl)morpholine-4-carboxylate (118i)

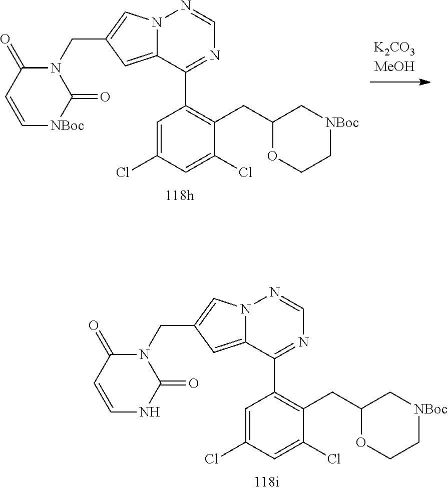 [SMILES: CC(C)(C)OC(=O)N1CCOC(Cc2c(Cl)cc(Cl)cc2-c2ncnn3cc(Cn4c(=O)ccn(C(=O)OC(C)(C)C)c4=O)cc23)C1 ; CC(C)(C)OC(=O)N1CCOC(Cc2c(Cl)cc(Cl)cc2-c2ncnn3cc(Cn4c(=O)cc[nH]c4=O)cc23)C1]

To the solution of 118h (70 mg; 0.102 mmol) in MeOH (1.5 mL) K₂CO₃ (56 mg; 0.407 mmol) wad added at room temperature and this mixture was then stirred at 45° C. for 1 hour. The reaction progress was monitored by LC-MS. When analysis indicated completion of the reaction, the reaction mixture was diluted with AcOEt and quenched with brine with citric acid. An aqueous phase (pH=6/7) was extracted with AcOEt. The combined organic phases were dried over anhydrous Na₂SO₄, filtered and concentrated in vacuo. Compound 118i was obtained in 97% yield (58 mg; 0.099 mmol).

ESI-MS m/z for C₂₇H₂₉Cl₂N₆O₅ found 587.2/589.2 [M+H]; Rₜ=1.55 min (Method C)

### Step 10.

### Synthesis of tert-butyl 2-(2,4-dichloro-6-(6-((3-methyl-2,6-dioxo-3,6-dihydropyrimidin-1(2H)-yl)methyl)pyrrolo[2,1-f][1,2,4]triazin-4-yl)benzyl)morpholine-4-carboxylate (118j)

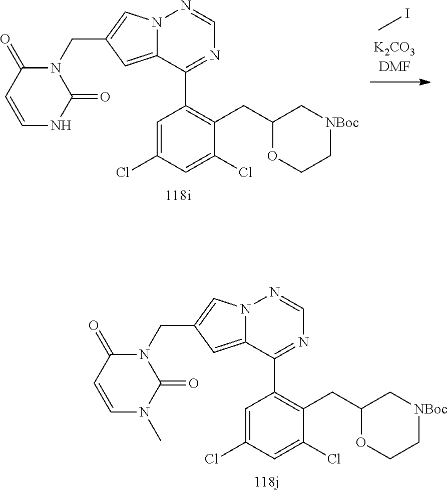

To the solution of 118i (58 mg; 0.099 mmol) in dry DMF (2 mL) K₂CO₃ (28 mg; 0.197 mmol) and methyl iodide (6.2 μL; 0.099 mmol) were added. The resulting mixture was stirred at room temperature overnight. Then another portion of methyl iodide (3.1 μL; 0.049 mmol) was added and the resulting mixture was stirred at room temperature for 7 hours. Then to this mixture next portions of methyl iodide (6.2 μL; 0.099 mmol) and K₂CO₃ (14 mg; 0.099 mmol) were added and stirred at room temperature. The reaction progress was monitored by LC-MS. When analysis indicated completion of the reaction, the reaction mixture was diluted with AcOEt and quenched with water (pH=9). An aqueous phase was extracted with AcOEt, then neutralized with 1 M HCl to pH=7 and extracted with AcOEt. The combined organic phases were dried over anhydrous Na₂SO₄, filtered and concentrated in vacuo and the residue was purified by flash column chromatography on silica (hexane/AcOEt, 100:0 to 0:100, v/v, 30 min, 20 mL/min) and then by TLC plate (DCM/MeOH, 98:2, v/v). Compound 118j was obtained in 11% yield (6.7 mg; 0.011 mmol).

ESI-MS m/z for C₂₈H₃₁Cl₂N₆O₅ found 601.2/603.2 [M+H]⁺; Rₜ=1.65 min (Method C); ¹H NMR (400 MHz, Methanol-d₄) δ 8.45 (s, 1H), 8.03 (d, J=1.5 Hz, 1H), 7.70 (d, J=2.2 Hz, 1H), 7.53 (d, J=7.8 Hz, 1H), 7.47 (d, J=2.2 Hz, 1H), 6.65 (d, J=1.5 Hz, 1H), 5.71 (d, J=7.8 Hz, 1H), 5.19 (s, 2H), 3.79-3.66 (m, 1H), 3.61-3.52 (m, 1H), 3.37-3.33 (m, 4H), 3.28-3.23 (m, 1H), 3.13-2.97 (m, 3H), 2.64-2.30 (m, 2H), 1.39 (s, 9H).

### Step 11.

### Synthesis of 3-((4-(3,5-dichloro-2-(morpholin-2-ylmethyl)phenyl)pyrrolo[2,1-f][1,2,4]triazin-6-yl)methyl)-1-methylpyrimidine-2,4(1H,3H)-dione hydrochloride (118)

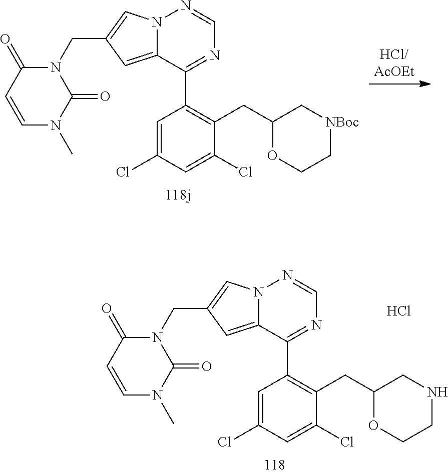 [SMILES: Cn1ccc(=O)n(Cc2cc3c(-c4cc(Cl)cc(Cl)c4CC4CN(C(=O)OC(C)(C)C)CCO4)ncnn3c2)c1=O ; Cn1ccc(=O)n(Cc2cc3c(-c4cc(Cl)cc(Cl)c4CC4CNCCO4)ncnn3c2)c1=O ; Cl]

The title compound (118) was obtained as a hydrochloride salt from 118j (6.7 mg; 0.011 mmol) according to the General Procedure IIIa in 54% yield (3 mg; 0.006 mmol). The crude product was purified by preparative reversed-phase column chromatography (C-18, water+1‰ HCl (36%)/MeCN, 95:5 to 5:95, 30 min, 20 mL/min, Rₜ=14.25 min).

ESI-MS m/z for C₂₃H₂₃Cl₂N₆O₃ found 501.1/503.1 [M+H]⁺; Rₜ=0.99 min (Method C); ¹H NMR (400 MHz, Methanol-d₄) δ 8.62 (s, 1H), 8.32 (d, J=1.4 Hz, 1H), 7.84 (d, J=2.2 Hz, 1H), 7.59 (d, J=2.2 Hz, 1H), 7.57 (d, J=7.8 Hz, 1H), 6.98-6.94 (m, 1H), 5.74 (d, J=7.8 Hz, 1H), 5.24 (s, 2H), 3.91-3.81 (m, 1H), 3.71-3.61 (m, 1H), 3.52-3.42 (m, 1H), 3.38 (s, 3H), 3.29-3.24 (m, 1H), 3.19-3.12 (m, 1H), 3.11-2.99 (m, 2H), 2.94-2.83 (m, 1H), 2.80-2.70 (m, 1H).

### Example 119

### Synthesis of 1-((4-(5-chloro-3-fluoro-2-(((6S)-6-methylmorpholin-2-yl)methyl)phenyl)pyrrolo[2,1-f][1,2,4]triazin-6-yl)methyl)-4-methylpiperazine-2,6-dione hydrochloride (119)

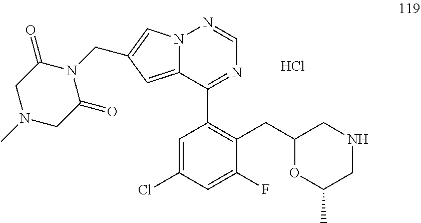 [SMILES: C[C@H]1CNCC(Cc2c(F)cc(Cl)cc2-c2ncnn3cc(CN4C(=O)CN(C)CC4=O)cc23)O1 ; Cl]

### Step 1.

### Synthesis of (6S)-2-(2-bromo-4-chloro-6-fluorobenzyl)-4-(4-methoxybenzyl)-6-methylmorpholin-3-one (119a)

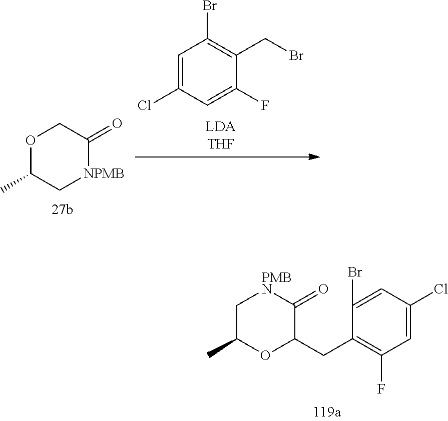

To the cooled to −78° C. solution of 27b (296 mg; 1.26 mmol) in dry THE (1.5 mL) LDA (1 M solution in THF/hexanes; 1.51 mL; 1.51 mmol) was added under a Nitrogen atmosphere and a whole was then stirred for 10 minutes at this temperature. Then to this mixture a solution of 1-bromo-2-(bromomethyl)-5-chloro-3-fluorobenzene (381 mg; 1.26 mmol) in THE (2.7 mL) was added dropwise and the reaction mixture was allowed to warm to room temperature and stirred for 1 hour. The reaction progress was monitored by LC-MS. When analysis indicated completion of the reaction, the reaction mixture was diluted with AcOEt and quenched with a saturated aqueous solution of NH₄Cl. An aqueous phase was extracted with AcOEt. The combined organic phases were dried over anhydrous Na₂SO₄, filtered, and concentrated in vacuo. The crude product was purified by flash column chromatography on silica (hexane/AcOEt, 100:0 to 0:100, v/v, 30 min, 25 mL/min). Compound 119a was obtained in 76% yield (438 mg; 0.96 mmol).

ESI-MS m/z for C₂₀H₂₁BrClFNO₃ found 456.0/458.0 [M+H]⁺; Rₜ=1.77 min (Method C); ¹H NMR (400 MHz, DMSO-d₆) δ 7.67-7.59 (m, 1H), 7.52-7.43 (m, 1H), 7.22-7.11 (m, 2H), 6.95-6.82 (m, 2H), 4.47-4.37 (m, 2H), 4.36-4.27 (m, 1H), 3.89-3.75 (m, 1H), 3.72 (s, 3H), 3.44-3.36 (m, 1H), 3.18-2.90 (m, 3H), 1.07-0.96 (m, 3H).

### Step 2.

### Synthesis of (6S)-2-(2-bromo-4-chloro-6-fluorobenzyl)-4-(4-methoxybenzyl)-6-methylmorpholine (119b)

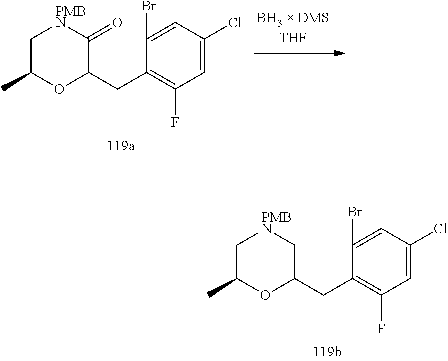

To the cooled to 0° C. solution of 119a (438 mg; 0.96 mmol) in dry THF (3.2 mL) BH₃×DMS (0.18 mL; 1.92 mmol) was added dropwise and this mixture was then stirred at 80° C. for 4 hours. The reaction progress was monitored by LC-MS. When analysis indicated completion of the reaction, the reaction mixture was cooled to 0° C. and to this mixture MeOH was added and a whole was stirred at 75° C. for 1 hour. Then the solvents were removed in vacuo and the residue was purified by flash column chromatography on silica (hexane/AcOEt, 100:0 to 30:70, v/v, 20 min, 30 mL/min). Compound 119b was obtained in 92% yield (390 mg; 0.88 mmol).

ESI-MS m/z for C₂₀H₂₂BrClFNO₂ found 442.0/443.9 [M+H]⁺; Rₜ=1.97 min (Method C)

### Step 3.

### Synthesis of (6S)-2-(2-bromo-4-chloro-6-fluorobenzyl)-6-methylmorpholine (119c)

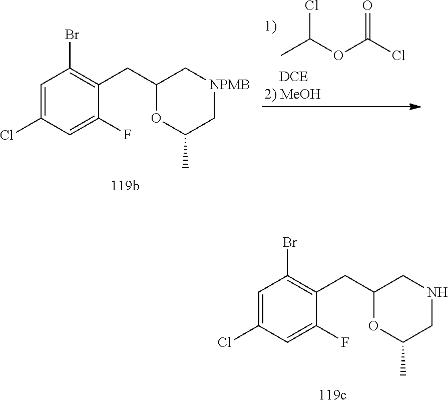

To the solution of 119b (a single diastereoisomer)(331 mg; 0.748 mmol) in DCE (3.4 mL) 1-chloroethyl carbonochloridate (323 μL; 2.990 mmol) was added dropwise and this mixture was then stirred at 80° C. for 2 hours. The reaction progress was monitored by LC-MS. After this time, the solvent was evaporated in vacuo and the residue was dissolved in MeOH (5 mL) and whole was stirred at 75° C. for 1 hour. Then the solvent was evaporated in vacuo and the crude product was used to the next step without any additional purification.

ESI-MS m/z for C₁₂H₁₅BrClFNO found 321.8/323.8 [M+H]⁺; Rₜ=1.02 min (Method C)

### Step 4.

### Synthesis of tert-butyl (6S)-2-(2-bromo-4-chloro-6-fluorobenzyl)-6-methylmorpholine-4-carboxylate (119d)

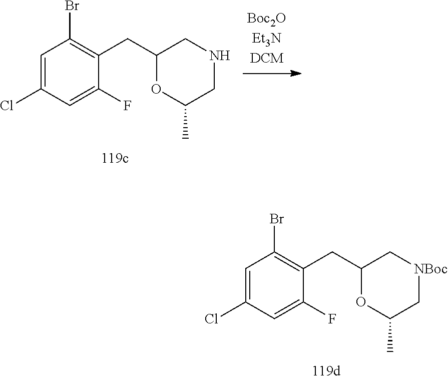 [SMILES: C[C@H]1CNCC(Cc2c(F)cc(Cl)cc2Br)O1 ; C[C@H]1CN(C(=O)OC(C)(C)C)CC(Cc2c(F)cc(Cl)cc2Br)O1]

To the solution of 119c (the crude product) in DCM (7.5 mL) Boc₂O (245 mg; 1.12 mmol) and Et₃N (0.16 mL; 1.12 mmol) were added and this mixture was then stirred at room temperature overnight. The reaction progress was monitored by LC-MS. When analysis indicated completion of the reaction, the reaction mixture was diluted with DCM and quenched with water. An aqueous phase (pH=8) was extracted with DCM. The combined organic phases were dried over anhydrous Na₂SO₄, filtered and concentrated in vacuo and the residue was purified by flash column chromatography on silica (hexane/AcOEt, 100:0 to 20:80, v/v, 15 min, 25 mL/min). The crude product was then used to the next step without further purification.

ESI-MS m/z for C₁₂H₁₅BrClFNO found 321.8/323.8 [M+H-Boc]⁺; Rₜ=2.01 min (Method C); ¹H NMR (400 MHz, CDCl₃) δ 7.42-7.38 (m, 1H), 7.08-7.02 (m, 1H), 3.91-3.78 (m, 2H), 3.67-3.59 (m, 1H), 3.53-3.42 (m, 1H), 3.06-2.98 (m, 1H), 2.94-2.85 (m, 1H), 2.67-2.40 (m, 2H), 1.43 (s, 9H), 1.14 (d, J=6.2 Hz, 3H).

### Step 5.

### Synthesis of tert-butyl (6S)-2-(4-chloro-2-fluoro-6-(4,4,5,5-tetramethyl-1,3,2-dioxaborolan-2-yl)benzyl)-6-methylmorpholine-4-carboxylate (119e)

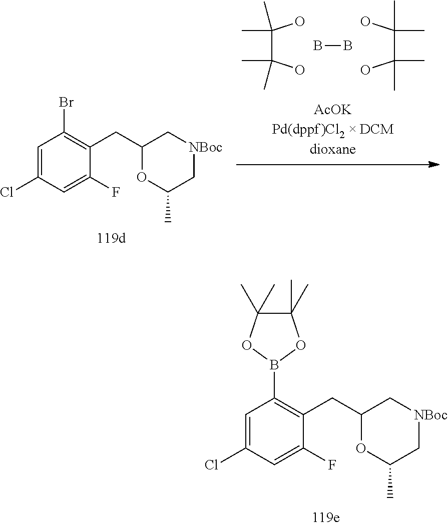

In a Schlenk flask was placed 119d (the crude product), bis(pinacolato)diboron (383 mg; 1.500 mmol), AcOK (142 mg; 2.240 mmol) and Pd(dppf)Cl₂×DCM (31 mg; 0.037 mmol) and dioxane (3.8 mL). The reaction mixture was intensively flushed with Ar. The mixture was capped and stirred at 100° C. overnight. The reaction progress was monitored by LC-MS. When analysis indicated completion of the reaction, the reaction mixture was concentrated in vacuo. The residue was then purified by flash column chromatography on silica (hexane/AcOEt, 100:0 to 30:70, v/v, 20 min, 20 mL/min). The crude product was used to the next step without any additional purification.

ESI-MS m/z for C₁₈H₂₇BClFNO₃ found 370.0/372.0 [M+H-Boc]⁺; Rₜ=2.24 min (Method C)

### Step 6.

### Synthesis of tert-butyl (6S)-2-(4-chloro-2-fluoro-6-(6-((4-methyl-2,6-dioxopiperazin-1-yl)methyl)pyrrolo[2,1-f][1,2,4]triazin-4-yl)benzyl)-6-methylmorpholine-4-carboxylate (119f)

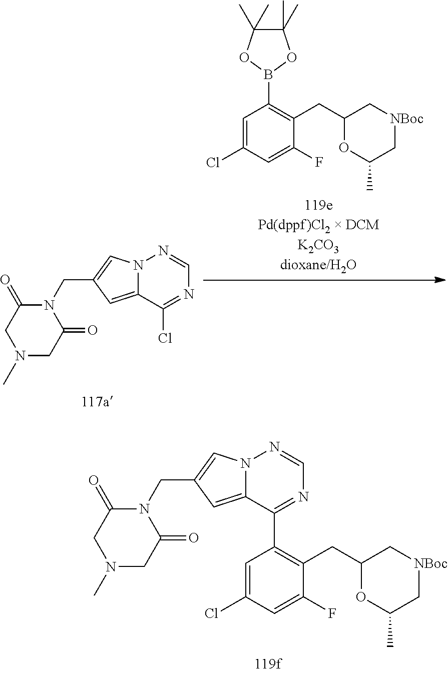 [SMILES: C[C@H]1CN(C(=O)OC(C)(C)C)CC(Cc2c(F)cc(Cl)cc2B2OC(C)(C)C(C)(C)O2)O1 ; CN1CC(=O)N(Cc2cc3c(Cl)ncnn3c2)C(=O)C1 ; C[C@H]1CN(C(=O)OC(C)(C)C)CC(Cc2c(F)cc(Cl)cc2-c2ncnn3cc(CN4C(=O)CN(C)CC4=O)cc23)O1]

The title compound (119f) was obtained from 117a′ (62 mg; 0.213 mmol) and from 119e (100 mg; 0.213 mmol) according to the General Procedure IV in 4% yield (5 mg; 0.008 mmol). The crude product was purified by flash column chromatography on silica (DCM/MeOH, 100:0 to 80:20, v/v, 30 min, 20 mL/min).

ESI-MS m/z for C₂₉H₃₅ClFN₆O₅ found 601.2/603.2 [M+H]⁺; Rₜ=1.75 min (Method C)

### Step 7.

### Synthesis of 1-((4-(5-chloro-3-fluoro-2-(((6S)-6-methylmorpholin-2-yl)methyl)phenyl)pyrrolo[2,1-f][1,2,4]triazin-6-yl)methyl)-4-methylpiperazine-2,6-dione hydrochloride (119)

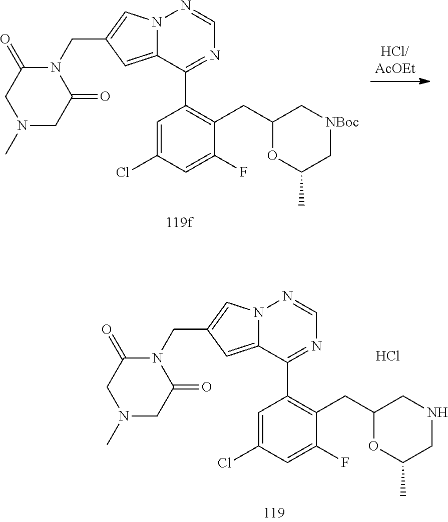 [SMILES: C[C@H]1CN(C(=O)OC(C)(C)C)CC(Cc2c(F)cc(Cl)cc2-c2ncnn3cc(CN4C(=O)CN(C)CC4=O)cc23)O1 ; C[C@H]1CNCC(Cc2c(F)cc(Cl)cc2-c2ncnn3cc(CN4C(=O)CN(C)CC4=O)cc23)O1 ; Cl]

The title compound (119) was obtained as a single isomer as a hydrochloride salt from 119f (5 mg; 0.0083 mmol) according to the General Procedure IIIa in 78% yield (3.5 mg; 0.0065 mmol). The crude product was purified by preparative reversed-phase column chromatography (C-18, water+10‰ HCl (36%)/MeCN, 95:5 to 5:95, 30 min, 20 mL/min, Rₜ=13.76 min).

ESI-MS m/z for C₂₄H₂₇ClFN₆O₃ found 501.1/503.1 [M+H]⁺; Rₜ=1.02 min (Method C); ¹H NMR (400 MHz, Methanol-d₄) δ 8.54 (s, 1H), 8.15 (d, J=1.3 Hz, 1H), 7.50 (d, J=9.1 Hz, 2H), 6.85 (d, J=1.3 Hz, 1H), 5.12 (s, 2H), 4.28 (s, 4H), 3.90-3.79 (m, 1H), 3.61-3.50 (m, 1H), 3.22-3.03 (m, 4H), 2.99 (s, 3H), 2.70-2.63 (m, 1H), 2.57-2.47 (m, 1H), 0.79 (d, J=6.2 Hz, 3H); ¹⁹F NMR (376 MHz, Methanol-d₄) δ −112.28 (d, J=9.3 Hz).

### Example 120

### Synthesis of 3-((4-(5-chloro-3-fluoro-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione hydrochloride (120)

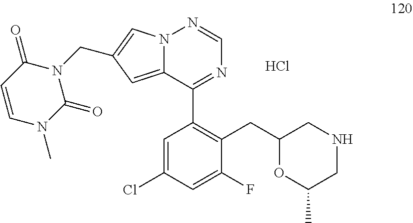 [SMILES: C[C@H]1CNCC(Cc2c(F)cc(Cl)cc2-c2ncnn3cc(Cn4c(=O)ccn(C)c4=O)cc23)O1 ; Cl]

The title compound (120) was obtained as a hydrochloride salt as a single isomer in 13% overall yield in a similar way to Example 116 with the exception that, in the first step of the synthesis, the compound 119b (a single diastereoisomer) was used instead of the compound 103c, and in the last step of the synthesis, the crude product was purified by preparative reversed-phase column chromatography (C-18, water+1‰ HCl (36%)/MeCN, 95:5 to 5:95, 30 min, 20 mL/min, Rₜ=14.15 min).

ESI-MS m/z for C₂₄H₂₅ClFN₆O₃ found 499.4/501.4 [M+H]⁺; Rₜ=0.96 min (Method C); ¹H NMR (400 MHz, Methanol-d₄) δ 8.62 (s, 1H), 8.31 (d, J=1.4 Hz, 1H), 7.60-7.54 (m, 3H), 7.04 (d, J=1.4 Hz, 1H), 5.73 (d, J=7.8 Hz, 1H), 5.27-5.18 (m, 2H), 3.90-3.81 (m, 1H), 3.59-3.48 (m, 1H), 3.37 (s, 3H), 3.28-3.20 (m, 1H), 3.19-3.01 (m, 3H), 2.70-2.59 (m, 1H), 2.52-2.42 (m, 1H), 0.64 (d, J=6.3 Hz, 3H); ¹⁹F NMR (376 MHz, Methanol-d₄) δ −111.92 (d, J=9.5 Hz).

### Example 121

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)pyrimidine-2,4(1H,3H)-dione dihydrochloride (121)

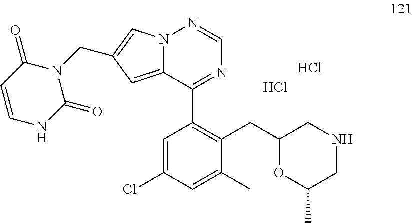 [SMILES: Cc1cc(Cl)cc(-c2ncnn3cc(Cn4c(=O)cc[nH]c4=O)cc23)c1CC1CNC[C@H](C)O1 ; Cl ; Cl]

The title compound (121) was obtained as a dihydrochloride salt as a single isomer in 6% overall yield in a similar way to Example 59 with the exception that, in the first step of the synthesis, the compound 27d′ was used instead of the compound 27d, in the second step of the synthesis, the compound 89a was used instead of 1-methylpyrimidine-2,4(1H,3H)-dione and in the last step of the synthesis, the crude product was purified by preparative reversed-phase column chromatography (C-18, water+0.3‰ HCl (36%)/MeCN, 99:1 to 20:80, 30 min, 20 mL/min, Rₜ=13.95 min).

ESI-MS m/z for C₂₄H₂₆ClN₆O₃ found 481.4/483.4 [M+H]⁺; Rₜ=0.91 min (Method A); ¹H NMR (700 MHz, Methanol-d₄) δ 8.53 (s, 1H), 8.15 (d, J=1.2 Hz, 1H), 7.50 (d, J=2.1 Hz, 1H), 7.40 (d, J=2.1 Hz, 1H), 7.38 (d, J=7.6 Hz, 1H), 6.77 (s, 1H), 5.70 (d, J=7.6 Hz, 1H), 5.19-5.16 (m, 2H), 4.02-3.99 (m, 1H), 3.80-3.77 (m, 1H), 3.12-3.06 (m, 3H), 2.99-2.96 (m, 1H), 2.88-2.84 (m, 2H), 2.47 (s, 3H), 0.95 (d, J=6.8 Hz, 3H).

### Example 122

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione dihydrochloride (122)

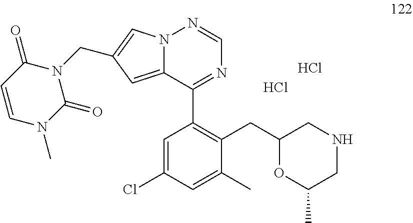 [SMILES: Cc1cc(Cl)cc(-c2ncnn3cc(Cn4c(=O)ccn(C)c4=O)cc23)c1CC1CNC[C@H](C)O1 ; Cl ; Cl]

### Step 1.

### Synthesis of tert-butyl (6S)-2-(4-chloro-2-(6-((2,6-dioxo-3,6-dihydropyrimidin-1(2H)-yl)methyl)pyrrolo[2,1-f][1,2,4]triazin-4-yl)-6-methylbenzyl)-6-methylmorpholine-4-carboxylate and tert-butyl (6S)-2-(2-(6-((3-(tert-butoxycarbonyl)-2,6-dioxo-3,6-dihydropyrimidin-1(2H)-yl)methyl)pyrrolo[2,1-f][1,2,4]triazin-4-yl)-4-chloro-6-methylbenzyl)-6-methylmorpholine-4-carboxylate (122a and 122a′)

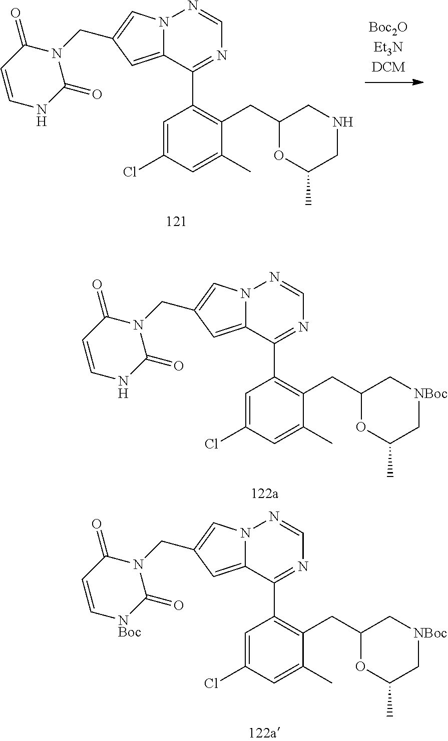 [SMILES: Cc1cc(Cl)cc(-c2ncnn3cc(Cn4c(=O)cc[nH]c4=O)cc23)c1CC1CNC[C@H](C)O1 ; Cc1cc(Cl)cc(-c2ncnn3cc(Cn4c(=O)cc[nH]c4=O)cc23)c1CC1CN(C(=O)OC(C)(C)C)C[C@H](C)O1 ; Cc1cc(Cl)cc(-c2ncnn3cc(Cn4c(=O)ccn(C(=O)OC(C)(C)C)c4=O)cc23)c1CC1CN(C(=O)OC(C)(C)C)C[C@H](C)O1]

To the solution of 121 (as a free amine)(112 mg; 0.233 mmol) in DCM (4.5 mL) Boc₂O (52 mg; 0.233 mmol) and Et₃N (0.065 mL; 0.466 mmol) were added and this mixture was then stirred at room temperature overnight. The reaction progress was monitored by LC-MS. When analysis indicated completion of the reaction, the reaction mixture was concentrated in vacuo and the residue was purified by flash column chromatography on silica (hexane/AcOEt, 100:0 to 0:100, v/v, 35 min, 13 mL/min). Compound 122a was obtained in 52% yield (71 mg; 0.122 mmol) and the compound 122a′ was obtained in 16% yield (26 mg; 0.038 mmol).

For 122a: ESI-MS m/z for C₂₉H₃₄ClN₆O₅ found 581.4/583.4 [M+H]⁺; Rₜ=1.50 min (Method A);

For 122a′: ESI-MS m/z for C₃₄H₄₂ClN₆O₇ found 681.2/683.2 [M+H]⁺; Rₜ=1.94 min (Method A)

### Step 2.

### Synthesis of tert-butyl (6S)-2-(4-chloro-2-methyl-6-(6-((3-methyl-2,6-dioxo-3,6-dihydropyrimidin-1(2H)-yl)methyl)pyrrolo[2,1-f][1,2,4]triazin-4-yl)benzyl)-6-methylmorpholine-4-carboxylate (122b)

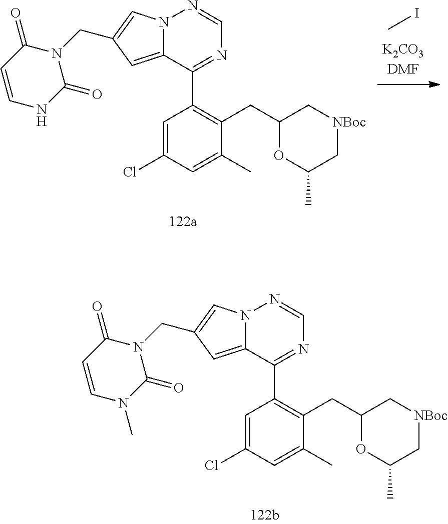

To the solution of 122a (71 mg; 0.122 mmol) in dry DMF (4.6 mL) K₂CO₃ (34 mg; 0.244 mmol) and methyl iodide (7.7 μL; 0.122 mmol) were added. The resulting mixture was then stirred at room temperature for 1 hour. The reaction progress was monitored by LC-MS. When analysis indicated completion of the reaction, the reaction mixture was concentrated in vacuo and the residue was purified by flash column chromatography on silica (hexane/AcOEt, 100:0 to 0:100, v/v, 30 min, 13 mL/min). Compound 122b was obtained in 85% yield (62 mg; 0.104 mmol).

ESI-MS m/z for C₃₀H₃₆ClN₆O₅ found 595.4/597.4 [M+H]⁺; Rₜ=1.64 min (Method A)

### Step 3.

### Synthesis of 3-((4-(5-chloro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione dihydrochloride (122)

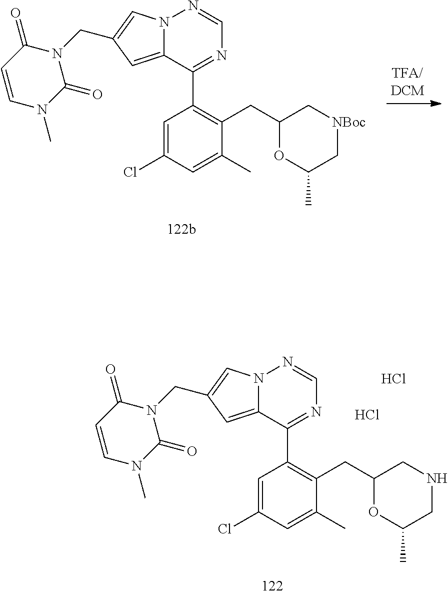 [SMILES: Cc1cc(Cl)cc(-c2ncnn3cc(Cn4c(=O)ccn(C)c4=O)cc23)c1CC1CN(C(=O)OC(C)(C)C)C[C@H](C)O1 ; Cc1cc(Cl)cc(-c2ncnn3cc(Cn4c(=O)ccn(C)c4=O)cc23)c1CC1CNC[C@H](C)O1 ; Cl ; Cl]

The title compound (122) was obtained as a dihydrochloride salt from 122b (62 mg; 0.104 mmol) according to the General Procedure IIIb in 42% yield (25 mg; 0.044 mmol). The crude product was purified by preparative reversed-phase column chromatography (C-18, water+0.3‰ HCl (36%)/MeCN, 99:1 to 20:80, 30 min, 20 mL/min, Rₜ=14.22 min).

ESI-MS m/z for C₂₅H₂₈ClN₆O₃ found 495.4/497.4 [M+H]⁺; Rₜ=0.96 min (Method A); ¹H NMR (700 MHz, Methanol-d₄) δ 8.49 (s, 1H), 8.05 (d, J=1.5 Hz, 1H), 7.54 (d, J=7.8 Hz, 1H), 7.47 (d, J=1.9 Hz, 1H), 7.37 (d, J=2.2 Hz, 1H), 6.66 (d, J=1.5 Hz, 1H), 5.72 (d, J=7.8 Hz, 1H), 5.19 (s, 2H), 4.04-4.00 (m, 1H), 3.82-3.78 (m, 1H), 3.36 (s, 3H), 3.17-3.13 (m, 1H), 3.11-3.06 (m, 2H), 2.98-2.94 (m, 1H), 2.89-2.85 (m, 2H), 2.46 (s, 3H), 0.94 (d, J=6.8 Hz, 3H).

### Example 123

### Synthesis of 3-((2-chloro-4-(5-chloro-1-((4-fluoropiperidin-4-yl)methyl)-1H-indol-7-yl)pyrrolo[2,1-f][1,2,4]triazin-6-yl)methyl)pyrimidine-2,4(1H,3H)-dione hydrochloride (123)

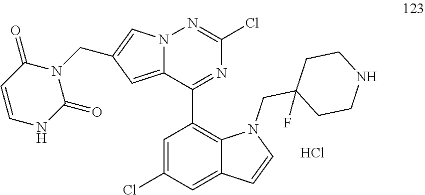 [SMILES: O=c1cc[nH]c(=O)n1Cc1cc2c(-c3cc(Cl)cc4ccn(CC5(F)CCNCC5)c34)nc(Cl)nn2c1 ; Cl]

The title compound (123) was obtained as a hydrochloride salt in 36% overall yield in a similar way to Example 21 with the exception that, in the first step of the synthesis, the compound 123b (the synthesis of this compound was described below) was used instead of the compound 13b, and in the last step of the synthesis, the crude product was purified by preparative reversed-phase column chromatography (C-18, water+1‰ HCl (36%)/MeCN, 98:2 to 30:70, 30 min, 20 mL/min, Rₜ=16.30 min).

ESI-MS m/z for C₂₅H₂₃Cl₂FN₇O₂ found 542.2/544.2 [M+H]⁺; Rₜ=1.08 min (Method A); ¹H NMR (700 MHz, Methanol-d₄) δ 8.07 (d, J=1.5 Hz, 1H), 7.86 (d, J=2.1 Hz, 1H), 7.56 (d, J=2.1 Hz, 1H), 7.40 (d, J=7.6 Hz, 1H), 7.38 (d, J=3.3 Hz, 1H), 6.90 (d, J=1.5 Hz, 1H), 6.72 (d, J=3.2 Hz, 1H), 5.72 (d, J=7.6 Hz, 1H), 5.18 (s, 2H), 4.62-4.57 (m, 2H), 3.17-3.13 (m, 2H), 2.89-2.83 (m, 3H), 1.71-1.60 (m, 3H), 1.51-1.47 (m, 2H).

### Synthesis of tert-butyl 3-((2,4-dichloropyrrolo[2,1-f][1,2,4]triazin-6-yl)methyl)-2,4-dioxo-3,4-dihydropyrimidine-1(2H)-carboxylate (123b)

### Step 1.

### Synthesis of (2,4-dichloropyrrolo[2,1-f][1,2,4]triazin-6-yl)methanol (123a)

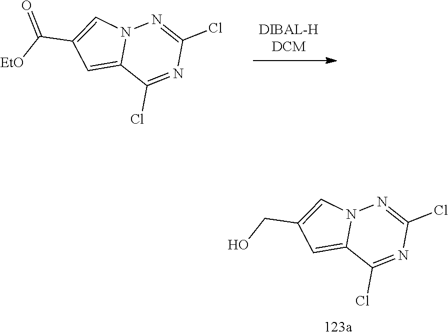 [SMILES: CCOC(=O)c1cc2c(Cl)nc(Cl)nn2c1 ; OCc1cc2c(Cl)nc(Cl)nn2c1]

To a stirred solution of ethyl 2,4-dichloropyrrolo[2,1-f][1,2,4]triazine-6-carboxylate (379 mg; 1.46 mmol) in dry DCM (10 mL) DIBAL-H (1 M in DCM; 7.29 mL; 7.29 mmol) was added dropwise at −78° C. Then the reaction mixture was stirred at this temperature for 1 hour. The reaction progress was monitored by LC-MS. Next water was added dropwise to the reaction mixture (without stirring) and this mixture was transferred to the separatory funnel and to this mixture DCM was added. The phases were separated and an aqueous one was extracted with DCM (3×30 mL). The combined organic solutions were dried over anhydrous Na₂SO₄, filtered and concentrated in vacuo and the crude product was purified by flash column chromatography on silica (hexane/AcOEt, 100:0 to 0:100, v/v, 15 min, 18 mL/min). Compound 123a was obtained as a white solid in 60% yield (191 mg; 0.88 mmol).

ESI-MS m/z for C₇H₆Cl₂N₃O found 217.9/219.9 [M+H]⁺; Rₜ=0.93 min (Method A)

### Step 2.

### Synthesis of tert-butyl 3-((2,4-dichloropyrrolo[2,1-f][1,2,4]triazin-6-yl)methyl)-2,4-dioxo-3,4-dihydropyrimidine-1(2H)-carboxylate (123b)

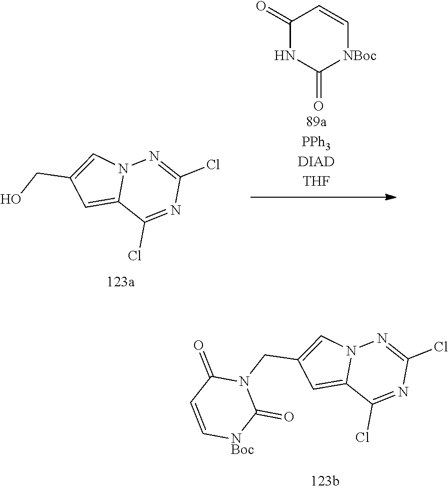 [SMILES: CC(C)(C)OC(=O)n1ccc(=O)[nH]c1=O ; OCc1cc2c(Cl)nc(Cl)nn2c1 ; CC(C)(C)OC(=O)n1ccc(=O)n(Cc2cc3c(Cl)nc(Cl)nn3c2)c1=O]

To a cooled to −10° C. solution of 123a (191 mg; 0.876 mmol), 89a (204 mg; 0.964 mmol) and PPh₃ (276 mg; 1.050 mmol) in THE (10 mL) DIAD (228 μL; 1.140 mmol) was added dropwise. The resulting mixture was stirred at room temperature for 1 hour. Then the reaction mixture was concentrated in vacuo and the residue was purified by flash column chromatography on silica (column: 12 g; hexane/AcOEt, 100:0 to 0:100, v/v, 30 min, 30 mL/min). Compound 123b was obtained in 99% yield (356 mg; 0.867 mmol).

ESI-MS m/z for C₁₆H₁₆Cl₂N₅O₄ found 411.9/413.9 [M+H]⁺; Rₜ=1.53 min (Method A)

### Example 124

### Synthesis of (R)-3-((4-(5-chloro-3-methyl-2-(morpholin-2-ylmethyl)phenyl)pyrrolo[2,1-f][1,2,4]triazin-6-yl)methyl)pyrimidine-2,4(1H,3H)-dione hydrochloride (124)

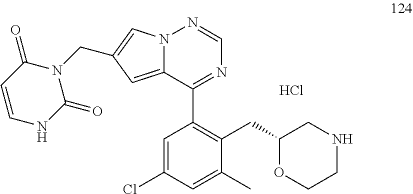 [SMILES: Cc1cc(Cl)cc(-c2ncnn3cc(Cn4c(=O)cc[nH]c4=O)cc23)c1C[C@@H]1CNCCO1 ; Cl]

The title compound (124) was obtained as a hydrochloride salt as a single enantiomer in 12% overall yield in a similar way to Example 82 with the exception that, in the fifth step of the synthesis, the compound 89b was used instead of the compound 59b, and in the last step of the synthesis, the crude product was purified by preparative reversed-phase column chromatography (C-18, water+0.3‰ HCl (36%)/MeCN, 99:1 to 20:80, 30 min, 20 mL/min, Rₜ=12.50 min).

ESI-MS m/z for C₂₃H₂₄ClN₆O₃ found 467.1/469.1 [M+H]⁺; Rₜ=0.92 min (Method A); ¹H NMR (700 MHz, Methanol-d₄) δ 8.75 (s, 1H), 8.59 (d, J=1.4 Hz, 1H), 7.64 (d, J=2.3 Hz, 1H), 7.51 (d, J=2.3 Hz, 1H), 7.43 (d, J=7.6 Hz, 1H), 7.24 (d, J=1.4 Hz, 1H), 5.73 (d, J=7.6 Hz, 1H), 5.25 (s, 2H), 3.81-3.69 (m, 2H), 3.59-3.49 (m, 1H), 3.32-3.27 (m, 1H), 3.16-3.10 (m, 1H), 3.07-3.00 (m, 1H), 2.97-2.89 (m, 1H), 2.87-2.74 (m, 2H), 2.53 (s, 3H).

### Example 125

### Synthesis of (R)-2-(3-((4-(5-chloro-3-methyl-2-(morpholin-2-ylmethyl)phenyl)pyrrolo[2,1-f][1,2,4]triazin-6-yl)methyl)-2,4-dioxo-3,4-dihydropyrimidin-1(2H)-yl)acetic acid hydrochloride (125)

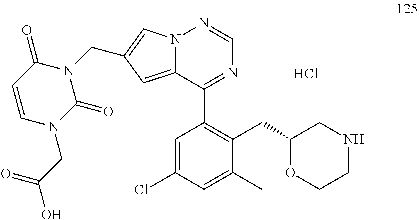 [SMILES: Cc1cc(Cl)cc(-c2ncnn3cc(Cn4c(=O)ccn(CC(=O)O)c4=O)cc23)c1C[C@@H]1CNCCO1 ; Cl]

The title compound (125) was obtained as a hydrochloride salt as a single enantiomer in 32% overall yield in a similar way to Example 89 with the exception that, in the fourth step of the synthesis, tert-butyl bromoacetate was used instead of 2-iodopropane, and in the last step of the synthesis, the crude product was purified by preparative reversed-phase column chromatography (C-18, water+0.3‰ HCl (36%)/MeCN, 99:1 to 40:60, 30 min, 20 mL/min, Rₜ=18.60 min).

ESI-MS m/z for C₂₅H₂₆ClN₆O₅ found 525.3/527.3 [M+H]⁺; Rₜ=0.95 min (Method A); ¹H NMR (700 MHz, Methanol-d₄) δ 8.48 (s, 1H), 8.05 (d, J=1.5 Hz, 1H), 7.55 (d, J=7.9 Hz, 1H), 7.48-7.45 (m, 1H), 7.36 (d, J=2.3 Hz, 1H), 6.68 (d, J=1.5 Hz, 1H), 5.78 (d, J=7.9 Hz, 1H), 5.20 (s, 2H), 4.57-4.52 (m, 2H), 3.72-3.64 (m, 2H), 3.45-3.38 (m, 1H), 3.13-3.04 (m, 2H), 3.04-2.97 (m, 1H), 2.96-2.88 (m, 2H), 2.75-2.69 (m, 1H), 2.47 (s, 3H).

### Example 126

### Synthesis of 3-((4-(3,5-dichloro-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione hydrochloride (126)

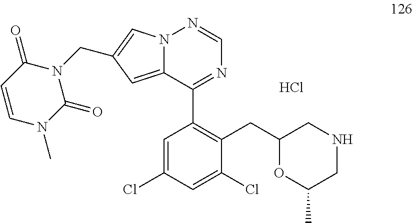 [SMILES: C[C@H]1CNCC(Cc2c(Cl)cc(Cl)cc2-c2ncnn3cc(Cn4c(=O)ccn(C)c4=O)cc23)O1 ; Cl]

The title compound (126) was obtained as a hydrochloride salt as a single isomer in 8% overall yield in a similar way to Example 116 with the exception that, in the first step of the synthesis, (6S)-2-(2-bromo-4,6-dichlorobenzyl)-4-(4-methoxybenzyl)-6-methylmorpholine (this compound was obtained like the compound 119b with the exception that, in the first step of the synthesis, 1-bromo-2-(bromomethyl)-3,5-dichlorobenzene was used instead of 1-bromo-2-(bromomethyl)-5-chloro-3-fluorobenzene) was used instead of the compound 103c, and in the last step of the synthesis, the crude product was purified by preparative reversed-phase column chromatography (C-18, water+1‰ HCl (36%)/MeCN, 95:5 to 5:95, 30 min, 20 mL/min, Rₜ=14.26 min).

ESI-MS m/z for C₂₄H₂₅Cl₂N₆O₃ found 515.1/517.1 [M+H]⁺; Rₜ=1.02 min (Method C); ¹H NMR (400 MHz, Methanol-d₄) δ 8.57 (s, 1H), 8.21 (d, J=1.4 Hz, 1H), 7.82 (d, J=2.2 Hz, 1H), 7.64 (d, J=2.2 Hz, 1H), 7.57 (d, J=7.8 Hz, 1H), 6.92-6.87 (m, 1H), 5.74 (d, J=7.8 Hz, 1H), 5.32-5.15 (m, 2H), 3.95-3.85 (m, 1H), 3.54-3.43 (m, 1H), 3.38 (s, 3H), 3.36-3.18 (m, 3H), 3.08-3.02 (m, 1H), 2.72-2.62 (m, 1H), 2.49-2.40 (m, 1H), 0.58 (d, J=6.2 Hz, 3H).

### Example 127

### Synthesis of 3-((4-(5-chloro-1-((4-fluoropiperidin-4-yl)methyl)-1H-indol-7-yl)pyrrolo[2,1-f][1,2,4]triazin-6-yl)methyl)-1-isopropylpyrimidine-2,4(1H,3H)-dione hydrochloride (127)

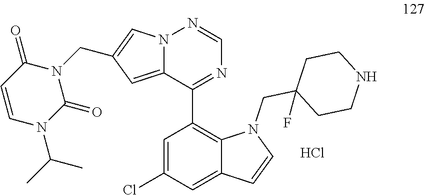 [SMILES: CC(C)n1ccc(=O)n(Cc2cc3c(-c4cc(Cl)cc5ccn(CC6(F)CCNCC6)c45)ncnn3c2)c1=O ; Cl]

The title compound (127) was obtained as a hydrochloride salt in 10% overall yield in a similar way to Example 89 with the exception that, in the third step of the synthesis, the compound 10c was used instead of the compound 2a, and in the last step of the synthesis, the crude product was purified by preparative reversed-phase column chromatography (C-18, water+0.3‰ HCl (36%)/MeCN, 98:2 to 30:70, 30 min, 20 mL/min, Rₜ=18.80 min).

ESI-MS m/z for C₂₈H₃₀ClFN₇O₂ found 550.1/552.1 [M+H]⁺; Rₜ=1.12 min (Method A); ¹H NMR (700 MHz, Methanol-d₄) δ 8.57 (s, 1H), 8.07 (d, J=1.5 Hz, 1H), 7.83 (d, J=2.1 Hz, 1H), 7.68 (d, J=8.0 Hz, 1H), 7.45 (d, J=2.0 Hz, 1H), 7.39-7.34 (m, 1H), 6.76 (d, J=1.5 Hz, 1H), 6.71 (d, J=3.3 Hz, 1H), 5.80 (d, J=8.0 Hz, 1H), 5.22 (s, 2H), 4.43-4.36 (m, 2H), 3.38-3.32 (m, 1H), 3.18-3.12 (m, 2H), 2.93-2.83 (m, 2H), 1.69-1.56 (m, 2H), 1.49-1.42 (m, 2H), 1.36-1.31 (m, 6H).

### Example 128

### Synthesis of 3-((4-(5-fluoro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione hydrochloride (128)

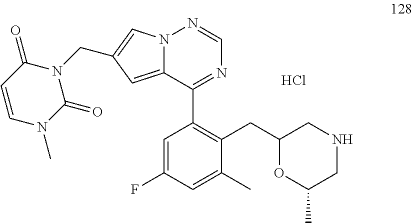 [SMILES: Cc1cc(F)cc(-c2ncnn3cc(Cn4c(=O)ccn(C)c4=O)cc23)c1CC1CNC[C@H](C)O1 ; Cl]

The title compound (128) was obtained as a hydrochloride salt in 19% overall yield in a similar way to Example 116 with the exception that, in the first step of the synthesis, the compound 128c (the synthesis of this compound was described below) was used instead of the compound 103c, and in the last step of the synthesis, the crude product was purified by preparative reversed-phase column chromatography (C-18, water+1‰ HCl (36%)/MeCN, 98:2 to 30:70, 30 min, 20 mL/min, Rₜ=14.63 min).

ESI-MS m/z for C₂₅H₂₈FN₆O₃ found 479.4/481.4 [M+H]⁺; Rₜ=0.88 min (Method A); ¹H NMR (700 MHz, D₂O) δ 8.51 (s, 1H), 8.13 (s, 1H), 7.60 (d, J=7.8 Hz, 1H), 7.29-7.26 (m, 1H), 7.15-7.11 (m, 1H), 6.81 (s, 1H), 5.86 (d, J=7.8 Hz, 1H), 5.24-5.17 (m, 2H), 3.77-3.70 (m, 1H), 3.50-3.43 (m, 1H), 3.39 (s, 3H), 3.27-3.19 (m, 2H), 3.12-3.08 (m, 1H), 3.05-2.98 (m, 1H), 2.65-2.59 (m, 1H), 2.48-2.43 (m, 4H), 0.56 (d, J=5.3 Hz, 3H).

### Synthesis of (6S)-2-(2-bromo-4-fluoro-6-methylbenzyl)-4-(4-methoxybenzyl)-6-methylmorpholine (128c)

### Step 1.

### Synthesis of 1-bromo-2-(bromomethyl)-5-fluoro-3-methylbenzene (128a)

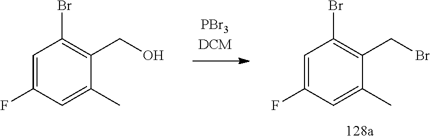

To a cooled to 0° C. solution of (2-bromo-4-fluoro-6-methylphenyl)methanol (490 mg; 2.24 mmol) in dry DCM (8 mL) PBr₃ (428 μL; 4.47 mmol) was added dropwise under an Argon atmosphere. The resulting mixture was allowed to warm to room temperature and stirred for 3 hours. The reaction progress was monitored by LC-MS (no ionization was observed). When analysis indicated completion of the reaction, the reaction mixture was cooled to 0° C., diluted with DCM and quenched with a saturated aqueous solution of NaHCO₃. The layers were separated and an aqueous one was then extracted with DCM. The combined organic layers were dried over anhydrous Na₂SO₄, filtered and concentrated in vacuo. Compound 128a was obtained in 78% yield (488 mg; 1.74 mmol).

### Step 2.

### Synthesis of (6S)-2-(2-bromo-4-fluoro-6-methylbenzyl)-4-(4-methoxybenzyl)-6-methylmorpholin-3-one (128b)

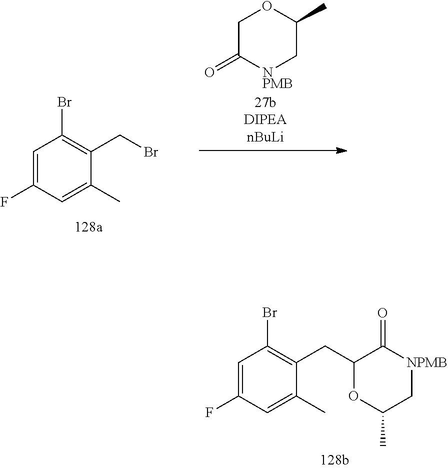

To the cooled to −78° C. solution of DIPEA (0.22 mL; 1.56 mmol) in dry THF (1.5 mL) nBuLi (2.5 M in hexane; 624 μL; 1.56 mmol) was added and the reaction mixture was warmed to room temperature and stirred at this temperature for 15 minutes. Then the reaction mixture was cooled to −78° C. and to this mixture a solution of 27b (334 mg; 1.42 mmol) in THE (2 mL) was added and a whole was stirred at this temperature for 1 hour. Then to the reaction mixture a solution of 128a (400 mg; 1.42 mmol) in dry THE (3 mL) was added and stirred at −78° C. for 30 minutes, then the reaction mixture was slowly warmed to room temperature and stirred overnight. After this time, the reaction mixture was quenched with a saturated solution of NH₄Cl and extracted with DCM (3×). The combined organic solutions were dried over anhydrous MgSO₄, filtered and concentrated in vacuo. Then the crude product was used to the next step without any additional purification.

### Step 3.

### Synthesis of (6S)-2-(2-bromo-4-fluoro-6-methylbenzyl)-4-(4-methoxybenzyl)-6-methylmorpholine (128c)

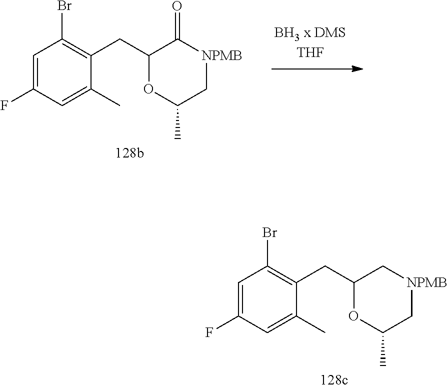

To the cooled to 0° C. solution of 128b (the crude product) in dry THF (7.6 mL) BH₃×DMS (2.23 mL; 4.47 mmol) was added dropwise under an Argon atmosphere and this mixture was then stirred at 70° C. overnight. The reaction progress was monitored by LC-MS. When analysis indicated completion of the reaction, the reaction mixture was cooled to 0° C. and to this mixture 2 M HCl was added and a whole was stirred at 70° C. for 1 hour. Then to this solution 4 M NaOH were added (pH=8) and a whole was extracted with AcOEt. The combined organic phases were dried over anhydrous Na₂SO₄, filtered, and concentrated in vacuo. The crude product was used to the next step without additional purification. Compound 128c was obtained in 99% yield (per two steps)(594 mg; 1.41 mmol).

ESI-MS m/z for C₂₁H₂₆BrFNO₂ found 422.0/423.9 [M+H]⁺; Rₜ=1.33 min (Method A)

### Example 129

### Synthesis of 1-methyl-3-((4-(2-methyl-3-(piperidin-3-yloxy)-6-(trifluoromethyl)pyridin-4-yl)pyrrolo[2,1-f][1,2,4]triazin-6-yl)methyl)pyrimidine-2,4(1H,3H)-dione hydrochloride (129)

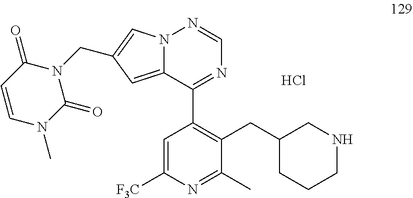 [SMILES: Cc1nc(C(F)(F)F)cc(-c2ncnn3cc(Cn4c(=O)ccn(C)c4=O)cc23)c1CC1CCCNC1 ; Cl]

### Step 1.

### Synthesis of 4-bromo-2-methyl-6-(trifluoromethyl)pyridin-3-ol (129a)

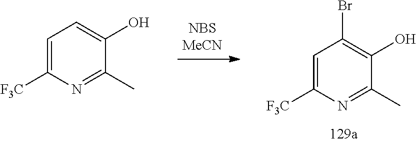

To the solution of 2-methyl-6-(trifluoromethyl)pyridin-3-ol (434 mg; 2.45 mmol) in MeCN (23 mL) NBS (485 mg; 2.70 mmol) was added at 0° C. and the resulting mixture was allowed to warm to room temperature and stirred at this temperature for 2 hours. The reaction progress was monitored by LC-MS. When analysis indicated completion of the reaction, to the reaction mixture Na₂S₂O₃ was added and extracted with AcOEt. The organic layer was dried over anhydrous Na₂SO₄, filtered and concentrated in vacuo. Compound 129a was obtained in 99% yield (617 mg; 2.42 mmol).

ESI-MS m/z for C₇H₆BrF₃NO found 255.9/257.9 [M+H]⁺; Rₜ=1.15 min (Method A); ¹H NMR (700 MHz, CDCl₃) δ 7.66 (s, 1H), 2.61 (s, 3H).

### Step 2.

### Synthesis of tert-butyl 3-((4-bromo-2-methyl-6-(trifluoromethyl)pyridin-3-yl)oxy)piperidine-1-carboxylate (129b)

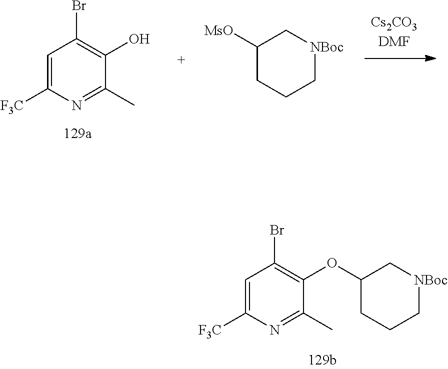 [SMILES: Cc1nc(C(F)(F)F)cc(Br)c1O ; CC(C)(C)OC(=O)N1CCCC(OS(C)(=O)=O)C1 ; Cc1nc(C(F)(F)F)cc(Br)c1OC1CCCN(C(=O)OC(C)(C)C)C1]

The solution of 129a (400 mg; 1.56 mmol), tert-butyl 3-((methylsulfonyl)oxy)piperidine-1-carboxylate (523 mg; 1.87 mmol) and Cs₂CO₃ (1017 mg; 3.12 mmol) in dry DMF (6 mL) was stirred under an Argon atmosphere at 70° C. overnight and then at 90° C. overnight. Then another portion of tert-butyl 3-((methylsulfonyl)oxy)piperidine-1-carboxylate (810 mg; 2.90 mmol) and Cs₂CO₃ (508 mg; 1.56 mmol) were added and a whole was stirred at 80° C. overnight and then at 50° C. for 2 days, then at 80° C. for 1 day. The reaction progress was monitored by LC-MS. When analysis indicated almost completion of the reaction, a whole was concentrated in vacuo. The crude product was then purified by flash column chromatography on silica (hexane/AcOEt, 100:0 to 60:40, v/v, 14 min, 30 mL/min). Compound 129b was obtained in 36% yield (251 mg; 0.57 mmol).

ESI-MS m/z for C₁₇H₂₃BrF₃N₂O₃ found 439.0/441.0 [M+H]⁺; Rₜ=1.92 min (Method A)

### Step 3.

### Synthesis of tert-butyl 3-((2-methyl-4-(4,4,5,5-tetramethyl-1,3,2-dioxaborolan-2-yl)-6-(trifluoromethyl)pyridin-3-yl)oxy)piperidine-1-carboxylate (129c)

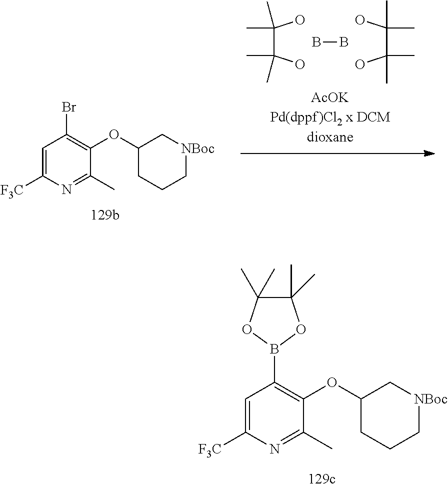

In a glass vial was placed 129b (223 mg; 0.508 mmol), bis(pinacolato)diboron (399 mg; 1.520 mmol), AcOK (101 mg; 1.020 mmol) and Pd(dppf)Cl₂×DCM (85 mg; 0.102 mmol) and dioxane (15 mL). The reaction mixture was intensively flushed with Ar. The mixture was capped and stirred at 100° C. overnight. The reaction progress was monitored by LC-MS. When analysis indicated completion of the reaction, the reaction mixture was concentrated in vacuo. The residue was then purified by flash column chromatography on silica (hexane/AcOEt, 100:0 to 60:40, v/v, 12 min, 35 mL/min). Compound 129c was obtained in 49% yield (122 mg; 0.249 mmol).

ESI-MS m/z for C₂₃H₃₅BF₃N₂O₅ found 487.1 [M+H]⁺; Rₜ=2.11 min (Method A)

### Step 4.

### Synthesis of tert-butyl 3-((4-(6-((2,6-dioxo-3,6-dihydropyrimidin-1(2H)-yl)methyl)pyrrolo[2,1-f][1,2,4]triazin-4-yl)-2-methyl-6-(trifluoromethyl)pyridin-3-yl)oxy)piperidine-1-carboxylate (129d)

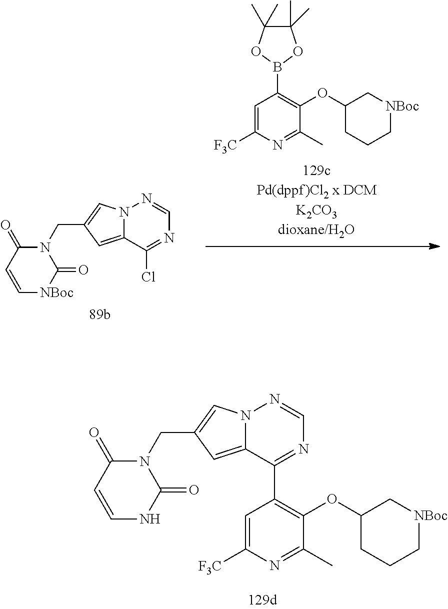 [SMILES: Cc1nc(C(F)(F)F)cc(B2OC(C)(C)C(C)(C)O2)c1OC1CCCN(C(=O)OC(C)(C)C)C1 ; CC(C)(C)OC(=O)n1ccc(=O)n(Cc2cc3c(Cl)ncnn3c2)c1=O ; Cc1nc(C(F)(F)F)cc(-c2ncnn3cc(Cn4c(=O)cc[nH]c4=O)cc23)c1OC1CCCN(C(=O)OC(C)(C)C)C1]

The title compound (129d) was obtained from 89b (88 mg; 0.234 mmol) and from 129c (76 mg; 0.156 mmol) according to the General Procedure IV in 43% yield (41 mg; 0.068 mmol). The crude product was purified by flash column chromatography on silica (hexane/AcOEt, 100:0 to 0:100, v/v, 12 min, then: 100% AcOEt, 10 min, 32 mL/min).

ESI-MS m/z for C₂₈H₃₁F₃N₇O₅ found 602.2 [M+H]⁺; Rₜ=1.47 min (Method A)

### Step 5.

### Synthesis of tert-butyl 3-((2-methyl-4-(6-((3-methyl-2,6-dioxo-3,6-dihydropyrimidin-1(2H)-yl)methyl)pyrrolo[2,1-f][1,2,4]triazin-4-yl)-6-(trifluoromethyl)pyridin-3-yl)oxy)piperidine-1-carboxylate (129e)

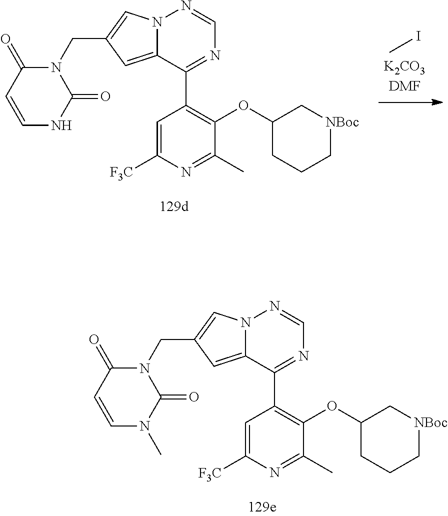

To the solution of 129d (41 mg; 0.068 mmol) in dry DMF (2 mL) K₂CO₃ (19 mg; 0.137 mmol) and methyl iodide (5 μL; 0.082 mmol) were added. The resulting mixture was stirred at room temperature for 2 hours. Then another portion of methyl iodide (5 μL; 0.082 mmol) was added and stirred at room temperature for 2 hours. The reaction progress was monitored by LC-MS. When analysis indicated completion of the reaction, the solvent was removed in vacuo and the residue was purified by flash column chromatography on silica (hexane/AcOEt, 100:0 to 0:100, v/v, 12 min, then: 100% AcOEt, 10 min, 14 mL/min). Compound 129e was obtained in 85% yield (36 mg; 0.058 mmol).

ESI-MS m/z for C₂₉H₃₃F₃N₇O₅ found 616.2 [M+H]⁺; Rₜ=1.57 min (Method A)

### Step 6.

### Synthesis of 1-methyl-3-((4-(2-methyl-3-(piperidin-3-yloxy)-6-(trifluoromethyl)pyridin-4-yl)pyrrolo[2,1-f][1,2,4]triazin-6-yl)methyl)pyrimidine-2,4(1H,3H)-dione hydrochloride (129)

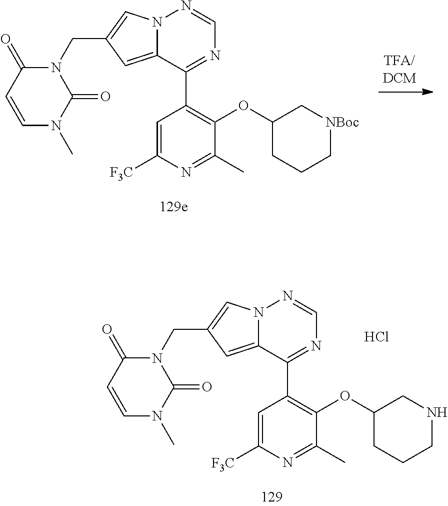 [SMILES: Cc1nc(C(F)(F)F)cc(-c2ncnn3cc(Cn4c(=O)ccn(C)c4=O)cc23)c1OC1CCCN(C(=O)OC(C)(C)C)C1 ; Cc1nc(C(F)(F)F)cc(-c2ncnn3cc(Cn4c(=O)ccn(C)c4=O)cc23)c1OC1CCCNC1 ; Cl]

The title compound (129) was obtained as a hydrochloride salt from 129e (36 mg; 0.058 mmol) according to the General Procedure IIIb in 84% yield (27 mg; 0.049 mmol). The crude product was purified by preparative reversed-phase column chromatography (C-18, water+0.30‰ HCl (36%)/MeCN, 99:1 to 50:50, 30 min, 20 mL/min, Rₜ=18.20 min).

ESI-MS m/z for C₂₄H₂₅F₃N₇O₃ found 516.4 [M+H]⁺; Rₜ=0.89 min (Method A); ¹H NMR (700 MHz, Methanol-d₄) δ 8.59 (s, 1H), 8.11 (d, J=1.4 Hz, 1H), 7.88 (s, 1H), 7.57 (d, J=7.8 Hz, 1H), 6.86 (d, J=1.5 Hz, 1H), 5.74 (d, J=7.8 Hz, 1H), 5.28-5.18 (m, 2H), 4.16-4.10 (m, 1H), 3.38 (s, 3H), 3.14-3.00 (m, 4H), 2.75 (s, 3H), 1.78-1.70 (m, 1H), 1.71-1.63 (m, 1H), 1.62-1.55 (m, 1H), 1.52-1.44 (m, 1H).

### Example 130

### Synthesis of 3-((4-(5-chloro-1-((4-fluoropiperidin-4-yl)methyl)-1H-indol-7-yl)pyrrolo[2,1-f][1,2,4]triazin-6-yl)methyl)pyrimidine-2,4(1H,3H)-dione hydrochloride (130)

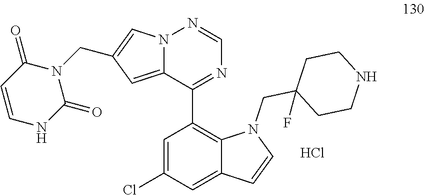 [SMILES: O=c1cc[nH]c(=O)n1Cc1cc2c(-c3cc(Cl)cc4ccn(CC5(F)CCNCC5)c34)ncnn2c1 ; Cl]

The title compound (130) was obtained as a hydrochloride salt in 11% overall yield in a similar way to Example 10 with the exception that, in the fourth step of the synthesis, the compound 89b was used instead of the compound 1b, and in the last step of the synthesis, the General Procedure IIIb was used instead of the General Procedure IIIa and the crude product was purified by preparative reversed-phase column chromatography (C-18, water+0.3‰ HCl (36%)/MeCN, 99:1 to 50:50, 30 min, 20 mL/min, Rₜ=22.10 min).

ESI-MS m/z for C₂₅H₂₄ClFN₇O₂ found 508.1/510.1 [M+H]⁺; Rₜ=0.98 min (Method A); ¹H NMR (700 MHz, Methanol-d₄) δ 8.60 (s, 1H), 8.16 (d, J=1.5 Hz, 1H), 7.86 (d, J=2.1 Hz, 1H), 7.47 (d, J=2.0 Hz, 1H), 7.42-7.37 (m, 2H), 6.85 (d, J=1.5 Hz, 1H), 6.73 (d, J=3.3 Hz, 1H), 5.71 (d, J=7.6 Hz, 1H), 5.20 (s, 2H), 4.38-4.31 (m, 2H), 3.36-3.32 (m, 1H), 3.18-3.12 (m, 2H), 2.91-2.84 (m, 2H), 1.69-1.57 (m, 2H), 1.47-1.41 (m, 2H).

### Example 131

### Synthesis of 3-((4-(5-chloro-1-((4-fluoropiperidin-4-yl)methyl)-1H-indol-7-yl)pyrrolo[2,1-f][1,2,4]triazin-6-yl)methyl)-1-(methyl-d₃)pyrimidine-2,4(1H,3H)-dione hydrochloride (131)

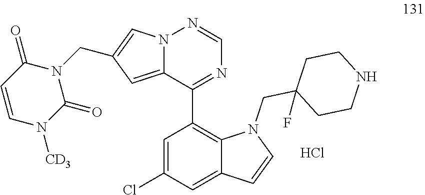 [SMILES: [2H]C([2H])([2H])n1ccc(=O)n(Cc2cc3c(-c4cc(Cl)cc5ccn(CC6(F)CCNCC6)c45)ncnn3c2)c1=O ; Cl]

The title compound (131) was obtained as a hydrochloride salt in 32% overall yield in a similar way to Example 89 with the exception that, in the third step of the synthesis, the compound 10c was used instead of the compound 2a, in the fourth step of the synthesis, iodomethane-d₃ was used instead of 2-iodopropane and in the last step of the synthesis, the crude product was purified by preparative reversed-phase column chromatography (C-18, water+0.3‰ HCl (36%)/MeCN, 99:1 to 40:60, 30 min, 20 mL/min, Rₜ=19.50 min).

ESI-MS m/z for C₂₆H₂₃D3ClFN₇O₂ found 525.1/527.1 [M+H]⁺; Rₜ=1.03 min (Method A); ¹H NMR (700 MHz, Methanol-d₄) δ 8.64 (s, 1H), 8.25 (d, J=1.5 Hz, 1H), 7.88 (d, J=2.1 Hz, 1H), 7.56 (d, J=7.8 Hz, 1H), 7.48 (d, J=2.1 Hz, 1H), 7.44-7.38 (m, 1H), 6.93 (d, J=1.5 Hz, 1H), 6.74 (d, J=3.2 Hz, 1H), 5.73 (d, J=7.8 Hz, 1H), 5.23 (s, 2H), 4.38-4.27 (m, 2H), 3.20-3.12 (m, 2H), 2.92-2.83 (m, 2H), 1.76-1.60 (m, 2H), 1.46-1.37 (m, 2H).

### Example 132

### Synthesis of 3-((4-(5-chloro-1-((4-fluoropiperidin-4-yl)methyl)-1H-indol-7-yl)-7-fluoropyrrolo[2,1-f][1,2,4]triazin-6-yl)methyl)-1-methylpyrimidine-2,4(1H,3H)-dione hydrochloride (132)

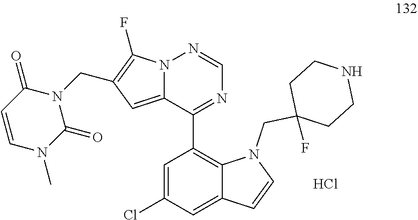 [SMILES: Cn1ccc(=O)n(Cc2cc3c(-c4cc(Cl)cc5ccn(CC6(F)CCNCC6)c45)ncnn3c2F)c1=O ; Cl]

### Step 1.

### Synthesis of 3-((4-chloro-7-fluoropyrrolo[2,1-f][1,2,4]triazin-6-yl)methyl)-1-methylpyrimidine-2,4(1H,3H)-dione (132a)

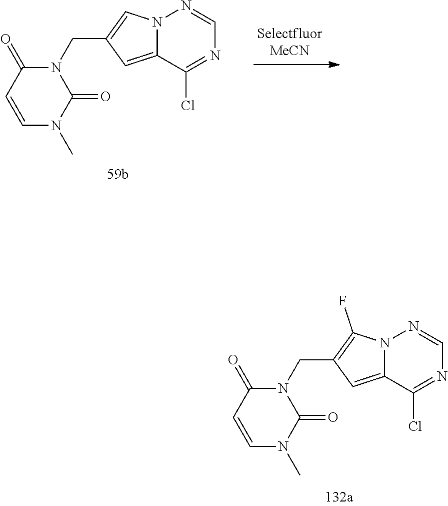 [SMILES: Cn1ccc(=O)n(Cc2cc3c(Cl)ncnn3c2)c1=O ; Cn1ccc(=O)n(Cc2cc3c(Cl)ncnn3c2F)c1=O]

To a solution of 59b (700 mg; 2.40 mmol) in anhydrous MeCN (48 mL) Selectfluor® fluorinating reagent 1-chloromethyl-4-fluoro-1,4-diazoniabicyclo[2.2.2]octane bis(tetrafluoroborate) (1063 mg; 0.622 mL, 2.88 mmol; 96%) was added and then the mixture was stirred at 80° C. overnight. The reaction progress was monitored by LC-MS. Then to this mixture another portion of Selectfluor® fluorinating reagent 1-chloromethyl-4-fluoro-1,4-diazoniabicyclo[2.2.2]octane bis(tetrafluoroborate) (531 mg; 0.311 mL; 1.44 mmol; 96%) was added and stirred at 80° C. for 2 days (the reaction was carried on in a Schlenk flask). When analysis indicated completion of the reaction, the solvent was removed in vacuo and the residue was purified by flash column chromatography on silica (DCM/MeOH, 100:0 to 0:100, v/v, 26 min, 30 mL/min) and then by preparative HPLC (Column: LUNA 5 μm SILICA [2] 100 A H₁₇-314538 Dimensions: 21.200001 mm×250 mm 5 μm; hexane/AcOEt, 100:0 to 0:100, v/v, 22 min, 21 mL/min). Compound 132a was obtained in 7% yield (50 mg; 0.16 mmol).

ESI-MS m/z for C₁₂H₁₀ClFN₅O₂ found 309.9/311.9 [M+H]⁺; Rₜ=0.91 min (Method A); ¹H NMR (700 MHz, CDCl₃) δ 8.19 (s, 1H), 7.17-7.11 (m, 1H), 7.00 (d, J=5.1 Hz, 1H), 5.78 (d, J=7.9 Hz, 1H), 5.24 (s, 2H), 3.41 (s, 3H).

### Step 2.

### Synthesis of tert-butyl 4-((5-chloro-7-(7-fluoro-6-((3-methyl-2,6-dioxo-3,6-dihydropyrimidin-1(2H)-yl)methyl)pyrrolo[2,1-f][1,2,4]triazin-4-yl)-1H-indol-1-yl)methyl)-4-fluoropiperidine-1-carboxylate (132b)

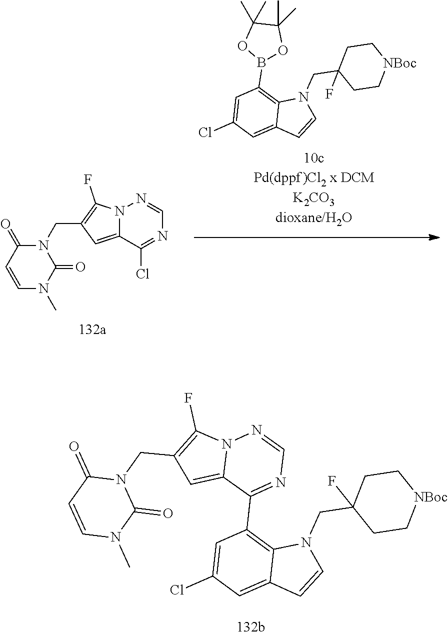 [SMILES: CC(C)(C)OC(=O)N1CCC(F)(Cn2ccc3cc(Cl)cc(B4OC(C)(C)C(C)(C)O4)c32)CC1 ; Cn1ccc(=O)n(Cc2cc3c(Cl)ncnn3c2F)c1=O ; Cn1ccc(=O)n(Cc2cc3c(-c4cc(Cl)cc5ccn(CC6(F)CCN(C(=O)OC(C)(C)C)CC6)c45)ncnn3c2F)c1=O]

The title compound (132b) was obtained from 132a (50 mg; 0.16 mmol) and from 10c (73 mg; 0.07 mmol; 50% and then: 142 mg; 0.09 mmol; 30%) according to the General Procedure IV in 87% yield (90 mg; 0.14 mmol). The crude product was purified by flash column chromatography on silica (hexane/AcOEt, 100:0 to 0:100, v/v, 15 min, 13 mL/min).

ESI-MS m/z for C₃₁H₃₃ClF₂N₇O₄ found 640.2/642.2 [M+H]⁺; Rₜ=1.73 min (Method A)

### Step 3.

### Synthesis of 3-((4-(5-chloro-1-((4-fluoropiperidin-4-yl)methyl)-1H-indol-7-yl)-7-fluoropyrrolo[2,1-f][1,2,4]triazin-6-yl)methyl)-1-methylpyrimidine-2,4(1H,3H)-dione hydrochloride (132)

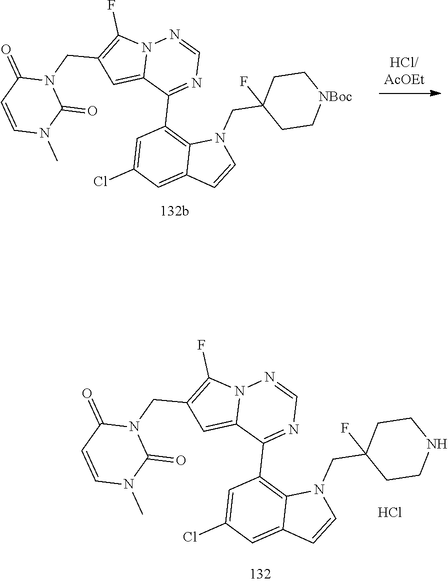 [SMILES: Cn1ccc(=O)n(Cc2cc3c(-c4cc(Cl)cc5ccn(CC6(F)CCN(C(=O)OC(C)(C)C)CC6)c45)ncnn3c2F)c1=O ; Cn1ccc(=O)n(Cc2cc3c(-c4cc(Cl)cc5ccn(CC6(F)CCNCC6)c45)ncnn3c2F)c1=O ; Cl]

The title compound (132) was obtained as a hydrochloride salt from 132b (90 mg; 0.14 mmol) according to the General Procedure IIIa in 14% yield (11 mg; 0.02 mmol). The crude product was purified by preparative reversed-phase column chromatography (C-18, water+0.30‰ HCl (36%)/MeCN, 99:1 to 30:70, 30 min, 20 mL/min, Rₜ=17.25 min).

ESI-MS m/z for C₂₆H₂₅ClF₂N₇O₂ found 540.4/542.4 [M+H]⁺; Rₜ=0.99 min (Method A); ¹H NMR (700 MHz, Methanol-d₄) δ 8.59 (s, 1H), 7.83 (d, J=2.1 Hz, 1H), 7.56 (d, J=7.8 Hz, 1H), 7.44 (d, J=2.1 Hz, 1H), 7.40-7.36 (m, 1H), 6.76 (d, J=5.1 Hz, 1H), 6.71 (d, J=3.2 Hz, 1H), 5.73 (d, J=7.8 Hz, 1H), 5.23 (s, 2H), 4.42 (d, J=22.9 Hz, 2H), 3.37 (s, 3H), 3.19-3.13 (m, 2H), 2.94-2.85 (m, 2H), 1.72-1.60 (m, 3H), 1.52-1.44 (m, 2H).

### Biological Assay

### Enzymatic Assay to Determine % Value of Inhibition of USP7 Activity at 1 μM of USP7

A 45 μl reaction volume containing full-length USP7 (0.5-1.0 nM) in 50 mM HEPES pH 7.5, 150 mM NaCl, 2 mM DTT, 1 mg/ml BSA and 0.05% Tween20 (DUB assay buffer) was assembled in wells of 96 well half area black flat bottom plates. All test compounds were first dissolved to 30 mM stocks in DMSO and subsequently introduced to enzyme 15 min after USP7 incubation with DTT at room temperature. The final concentration of tested compounds was 1 μM. The enzymatic reaction was started by adding 0.5 μM Ubiquitin-Rhodamine 110 (final concentration) and allowed to proceed for 30-60 min at 25° C., 250 RPM shaking before Rhodamine fluorescence (485 nm excitation/535 nm emission) was measured using Tecan Spark M10 plate reader. Percentage of inhibition at 1 μM was determined by the mean of two repetitions for these concentrations using Microsoft Excel software.

### Enzymatic Assay to Determine IC₅₀ of USP7 Inhibitors

The IC₅₀ is the concentration of an inhibitor where the measured enzyme activity is reduced by half. In the case of USP7 deubiquitinase, the IC₅₀ of an USP7 inhibitor is the molar concentration of the compound that inhibits 50% of the activity observed in a USP7-mediated-ubiquitin-rhodamine cleavage assay. For the inhibitors disclosed herein, their potency was measured using the following method.

A 45 μl reaction volume containing full-length USP7 (0.5-1.0 nM) in 50 mM HEPES pH 7.5, 150 mM NaCl, 2 mM DTT, 1 mg/ml BSA and 0.05% Tween20 was assembled in wells of 96 well half area black flat bottom plates. Test compounds were first dissolved to 30 mM stocks in DMSO and subsequently introduced to enzyme 15 min after USP7 incubation with DTT at room temperature. The enzymatic reaction was started by adding 0.5 μM Ubiquitin-Rhodamine 110 (final concentration) and allowed to proceed for 30-60 min at 25° C., 250 RPM shaking before Rhodamine fluorescence (485 nm excitation/535 nm emission) was measured using Tecan Spark M10 plate reader. The IC₅₀ values were determined by data fitting to variable slope model four-parameter dose-response curve using GraphPad 7.05.

The compounds disclosed in Table 1 below for which IC₅₀ values towards USP7 have been calculated as described above are characterized as falling into the following groups:
A: <0.5 μM;
B: 0.5-1 μM;
C: 1-10 μM;
D: >10 M.
If the IC₅₀ value has not been determined yet, a percent value of inhibition of USP7 activity at 1 μM of the test compound is specified. The following percentage ranges correspond to the corresponding letters with an asterisk; the compounds that lack IC₅₀ values are characterized as falling into the following groups:
A*: >70% inhibition;
B*: 50-70% inhibition;
C*: 20-49% inhibition;
D*: <20% inhibition.

TABLE 1

| | | USP7 IC₅₀ |
|---|---|---|
| Ex. No. | Structure | or % inhibition |
| 1. | 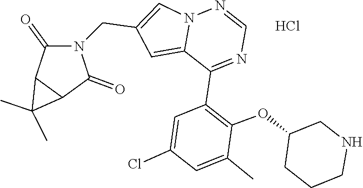 [SMILES: Cc1cc(Cl)cc(-c2ncnn3cc(CN4C(=O)C5C(C4=O)C5(C)C)cc23)c1O[C@H]1CCCNC1 ; Cl] | A |
| 2. | 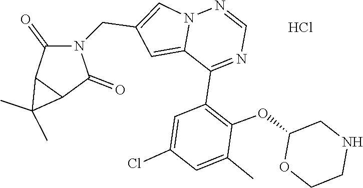 [SMILES: Cc1cc(Cl)cc(-c2ncnn3cc(CN4C(=O)C5C(C4=O)C5(C)C)cc23)c1O[C@@H]1CNCCO1 ; Cl] | A |
| 3. | 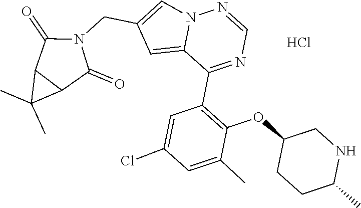 | C* |
| 4. | 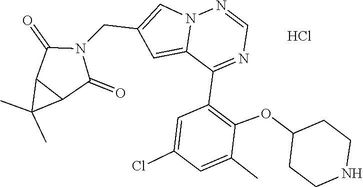 [SMILES: Cc1cc(Cl)cc(-c2ncnn3cc(CN4C(=O)C5C(C4=O)C5(C)C)cc23)c1OC1CCNCC1 ; Cl] | B* |
| 5. | 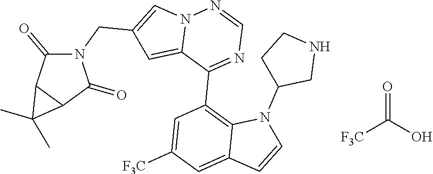 [SMILES: CC1(C)C2C(=O)N(Cc3cc4c(-c5cc(C(F)(F)F)cc6ccn(C7CCNC7)c56)ncnn4c3)C(=O)C21 ; O=C(O)C(F)(F)F] | C* |
| 6. | 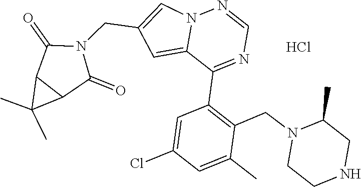 [SMILES: Cc1cc(Cl)cc(-c2ncnn3cc(CN4C(=O)C5C(C4=O)C5(C)C)cc23)c1CN1CCNC[C@@H]1C ; Cl] | A |
| 7. | 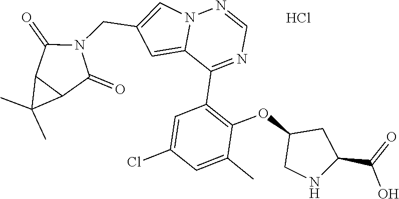 [SMILES: Cc1cc(Cl)cc(-c2ncnn3cc(CN4C(=O)C5C(C4=O)C5(C)C)cc23)c1O[C@@H]1CN[C@H](C(=O)O)C1 ; Cl] | D* |
| 8. | 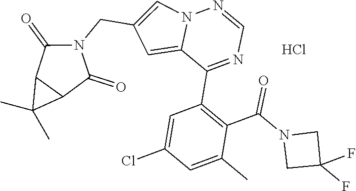 [SMILES: Cc1cc(Cl)cc(-c2ncnn3cc(CN4C(=O)C5C(C4=O)C5(C)C)cc23)c1C(=O)N1CC(F)(F)C1 ; Cl] | C* |
| 9. | 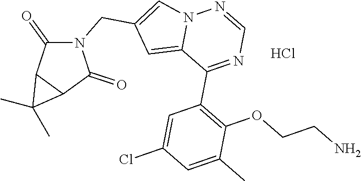 [SMILES: Cc1cc(Cl)cc(-c2ncnn3cc(CN4C(=O)C5C(C4=O)C5(C)C)cc23)c1OCCN ; Cl] | B* |
| 10. | 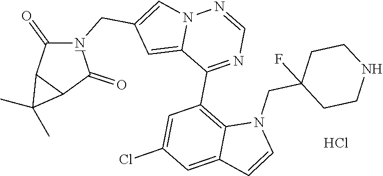 [SMILES: CC1(C)C2C(=O)N(Cc3cc4c(-c5cc(Cl)cc6ccn(CC7(F)CCNCC7)c56)ncnn4c3)C(=O)C21 ; Cl] | A |
| 11. | 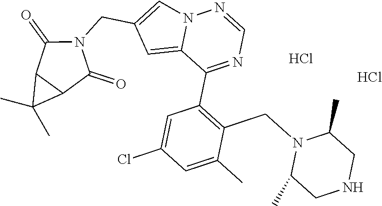 [SMILES: Cc1cc(Cl)cc(-c2ncnn3cc(CN4C(=O)C5C(C4=O)C5(C)C)cc23)c1CN1[C@@H](C)CNC[C@@H]1C ; Cl ; Cl] | A |
| 12. | 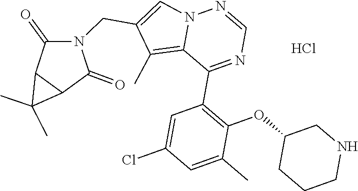 [SMILES: Cc1cc(Cl)cc(-c2ncnn3cc(CN4C(=O)C5C(C4=O)C5(C)C)c(C)c23)c1O[C@H]1CCCNC1 ; Cl] | B* |
| 13. | 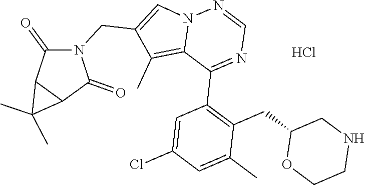 [SMILES: Cc1cc(Cl)cc(-c2ncnn3cc(CN4C(=O)C5C(C4=O)C5(C)C)c(C)c23)c1C[C@@H]1CNCCO1 ; Cl] | B |
| 14. | 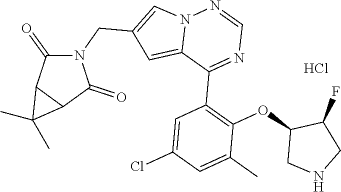 [SMILES: Cc1cc(Cl)cc(-c2ncnn3cc(CN4C(=O)C5C(C4=O)C5(C)C)cc23)c1O[C@@H]1CNC[C@@H]1F ; Cl] | A |
| 15. | 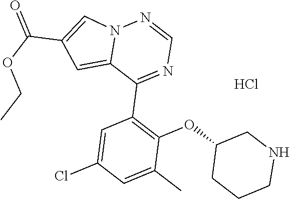 [SMILES: CCOC(=O)c1cc2c(-c3cc(Cl)cc(C)c3O[C@H]3CCCNC3)ncnn2c1 ; Cl] | C* |
| 16. | 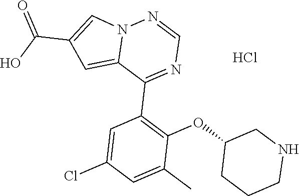 [SMILES: Cc1cc(Cl)cc(-c2ncnn3cc(C(=O)O)cc23)c1O[C@H]1CCCNC1 ; Cl] | D* |
| 17. | 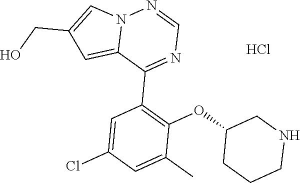 [SMILES: Cc1cc(Cl)cc(-c2ncnn3cc(CO)cc23)c1O[C@H]1CCCNC1 ; Cl] | D* |
| 18. | 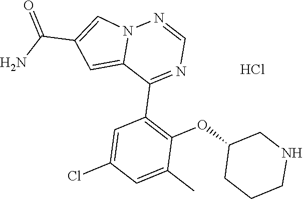 [SMILES: Cc1cc(Cl)cc(-c2ncnn3cc(C(N)=O)cc23)c1O[C@H]1CCCNC1 ; Cl] | D* |
| 19. | 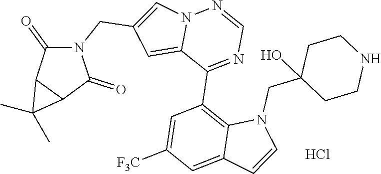 [SMILES: CC1(C)C2C(=O)N(Cc3cc4c(-c5cc(C(F)(F)F)cc6ccn(CC7(O)CCNCC7)c56)ncnn4c3)C(=O)C21 ; Cl] | B* |
| 20. | 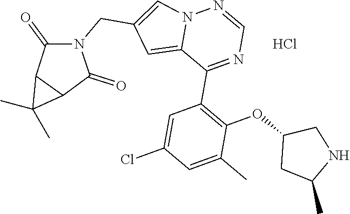 [SMILES: Cc1cc(Cl)cc(-c2ncnn3cc(CN4C(=O)C5C(C4=O)C5(C)C)cc23)c1O[C@@H]1CN[C@@H](C)C1 ; Cl] | C* |
| 21. | 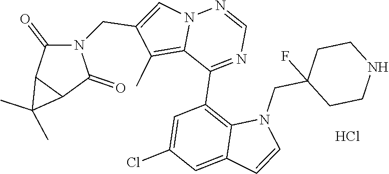 [SMILES: Cc1c(CN2C(=O)C3C(C2=O)C3(C)C)cn2ncnc(-c3cc(Cl)cc4ccn(CC5(F)CCNCC5)c34)c12 ; Cl] | B* |
| 22. | 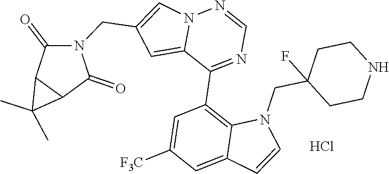 [SMILES: CC1(C)C2C(=O)N(Cc3cc4c(-c5cc(C(F)(F)F)cc6ccn(CC7(F)CCNCC7)c56)ncnn4c3)C(=O)C21 ; Cl] | A |
| 23. | 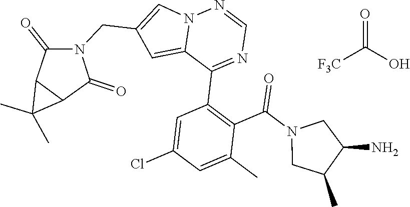 [SMILES: Cc1cc(Cl)cc(-c2ncnn3cc(CN4C(=O)C5C(C4=O)C5(C)C)cc23)c1C(=O)N1C[C@@H](N)[C@@H](C)C1 ; O=C(O)C(F)(F)F] | B* |
| 24. | 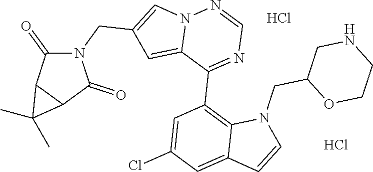 [SMILES: CC1(C)C2C(=O)N(Cc3cc4c(-c5cc(Cl)cc6ccn(CC7CNCCO7)c56)ncnn4c3)C(=O)C21 ; Cl ; Cl] | A |
| 25. | 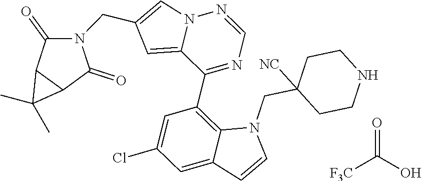 | A |
| 26. | 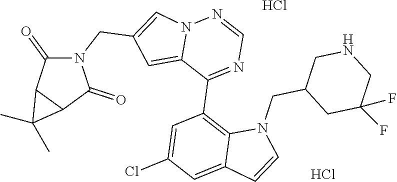 [SMILES: Cl ; CC1(C)C2C(=O)N(Cc3cc4c(-c5cc(Cl)cc6ccn(CC7CNCC(F)(F)C7)c56)ncnn4c3)C(=O)C21 ; Cl] | C* |
| 27. | 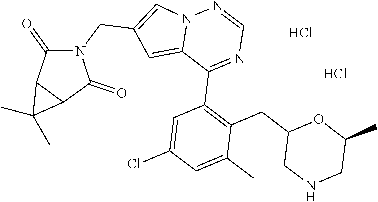 | A |
| 28. | 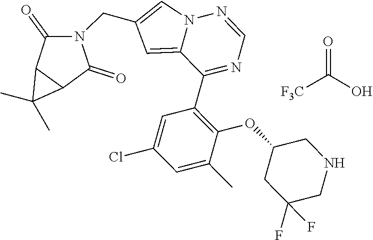 [SMILES: Cc1cc(Cl)cc(-c2ncnn3cc(CN4C(=O)C5C(C4=O)C5(C)C)cc23)c1O[C@@H]1CNCC(F)(F)C1 ; O=C(O)C(F)(F)F] | D* |
| 29. | 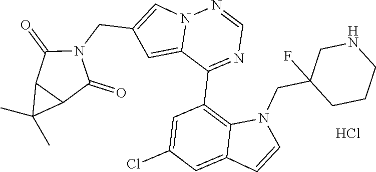 [SMILES: CC1(C)C2C(=O)N(Cc3cc4c(-c5cc(Cl)cc6ccn(CC7(F)CCCNC7)c56)ncnn4c3)C(=O)C21 ; Cl] | A |
| 30. | 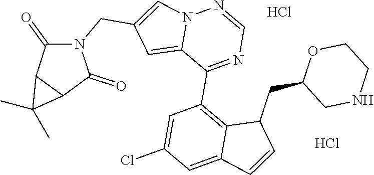 [SMILES: CC1(C)C2C(=O)N(Cc3cc4c(-c5cc(Cl)cc6c5C(C[C@@H]5CNCCO5)C=C6)ncnn4c3)C(=O)C21 ; Cl ; Cl] | A |
| 31. | 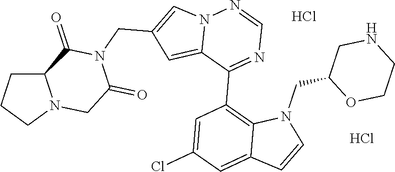 [SMILES: O=C1CN2CCC[C@H]2C(=O)N1Cc1cc2c(-c3cc(Cl)cc4ccn(C[C@@H]5CNCCO5)c34)ncnn2c1 ; Cl ; Cl] | B* |
| 32. | 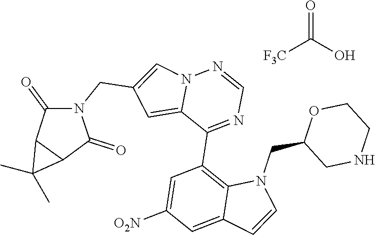 [SMILES: O=C(O)C(F)(F)F ; CC1(C)C2C(=O)N(Cc3cc4c(-c5cc([N+](=O)[O-])cc6ccn(C[C@@H]7CNCCO7)c56)ncnn4c3)C(=O)C21] | A |
| 33. | 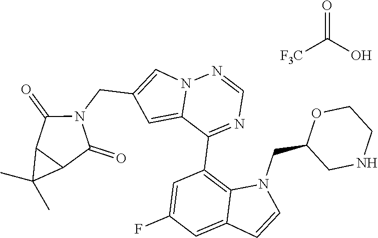 [SMILES: O=C(O)C(F)(F)F ; CC1(C)C2C(=O)N(Cc3cc4c(-c5cc(F)cc6ccn(C[C@@H]7CNCCO7)c56)ncnn4c3)C(=O)C21] | A* |
| 34. | 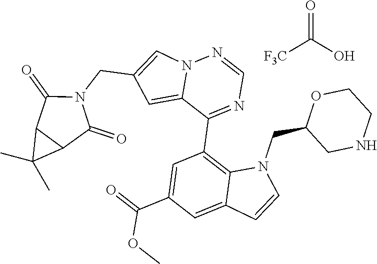 | D* |
| 35. | 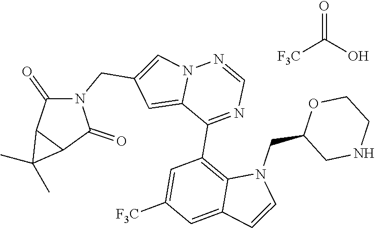 | A |
| 36. | 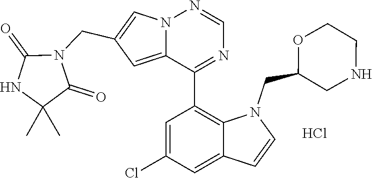 [SMILES: CC1(C)NC(=O)N(Cc2cc3c(-c4cc(Cl)cc5ccn(C[C@@H]6CNCCO6)c45)ncnn3c2)C1=O ; Cl] | B* |
| 37. | 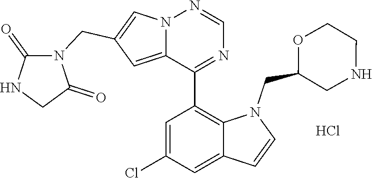 [SMILES: O=C1CNC(=O)N1Cc1cc2c(-c3cc(Cl)cc4ccn(C[C@@H]5CNCCO5)c34)ncnn2c1 ; Cl] | A |
| 38. | 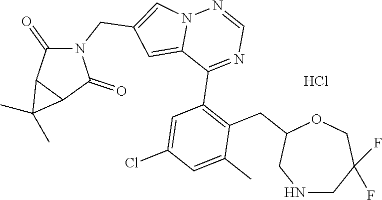 [SMILES: Cc1cc(Cl)cc(-c2ncnn3cc(CN4C(=O)C5C(C4=O)C5(C)C)cc23)c1CC1CNCC(F)(F)CO1 ; Cl] | D* |
| 39. | 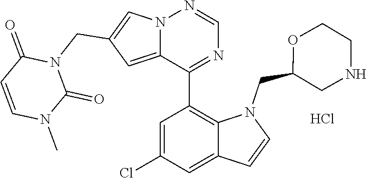 [SMILES: Cn1ccc(=O)n(Cc2cc3c(-c4cc(Cl)cc5ccn(C[C@@H]6CNCCO6)c45)ncnn3c2)c1=O ; Cl] | A |
| 40. | 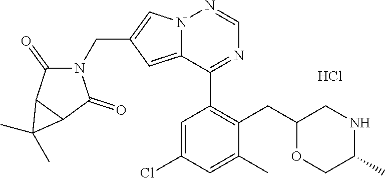 | B* |
| 41. | 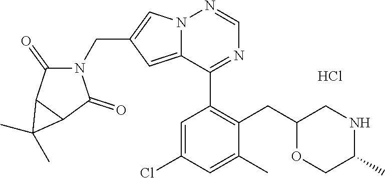 | A |
| 42. | 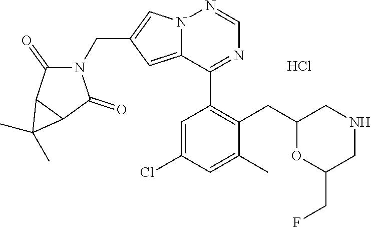 [SMILES: Cc1cc(Cl)cc(-c2ncnn3cc(CN4C(=O)C5C(C4=O)C5(C)C)cc23)c1CC1CNCC(CF)O1 ; Cl] | B* |
| 43. | 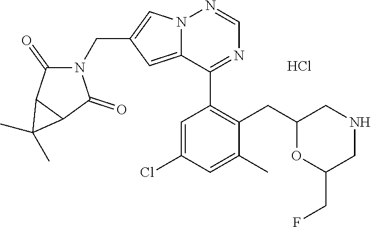 [SMILES: Cc1cc(Cl)cc(-c2ncnn3cc(CN4C(=O)C5C(C4=O)C5(C)C)cc23)c1CC1CNCC(CF)O1 ; Cl] | C* |
| 44. | 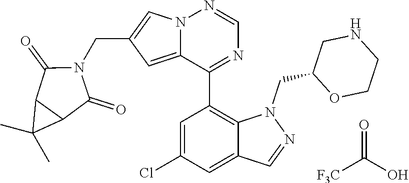 | A |
| 45. | 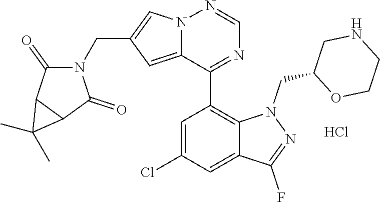 [SMILES: CC1(C)C2C(=O)N(Cc3cc4c(-c5cc(Cl)cc6c(F)nn(C[C@@H]7CNCCO7)c56)ncnn4c3)C(=O)C21 ; Cl] | A |
| 46. | 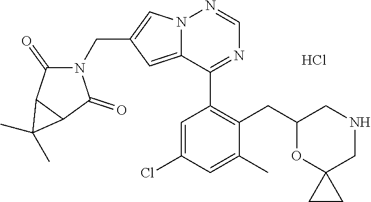 [SMILES: Cc1cc(Cl)cc(-c2ncnn3cc(CN4C(=O)C5C(C4=O)C5(C)C)cc23)c1CC1CNCC2(CC2)O1 ; Cl] | A |
| 47. | 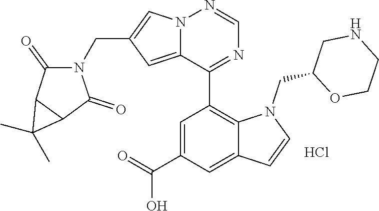 [SMILES: CC1(C)C2C(=O)N(Cc3cc4c(-c5cc(C(=O)O)cc6ccn(C[C@@H]7CNCCO7)c56)ncnn4c3)C(=O)C21 ; Cl] | D* |
| 48. | 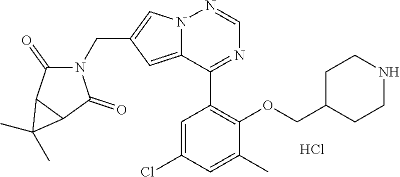 [SMILES: Cc1cc(Cl)cc(-c2ncnn3cc(CN4C(=O)C5C(C4=O)C5(C)C)cc23)c1OCC1CCNCC1 ; Cl] | A |
| 49. | 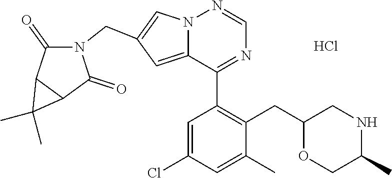 | A |
| 50. | 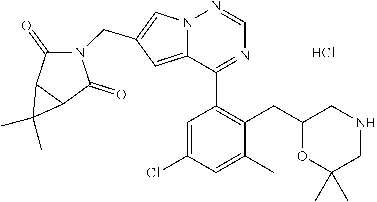 [SMILES: Cc1cc(Cl)cc(-c2ncnn3cc(CN4C(=O)C5C(C4=O)C5(C)C)cc23)c1CC1CNCC(C)(C)O1 ; Cl] | A |
| 51. | 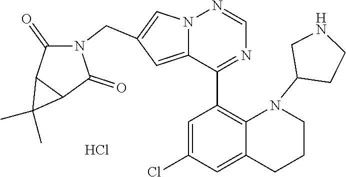 [SMILES: CC1(C)C2C(=O)N(Cc3cc4c(-c5cc(Cl)cc6c5N(C5CCNC5)CCC6)ncnn4c3)C(=O)C21 ; Cl] | A |
| 52. | 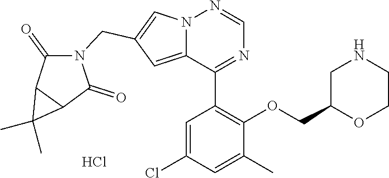 [SMILES: Cc1cc(Cl)cc(-c2ncnn3cc(CN4C(=O)C5C(C4=O)C5(C)C)cc23)c1OC[C@H]1CNCCO1 ; Cl] | C* |
| 53. | 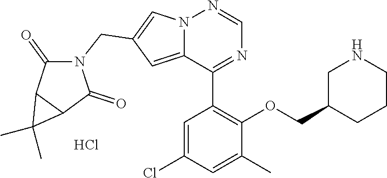 [SMILES: Cc1cc(Cl)cc(-c2ncnn3cc(CN4C(=O)C5C(C4=O)C5(C)C)cc23)c1OC[C@@H]1CCCNC1 ; Cl] | C* |
| 54. | 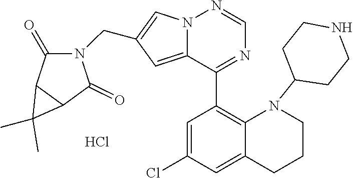 [SMILES: CC1(C)C2C(=O)N(Cc3cc4c(-c5cc(Cl)cc6c5N(C5CCNCC5)CCC6)ncnn4c3)C(=O)C21 ; Cl] | A* |
| 55. | 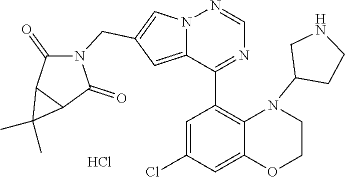 [SMILES: CC1(C)C2C(=O)N(Cc3cc4c(-c5cc(Cl)cc6c5N(C5CCNC5)CCO6)ncnn4c3)C(=O)C21 ; Cl] | A |
| 56. | 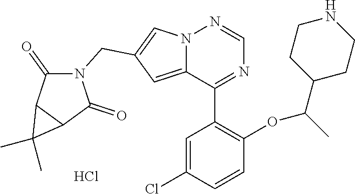 [SMILES: CC(Oc1ccc(Cl)cc1-c1ncnn2cc(CN3C(=O)C4C(C3=O)C4(C)C)cc12)C1CCNCC1 ; Cl] | B* |
| 57. | 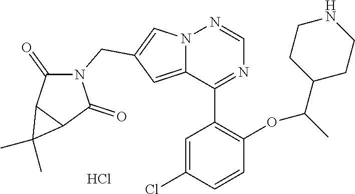 [SMILES: CC(Oc1ccc(Cl)cc1-c1ncnn2cc(CN3C(=O)C4C(C3=O)C4(C)C)cc12)C1CCNCC1 ; Cl] | C* |
| 58. | 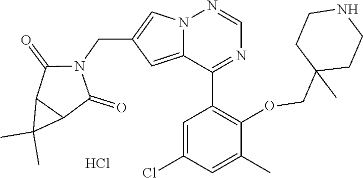 [SMILES: Cc1cc(Cl)cc(-c2ncnn3cc(CN4C(=O)C5C(C4=O)C5(C)C)cc23)c1OCC1(C)CCNCC1 ; Cl] | A |
| 59. | 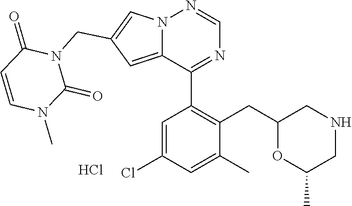 [SMILES: Cc1cc(Cl)cc(-c2ncnn3cc(Cn4c(=O)ccn(C)c4=O)cc23)c1CC1CNC[C@H](C)O1 ; Cl] | A |
| 60. | 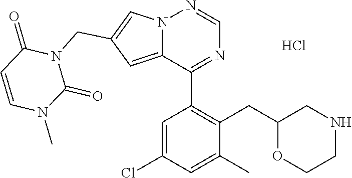 [SMILES: Cc1cc(Cl)cc(-c2ncnn3cc(Cn4c(=O)ccn(C)c4=O)cc23)c1CC1CNCCO1 ; Cl] | A |
| 61. | 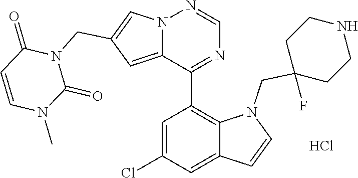 [SMILES: Cn1ccc(=O)n(Cc2cc3c(-c4cc(Cl)cc5ccn(CC6(F)CCNCC6)c45)ncnn3c2)c1=O ; Cl] | A |
| 62. | 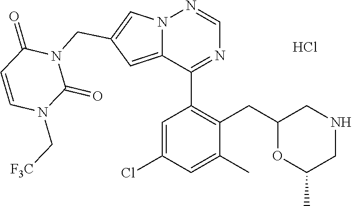 [SMILES: Cc1cc(Cl)cc(-c2ncnn3cc(Cn4c(=O)ccn(CC(F)(F)F)c4=O)cc23)c1CC1CNC[C@H](C)O1 ; Cl] | A |
| 63. | 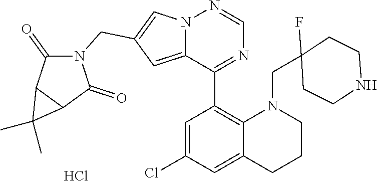 [SMILES: CC1(C)C2C(=O)N(Cc3cc4c(-c5cc(Cl)cc6c5N(CC5(F)CCNCC5)CCC6)ncnn4c3)C(=O)C21 ; Cl] | A |
| 64. | 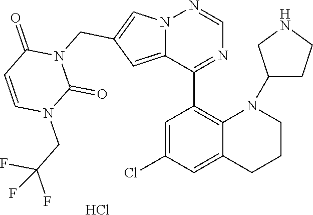 [SMILES: O=c1ccn(CC(F)(F)F)c(=O)n1Cc1cc2c(-c3cc(Cl)cc4c3N(C3CCNC3)CCC4)ncnn2c1 ; Cl] | A* |
| 65. | 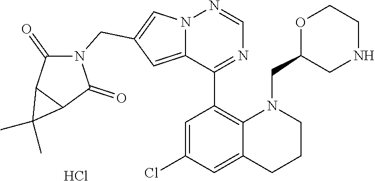 [SMILES: CC1(C)C2C(=O)N(Cc3cc4c(-c5cc(Cl)cc6c5N(C[C@@H]5CNCCO5)CCC6)ncnn4c3)C(=O)C21 ; Cl] | C* |
| 66. | 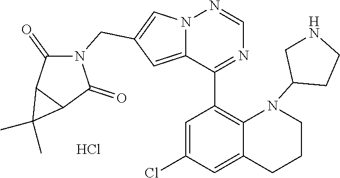 [SMILES: CC1(C)C2C(=O)N(Cc3cc4c(-c5cc(Cl)cc6c5N(C5CCNC5)CCC6)ncnn4c3)C(=O)C21 ; Cl] | A |
| 67. | 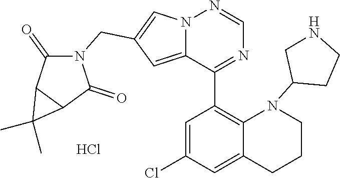 [SMILES: CC1(C)C2C(=O)N(Cc3cc4c(-c5cc(Cl)cc6c5N(C5CCNC5)CCC6)ncnn4c3)C(=O)C21 ; Cl] | A |
| 68. | 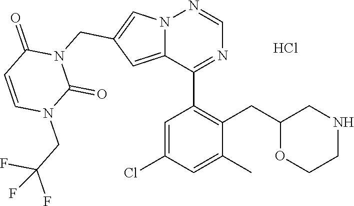 [SMILES: Cc1cc(Cl)cc(-c2ncnn3cc(Cn4c(=O)ccn(CC(F)(F)F)c4=O)cc23)c1CC1CNCCO1 ; Cl] | A |
| 69. | 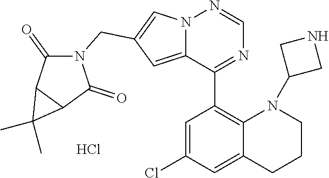 [SMILES: CC1(C)C2C(=O)N(Cc3cc4c(-c5cc(Cl)cc6c5N(C5CNC5)CCC6)ncnn4c3)C(=O)C21 ; Cl] | A* |
| 70. | 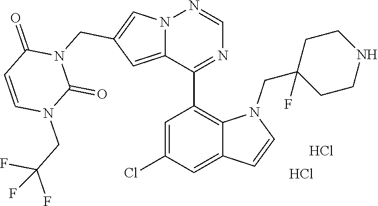 [SMILES: O=c1ccn(CC(F)(F)F)c(=O)n1Cc1cc2c(-c3cc(Cl)cc4ccn(CC5(F)CCNCC5)c34)ncnn2c1 ; Cl ; Cl] | A |
| 71. | 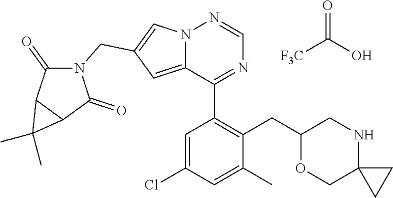 [SMILES: Cc1cc(Cl)cc(-c2ncnn3cc(CN4C(=O)C5C(C4=O)C5(C)C)cc23)c1CC1CNC2(CC2)CO1 ; O=C(O)C(F)(F)F] | C* |
| 72. | 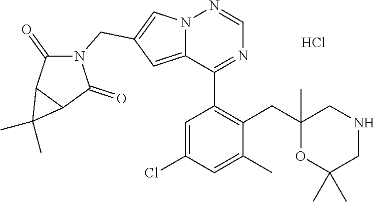 [SMILES: Cc1cc(Cl)cc(-c2ncnn3cc(CN4C(=O)C5C(C4=O)C5(C)C)cc23)c1CC1(C)CNCC(C)(C)O1 ; Cl] | A* |
| 73. | 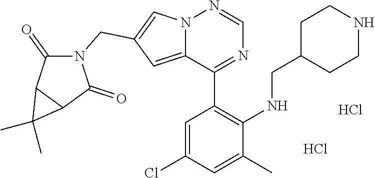 [SMILES: Cc1cc(Cl)cc(-c2ncnn3cc(CN4C(=O)C5C(C4=O)C5(C)C)cc23)c1NCC1CCNCC1 ; Cl ; Cl] | A* |
| 74. | 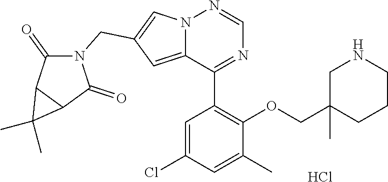 [SMILES: Cc1cc(Cl)cc(-c2ncnn3cc(CN4C(=O)C5C(C4=O)C5(C)C)cc23)c1OCC1(C)CCCNC1 ; Cl] | B* |
| 75. | 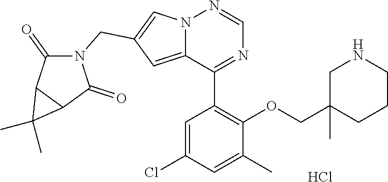 [SMILES: Cc1cc(Cl)cc(-c2ncnn3cc(CN4C(=O)C5C(C4=O)C5(C)C)cc23)c1OCC1(C)CCCNC1 ; Cl] | B* |
| 76. | 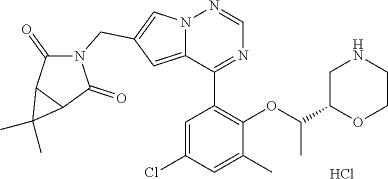 [SMILES: Cc1cc(Cl)cc(-c2ncnn3cc(CN4C(=O)C5C(C4=O)C5(C)C)cc23)c1OC(C)[C@@H]1CNCCO1 ; Cl] | B* |
| 77. | 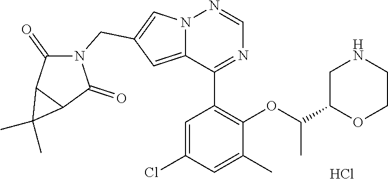 [SMILES: Cc1cc(Cl)cc(-c2ncnn3cc(CN4C(=O)C5C(C4=O)C5(C)C)cc23)c1OC(C)[C@@H]1CNCCO1 ; Cl] | D* |
| 78. | 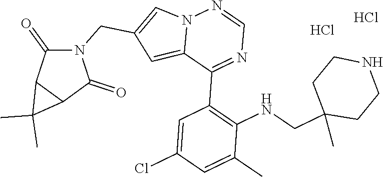 [SMILES: Cc1cc(Cl)cc(-c2ncnn3cc(CN4C(=O)C5C(C4=O)C5(C)C)cc23)c1NCC1(C)CCNCC1 ; Cl ; Cl] | A |
| 79. | 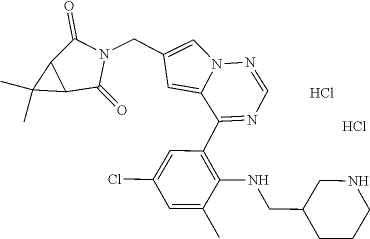 [SMILES: Cc1cc(Cl)cc(-c2ncnn3cc(CN4C(=O)C5C(C4=O)C5(C)C)cc23)c1NCC1CCCNC1 ; Cl ; Cl] | B* |
| 80. | 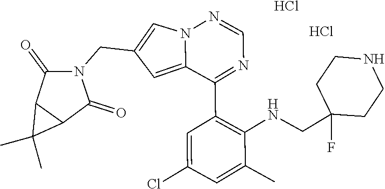 [SMILES: Cl ; Cc1cc(Cl)cc(-c2ncnn3cc(CN4C(=O)C5C(C4=O)C5(C)C)cc23)c1NCC1(F)CCNCC1 ; Cl] | A |
| 81. | 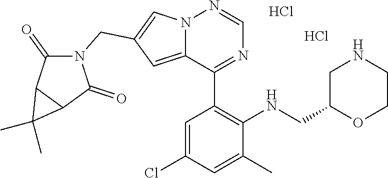 [SMILES: Cc1cc(Cl)cc(-c2ncnn3cc(CN4C(=O)C5C(C4=O)C5(C)C)cc23)c1NC[C@@H]1CNCCO1 ; Cl ; Cl] | C* |
| 82. | 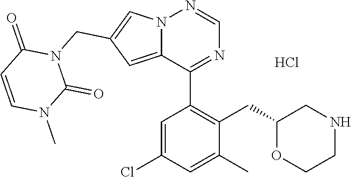 [SMILES: Cc1cc(Cl)cc(-c2ncnn3cc(Cn4c(=O)ccn(C)c4=O)cc23)c1C[C@@H]1CNCCO1 ; Cl] | A |
| 83. | 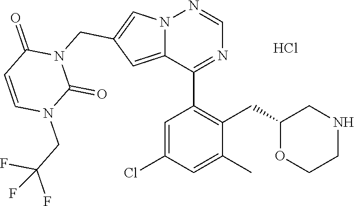 [SMILES: Cc1cc(Cl)cc(-c2ncnn3cc(Cn4c(=O)ccn(CC(F)(F)F)c4=O)cc23)c1C[C@@H]1CNCCO1 ; Cl] | A |
| 84. | 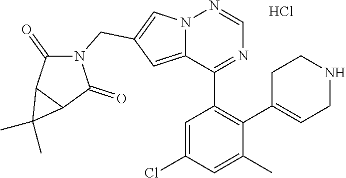 [SMILES: Cc1cc(Cl)cc(-c2ncnn3cc(CN4C(=O)C5C(C4=O)C5(C)C)cc23)c1C1=CCNCC1 ; Cl] | D* |
| 85. | 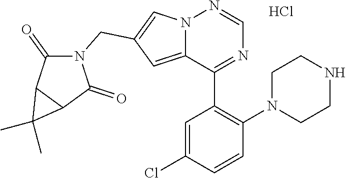 [SMILES: CC1(C)C2C(=O)N(Cc3cc4c(-c5cc(Cl)ccc5N5CCNCC5)ncnn4c3)C(=O)C21 ; Cl] | D* |
| 86. | 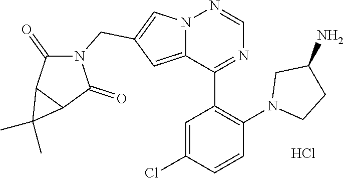 [SMILES: CC1(C)C2C(=O)N(Cc3cc4c(-c5cc(Cl)ccc5N5CC[C@H](N)C5)ncnn4c3)C(=O)C21 ; Cl] | C* |
| 87. | 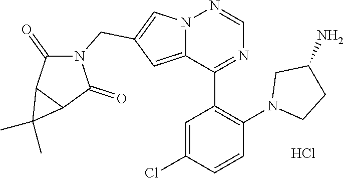 [SMILES: CC1(C)C2C(=O)N(Cc3cc4c(-c5cc(Cl)ccc5N5CC[C@@H](N)C5)ncnn4c3)C(=O)C21 ; Cl] | C* |
| 88. | 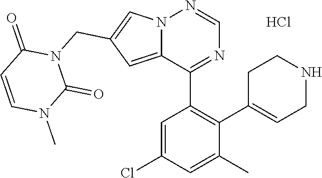 [SMILES: Cc1cc(Cl)cc(-c2ncnn3cc(Cn4c(=O)ccn(C)c4=O)cc23)c1C1=CCNCC1 ; Cl] | D* |
| 89. | 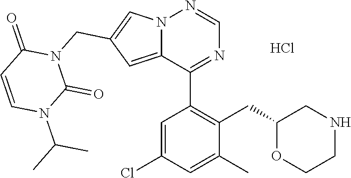 [SMILES: Cc1cc(Cl)cc(-c2ncnn3cc(Cn4c(=O)ccn(C(C)C)c4=O)cc23)c1C[C@@H]1CNCCO1 ; Cl] | A |
| 90. | 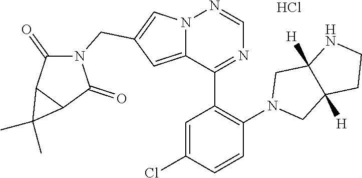 [SMILES: CC1(C)C2C(=O)N(Cc3cc4c(-c5cc(Cl)ccc5N5C[C@H]6CCN[C@H]6C5)ncnn4c3)C(=O)C21 ; Cl] | B* |
| 91. | 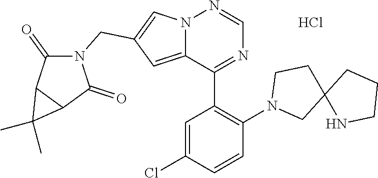 [SMILES: CC1(C)C2C(=O)N(Cc3cc4c(-c5cc(Cl)ccc5N5CCC6(CCCN6)C5)ncnn4c3)C(=O)C21 ; Cl] | C* |
| 92. | 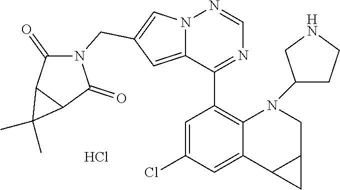 [SMILES: CC1(C)C2C(=O)N(Cc3cc4c(-c5cc(Cl)cc6c5N(C5CCNC5)CC5CC65)ncnn4c3)C(=O)C21 ; Cl] | B* |
| 93. | 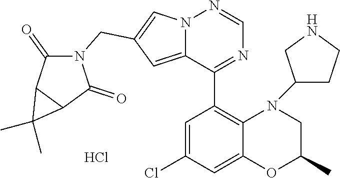 [SMILES: C[C@@H]1CN(C2CCNC2)c2c(cc(Cl)cc2-c2ncnn3cc(CN4C(=O)C5C(C4=O)C5(C)C)cc23)O1 ; Cl] | A |
| 94. | 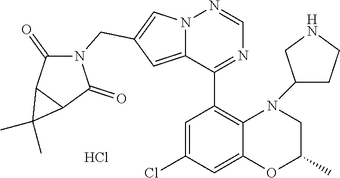 [SMILES: C[C@H]1CN(C2CCNC2)c2c(cc(Cl)cc2-c2ncnn3cc(CN4C(=O)C5C(C4=O)C5(C)C)cc23)O1 ; Cl] | A |
| 95. | 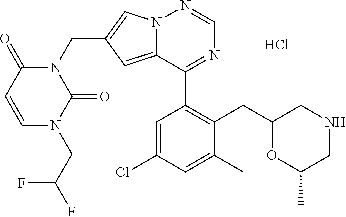 [SMILES: Cc1cc(Cl)cc(-c2ncnn3cc(Cn4c(=O)ccn(CC(F)F)c4=O)cc23)c1CC1CNC[C@H](C)O1 ; Cl] | A |
| 96. | 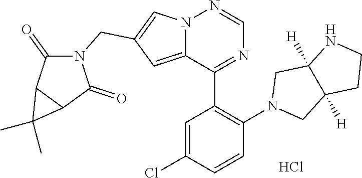 [SMILES: CC1(C)C2C(=O)N(Cc3cc4c(-c5cc(Cl)ccc5N5C[C@@H]6CCN[C@@H]6C5)ncnn4c3)C(=O)C21 ; Cl] | D* |
| 97. | 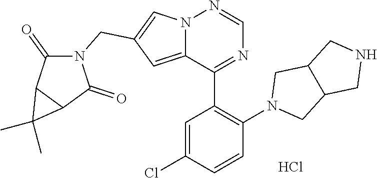 [SMILES: CC1(C)C2C(=O)N(Cc3cc4c(-c5cc(Cl)ccc5N5CC6CNCC6C5)ncnn4c3)C(=O)C21 ; Cl] | D* |
| 98. | 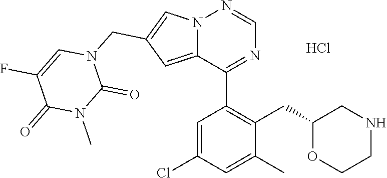 [SMILES: Cc1cc(Cl)cc(-c2ncnn3cc(Cn4cc(F)c(=O)n(C)c4=O)cc23)c1C[C@@H]1CNCCO1 ; Cl] | D* |
| 99. | 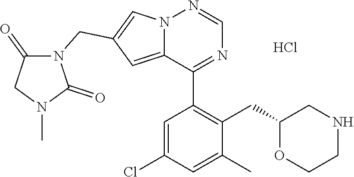 [SMILES: Cc1cc(Cl)cc(-c2ncnn3cc(CN4C(=O)CN(C)C4=O)cc23)c1C[C@@H]1CNCCO1 ; Cl] | A* |
| 100. | 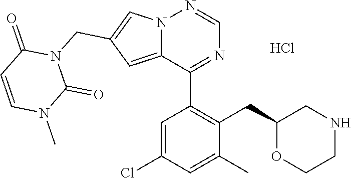 [SMILES: Cc1cc(Cl)cc(-c2ncnn3cc(Cn4c(=O)ccn(C)c4=O)cc23)c1C[C@H]1CNCCO1 ; Cl] | C* |
| 101. | 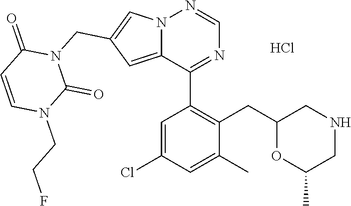 [SMILES: Cc1cc(Cl)cc(-c2ncnn3cc(Cn4c(=O)ccn(CCF)c4=O)cc23)c1CC1CNC[C@H](C)O1 ; Cl] | A |
| 102. | 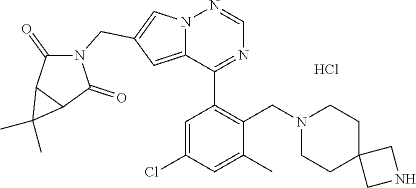 [SMILES: Cc1cc(Cl)cc(-c2ncnn3cc(CN4C(=O)C5C(C4=O)C5(C)C)cc23)c1CN1CCC2(CC1)CNC2 ; Cl] | C* |
| 103. | 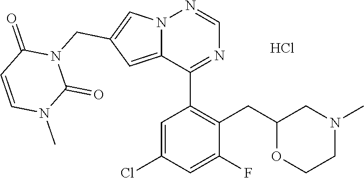 [SMILES: CN1CCOC(Cc2c(F)cc(Cl)cc2-c2ncnn3cc(Cn4c(=O)ccn(C)c4=O)cc23)C1 ; Cl] | C* |
| 104. | 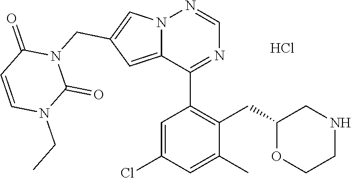 [SMILES: CCn1ccc(=O)n(Cc2cc3c(-c4cc(Cl)cc(C)c4C[C@@H]4CNCCO4)ncnn3c2)c1=O ; Cl] | A |
| 105. | 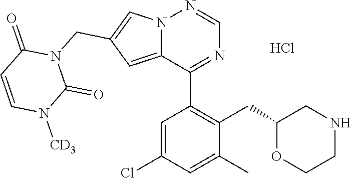 [SMILES: [2H]C([2H])([2H])n1ccc(=O)n(Cc2cc3c(-c4cc(Cl)cc(C)c4C[C@@H]4CNCCO4)ncnn3c2)c1=O ; Cl] | A |
| 106. | 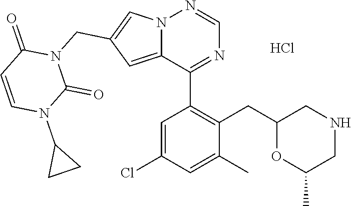 [SMILES: Cc1cc(Cl)cc(-c2ncnn3cc(Cn4c(=O)ccn(C5CC5)c4=O)cc23)c1CC1CNC[C@H](C)O1 ; Cl] | A |
| 107. | 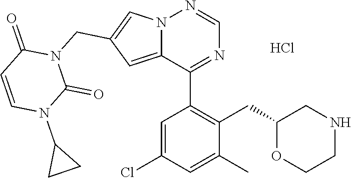 [SMILES: Cc1cc(Cl)cc(-c2ncnn3cc(Cn4c(=O)ccn(C5CC5)c4=O)cc23)c1C[C@@H]1CNCCO1 ; Cl] | A |
| 108. | 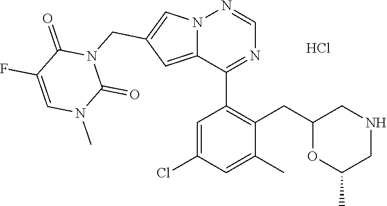 [SMILES: Cc1cc(Cl)cc(-c2ncnn3cc(Cn4c(=O)c(F)cn(C)c4=O)cc23)c1CC1CNC[C@H](C)O1 ; Cl] | A |
| 109. | 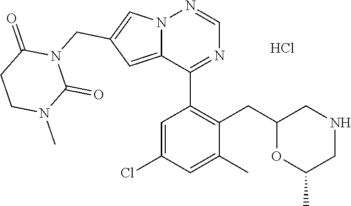 [SMILES: Cc1cc(Cl)cc(-c2ncnn3cc(CN4C(=O)CCN(C)C4=O)cc23)c1CC1CNC[C@H](C)O1 ; Cl] | A |
| 110. | 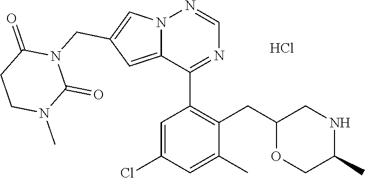 [SMILES: Cc1cc(Cl)cc(-c2ncnn3cc(CN4C(=O)CCN(C)C4=O)cc23)c1CC1CN[C@@H](C)CO1 ; Cl] | A |
| 111. | 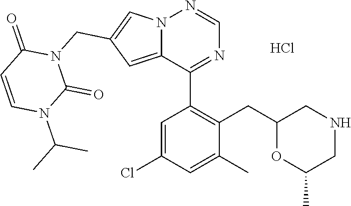 [SMILES: Cc1cc(Cl)cc(-c2ncnn3cc(Cn4c(=O)ccn(C(C)C)c4=O)cc23)c1CC1CNC[C@H](C)O1 ; Cl] | A |
| 112. | 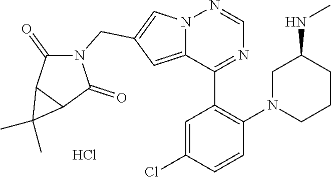 | B* |
| 113. | 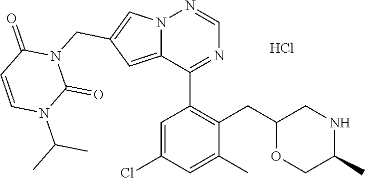 [SMILES: Cc1cc(Cl)cc(-c2ncnn3cc(Cn4c(=O)ccn(C(C)C)c4=O)cc23)c1CC1CN[C@@H](C)CO1 ; Cl] | A |
| 114. | 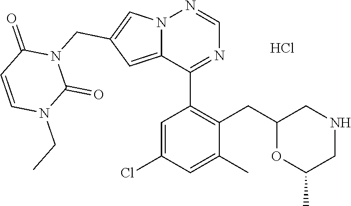 [SMILES: CCn1ccc(=O)n(Cc2cc3c(-c4cc(Cl)cc(C)c4CC4CNC[C@H](C)O4)ncnn3c2)c1=O ; Cl] | A |
| 115. | 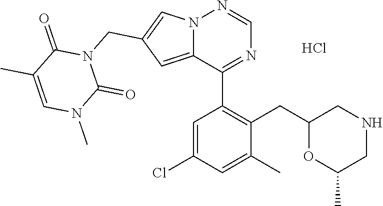 [SMILES: Cc1cc(Cl)cc(-c2ncnn3cc(Cn4c(=O)c(C)cn(C)c4=O)cc23)c1CC1CNC[C@H](C)O1 ; Cl] | A |
| 116. | 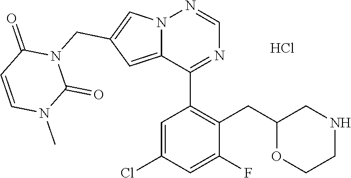 [SMILES: Cn1ccc(=O)n(Cc2cc3c(-c4cc(Cl)cc(F)c4CC4CNCCO4)ncnn3c2)c1=O ; Cl] | B* |
| 117. | 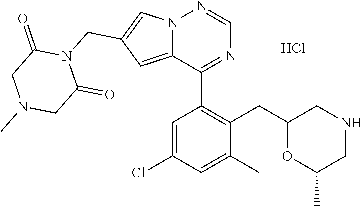 [SMILES: Cc1cc(Cl)cc(-c2ncnn3cc(CN4C(=O)CN(C)CC4=O)cc23)c1CC1CNC[C@H](C)O1 ; Cl] | A |
| 118. | 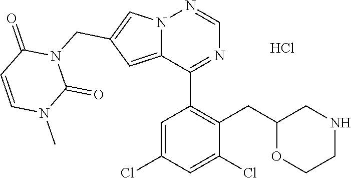 [SMILES: Cn1ccc(=O)n(Cc2cc3c(-c4cc(Cl)cc(Cl)c4CC4CNCCO4)ncnn3c2)c1=O ; Cl] | A |
| 119. | 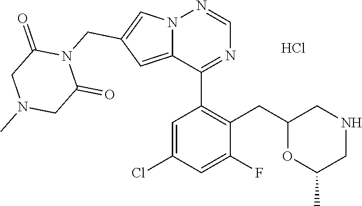 [SMILES: C[C@H]1CNCC(Cc2c(F)cc(Cl)cc2-c2ncnn3cc(CN4C(=O)CN(C)CC4=O)cc23)O1 ; Cl] | C* |
| 120. | 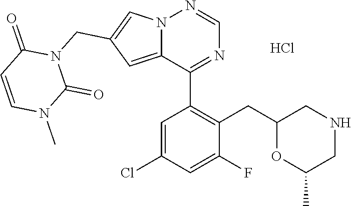 [SMILES: C[C@H]1CNCC(Cc2c(F)cc(Cl)cc2-c2ncnn3cc(Cn4c(=O)ccn(C)c4=O)cc23)O1 ; Cl] | A* |
| 121. | 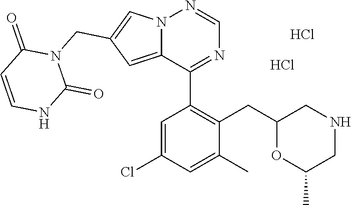 [SMILES: Cc1cc(Cl)cc(-c2ncnn3cc(Cn4c(=O)cc[nH]c4=O)cc23)c1CC1CNC[C@H](C)O1 ; Cl ; Cl] | B* |
| 122. | 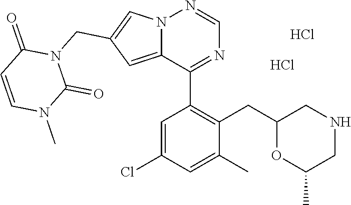 [SMILES: Cc1cc(Cl)cc(-c2ncnn3cc(Cn4c(=O)ccn(C)c4=O)cc23)c1CC1CNC[C@H](C)O1 ; Cl ; Cl] | C* |
| 123. | 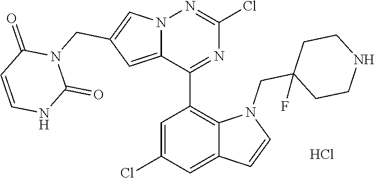 [SMILES: O=c1cc[nH]c(=O)n1Cc1cc2c(-c3cc(Cl)cc4ccn(CC5(F)CCNCC5)c34)nc(Cl)nn2c1 ; Cl] | D* |
| 124. | 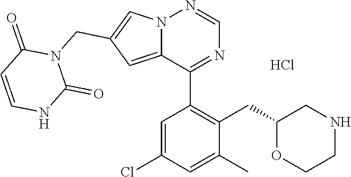 [SMILES: Cc1cc(Cl)cc(-c2ncnn3cc(Cn4c(=O)cc[nH]c4=O)cc23)c1C[C@@H]1CNCCO1 ; Cl] | A |
| 125. | 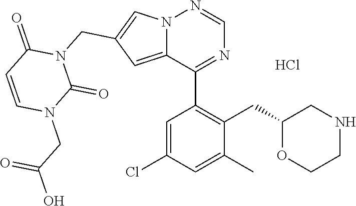 [SMILES: Cc1cc(Cl)cc(-c2ncnn3cc(Cn4c(=O)ccn(CC(=O)O)c4=O)cc23)c1C[C@@H]1CNCCO1 ; Cl] | C* |
| 126. | 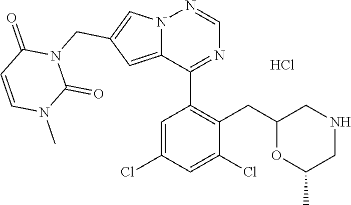 [SMILES: C[C@H]1CNCC(Cc2c(Cl)cc(Cl)cc2-c2ncnn3cc(Cn4c(=O)ccn(C)c4=O)cc23)O1 ; Cl] | A |
| 127. | 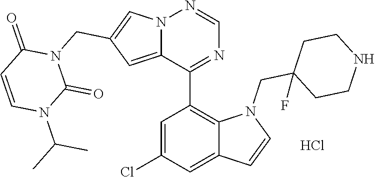 [SMILES: CC(C)n1ccc(=O)n(Cc2cc3c(-c4cc(Cl)cc5ccn(CC6(F)CCNCC6)c45)ncnn3c2)c1=O ; Cl] | A* |
| 128. | 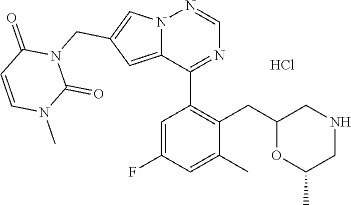 [SMILES: Cc1cc(F)cc(-c2ncnn3cc(Cn4c(=O)ccn(C)c4=O)cc23)c1CC1CNC[C@H](C)O1 ; Cl] | C* |
| 129. | 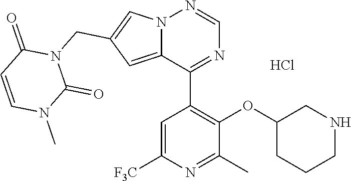 [SMILES: Cc1nc(C(F)(F)F)cc(-c2ncnn3cc(Cn4c(=O)ccn(C)c4=O)cc23)c1OC1CCCNC1 ; Cl] | D* |
| 130. | 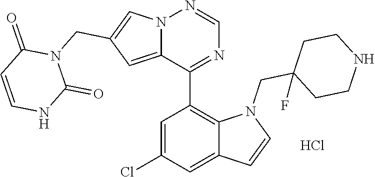 [SMILES: O=c1cc[nH]c(=O)n1Cc1cc2c(-c3cc(Cl)cc4ccn(CC5(F)CCNCC5)c34)ncnn2c1 ; Cl] | A |
| 131. | 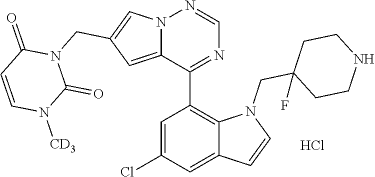 [SMILES: [2H]C([2H])([2H])n1ccc(=O)n(Cc2cc3c(-c4cc(Cl)cc5ccn(CC6(F)CCNCC6)c45)ncnn3c2)c1=O ; Cl] | A |
| 132. | 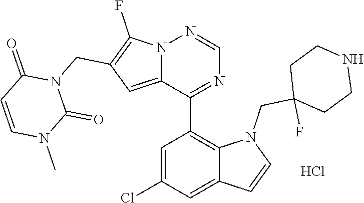 [SMILES: Cn1ccc(=O)n(Cc2cc3c(-c4cc(Cl)cc5ccn(CC6(F)CCNCC6)c45)ncnn3c2F)c1=O ; Cl] | |

### INCORPORATION BY REFERENCE

All U.S. patents, U.S. published patent applications, and PCT published patent applications designating the U.S. mentioned herein are hereby incorporated by reference in their entirety as if each individual publication or patent was specifically and individually indicated to be incorporated by reference. In case of conflict, the present application, including any definitions herein, will control.

### EQUIVALENTS

The foregoing written specification is sufficient to enable one skilled in the art to practice the invention. The present invention is not to be limited in scope by the examples provided, since each of the examples is intended as a single illustration of one aspect of the invention—other functionally equivalent embodiments are within the scope of the invention. Various modifications of the invention in addition to those shown and described herein will become apparent to those skilled in the art from the foregoing description and fall within the scope of the appended claims. All of the many advantages and objects of the invention are not necessarily encompassed by each embodiment of the invention.

What is claimed is:

1. A compound of structural Formula (I): 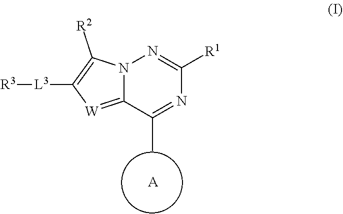
wherein:
R¹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R² represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R³ represents N-containing heterocyclyl, (N-containing heterocyclyl)-NH—, or (N-containing heterocyclyl)-O—;
or R³ represents —OH, —O—C₁-C₆ alkyl, —NH₂, —NH(—C₁-C₆ alkyl), or —N(—C₁-C₆ alkyl)₂;
L³ represents a single bond, C₁-C₃ alkylene, or —C(═O)—;
W represents C(—R⁴) or N;
if present, R⁴ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
and 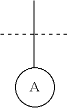
represents:
a) 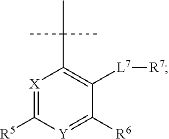
each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁶ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁷ represents N-containing heterocyclyl;
or R⁷ represents C₁-C₆ alkyl or H₂N—C₁-C₆ alkyl;
L⁷ represents a single bond, C₁-C₃ alkylene, —O—C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
b) 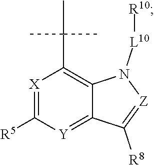
each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁸ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
Z represents C(—R⁹) or N;
if present, R⁹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R¹⁰ represents N-containing heterocyclyl;
or R¹⁰ represents —H or C₁-C₆ alkyl;
L¹⁰ represents a single bond, C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
c) 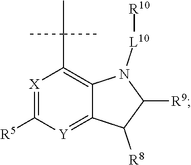
each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁸ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R¹⁰ represents N-containing heterocyclyl;
or R¹⁰ represents —H or C₁-C₆ alkyl;
L¹⁰ represents a single bond, C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
d) 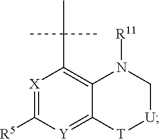
wherein each X and Y, independently, represents C(—R^{C});
each R^{C} represents —H;
R⁵ represents —Cl;
R¹¹ represents pyrrolidin-3-yl, piperidin-4-yl, morpholin-2-yl, or azetidin-3-yl; and
either T represents CH₂ or O, and U represents —CH₂— or CH(—CH₃),
or -T-U— represents 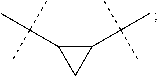
wherein each alkyl substituent independently represents a C₁-C₆ straight-chain alkyl or C₃-C₆ branched alkyl or C₃-C₆ cycloalkyl, as applicable, and is unsubstituted or substituted with one or more substituents, as valence permits, selected independently from the group consisting of deuterium, —F, —Cl, —Br, —I, —OH, —NH₂, —C(═O)—OH, heterocyclyl, and C₆-C₁₀ aryl;
wherein each N-containing heterocyclyl substituent independently represents a monocyclic or bicyclic non-aromatic ring system containing 4 to 9 ring member atoms including one or two nitrogen atoms and optionally one oxygen atom, and is optionally substituted with one or more substituents, as valence permits, selected independently from the group consisting of C₁-C₆ alkyl, —C≡N, —F, —Cl, —Br, —I, —CF₃, —OH, —NH₂, —C(═O)—OH, heterocyclyl, and C₆-C₁₀ aryl, and optionally one or two —CH₂— groups in the ring structure being replaced with —C(═O)— groups;
wherein each C₆-C₁₀ aryl substituent independently represents a monocyclic or bicyclic aromatic hydrocarbon ring system containing 6 to 10 ring member carbon atoms, and is unsubstituted or substituted with one or more substituents, as valence permits, selected independently from the group consisting of C₁-C₆ alkyl, —C≡N, —F, —Cl, —Br, —I, —OH, —NH₂, —C(═O)—OH, heterocyclyl, and C₆-C₁₀ aryl;
or a tautomer, stereoisomer, a racemic or scalemic mixture of stereoisomers, a pharmaceutically acceptable salt, ester, solvate, or polymorph thereof.

2. The compound according to claim 1, having the structural Formula (I): 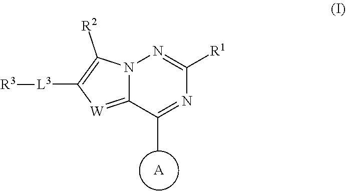
wherein:
R¹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R² represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R³ represents N-containing heterocyclyl, (N-containing heterocyclyl)-NH—, or (N-containing heterocyclyl)-O—;
or R³ represents —OH, —O—C₁-C₆ alkyl, —NH₂, —NH(—C₁-C₆ alkyl), or —N(—C₁-C₆ alkyl)₂; and
L³ represents a single bond, C₁-C₃ alkylene, or —C(═O)—;
W represents C(—R⁴) or N;
if present, R⁴ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
and 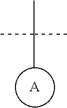
represents:
a) 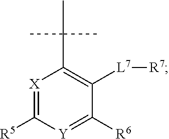
each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁶ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁷ represents N-containing heterocyclyl;
or R⁷ represents C₁-C₆ alkyl;
L⁷ represents a single bond, C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
b) 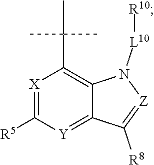
each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁸ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
Z represents C(—R⁹) or N;
if present, R⁹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R¹⁰ represents N-containing heterocyclyl;
or R¹⁰ represents —H or C₁-C₆ alkyl;
L¹⁰ represents a single bond, C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
or:
c) 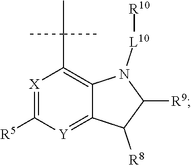
each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁸ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R⁹ represents —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R¹⁰ represents N-containing heterocyclyl;
or R¹⁰ represents —H or C₁-C₆ alkyl;
L¹⁰ represents a single bond, C₁-C₃ alkylene, —O—, —S—, —S(═O)—, —S(═O)₂—, —C(═O)—, —N(—R^{N})—, —N(—R^{N})—C(═O)—C(—R^{C})₂—, —N(—R^{N})—C(═O)—, —C(═O)—N(—R^{N})—, or —CH(—OH)—; and
if present, each occurrence of —R^{N} is independently selected from the group consisting of —H, C₁-C₆ alkyl, heterocyclyl, and C₆-C₁₀ aryl;
wherein each alkyl substituent independently represents a C₁-C₆ straight-chain alkyl or C₃-C₆ branched alkyl or C₃-C₆ cycloalkyl, as applicable, and is unsubstituted or substituted with one or more substituents, as valence permits, selected independently from the group consisting of —F, —Cl, —Br, —I, —OH, —NH₂, —C(═O)—OH, heterocyclyl, and C₆-C₁₀ aryl;
wherein each N-containing heterocyclyl substituent independently represents a monocyclic or bicyclic non-aromatic ring system containing 4 or 5 or 6 ring member atoms including one or two nitrogen atoms and optionally one oxygen atom, and is optionally substituted with one or more substituents, as valence permits, selected independently from the group consisting of C₁-C₆ alkyl, —C≡N, —F, —Cl, —Br, —I, —CF₃, —OH, —NH₂, —C(═O)—OH, heterocyclyl, and C₆-C₁₀ aryl, and optionally one or two —CH₂— groups in the ring structure being replaced with —C(═O)— groups;
wherein each C₆-C₁₀ aryl substituent independently represents a monocyclic or bicyclic aromatic hydrocarbon ring system containing 6 to 10 ring member carbon atoms, and is unsubstituted or substituted with one or more substituents, as valence permits, selected independently from the group consisting of C₁-C₆ alkyl, —C≡N, —F, —Cl, —Br, —I, —OH, —NH₂, —C(═O)—OH, heterocyclyl, and C₆-C₁₀ aryl;
or a tautomer, stereoisomer, a racemic or scalemic mixture of stereoisomers, a pharmaceutically acceptable salt, ester, solvate, or polymorph thereof.

3. The compound according to claim 1, having the structural Formula (I): 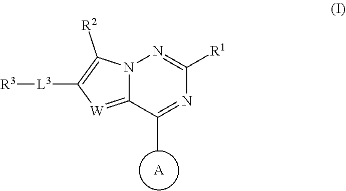
wherein:
R¹ represents —H or C₁-C₆ alkyl;
R² and R⁴, each independently, represent —H, C₁-C₆ alkyl, —C≡N, —N₃, —NO₂, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —OH, —O—C₁-C₆ alkyl, —C(═O)—OH, —C(═O)—NH₂, —C(═O)—C₁-C₆ alkyl, —C(═O)—O—C₁-C₆ alkyl, —C(═O)—NH—C₁-C₆ alkyl, —C(═O)—N(—C₁-C₆ alkyl)₂, —NH₂, —NH—C₁-C₆ alkyl, or —N(—C₁-C₆ alkyl)₂;
R³ represents N-containing heterocyclyl;
or R³ represents —OH, —O—C₁-C₆ alkyl, —NH₂, —NH(—C₁-C₆ alkyl), or —N(—C₁-C₆ alkyl)₂;
W represents C(—R⁴);
R⁴ represents —H or C₁-C₆ alkyl;
and 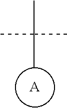
represents:
a) 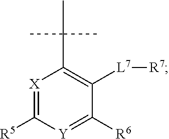
each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents C₁-C₆ alkyl, —C≡N, —N₃, —NO₂, —F, —Cl, —Br, or —CF₃;
R⁶ represents C₁-C₆ alkyl, —C≡N, —N₃, —NO₂, —F, —Cl, —Br, or —CF₃;
R⁷ represents N-containing heterocyclyl;
or R⁷ represents C₁-C₆ alkyl; and
L⁷ represents a single bond, C₁-C₃ alkylene, —O—, —S—, —S(═O)—, —S(═O)₂—, —C(═O)—, or —CH(—OH)—;
or:
b) 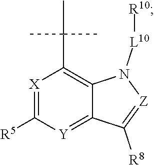
each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents C₁-C₆ alkyl, —C≡N, —N₃, —NO₂, —F, —Cl, —Br, —I, or —CF₃;
R⁸ represents —H, C₁-C₆ alkyl, —C≡N, —N₃, —NO₂, —F, —Cl, —Br, —I, or —CF₃;
Z represents C(—R⁹) or N;
if present, R⁹ represents —H, C₁-C₆ alkyl, —C≡N, —N₃, —NO₂, —F, —Cl, —Br, —I, or —CF₃;
R¹⁰ represents N-containing heterocyclyl;
or R¹⁰ represents —H or C₁-C₆ alkyl; and
L¹⁰ represents a single bond, C₁-C₃ alkylene, —O—, —S—, —S(═O)—, —S(═O)₂—, —C(═O)—, or —CH(—OH)—;
or:
c) 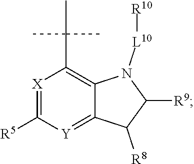
each X and Y, independently, represents C(—R^{C}) or N;
if present, each —R^{C} independently represents —H, C₁-C₆ alkyl, —C≡N, —F, —Cl, —Br, —I, or —CF₃;
R⁵ represents C₁-C₆ alkyl, —C≡N, —N₃, —NO₂, —F, —Cl, —Br, —I, or —CF₃;
R⁸ represents —H, C₁-C₆ alkyl, —C≡N, —N₃, —NO₂, —F, —Cl, —Br, —I, or —CF₃;
R⁹ represents —H, C₁-C₆ alkyl, —C≡N, —N₃, —NO₂, —F, —Cl, —Br, —I, or —CF₃;
R¹⁰ represents N-containing heterocyclyl;
or R¹⁰ represents —H or C₁-C₆ alkyl; and
L¹⁰ represents a single bond, C₁-C₃ alkylene, —O—, —S—, —S(═O)—, —S(═O)₂—, —C(═O)—, or —CH(—OH)—;
wherein each alkyl substituent independently represents C₁-C₆ straight-chain alkyl or C₃-C₆ branched alkyl or C₃-C₆ cycloalkyl, as applicable, and is unsubstituted or substituted with one or more substituents, as valence permits, selected independently from the group consisting of —F, —Cl, —Br, —I, —OH, —NH₂, —C(═O)—OH, heterocyclyl, and C₆-C₁₀ aryl;
wherein each N-containing heterocyclyl substituent independently represents a monocyclic or bicyclic non-aromatic ring system containing 4 or 5 or 6 ring member atoms including one or two nitrogen atoms and optionally one oxygen atom, and is optionally substituted with one or more substituents, as valence permits, selected independently from the group consisting of C₁-C₆ alkyl, —C≡N, —F, —Cl, —Br, —I, —CF₃, —OH, —NH₂, —C(═O)—OH, heterocyclyl, and C₆-C₁₀ aryl, and optionally one or two —CH₂— groups in the ring structure being replaced with —C(═O)— groups;
or a tautomer, stereoisomer, a racemic or scalemic mixture of stereoisomers, a pharmaceutically acceptable salt, ester, solvate, or polymorph thereof.

4. The compound according to claim 1, having the structural Formula (I): 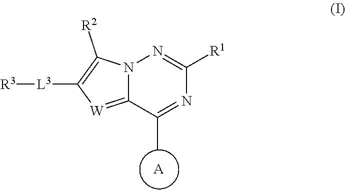
wherein:
R¹ represents —H;
R² represents —H;
R³ represents 6,6-dimethyl-2,4-dioxo-3-azabicyclo[3.1.0]hexan-3-yl, —OH, ethoxy, or —NH₂;
L³ represents —CH₂— or —C(═O)—;
W represents C(—R⁴);
R⁴ represents —H or —CH₃;
and 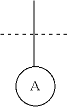
represents:
a) 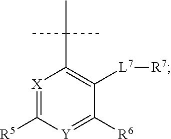
each X and Y, independently, represents C(—R^{C});
R^{C} represents —H;
R⁵ represents —Cl;
R⁶ represents methyl;
R⁷ represents piperidin-3-yl, morpholin-2-yl, 6-methylpiperidin-3-yl, piperidin-4-yl, 2-methylpiperazin-1-yl, 2-carboxypyrrolidin-4-yl, 3,3-difluoroazetidin-1-yl, 2-aminoethyl, 2,6-dimethylpiperazin-1-yl, 4-fluoropyrrolidin-3-yl, 5-methylpyrrolidin-3-yl, or 3-amino-4-methylpyrrolidin-1-yl; and
L⁷ represents —CH₂—, —O—, or —C(═O)—;
or:
b) 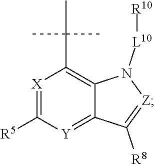
each X and Y, independently, represents C(—R^{C});
R^{C} represents —H;
R⁵ represents —Cl or —CF₃;
R⁸ represents —H;
Z represents C(—R⁹);
R⁹ represents —H;
R¹⁰ represents pyrrolidin-3-yl, morpholin-2-yl, 5,5-difluoropiperidin-3-yl, 4-fluoropiperidin-4-yl, 4-cyanopiperidin-4-yl, or 4-hydroxypiperidin-4-yl; and
L¹⁰ represents a single bond or —CH₂—;
or a tautomer, stereoisomer, a racemic or scalemic mixture of stereoisomers, a pharmaceutically acceptable salt, ester, solvate, or polymorph thereof.

5. The compound according to claim 1, wherein:
either:
a) R⁷ represents (S)-piperidin-3-yl, (R)-morpholin-2-yl, (3R,6R)-6-methylpiperidin-3-yl, piperidin-4-yl, (S)-2-methylpiperazin-1-yl, (2S,4S)-2-carboxypyrrolidin-4-yl, 3,3-difluoroazetidin-1-yl, 2-aminoethyl, (2S,6S)-2,6-dimethylpiperazin-1-yl, (3R,4S)-4-fluoropyrrolidin-3-yl, (3R,5R)-5-methylpyrrolidin-3-yl, or (3S,4S)-3-amino-4-methylpyrrolidin-1-yl; and
L⁷ represents —CH₂— or —O—;
or:
b) R¹⁰ represents (R)-pyrrolidin-3-yl, (S)-pyrrolidin-3-yl, 5,5-difluoropiperidin-3-yl, 4-fluoropiperidin-4-yl, 4-cyanopiperidin-4-yl, or 4-hydroxypiperidin-4-yl; and
L¹⁰ represents —CH₂—;
or a pharmaceutically acceptable salt, ester, solvate, or polymorph thereof.

6. The compound according to claim 1, wherein:
either:
a) R⁷ represents (S)-piperidin-3-yl, (R)-morpholin-2-yl, (S)-2-methylpiperazin-1-yl, (2S,6S)-2,6-dimethylpiperazin-1-yl, or (3R,4S)-4-fluoropyrrolidin-3-yl;
or:
b) R¹⁰ represents 4-fluoropiperidin-4-yl;
or a pharmaceutically acceptable salt, ester, solvate, or polymorph thereof.

7. The compound according to claim 1,
wherein:
R³ represents 6,6-dimethyl-2,4-dioxo-3-azabicyclo[3.1.0]hexan-3-yl; and
L³ represents —CH₂—;
or a pharmaceutically acceptable salt, ester, solvate, or polymorph thereof.

8. The compound according to claim 1,
wherein:
W represents C(—R⁴); and
R⁴ represents —H;
or a pharmaceutically acceptable salt, ester, solvate, or polymorph thereof.

9. The compound according to claim 1,
wherein:
R⁵ represents —Cl;
or a pharmaceutically acceptable salt, ester, solvate, or polymorph thereof.

10. The compound according to claim 1,
wherein:
R⁶ represents methyl;
or a pharmaceutically acceptable salt, ester, solvate, or polymorph thereof.

11. The compound according to claim 1, wherein the compound is:
3-((4-(5-chloro-3-methyl-2-(((S)-piperidin-3-yl)oxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((R)-morpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((3R,6R)-6-methylpiperidin-3-yl)oxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(piperidin-4-yloxy)phenyl)pyrrolo[2,1-f][1,2,4]triazin-6-yl)methyl)-6,6-dimethyl-3-azabicyclo[3.1.0]hexane-2,4-dione;
6,6-dimethyl-3-((4-(1-(pyrrolidin-3-yl)-5-(trifluoromethyl)-1H-indol-7-yl)pyrrolo[2,1-f][1,2,4]triazin-6-yl)methyl)-3-azabicyclo[3.1.0]hexane-2,4-dione;
3-((4-(5-chloro-3-methyl-2-(((S)-2-methylpiperazin-1-yl)methyl)phenyl)pyrrolo[2,1-f][1,2,4]triazin-6-yl)methyl)-6,6-dimethyl-3-azabicyclo[3.1.0]hexane-2,4-dione;
(2S,4S)-4-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenoxy)pyrrolidine-2-carboxylic acid;
3-((4-(5-chloro-2-(3,3-difluoroazetidine-1-carbonyl)-3-methylphenyl)pyrrolo[2,1-f][1,2,4]triazin-6-yl)methyl)-6,6-dimethyl-3-azabicyclo[3.1.0]hexane-2,4-dione;
3-((4-(2-(2-aminoethoxy)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-1-((4-fluoropiperidin-4-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(5-chloro-2-(((2S,6S)-2,6-dimethylpiperazin-1-yl)methyl)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((S)-piperidin-3-yl)oxy)phenyl)-5-methylpyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((R)-morpholin-2-yl)methyl)phenyl)-5-methylpyrrolo[2,1-f][1,2,4]triazin-6-yl)methyl)-6,6-dimethyl-3-azabicyclo[3.1.0]hexane-2,4-dione;
3-((4-(5-chloro-2-(((3R,4S)-4-fluoropyrrolidin-3-yl)oxy)-3-methylphenyl)pyrrolo[2,1-f][1,2,4]triazin-6-yl)methyl)-6,6-dimethyl-3-azabicyclo[3.1.0]hexane-2,4-dione;
ethyl (S)-4-(5-chloro-3-methyl-2-(piperidin-3-yloxy)phenyl)pyrrolo[2,1-f][1,2,4]triazine-6-carboxylate;
(S)-4-(5-chloro-3-methyl-2-(piperidin-3-yloxy)phenyl)pyrrolo[2,1-f][1,2,4]triazine-6-carboxylic acid;
(S)-(4-(5-chloro-3-methyl-2-(piperidin-3-yloxy)phenyl)pyrrolo[2,1-f][1,2,4]triazin-6-yl)methanol;
(S)-4-(5-chloro-3-methyl-2-(piperidin-3-yloxy)phenyl)pyrrolo[2,1-f][1,2,4]triazine-6-carboxamide;
3-((4-(1-((4-hydroxypiperidin-4-yl)methyl)-5-(trifluoromethyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((3R,5R)-5-methylpyrrolidin-3-yl)oxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-1-((4-fluoropiperidin-4-yl)methyl)-1H-indol-7-yl)-5-methylpyrrolo[2,1-f][1,2,4]triazin-6-yl)methyl)-6,6-dimethyl-3-azabicyclo[3.1.0]hexane-2,4-dione;
3-((4-(1-((4-fluoropiperidin-4-yl)methyl)-5-(trifluoromethyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(2-((3S,4S)-3-amino-4-methylpyrrolidine-1-carbonyl)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-1-(morpholin-2-ylmethyl)-1H-indol-7-yl)pyrrolo[2,1-f][1,2,4]triazin-6-yl)methyl)-6,6-dimethyl-3-azabicyclo[3.1.0]hexane-2,4-dione;
4-((5-chloro-7-(6-((6,6-dimethyl-2,4-dioxo-3-azabicyclo[3.1.0]hexan-3-yl)methyl)pyrrolo[2,1-f][1,2,4]triazin-4-yl)-1H-indol-1-yl)methyl)piperidine-4-carbonitrile; or
3-((4-(5-chloro-1-((5,5-difluoropiperidin-3-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
or a tautomer, stereoisomer, a racemic or scalemic mixture of stereoisomers, a pharmaceutically acceptable salt, ester, solvate, or polymorph thereof.

12. The compound according to claim 1, having the structural Formula (I): 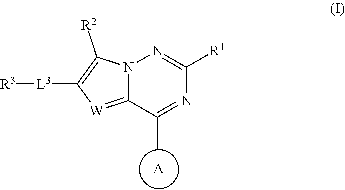
wherein:
R¹ represents —H;
R² represents —H;
R³ represents 6,6-dimethyl-2,4-dioxo-3-azabicyclo[3.1.0]hexan-3-yl, ethoxy, —OH, —NH₂, 2,4-dioxo-dihydropyrimidin-3-yl, 1-methyl-2,4-dioxo-dihydropyrimidin-3-yl, 1-methyl-2,4-dioxo-pyrimidin-3-yl, 1-(methyl-d₃)-2,4-dioxo-pyrimidin-3-yl, 1-ethyl-2,4-dioxo-pyrimidin-3-yl, 1-(2-fluoroethyl)-2,4-dioxo-pyrimidin-3-yl, 1-(2,2-difluoroethyl)-2,4-dioxo-pyrimidin-3-yl, 1-(2,2,2-trifluoroethyl)-2,4-dioxo-pyrimidin-3-yl, 1-isopropyl-2,4-dioxo-pyrimidin-3-yl, 1-cyclopropyl-2,4-dioxo-pyrimidin-3-yl, 5-fluoro-1-methyl-2,4-dioxo-pyrimidin-3-yl, 1,5-dimethyl-2,4-dioxo-pyrimidin-3-yl, 1-carboxymethyl-2,4-dioxo-dihydropyrimidin-3-yl, 1-methyl-2,4-dioxo-imidazolidin-3-yl, or 4-methyl-2,6-dioxo-piperazin-1-yl;
L³ represents —CH₂— or —C(═O)—;
W represents C(—R⁴);
R⁴ represents —H or —CH₃;
and 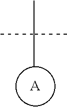
represents:
a) 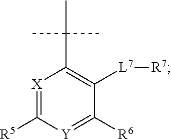
X represents C(—R^{C});
Y represents C(—R^{C}) or N;
R^{C} represents —H;
R⁵ represents —F, —Cl, or —CF₃;
R⁶ represents —H, —F, —Cl, or —CH₃;
R⁷ represents piperidin-3-yl, morpholin-2-yl, 6-methylpiperidin-3-yl, piperidin-4-yl, 2-methylpiperazin-1-yl, 2-carboxypyrrolidin-4-yl, 3,3-difluoroazetidin-1-yl, 2-aminoethyl, 2,6-dimethylpiperazin-1-yl, 4-fluoropyrrolidin-3-yl, 5-methylpyrrolidin-3-yl, 3-amino-4-methylpyrrolidin-1-yl, 6-methylmorpholin-2-yl, 5,5-difluoropiperidin-3-yl, 6,6-difluoro-1,4-oxepan-2-yl, 5-methylmorpholin-2-yl, 6-fluoromethylmorpholin-2-yl, 4-oxa-7-azaspiro[2.5]octan-5-yl, 6,6-dimethylmorpholin-2-yl, 4-methylpiperidin-4-yl, 7-oxa-4-azaspiro[2.5]octan-6-yl, 2,6,6-trimethylmorpholin-2-yl, 3-methylpiperidin-3-yl, 4-fluoropiperidin-4-yl, 1,2,3,6-tetrahydropyridin-4-yl, piperazin-1-yl, 3-aminopyrrolidin-1-yl, hexahydropyrrolo[3,4-b]pyrrol-5(1H)-yl, 1,7-diazaspiro[4,4]nonan-7-yl, hexahydropyrrolo[3,4-c]pyrrol-2(1H)-yl, 2,7-diazaspiro[3.5]nonan-7-yl, 4-methylmorpholin-2-yl, or 3-(methylamino)piperidin-1-yl; and
L⁷ represents a single bond, —CH₂—, —O—, —O—CH₂—, —O—CH(—CH₃)— or —C(═O)—;
or a tautomer, stereoisomer, a racemic or scalemic mixture of stereoisomers, a pharmaceutically acceptable salt, ester, solvate, or polymorph thereof.

13. The compound according to claim 1, wherein the compound is:
3-((4-(5-chloro-3-methyl-2-(((S)-piperidin-3-yl)oxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((R)-morpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((3R,6R)-6-methylpiperidin-3-yl)oxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(piperidin-4-yloxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((S)-2-methylpiperazin-1-yl)methyl)phenyl)pyrrolo[2,1-f][1,2,4]triazin-6-yl)methyl)-6,6-dimethyl-3-azabicyclo[3.1.0]hexane-2,4-dione;
(2S,4S)-4-(4-chloro-2-(6-((6,6-dimethyl-2,4-dioxo-3-azabicyclo[3.1.0]hexan-3-yl)methyl)pyrrolo[2,1-f][1,2,4]triazin-4-yl)-6-methylphenoxy)pyrrolidine-2-carboxylic acid;
3-((4-(5-chloro-2-(3,3-difluoroazetidine-1-carbonyl)-3-methylphenyl)pyrrolo[2,1-f][1,2,4]triazin-6-yl)methyl)-6,6-dimethyl-3-azabicyclo[3.1.0]hexane-2,4-dione;
3-((4-(2-(2-aminoethoxy)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-2-(((2S,6S)-2,6-dimethylpiperazin-1-yl)methyl)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((S)-piperidin-3-yl)oxy)phenyl)-5-methylpyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((R)-morpholin-2-yl)methyl)phenyl)-5-methylpyrrolo[2,1-f][1,2,4]triazin-6-yl)methyl)-6,6-dimethyl-3-azabicyclo[3.1.0]hexane-2,4-dione;
3-((4-(5-chloro-2-(((3R,4S)-4-fluoropyrrolidin-3-yl)oxy)-3-methylphenyl)pyrrolo[2,1-f][1,2,4]triazin-6-yl)methyl)-6,6-dimethyl-3-azabicyclo[3.1.0]hexane-2,4-dione;
ethyl (S)-4-(5-chloro-3-methyl-2-(piperidin-3-yloxy)phenyl)pyrrolo[2,1-f][1,2,4]triazine-6-carboxylate;
(S)-4-(5-chloro-3-methyl-2-(piperidin-3-yloxy)phenyl)pyrrolo[2,1-f][1,2,4]triazine-6-carboxylic acid;
(S)-(4-(5-chloro-3-methyl-2-(piperidin-3-yloxy)phenyl)pyrrolo[2,1-f][1,2,4]triazin-6-yl)methanol;
(S)-4-(5-chloro-3-methyl-2-(piperidin-3-yloxy)phenyl)pyrrolo[2,1-f][1,2,4]triazine-6-carboxamide;
3-((4-(5-chloro-3-methyl-2-(((3S,5S)-5-methylpyrrolidin-3-yl)oxy)phenyl)pyrrolo[2,1-f][1,2,4]triazin-6-yl)methyl)-6,6-dimethyl-3-azabicyclo[3.1.0]hexane-2,4-dione;
3-((4-(2-((3S,4S)-3-amino-4-methylpyrrolidine-1-carbonyl)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((6S)-6-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-(((S)-5,5-difluoropiperidin-3-yl)oxy)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-2-((6,6-difluoro-1,4-oxazepan-2-yl)methyl)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((5R)-5-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((5R)-5-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-((6-(fluoromethyl)morpholin-2-yl)methyl)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-2-((6-(fluoromethyl)morpholin-2-yl)methyl)-3-methylphenyl)pyrrolo[2,1-f][1,2,4]triazin-6-yl)methyl)-6,6-dimethyl-3-azabicyclo[3.1.0]hexane-2,4-dione;
3-((4-(2-((4-oxa-7-azaspiro[2.5]octan-5-yl)methyl)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(piperidin-4-ylmethoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((5S)-5-methylmorpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-((6,6-dimethylmorpholin-2-yl)methyl)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((R)-morpholin-2-yl)methoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((R)-piperidin-3-yl)methoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(1-(piperidin-4-yl)ethoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(1-(piperidin-4-yl)ethoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((4-methylpiperidin-4-yl)methoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione;
3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-(2,2,2-trifluoroethyl)pyrimidine-2,4(1H,3H)-dione;
3-((4-(5-chloro-3-methyl-2-(morpholin-2-ylmethyl)phenyl)pyrrolo[2,1-f][1,2,4]triazin-6-yl)methyl)-1-(2,2,2-trifluoroethyl)pyrimidine-2,4(1H,3H)-dione;
3-((4-(2-((7-oxa-4-azaspiro[2.5]octan-6-yl)methyl)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((2,6,6-trimethylmorpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((piperidin-4-ylmethyl)amino)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((3-methylpiperidin-3-yl)methoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((3-methylpiperidin-3-yl)methoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((R)-1-((S)-morpholin-2-yl)ethoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((R)-1-((S)-morpholin-2-yl)ethoxy)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((4-methylpiperidin-4-yl)methyl)amino)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((piperidin-3-ylmethyl)amino)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-(((4-fluoropiperidin-4-yl)methyl)amino)-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-((((S)-morpholin-2-yl)methyl)amino)phenyl)pyrrolo[2,1-f][1,2,4]triazin-6-yl)methyl)-6,6-dimethyl-3-azabicyclo[3.1.0]hexane-2,4-dione;
(R)-3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-(2,2,2-trifluoroethyl)pyrimidine-2,4(1H,3H)-dione;
3-((4-(5-chloro-3-methyl-2-(1,2,3,6-tetrahydropyridin-4-yl)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-(piperazin-1-yl)phenyl)pyrrolo[2,1-f][1,2,4]triazin-6-yl)methyl)-6,6-dimethyl-3-azabicyclo[3.1.0]hexane-2,4-dione;
3-((4-(2-((S)-3-aminopyrrolidin-1-yl)-5-chlorophenyl)pyrrolo[2,1-f][1,2,4]triazin-6-yl)methyl)-6,6-dimethyl-3-azabicyclo[3.1.0]hexane-2,4-dione;
3-((4-(2-((R)-3-aminopyrrolidin-1-yl)-5-chlorophenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(1,2,3,6-tetrahydropyridin-4-yl)phenyl)pyrrolo[2,1-f][1,2,4]triazin-6-yl)methyl)-1-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-isopropylpyrimidine-2,4(1H,3H)-dione;
3-((4-(5-chloro-2-((3aR,6aR)-hexahydropyrrolo[3,4-b]pyrrol-5(1H)-yl)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-(1,7-diazaspiro[4.4]nonan-7-yl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-(2,2-difluoroethyl)pyrimidine-2,4(1H,3H)-dione;
3-((4-(5-chloro-2-((3aS,6aS)-hexahydropyrrolo[3,4-b]pyrrol-5(1H)-yl)phenyl)pyrrolo[2,1-f][1,2,4]triazin-6-yl)methyl)-6,6-dimethyl-3-azabicyclo[3.1.0]hexane-2,4-dione;
3-((4-(5-chloro-2-(hexahydropyrrolo[3,4-c]pyrrol-2(1H)-yl)phenyl)pyrrolo[2,1-f][1,2,4]triazin-6-yl)methyl)-6,6-dimethyl-3-azabicyclo[3.1.0]hexane-2,4-dione;
(R)-1-((4-(5-chloro-3-methyl-2-(morpholin-2-ylmethyl)phenyl)pyrrolo[2,1-f][1,2,4]triazin-6-yl)methyl)-5-fluoro-3-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-methylimidazolidine-2,4-dione;
(S)-3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-(2-fluoroethyl)pyrimidine-2,4(1H,3H)-dione;
3-((4-(2-((2,7-diazaspiro[3.5]nonan-7-yl)methyl)-5-chloro-3-methylphenyl)pyrrolo[2,1-f][1,2,4]triazin-6-yl)methyl)-6,6-dimethyl-3-azabicyclo[3.1.0]hexane-2,4-dione;
3-((4-(5-chloro-3-fluoro-2-((4-methylmorpholin-2-yl)methyl)phenyl)pyrrolo[2,1-f][1,2,4]triazin-6-yl)methyl)-1-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-ethylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-(methyl-d₃)pyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-cyclopropylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-cyclopropylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-5-fluoro-1-methylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-methyldihydropyrimidine-2,4(1H,3H)-dione;
3-((4-(5-chloro-3-methyl-2-(((5S)-5-methylmorpholin-2-yl)methyl)phenyl)pyrrolo[2,1-f][1,2,4]triazin-6-yl)methyl)-1-methylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-isopropylpyrimidine-2,4(1H,3H)-dione;
3-((4-(5-chloro-2-((S)-3-(methylamino)piperidin-1-yl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((5S)-5-methylmorpholin-2-yl)methyl)phenyl)pyrrolo[2,1-f][1,2,4]triazin-6-yl)methyl)-1-isopropylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-ethylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1,5-dimethylpyrimidine-2,4(1H,3H)-dione;
3-((4-(5-chloro-3-fluoro-2-(morpholin-2-ylmethyl)phenyl)pyrrolo[2,1-f][1,2,4]triazin-6-yl)methyl)-1-methylpyrimidine-2,4(1H,3H)-dione;
1-((4-(5-chloro-3-methyl-2-(((6S)-6-methylmorpholin-2-yl)methyl)phenyl)pyrrolo[2,1-f][1,2,4]triazin-6-yl)methyl)-4-methylpiperazine-2,6-dione;
3-((4-(3,5-dichloro-2-(morpholin-2-ylmethyl)phenyl)pyrrolo[2,1-f][1,2,4]triazin-6-yl)methyl)-1-methylpyrimidine-2,4(1H,3H)-dione;
1-((4-(5-chloro-3-fluoro-2-(((6S)-6-methylmorpholin-2-yl)methyl)phenyl)pyrrolo[2,1-f][1,2,4]triazin-6-yl)methyl)-4-methylpiperazine-2,6-dione;
3-((4-(5-chloro-3-fluoro-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)pyrimidine-2,4(1H,3H)-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)pyrimidine-2,4(1H,3H)-dione;
(R)-2-(3-((4-(5-chloro-3-methyl-2-(morpholin-2-ylmethyl)phenyl)pyrrolo[2,1-f][1,2,4]triazin-6-yl)methyl)-2,4-dioxo-3,4-dihydropyrimidin-1(2H)-yl)acetic acid;
3-((4-(3,5-dichloro-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione;
3-((4-(5-fluoro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione; or
1-methyl-3-((4-(2-methyl-3-(piperidin-3-yloxy)-6-(trifluoromethyl)pyridin-4-yl)pyrrolo[2,1-f][1,2,4]triazin-6-yl)methyl)pyrimidine-2,4(1H,3H)-dione;
or a tautomer, stereoisomer, a racemic or scalemic mixture of stereoisomers, a pharmaceutically acceptable salt, ester, solvate, or polymorph thereof.

14. The compound according to claim 1, wherein the compound is:
3-((4-(5-chloro-3-methyl-2-(((R)-morpholin-2-yl)methyl)phenyl)pyrrolo[2,1-f][1,2,4]triazin-6-yl)methyl)-6,6-dimethyl-3-azabicyclo[3.1.0]hexane-2,4-dione;
3-((4-(5-chloro-3-methyl-2-(((6S)-6-methylmorpholin-2-yl)methyl)phenyl)pyrrolo[2,1-f][1,2,4]triazin-6-yl)methyl)-1-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-methylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-isopropylpyrimidine-2,4(1H,3H)-dione;
(R)-3-((4-(5-chloro-3-methyl-2-(morpholin-2-ylmethyl)phenyl)pyrrolo[2,1-f][1,2,4]triazin-6-yl)methyl)-1-(methyl-d₃)pyrimidine-2,4(1H,3H)-dione; or
3-((4-(5-chloro-3-methyl-2-(((6S)-6-methylmorpholin-2-yl)methyl)phenyl)pyrrolo[2,1-f][1,2,4]triazin-6-yl)methyl)-1-isopropylpyrimidine-2,4(1H,3H)-dione;
or a tautomer, stereoisomer, a racemic or scalemic mixture of stereoisomers, a pharmaceutically acceptable salt, ester, solvate, or polymorph thereof.

15. The compound according to claim 1, having the structural Formula (I): 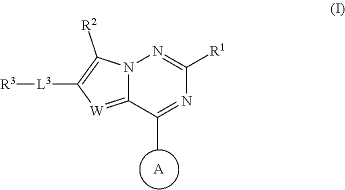
wherein:
R¹ represents —H or —Cl;
R² represents —H or —F;
R³ represents 6,6-dimethyl-2,4-dioxo-3-azabicyclo[3.1.0]hexan-3-yl, 1,3-dioxo-tetrahydropyrrolo[1,2-a]pyrazin-2-yl, 5,5-dimethyl-2,4-dioxo-imidazolidin-3-yl, 2,4-dioxo-imidazolidin-3-yl, 1-methyl-2,4-dioxo-pyrimidin-3-yl, 1-(2,2,2-trifluoroethyl)-2,4-dioxo-pyrimidin-3-yl, 2,4-dioxo-pyrimidin-3-yl, 1-isopropyl-2,4-dioxo-pyrimidin-3-yl, or 1-(methyl-d₃)-2,4-dioxo-pyrimidin-3-yl;
L³ represents —CH₂—;
W represents C(—R⁴);
R⁴ represents —H or —CH₃;
and 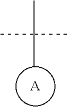
represents:
b) 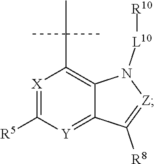
each X and Y, independently, represents C(—R^{C});
R^{C} represents —H;
R⁵ represents —F, —Cl, —CF₃, —NO₂, carboxy, or methoxycarbonyl;
R⁸ represents —H or —F;
Z represents C(—R⁹) or N;
if exists, R⁹ represents —H;
R¹⁰ represents pyrrolidin-3-yl, 4-fluoropiperidin-4-yl, 4-hydroxypiperidin-4-yl, morpholin-2-yl, 4-cyanopiperidin-4-yl, 5,5-difluoropiperidin-3-yl, or 5-fluoropiperidin-3-yl; and
L¹⁰ represents a single bond or —CH₂—;
or a tautomer, stereoisomer, a racemic or scalemic mixture of stereoisomers, a pharmaceutically acceptable salt, ester, solvate, or polymorph thereof.

16. The compound according to claim 1, wherein the compound is:
6,6-dimethyl-3-((4-(1-(pyrrolidin-3-yl)-5-(trifluoromethyl)-1H-indol-7-yl)pyrrolo[2,1-f][1,2,4]triazin-6-yl)methyl)-3-azabicyclo[3.1.0]hexane-2,4-dione;
3-((4-(5-chloro-1-((4-fluoropiperidin-4-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(1-((4-hydroxypiperidin-4-yl)methyl)-5-(trifluoromethyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(5-chloro-1-((4-fluoropiperidin-4-yl)methyl)-1H-indol-7-yl)-5-methylpyrrolo[2,1-f][1,2,4]triazin-6-yl)methyl)-6,6-dimethyl-3-azabicyclo[3.1.0]hexane-2,4-dione;
3-((4-(1-((4-fluoropiperidin-4-yl)methyl)-5-(trifluoromethyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(5-chloro-1-(morpholin-2-ylmethyl)-1H-indol-7-yl)pyrrolo[2,1-f][1,2,4]triazin-6-yl)methyl)-6,6-dimethyl-3-azabicyclo[3.1.0]hexane-2,4-dione;
4-((5-chloro-7-(6-((6,6-dimethyl-2,4-dioxo-3-azabicyclo[3.1.0]hexan-3-yl)methyl)pyrrolo[2,1-f][1,2,4]triazin-4-yl)-1H-indol-1-yl)methyl)piperidine-4-carbonitrile;
3-((4-(5-chloro-1-((5,5-difluoropiperidin-3-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(5-chloro-1-((3-fluoropiperidin-3-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
3-((4-(5-chloro-1-(((S)-morpholin-2-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
(S)-2-((4-(5-chloro-1-(((S)-morpholin-2-yl)methyl)-1H-indol-7-yl)pyrrolo[2,1-f][1,2,4]triazin-6-yl)methyl)tetrahydropyrrolo[1,2-a]pyrazine-1,3(2H,4H)-dione;
6,6-dimethyl-3-((4-(1-(((S)-morpholin-2-yl)methyl)-5-nitro-1H-indol-7-yl)pyrrolo[2,1-f][1,2,4]triazin-6-yl)methyl)-3-azabicyclo[3.1.0]hexane-2,4-dione;
3-((4-(5-fluoro-1-(((S)-morpholin-2-yl)methyl)-1H-indol-7-yl)pyrrolo[2,1-f][1,2,4]triazin-6-yl)methyl)-6,6-dimethyl-3-azabicyclo[3.1.0]hexane-2,4-dione;
methyl 7-(6-((6,6-dimethyl-2,4-dioxo-3-azabicyclo[3.1.0]hexan-3-yl)methyl)pyrrolo[2,1-f][1,2,4]triazin-4-yl)-1-(((S)-morpholin-2-yl)methyl)-1H-indole-5-carboxylate;
6,6-dimethyl-3-((4-(1-(((S)-morpholin-2-yl)methyl)-5-(trifluoromethyl)-1H-indol-7-yl)pyrrolo[2,1-f][1,2,4]triazin-6-yl)methyl)-3-azabicyclo[3.1.0]hexane-2,4-dione;
(S)-3-((4-(5-chloro-1-(morpholin-2-ylmethyl)-1H-indol-7-yl)pyrrolo[2,1-f][1,2,4]triazin-6-yl)methyl)-5,5-dimethylimidazolidine-2,4-dione;
(S)-3-((4-(5-chloro-1-(morpholin-2-ylmethyl)-1H-indol-7-yl)pyrrolo[2,1-f][1,2,4]triazin-6-yl)methyl)imidazolidine-2,4-dione;
(S)-3-((4-(5-chloro-1-(morpholin-2-ylmethyl)-1H-indol-7-yl)pyrrolo[2,1-f][1,2,4]triazin-6-yl)methyl)-1-methylpyrimidine-2,4(1H,3H)-dione;
3-((4-(5-chloro-1-(((S)-morpholin-2-yl)methyl)-1H-indazol-7-yl)pyrrolo[2,1-f][1,2,4]triazin-6-yl)methyl)-6,6-dimethyl-3-azabicyclo[3.1.0]hexane-2,4-dione;
3-((4-(5-chloro-3-fluoro-1-(((S)-morpholin-2-yl)methyl)-1H-indazol-7-yl)pyrrolo[2,1-f][1,2,4]triazin-6-yl)methyl)-6,6-dimethyl-3-azabicyclo[3.1.0]hexane-2,4-dione;
7-(6-((6,6-dimethyl-2,4-dioxo-3-azabicyclo[3.1.0]hexan-3-yl)methyl)pyrrolo[2,1-f][1,2,4]triazin-4-yl)-1-(((S)-morpholin-2-yl)methyl)-1H-indole-5-carboxylic acid;
3-((4-(5-chloro-1-((4-fluoropiperidin-4-yl)methyl)-1H-indol-7-yl)pyrrolo[2,1-f][1,2,4]triazin-6-yl)methyl)-1-methylpyrimidine-2,4(1H,3H)-dione;
3-((4-(5-chloro-1-((4-fluoropiperidin-4-yl)methyl)-1H-indol-7-yl)pyrrolo[2,1-f][1,2,4]triazin-6-yl)methyl)-1-(2,2,2-trifluoroethyl)pyrimidine-2,4(1H,3H)-dione;
3-((2-chloro-4-(5-chloro-1-((4-fluoropiperidin-4-yl)methyl)-1H-indol-7-yl)pyrrolo[2,1-f][1,2,4]triazin-6-yl)methyl)pyrimidine-2,4(1H,3H)-dione;
3-((4-(5-chloro-1-((4-fluoropiperidin-4-yl)methyl)-1H-indol-7-yl)pyrrolo[2,1-f][1,2,4]triazin-6-yl)methyl)-1-isopropylpyrimidine-2,4(1H,3H)-dione;
3-((4-(5-chloro-1-((4-fluoropiperidin-4-yl)methyl)-1H-indol-7-yl)pyrrolo[2,1-f][1,2,4]triazin-6-yl)methyl)pyrimidine-2,4(1H,3H)-dione;
3-((4-(5-chloro-1-((4-fluoropiperidin-4-yl)methyl)-1H-indol-7-yl)pyrrolo[2,1-f][1,2,4]triazin-6-yl)methyl)-1-(methyl-d₃)pyrimidine-2,4(1H,3H)-dione; or 3-((4-(5-chloro-1-((4-fluoropiperidin-4-yl)methyl)-1H-indol-7-yl)-7-fluoropyrrolo[2,1-f][1,2,4]triazin-6-yl)methyl)-1-methylpyrimidine-2,4(1H,3H)-dione;
or a tautomer, stereoisomer, a racemic or scalemic mixture of stereoisomers, a pharmaceutically acceptable salt, ester, solvate, or polymorph thereof.

17. The compound according to claim 1, wherein the compound is:
3-((4-(5-chloro-1-((4-fluoropiperidin-4-yl)methyl)-1H-indol-7-yl)pyrrolo[2,1-f][1,2,4]triazin-6-yl)methyl)-1-methylpyrimidine-2,4(1H,3H)-dione;
3-((4-(5-chloro-1-((4-fluoropiperidin-4-yl)methyl)-1H-indol-7-yl)pyrrolo[2,1-f][1,2,4]triazin-6-yl)methyl)-1-isopropylpyrimidine-2,4(1H,3H)-dione; or
3-((4-(5-chloro-1-((4-fluoropiperidin-4-yl)methyl)-1H-indol-7-yl)pyrrolo[2,1-f][1,2,4]triazin-6-yl)methyl)-1-(methyl-d₃)pyrimidine-2,4(1H,3H)-dione;
or a tautomer, stereoisomer, a racemic or scalemic mixture of stereoisomers, a pharmaceutically acceptable salt, ester, solvate, or polymorph thereof.

18. The compound according to claim 1, having the structural Formula (I): 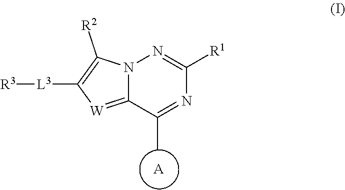
wherein:
R¹ represents —H;
R² represents —H;
R³ represents 6,6-dimethyl-2,4-dioxo-3-azabicyclo[3.1.0]hexan-3-yl;
L³ represents —CH₂—;
W represents C(—R⁴);
R⁴ represents —H;
and 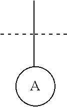
represents:
d) 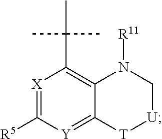
wherein each X and Y, independently, represents C(—R^{C});
each R^{C} represents —H;
R⁵ represents —Cl;
R¹¹ represents pyrrolidin-3-yl, piperidin-4-yl, morpholin-2-yl, or azetidin-3-yl; and
either T represents CH₂ or O, and U represents —CH₂— or CH(—CH₃),
or -T-U— represents 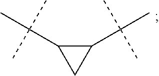
or a tautomer, stereoisomer, a racemic or scalemic mixture of stereoisomers, a pharmaceutically acceptable salt, ester, solvate, or polymorph thereof.

19. The compound according to claim 1, wherein the compound is:
3-((4-(6-chloro-1-(pyrrolidin-3-yl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(6-chloro-1-(piperidin-4-yl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(7-chloro-4-(pyrrolidin-3-yl)-3,4-dihydro-2H-benzo[b][1,4]oxazin-5-yl)pyrrolo[2,1-f][1,2,4]triazin-6-yl)methyl)-6,6-dimethyl-3-azabicyclo[3.1.0]hexane-2,4-dione;
3-((4-(6-chloro-1-((4-fluoropiperidin-4-yl)methyl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(6-chloro-1-(pyrrolidin-3-yl)-1,2,3,4-tetrahydroquinolin-8-yl)pyrrolo[2,1-f][1,2,4]triazin-6-yl)methyl)-1-(2,2,2-trifluoroethyl)pyrimidine-2,4(1H,3H)-dione;
3-((4-(6-chloro-1-(((S)-morpholin-2-yl)methyl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(6-chloro-1-(pyrrolidin-3-yl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(6-chloro-1-(pyrrolidin-3-yl)-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(1-(azetidin-3-yl)-6-chloro-1,2,3,4-tetrahydroquinolin-8-yl)pyrrolo[2,1-f][1,2,4]triazin-6-yl)methyl)-6,6-dimethyl-3-azabicyclo[3.1.0]hexane-2,4-dione;
3-((4-(6-chloro-3-(pyrrolidin-3-yl)-1a,2,3,7b-tetrahydro-1H-cyclopropa[c]quinolin-4-yl)pyrrolo[2,1-f][1,2,4]triazin-6-yl)methyl)-6,6-dimethyl-3-azabicyclo[3.1.0]hexane-2,4-dione;
3-((4-((2R)-7-chloro-2-methyl-4-(pyrrolidin-3-yl)-3,4-dihydro-2H-benzo[b][1,4]oxazin-5-yl)pyrrolo[2,1-f][1,2,4]triazin-6-yl)methyl)-6,6-dimethyl-3-azabicyclo[3.1.0]hexane-2,4-dione; or
3-((4-((2S)-7-chloro-2-methyl-4-(pyrrolidin-3-yl)-3,4-dihydro-2H-benzo[b][1,4]oxazin-5-yl)pyrrolo[2,1-f][1,2,4]triazin-6-yl)methyl)-6,6-dimethyl-3-azabicyclo[3.1.0]hexane-2,4-dione;
or a tautomer, stereoisomer, a racemic or scalemic mixture of stereoisomers, a pharmaceutically acceptable salt, ester, solvate, or polymorph thereof.

20. A pharmaceutical composition comprising (i) a therapeutically effective amount of at least one compound according to claim 1, or a pharmaceutically acceptable salt, solvate, or polymorph thereof; and (ii) a pharmaceutically acceptable carrier, vehicle or excipient therefor.

21. A method for inhibiting USP7 in a cell or a tissue, comprising contacting the cell or the tissue with at least one compound according to claim 1, or a pharmaceutically acceptable salt, solvate, or polymorph thereof, or with a pharmaceutical composition comprising a compound according to claim 1.

22. A method for the treatment or prevention of a disease, disorder, or condition associated with aberrant expression or activity of USP7, comprising administering to the subject in need thereof a therapeutically effective amount of at least one compound of claim 1, or a pharmaceutically acceptable salt, solvate, or polymorph thereof, or a pharmaceutical composition comprising a compound according to claim 1.

23. A method according to claim 22, wherein the disease, disorder, or condition is selected from the group consisting of cardiovascular disorders, pulmonary disorders, autoimmune disorders, immune disorders, immunoregulatory disorders, neurodegenerative disorders, metabolic disorders, hemolytic disorders, gastrointestinal disorders, sexual disorders, infections, wound healing disorders, and cancers.

24. A method according to claim 23, wherein the disease, disorder, or condition is a cardiovascular disorder selected from the group consisting of systemic hypertension, pulmonary arterial hypertension (PAH), pulmonary arterial hypertension in high altitude, ischemia reperfusion (IR) injury, myocardial infarction, and atherosclerosis.

25. A compound for use according to claim 24, wherein the cardiovascular disorder is pulmonary arterial hypertension (PAH).

26. A method according to claim 24, wherein the cardiovascular disorder is ischemia reperfusion (IR) injury selected from the group consisting of liver IR, kidney IR, and myocardial IR.

27. A according to claim 24, wherein the cardiovascular disorder is myocardial infarction or atherosclerosis.

28. A method according to claim 23, wherein the disease, disorder, or condition is a pulmonary disorder selected from the group consisting of chemically-induced lung fibrosis, idiopathic pulmonary fibrosis, cystic fibrosis, chronic obstructive pulmonary disease (COPD), and asthma.

29. A method according to claim 23, wherein the disease, disorder, or condition is an autoimmune disorder selected from the group consisting of encephalomyelitis, multiple sclerosis, anti-phospholipid syndrome 1, autoimmune hemolytic anemia, chronic inflammatory demyelinating polyradiculoneuropathy, psoriasis, dermatitis herpetiformis, dermatomyositis, myasthenia gravis, pemphigus, rheumatoid arthritis, stiff-person syndrome, type 1 diabetes, ankylosing spondylitis, paroxysmal nocturnal hemoglobinuria (PNH), paroxysmal cold hemoglobinuria, severe idiopathic autoimmune hemolytic anemia, and Goodpasture's syndrome.

30. A method according to claim 23, wherein the disease, disorder, or condition is an immune disorder selected from the group consisting of T-cell dysfunction mediated by myeloid-derived suppressor cells (MDSC), human immunodeficiency virus (HIV) infection, autoimmune encephalomyelitis, and ABO mismatch transfusion reaction.

31. A method according to claim 30, wherein the immune disorder is T-cell dysfunction mediated by myeloid-derived suppressor cells (MDSC).

32. A method according to claim 23, wherein the disease, disorder, or condition is a disease resulting from an immunoregulatory disorder selected from the group consisting of renal disease inflammation, hepatic fibrosis, leishmaniosis, neurodegenerative diseases, wound healing, human immunodeficiency virus (HIV) infection, hepatitis B virus (HBV) infection, Helicobacter pylori infection, fibrotic disorders, arthritis, candidiasis, periodontal disease, keloids, adenotonsilar disease, African sleeping sickness, Chagas' disease, and transplant rejection.

33. A method according to claim 23, wherein the disease, disorder, or condition is a neurodegenerative disorder selected from the group consisting of Alzheimer's disease, Parkinson's disease, Huntington's disease, extrapyramidal syndrome, dystonia, akathisia, epilepsy, periodic limb movement, and dementia.

34. A method according to claim 23, wherein the disease, disorder, or condition is a metabolic disorder selected from the group consisting of diabetes, non-alcoholic steatohepatitis (NASH), and non-alcoholic fatty liver disease (NAFLD).

35. A method according to claim 23, wherein the disease, disorder, or condition is a hemolytic disorder selected from the group consisting of sickle-cell disease, thalassemias, hereditary spherocytosis, stomatocytosis, microangiopathic hemolytic anemias, pyruvate kinase deficiency, infection-induced anemia, cardiopulmonary bypass, mechanical heart valve-induced anemia, and chemical-induced anemia.

36. A method according to claim 35, wherein the hemolytic disorder is sickle-cell disease.

37. A method according to claim 23, wherein the disease, disorder, or condition is a gastrointestinal disorder selected from the group consisting of gastrointestinal motility disorders, gastric cancers, inflammatory bowel disease, Crohn's disease, ulcerative colitis, and gastric ulcers.

38. A method according to claim 23, wherein the disease, disorder, or condition is a sexual disorder selected from the group consisting of Peyronie's disease, and erectile dysfunction.

39. A method according to claim 23, wherein the disease, disorder, or condition is a wound healing disorder selected from the group consisting of infected and uninfected wound healing.

40. A method according to claim 23, wherein the disease, disorder, or condition is a cancer selected from the group consisting of oesophagic, gastric, colon, ovary, breast, pancreatic, head-and-neck, bladder, and lung cancers (including squamous and non-small cell lung carcinoma), renal cell carcinoma, prostate carcinoma, multiple myeloma, neuroblastoma, glioblastoma, astrocytoma, mesothelioma and melanoma, B cells, T cells and NK cells lymphomas, acute and chronic, myeloid leukemia, and lymphoid leukemia.

41. A method according to claim 23, wherein the disease, disorder, or condition is a cancer selected from the group consisting of gastric cancer (including, but not limited to, gastric or gastroesophageal junction cancer), colorectal cancer, pancreatic cancer, liver cancer, breast cancer, lung cancers (including, but not limited to, non-small cell lung carcinoma), renal cell carcinoma, prostate carcinoma, multiple myeloma, acute and chronic leukemias, T cell, B cell and NK cell lymphomas, brain tumors (including, but not limited to, neuroblastoma, glioblastoma, astrocytoma), squamous-cell carcinomas of the head and neck, and melanoma.

42. A method according to claim 23, wherein the disease, disorder, or condition is a cancer selected from the group consisting of chronic lymphocytic leukemia, acute lymphoblastic leukemia, chronic myelogenous leukemia, acute myeloid leukemia, adrenocortical carcinoma, anal cancer, appendix cancer, atypical teratoid/rhabdoid tumor, basal cell carcinoma, bile duct cancer, bladder cancer, bone cancer, brain tumor, brain and spinal cord tumor, brain stem glioma, central nervous system atypical teratoid/rhabdoid tumor, central nervous system embryonal tumors, breast cancer, bronchial tumors, Burkitt lymphoma, carcinoid tumor, carcinoma of unknown primary, central nervous system cancer, cervical cancer, childhood cancers, chordoma, chronic myeloproliferative disorders, colon cancer, craniopharyngioma, cutaneous T-cell lymphoma, ductal carcinoma in situ, embryonal tumors, endometrial cancer, ependymoblastoma, ependymoma, esophageal cancer, esthesioneuroblastoma, Ewing sarcoma, extracranial germ cell tumor, extrahepatic bile duct cancer, eye cancer, fibrous histiocytoma of bone, gallbladder cancer, gastric cancer, gastrointestinal carcinoid tumor, gastrointestinal stromal tumors, germ cell tumor, extracranial germ cell tumor, extragonadal germ cell tumor, ovarian germ cell tumor, gestational trophoblastic tumor, glioma, hairy cell leukemia, head and neck cancer, heart cancer, hepatocellular cancer, histiocytosis, Langerhans cell cancer, Hodgkin's lymphoma, hypopharyngeal cancer, intraocular melanoma, islet cell tumors, Kaposi sarcoma, kidney cancer, Langerhans cell histiocytosis, laryngeal cancer, lip and oral cavity cancer, lobular carcinoma in situ, lymphoma, AIDS-related lymphoma, macroglobulinemia, male breast cancer, medulloblastoma, medulloepithelioma, Merkel cell carcinoma, malignant mesothelioma, metastatic squamous neck cancer, midline tract carcinoma involving NUT gene, mouth cancer, multiple endocrine neoplasia syndrome, plasma cell neoplasm, mycosis fungoides, myeloma, chronic myeloproliferative disorder, nasal cavity cancer, paranasal sinus cancer, nasopharyngeal cancer, non-Hodgkin's lymphoma, oral cancer, oral cavity cancer, lip cancer, oropharyngeal cancer, osteosarcoma, ovarian cancer, papillomatosis, paraganglioma, paranasal sinus cancer, nasal cavity cancer, parathyroid cancer, penile cancer, pharyngeal cancer, pheochromocytoma, pineal parenchymal tumors of intermediate differentiation, pineoblastoma, pituitary tumor, plasma cell neoplasm, pleuropulmonary blastoma, primary central nervous system lymphoma, rectal cancer, renal cell cancer, renal pelvis cancer, ureter cancer, transitional cell cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, squamous neck cancer with occult primary, supratentorial primitive neuroectodermal tumors, testicular cancer, throat cancer, thymoma, thymic carcinoma, thyroid cancer, transitional cell cancer of the renal pelvis and ureter, gestational trophoblastic tumor, unknown primary, unusual cancer of childhood, urethral cancer, uterine cancer, uterine sarcoma, Waldenströms macroglobulinemia, or Wilms' tumor.

43. A method according to claim 23, wherein the compound is administered simultaneously or sequentially with a therapeutically effective amount of one or more other therapeutic agent(s) selected from the group consisting of anti-viral agents, chemotherapeutic agents (including alkylating antineoplastic agents, antimetabolites, anti-microtubule agents), immunosuppressants, anti-tumor vaccines, antiviral vaccines, cytokine therapy, tyrosine kinase inhibitors, immunotherapeutic agents, including checkpoint inhibitors such as PD-1, PD-L1 or CTLA-4 inhibitors and IDO/TDO inhibitors, adenosine A2A receptor antagonists, ectonucleotidase (CD73 and CD39) inhibitors, agent affecting interleukins, cytokines and chemokines, topoisomerase inhibitors, and cytotoxic antibiotics, or targeted therapies comprising antibodies, antibody drug conjugates, cell-based immunotherapy, nanoparticles, and radiotherapy.

44. A method according to claim 43, wherein the antibodies comprise a therapeutically effective amount of anti-PD-1, anti-PD-L1 or anti-CTLA4 antibodies.

45. A method for activating lymphocyte cells for releasing cytokines, comprising contacting the cell with at least one compound according to claim 1, or a pharmaceutically acceptable salt, solvate, or polymorph thereof, or with a pharmaceutical composition comprising a compound according to claim 1.

46. A method for protecting an organ during transport, comprising contacting the organ with a compound according to claim 1, or a pharmaceutically acceptable salt, solvate, or polymorph thereof.
